(19) United States
(12) Patent Application Publication
(10) Pub. No.: US 2025/0368666 A1
(43) Pub. Date: Dec. 4, 2025

(54) INDAZOLE COMPOUNDS

(71) Applicant: TYRA BIOSCIENCES, INC., Carlsbad, CA (US)

(72) Inventors: Robert L. HUDKINS, Carlsbad, CA (US); Daniel C. BENSEN, Carlsbad, CA (US)

(21) Appl. No.: 18/874,434

(22) PCT Filed: Jun. 29, 2023
(86) PCT No.: PCT/US2023/069358

§ 371 Date: Dec. 12, 2024

Related U.S. Application Data
(60) Provisional application No. 63/356,585, filed on Jun. 29, 2022.
(60) Provisional application No. 63/476,051, filed on Dec. 19, 2022.

(51) Int. Cl. C07F 9/6558; A61K 31/444; A61K 31/496; A61K 31/501; A61K 31/506; A61K 31/5377; A61K 31/5386; A61K 31/541; A61K 31/675; A61P 35/00; C07D 401/14; C07D 405/14; C07D 409/14; C07D 413/14; C07D 417/14; C07D 498/08; C07F 9/6584

(52) U.S. Cl. CPC C07F 9/65583; A61K 31/444; A61K 31/496; A61K 31/501; A61K 31/506; A61K 31/5377; A61K 31/5386; A61K 31/541; A61K 31/675; A61P 35/00; C07D 401/14; C07D 405/14; C07D 409/14; C07D 413/14; C07D 417/14; C07D 498/08; C07F 9/6584

(57) ABSTRACT

Disclosed herein are compounds and methods of treating diseases and/or conditions associated with FGFR inhibition.

### CROSS-REFERENCE TO RELATED APPLICATIONS

This application claims the benefit of U.S. Provisional Application No. 63/356,585, filed Jun. 29, 2022, and U.S. Provisional Application No. 63/476,051, filed Dec. 19, 2022. The entirety of each of these applications is incorporated by reference herein.

### TECHNICAL FIELD

The disclosure pertains to indazole compounds that are useful in treating cancer, pharmaceutical compositions that include one or more such indazole compounds, and methods of using such indazole compounds in treating cancer.

### BACKGROUND

Kinase inhibitors have been used to block the activity of kinases and thereby treat cancer (e.g., by inhibiting mitotic processes). These kinase inhibitors are often small molecules that target kinases to block the development, growth or spread of cancer.

However, although various inhibitors of kinases are known, there remains a need for selective inhibitors to be used for the treatment of diseases such as hyper-proliferative diseases, which offer one or more advantages over current compounds. Those advantages include: improved activity and/or efficacy; beneficial kinase selectivity profile according to the respective therapeutic need; improved side effect profile, such as fewer undesired side effects, lower intensity of side effects, or reduced (cyto)toxicity; improved targeting of mutant receptors in diseased cells; improved physicochemical properties, such as solubility/stability in water, body fluids, and/or pharmaceutical formulations; improved pharmacokinetic properties, allowing e.g. for dose reduction or an easier dosing scheme; easier drug substance manufacturing, e.g. by shorter synthetic routes or easier purification.

### SUMMARY

The compounds disclosed herein provide small molecule kinase inhibitors that are both efficacious and selective.

In some aspects, the disclosure is directed to compounds of formula (I):

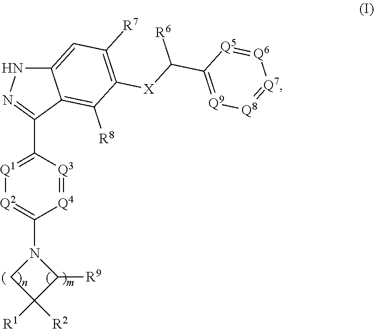

or a pharmaceutically acceptable salts thereof, wherein:
   X═O, S, or NR;
      R is H or C₁-C₃alkyl;
   n=1 or 2;
   m=1 or 2;
   R¹ is H, optionally substituted C₁-C₆alkyl, optionally substituted C₂-C₆alkenyl, C₃-C₆cycloalkyl, —C(O)NR³R⁴, —C(O)OR³, optionally substituted heterocycloalkyl, or optionally substituted heteroaryl;
   R⁹ is H or C₁-C₃alkyl;
   R² is H, optionally substituted C₁-C₆alkyl, —NR³R⁴, —OR^{4a}, —P(O)R^{4b}R^{4c}, —SO₂R³, or —C(O)NR³R⁴;
      R³ is H or C₁-C₆alkyl;
      R⁴ is H, optionally substituted C₁-C₆alkyl, optionally substituted C₂-C₆alkenyl, C₃-C₅cycloalkyl, 3- to 6-membered heterocycloalkyl, C(O)(CH₂)₂₋₃OH, or C(O)(CH₂)₀₋₃NR^{4d}R^{4e};
      or R³ and R⁴, together with the N atom to which they are both attached, form a 3- to 6-membered heterocycloalkyl optionally substituted with one or more substituents that are each C₁-C₆alkyl, C₁-C₆alkoxyl, F, or OH;
      or R³ and R⁴, together with the N atom to which they are both attached, form a 6- to 8-membered bridged heterocycloalkyl ring system;
      R^{4a} is H, optionally substituted C₁-C₆alkyl or C₃-C₅cycloalkyl;
      R^{4b} and R^{4c} are each independently C₁-C₆alkyl or —OC₁-C₆alkyl; or R^{4b} and R^{4c} together with the phosphorus atom to which they are both attached, form a 4- to 6-membered heterocycloalkyl ring;
      R^{4d} and R^{4e} are each independently H or C₁-C₆alkyl, or R^{4d} and R^{4e}, together with the nitrogen atom to which they are both attached, form a 4- to 6-membered heterocycloalkyl ring;
   or R¹ and R², together with the carbon atom to which they are both attached, form a 3-5 membered cycloalkyl ring;
   one or two of Q¹, Q², Q³, Q⁴ is N and the others are each independently CR^{5a};
      R^{5a} is H, halogen, —CN, —S(O)₂C₁-C₆alkyl, OCF₃, OC₁-C₃alkyl, or C₁-C₃alkyl; Q⁵, Q⁶, Q⁷, Q⁸, and Q⁹ are each independently N or CR⁵, wherein one or two of the Q⁵, Q⁶, Q⁷, Q⁸, and Q⁹ is N and the remainder are CR⁵;
      R⁵ is H, halogen, C₁-C₃alkyl, C₁-C₃alkoxyl, or cycloalkyl;
   R⁶ is C₁-C₆alkyl;
   R⁷ is H, halogen, —C₁-C₆alkyl, —C₁-C₆ alkoxyl, or -cycloalkyl; and
   R⁸ is H, halogen, —C₁-C₆alkyl, —C₁-C₆ alkoxyl, or -cycloalkyl.

In other aspects, the compounds of formula (I) are those wherein:
X═O, S, or NR;
R is H or C₁-C₃alkyl;
n=1 or 2;
m=1 or 2;
R¹ is H or optionally substituted C₁-C₆alkyl;
R² is H, optionally substituted C₁-C₆alkyl, —NR³R⁴, —OR^{4a}, —P(O)R^{4b}R^{4c}, —SO₂R³, or —C(O)NR³R⁴;
R⁹ is H.

R³ is H or C₁-C₆alkyl;
R⁴ is H, optionally substituted C₁-C₆alkyl, optionally substituted C₂-C₆alkenyl, C₃-C₅cycloalkyl, 3- to 6-membered heterocycloalkyl, or C(O)(CH₂)₀₋₃NR^{4d}R^{4e};
or R³ and R⁴, together with the N atom to which they are both attached, form a 3- to 6-membered heterocycloalkyl optionally substituted with one or more substituents that are each C₁-C₆alkyl, C₁-C₆alkoxyl, F, or OH;
R^{4a} is H, optionally substituted C₁-C₆alkyl or C₃-C₅cycloalkyl;
R^{4b} and R^{4c} are each independently C₁-C₆alkyl; or R^{4b} and R^{4c} together with the phosphorus atom to which they are both attached, form a 4- to 6-membered heterocycloalkyl ring;
   R^{4d} and R^{4e} are each independently H or C₁-C₆alkyl, or R^{4d} and R^{4e}, together with the nitrogen atom to which they are both attached, form a 4- to 6-membered heterocycloalkyl ring;
or R¹ and R², together with the carbon atom to which they are both attached, form a 3-5 membered cycloalkyl ring;
one or two of Q¹, Q², Q³, Q⁴ is N and the others are each independently CR^{5a};
   R^{5a} is H, halogen, —CN, —S(O)₂C₁-C₃alkyl, OCF₃, OC₁-C₃alkyl, or C₁-C₃alkyl;
Q⁵, Q⁶, Q⁷, Q⁸, and Q⁹ are each independently N or CR⁵, wherein one or two of the Q⁵, Q⁶, Q⁷, Q⁸, and Q⁹ is N and the remainder are CR⁵;
   R⁵ is H, halogen, C₁-C₃alkyl, C₁-C₃alkoxyl, or cycloalkyl;
R⁶ is C₁-C₆alkyl;
R⁷ is H, halogen, —C₁-C₆alkyl, —C₁-C₆alkoxyl, or -cycloalkyl; and
R⁸ is H, halogen, —C₁-C₆alkyl, —C₁-C₆alkoxyl, or -cycloalkyl.

Stereoisomers of the compounds of formula (I), and the pharmaceutical salts and solvates thereof, are also described. Methods of using compounds of formula (I) are described, as well as pharmaceutical compositions including the compounds of formula (I).

### DETAILED DESCRIPTION OF ILLUSTRATIVE EMBODIMENTS

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any subcombination.

The term “optionally substituted,” or “substituted” as used herein to describe a substituent defined herein, means that the substituent may, but is not required to be, substituted with one or more of: halo (i.e., —F, —C₁, —Br, —I), cyano, —OH, —C₁-C₆alkyl, C₃-C₆cycloalkyl, 3-7 membered heterocycloalkyl, —C₃-C₆spirocycloalkyl, 3-7 membered spiroheterocycloalkyl, bridged cycloalkyl, bridged heterocycloalkyl, C₂-C₆alkenyl, C₂-C₆ alkynyl, C₁-C₆haloalkyl (e.g., —CF₃; —CHF₂, —CH₂CF₃, and the like), —C₁-C₆alkoxy, —C₁-C₆ haloalkoxy (e.g., —OCF₃; —OCHF₂, —OCH₂CF₃, and the like), C₁-C₆alkylthio (e.g., —SCH₃; —SCH₂CH₃, and the like), C₁-C₆ alkylamino (e.g., —CH₂NH₂; —CH₂CH₂NH₂, and the like), —NH₂, —NH(C₁-C₆ alkyl), —N(C₁-C₆ alkyl)₂, —NH(C₁-C₆ alkoxy), —C(O)NHC₁-C₆alkyl, —C(O)N(C₁-C₆ alkyl)₂, —COOH, —C₁-C₆alkylCOOH, C₃-C₆cycloalkylCOOH, —C(O)NH₂, —C₁. C₆alkylCONH₂, —C₃-C₆cycloalkylCONH₂, —C₁-C₆alkylCONHC₁-C₆alkyl, —C₁-C₆alkylCON(C₁-C₆alkyl)₂, —C(O)C₁-C₆ alkyl, —C(O)OC₁-C₆ alkyl, —NHCO(C₁-C₆ alkyl), —N(C₁-C₆ alkyl)C(O)(C₁-C₆ alkyl), —S(O)C₁-C₆ alkyl, —S(O)₂C₁-C₆ alkyl, oxo (i.e., ═O), 6-12 membered aryl, or 5 to 12 membered heteroaryl groups. In other embodiments, “optionally substituted,” or “substituted” means that the substituent may, but is not required to be, substituted with one or more of —C(O)(C₁-C₆haloalkyl), —NHSO₂(C₁-C₆alkyl), —N(C₁-C₆alkyl)SO₂(C₁-C₆alkyl), or —P(O)(C₁-C₆alkyl)₂ (e.g., —P(O)(CH₃)₂). In other embodiments, “optionally substituted,” or “substituted” means that the substituent may, but is not required to be, substituted with one or more —P(O)(OC₁-6alkyl)₂ groups. In other embodiments, “optionally substituted,” or “substituted” means that the substituent may, but is not required to be, substituted with one or more of —C(O)NR′R″ wherein R′ and R″, together with the N atom to which they are both attached, form a 3-6 membered heterocycloalkyl ring. In other embodiments, “optionally substituted,” or “substituted” means that the substituent may, but is not required to be, substituted with one or more of —P(O)R′R″ wherein R′ and R″, together with the P atom to which they are both attached, form a 3-6 membered heterocycloalkyl ring. In some embodiments, each of the above optional substituents are themselves optionally substituted by one or two of these groups.

When a range of carbon atoms is used herein, for example, C₁-C₆, all ranges, as well as individual numbers of carbon atoms are encompassed. For example, “C₁-C₃” includes C₁-C₃, C₁-C₂, C₂-C₃, C₁, C₂, and C₃. Thus, for example, a “C₁ to C₄ alkyl” group refers to all alkyl groups having from 1 to 4 carbons (e.g., 1, 2, 3, or 4), that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. A “C₁ to C₆ alkyl” group refers to all alkyl groups having from 1 to 6 carbons (e.g., 1, 2, 3, 4, 5, or 6). A “C₀” means that the carbon atom is not present. Thus, for example, a “C₀-C₃ alkyl” group means that the alkyl group is absent, or that it is present and has 1, 2, or 3, carbon atoms.

As used herein, the term “alkyl” refers to a fully saturated aliphatic hydrocarbon group. The alkyl moiety may be branched or straight chain. Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 30 carbon atoms (whenever it appears herein, a numerical range such as “1 to 30” refers to each integer in the given range; e.g., “1 to 30 carbon atoms” means that the alkyl group may consist of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbon atoms, although the present definition also covers the occurrence of the term “alkyl” where no numerical range is designated). The “alkyl” group may also be a medium size alkyl having 1 to 12 carbon atoms. The “alkyl” group could also be a lower alkyl having 1 to 6 carbon atoms. An alkyl group may be substituted or unsubstituted, i.e., optionally substituted. By way of example only, “C₁-C₅ alkyl” indicates that there are one to five carbon atoms in the alkyl chain, i.e., the alkyl chain is selected from methyl, ethyl, n-propyl, isopropyl, n-butyl, isobutyl, tert-butyl, pentyl (branched and straight-chained), etc. Typical alkyl groups include, but are in no way limited to, methyl, ethyl, propyl, isopropyl, butyl, isobutyl, tertiary butyl, pentyl and hexyl. In several embodiments, “Me” is methyl (e.g., CH₃).

As used herein, “alkenyl” refers to an alkyl group that contains in the straight or branched hydrocarbon chain one or more double bonds. An alkenyl group may be unsubstituted or substituted, i.e., optionally substituted.

As used herein, “alkynyl” refers to an alkyl group that contains in the straight or branched hydrocarbon chain one or more triple bonds. An alkynyl group may be unsubstituted or substituted, i.e., optionally substituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups may contain between 3 and 12 carbon atoms. For example, a C₃-C₆cycloalkyl group indicates that there three to six carbon atoms in the ring, that is, the ring is a cyclopropyl, cyclobutyl, cyclopentyl, or cyclohexyl group. A cycloalkyl group may be unsubstituted or substituted, i.e., optionally substituted.

As used herein, the term “spirocycloalkyl ring” refers to a cycloalkyl ring that shares one carbon atom with another cyclic ring. For example, a 3-7 membered spirocycloalkyl ring indicates that there are 3, 4, 5, 6, or 7 carbon atoms in the cycloalkyl ring that shares a single carbon atom in common with another cyclic ring. By way of example, shown below are exemplary 3-7 membered spirocycloalkyl groups attached to a piperidine ring:

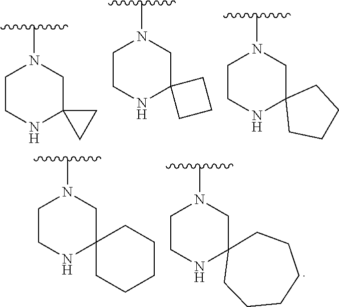

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be substituted or unsubstituted.

As used herein, “heteroaryl” refers to a monocyclic or multicyclic aromatic ring system (a ring system with fully delocalized pi-electron system) that contain(s) one or more heteroatoms, that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline, and triazine. Heteroaryl rings may also include bridge head nitrogen atoms. For example but not limited to: pyrazolo[1,5-a]pyridine, imidazo[1,2-a]pyridine, and pyrazolo[1,5-a]pyrimidine. A heteroaryl group may be substituted or unsubstituted.

As used herein, “heterocycloalkyl” refers to three-, four-, five-, six-, seven-, eight-, nine-, ten-, up to 18-membered monocyclic, bicyclic, and tricyclic ring system wherein carbon atoms together with from 1 to 5 heteroatoms constitute said ring system. A heterocycloalkyl may optionally contain one or more unsaturated bonds situated in such a way, however, that a fully delocalized pi-electron system does not occur throughout all the rings. The heteroatom(s) is an element other than carbon including, but not limited to, oxygen, sulfur, and nitrogen. A heterocycloalkyl may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused fashion. Additionally, any nitrogens in a heterocycloalkyl may be quaternized. Heterocycloalkyl groups may be unsubstituted or substituted. Examples of such “heterocycloalkyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone, and their benzo-fused analogs (e.g., benzimidazolidinone, tetrahydroquinoline, 3,4-methylenedioxyphenyl).

As used herein, the term “spiroheterocycloalkyl ring” refers to a heterocycloalkyl ring that shares one carbon atom with another cyclic ring. For example, a 3-7 membered spiroheterocycloalkyl ring indicates that there are 3, 4, 5, 6, or 7 atoms in the heterocycloalkyl ring, and only one of the carbon atoms in that heterocycloalkyl ring is also a member of another cyclic ring. By way of example, shown below are exemplary 3-7 membered spiroheterocycloalkyl groups attached to a piperidine ring:

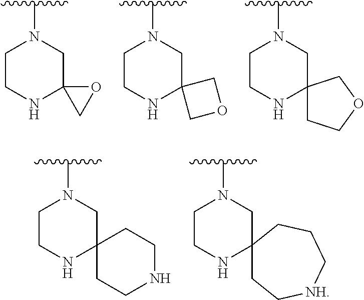

As used herein, the term “bridged bicyclic ring”, refers to a ring system comprising two joined cycloalkyl or heterocycloalkyl rings that share at least three at least three atoms. For example, a 6-9 membered bridged bicyclic ring indicates that there are 6, 7, 8, or 9 atoms in the bridged bicyclic ring. By way of example, shown below are exemplary 6-9 membered bridged bicyclic rings:

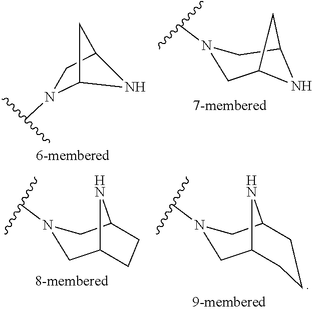 [SMILES: *N1CC2CC1N2 ; *N1CC2CC(C1)N2 ; *N1CC2CCC(C1)N2 ; *N1CC2CCCC(C1)N2]

As used herein, the term “amino” refers to a —NH₂ group.

As used herein, the term “hydroxy” refers to a —OH group.

As used herein, the term “halogen atom” or “halogen” refers to fluorine, chlorine, bromine and iodine.

The term “pharmaceutically acceptable salt” refers to a salt of a compound that does not cause significant irritation to an organism to which it is administered and does not abrogate the biological activity and properties of the compound. In several embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, salicylic, trifluoroacetic acid, or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition, it is understood that, in any compound described herein having one or more double bond(s) generating geometrical isomers that can be defined as E or Z, each double bond may independently be E or Z a mixture thereof. It is understood that, in any compound described herein having one or more chiral centers, all possible diastereomers are also envisioned. It is understood that, in any compound described herein all tautomers are envisioned. It is also understood that, in any compound described herein, all isotopes of the included atoms are envisioned. For example, any instance of hydrogen, may include hydrogen-1 (protium), hydrogen-2 (deuterium), hydrogen-3 (tritium) or other isotopes; any instance of carbon may include carbon-12, carbon-13, carbon-14, or other isotopes; any instance of oxygen may include oxygen-16, oxygen-17, oxygen-18, or other isotopes; any instance of fluorine may include one or more of fluorine-18, fluorine-19, or other isotopes; any instance of sulfur may include one or more of sulfur-32, sulfur-34, sulfur-35, sulfur-36, or other isotopes.

As used herein, the term “kinase inhibitor” means any compound, molecule or composition that inhibits or reduces the activity of a kinase. The inhibition can be achieved by, for example, blocking phosphorylation of the kinase (e.g., competing with adenosine triphosphate (ATP), a phosphorylating entity), by binding to a site outside the active site, affecting its activity by a conformational change, or by depriving kinases of access to the molecular chaperoning systems on which they depend for their cellular stability, leading to their ubiquitylation and degradation.

As used herein, “subject,” “host,” “patient,” and “individual” are used interchangeably and shall be given its ordinary meaning and shall also refer to an organism that has FGFR proteins. This includes mammals, e.g., a human, a non-human primate, ungulates, canines, felines, equines, mice, rats, and the like. The term “mammal” includes both human and non-human mammals.

The term “sample” or “biological sample” shall be given its ordinary meaning and also encompasses a variety of sample types obtained from an organism and can be used in an imaging, a diagnostic, a prognostic, or a monitoring assay. The term encompasses blood and other liquid samples of biological origin, solid tissue samples, such as a biopsy specimen or tissue cultures or cells derived therefrom and the progeny thereof.

The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

The terms “treatment,” “treating,” “treat” and the like shall be given its ordinary meaning and shall also include herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein shall be given its ordinary meaning and shall also cover any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, e.g., arresting its development; and/or (c) relieving the disease symptom, e.g., causing regression of the disease or symptom.

The terms “cancer,” “neoplasm,” and “tumor” are used interchangeably herein, shall be given its ordinary meaning and shall also refer to cells which exhibit relatively autonomous growth, so that they exhibit an aberrant growth phenotype characterized by a significant loss of control of cell proliferation. In general, cells of interest for detection or treatment in the present application include precursors, precancerous (e.g., benign), malignant, pre-metastatic, metastatic, and non-metastatic cells. As used herein, “FGFR related cancer” denotes those cancers that involve an increased activity in a mutant FGFR kinase, for example, the continued activation of FGFR.

The term “control” refers shall be given its ordinary meaning and shall also include a sample or standard used for comparison with a sample which is being examined, processed, characterized, analyzed, etc. In several embodiments, the control is a sample obtained from a healthy patient or a non-tumor tissue sample obtained from a patient diagnosed with a tumor. In several embodiments, the control is a historical control or standard reference value or range of values. In several embodiments, the control is a comparison to a wild-type FGFR arrangement or scenario.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

In some aspects, the disclosure is directed to compounds of formula (I):

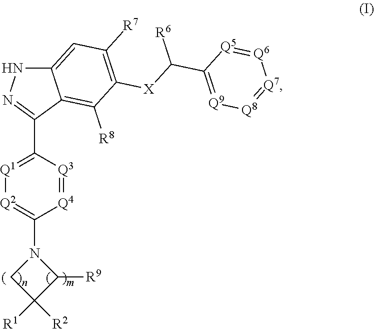

or a pharmaceutically acceptable salts thereof.

In some embodiments, R⁹ in the compounds of formula (I) is H.

In other embodiments, R⁹ in the compounds of formula (I) is C₁-C₃alkyl, such as, for example, C₃alkyl, C₂alkyl, C₁alkyl, methyl, ethyl, propyl, and the like.

In some embodiments, R⁹ in the compounds of formula (I) is methyl.

In some aspects, X in the compounds of formula (I) is O, S, or NR wherein R is H or C₁-C₃alkyl.

In some embodiments, X is O.

In other embodiments, X is S.

In some embodiments, X is NR.

In some embodiments, R is H.

In other embodiments, R is C₁-C₃alkyl, such as, for example, C₃alkyl, C₂alkyl, C₁alkyl, methyl, ethyl, propyl, and the like.

In some aspects, n in the compounds of formula (I) is 1 or 2.

In some embodiments, n is 1.

In some embodiments, n is 2.

In some aspects, m in the compounds of formula (I) is 1 or 2.

In some embodiments, m is 1.

In some embodiments, m is 2.

In some embodiments, n is 1 and m is 1.

In some embodiments, n is 1 and m is 2.

In some embodiments, n is 2 and m is 1.

In some embodiments, n is 2 and m is 2.

In some aspects, R¹ in the compounds of formula (I) is H, optionally substituted C₁-C₆alkyl, optionally substituted C₂-C₆alkenyl, C₃-C₅cycloalkyl, —C(O)NR³R⁴, —C(O)OR³, optionally substituted heterocycloalkyl, or optionally substituted heteroaryl.

In some aspects, R¹ in the compounds of formula (I) is H or optionally substituted C₁-C₆alkyl.

In some aspects, R¹ in the compounds of formula (I) is H or C₁-C₆alkyl.

In some aspects, R¹ in the compounds of formula (I) is H.

In some embodiments, R¹ is optionally substituted C₁-C₆alkyl, such as, for example, optionally substituted C₁-C₆alkyl, optionally substituted C₁-C₅alkyl, optionally substituted C₁-C₄alkyl, optionally substituted C₁-C₃alkyl, optionally substituted C₁-C₂alkyl, optionally substituted C₁alkyl, optionally substituted C₂alkyl, optionally substituted C₃alkyl, optionally substituted C₄alkyl, optionally substituted C₅alkyl, optionally substituted C₆alkyl, optionally substituted methyl, optionally substituted ethyl, optionally substituted n-propyl, optionally substituted isopropyl, optionally substituted n-butyl, optionally substituted isosbutyl, optionally substituted sec-butyl, optionally substituted pentanyl, optionally substituted hexanyl, and the like.

In some embodiments in which R¹ in the compounds of formula (I) is optionally substituted C₁-C₆alkyl, the optionally substituted C₁-C₆alkyl is —CH₂CH₂OH, —CH₂CH₂OCH₃, —CH₂CH₂F, —CH₂CHF₂, —CH₂CF₃, —CH₂CH₂CN, —CH₂CH₂CH₂OH, —CH₂OCH₃, —CH₂OCH(CH₃)₂, —CH₂OCH₂CH₃,

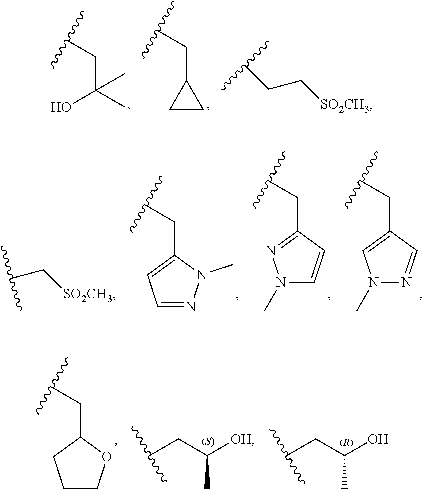

In some embodiments in which R¹ in the compounds of formula (I) is optionally substituted C₁-C₆alkyl, the optionally substituted C₁-C₆alkyl is

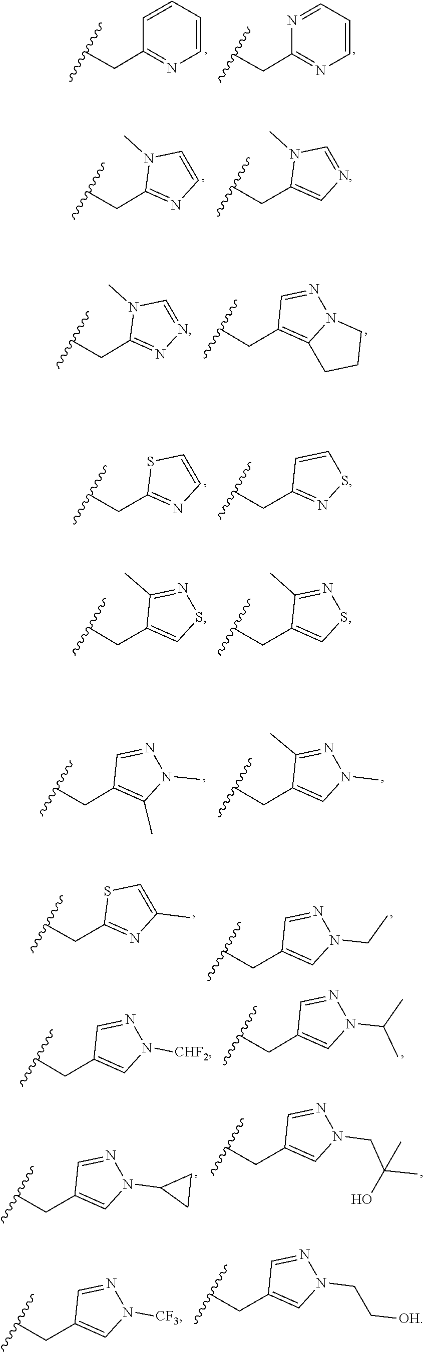

In some embodiments in which R¹ in the compounds of formula (I) is optionally substituted C₁-C₆alkyl, the optionally substituted C₁-C₆alkyl is

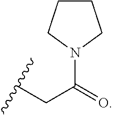 [SMILES: *CC(=O)N1CCCC1]

In some embodiments, R¹ is C₁-C₆alkyl, such as, for example, C₁-C₆alkyl, C₁-C₅alkyl, C₁-C₄alkyl, C₁-C₃alkyl, C₁-C₂alkyl, C₁alkyl, C₂alkyl, C₃alkyl, C₄alkyl, C₅alkyl, C₆alkyl, methyl, ethyl, n-propyl, isopropyl, n-butyl, isosbutyl, sec-butyl, pentanyl, hexanyl, and the like.

In some embodiments, R¹ in the compounds of formula (I) is methyl.

In other embodiments, R¹ in the compounds of formula (I) is isopropyl.

In other embodiments, R¹ in the compounds of formula (I) is isobutyl.

In other embodiments, R¹ in the compounds of formula (I) is sec-butyl.

In other embodiments, R¹ in the compounds of formula (I) is n-propyl.

In some embodiments, R¹ in the compounds of formula (I) is methyl substituted with cyano, i.e., —CH₂CN.

In some embodiments, R¹ in the compounds of formula (I) is methyl substituted with hydroxyl, i.e., —CH₂OH.

In other embodiments, R¹ in the compounds of formula (I) is ethyl.

In some aspects, R¹ in the compounds of formula (I) is optionally substituted C₂-C₆alkenyl, such as, for example, optionally substituted C₂-C₆alkenyl, optionally substituted C₂-C₅alkenyl, optionally substituted C₂-C₄alkenyl, optionally substituted C₂-C₃alkenyl, optionally substituted C₂alkenyl, optionally substituted C₃alkenyl, optionally substituted C₄alkenyl, optionally substituted C₅alkenyl, optionally substituted C₆alkenyl, optionally substituted ethenyl, optionally substituted n-propenyl, optionally substituted isopropenyl, optionally substituted n-butenyl, optionally substituted isosbutenyl, optionally substituted sec-butenyl, optionally substituted pentenyl, optionally substituted hexenyl, and the like.

In some embodiments, R¹ is unsubstituted C₂-C₆alkenyl.

In some embodiments, R¹ is substituted C₂-C₆alkenyl.

In some embodiments, R¹ is

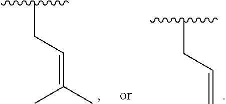

In some aspects, R¹ in the compounds of formula (I) is C₃-C₅cycloalkyl, such as, for example, C₃cycloalkyl, C₄cycloalkyl, C₅cycloalkyl, cyclopropyl, cyclobutyl, cyclopentyl, and the like.

In some aspects, R¹ in the compounds of formula (I) is cyclobutyl.

In some aspects, R¹ in the compounds of formula (I) is optionally substituted heterocycloalkyl.

In some embodiments, R¹ in the compounds of formula (I) is substituted heterocycloalkyl.

In other embodiments, R¹ in the compounds of formula (I) is unsubstituted heterocycloalkyl.

In some embodiments, R¹ in the compounds of formula (I) is

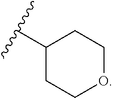 [SMILES: *C1CCOCC1]

In some aspects, R¹ in the compounds of formula (I) is optionally substituted heteroaryl.

In some embodiments, R¹ in the compounds of formula (I) is substituted heteroaryl.

In some embodiments, R¹ in the compounds of formula (I) is

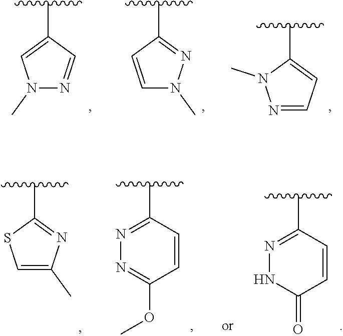

In some aspects, R¹ in the compounds of formula (I) is unsubstituted heteroaryl.

In some embodiments, R¹ in the compounds of formula (I) is

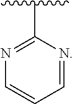 [SMILES: *c1ncccn1]

In some aspects, R¹ in the compounds of formula (I) is —C(O)OR³.

In some embodiments, R¹ in the compounds of formula (I) is

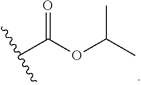 [SMILES: *C(=O)OC(C)C]

In some aspects, R¹ in the compounds of formula (I) is —C(O)NR³R⁴.

In some embodiments, R¹ in the compounds of formula (I) is

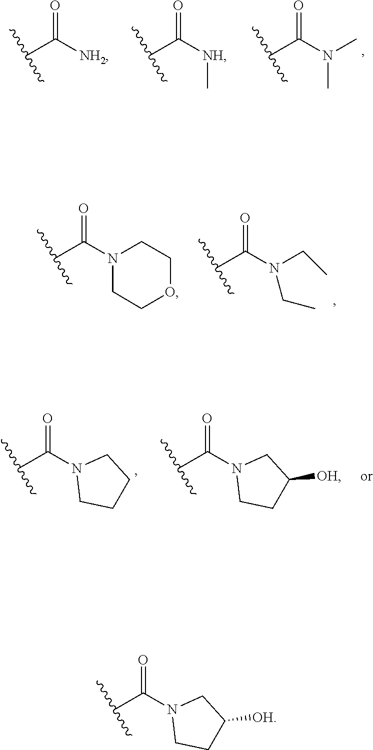

In some aspects, R² in the compounds of formula (I) is H, optionally substituted C₁-C₆alkyl, —NR³R⁴, —OR^{4a}, —P(O)R^{4b}R^{4c}, —SO₂R³, or —C(O)NR³R⁴.

In some aspects, R² in the compounds of formula (I) is H, optionally substituted C₁-C₆alkyl, —NR³R⁴, or —OR^{4a}.

In some embodiments, R² is H.

In some embodiments, R² is optionally substituted C₁-C₆alkyl, such as, for example, optionally substituted C₁C₆alkyl, optionally substituted C₁-C₅alkyl, optionally substituted C₁-C₄alkyl, optionally substituted C₁-C₃alkyl, optionally substituted C₁-C₂alkyl, optionally substituted C₁alkyl, optionally substituted C₂alkyl, optionally substituted C₃alkyl, optionally substituted C₄alkyl, optionally substituted C₅alkyl, optionally substituted C₆alkyl, optionally substituted methyl, optionally substituted ethyl, optionally substituted n-propyl, optionally substituted isopropyl, optionally substituted n-butyl, optionally substituted isosbutyl, optionally substituted sec-butyl, optionally substituted pentanyl, optionally substituted hexanyl, and the like.

In some embodiments, R² is unsubstituted C₁-C₆alkyl.

In some embodiments, R² is CH₃.

In some embodiments, R² is substituted C₁-C₆alkyl.

In some embodiments, R² is C₁-C₆alkyl substituted with —NHSO₂(C₁-C₆alkyl), —N(C₁-C₆alkyl)SO₂(C₁-C₆alkyl), 5- to 6-membered heterocycloakyl, —NH(C₁-C₆alkyl), —N(C₁-C₆alkyl)₂, or —P(O)(C₁-C₆alkyl)₂.

In some embodiments, R² is C₁-C₆alkyl substituted with —SO₂(C₁-C₆alkyl).

In some embodiments, R² is

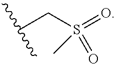 [SMILES: *CS(C)(=O)=O]

In some embodiments, R² is C₁-C₆alkyl substituted with —NH₂, such as, for example, —CH₂NH₂.

In some embodiments, R² is C₁-C₆alkyl substituted with —NHSO₂(C₁-C₆alkyl), such as, for example, NHSO₂(CH₃), NHSO₂(CH₂CH₃), NHSO₂(CH₂CH₂CH₃), NHSO₂(CH₂CH₂CH₂CH₃), and the like.

In some embodiments, R² is —CH₂—NHSO₂(CH₃).

In some embodiments, R² is C₁-C₆alkyl substituted with —N(C₁-C₆alkyl)SO₂(C₁-C₆alkyl), such as, for example, —N(CH₃)SO₂(CH₃), —N(CH₃)SO₂(CH₂CH₃), —N(CH₃)SO₂(CH₂ CH₂CH₃), —N(CH₂CH₃)SO₂(CH₃), —N(CH₂CH₃)SO₂(CH₂CH₃), and the like.

In some embodiments, R² is —CH₂—N(CH₃)SO₂(CH₃).

In some embodiments, R² is C₁-C₆alkyl substituted with 5- to 6-membered heterocycloakyl, such as, for example, pyrrolyl, furanyl, piperidinyl, piperazinyl, morpholinyl, and the like.

In some embodiments, R² is,

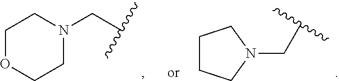 [SMILES: *CN1CCOCC1 ; *CN1CCCC1]

In some embodiments, R² is C₁-C₆alkyl substituted with —NH(C₁-C₆alkyl), such as, for example, —NH(C₆alkyl), —NH(C₅alkyl), —NH(C₄alkyl), —NH(C₃alkyl), —NH(C₂alkyl), —NH(C₁alkyl), NH(CH₃), NH(CH₂CH₃), and the like.

In some embodiments, R² is —CH₂—NH(CH₃) or —CH₂—NH(CH(CH₃)₂).

In some embodiments, R² is —CH₂—NH(CH₂CH₂OH).

In some embodiments, R² is C₁-C₆alkyl substituted with —N(C₁-C₆alkyl)₂, such as, for example, —N(C₆alkyl)₂, —N(C₅alkyl)₂, —N(C₄alkyl)₂, —N(C₃alkyl)₂, —N(C₂alkyl)₂, —N(C₁alkyl)₂, —N(CH₃)₂, —N(CH₂CH₃)₂, —N(CH₃)(CH₂CH₃), and the like.

In some embodiments, R² is —CH₂—N(CH₃)₂.

In some embodiments, R² is C₁-C₆alkyl substituted with —P(O)R^{4b}R^{4c}, wherein R^{4b} and R^{4c} are independently C₁-C₆alkyl or —OC₁-C₆alkyl; or R^{4b} and R^{4c} together with the phosphorus atom to which they are both attached, form a 4- to 6-membered heterocycloalkyl ring.

In some embodiments, R^{4b} is C₁-C₆alkyl, such as, for example, C₁-C₆alkyl, C₁-C₅alkyl, C₁-C₄alkyl, C₁-C₃alkyl, C₁-C₂alkyl, C₁alkyl, C₂alkyl, C₃alkyl, C₄alkyl, C₅alkyl, C₆alkyl, methyl, ethyl, n-propyl, isopropyl, n-butyl, isosbutyl, sec-butyl, pentanyl, hexanyl, and the like.

In some embodiments, R^{4b} is CH₃.

In some embodiments, R^{4b} is —OC₁-C₆alkyl, such as, for example, —OC₁-C₆alkyl, —OC₁-C₅alkyl, —OC₁-C₄alkyl, —OC₁-C₃alkyl, —OC₁-C₂alkyl, —OC₁alkyl, —OC₂alkyl, —OC₃alkyl, —OC₄alkyl, —OC₅alkyl, —OC₆alkyl, methoxyl, ethoxyl, n-propoxyl, isopropoxyl, n-butoxyl, isosbutoxyl, sec-butoxyl, and the like.

In some embodiments, R^{4b} is —OCH₃.

In some embodiments, R^{4b} is C₁-C₆alkyl, such as, for example, C₁-C₆alkyl, C₁-C₅alkyl, C₁-C₄alkyl, C₁-C₃alkyl, C₁-C₂alkyl, C₁alkyl, C₂alkyl, C₃alkyl, C₄alkyl, C₅alkyl, C₆alkyl, methyl, ethyl, n-propyl, isopropyl, n-butyl, isosbutyl, sec-butyl, pentanyl, hexanyl, and the like.

In some embodiments, R^{4b} is CH₃.

In some embodiments, R^{4b} is —OC₁-C₆alkyl, such as, for example, —OC₁-C₆alkyl, —OC₁-C₅alkyl, —OC₁-C₄alkyl, —OC₁-C₃alkyl, —OC₁-C₂alkyl, —OC₁alkyl, —OC₂alkyl, —OC₃alkyl, —OC₄alkyl, —OC₅alkyl, —OC₆alkyl, methoxyl, ethoxyl, n-propoxyl, isopropoxyl, n-butoxyl, isosbutoxyl, sec-butoxyl, and the like.

In some embodiments, R^{4c} is —OCH₃.

In some embodiments R² is C₁-C₆alkyl substituted with —P(O)(C₆alkyl)₂, —P(O)(C₅alkyl)₂, —P(O)(C₄alkyl)₂, —P(O)(C₃alkyl)₂, —P(O)(C₂alkyl)₂, —P(O)(C₁alkyl)₂, —P(O)(CH₃)₂, —P(O)(CH₂CH₃)₂, —P(O)(CH₃)(CH₂CH₃), and the like.

In some embodiments, R² is —CH₂—P(O)(CH₃)₂.

In some embodiments R² is C₁-C₆alkyl substituted with —P(O)(OC₆alkyl)₂, —P(O)(OC₅alkyl)₂, —P(O)(OC₄alkyl)₂, —P(O)(OC₃alkyl)₂, —P(O)(OC₂alkyl)₂, —P(O)(OC₁alkyl)₂, —P(O)(OCH₃)₂, —P(O)(OCH₂CH₃)₂, —P(O)(OCH₃)(OCH₂CH₃), and the like.

In some embodiments, R² is —CH₂—P(O)(OCH₃)₂.

In some embodiments, R^{4b} and R^{4c} together with the phosphorus atom to which they are both attached, form a 4- to 6-membered heterocycloalkyl ring, such as, for example, a 4-membered heterocycloalkyl, a 5-membered heterocycloalkyl, or a 6-membered heterocycloalkyl.

In some embodiments, R² is C₁-C₆alkyl substituted with:

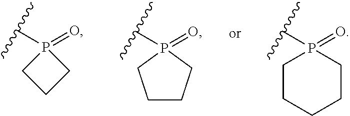 [SMILES: *P1(=O)CCC1 ; *P1(=O)CCCC1 ; *P1(=O)CCCCC1]

In some embodiments, R² is —NR³R⁴ wherein R³ is H or C₁-C₆alkyl; and R⁴ is H, optionally substituted C₁-C₆alkyl, optionally substituted C₂-C₆alkenyl, C₃-C₅cycloalkyl, 3- to 6-membered heterocycloalkyl, C(O)(CH₂)₂₋₃OH, or C(O)(CH₂)₀₋₃NR^{4d}R^{4e}; or R³ and R⁴, together with the N atom to which they are both attached, form a 3- to 6-membered heterocycloalkyl optionally substituted with one or more substituents that are each C₁-C₆alkyl, C₁-C₆alkoxyl, F, or OH.

In other embodiments, R² is —NR³R⁴ wherein R³ is H or C₁-C₆alkyl; and R⁴ is H, optionally substituted C₁-C₆alkyl, optionally substituted C₂-C₆alkenyl, C₃-C₅cycloalkyl, 3- to 6-membered heterocycloalkyl, or C(O)CH₂NR^{4d}R^{4e}; or R³ and R⁴, together with the N atom to which they are both attached, form a 3- to 6-membered heterocycloalkyl optionally substituted with one or more substituents that are each C₁-C₆alkyl, C₁-C₆alkoxyl, F, or OH.

In some embodiments, R² is —NR³R⁴ wherein R³ is H or C₁-C₆alkyl; and R⁴ is H, optionally substituted C₁-C₆alkyl, C₃-C₅cycloalkyl, 3- to 6-membered heterocycloalkyl, or C(O)CH₂NR^{4d}R^{4e}; or R³ and R⁴, together with the N atom to which they are both attached, form a 3- to 6-membered heterocycloalkyl optionally substituted with one or more substituents that are each C₁-C₆alkyl, C₁-C₆alkoxyl, F, or OH.

In some embodiments, R³ is H.

In some embodiments, R³ is C₁-C₆alkyl, such as, for example, C₁-C₆alkyl, C₁-C₅alkyl, C₁-C₄alkyl, C₁-C₃alkyl, C₁-C₂alkyl, C₁alkyl, C₂alkyl, C₃alkyl, C₄alkyl, C₅alkyl, C₆alkyl, methyl, ethyl, n-propyl, isopropyl, n-butyl, isosbutyl, sec-butyl, pentanyl, hexanyl, and the like.

In some embodiments, R³ is CH₃.

In some embodiments, R⁴ is H.

In some embodiments, R⁴ is optionally substituted C₁-C₆alkyl, such as, for example, optionally substituted C₁-C₆alkyl, optionally substituted C₁-C₅alkyl, optionally substituted C₁-C₄alkyl, optionally substituted C₁-C₃alkyl, optionally substituted C₁-C₂alkyl, optionally substituted C₁alkyl, optionally substituted C₂alkyl, optionally substituted C₃alkyl, optionally substituted C₄alkyl, optionally substituted C₅alkyl, optionally substituted C₆alkyl, optionally substituted methyl, optionally substituted ethyl, optionally substituted n-propyl, optionally substituted isopropyl, optionally substituted n-butyl, optionally substituted isosbutyl, optionally substituted sec-butyl, optionally substituted pentanyl, optionally substituted hexanyl, and the like.

In some embodiments, R⁴ is unsubstituted C₁-C₆alkyl.

In some embodiments, R⁴ is CH₃, —CH₂CH₃, or —CH(CH₃)₂.

In other embodiments, R⁴ is —CH₂CH(CH₃)₂, —CH₂CH₂CH₃, —CH(CH₃)CH₂CH₃,

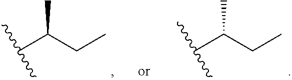

In some embodiments, R⁴ is substituted C₁-C₆alkyl.

In some embodiments, R⁴ is —CH₂-cyclopropyl, —CH₂CH₂SO₂CH₃, —CH₂CH₂CHF₂, —CH₂CHF₂, —CH₂CH₂OH, —CH₂CH₂OCH₃, —CH₂CH₂O-iso-Pr, —CH₂C(CH₃)₂OH, —CH₂CHCH₃OH, —CH₂CHOCH₃,

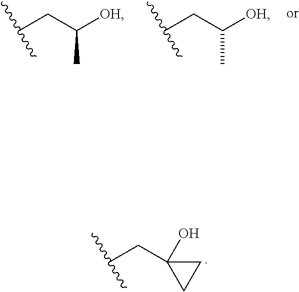

In some embodiments, R⁴ is —CH₂CH₂CN,

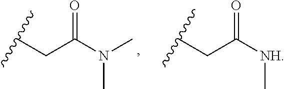

In some embodiments, R⁴ is —(CH₂)₁₋₃P(O)(C₁-C₆alkyl)₂, such as, for example, —CH₂P(O)(CH₃)₂, —CH₂CH₂P(O)(CH₂CH₃)₂, —CH₂CH₂P(O)(CH₂CH₂CH₃)₂, —CH₂CH₂P(O)(CH₃)(CH₂CH₃), and the like.

In some embodiments, R⁴ is —(CH₂)₁₋₃P(O), such as, for example, —CH₂P(O), —CH₂CH₂P(O), —CH₂CH₂P(O), —CH₂CH₂P(O), and the like.

In some embodiments, R⁴ is

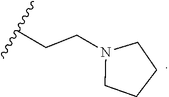 [SMILES: *CCN1CCCC1]

In some embodiments, R⁴ is optionally substituted C₂-C₆alkenyl, such as, for example, optionally substituted C₂-C₆alkenyl, optionally substituted C₂-C₅alkenyl, optionally substituted C₂-C₄alkenyl, optionally substituted C₂-C₃alkenyl, optionally substituted C₂alkenyl, optionally substituted C₃alkenyl, optionally substituted C₄alkenyl, optionally substituted C₅alkenyl, optionally substituted C₆alkenyl, optionally substituted ethenyl, optionally substituted n-propenyl, optionally substituted isopropenyl, optionally substituted n-butenyl, optionally substituted isosbutenyl, optionally substituted sec-butenyl, optionally substituted pentenyl, optionally substituted hexenyl, and the like.

In some embodiments, R⁴ is unsubstituted C₂-C₆alkenyl.

In some embodiments, R⁴ is —CH₂CH═CH₂.

In some embodiments, R⁴ is C₃-C₅cycloalkyl, such as, for example, cyclopropyl, cyclobutyl, or cyclopentyl.

In some embodiments, R⁴ is cyclobutyl.

In some embodiments, R⁴ is 3- to 6-membered heterocycloalkyl, such as, for example, 3-membered heterocycloalkyl, 4-membered heterocycloalkyl, 5-membered heterocycloalkyl, 6-membered heterocycloalkyl, aziridinyl, oxiranyl, azetidinyl, oxetanyl, pyrrolidinyl, tetrahydrofuranyl, tetrahydropyranyl, piperidinyl, piperazinyl, morpholinyl, and the like.

In some embodiments, R⁴ is oxetan-3-yl, thietane-3-yl-1,1-dioxide, tetrahydrofuran-3-yl,

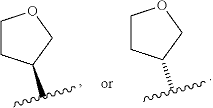 [SMILES: *[C@H]1CCOC1 ; *[C@@H]1CCOC1]

In some embodiments, R⁴ is C(O)(CH₂)₀₋₃NR^{4d}R^{4e}, such as, for example, C(O)NR^{4d}R^{4e}, C(O)(CH₂)NR^{4d}R^{4e}, C(O)(CH₂)₂NR^{4d}R^{4e}, or C(O)(CH₂)₃NR^{4d}R^{4e}.

In some embodiments, R⁴ is C(O)(CH₂)₀₋₃NR^{4d}R^{4e}, wherein R^{4d} and R^{4e} are each independently H or C₁-C₆alkyl, or R^{4d} and R^{4e}, together with the nitrogen atom to which they are both attached, form a 4- to 6-membered heterocycloalkyl ring.

In some embodiments, R⁴ is C(O)(CH₂)₀₋₃NR^{4d}R^{4e}, wherein R^{4d} and R^{4e} are each independently H or C₁-C₆alkyl.

In some embodiments, R⁴ is C(O)CH₂N(C₁-C₆alkyl)₂, such as, for example, C(O)CH₂N(C₆alkyl)₂, C(O)CH₂N(C₅alkyl)₂, C(O)CH₂N(C₄alkyl)₂, C(O)CH₂N(C₃alkyl)₂, C(O)CH₂N(C₂alkyl)₂, C(O)CH₂N(C₁alkyl)₂, C(O)CH₂N(CH₃)₂, C(O)CH₂N(CH₂CH₃)₂, C(O)CH₂N(CH₃)(CH₂CH₃), and the like.

In some embodiments, R⁴ is C(O)CH₂N(CH₃)₂.

In other embodiments, R⁴ is C(O)(CH₂)₀₋₃NR^{4d}R^{4e}, wherein R^{4d} and R^{4c}, together with the nitrogen atom to which they are both attached, form a 4- to 6-membered heterocycloalkyl ring, such as, for example, pyrrolidinyl, piperidinyl, morpholinyl, and the like.

In some embodiments, R³ and R⁴, together with the N atom to which they are both attached, form a 3- to 6-membered heterocycloalkyl optionally substituted with one or more substituents that are each C₁-C₆alkyl, C₁-C₆alkoxyl, F, or OH.

In some embodiments, R⁴ is C(O)(CH₂)₂₋₃OH, such as, for example, C(O)CH₂CH₂OH, or C(O)CH₂CH₂CH₂OH. In some embodiments, R⁴ is C(O)CH₂CH₂OH.

In some embodiments, R³ and R⁴, together with the N atom to which they are both attached, form an unsubstituted 3- to 6-membered heterocycloalkyl, such as, for example, 3-membered heterocycloalkyl, 4-membered heterocycloalkyl, 5-membered heterocycloalkyl, 6-membered heterocycloalkyl, aziridinyl, oxiranyl, azetidinyl, oxetanyl, pyrrolidinyl, tetrahydrofuranyl, tetrahydropyranyl, piperidinyl, piperazinyl, morpholinyl, and the like.

In some embodiments, R³ and R⁴, together with the N atom to which they are both attached, form an unsubstituted pyrrolidine-1-yl, azetidine-1-yl, or morpholin-4-yl.

In some embodiments, R³ and R⁴, together with the N atom to which they are both attached, form a substituted 3- to 6-membered heterocycloalkyl, such as, for example, substituted 3-membered heterocycloalkyl, substituted 4-membered heterocycloalkyl, substituted 5-membered heterocycloalkyl, substituted 6-membered heterocycloalkyl, substituted aziridinyl, substituted oxiranyl, substituted azetidinyl, substituted oxetanyl, substituted pyrrolidinyl, substituted tetrahydrofuranyl, substituted tetrahydropyranyl, substituted piperidinyl, substituted piperazinyl, substituted morpholinyl, and the like.

In some embodiments, R³ and R⁴, together with the N atom to which they are both attached, form a 3,3-dimethyl-azetidin-1-yl, 3-hydroxy-3-methylazetidin-1-yl, 1-methyl-4-azaphosphinan-4-yl 1-oxide, 1-methyl-4-azaphosphinan-4-yl 1-oxide, or 3-methoxy-3-methylazetidin-1-yl, 1-methyl-4-azaphosphinan-4-yl 1-oxide, or

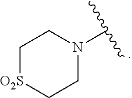 [SMILES: *N1CCS(=O)(=O)CC1]

In some embodiments, R³ and R⁴, together with the N atom to which they are both attached, form

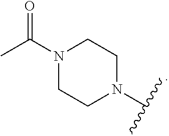 [SMILES: *N1CCN(C(C)=O)CC1]

In some embodiments, R³ and R⁴, together with the N atom to which they are both attached, form

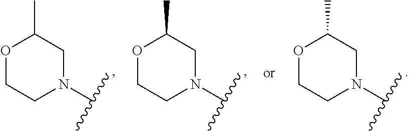

In some embodiments, R³ and R⁴, together with the N atom to which they are both attached, form a 6- to 8-membered bridged heterocycloalkyl ring system, such as, for example,

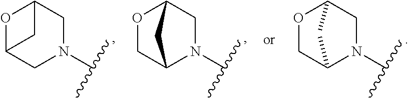

In some embodiments, R² is —OR^{4a} wherein R^{4a} is H, optionally substituted C₁-C₆alkyl or C₃-C₅cycloalkyl.

In some embodiments, R² is —OH.

In some embodiments, R^{4a} is H.

In some embodiments, R^{4a} is optionally substituted C₁-C₆alkyl, such as, for example, optionally substituted C₁-C₆alkyl, optionally substituted C₁-C₅alkyl, optionally substituted C₁-C₄alkyl, optionally substituted C₁-C₃alkyl, optionally substituted C₁-C₂alkyl, optionally substituted C₁alkyl, optionally substituted C₂alkyl, optionally substituted C₃alkyl, optionally substituted C₄alkyl, optionally substituted C₅alkyl, optionally substituted C₆alkyl, optionally substituted methyl, optionally substituted ethyl, optionally substituted n-propyl, optionally substituted isopropyl, optionally substituted n-butyl, optionally substituted isosbutyl, optionally substituted sec-butyl, optionally substituted pentanyl, optionally substituted hexanyl, and the like.

In some embodiments, R^{4a} is unsubstituted C₁-C₆alkyl.

In some embodiments, R^{4a} is —CH(CH₃).

In some embodiments, R^{4a} is substituted C₁-C₆alkyl.

In some embodiments, R^{4a} is —CH₂CH₂OH, or —CH₂C(CH₃)₂OH.

In some embodiments, R^{4a} is C₃-C₅cycloalkyl, such as, for example, cyclopropyl, cyclobutyl, or cyclopentyl.

In some embodiments, R^{4a} is cyclopropyl.

In some embodiments, R^{4a} is cyclobutyl.

In some embodiments, R^{4a} is cyclopentyl.

In some embodiments, R² is

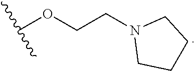 [SMILES: *OCCN1CCCC1]

In some embodiments, R² is —P(O)R^{4b}R^{4c}, wherein R^{4b} and R^{4c} are independently C₁-C₆alkyl; or R^{4b} and R^{4c} together with the phosphorus atom to which they are both attached, form a 4- to 6-membered heterocycloalkyl ring.

In some embodiments, R^{4b} is C₁-C₆alkyl, such as, for example, C₁-C₆alkyl, C₁-C₅alkyl, C₁-C₄alkyl, C₁-C₃alkyl, C₁-C₂alkyl, C₁alkyl, C₂alkyl, C₃alkyl, C₄alkyl, C₅alkyl, C₆alkyl, methyl, ethyl, n-propyl, isopropyl, n-butyl, isosbutyl, sec-butyl, pentanyl, hexanyl, and the like.

In some embodiments, R^{4b} is CH₃.

In some embodiments, R^{4c} is C₁-C₆alkyl, such as, for example, C₁-C₆alkyl, C₁-C₅alkyl, C₁-C₄alkyl, C₁-C₃alkyl, C₁-C₂alkyl, C₁alkyl, C₂alkyl, C₃alkyl, C₄alkyl, C₅alkyl, C₆alkyl, methyl, ethyl, n-propyl, isopropyl, n-butyl, isosbutyl, sec-butyl, pentanyl, hexanyl, and the like.

In some embodiments, R^{4c} is CH₃.

In some embodiments, R² is —P(O)(CH₃)₂.

In some embodiments, R^{4b} and R^{4c} together with the phosphorus atom to which they are both attached, form a 4- to 6-membered heterocycloalkyl ring, such as, for example, a 4-membered heterocycloalkyl, a 5-membered heterocycloalkyl, or a 6-membered heterocycloalkyl.

In some embodiments, R² is:

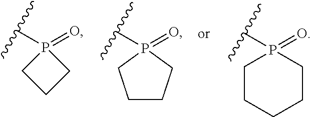

In some embodiments, R² is —SO₂R³, such as, for example, —SO₂H, or —SO₂C₁-C₆alkyl, such as, for example, —SO₂C₆alkyl, —SO₂C₅alkyl, —SO₂C₄alkyl, —SO₂C₃alkyl, —SO₂C₂alkyl, —SO₂C₁alkyl, or —SO₂CH₃.

In some embodiments, R² is —C(O)NR³R⁴, such as, for example, —C(O)NH₂, —C(O)NH(C₁-C₆alkyl), —C(O)N(C₁-C₆alkyl)(C₁-C₆alkyl), —C(O)NHCH₃, —C(O)N(CH₃)₂, —C(O)NHCH₂CH₃, —C(O)N(CH₂CH₃)₂, —C(O)NHCH₂CH₂OH, and the like.

In some aspects, R¹ and R² in the compounds of formula (I), together with the carbon atom to which they are both attached, form a 3-5 membered cycloalkyl ring, such as, for example, cyclopropyl, cyclobutyl, or cyclopentyl.

In some embodiments, R¹ and R² in the compounds of formula (I), together with the carbon atom to which they are both attached, form a cyclopropyl ring.

In some embodiments, R¹ and R² in the compounds of formula (I), together with the carbon atom to which they are both attached, form a cyclobutyl ring.

In some embodiments, R¹ and R² in the compounds of formula (I), together with the carbon atom to which they are both attached, form a cyclopentyl ring.

In some aspects, one or two of Q¹, Q², Q³, Q⁴ in the compounds of formula (I) are N and the others are each independently CR^{5a}.

In some embodiments, Q¹ is N and Q², Q³, and Q⁴ are each independently CR^{5a}.

In some embodiments, Q² is N and Q¹, Q³, and Q⁴ are each independently CR^{5a}.

In some embodiments, Q³ is N and Q¹, Q², and Q⁴ are each independently CR^{5a}.

In some embodiments, Q⁴ is N and Q¹, Q², and Q³ are each independently CR^{5a}.

In other embodiments, two of Q¹, Q², Q³, Q⁴ is N and the others are each independently CR^{5a}.

In some embodiments, Q¹ and Q² are each N, and Q³, and Q⁴ are each independently CR^{5a}.

In some embodiments, Q¹ and Q³ are each N, and Q² and Q⁴ are each independently CR^{5a}.

In some embodiments, Q¹ and Q⁴ are each N, and Q² and Q³ are each independently CR^{5a}.

In some embodiments, Q² and Q³ are each N, and Q¹ and Q⁴ are each independently CR^{5a}.

In some embodiments, Q² and Q⁴ are each N, and Q¹ and Q³ are each independently CR^{5a}.

In some embodiments, Q³ and Q⁴ are each N, and Q¹ and Q² are each independently CR^{5a}.

In some aspects of the compounds of formula (I), each R^{5a} is independently H, halogen, —CN, —S(O)₂C₁-C₃alkyl, OCF₃, OC₁-C₃alkyl, or C₁-C₃alkyl.

In some embodiments, at least one R^{5a} is H.

In some embodiments, at least one R^{5a} is halogen, i.e., —F, —C₁, —Br, or —I.

In some embodiments, at least one R^{5a} is —F.

In some embodiments, at least one R^{5a} is —CN.

In some embodiments at least one R^{5a} is —SO₂C₁-C₃alkyl, such as, for example, —SO₂C₁alkyl, —SO₂C₂alkyl, —SO₂C₃alkyl, —SO₂CH₂CH₃, —SO₂CH₃, and the like. In some embodiments, at least one R^{5a} is —SO₂CH₃.

In some embodiments, R^{5a} is OCF₃.

In some embodiments, at least one R^{5a} is OC₁-C₃alkyl, such as, for example, OC₁-C₃alkyl, OC₁-C₂alkyl, OC₁alkyl, OC₂alkyl, OC₃alkyl, —OCH₃, —OCH₂CH₃, —Opropyl, and the like. In some embodiments at least one R^{5a} is —OCH₃.

In some embodiments, at least one R^{5a} is C₁-C₃alkyl, such as, for example, C₁-C₃alkyl, C₁-C₂alkyl, C₁alkyl, C₂alkyl, C₃alkyl, —CH₃, —CH₂CH₃, -propyl, and the like. In some embodiments at least one R^{5a} is —CH₃.

In some aspects, Q⁵, Q⁶, Q⁷, Q⁸, and Q⁹ in the compounds of formula (I) are each independently N or CR⁵, wherein one or two of Q⁵, Q⁶, Q⁷, Q⁸, and Q⁹ is N and the remainder are each independently CR⁵.

In some embodiments, one of Q⁵, Q⁶, Q⁷, Q⁸, or Q⁹ is N, and the remainder are each independently CR⁵.

In some embodiments, Q⁵ is N and Q⁶, Q⁷, Q⁸, and Q⁹ are each independently CR⁵.

In some embodiments, Q⁶ is N and Q⁵, Q⁷, Q⁸, and Q⁹ are each independently CR⁵.

In some embodiments, Q⁷ is N and Q⁵, Q⁶, Q⁸, and Q⁹ are each independently CR⁵.

In some embodiments, Q⁸ is N and Q⁵, Q⁶, Q⁷, and Q⁹ are each independently CR⁵.

In some embodiments, Q⁹ is N and Q⁵, Q⁶, Q⁷, and Q⁸ are each independently CR⁵.

In other embodiments, two of Q⁵, Q⁶, Q⁷, Q⁸, or Q⁹ is each N, and the remainder are each independently CR⁵.

In some embodiments, Q⁵ and Q⁶ are each N, and Q⁷, Q⁸, and Q⁹ are each independently CR⁵.

In some embodiments, Q⁵ and Q⁷ are each N, and Q⁶, Q⁸, and Q⁹ are each independently CR⁵.

In some embodiments, Q⁵ and Q⁸ are each N, and Q⁶, Q⁷, and Q⁹ are each independently CR⁵.

In some embodiments, Q⁵ and Q⁹ are each N, and Q⁶, Q⁷, and Q⁸ are each independently CR⁵.

In some embodiments, Q⁶ and Q⁷ are each N, and Q⁵, Q⁷, and Q⁹ are each independently CR⁵.

In some embodiments, Q⁶ and Q⁸ are each N, and Q⁵, Q⁷, and Q⁹ are each independently CR⁵.

In some embodiments, Q⁶ and Q⁹ are each N, and Q⁵, Q⁷, and Q⁹ are each independently CR⁵.

In some embodiments, Q⁷ and Q⁸ are each N, and Q⁵, Q⁶, and Q⁹ are each independently CR⁵.

In some embodiments, Q⁷ and Q⁹ are each N, and Q⁵, Q⁶, and Q⁹ are each independently CR⁵.

In some embodiments, Q⁸ and Q⁹ are each N, and Q⁵, Q⁶, and Q⁷ are each independently CR⁵.

In some aspects of the disclosure, each R⁵ in the compounds of formula (I), is independently H, halogen, C₁-C₃alkyl, C₁-C₃alkoxyl, or cycloalkyl.

In some embodiments of the compounds of formula (I), at least one R⁵ is H.

In some embodiments of the compounds of formula (I), at least one R⁵ is halogen, such as, —F, —C₁, —Br, or —I.

In some embodiments, at least one R⁵ is —C₁.

In some embodiments of the compounds of formula (I), at least one R⁵ is C₁-C₃alkyl, such as, for example, C₁-C₃alkyl, C₁-C₂alkyl, C₁alkyl, C₂alkyl, C₃alkyl, —CH₃, —CH₂CH₃, -propyl, and the like.

In some embodiments, at least one R⁵ is —CH₃.

In some embodiments of the compounds of formula (I), at least one R⁵ is C₁-C₃alkoxyl, such as, for example, C₁-C₃alkoxyl, C₁-C₂alkoxyl, C₁alkoxyl, C₂alkoxyl, C₃alkoxyl, —OCH₃, —OCH₂CH₃, -propoxyl, and the like.

In some embodiments, at least one R⁵ is —OCH₃.

In some embodiments of the compounds of formula (I), at least one R⁵ is cycloalkyl, such as, for example, cyclopropyl, cyclobutyl, cyclopentyl, cyclohexyl, or cycloheptyl, and the like.

In some embodiments of the compounds of formula (I), two R⁵ are halogen, and the remaining R⁵ are H.

In other embodiments of the compounds of formula (I), two R⁵ are —C₁, and the remaining R⁵ are H.

In some embodiments of the compounds of formula (I), Q⁵ and Q⁹ are each independently CR⁵ wherein each R⁵ is independently a halogen; Q⁶ and Q⁸ are each independently CR⁵ wherein each R⁵ is H; and Q⁷ is N.

In some embodiments of the compounds of formula (I), Q⁵, Q⁸, and Q⁹ are each independently CR⁵ wherein each R⁵ is independently a halogen; Q⁶ is CR⁵ wherein R⁵ is H; and Q⁷ is N.

In some embodiments of the compounds of formula (I), Q⁵ and Q⁹ are each independently CR⁵ wherein each R⁵ is —Cl; Q⁶ and Q⁸ are each independently CR⁵ wherein R⁵ is H; and Q⁷ is N.

In some embodiments of the compounds of formula (I), Q⁵ and Q⁹ are each independently CR⁵ wherein each R⁵ is —Cl; Q⁶ is CR⁵ wherein R⁵ is H; Q⁸ is CR⁵ wherein R⁵ is —F; and Q⁷ is N.

In some embodiments of the compounds of formula (I), Q⁵ and Q⁹ are each independently CR⁵ wherein each R⁵ is independently halogen; Q⁶ is CR⁵ wherein R⁵ is H and Q⁸ is CR⁵ wherein R⁵ is C₁-C₃alkyl; and Q⁷ is N.

In some embodiments of the compounds of formula (I), Q⁵ and Q⁹ are each independently CR⁵ wherein each R⁵ is —Cl; Q⁶ is CR⁵ wherein R⁵ is H and Q⁸ is CR⁵ wherein R⁵ is —CH₃; and Q⁷ is N.

In some embodiments of the compounds of formula (I), Q⁵ and Q⁹ are each independently CR⁵ wherein each R⁵ is halogen; Q⁶ is CR⁵ wherein R⁵ is H and Q⁸ is N; and Q⁷ is CR⁵ wherein R⁵ is H.

In some embodiments of the compounds of formula (I), Q⁵ and Q⁹ are each independently CR⁵ wherein each R⁵ is —Cl; Q⁶ is CR⁵ wherein R⁵ is H and Q⁸ is N; and Q⁷ is CR⁵ wherein R⁵ is H.

In some embodiments of the compounds of formula (I), Q⁵ and Q⁹ are each independently CR⁵ wherein each R⁵ is independently a halogen; Q⁶ is CR⁵ wherein R⁵ is H and Q⁸ is N; and Q⁷ is N.

In some embodiments of the compounds of formula (I), Q⁵ and Q⁹ are each independently CR⁵ wherein each R⁵ is —Cl; Q⁶ is CR⁵ wherein R⁵ is H and Q⁸ is N; and Q⁷ is N.

In some embodiments of the compounds of formula (I), Q⁵ and Q⁹ are each independently CR⁵ wherein each R⁵ is independently a C₁-C₃alkyl; Q⁶ is CR⁵ wherein R⁵ is H and Q⁸ is N; and Q⁷ is N.

In some embodiments of the compounds of formula (I), Q⁵ and Q⁹ are each independently CR⁵ wherein each R⁵ is —CH₃; Q⁶ is CR⁵ wherein R⁵ is H and Q⁸ is N; and Q⁷ is N.

In some aspects of the disclosure, R⁶ in the compounds of formula (I) is C₁-C₆alkyl, such as, for example, C₁-C₆alkyl, C₁-C₅alkyl, C₁-C₄alkyl, C₁-C₃alkyl, C₁-C₂alkyl, C₁alkyl, C₂alkyl, C₃alkyl, C₄alkyl, C₅alkyl, C₆alkyl, methyl, ethyl, n-propyl, isopropyl, n-butyl, isosbutyl, sec-butyl, pentanyl, hexanyl, and the like.

In some embodiments of the compounds of the disclosure, R⁶ is —CH₃.

In some aspects, R⁷ in the compounds of formula (I) is H, halogen, —C₁-C₆alkyl, —C₁-C₆alkoxyl, or -cycloalkyl.

In some embodiments, R⁷ in the compounds of formula (I) is H.

In some embodiments, R⁷ in the compounds of formula (I) is halogen, such as, for example, —F, —C₁, —Br, or —I.

In some embodiments, R⁷ in the compounds of formula (I) is —F.

In other embodiments, R⁷ in the compounds of formula (I) is —C₁.

In some embodiments, R⁷ in the compounds of formula (I) is —C₁-C₆alkyl, such as, for example, substituted or unsubstituted: C₁-C₆alkyl, C₁-C₅alkyl, C₁-C₄alkyl, C₁-C₃alkyl, C₁-C₂alkyl, C₁alkyl, C₂alkyl, C₃alkyl, C₄alkyl, C₅alkyl, C₆alkyl, methyl, ethyl, n-propyl, isopropyl, n-butyl, isosbutyl, sec-butyl, pentanyl, hexanyl, and the like.

In some embodiments, R⁷ is —CH₃.

In some embodiments, R⁷ in the compounds of formula (I) is —C₁-C₆ alkoxyl, such as, for example, —C₁-C₆alkoxyl, —C₁-C₅alkoxyl, —C₁-C₄alkoxyl, —C₁-C₃alkoxyl, —C₁-C₂alkoxyl, —C₁alkoxyl, —C₂alkoxyl, —C₃alkoxyl, —C₄alkoxyl, —C₅alkoxyl, —C₆alkoxyl, —OCH₃, —OCH₂CH₃, -propoxyl, and the like.

In some embodiments, R⁷ is —OCH₃.

In some embodiments, R⁷ in the compounds of formula (I) is -cycloalkyl, such as, for example, cyclopropyl, cyclobutyl, cyclopentyl, cyclohexyl, or cycloheptyl, and the like.

In some aspects, R⁸ is H, halogen, —C₁-C₆alkyl, —C₁-C₆alkoxyl, or -cycloalkyl.

In some embodiments, R⁸ in the compounds of formula (I) is H.

In some embodiments, R⁸ in the compounds of formula (I) is halogen, such as, for example, —F, —C₁, —Br, or —I.

In some embodiments, R⁸ in the compounds of formula (I) is —F.

In some embodiments, R⁸ in the compounds of formula (I) is —C₁-C₆alkyl, such as, for example, substituted or unsubstituted: C₁-C₆alkyl, C₁-C₅alkyl, C₁-C₄alkyl, C₁-C₃alkyl, C₁-C₂alkyl, C₁alkyl, C₂alkyl, C₃alkyl, C₄alkyl, C₅alkyl, C₆alkyl, methyl, ethyl, n-propyl, isopropyl, n-butyl, isosbutyl, sec-butyl, pentanyl, hexanyl, and the like.

In some embodiments, R⁸ is —CH₃.

In some embodiments, R⁸ in the compounds of formula (I) is —C₁-C₆ alkoxyl, such as, for example, —C₁-C₆alkoxyl, —C₁-C₅alkoxyl, —C₁-C₄alkoxyl, —C₁-C₃alkoxyl, —C₁-C₂alkoxyl, —C₁alkoxyl, —C₂alkoxyl, —C₃alkoxyl, —C₄alkoxyl, —C₅alkoxyl, —C₆alkoxyl, —OCH₃, —OCH₂CH₃, -propoxyl, and the like.

In some embodiments, R⁸ is —OCH₃.

In some embodiments, R⁸ in the compounds of formula (I) is -cycloalkyl, such as, for example, cyclopropyl, cyclobutyl, cyclopentyl, cyclohexyl, or cycloheptyl, and the like.

In some embodiments of the compounds of formula (I), R⁷ is —F and R⁸ is H.

In some embodiments of the compounds of formula (I), R⁷ is —C₁ and R⁸ is H.

In some embodiments of the compounds of formula (I), R⁷ is —CH₃ and R⁸ is H.

In some embodiments of the compounds of formula (I), R⁷ is —OCH₃ and R⁸ is H.

In some embodiments of the compounds of formula (I), R⁷ is —H and R⁸ is —F.

In some embodiments of the compounds of formula (I), R⁷ is —H and R⁸ is —C₁.

In some embodiments of the compounds of formula (I), R⁷ is —H and R⁸ is —CH₃.

In some embodiments of the compounds of formula (I), R⁷ is —H and R⁸ is OCH₃.

In some aspects, the disclosure is directed to the compounds of formula (I) that are compounds of formula (IA):

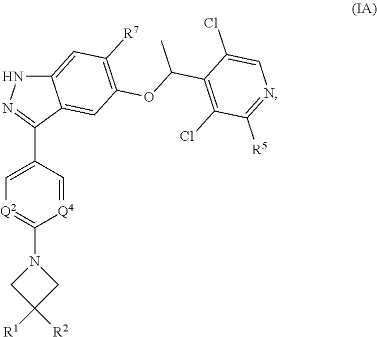

or a pharmaceutically acceptable salt thereof, wherein
   Q² and Q⁴ are each N, or one of Q² or Q⁴ is N and the other is CR^{5a}.
      R^{5a} is H, F, —SO₂CH₃, or —CN;
   R⁵ is H or CH₃; and
   R⁷ is H, F, or OCH₃,
and R¹ and R² are as described above for formula (I).

In some embodiments of the compounds of formula (IA), R⁵ is H.

In some embodiments of the compounds of formula (IA), R⁵ is CH₃.

In some embodiments of the compounds of formula (IA), R⁷ is H.

In some embodiments of the compounds of formula (IA), R⁷ is F.

In some embodiments of the compounds of formula (IA), R⁷ is OCH₃.

In some embodiments of the compounds of formula (IA), Q² and Q⁴ are each N.

In some embodiments of the compounds of formula (IA), one of Q² or Q⁴ is N and the other is CR^{5a}.

In some embodiments of the compounds of formula (IA) wherein one of Q² or Q⁴ is N and the other is CR^{5a}, R^{5a} is H or F.

In some embodiments of the compounds of formula (IA) one of Q² or Q⁴ is N and the other is CR^{5a}, R^{5a} is H.

In some embodiments of the compounds of formula (IA) one of Q² or Q⁴ is N and the other is CR^{5a}, R^{5a} is F.

In some embodiments of the compounds of formula (IA) one of Q² or Q⁴ is N and the other is CR^{5a}, R^{5a} is CN or SO₂CH₃.

In some embodiments of the compounds of formula (IA) one of Q² or Q⁴ is N and the other is CR^{5a}, R^{5a} is CN.

In some embodiments of the compounds of formula (IA) one of Q² or Q⁴ is N and the other is CR^{5a}, R^{5a} is SO₂CH₃.

In some embodiments, the compounds of formula (IA) are compounds of formula (IA-1-1):

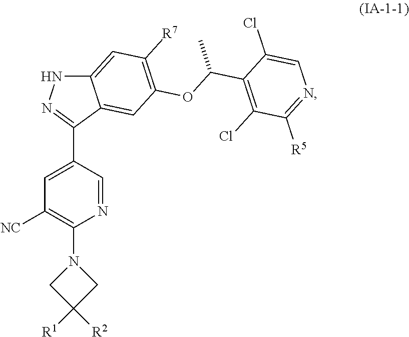

or a pharmaceutically acceptable salt thereof, wherein
   R⁵ is H or CH₃; and
   R⁷ is H, F, or OCH₃; and
   R¹ and R² are as described above for formula (I).

In some embodiments of the compounds of formula (IA-1-1), R⁵ is H.

In some embodiments of the compounds of formula (IA-1-1), R⁵ is CH₃.

In some embodiments of the compounds of formula (IA-1-1), R⁷ is H.

In some embodiments of the compounds of formula (IA-1-1), R⁷ is F.

In some embodiments of the compounds of formula (IA-1-1), R⁷ is OCH₃.

In some embodiments of the compounds of formula (IA-1-1), R¹ is optionally substituted C₁-C₆alkyl, such as, for example, methyl, ethyl, n-propyl, isopropyl, isosbutyl, sec-butyl, —CH₂CH₂OH, —CH₂CH₂OCH₃, —CH₂CH₂F, —CH₂CHF₂, —CH₂CH₂CN, —CH₂CH₂CH₂OH, —CH₂OCH₃, —CH₂OCH(CH₃)₂, —CH₂OCH₂CH₃,

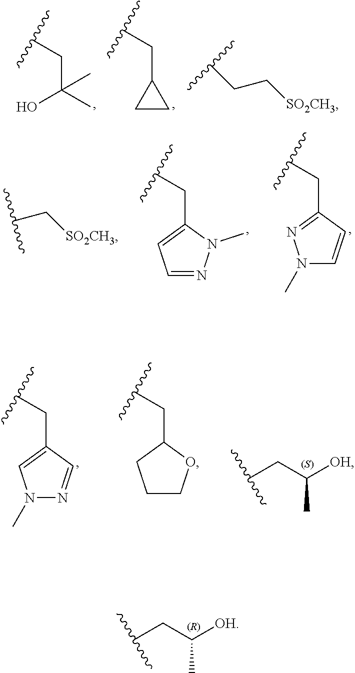

In some embodiments of the compounds of formula (IA-1-1), R¹ is optionally substituted C₂-C₆alkenyl, such as, for example,

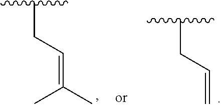

In some embodiments of the compounds of formula (IA-1-1), R¹ is C₃-C₆cycloalkyl, such as, for example, cyclobutyl.

In some embodiments of the compounds of formula (IA-1-1), R¹ is H.

In some embodiments of the compounds of formula (IA-1-1), R² is optionally substituted C₁-C₆alkyl, —NR³R⁴, or —OR^{4a}.

In some embodiments, the compounds of formula (IA) are compounds of formula (IA-1):

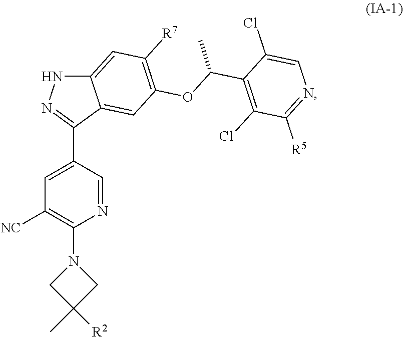

or a pharmaceutically acceptable salt thereof, wherein
   R⁵ is H or CH₃; and
   R⁷ is H, F, or OCH₃; and
   R² is as described above for formula (I).

In some embodiments of the compounds of formula (IA-1), R⁵ is H.

In some embodiments of the compounds of formula (IA-1), R⁵ is CH₃.

In some embodiments of the compounds of formula (IA-1), R⁷ is H.

In some embodiments of the compounds of formula (IA-1), R⁷ is F.

In some embodiments of the compounds of formula (IA-1), R⁷ is OCH₃.

In some embodiments of the compounds of formula (IA-1), R² is optionally substituted C₁-C₆alkyl.

In some embodiments, of the compounds of formula (IA-1), R² is CH₃, —CH₂—NHSO₂(CH₃), —CH₂—N(CH₃)SO₂(CH₃),

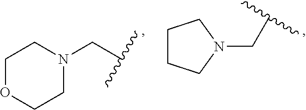

—CH₂—NH(CH₃), —CH₂—NH(—CH(CH₃)₂), —CH₂—N(CH₃)₂, or —CH₂—P(O)(CH₃)₂.

In some embodiments of the compounds of formula (IA-1), R² is —NR³R⁴ wherein R³ and R⁴ are as defined above for formula (I).

In some embodiments of compounds of formula (IA-1), R³ is H.

In some embodiments of compounds of formula (IA-1), R³ is CH₃.

In some embodiments of compounds of formula (IA-1), R⁴ is H.

In some embodiments of compounds of formula (IA-1), R⁴ is CH₃, —CH₂CH₃, —CH(CH₃), —CH₂-cyclopropyl, —CH₂CH₂CHF₂, —CH₂CHF₂, —CH₂CH₂OH, —CH₂C(CH₃)₂OH, —CH₂CHOCH₃, —C(O)CH₂N(CH₃)₂,

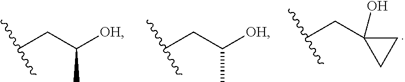

In other embodiments of compounds of formula (IA-1), R⁴ is —CH₂CH(CH₃)₂, —CH₂CH₂CH₃, —CH(CH₃)CH₂CH₃,

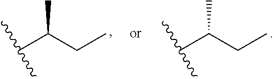

In some embodiments, R⁴ is —CH₂CH═CH₂.

In some embodiments of compounds of formula (IA-1), R⁴ is cyclopropyl, cyclobutyl, or cyclopentyl.

In some embodiments of compounds of formula (IA-1), R⁴ is oxetan-3-yl, thietane-3-yl-1,1-dioxide, tetrahydrofuran-3-yl,

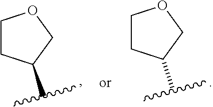 [SMILES: *[C@H]1CCOC1 ; *[C@@H]1CCOC1]

In some embodiments of compounds of formula (IA-1), R⁴ is C(O)CH₂N(CH₃)₂.

In some embodiments of compounds of formula (IA-1), R³ and R⁴, together with the N atom to which they are both attached, form a 3- to 6-membered heterocycloalkyl optionally substituted with one or more substituents that are each C₁-C₆alkyl, C₁-C₆alkoxyl, or OH.

In some embodiments, R³ and R⁴, together with the N atom to which they are both attached, form a 3,3-dimethyl-azetidin-1-yl, 3-hydroxy-3-methylazetidin-1-yl, 1-methyl-4-azaphosphinan-4-yl 1-oxide, 1-methyl-4-azaphosphinan-4-yl 1-oxide, or 3-methoxy-3-methylazetidin-1-yl.

In some embodiments of the compounds of formula (IA-1), R² is —P(O)R^{4b}R^{4c}, wherein R^{4b} and R^{4c} are as defined above for formula (I).

In some embodiments of the compounds of formula (IA-1), R² is —OR^{4a}, wherein R^{4a} is as defined above for formula (I).

In some embodiments of the compounds of formula (IA-1), R^{4a} is —CH(CH₃), —CH₂CH₂OH, —CH₂C(CH₃)₂OH, cyclopropyl, cyclobutyl, or cyclopentyl.

In some embodiments, the compounds of formula (IA) are compounds of formula (IA-2):

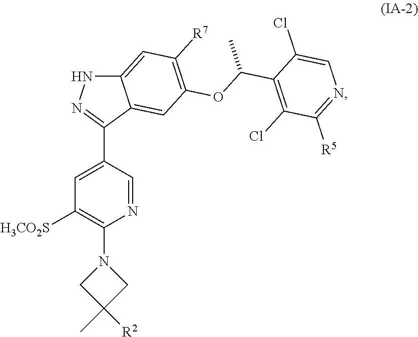

or a pharmaceutically acceptable salt thereof, wherein
   R⁵ is H or CH₃; and
   R⁷ is H, F, or OCH₃; and
   R² is as described above for formula (I).

In some embodiments of the compounds of formula (IA-2), R⁵ is H.

In some embodiments of the compounds of formula (IA-2), R⁵ is CH₃.

In some embodiments of the compounds of formula (IA-2), R⁷ is H.

In some embodiments of the compounds of formula (IA-2), R⁷ is F.

In some embodiments of the compounds of formula (IA-2), R⁷ is OCH₃.

In some embodiments of the compounds of formula (IA-2), R² is optionally substituted C₁-C₆alkyl.

In some embodiments, of the compounds of formula (IA-2), R² is CH₃, —CH₂—NHSO₂(CH₃), —CH₂—N(CH₃)SO₂(CH₃),

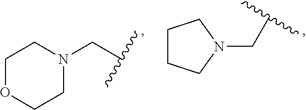

—CH₂—NH(CH₃), —CH₂—NH(—CH(CH₃)₂), —CH₂—N(CH₃)₂, or —CH₂—P(O)(CH₃)₂.

In some embodiments of the compounds of formula (IA-2), R² is —NR³R⁴, wherein R³ and R⁴ are as defined above for formula (I).

In some embodiments of compounds of formula (IA-2), R³ is H.

In some embodiments of compounds of formula (IA-2), R³ is CH₃.

In some embodiments of compounds of formula (IA-2), R⁴ is H.

In some embodiments of compounds of formula (IA-2), R⁴ is CH₃, —CH₂CH₃, —CH(CH₃), —CH₂-cyclopropyl, —CH₂CH₂CHF₂, —CH₂CHF₂, —CH₂CH₂OH, —CH₂C(CH₃)₂OH, —CH₂CHOCH₃, —C(O)CH₂N(CH₃)₂,

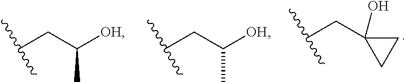

In other embodiments of compounds of formula (IA-2), R⁴ is —CH₂CH(CH₃)₂, —CH₂CH₂CH₃, —CH(CH₃)CH₂CH₃,

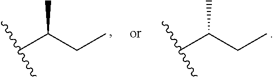

In some embodiments of compounds of formula (IA-2), R⁴ is —CH₂CH═CH₂.

In some embodiments of compounds of formula (IA-2), R⁴ is cyclopropyl, cyclobutyl, or cyclopentyl.

In some embodiments of compounds of formula (IA-2), R⁴ is oxetan-3-yl, thietane-3-yl-1,1-dioxide, tetrahydrofuran-3-yl,

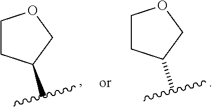 [SMILES: *[C@H]1CCOC1 ; *[C@@H]1CCOC1]

In some embodiments of compounds of formula (IA-2), R⁴ is C(O)CH₂N(CH₃)₂.

In some embodiments of compounds of formula (IA-2), R³ and R⁴, together with the N atom to which they are both attached, form a 3- to 6-membered heterocycloalkyl optionally substituted with one or more substituents that are each C₁-C₆alkyl, C₁-C₆alkoxyl, or OH.

In some embodiments, R³ and R⁴, together with the N atom to which they are both attached, form a 3,3-dimethyl-azetidin-1-yl, 3-hydroxy-3-methylazetidin-1-yl, 1-methyl-4-azaphosphinan-4-yl 1-oxide, 1-methyl-4-azaphosphinan-4-yl 1-oxide, or 3-methoxy-3-methylazetidin-1-yl.

In some embodiments of the compounds of formula (IA-2), R² is —P(O)R^{4b}R^{4c}, wherein R^{4b} and R^{4c} are as defined above for formula (I).

In some embodiments of the compounds of formula (IA-2), R² is —OR^{4a}, wherein R^{4a} is as defined above for formula (I).

In some embodiments of the compounds of formula (IA-2), R^{4a} is —CH(CH₃), —CH₂CH₂OH, —CH₂C(CH₃)₂OH, cyclopropyl, cyclobutyl, or cyclopentyl.

In some embodiments, the compounds of formula (IA) are compounds of formula (IA-3-1):

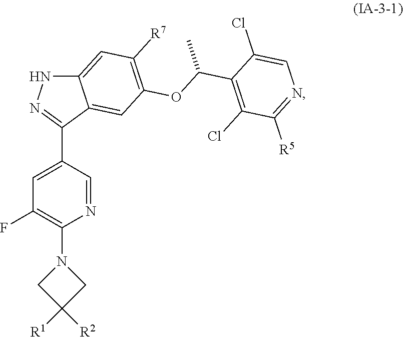

or a pharmaceutically acceptable salt thereof, wherein
   R⁵ is H or CH₃; and
   R⁷ is H, F, or OCH₃; and
   R¹ and R² is as described above for formula (I).

In some embodiments of the compounds of formula (IA-3-1), R⁵ is H.

In some embodiments of the compounds of formula (IA-3-1), R⁵ is CH₃.

In some embodiments of the compounds of formula (IA-3-1), R⁷ is H.

In some embodiments of the compounds of formula (IA-3-1), R⁷ is F.

In some embodiments of the compounds of formula (IA-3-1), R⁷ is OCH₃.

In some embodiments of the compounds of formula (IA-3-1), R¹ is H.

In some embodiments of the compounds of formula (IA-3-1), R¹ is optionally substituted C₁-C₆alkyl, such as, for example, methyl, ethyl, n-propyl, isopropyl, isosbutyl, sec-butyl, —CH₂CH₂OH, —CH₂CH₂OCH₃, —CH₂CH₂F, —CH₂CHF₂, —CH₂CH₂CN, —CH₂CH₂CH₂OH, —CH₂OCH₃, —CH₂OCH(CH₃)₂, —CH₂OCH₂CH₃,

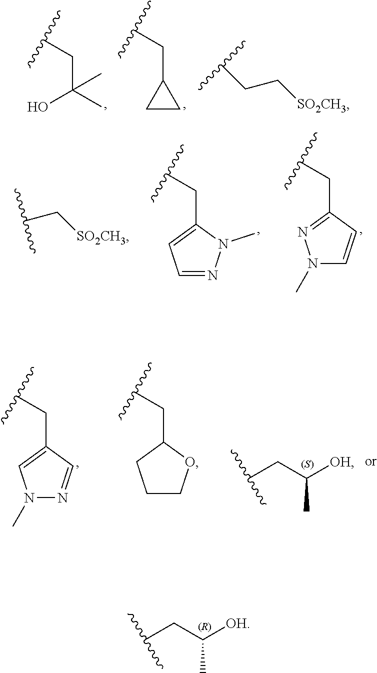

In some embodiments of the compounds of formula (IA-3-1), R¹ is optionally substituted C₂-C₆alkenyl, such as, for example,

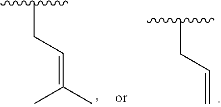

In some embodiments of the compounds of formula (IA-3-1), R¹ is C₃-C₆cycloalkyl, such as, for example, cyclobutyl.

In some embodiments of the compounds of formula (IA-3-1), R¹ is optionally substituted heterocycloalkyl, such as, for example,

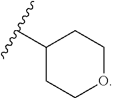 [SMILES: *C1CCOCC1]

In some embodiments of the compounds of formula IA-3-1), R¹ is optionally substituted heteroaryl, such as, for example,

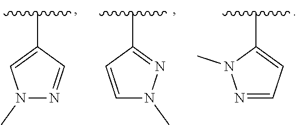

In some embodiments of the compounds of formula (IA-3-1), R¹ is —C(O)OR, such as, for example,

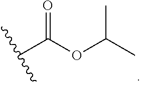 [SMILES: *C(=O)OC(C)C]

In some embodiments of the compounds of formula (IA-3-1), R¹ is —C(O)NR³R⁴, such as, for example,

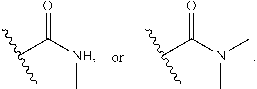

In some embodiments of the compounds of formula (IA-3-1), R² is optionally substituted C₁-C₆alkyl, —NR³R⁴, —OR^{4a}, or —C(O)NR³R⁴.

In some embodiments of the compounds of formula (IA-3-1), R² is —NR³R⁴.

In some embodiments, the compounds of formula (IA) are compounds of formula (IA-3):

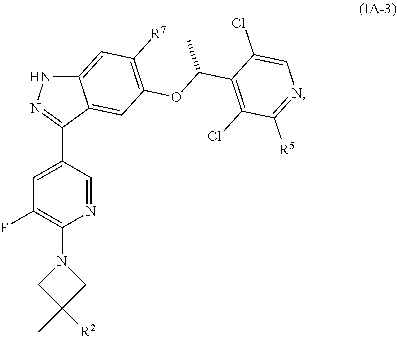

or a pharmaceutically acceptable salt thereof, wherein
   R⁵ is H or CH₃; and
   R⁷ is H, F, or OCH₃; and
   R² is as described above for formula (I).

In some embodiments of the compounds of formula (IA-3), R⁵ is H.

In some embodiments of the compounds of formula (IA-3), R⁵ is CH₃.

In some embodiments of the compounds of formula (IA-3), R⁷ is H.

In some embodiments of the compounds of formula (IA-3), R⁷ is F.

In some embodiments of the compounds of formula (IA-3), R⁷ is OCH₃.

In some embodiments of the compounds of formula (IA-3), R² is optionally substituted C₁-C₆alkyl.

In some embodiments, of the compounds of formula (IA-3), R² is CH₃, —CH₂—NHSO₂(CH₃), —CH₂—N(CH₃)SO₂(CH₃),

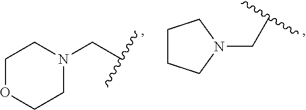

—CH₂—NH(CH₃), —CH₂—NH(—CH(CH₃)₂), —CH₂—N(CH₃)₂, or —CH₂—P(O)(CH₃)₂.

In some embodiments of the compounds of formula (IA-3), R² is —NR³R⁴, wherein R³ and R⁴ are as defined above for formula (I).

In some embodiments of compounds of formula (IA-3), R³ is H.

In some embodiments of compounds of formula (IA-3), R³ is CH₃.

In some embodiments of compounds of formula (IA-3), R⁴ is H.

In some embodiments of compounds of formula (IA-3), R⁴ is CH₃, —CH₂CH₃, —CH(CH₃), —CH₂-cyclopropyl, —CH₂CH₂CHF₂, —CH₂CHF₂, —CH₂CH₂OH, —CH₂C(CH₃)₂OH, —CH₂CHOCH₃, —C(O)CH₂N(CH₃)₂,

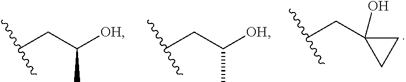

In other embodiments of compounds of formula (IA-3), R⁴ is CH₂CH(CH₃)₂, —CH₂CH₂CH₃, —CH(CH₃)CH₂CH₃,

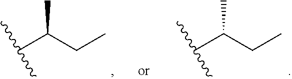

In some embodiments of compounds of formula (IA-3), R⁴ is —CH₂CH═CH₂.

In some embodiments of compounds of formula (IA-3), R⁴ is cyclopropyl, cyclobutyl, or cyclopentyl.

In some embodiments of compounds of formula (IA-3), R⁴ is oxetan-3-yl, thietane-3-yl-1,1-dioxide, tetrahydrofuran-3-yl,

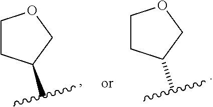 [SMILES: *[C@H]1CCOC1 ; *[C@@H]1CCOC1]

In some embodiments of compounds of formula (IA-3), R⁴ is C(O)CH₂N(CH₃)₂.

In some embodiments of compounds of formula (IA-3), R³ and R⁴, together with the N atom to which they are both attached, form a 3- to 6-membered heterocycloalkyl optionally substituted with one or more substituents that are each C₁-C₆alkyl, C₁-C₆alkoxyl, or OH.

In some embodiments, R³ and R⁴, together with the N atom to which they are both attached, form a 3,3-dimethyl-azetidin-1-yl, 3-hydroxy-3-methylazetidin-1-yl, 1-methyl-4-azaphosphinan-4-yl 1-oxide, 1-methyl-4-azaphosphinan-4-yl 1-oxide, or 3-methoxy-3-methylazetidin-1-yl.

In some embodiments of the compounds of formula (IA-3), R² is —P(O)R^{4b}R^{4c}, wherein R^{4b} and R^{4c} are as defined above for formula (I).

In some embodiments of the compounds of formula (IA-3), R² is —OR^{4a}, wherein R^{4a} is as defined above for formula (I).

In some embodiments of the compounds of formula (IA-3), R^{4a} is —CH(CH₃), —CH₂CH₂OH, —CH₂C(CH₃)₂OH, cyclopropyl, cyclobutyl, or cyclopentyl.

In some embodiments, the compounds of formula (IA) are compounds of formula (IA-4-1):

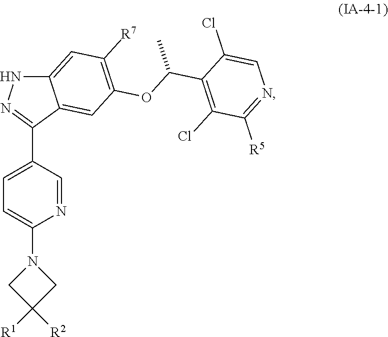

or a pharmaceutically acceptable salt thereof, wherein
   R⁵ is H or CH₃; and
   R⁷ is H, F, or OCH₃; and
   R¹ and R² is as described above for formula (I).

In some embodiments of the compounds of formula (IA-4-1), R⁵ is H.

In some embodiments of the compounds of formula (IA-4-1), R⁵ is CH₃.

In some embodiments of the compounds of formula (IA-4-1), R⁷ is H.

In some embodiments of the compounds of formula (IA-4-1), R⁷ is F.

In some embodiments of the compounds of formula (IA-4-1), R⁷ is OCH₃.

In some embodiments of the compounds of formula (IA-4-1), R¹ is H.

In some embodiments of the compounds of formula (IA-4-1), R¹ is optionally substituted C₁-C₆alkyl, such as, for example, methyl, ethyl, n-propyl, isopropyl, isosbutyl, sec-butyl, —CH₂CH₂OH, —CH₂CH₂OCH₃, —CH₂CH₂F, —CH₂CHF₂, —CH₂CH₂CN, —CH₂CH₂CH₂OH, —CH₂OCH₃, —CH₂OCH(CH₃)₂, —CH₂OCH₂CH₃,

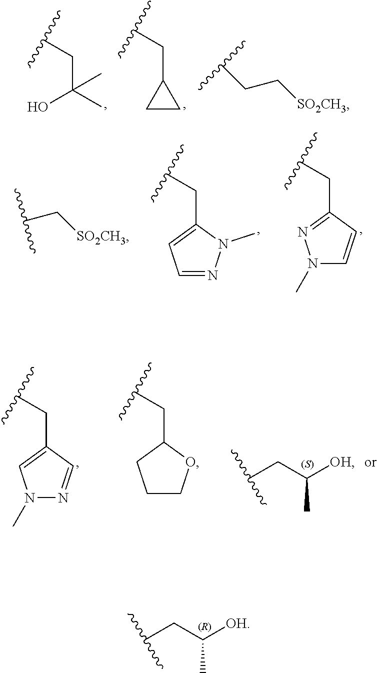

In some embodiments of the compounds of formula (IA-4-1), R¹ is optionally substituted C₂-C₆alkenyl, such as, for example,

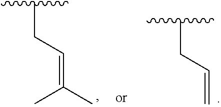

In some embodiments of the compounds of formula (IA-4-1), R¹ is optionally substituted heteroaryl, such as, for example,

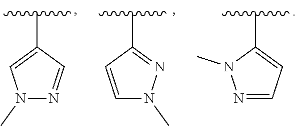

In some embodiments of the compounds of formula (IA-4-1), R¹ is —C(O)OR³, such as, for example,

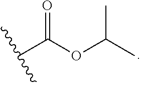 [SMILES: *C(=O)OC(C)C]

In some embodiments of the compounds of formula (IA-4-1), R¹ is —C(O)NR³R⁴, such as, for example,

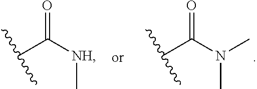

In some embodiments of the compounds of formula (IA-4-1), R² is optionally substituted C₁-C₆alkyl, —NR³R⁴, or —OR^{4a}.

In some embodiments of the compounds of formula (IA-4-1), R² is —NR³R⁴.

In some embodiments of the compounds of formula (IA-4-1), R² is optionally substituted C₁-C₆alkyl.

In some embodiments, the compounds of formula (IA) are compounds of formula (IA-4):

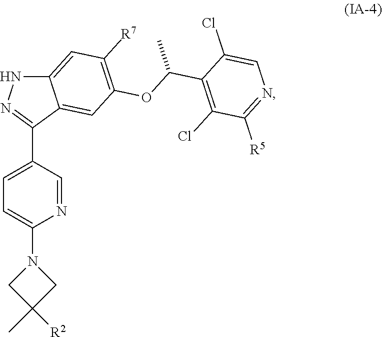

or a pharmaceutically acceptable salt thereof, wherein
   R⁵ is H or CH₃; and
   R⁷ is H, F, or OCH₃; and
   R² is as described above for formula (I).

In some embodiments of the compounds of formula (IA-4), R⁵ is H.

In some embodiments of the compounds of formula (IA-4), R⁵ is CH₃.

In some embodiments of the compounds of formula (IA-4), R⁷ is H.

In some embodiments of the compounds of formula (IA-4), R⁷ is F.

In some embodiments of the compounds of formula (IA-4), R⁷ is OCH₃.

In some embodiments of the compounds of formula (IA-4), R² is optionally substituted C₁-C₆alkyl.

In some embodiments, of the compounds of formula (IA-4), R² is CH₃, —CH₂—NHSO₂(CH₃), —CH₂—N(CH₃)SO₂(CH₃),

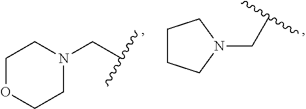

—CH₂—NH(CH₃), —CH₂—NH(—CH(CH₃)₂), —CH₂—N(CH₃)₂, or —CH₂—P(O)(CH₃)₂.

In some embodiments of the compounds of formula (IA-4), R² is —NR³R⁴, wherein R³ and R⁴ are as defined above for formula (I).

In some embodiments of compounds of formula (IA-4), R³ is H.

In some embodiments of compounds of formula (IA-4), R³ is CH₃.

In some embodiments of compounds of formula (IA-4), R⁴ is H.

In some embodiments of compounds of formula (IA-4), R⁴ is CH₃, —CH₂CH₃, —CH(CH₃), —CH₂-cyclopropyl, —CH₂CH₂CHF₂, —CH₂CHF₂, —CH₂CH₂OH, —CH₂C(CH₃)₂OH, —CH₂CHOCH₃, —C(O)CH₂N(CH₃)₂,

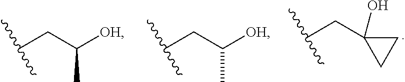

In other embodiments of compounds of formula (IA-4), R⁴ is —CH₂CH(CH₃)₂, —CH₂CH₂CH₃, —CH(CH₃)CH₂CH₃,

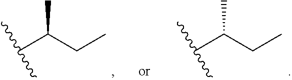

In some embodiments of compounds of formula (IA-4), R⁴ is —CH₂CH═CH₂.

In some embodiments of compounds of formula (IA-4), R⁴ is cyclopropyl, cyclobutyl, or cyclopentyl.

In some embodiments of compounds of formula (IA-4), R⁴ is oxetan-3-yl, thietane-3-yl-1,1-dioxide, tetrahydrofuran-3-yl,

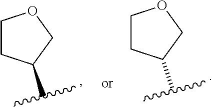 [SMILES: *[C@H]1CCOC1 ; *[C@@H]1CCOC1]

In some embodiments of compounds of formula (IA-4), R⁴ is C(O)CH₂N(CH₃)₂.

In some embodiments of compounds of formula (IA-4), R³ and R⁴, together with the N atom to which they are both attached, form a 3- to 6-membered heterocycloalkyl optionally substituted with one or more substituents that are each C₁-C₆alkyl, C₁-C₆alkoxyl, or OH.

In some embodiments, R³ and R⁴, together with the N atom to which they are both attached, form a 3,3-dimethyl-azetidin-1-yl, 3-hydroxy-3-methylazetidin-1-yl, 1-methyl-4-azaphosphinan-4-yl 1-oxide, 1-methyl-4-azaphosphinan-4-yl 1-oxide, or 3-methoxy-3-methylazetidin-1-yl.

In some embodiments of the compounds of formula (IA-4), R² is —P(O)R^{4b}R^{4c}, wherein R^{4b} and R^{4c} are as defined above for formula (I).

In some embodiments of the compounds of formula (IA-4), R² is —OR^{4a}, wherein R^{4a} is as defined above for formula (I).

In some embodiments of the compounds of formula (IA-4), R^{4a} is —CH(CH₃), —CH₂CH₂OH, —CH₂C(CH₃)₂OH, cyclopropyl, cyclobutyl, or cyclopentyl.

In some embodiments, the compounds of formula (IA) are compounds of formula (IA-5):

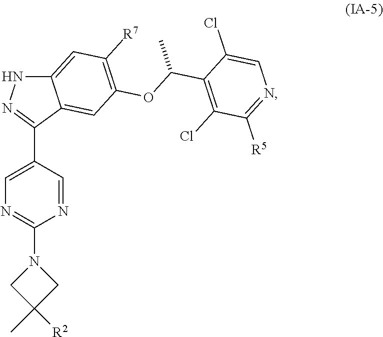

or a pharmaceutically acceptable salt thereof, wherein
   R⁵ is H or CH₃; and
   R⁷ is H, F, or OCH₃; and
   R² is as described above for formula (I).

In some embodiments of the compounds of formula (IA-5), R⁵ is H.

In some embodiments of the compounds of formula (IA-5), R⁵ is CH₃.

In some embodiments of the compounds of formula (IA-5), R⁷ is H.

In some embodiments of the compounds of formula (IA-5), R⁷ is F.

In some embodiments of the compounds of formula (IA-5), R⁷ is OCH₃.

In some embodiments of the compounds of formula (IA-5), R² is optionally substituted C₁-C₆alkyl.

In some embodiments, of the compounds of formula (IA-5), R² is CH₃, —CH₂—NHSO₂(CH₃), —CH₂—N(CH₃)SO₂(CH₃),

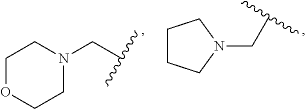

CH₂—NH(CH₃), —CH₂—NH(—CH(CH₃)₂), —CH₂—N(CH₃)₂, or —CH₂—P(O)(CH₃)₂.

In some embodiments of the compounds of formula (IA-5), R² is —NR³R⁴, wherein R³ and R⁴ are as defined above for formula (I).

In some embodiments of compounds of formula (IA-5), R³ is H.

In some embodiments of compounds of formula (IA-5), R³ is CH₃.

In some embodiments of compounds of formula (IA-5), R⁴ is H.

In some embodiments of compounds of formula (IA-5), R⁴ is CH₃, —CH₂CH₃, —CH(CH₃), —CH₂-cyclopropyl, —CH₂CH₂CHF₂, —CH₂CHF₂, —CH₂CH₂OH, —CH₂C(CH₃)₂OH, —CH₂CHOCH₃, —C(O)CH₂N(CH₃)₂,

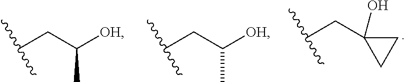

In other embodiments of compounds of formula (IA-5), R⁴ is —CH₂CH(CH₃)₂, —CH₂CH₂CH₃, —CH(CH₃)CH₂CH₃,

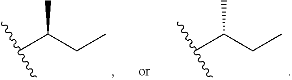

In some embodiments of compounds of formula (IA-5), R⁴ is —CH₂CH═CH₂.

In some embodiments of compounds of formula (IA-5), R⁴ is cyclopropyl, cyclobutyl, or cyclopentyl.

In some embodiments of compounds of formula (IA-5), R⁴ is oxetan-3-yl, thietane-3-yl-1,1-dioxide, tetrahydrofuran-3-yl,

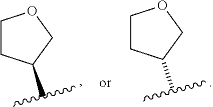 [SMILES: *[C@H]1CCOC1 ; *[C@@H]1CCOC1]

In some embodiments of compounds of formula (IA-5), R⁴ is C(O)CH₂N(CH₃)₂.

In some embodiments of compounds of formula (IA-5), R³ and R⁴, together with the N atom to which they are both attached, form a 3- to 6-membered heterocycloalkyl optionally substituted with one or more substituents that are each C₁-C₆alkyl, C₁-C₆alkoxyl, or OH.

In some embodiments, R³ and R⁴, together with the N atom to which they are both attached, form a 3,3-dimethyl-azetidin-1-yl, 3-hydroxy-3-methylazetidin-1-yl, 1-methyl-4-azaphosphinan-4-yl 1-oxide, 1-methyl-4-azaphosphinan-4-yl 1-oxide, or 3-methoxy-3-methylazetidin-1-yl.

In some embodiments of the compounds of formula (IA-5), R² is —P(O)R^{4b}R^{4c}, wherein R^{4b} and R^{4c} are as defined above for formula (I).

In some embodiments of the compounds of formula (IA-5), R² is —OR^{4a}, wherein R^{4a} is as defined above for formula (I).

In some embodiments of the compounds of formula (IA-5), R^{4a} is —CH(CH₃), —CH₂CH₂OH, —CH₂C(CH₃)₂OH, cyclopropyl, cyclobutyl, or cyclopentyl.

In some embodiments, the compounds of formula (IA) are compounds of formula (IA-6):

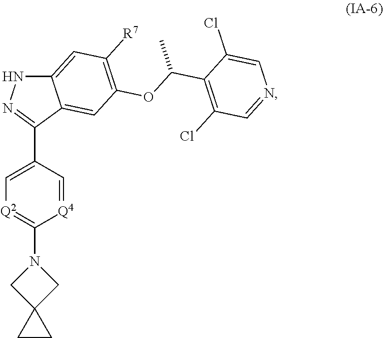

or a pharmaceutically acceptable salt thereof.

In some aspects, the disclosure is directed to the compounds of formula (I) that are compounds of formula (IB):

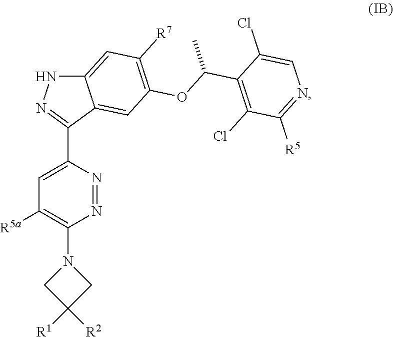

or a pharmaceutically acceptable salt thereof, wherein
   R^{5a} is H, F, or —CN;
   R⁵ is H or CH₃; and
   R⁷ is H, F, or OCH₃,
and R¹ and R² are as described above for formula (I).

In some embodiments of the compounds of formula (IB), R⁵ is H.

In some embodiments of the compounds of formula (IB), R⁵ is CH₃.

In some embodiments of the compounds of formula (IB), R⁷ is H.

In some embodiments of the compounds of formula (IB), R⁷ is F.

In some embodiments of the compounds of formula (IB), R⁷ is OCH₃.

In some embodiments of the compounds of formula (IB), R^{5a} is H or F.

In some embodiments of the compounds of formula (IB), R^{5a} is H.

In some embodiments of the compounds of formula (IB), R^{5a} is F.

In some embodiments of the compounds of formula (IB), R¹ is H.

In some embodiments of the compounds of formula (IB), R¹ is optionally substituted C₁-C₆alkyl, such as, for example, methyl, ethyl, n-propyl, isopropyl, isosbutyl, sec-butyl, —CH₂CH₂OH, —CH₂CH₂OCH₃, —CH₂CH₂F, —CH₂CHF₂, —CH₂CH₂CN, —CH₂CH₂CH₂OH, —CH₂OCH₃, —CH₂OCH(CH₃)₂, —CH₂OCH₂CH₃,

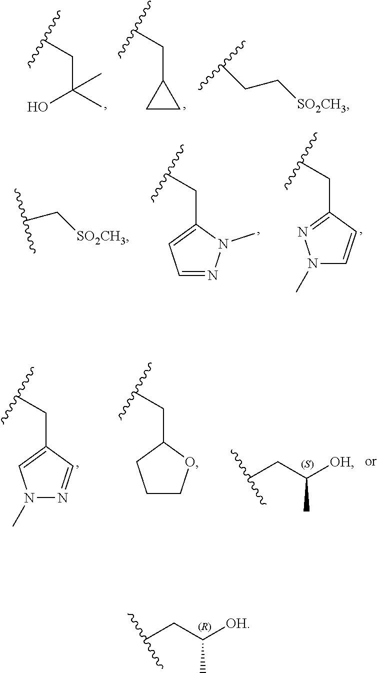

In some embodiments of the compounds of formula (IB), R¹ is optionally substituted C₂-C₆alkenyl, such as, for example,

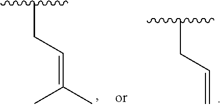

In some embodiments of the compounds of formula (IB), R¹ is optionally substituted heteroaryl, such as, for example,

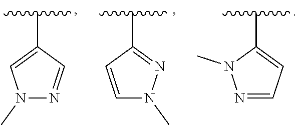

In some embodiments of the compounds of formula (IB), R¹ is —C(O)OR³, such as, for example,

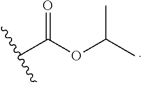 [SMILES: *C(=O)OC(C)C]

In some embodiments of the compounds of formula (TB), R¹ is —C(O)NR³R⁴, such as, for example,

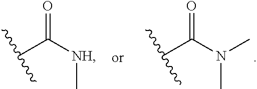

In some embodiments of the compounds of formula (IB), R² is optionally substituted C₁-C₆alkyl, —NR³R⁴, or —OR^{4a}.

In some embodiments of the compounds of formula (IB), R² is —NR³R⁴.

In some embodiments of the compounds of formula (B), R² is OR^{4a}.

In some embodiments, the compounds of the disclosure are the compounds of Examples 1-153 described herein, or pharmaceutically acceptable salts thereof.

In other embodiments, the compounds of the disclosure are the compounds of Examples 154-449 described herein, or pharmaceutically acceptable salts thereof.

In other embodiments, the compounds of the disclosure are the compounds shown in the following tables, or pharmaceutically acceptable salts thereof:

| | |
|---|---|
| 1-[5-[5-[(1R)-1-(3,5- dichloro-4- pyridyl)ethoxy]-1H- indazol-3-yl]-3- fluoro-2-pyridyl]-N- (2,2-difluoroethyl)- 3-methyl-azetidin-3- amine | 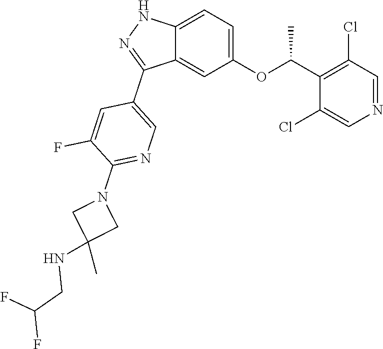 [SMILES: C[C@@H](Oc1ccc2[nH]nc(-c3cnc(N4CC(C)(NCC(F)F)C4)c(F)c3)c2c1)c1c(Cl)cncc1Cl] |
| 1-[1-[5-[5-[(1R)-1- (3,5-dichloro-4- pyridyl)ethoxy]-1H- indazol-3-yl]-2- pyridyl]-3-methyl- azetidin-3-yl]-3- methyl-azetidin-3-ol | 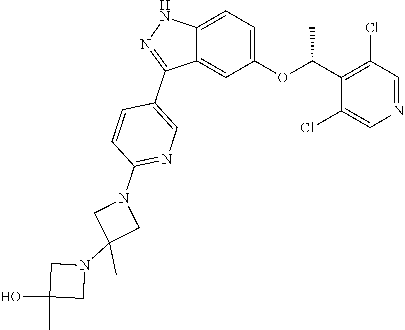 [SMILES: C[C@@H](Oc1ccc2[nH]nc(-c3ccc(N4CC(C)(N5CC(C)(O)C5)C4)nc3)c2c1)c1c(Cl)cncc1Cl] |
| 4-[1-[5-[5-[(1R)-1- (3,5-dichloro-4- pyridyl)ethoxy]-6- fluoro-1H-indazol-3- yl]-3-fluoro-2- pyridyl]-3-methyl- azetidin-3- yl]morpholine | 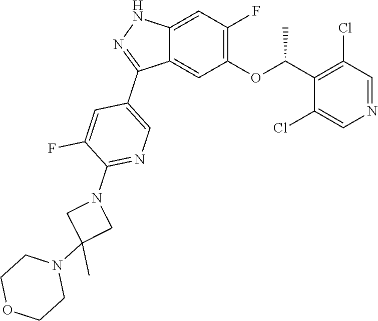 [SMILES: C[C@@H](Oc1cc2c(-c3cnc(N4CC(C)(N5CCOCC5)C4)c(F)c3)n[nH]c2cc1F)c1c(Cl)cncc1Cl] |
| 3-[6-[3-(azetidin-1- yl)-3-methyl- azetidin-1-yl]-5- fluoro-3-pyridyl]-5- [(1R)-1-(3,5- dichloro-4- pyridyl)ethoxy]-6- fluoro-1H-indazole | 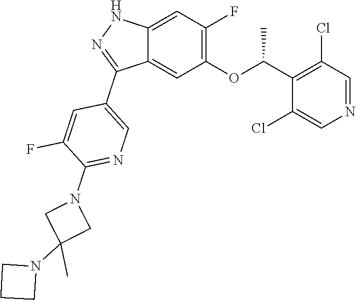 [SMILES: C[C@@H](Oc1cc2c(-c3cnc(N4CC(C)(N5CCC5)C4)c(F)c3)n[nH]c2cc1F)c1c(Cl)cncc1Cl] |
| 2-[[1-[5-[5-[(1R)-1- (3,5-dichloro-4- pyridyl)ethoxy]-6- fluoro-1H-indazol-3- yl]-3-fluoro-2- pyridyl]-3-methyl- azetidin-3- yl]amino]ethanol | 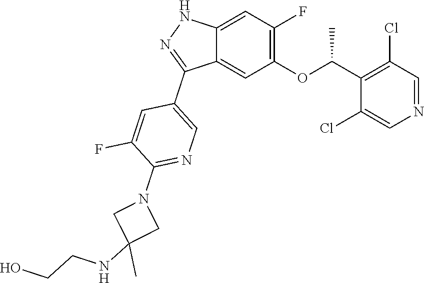 [SMILES: C[C@@H](Oc1cc2c(-c3cnc(N4CC(C)(NCCO)C4)c(F)c3)n[nH]c2cc1F)c1c(Cl)cncc1Cl] |
| 1-[1-[5-[5-[(1R)-1- (3,5-dichloro-4- pyridyl)ethoxy]-6- fluoro-1H-indazol-3- yl]-3-fluoro-2- pyridyl]-3-methyl- azetidin-3-yl]-3- methyl-azetidin-3-ol | 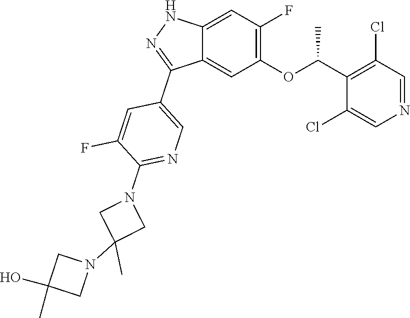 [SMILES: C[C@@H](Oc1cc2c(-c3cnc(N4CC(C)(N5CC(C)(O)C5)C4)c(F)c3)n[nH]c2cc1F)c1c(Cl)cncc1Cl] |
| 5-[5-[(1R)-1-(3,5- dichloro-4- pyridyl)ethoxy]-1H- indazol-3-yl]-2-[3- methyl-3-(4-methyl- 4-oxo-1,4λ⁵- azaphosphinan-1- yl)azetidin-1- yl]pyridine-3- carbonitrile | 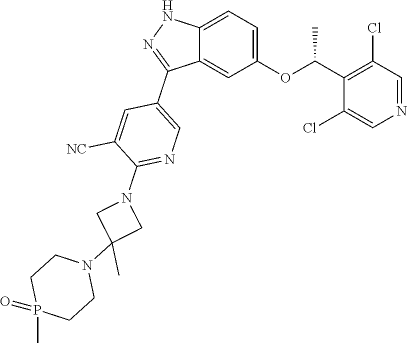 [SMILES: C[C@@H](Oc1ccc2[nH]nc(-c3cnc(N4CC(C)(N5CCP(C)(=O)CC5)C4)c(C#N)c3)c2c1)c1c(Cl)cncc1Cl] |
| 1-[1-[5-[5-[(1R)-1- (3,5-dichloro-4- pyridyl)ethoxy]-1H- indazol-3-yl]-3- fluoro-2-pyridyl]-3- methyl-azetidin-3- yl]-4-methyl-1,4λ⁵- azaphosphinane 4- oxide | 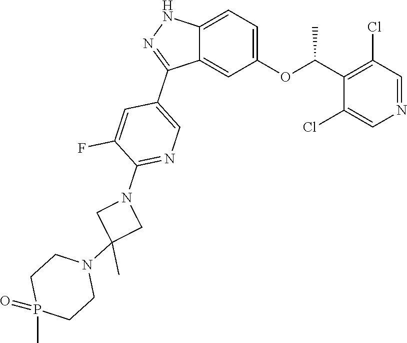 [SMILES: C[C@@H](Oc1ccc2[nH]nc(-c3cnc(N4CC(C)(N5CCP(C)(=O)CC5)C4)c(F)c3)c2c1)c1c(Cl)cncc1Cl] |
| 2-[3- (cyclopropylmethyl- amino)-3-methyl- azetidin-1-yl]-5-[5- [(1R)-1-(3,5- dichloro-4- pyridyl)ethoxy]-4- fluoro-1H-indazol-3- yl]pyridine-3- carbonitrile | 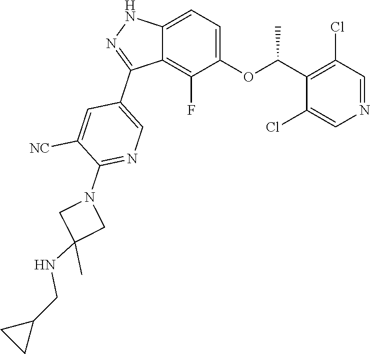 [SMILES: C[C@@H](Oc1ccc2[nH]nc(-c3cnc(N4CC(C)(NCC5CC5)C4)c(C#N)c3)c2c1F)c1c(Cl)cncc1Cl] |
| 5-[5-[(1R)-1-(3,5- dichloro-4- pyridyl)ethoxy]-4- fluoro-1H-indazol-3- yl]-2-(3-methyl-3- pyrrolidin-1-yl- azetidin-1- yl)pyridine-3- carbonitrile | 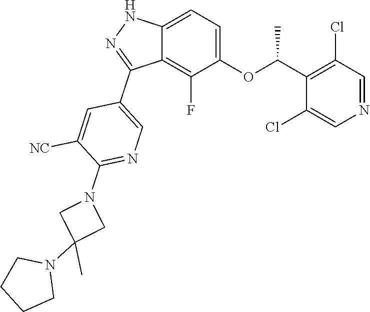 [SMILES: C[C@@H](Oc1ccc2[nH]nc(-c3cnc(N4CC(C)(N5CCCC5)C4)c(C#N)c3)c2c1F)c1c(Cl)cncc1Cl] |
| 5-[5-[(1R)-1-(3,5- dichloro-4- pyridyl)ethoxy]-4- fluoro-1H-indazol-3- yl]-2-[3- (isopropylamino)-3- methyl-azetidin-1- yl]pyridine-3- carbonitrile | 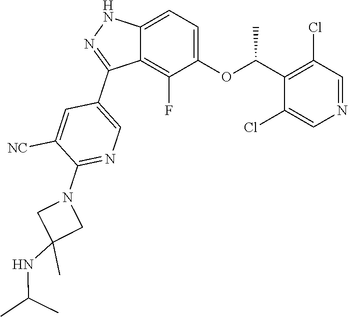 [SMILES: CC(C)NC1(C)CN(c2ncc(-c3n[nH]c4ccc(O[C@H](C)c5c(Cl)cncc5Cl)c(F)c34)cc2C#N)C1] |
| 5-[5-[(1R)-1-(3,5- dichloro-4- pyridyl)ethoxy]-4- fluoro-1H-indazol-3- yl]-2-(3-methyl-3- morpholino- azetidin-1- yl)pyridine-3- carbonitrile | 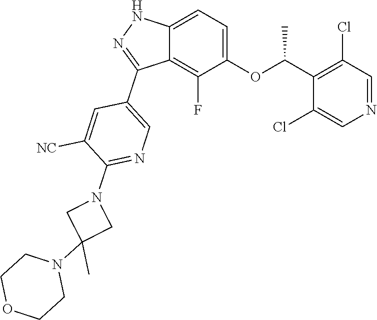 [SMILES: C[C@@H](Oc1ccc2[nH]nc(-c3cnc(N4CC(C)(N5CCOCC5)C4)c(C#N)c3)c2c1F)c1c(Cl)cncc1Cl] |
| 5-[5-[(1R)-1-(3,5- dichloro-4- pyridyl)ethoxy]-4- fluoro-1H-indazol-3- yl]-2-[3- (dimethylphosphoryl- methyl)-3-methyl- azetidin-1- yl]pyridine-3- carbonitrile | 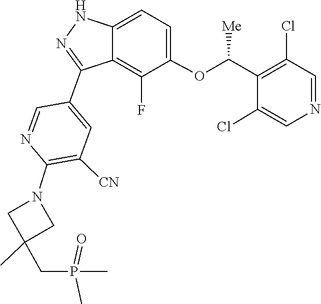 [SMILES: C[C@@H](Oc1ccc2[nH]nc(-c3cnc(N4CC(C)(CP(C)(C)=O)C4)c(C#N)c3)c2c1F)c1c(Cl)cncc1Cl] |
| 5-[(1R)-1-(3,5- dichloro-4- pyridyl)ethoxy]-3- [6-[3- (dimethylphosphoryl methyl)-3-methyl- azetidin-1-yl]-5- fluoro-3-pyridyl]-4- fluoro-1H-indazole | 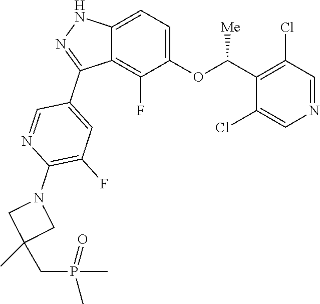 [SMILES: C[C@@H](Oc1ccc2[nH]nc(-c3cnc(N4CC(C)(CP(C)(C)=O)C4)c(F)c3)c2c1F)c1c(Cl)cncc1Cl] |
| 4-[1-[5-[5-[(1R)-1- (3,5-dichloro-4- pyridyl)ethoxy]-4- fluoro-1H-indazol-3- yl]-3-fluoro-2- pyridyl]-3-methyl- azetidin-3- yl]morpholine | 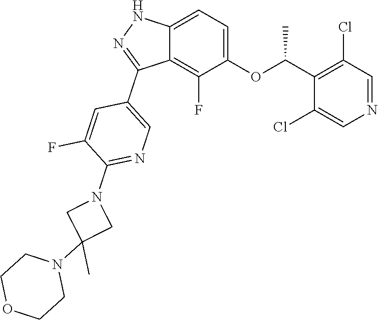 [SMILES: C[C@@H](Oc1ccc2[nH]nc(-c3cnc(N4CC(C)(N5CCOCC5)C4)c(F)c3)c2c1F)c1c(Cl)cncc1Cl] |
| 5-[(1R)-1-(3,5- dichloro-4- pyridyl)ethoxy]-4- fluoro-3-[5-fluoro-6- (3-methyl-3- pyrrolidin-1-yl- azetidin-1-yl)-3- pyridyl]-1H- indazole | 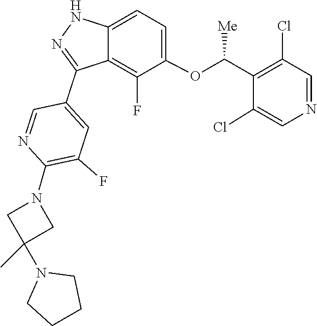 [SMILES: CC1(N2CCCC2)CN(c2ncc(-c3n[nH]c4ccc(OCc5c(Cl)cncc5Cl)c(F)c34)cc2F)C1 ; "Me"] |
| N- (cyclopropylmethyl)- 1-[5-[5-[(1R)-1- (3,5-dichloro-4- pyridyl)ethoxy]-4- fluoro-1H-indazol-3- yl]-3-fluoro-2- pyridyl]-3-methyl- azetidin-3-amine | 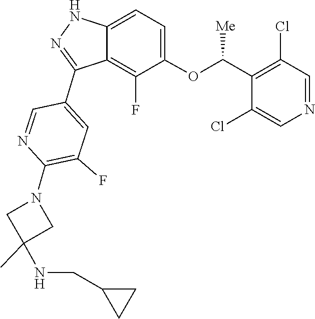 [SMILES: CC1(NCC2CC2)CN(c2ncc(-c3n[nH]c4ccc(OCc5c(Cl)cncc5Cl)c(F)c34)cc2F)C1 ; "Me"] |
| 3-[6-[3-(azetidin-1- yl)-3-methyl- azetidin-1-yl]-5- fluoro-3-pyridyl]-5- [(1R)-1-(3,5- dichloro-4- pyridyl)ethoxy]-4- fluoro-1H-indazole | 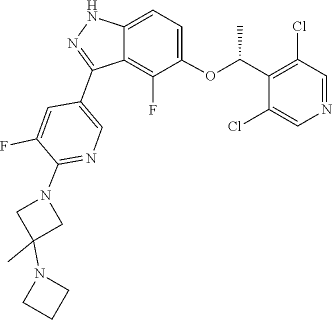 [SMILES: C[C@@H](Oc1ccc2[nH]nc(-c3cnc(N4CC(C)(N5CCC5)C4)c(F)c3)c2c1F)c1c(Cl)cncc1Cl] |
| 5-[(1R)-1-(3,5- dichloro-4- pyridyl)ethoxy]-4- fluoro-3-[5-fluoro-6- [3-methyl-3- (pyrrolidin-1- ylmethyl)azetidin-1- yl]-3-pyridyl]-1H- indazole | 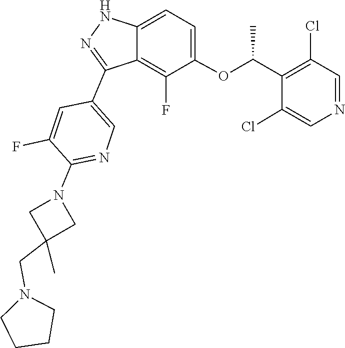 [SMILES: C[C@@H](Oc1ccc2[nH]nc(-c3cnc(N4CC(C)(CN5CCCC5)C4)c(F)c3)c2c1F)c1c(Cl)cncc1Cl] |
| N-[1-[5-[5-[(1R)-1- (3,5-dichloro-4- pyridyl)ethoxy]-1H- indazol-3-yl]-3- fluoro-2-pyridyl]-3- methyl-azetidin-3- yl]-2- (dimethylamino)- acetamide | 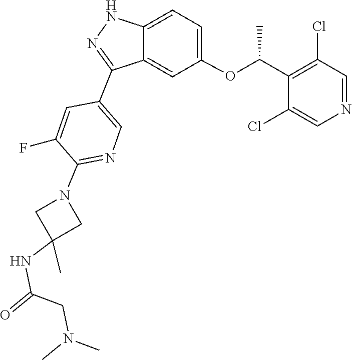 [SMILES: C[C@@H](Oc1ccc2[nH]nc(-c3cnc(N4CC(C)(NC(=O)CN(C)C)C4)c(F)c3)c2c1)c1c(Cl)cncc1Cl] |
| 5-[5-[(1R)-1-(3,5- dichloro-4- pyridyl)ethoxy]-1H- indazol-3-yl]-2-[3- (1,1-dioxo-1,4- thiazinan-4-yl)-3- methyl-azetidin-1- yl]pyridine-3- carbonitrile | 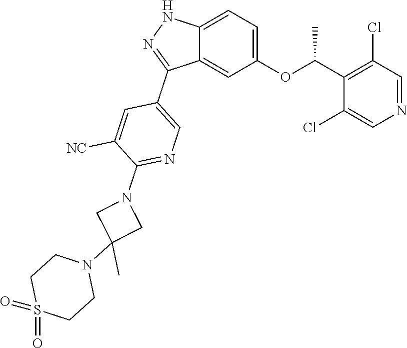 [SMILES: C[C@@H](Oc1ccc2[nH]nc(-c3cnc(N4CC(C)(N5CCS(=O)(=O)CC5)C4)c(C#N)c3)c2c1)c1c(Cl)cncc1Cl] |
| 4-[1-[5-[5-[(1R)-1- (3,5-dichloro-4- pyridyl)ethoxy]-1H- indazol-3-yl]-3- fluoro-2-pyridyl]-3- methyl-azetidin-3- yl]-1,4-thiazinane 1,1-dioxide | 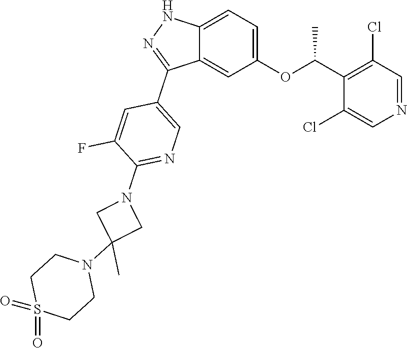 [SMILES: C[C@@H](Oc1ccc2[nH]nc(-c3cnc(N4CC(C)(N5CCS(=O)(=O)CC5)C4)c(F)c3)c2c1)c1c(Cl)cncc1Cl] |
| 5-[5-[(1R)-1-(3,5- dichloro-4- pyridyl)ethoxy]-4- fluoro-1H-indazol-3- yl]-2-[3-(2- hydroxyethylamino)- 3-methyl-azetidin- 1-yl]pyridine-3- carbonitrile | 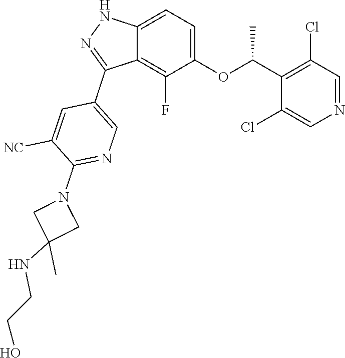 [SMILES: C[C@@H](Oc1ccc2[nH]nc(-c3cnc(N4CC(C)(NCCO)C4)c(C#N)c3)c2c1F)c1c(Cl)cncc1Cl] |
| 2-[[1-[5-[5-[(1R)-1- (3,5-dichloro-4- pyridyl)ethoxy]-4- fluoro-1H-indazol-3- yl]-3-fluoro-2- pyridyl]-3-methyl- azetidin-3- yl]amino]ethanol | 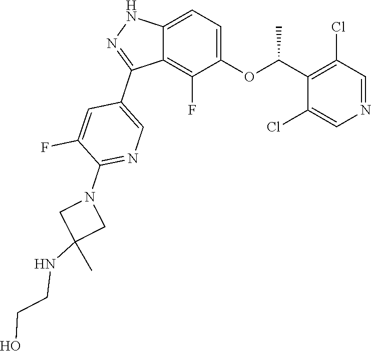 [SMILES: C[C@@H](Oc1ccc2[nH]nc(-c3cnc(N4CC(C)(NCCO)C4)c(F)c3)c2c1F)c1c(Cl)cncc1Cl] |
| 2-[[1-[5-[5-[(1R)-1- (3,5-dichloro-4- pyridyl)ethoxy]-6- methoxy-1H- indazol-3-yl]-3- fluoro-2-pyridyl]-3- methyl-azetidin-3- yl]amino]ethanol | 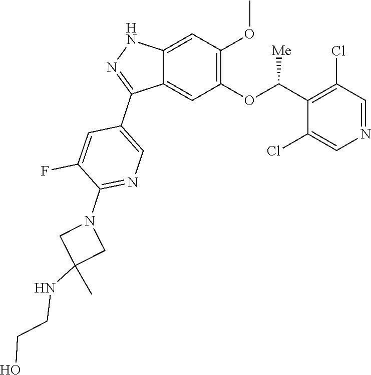 [SMILES: COc1cc2[nH]nc(-c3cnc(N4CC(C)(NCCO)C4)c(F)c3)c2cc1O[C@H](C)c1c(Cl)cncc1Cl] |
| (2R)-1-[[1-[5-[5- [(1R)-1-(3,5- dichloro-4- pyridyl)ethoxy]-6- methoxy-1H- indazol-3-yl]-3- fluoro-2-pyridyl]-3- methyl-azetidin-3- yl]amino]propan-2- ol | 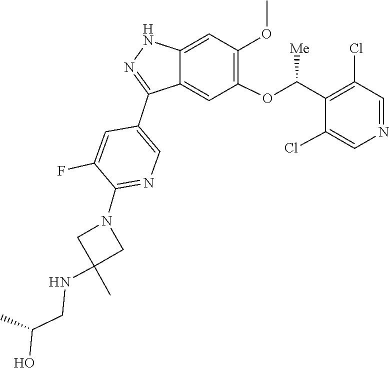 [SMILES: COc1cc2[nH]nc(-c3cnc(N4CC(C)(NC[C@@H](C)O)C4)c(F)c3)c2cc1O[C@H](C)c1c(Cl)cncc1Cl] |
| (2S)-1-[[1-[5-[5- [(1R)-1-(3,5- dichloro-4- pyridyl)ethoxy]-6- methoxy-1H- indazol-3-yl]-3- fluoro-2-pyridyl]-3- methyl-azetidin-3- yl]amino]propan-2- ol | 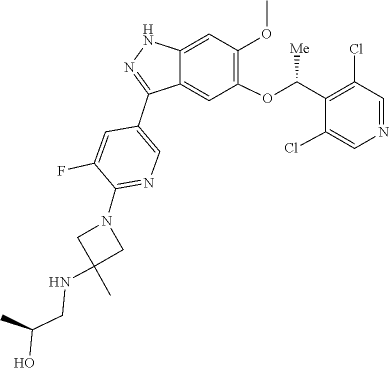 [SMILES: COc1cc2[nH]nc(-c3cnc(N4CC(C)(NC[C@H](C)O)C4)c(F)c3)c2cc1O[C@H](C)c1c(Cl)cncc1Cl] |
| 1-[[1-[5-[5-[(1R)-1- (3,5-dichloro-4- pyridyl)ethoxy]-6- methoxy-1H- indazol-3-yl]-3- fluoro-2-pyridyl]-3- methyl-azetidin-3- yl]amino]-2-methyl- propan-2-ol | 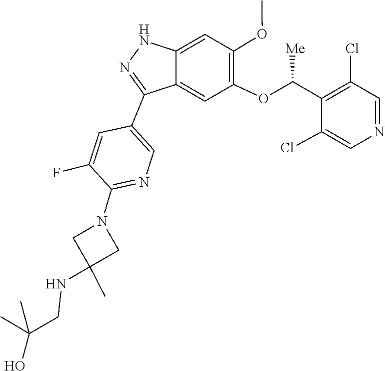 [SMILES: COc1cc2[nH]nc(-c3cnc(N4CC(C)(NCC(C)(C)O)C4)c(F)c3)c2cc1O[C@H](C)c1c(Cl)cncc1Cl] |
| ADE-661 1-[5-[5-[(1R)-1-(3,5- dichloro-4- pyridyl)ethoxy]-1H- indazol-3-yl]-3- fluoro-2-pyridyl]-3- methyl-N-(2- methylsulfonylethyl) azetidin-3-amine | 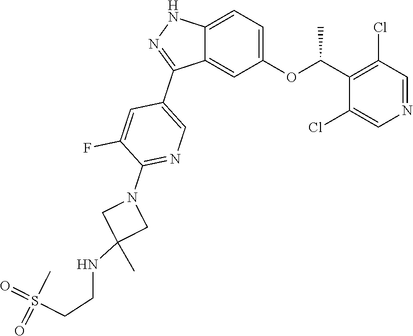 [SMILES: C[C@@H](Oc1ccc2[nH]nc(-c3cnc(N4CC(C)(NCCS(C)(=O)=O)C4)c(F)c3)c2c1)c1c(Cl)cncc1Cl] |
| 5-[5-[(1R)-1-(3,5- dichloro-4- pyridyl)ethoxy]-1H- indazol-3-yl]-2-[3- methyl-3-(2- methylsulfonylethyl amino)azetidin-1- yl]pyridine-3- carbonitrile | 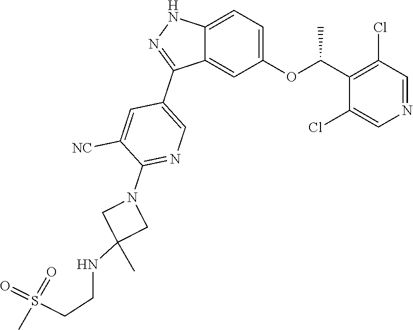 [SMILES: C[C@@H](Oc1ccc2[nH]nc(-c3cnc(N4CC(C)(NCCS(C)(=O)=O)C4)c(C#N)c3)c2c1)c1c(Cl)cncc1Cl] |
| (2S)-1-[[1-[5-[5- [(1R)-1-(3,5- dichloro-4- pyridyl)ethoxy]-4- fluoro-1H-indazol-3- yl]-3-fluoro-2- pyridyl]-3-methyl- azetidin-3- yl]amino]propan-2- ol | 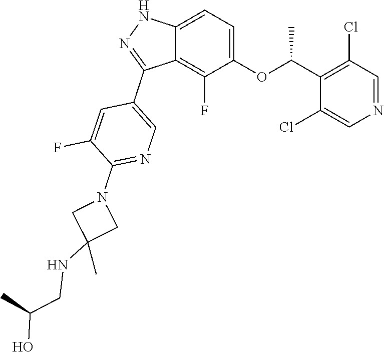 [SMILES: C[C@H](O)CNC1(C)CN(c2ncc(-c3n[nH]c4ccc(O[C@H](C)c5c(Cl)cncc5Cl)c(F)c34)cc2F)C1] |
| (2R)-1-[[1-[5-[5- [(1R)-1-(3,5- dichloro-4- pyridyl)ethoxy]-4- fluoro-1H-indazol-3- yl]-3-fluoro-2- pyridyl]-3-methyl- azetidin-3- yl]amino]propan-2- ol | 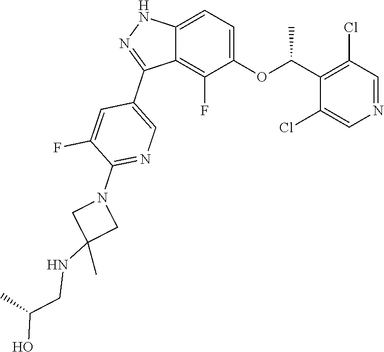 [SMILES: C[C@@H](O)CNC1(C)CN(c2ncc(-c3n[nH]c4ccc(O[C@H](C)c5c(Cl)cncc5Cl)c(F)c34)cc2F)C1] |
| 1-[[1-[5-[5-[(1R)-1- (3,5-dichloro-4- pyridyl)ethoxy]-4- fluoro-1H-indazol-3- yl]-3-fluoro-2- pyridyl]-3-methyl- azetidin-3- yl]amino]-2-methyl- propan-2-ol | 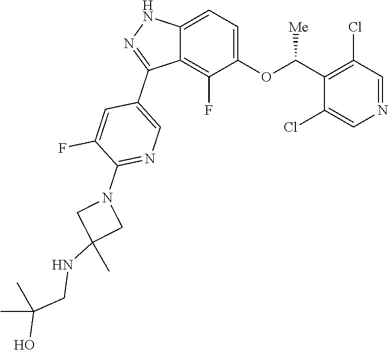 [SMILES: CC(C)(O)CNC1(C)CN(c2ncc(-c3n[nH]c4ccc(OCc5c(Cl)cncc5Cl)c(F)c34)cc2F)C1 ; "Me"] |
| 4-[[1-[5-[5-[(1R)-1- (3,5-dichloro-4- pyridyl)ethoxy]-6- fluoro-1H-indazol-3- yl]-2-pyridyl]-3- methyl-azetidin-3- yl]methyl]morpholine | 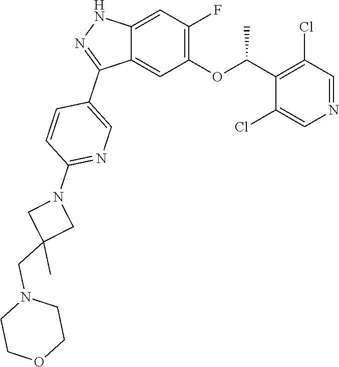 [SMILES: C[C@@H](Oc1cc2c(-c3ccc(N4CC(C)(CN5CCOCC5)C4)nc3)n[nH]c2cc1F)c1c(Cl)cncc1Cl] |
| 5-[(1R)-1-(3,5- dichloro-4- pyridyl)ethoxy]-6- fluoro-3-[6-[3- methyl-3- (pyrrolidin-1- ylmethyl)azetidin-1- yl]-3-pyridyl]-1H- indazole | 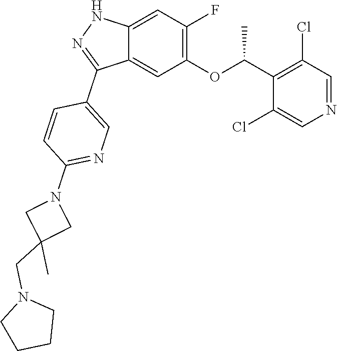 [SMILES: C[C@@H](Oc1cc2c(-c3ccc(N4CC(C)(CN5CCCC5)C4)nc3)n[nH]c2cc1F)c1c(Cl)cncc1Cl] |
| N-[[1-[5-[5-[(1R)-1- (3,5-dichloro-4- pyridyl)ethoxy]-6- fluoro-1H-indazol-3- yl]-2-pyridyl]-3- methyl-azetidin-3- yl]methyl]propan-2- amine | 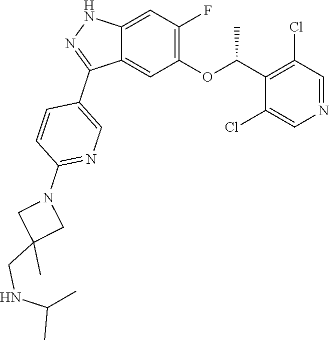 [SMILES: CC(C)NCC1(C)CN(c2ccc(-c3n[nH]c4cc(F)c(O[C@H](C)c5c(Cl)cncc5Cl)cc34)cn2)C1] |
| N-[[1-[5-[5-[(1R)-1- (3,5-dichloro-4- pyridyl)ethoxy]-4- fluoro-1H-indazol-3- yl]-2-pyridyl]-3- methyl-azetidin-3- yl]methyl]propan-2- amine | 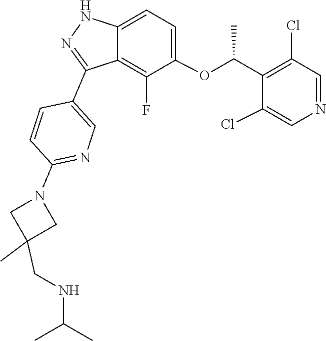 [SMILES: CC(C)NCC1(C)CN(c2ccc(-c3n[nH]c4ccc(O[C@H](C)c5c(Cl)cncc5Cl)c(F)c34)cn2)C1] |
| 5-[(1R)-1-(3,5- dichloro-4- pyridyl)ethoxy]-4- fluoro-3-[6-[3- methyl-3- (pyrrolidin-1- ylmethyl)azetidin-1- yl]-3-pyridyl]-1H- indazole | 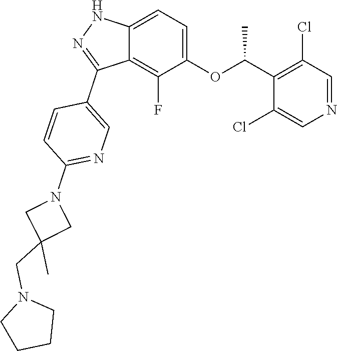 [SMILES: C[C@@H](Oc1ccc2[nH]nc(-c3ccc(N4CC(C)(CN5CCCC5)C4)nc3)c2c1F)c1c(Cl)cncc1Cl] |
| 5-[(1R)-1-(3,5- dichloro-4- pyridyl)ethoxy]-3- [6-[3- (dimethylphosphoryl methyl)-3-methyl- azetidin-1-yl]-3- pyridyl]-4-fluoro- 1H-indazole | 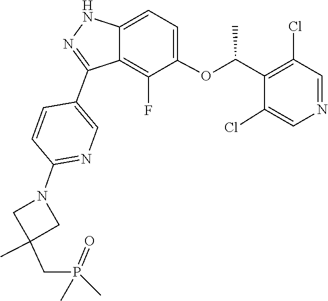 [SMILES: C[C@@H](Oc1ccc2[nH]nc(-c3ccc(N4CC(C)(CP(C)(C)=O)C4)nc3)c2c1F)c1c(Cl)cncc1Cl] |
| 1-[5-[5-[(1R)-1-(3,5- dichloro-4- pyridyl)ethoxy]-4- fluoro-1H-indazol-3- yl]-2-pyridyl]-N- isopropyl-3-methyl- azetidin-3-amine | 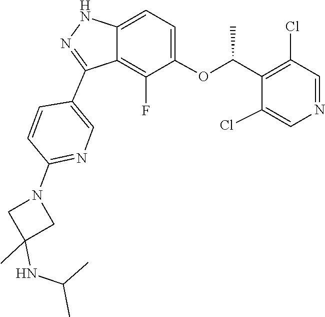 [SMILES: CC(C)NC1(C)CN(c2ccc(-c3n[nH]c4ccc(O[C@H](C)c5c(Cl)cncc5Cl)c(F)c34)cn2)C1] |
| 5-[(1R)-1-(3,5- dichloro-4- pyridyl)ethoxy]-4- fluoro-3-[6-(3- methyl-3-pyrrolidin- 1-yl-azetidin-1-yl)- 3-pyridyl]-1H- indazole | 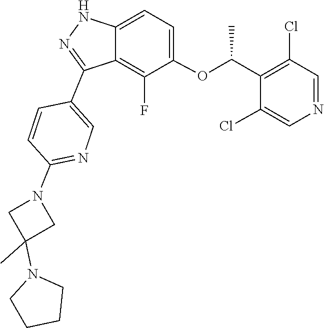 [SMILES: C[C@@H](Oc1ccc2[nH]nc(-c3ccc(N4CC(C)(N5CCCC5)C4)nc3)c2c1F)c1c(Cl)cncc1Cl] |
| 2-[[1-[5-[5-[(1R)-1- (3,5-dichloro-4- pyridyl)ethoxy]-4- fluoro-1H-indazol-3- yl]-2-pyridyl]-3- methyl-azetidin-3- yl]amino]ethanol | 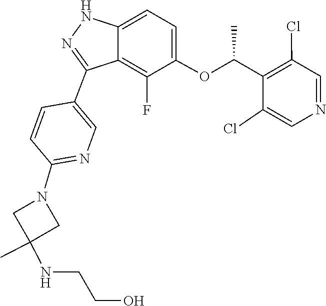 [SMILES: C[C@@H](Oc1ccc2[nH]nc(-c3ccc(N4CC(C)(NCCO)C4)nc3)c2c1F)c1c(Cl)cncc1Cl] |
| 5-[5-[(1R)-1-(3,5- dichloro-4- pyridyl)ethoxy]-4- fluoro-1H-indazol-3- yl]-2-[3- [(isopropylamino)- methyl]-3-methyl- azetidin-1- yl]pyridine-3- carbonitrile | 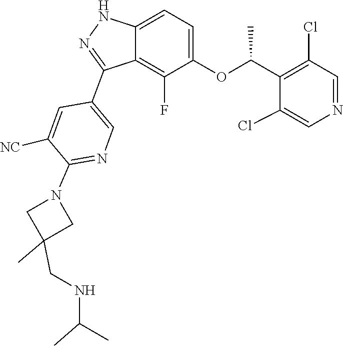 [SMILES: CC(C)NCC1(C)CN(c2ncc(-c3n[nH]c4ccc(O[C@H](C)c5c(Cl)cncc5Cl)c(F)c34)cc2C#N)C1] |
| 2-[[1-[5-[5-[(1R)-1- (3,5-dichloro-4- pyridyl)ethoxy]-1H- indazol-3-yl]-3- fluoro-2-pyridyl]-3- methyl-azetidin-3- yl]methylamino]ethanol | 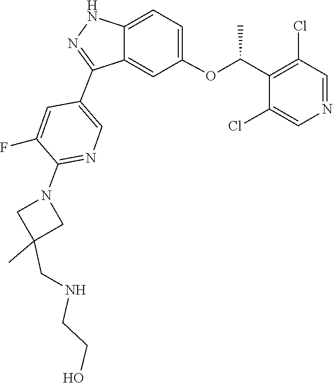 [SMILES: C[C@@H](Oc1ccc2[nH]nc(-c3cnc(N4CC(C)(CNCCO)C4)c(F)c3)c2c1)c1c(Cl)cncc1Cl] |
| 5-[(1R)-1-(3,5- dichloro-4- pyridyl)ethoxy]-3- [6-[3- (dimethylphosphoryl methyl)-3-methyl- azetidin-1-yl]-5- fluoro-3-pyridyl]-6- fluoro-1H-indazole | 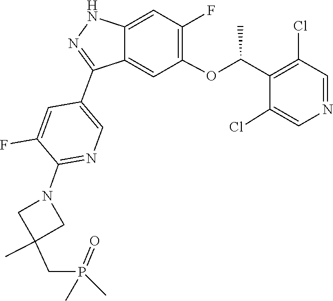 [SMILES: C[C@@H](Oc1cc2c(-c3cnc(N4CC(C)(CP(C)(C)=O)C4)c(F)c3)n[nH]c2cc1F)c1c(Cl)cncc1Cl] |
| 5-[5-[(1R)-1-(3,5- dichloro-4- pyridyl)ethoxy]-1H- indazol-3-yl]-2-[3- [(isopropylamino) methyl]-3-methyl- azetidin-1- yl]pyridine-3- carbonitrile | 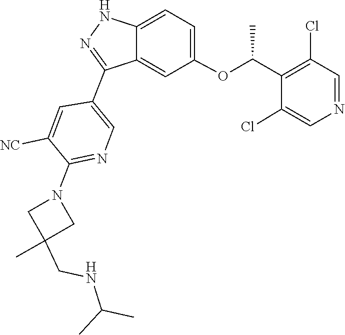 [SMILES: CC(C)NCC1(C)CN(c2ncc(-c3n[nH]c4ccc(O[C@H](C)c5c(Cl)cncc5Cl)cc34)cc2C#N)C1] |
| 1-[3-cyano-5-[5- [(1R)-1-(3,5- dichloro-4- pyridyl)ethoxy]-1H- indazol-3-yl]-2- pyridyl]-N-(2- hydroxyethyl)-3- methyl-azetidine-3- carboxamide | 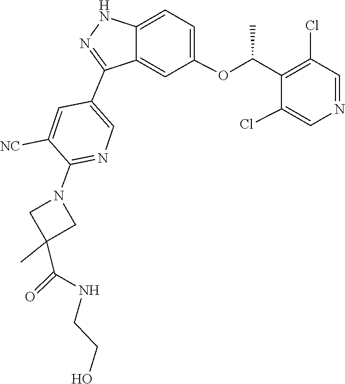 [SMILES: C[C@@H](Oc1ccc2[nH]nc(-c3cnc(N4CC(C)(C(=O)NCCO)C4)c(C#N)c3)c2c1)c1c(Cl)cncc1Cl] |
| 1-[5-[5-[(1R)-1-(3,5- dichloro-4- pyridyl)ethoxy]-1H- indazol-3-yl]-3- fluoro-2-pyridyl]-N- (2-hydroxyethyl)-3- methyl-azetidine-3- carboxamide | 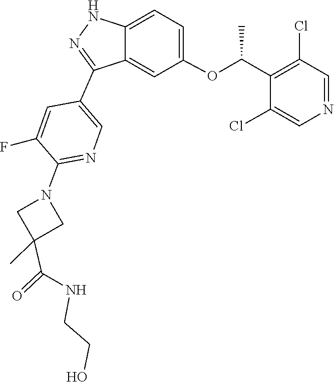 [SMILES: C[C@@H](Oc1ccc2[nH]nc(-c3cnc(N4CC(C)(C(=O)NCCO)C4)c(F)c3)c2c1)c1c(Cl)cncc1Cl] |
| 1-[5-[5-[(1R)-1-(3,5- dichloro-4- pyridyl)ethoxy]-1H- indazol-3-yl]-3- fluoro-2-pyridyl]-N- (dimethylphosphoryl methyl)-3-methyl- azetidin-3-amine | 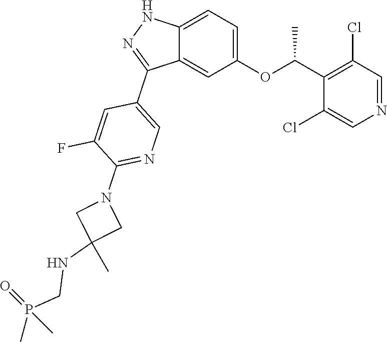 [SMILES: C[C@@H](Oc1ccc2[nH]nc(-c3cnc(N4CC(C)(NCP(C)(C)=O)C4)c(F)c3)c2c1)c1c(Cl)cncc1Cl] |
| 5-[5-[(1R)-1-(3,5- dichloro-4- pyridyl)ethoxy]-1H- indazol-3-yl]-2-[3- methyl-3- (phosphorosomethyl- amino)azetidin-1- yl]pyridine-3- carbonitrile | 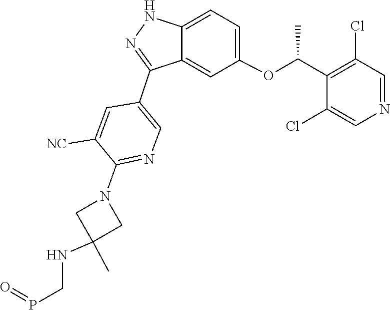 |
| 1-[5-[5-[(1R)-1-(3,5- dichloro-4- pyridyl)ethoxy]-6- methoxy-1H- indazol-3-yl]-3- fluoro-2-pyridyl]-N- (dimethylphosphoryl methyl)-3-methyl- azetidin-3-amine | 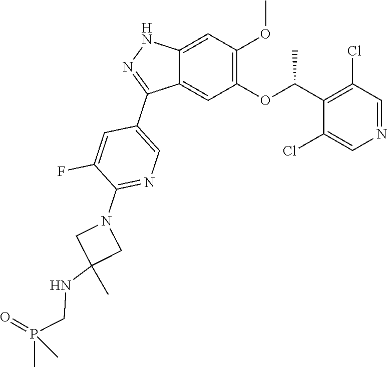 [SMILES: COc1cc2[nH]nc(-c3cnc(N4CC(C)(NCP(C)(C)=O)C4)c(F)c3)c2cc1O[C@H](C)c1c(Cl)cncc1Cl] |
| 5-[5-[(1R)-1-(3,5- dichloro-4- pyridyl)ethoxy]-6- methoxy-1H- indazol-3-yl]-2-[3- (dimethylphosphoryl- methylamino)-3- methyl-azetidin-1- yl]pyridine-3- carbonitrile | 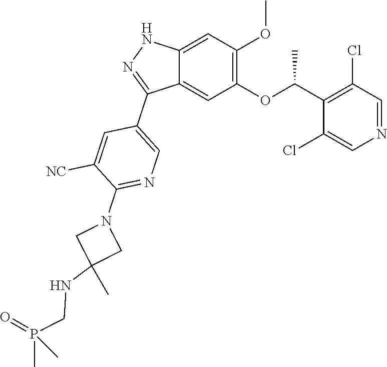 [SMILES: COc1cc2[nH]nc(-c3cnc(N4CC(C)(NCP(C)(C)=O)C4)c(C#N)c3)c2cc1O[C@H](C)c1c(Cl)cncc1Cl] |
| 1-[5-[5-[(1R)-1-(3,5- dichloro-4- pyridyl)ethoxy]-6- fluoro-1H-indazol-3- yl]-3-fluoro-2- pyridyl]-N- (dimethyl- phosphorylmethyl)- 3-methyl-azetidin-3- amine | 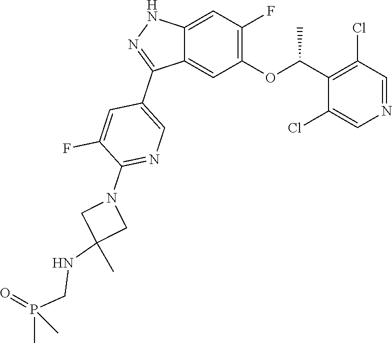 [SMILES: C[C@@H](Oc1cc2c(-c3cnc(N4CC(C)(NCP(C)(C)=O)C4)c(F)c3)n[nH]c2cc1F)c1c(Cl)cncc1Cl] |

In other embodiments, the compounds of the disclosure are the compounds shown in the following table, or pharmaceutically acceptable salts thereof:

| | |
|---|---|
| 2-[3-amino-3- (hydroxymethyl)azetidin- 1-yl]-5-[5-[(1R)-1-(3,5- dichloro-4- pyridyl)ethoxy]-6- methoxy-1H-indazol-3- yl]pyridine-3-carbonitrile | 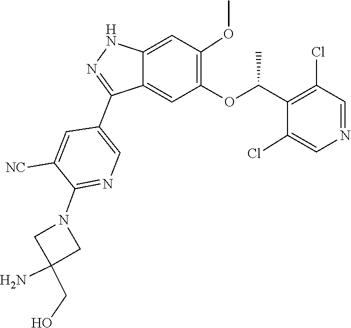 [SMILES: COc1cc2[nH]nc(-c3cnc(N4CC(N)(CO)C4)c(C#N)c3)c2cc1O[C@H](C)c1c(Cl)cncc1Cl] |
| 2-[3-amino-3- (isopropoxymethyl)azetidin- 1-yl]-5-[5-[(1R)-1-(3,5- dichloro-4-pyridyl)ethoxy]-6- methoxy-1H-indazol-3- yl]pyridine-3-carbonitrile | 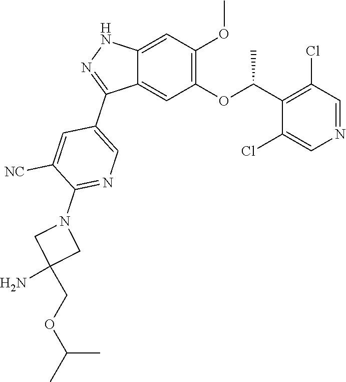 [SMILES: COc1cc2[nH]nc(-c3cnc(N4CC(N)(COC(C)C)C4)c(C#N)c3)c2cc1O[C@H](C)c1c(Cl)cncc1Cl] |
| 2-[3-amino-3- (ethoxymethyl)azetidin-1- yl]-5-[5-[(1R)-1-(3,5- dichloro-4- pyridyl)ethoxy]-6- methoxy-1H-indazol-3- yl]pyridine-3-carbonitrile | 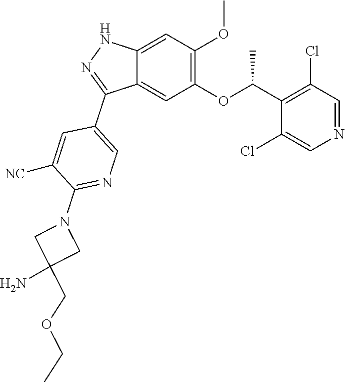 [SMILES: CCOCC1(N)CN(c2ncc(-c3n[nH]c4cc(OC)c(O[C@H](C)c5c(Cl)cncc5Cl)cc34)cc2C#N)C1] |
| 1-[6-[5-[(1R)-1-(3,5-dichloro- 4-pyridyl)ethoxy]-1H- indazol-3-yl]pyridazin-3-yl]- 3-(5,6-dihydro-4H- pyrrolo[1,2-b]pyrazol-3- ylmethyl)azetidin-3-amine | 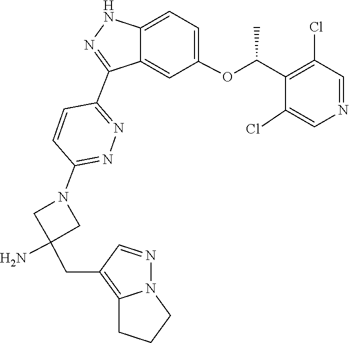 [SMILES: C[C@@H](Oc1ccc2[nH]nc(-c3ccc(N4CC(N)(Cc5cnn6c5CCC6)C4)nn3)c2c1)c1c(Cl)cncc1Cl] |
| (3S)-1-[6-[5-[(1R)-1-(3,5- dichloro-4- pyridyl)ethoxy]-1H- indazol-3-yl]pyridazin-3- yl]-3-(2- methylsulfonylethyl)- pyrrolidin-3-amine | 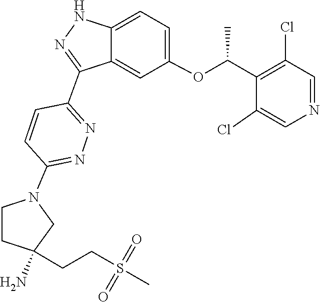 [SMILES: C[C@@H](Oc1ccc2[nH]nc(-c3ccc(N4CC[C@](N)(CCS(C)(=O)=O)C4)nn3)c2c1)c1c(Cl)cncc1Cl] |
| (3R)-1-[6-[5-[(1R)-1-(3,5- dichloro-4-pyridyl)ethoxy]- 1H-indazol-3-yl]pyridazin-3- yl]-3-(2- methylsulfonylethyl) pyrrolidin-3-amine | 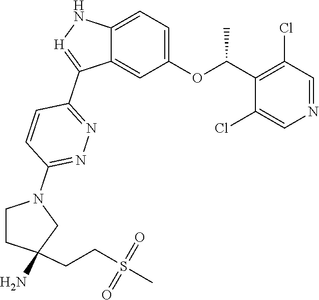 |
| 1-[6-[5-[(1R)-1-(3,5- dichloro-4- pyridyl)ethoxy]-1H- indazol-3-yl]pyridazin-3- yl]-3-(thiazol-2- ylmethyl)azetidin-3-amine | 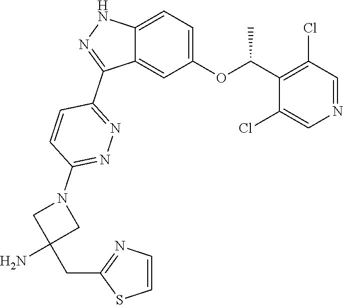 [SMILES: C[C@@H](Oc1ccc2[nH]nc(-c3ccc(N4CC(N)(Cc5nccs5)C4)nn3)c2c1)c1c(Cl)cncc1Cl] |
| 1-[6-[5-[(1R)-1-(3,5-dichloro- 4-pyridyl)ethoxy]-1H- indazol-3-yl]pyridazin-3-yl]- 3-(isothiazol-3- ylmethyl)azetidin-3-amine | 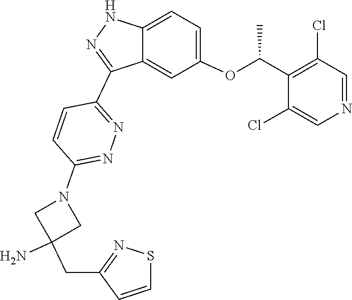 [SMILES: C[C@@H](Oc1ccc2[nH]nc(-c3ccc(N4CC(N)(Cc5ccsn5)C4)nn3)c2c1)c1c(Cl)cncc1Cl] |
| 1-[6-[5-[(1R)-1-(3,5- dichloro-4- pyridyl)ethoxy]-1H- indazol-3-yl]pyridazin-3- yl]-3-(2- pyridylmethyl)azetidin-3- amine | 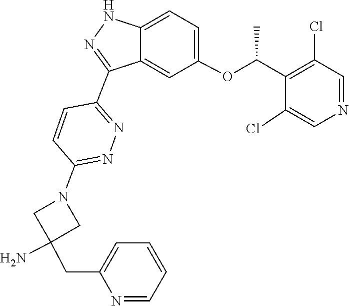 [SMILES: C[C@@H](Oc1ccc2[nH]nc(-c3ccc(N4CC(N)(Cc5ccccn5)C4)nn3)c2c1)c1c(Cl)cncc1Cl] |
| 1-[5-[5-[(1R)-1-(3,5-dichloro- 4-pyridyl)ethoxy]-1H- indazol-3-yl]-3-fluoro-2- pyridyl]-3-(thiazol-2- ylmethyl)azetidin-3-amine | 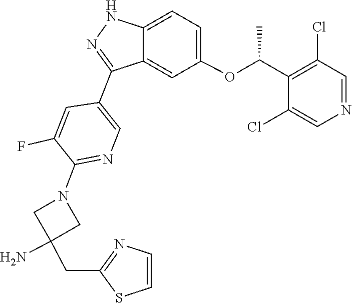 [SMILES: C[C@@H](Oc1ccc2[nH]nc(-c3cnc(N4CC(N)(Cc5nccs5)C4)c(F)c3)c2c1)c1c(Cl)cncc1Cl] |
| 1-[6-[5-[(1R)-1-(3,5- dichloro-4- pyridyl)ethoxy]-1H- indazol-3-yl]pyridazin-3- yl]-3-(pyrimidin-2- ylmethyl)azetidin-3-amine | 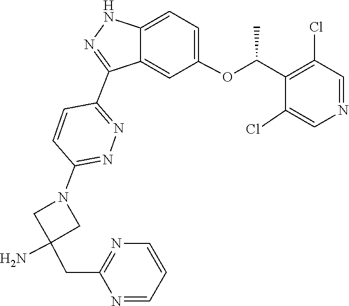 [SMILES: C[C@@H](Oc1ccc2[nH]nc(-c3ccc(N4CC(N)(Cc5ncccn5)C4)nn3)c2c1)c1c(Cl)cncc1Cl] |
| 1-[5-[5-[(1R)-1-(3,5-dichloro- 4-pyridyl)ethoxy]-1H- indazol-3-yl]-3-fluoro-2- pyridyl]-3-[(3- methylisothiazol-4- yl)methyl]azetidin-3-amine | 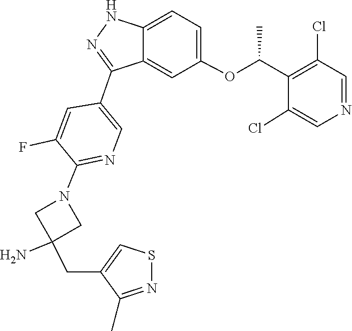 [SMILES: Cc1nscc1CC1(N)CN(c2ncc(-c3n[nH]c4ccc(O[C@H](C)c5c(Cl)cncc5Cl)cc34)cc2F)C1] |
| 1-[6-[5-[(1R)-1-(3,5- dichloro-4- pyridyl)ethoxy]-1H- indazol-3-yl]pyridazin-3- yl]-3-[(3-methylisothiazol- 4-yl)methyl]azetidin-3- amine | 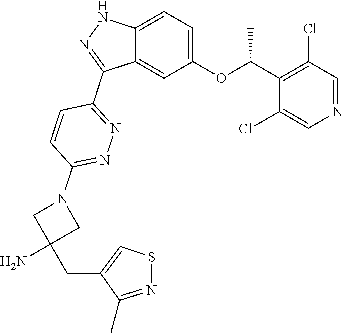 [SMILES: Cc1nscc1CC1(N)CN(c2ccc(-c3n[nH]c4ccc(O[C@H](C)c5c(Cl)cncc5Cl)cc34)nn2)C1] |
| 1-[5-[5-[(1R)-1-(3,5-dichloro- 4-pyridyl)ethoxy]-1H- indazol-3-yl]-3-fluoro-2- pyridyl]-3-(isothiazol-3- ylmethyl)azetidin-3-amine | 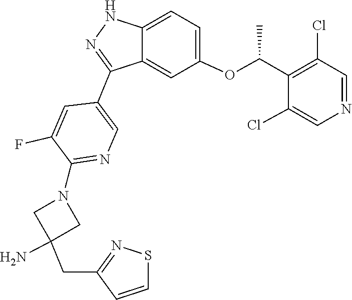 [SMILES: C[C@@H](Oc1ccc2[nH]nc(-c3cnc(N4CC(N)(Cc5ccsn5)C4)c(F)c3)c2c1)c1c(Cl)cncc1Cl] |
| 2-[3-amino-1-[6-[5-[(1R)- 1-(3,5-dichloro-4- pyridyl)ethoxy]-1H- indazol-3-yl]pyridazin-3- yl]azetidin-3-yl]-1- pyrrolidin-1-yl-ethanone | 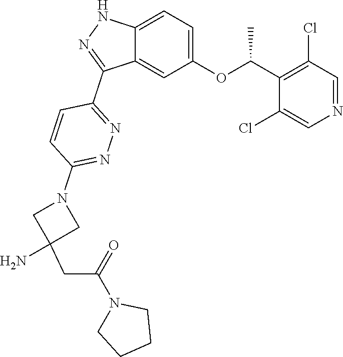 [SMILES: C[C@@H](Oc1ccc2[nH]nc(-c3ccc(N4CC(N)(CC(=O)N5CCCC5)C4)nn3)c2c1)c1c(Cl)cncc1Cl] |
| 1-[6-[5-[(1R)-1-(3,5-dichloro- 4-pyridyl)ethoxy]-1H- indazol-3-yl]pyridazin-3-yl]- 3-[(1,5-dimethylpyrazol-4- yl)methyl]azetidin-3-amine | 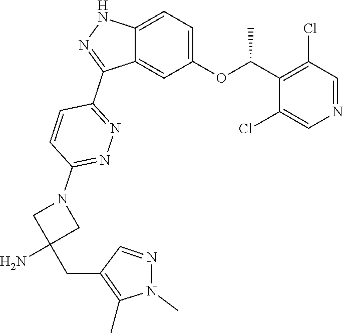 [SMILES: Cc1c(CC2(N)CN(c3ccc(-c4n[nH]c5ccc(O[C@H](C)c6c(Cl)cncc6Cl)cc45)nn3)C2)cnn1C] |
| 1-[6-[5-[(1R)-1-(3,5- dichloro-4- pyridyl)ethoxy]-1H- indazol-3-yl]pyridazin-3- yl]-3-[(1,3- dimethylpyrazol-4- yl)methyl]azetidin-3-amine | 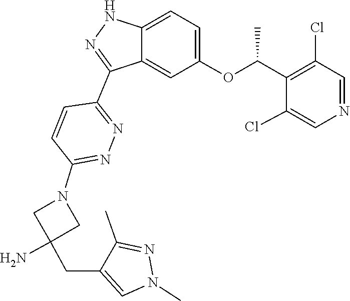 [SMILES: Cc1nn(C)cc1CC1(N)CN(c2ccc(-c3n[nH]c4ccc(O[C@H](C)c5c(Cl)cncc5Cl)cc34)nn2)C1] |
| 2-[3-amino-3-[(3- methylisothiazol-4- yl)methyl]azetidin-1-yl]-5-[5- [(1R)-1-(3,5-dichloro-4- pyridyl)ethoxy]-6-methoxy- 1H-indazol-3-yl]pyridine-3- carbonitrile | 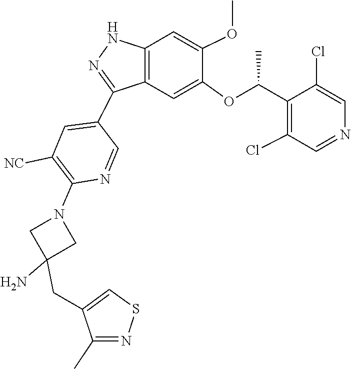 [SMILES: COc1cc2[nH]nc(-c3cnc(N4CC(N)(Cc5csnc5C)C4)c(C#N)c3)c2cc1O[C@H](C)c1c(Cl)cncc1Cl] |
| 1-[6-[5-[(1R)-1-(3,5- dichloro-4- pyridyl)ethoxy]-1H- indazol-3-yl]pyridazin-3- yl]-3-[(4-methylthiazol-2- yl)methyl]azetidin-3-amine | 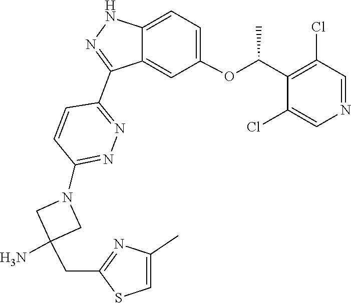 |
| 1-[6-[5-[(1R)-1-(3,5-dichloro- 4-pyridyl)ethoxy]-1H- indazol-3-yl]pyridazin-3-yl]- 3-(4-methylthiazol-2- yl)azetidin-3-amine | 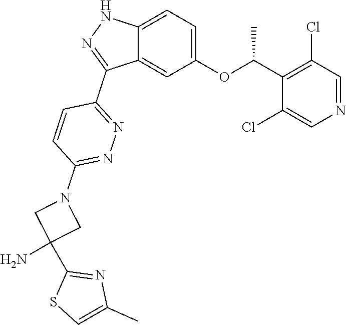 [SMILES: Cc1csc(C2(N)CN(c3ccc(-c4n[nH]c5ccc(O[C@H](C)c6c(Cl)cncc6Cl)cc45)nn3)C2)n1] |
| 2-[3-amino-3-(thiazol-2- ylmethyl)azetidin-1-yl]-5- [5-[(1R)-1-(3,5-dichloro-4- pyridyl)ethoxy]-6- methoxy-1H-indazol-3- yl]pyridine-3-carbonitrile | 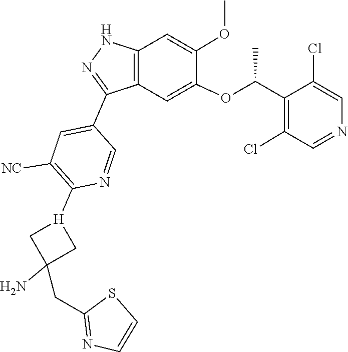 |
| 2-[3-amino-3-(5,6-dihydro- 4H-pyrrolo[1,2-b]pyrazol-3- ylmethyl)azetidin-1-yl]-5-[5- [(1R)-1-(3,5-dichloro-4- pyridyl)ethoxy]-6-methoxy- 1H-indazol-3-yl]pyridine-3- carbonitrile | 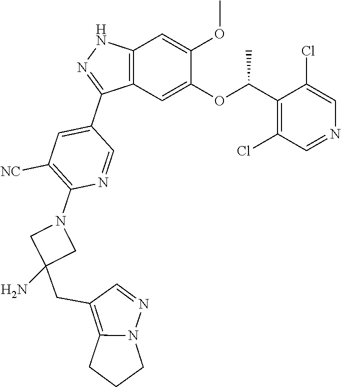 [SMILES: COc1cc2[nH]nc(-c3cnc(N4CC(N)(Cc5cnn6c5CCC6)C4)c(C#N)c3)c2cc1O[C@H](C)c1c(Cl)cncc1Cl] |
| 2-[3-amino-3-(isothiazol- 3-ylmethyl)azetidin-1-yl]- 5-[5-[(1R)-1-(3,5-dichloro- 4-pyridyl)ethoxy]-6- methoxy-1H-indazol-3- yl]pyridine-3-carbonitrile | 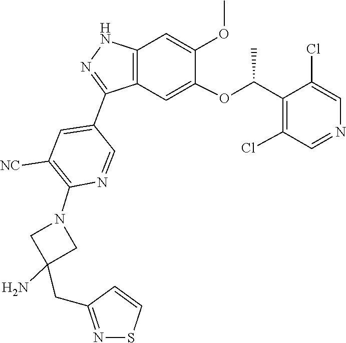 [SMILES: COc1cc2[nH]nc(-c3cnc(N4CC(N)(Cc5ccsn5)C4)c(C#N)c3)c2cc1O[C@H](C)c1c(Cl)cncc1Cl] |
| 2-[3-amino-3-(2- methylpyrazol-3-yl)azetidin- 1-yl]-5-[5-[(1R)-1-(3,5- dichloro-4-pyridyl)ethoxy]-6- methoxy-1H-indazol-3- yl]pyridine-3-carbonitrile | 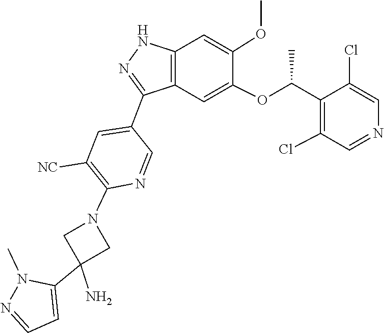 [SMILES: COc1cc2[nH]nc(-c3cnc(N4CC(N)(c5ccnn5C)C4)c(C#N)c3)c2cc1O[C@H](C)c1c(Cl)cncc1Cl] |
| 2-[3-amino-3-(4- methylthiazol-2- yl)azetidin-1-yl]-5-[5- [(1R)-1-(3,5-dichloro-4- pyridyl)ethoxy]-6- methoxy-1H-indazol-3- yl]pyridine-3-carbonitrile | 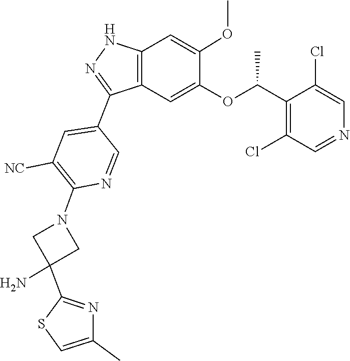 [SMILES: COc1cc2[nH]nc(-c3cnc(N4CC(N)(c5nc(C)cs5)C4)c(C#N)c3)c2cc1O[C@H](C)c1c(Cl)cncc1Cl] |
| 2-[3-amino-3-[(4- methylthiazol-2- yl)methyl]azetidin-1-yl]-5-[5- [(1R)-1-(3,5-dichloro-4- pyridyl)ethoxy]-6-methoxy- 1H-indazol-3-yl]pyridine-3- carbonitrile | 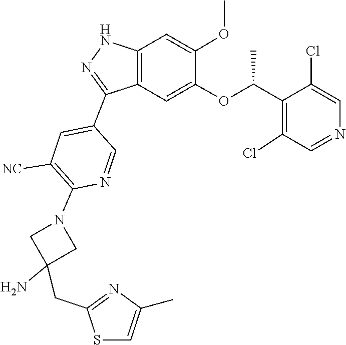 [SMILES: COc1cc2[nH]nc(-c3cnc(N4CC(N)(Cc5nc(C)cs5)C4)c(C#N)c3)c2cc1O[C@H](C)c1c(Cl)cncc1Cl] |
| 1-[6-[5-[(1R)-1-(3,5- dichloro-4- pyridyl)ethoxy]-1H- indazol-3-yl\|pyridazin-3- yl]-3-(6- methoxypyridazin-3- yl)azetidin-3-amine | 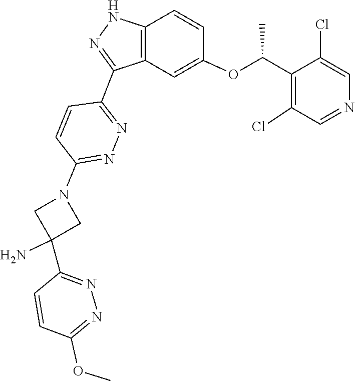 [SMILES: COc1ccc(C2(N)CN(c3ccc(-c4n[nH]c5ccc(O[C@H](C)c6c(Cl)cncc6Cl)cc45)nn3)C2)nn1] |
| 2-[3-amino-3-(6- methoxypyridazin-3- yl)azetidin-1-yl]-5-[5-[(1R)- 1-(3,5-dichloro-4- pyridyl)ethoxy]-6-methoxy- 1H-indazol-3-yl]pyridine-3- carbonitrile | 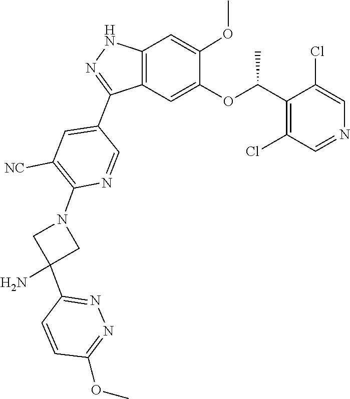 [SMILES: COc1ccc(C2(N)CN(c3ncc(-c4n[nH]c5cc(OC)c(O[C@H](C)c6c(Cl)cncc6Cl)cc45)cc3C#N)C2)nn1] |
| 3-[3-amino-1-[6-[5-[(1R)- 1-(3,5-dichloro-4- pyridyl)ethoxy]-1H- indazol-3-yl]pyridazin-3- yl]azetidin-3-yl]-1H- pyridazin-6-one | 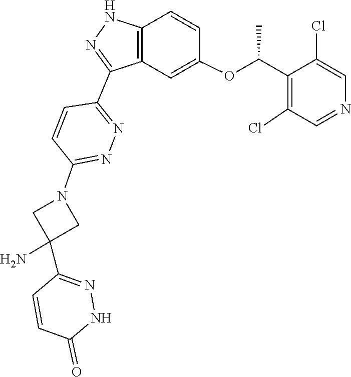 [SMILES: C[C@@H](Oc1ccc2[nH]nc(-c3ccc(N4CC(N)(c5ccc(=O)[nH]n5)C4)nn3)c2c1)c1c(Cl)cncc1Cl] |
| 2-[3-amino-3-(6-oxo-1H- pyridazin-3-yl)azetidin-1-yl]- 5-[5-[(1R)-1-(3,5-dichloro-4- pyridyl)ethoxy]-6-methoxy- 1H-indazol-3-yl]pyridine-3- carbonitrile | 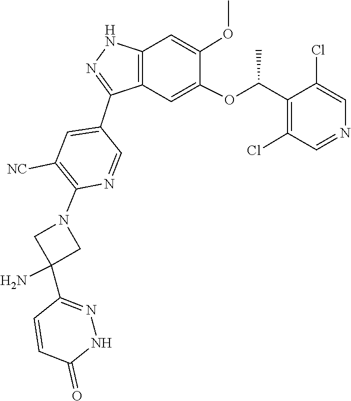 [SMILES: COc1cc2[nH]nc(-c3cnc(N4CC(N)(c5ccc(=O)[nH]n5)C4)c(C#N)c3)c2cc1O[C@H](C)c1c(Cl)cncc1Cl] |
| 1-[6-[5-[(1R)-1-(3,5- dichloro-4- pyridyl)ethoxy]-1H- indazol-3-yl]pyridazin-3- yl]-3-(2,2,2- trifluoroethyl)azetidin-3- amine | 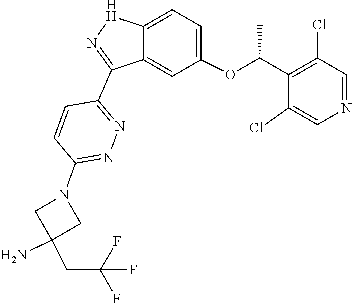 |
| 1-[6-[5-[(1R)-1-(3,5-dichloro- 4-pyridyl)ethoxy]-1H- indazol-3-yl]pyridazin-3-yl]- 3-[(1-ethylpyrazol-4- yl)methyl]azetidin-3-amine | 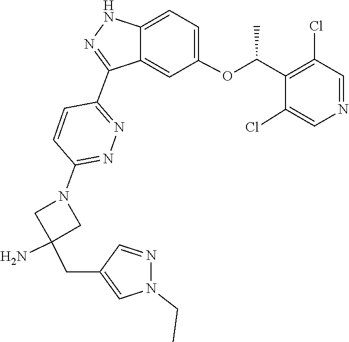 [SMILES: CCn1cc(CC2(N)CN(c3ccc(-c4n[nH]c5ccc(O[C@H](C)c6c(Cl)cncc6Cl)cc45)nn3)C2)cn1] |
| 1-[6-[5-[(1R)-1-(3,5- dichloro-4- pyridyl)ethoxy]-1H- indazol-3-yl]pyridazin-3- yl]-3-[(1-isopropylpyrazol- 4-yl)methyl]azetidin-3- amine | 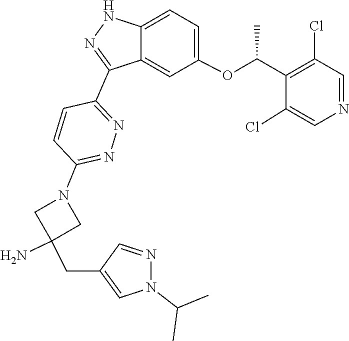 [SMILES: CC(C)n1cc(CC2(N)CN(c3ccc(-c4n[nH]c5ccc(O[C@H](C)c6c(Cl)cncc6Cl)cc45)nn3)C2)cn1] |
| 3-[(1-cyclopropylpyrazol-4- yl)methyl]-1-[6-[5-[(1R)-1- (3,5-dichloro-4- pyridyl)ethoxy]-1H-indazol- 3-yl]pyridazin-3-yl]azetidin- 3-amine | 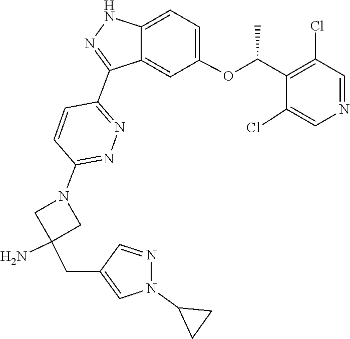 [SMILES: C[C@@H](Oc1ccc2[nH]nc(-c3ccc(N4CC(N)(Cc5cnn(C6CC6)c5)C4)nn3)c2c1)c1c(Cl)cncc1Cl] |
| 1-[4-[[3-amino-1-[6-[5- [(1R)-1-(3,5-dichloro-4- pyridyl)ethoxy]-1H- indazol-3-yl]pyridazin-3- yl]azetidin-3- yl]methyl]pyrazol-1-yl]-2- methyl-propan-2-ol | 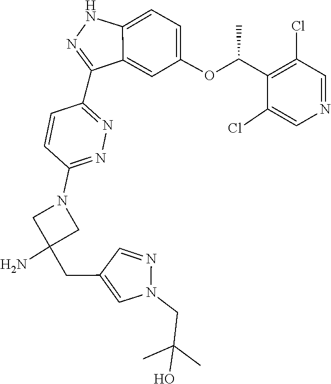 [SMILES: C[C@@H](Oc1ccc2[nH]nc(-c3ccc(N4CC(N)(Cc5cnn(CC(C)(C)O)c5)C4)nn3)c2c1)c1c(Cl)cncc1Cl] |
| 1-[6-[5-[(1R)-1-(3,5-dichloro- 4-pyridyl)ethoxy]-1H- indazol-3-yl]pyridazin-3-yl]- 3-[[1- (trifluoromethyl)pyrazol-4- yl]methyl]azetidin-3-amine | 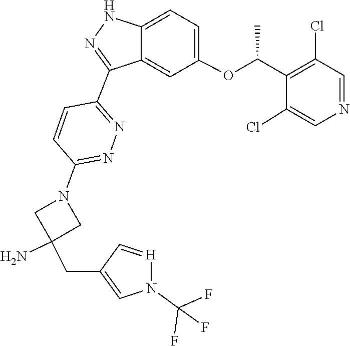 |
| 1-[6-[5-[(1R)-1-(3,5- dichloro-4- pyridyl)ethoxy]-1H- indazol-3-yl]pyridazin-3- yl]-3-[[1- (difluoromethyl)pyrazol-4- yl]methyl]azetidin-3-amine | 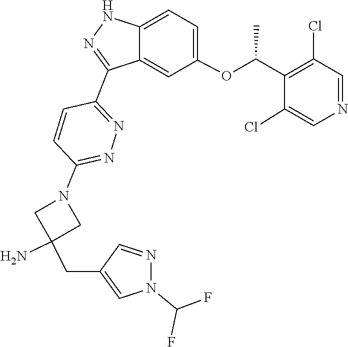 [SMILES: C[C@@H](Oc1ccc2[nH]nc(-c3ccc(N4CC(N)(Cc5cnn(C(F)F)c5)C4)nn3)c2c1)c1c(Cl)cncc1Cl] |
| 2-[4-[[3-amino-1-[6-[5-[(1R)- 1-(3,5-dichloro-4- pyridyl)ethoxy]-1H-indazol- 3-yl\|pyridazin-3-yl]azetidin- 3-yl]methyl]pyrazol-1- yl]ethanol | 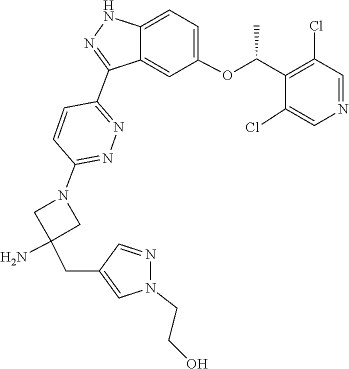 [SMILES: C[C@@H](Oc1ccc2[nH]nc(-c3ccc(N4CC(N)(Cc5cnn(CCO)c5)C4)nn3)c2c1)c1c(Cl)cncc1Cl] |
| [3-amino-1-[6-[5-[(1R)-1- (3,5-dichloro-4- pyridyl)ethoxy]-1H- indazol-3-yl]pyridazin-3- yl]azetidin-3-yl]- pyrrolidin-1-yl-methanone | 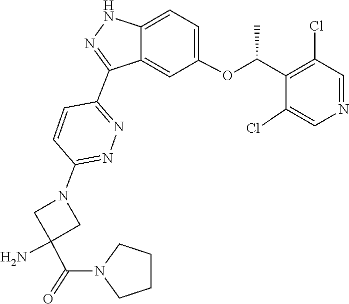 [SMILES: C[C@@H](Oc1ccc2[nH]nc(-c3ccc(N4CC(N)(C(=O)N5CCCC5)C4)nn3)c2c1)c1c(Cl)cncc1Cl] |
| [3-amino-1-[6-[5-[(1R)-1- (3,5-dichloro-4- pyridyl)ethoxy]-1H-indazol- 3-yl]pyridazin-3-yl]azetidin- 3-yl]-morpholino-methanone | 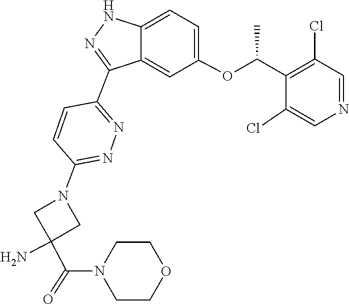 [SMILES: C[C@@H](Oc1ccc2[nH]nc(-c3ccc(N4CC(N)(C(=O)N5CCOCC5)C4)nn3)c2c1)c1c(Cl)cncc1Cl] |
| 3-amino-1-[6-[5-[(1R)-1- (3,5-dichloro-4- pyridyl)ethoxy]-1H- indazol-3-yl]pyridazin-3- yl]-N,N-diethyl-azetidine- 3-carboxamide | 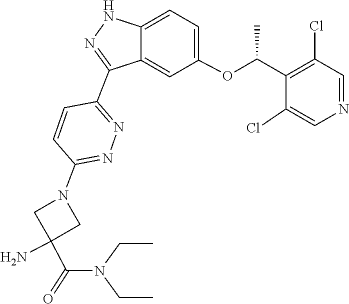 [SMILES: CCN(CC)C(=O)C1(N)CN(c2ccc(-c3n[nH]c4ccc(O[C@H](C)c5c(Cl)cncc5Cl)cc34)nn2)C1] |
| [3-amino-1-[6-[5-[(1R)-1- (3,5-dichloro-4- pyridyl)ethoxy]-1H-indazol- 3-yl]pyridazin-3-yl]azetidin- 3-yl]-[(3R)-3- hydroxypyrrolidin-1- yl]methanone | 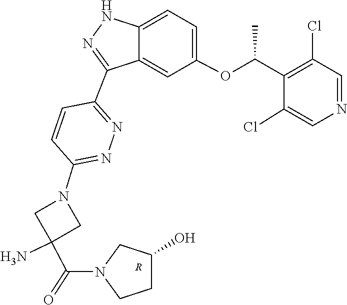 |
| [3-amino-1-[6-[5-[(1R)-1- (3,5-dichloro-4- pyridyl)ethoxy]-1H- indazol-3-yl]pyridazin-3- yl]azetidin-3-yl]-[(3S)-3- hydroxypyrrolidin-1- yl]methanone | 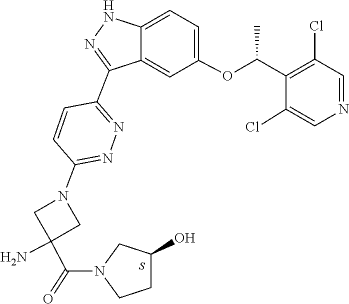 |
| [3-amino-1-[5-[5-[(1R)-1- (3,5-dichloro-4- pyridyl)ethoxy]-1H-indazol- 3-yl]-3-fluoro-2- pyridyl]azetidin-3-yl]- pyrrolidin-1-yl-methanone | 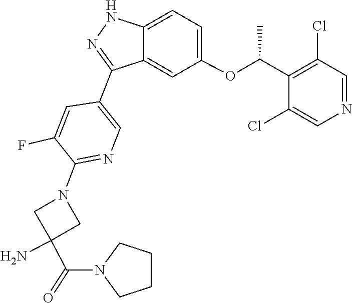 [SMILES: C[C@@H](Oc1ccc2[nH]nc(-c3cnc(N4CC(N)(C(=O)N5CCCC5)C4)c(F)c3)c2c1)c1c(Cl)cncc1Cl] |
| [3-amino-1-[5-[5-[(1R)-1- (3,5-dichloro-4- pyridyl)ethoxy]-1H- indazol-3-yl]-3-fluoro-2- pyridyl]azetidin-3-yl]- morpholino-methanone | 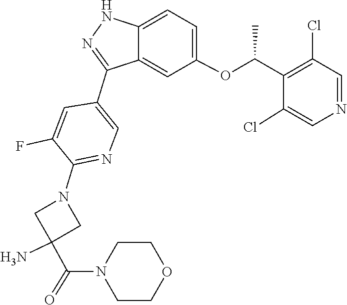 |
| 2-[3-amino-3-(pyrrolidine-1- carbonyl)azetidin-1-yl]-5-[5- [(1R)-1-(3,5-dichloro-4- pyridyl)ethoxy]-6-methoxy- 1H-indazol-3-yl]pyridine-3- carbonitrile | 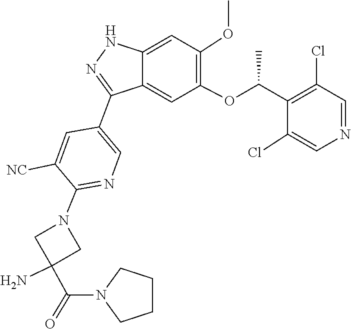 [SMILES: COc1cc2[nH]nc(-c3cnc(N4CC(N)(C(=O)N5CCCC5)C4)c(C#N)c3)c2cc1O[C@H](C)c1c(Cl)cncc1Cl] |
| 2-[3-amino-3-(morpholine- 4-carbonyl)azetidin-1-yl]- 5-[5-[(1R)-1-(3,5-dichloro- 4-pyridyl)ethoxy]-6- methoxy-1H-indazol-3- yl]pyridine-3-carbonitrile | 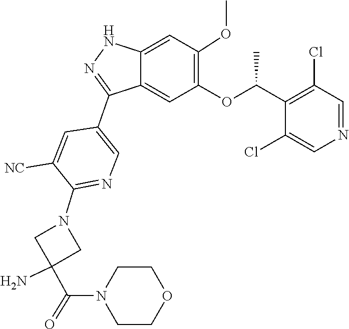 [SMILES: COc1cc2[nH]nc(-c3cnc(N4CC(N)(C(=O)N5CCOCC5)C4)c(C#N)c3)c2cc1O[C@H](C)c1c(Cl)cncc1Cl] |
| 3-amino-1-[3-cyano-5-[5- [(1R)-1-(3,5-dichloro-4- pyridyl)ethoxy]-6-methoxy- 1H-indazol-3-yl]-2-pyridyl]- N,N-dimethyl-azetidine-3- carboxamide | 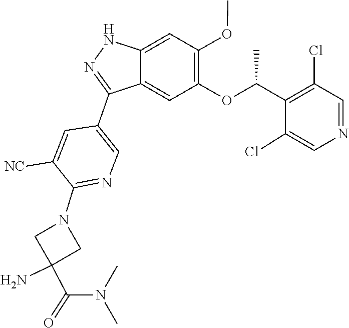 [SMILES: COc1cc2[nH]nc(-c3cnc(N4CC(N)(C(=O)N(C)C)C4)c(C#N)c3)c2cc1O[C@H](C)c1c(Cl)cncc1Cl] |
| 3-amino-1-[3-cyano-5-[5- [(1R)-1-(3,5-dichloro-4- pyridyl)ethoxy]-6- methoxy-1H-indazol-3-yl]- 2-pyridyl]-N-methyl- azetidine-3-carboxamide | 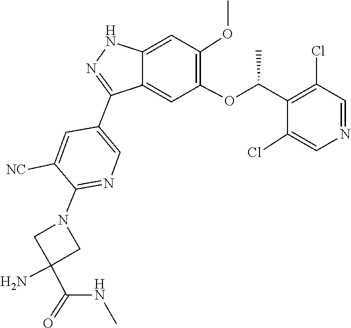 |
| 3-amino-1-[3-cyano-5-[5- [(1R)-1-(3,5-dichloro-4- pyridyl)ethoxy]-6-methoxy- 1H-indazol-3-yl]-2- pyridyl]azetidine-3- carboxamide | 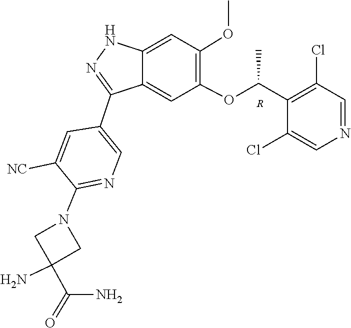 |
| 3-amino-1-[6-[5-[(1R)-1- (3,5-dichloro-4- pyridyl)ethoxy]-1H- indazol-3-yl]pyridazin-3- yl]azetidine-3- carboxamide | 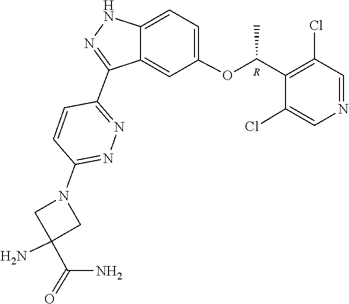 |
| [3-amino-1-[5-[5-[(1R)-1- (3,5-dichloro-4- pyridyl)ethoxy]-1H-indazol- 3-yl]-3-fluoro-2- pyridyl]azetidin-3- yl]methanol | 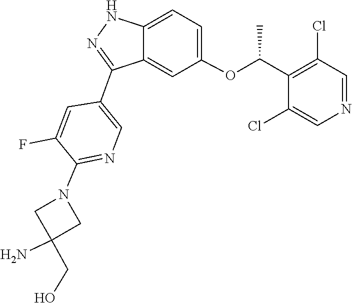 [SMILES: C[C@@H](Oc1ccc2[nH]nc(-c3cnc(N4CC(N)(CO)C4)c(F)c3)c2c1)c1c(Cl)cncc1Cl] |
| [3-amino-1-[6-[5-[(1R)-1- (3,5-dichloro-4- pyridyl)ethoxy]-1H- indazol-3-yl]pyridazin-3- yl]azetidin-3-yl]methanol | 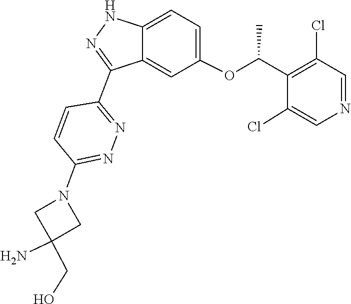 [SMILES: C[C@@H](Oc1ccc2[nH]nc(-c3ccc(N4CC(N)(CO)C4)nn3)c2c1)c1c(Cl)cncc1Cl] |

In some embodiments, formula (I) and any subgenera thereof disclosed herein, exclude by proviso any compound disclosed in International Patent Application No. PCT/US2021/065679, filed on Dec. 30, 2021.

In some embodiments, formula (I) and any subgenera thereof disclosed herein, exclude by proviso any one of the compounds shown in the following table:

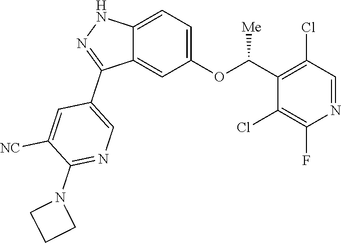 [SMILES: C[C@@H](Oc1ccc2[nH]nc(-c3cnc(N4CCC4)c(C#N)c3)c2c1)c1c(Cl)cnc(F)c1Cl]

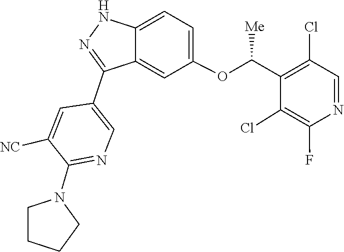 [SMILES: C[C@@H](Oc1ccc2[nH]nc(-c3cnc(N4CCCC4)c(C#N)c3)c2c1)c1c(Cl)cnc(F)c1Cl]

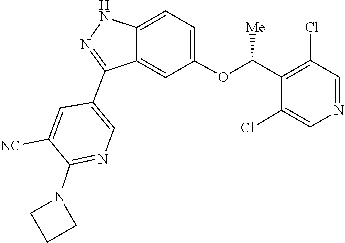 [SMILES: C[C@@H](Oc1ccc2[nH]nc(-c3cnc(N4CCC4)c(C#N)c3)c2c1)c1c(Cl)cncc1Cl]

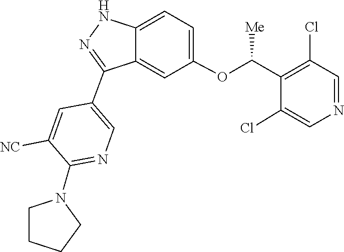 [SMILES: C[C@@H](Oc1ccc2[nH]nc(-c3cnc(N4CCCC4)c(C#N)c3)c2c1)c1c(Cl)cncc1Cl]

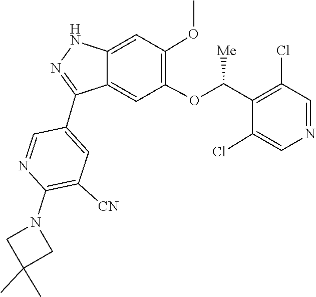 [SMILES: COc1cc2[nH]nc(-c3cnc(N4CC(C)(C)C4)c(C#N)c3)c2cc1O[C@H](C)c1c(Cl)cncc1Cl]

5-[5-[(1R)-1-(3,5-dichloro-
4-pyridyl)ethoxy]-6-
methoxy-1H-indazol-3-yl]-
2-(3,3-dimethylazetidin-1-
yl)pyridine-3-carbonitrile

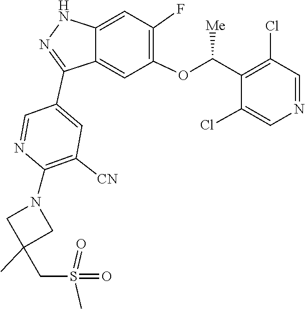 [SMILES: C[C@@H](Oc1cc2c(-c3cnc(N4CC(C)(CS(C)(=O)=O)C4)c(C#N)c3)n[nH]c2cc1F)c1c(Cl)cncc1Cl]

5-[5-[(1R)-1-(3,5-dichloro-
4-pyridyl)ethoxy]-6-fluoro-
1H-indazol-3-yl]-2-[3-
methyl-3-
(methylsulfonylmethyl)
azetidin-1-yl]pyridine-3-
carbonitrile

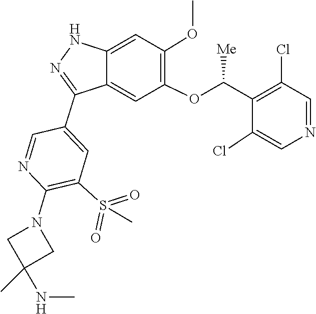 [SMILES: CNC1(C)CN(c2ncc(-c3n[nH]c4cc(OC)c(O[C@H](C)c5c(Cl)cncc5Cl)cc34)cc2S(C)(=O)=O)C1]

1-[5-[5-[(1R)-1-(3,5-
dichloro-4-pyridyl)ethoxy]-
6-methoxy-1H-indazol-3-
yl]-3-methylsulfonyl-2-
pyridyl]-N,3-dimethyl-
azetidin-3-amine

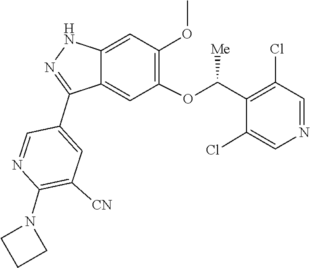 [SMILES: COc1cc2[nH]nc(-c3cnc(N4CCC4)c(C#N)c3)c2cc1O[C@H](C)c1c(Cl)cncc1Cl]

2-(azetidin-1-yl)-5-[5-
[(1R)-1-(3,5-dichloro-4-
pyridyl)ethoxy]-6-
methoxy-1H-indazol-3-
yl]pyridine-3-carbonitrile

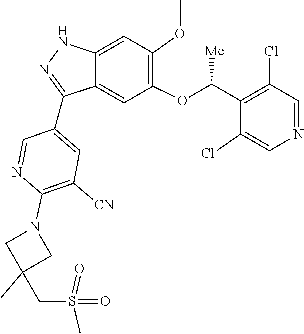 [SMILES: COc1cc2[nH]nc(-c3cnc(N4CC(C)(CS(C)(=O)=O)C4)c(C#N)c3)c2cc1O[C@H](C)c1c(Cl)cncc1Cl]

5-[5-[(1R)-1-(3,5-dichloro-
4-pyridyl)ethoxy]-6-
methoxy-1H-indazol-3-yl]-
2-[3-methyl-3-
(methylsulfonylmethyl)
azetidin-1-yl]pyridine-3-
carbonitrile

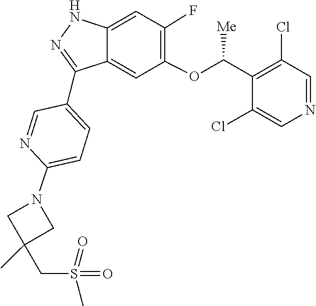 [SMILES: C[C@@H](Oc1cc2c(-c3ccc(N4CC(C)(CS(C)(=O)=O)C4)nc3)n[nH]c2cc1F)c1c(Cl)cncc1Cl]

5-[(1R)-1-(3,5-dichloro-4-
pyridyl)ethoxy]-6-fluoro-3-
[6-[3-methyl-3-
(methylsulfonylmethyl)
azetidin-1-yl]-3-pyridyl]-1H-
indazole

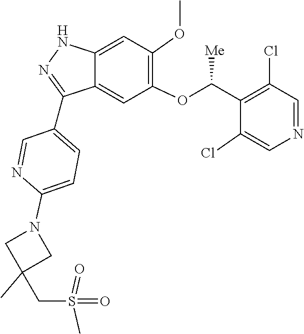 [SMILES: COc1cc2[nH]nc(-c3ccc(N4CC(C)(CS(C)(=O)=O)C4)nc3)c2cc1O[C@H](C)c1c(Cl)cncc1Cl]

5-[(1R)-1-(3,5-dichloro-4-
pyridyl)ethoxy]-6-
methoxy-3-[6-[3-methyl-3-
(methylsulfonylmethyl)
azetidin-1-yl]-3-pyridyl]-1H-
indazole

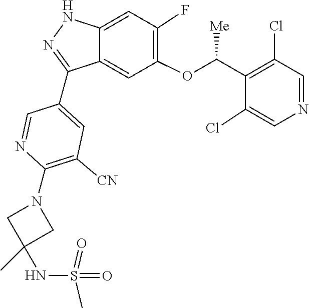 [SMILES: C[C@@H](Oc1cc2c(-c3cnc(N4CC(C)(NS(C)(=O)=O)C4)c(C#N)c3)n[nH]c2cc1F)c1c(Cl)cncc1Cl]

N-[1-[3-cyano-5-[5-[(1R)-
1-(3,5-dichloro-4-
pyridyl)ethoxy]-6-fluoro-
1H-indazol-3-yl]-2-
pyridyl]-3-methyl-azetidin-
3-yl]methanesulfonamide

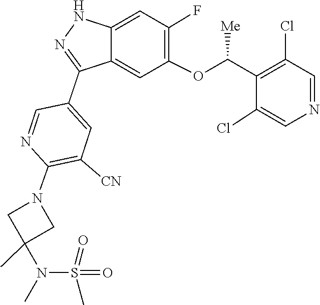 [SMILES: C[C@@H](Oc1cc2c(-c3cnc(N4CC(C)(N(C)S(C)(=O)=O)C4)c(C#N)c3)n[nH]c2cc1F)c1c(Cl)cncc1Cl]

N-[1-[3-cyano-5-[5-[(1R)-
1-(3,5-dichloro-4-
pyridyl)ethoxy]-6-fluoro-
1H-indazol-3-yl]-2-
pyridyl]-3-methyl-azetidin-
3-yl]-N-methyl-
methanesulfonamide

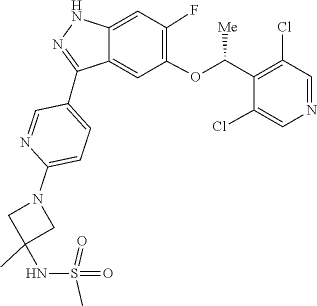 [SMILES: C[C@@H](Oc1cc2c(-c3ccc(N4CC(C)(NS(C)(=O)=O)C4)nc3)n[nH]c2cc1F)c1c(Cl)cncc1Cl]

N-[1-[5-[5-[(1R)-1-(3,5-
dichloro-4-pyridyl)ethoxy]-
6-fluoro-1H-indazol-3-yl]-
2-pyridyl]-3-methyl-
azetidin-3-
yl]methanesulfonamide

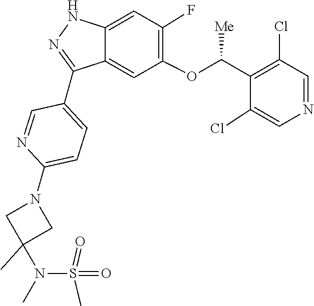 [SMILES: C[C@@H](Oc1cc2c(-c3ccc(N4CC(C)(N(C)S(C)(=O)=O)C4)nc3)n[nH]c2cc1F)c1c(Cl)cncc1Cl]

N-[1-[5-[5-[(1R)-1-(3,5-
dichloro-4-
pyridyl)ethoxy]-6-fluoro-
1H-indazol-3-yl]-2-
pyridyl]-3-methyl-azetidin-
3-yl]-N-methyl-
methanesulfonamide

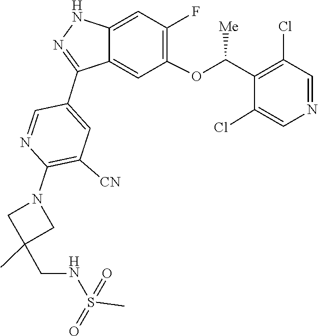 [SMILES: C[C@@H](Oc1cc2c(-c3cnc(N4CC(C)(CNS(C)(=O)=O)C4)c(C#N)c3)n[nH]c2cc1F)c1c(Cl)cncc1Cl]

N-[1-[3-cyano-5-[5-[(1R)-
1-(3,5-dichloro-4-
pyridyl)ethoxy]-6-fluoro-
1H-indazol-3-yl]-2-
pyridyl]-3-methyl-azetidin-
3-
yl]methyl]methanesulfonamide

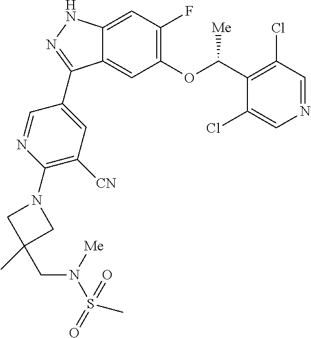 [SMILES: C[C@@H](Oc1cc2c(-c3cnc(N4CC(C)(CN(C)S(C)(=O)=O)C4)c(C#N)c3)n[nH]c2cc1F)c1c(Cl)cncc1Cl]

N-[[1-[3-cyano-5-[5-[(1R)-
1-(3,5-dichloro-4-
pyridyl)ethoxy]-6-fluoro-
1H-indazol-3-yl]-2-
pyridyl]-3-methyl-azetidin-
3-yl]methyl]-N-methyl-
methanesulfonamide

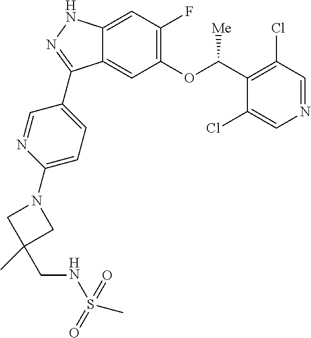 [SMILES: C[C@@H](Oc1cc2c(-c3ccc(N4CC(C)(CNS(C)(=O)=O)C4)nc3)n[nH]c2cc1F)c1c(Cl)cncc1Cl]

N-[[1-[5-[5-[(1R)-1-(3,5-
dichloro-4-
pyridyl)ethoxy]-6-fluoro-
1H-indazol-3-yl]-2-
pyridyl]-3-methyl-azetidin-
3-
yl]methyl]methanesulfonamide

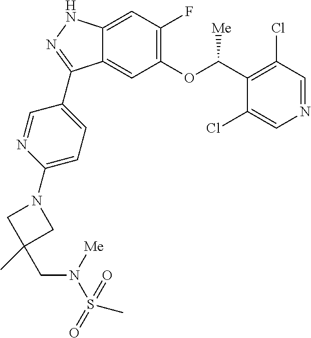 [SMILES: C[C@@H](Oc1cc2c(-c3ccc(N4CC(C)(CN(C)S(C)(=O)=O)C4)nc3)n[nH]c2cc1F)c1c(Cl)cncc1Cl]

N-[[1-[5-[5-[(1R)-1-(3,5-
dichloro-4-pyridyl)ethoxy]-
6-fluoro-1H-indazol-3-yl]-
2-pyridyl]-3-methyl-
azetidin-3-yl]methyl]-N-
methyl-
methanesulfonamide

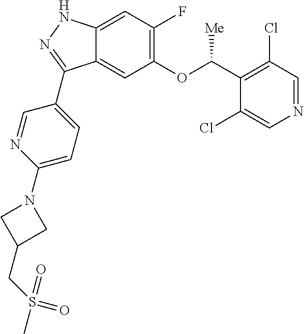 [SMILES: C[C@@H](Oc1cc2c(-c3ccc(N4CC(CS(C)(=O)=O)C4)nc3)n[nH]c2cc1F)c1c(Cl)cncc1Cl]

5-[(1R)-1-(3,5-dichloro-4-
pyridyl)ethoxy]-6-fluoro-3-
[6-[3-
(methylsulfonylmethyl)
azetidin-1-yl]-3-pyridyl]-1H-
indazole

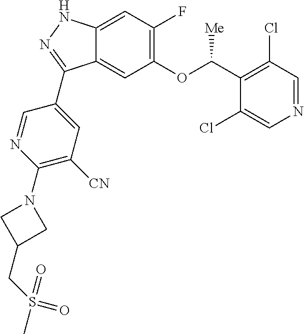 [SMILES: C[C@@H](Oc1cc2c(-c3cnc(N4CC(CS(C)(=O)=O)C4)c(C#N)c3)n[nH]c2cc1F)c1c(Cl)cncc1Cl]

5-[5-[(1R)-1-(3,5-dichloro-
4-pyridyl)ethoxy]-6-fluoro-
1H-indazol-3-yl]-2-[3-
(methylsulfonylmethyl)
azetidin-1-yl]pyridine-3-
carbonitrile

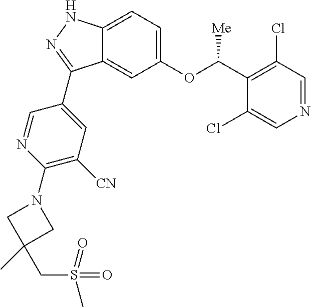 [SMILES: C[C@@H](Oc1ccc2[nH]nc(-c3cnc(N4CC(C)(CS(C)(=O)=O)C4)c(C#N)c3)c2c1)c1c(Cl)cncc1Cl]

5-[5-[(1R)-1-(3,5-dichloro-
4-pyridyl)ethoxy]-1H-
indazol-3-yl]-2-[3-methyl-
3-
(methylsulfonylmethyl)
azetidin-1-yl]pyridine-3-
carbonitrile

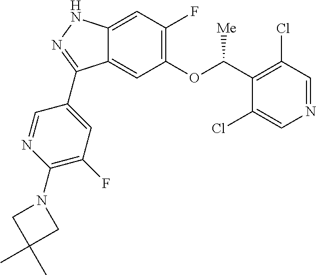 [SMILES: C[C@@H](Oc1cc2c(-c3cnc(N4CC(C)(C)C4)c(F)c3)n[nH]c2cc1F)c1c(Cl)cncc1Cl]

5-[(1R)-1-(3,5-dichloro-4-
pyridyl)ethoxy]-3-[6-(3,3-
dimethylazetidin-1-yl)-5-
fluoro-3-pyridyl]-6-fluoro-
1H-indazole

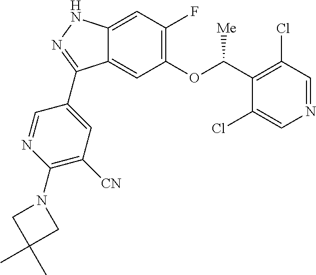 [SMILES: C[C@@H](Oc1cc2c(-c3cnc(N4CC(C)(C)C4)c(C#N)c3)n[nH]c2cc1F)c1c(Cl)cncc1Cl]

5-[5-[(1R)-1-(3,5-dichloro-
4-pyridyl)ethoxy]-6-fluoro-
1H-indazol-3-yl]-2-(3,3-
dimethylazetidin-1-
yl)pyridine-3-carbonitrile

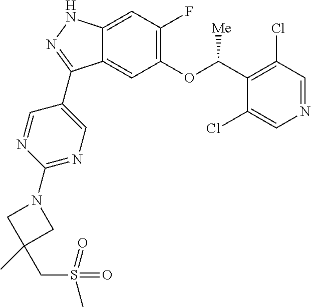 [SMILES: C[C@@H](Oc1cc2c(-c3cnc(N4CC(C)(CS(C)(=O)=O)C4)nc3)n[nH]c2cc1F)c1c(Cl)cncc1Cl]

5-[(1R)-1-(3,5-dichloro-4-
pyridyl)ethoxy]-6-fluoro-3-
[2-[3-methyl-3-
(methylsulfonylmethyl)
azetidin-1-yl]pyrimidin-5-yl]-
1H-indazole

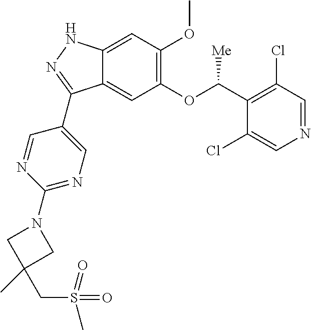 [SMILES: COc1cc2[nH]nc(-c3cnc(N4CC(C)(CS(C)(=O)=O)C4)nc3)c2cc1O[C@H](C)c1c(Cl)cncc1Cl]

5-[(1R)-1-(3,5-dichloro-4-
pyridyl)ethoxy]-6-
methoxy-3-[2-[3-methyl-3-
(methylsulfonylmethyl)
azetidin-1-yl]pyrimidin-5-yl]-
1H-indazole

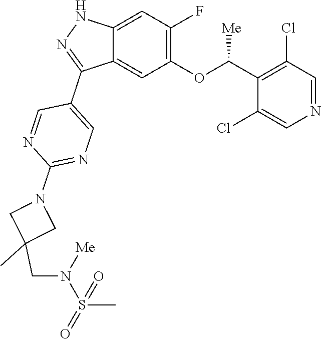 [SMILES: C[C@@H](Oc1cc2c(-c3cnc(N4CC(C)(CN(C)S(C)(=O)=O)C4)nc3)n[nH]c2cc1F)c1c(Cl)cncc1Cl]

N-[[1-[5-[5-[(1R)-1-(3,5-
dichloro-4-
pyridyl)ethoxy]-6-fluoro-
1H-indazol-3-yl]pyrimidin-
2-yl]-3-methyl-azetidin-3-
yl]methyl]-N-methyl-
methanesulfonamide

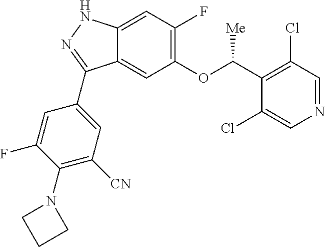 [SMILES: C[C@@H](Oc1cc2c(-c3cc(F)c(N4CCC4)c(C#N)c3)n[nH]c2cc1F)c1c(Cl)cncc1Cl]

2-(azetidin-1-yl)-5-[5-
[(1R)-1-(3,5-dichloro-4-
pyridyl)ethoxy]-6-fluoro-
1H-indazol-3-yl]-3-fluoro-
benzonitrile

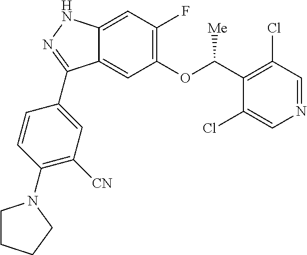 [SMILES: C[C@@H](Oc1cc2c(-c3ccc(N4CCCC4)c(C#N)c3)n[nH]c2cc1F)c1c(Cl)cncc1Cl]

5-[5-[(1R)-1-(3,5-dichloro-
4-pyridyl)ethoxy]-6-fluoro-
1H-indazol-3-yl]-2-
pyrrolidin-1-yl-benzonitrile

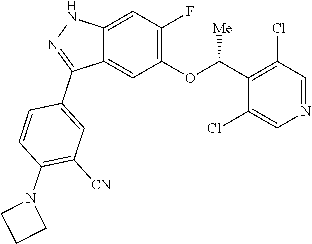 [SMILES: C[C@@H](Oc1cc2c(-c3ccc(N4CCC4)c(C#N)c3)n[nH]c2cc1F)c1c(Cl)cncc1Cl]

2-(azetidin-1-yl)-5-[5-
[(1R)-1-(3,5-dichloro-4-
pyridyl)ethoxy]-6-fluoro-
1H-indazol-3-
yl]benzonitrile

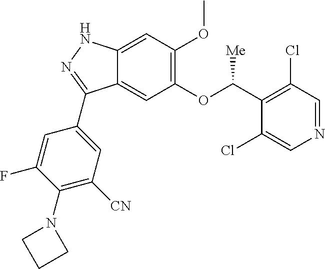 [SMILES: COc1cc2[nH]nc(-c3cc(F)c(N4CCC4)c(C#N)c3)c2cc1O[C@H](C)c1c(Cl)cncc1Cl]

2-(azetidin-1-yl)-5-[5-
[(1R)-1-(3,5-dichloro-4-
pyridyl)ethoxy]-6-
methoxy-1H-indazol-3-yl]-
3-fluoro-benzonitrile

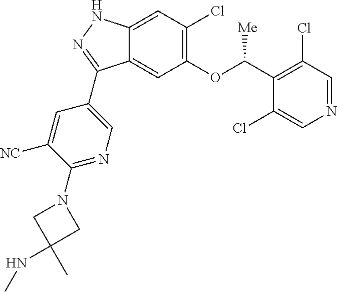 [SMILES: CNC1(C)CN(c2ncc(-c3n[nH]c4cc(Cl)c(O[C@H](C)c5c(Cl)cncc5Cl)cc34)cc2C#N)C1]

5-[6-chloro-5-[(1R)-1-(3,5-
dichloro-4-pyridyl)ethoxy]-
1H-indazol-3-yl]-2-[3-
methyl-3-
(methylamino)azetidin-1-
yl]pyridine-3-carbonitrile

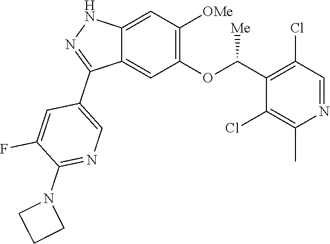 [SMILES: COc1cc2[nH]nc(-c3cnc(N4CCC4)c(F)c3)c2cc1O[C@H](C)c1c(Cl)cnc(C)c1Cl]

3-[6-(azetidin-1-yl)-5-
fluoro-3-pyridyl]-5-[(1R)-
1-(3,5-dichloro-2-methyl-
4-pyridyl)ethoxy]-6-
methoxy-1H-indazole

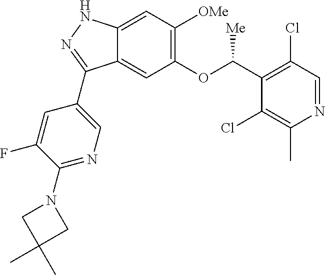 [SMILES: COc1cc2[nH]nc(-c3cnc(N4CC(C)(C)C4)c(F)c3)c2cc1O[C@H](C)c1c(Cl)cnc(C)c1Cl]

5-[(1R)-1-(3,5-dichloro-2-
methyl-4-pyridyl)ethoxy]-
3-[6-(3,3-dimethylazetidin-
1-yl)-5-fluoro-3-pyridyl]-6-
methoxy-1H-indazole

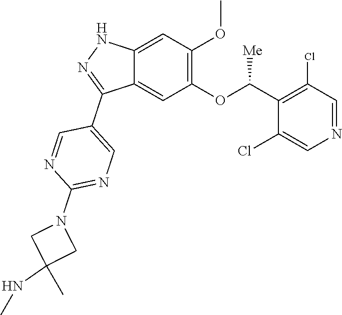 [SMILES: CNC1(C)CN(c2ncc(-c3n[nH]c4cc(OC)c(O[C@H](C)c5c(Cl)cncc5Cl)cc34)cn2)C1]

1-[5-[5-[(1R)-1-(3,5-
dichloro-4-pyridyl)ethoxy]-
6-methoxy-1H-indazol-3-
yl]pyrimidin-2-yl]-N,3-
dimethyl-azetidin-3-amine

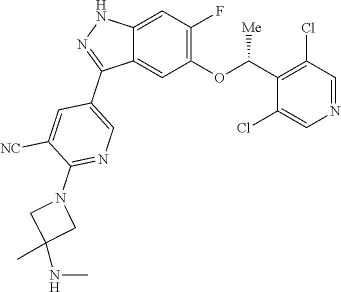 [SMILES: CNC1(C)CN(c2ncc(-c3n[nH]c4cc(F)c(O[C@H](C)c5c(Cl)cncc5Cl)cc34)cc2C#N)C1]

5-[5-[(1R)-1-(3,5-dichloro-
4-pyridyl)ethoxy]-6-fluoro-
1H-indazol-3-yl]-2-[3-
methyl-3-
(methylamino)azetidin-1-
yl]pyridine-3-carbonitrile

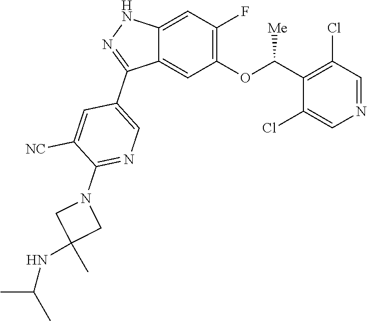 [SMILES: CC(C)NC1(C)CN(c2ncc(-c3n[nH]c4cc(F)c(O[C@H](C)c5c(Cl)cncc5Cl)cc34)cc2C#N)C1]

5-[5-[(1R)-1-(3,5-dichloro-
4-pyridyl)ethoxy]-6-fluoro-
1H-indazol-3-yl]-2-[3-
(isopropylamino)-3-
methyl-azetidin-1-
yl]pyridine-3-carbonitrile

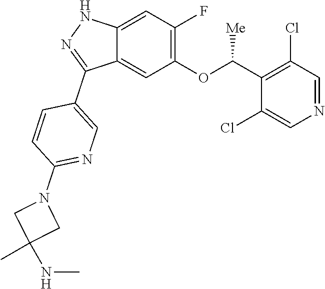 [SMILES: CNC1(C)CN(c2ccc(-c3n[nH]c4cc(F)c(O[C@H](C)c5c(Cl)cncc5Cl)cc34)cn2)C1]

1-[5-[5-[(1R)-1-(3,5-
dichloro-4-pyridyl)ethoxy]-
6-fluoro-1H-indazol-3-yl]-
2-pyridyl]-N,3-dimethyl-
azetidin-3-amine

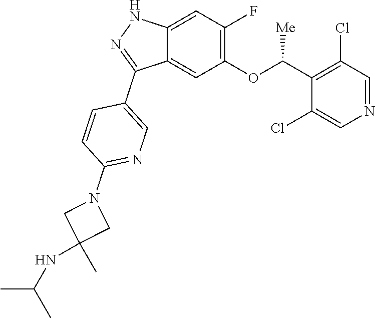 [SMILES: CC(C)NC1(C)CN(c2ccc(-c3n[nH]c4cc(F)c(O[C@H](C)c5c(Cl)cncc5Cl)cc34)cn2)C1]

1-[5-[5-[(1R)-1-(3,5-
dichloro-4-
pyridyl)ethoxy]-6-fluoro-
1H-indazol-3-yl]-2-
pyridyl]-N-isopropyl-3-
methyl-azetidin-3-amine

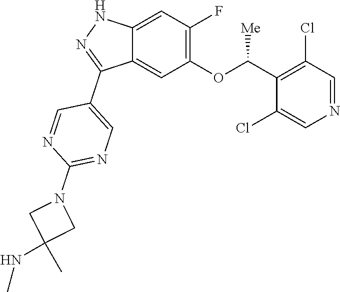 [SMILES: CNC1(C)CN(c2ncc(-c3n[nH]c4cc(F)c(O[C@H](C)c5c(Cl)cncc5Cl)cc34)cn2)C1]

1-[5-[5-[(1R)-1-(3,5-
dichloro-4-pyridyl)ethoxy]-
6-fluoro-1H-indazol-3-
yl]pyrimidin-2-yl]-N,3-
dimethyl-azetidin-3-amine

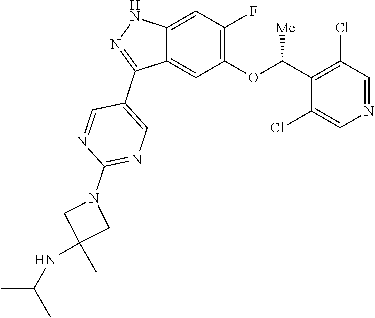 [SMILES: CC(C)NC1(C)CN(c2ncc(-c3n[nH]c4cc(F)c(O[C@H](C)c5c(Cl)cncc5Cl)cc34)cn2)C1]

1-[5-[5-[(1R)-1-(3,5-
dichloro-4-pyridyl)ethoxy]-
6-fluoro-1H-indazol-3-
yl]pyrimidin-2-yl]-N-
isopropyl-3-methyl-
azetidin-3-amine

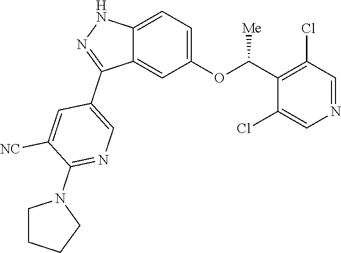 [SMILES: C[C@@H](Oc1ccc2[nH]nc(-c3cnc(N4CCCC4)c(C#N)c3)c2c1)c1c(Cl)cncc1Cl]

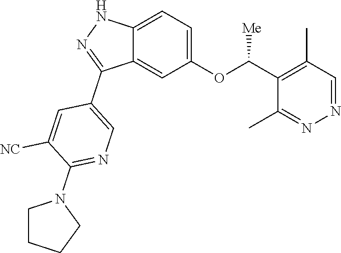 [SMILES: Cc1cnnc(C)c1[C@@H](C)Oc1ccc2[nH]nc(-c3cnc(N4CCCC4)c(C#N)c3)c2c1]

5-[5-[(1R)-1-(3,5-
dimethylpyridazin-4-
yl)ethoxy]-1H-indazol-3-
yl]-2-pyrrolidin-1-yl-
pyridine-3-carbonitrile

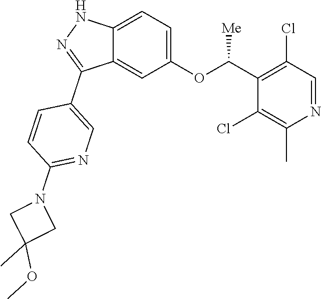 [SMILES: COC1(C)CN(c2ccc(-c3n[nH]c4ccc(O[C@H](C)c5c(Cl)cnc(C)c5Cl)cc34)cn2)C1]

5-[(1R)-1-(3,5-dichloro-2-
methyl-4-pyridyl)ethoxy]-
3-[6-(3-methoxy-3-methyl-
azetidin-1-yl)-3-pyridyl]-
1H-indazole

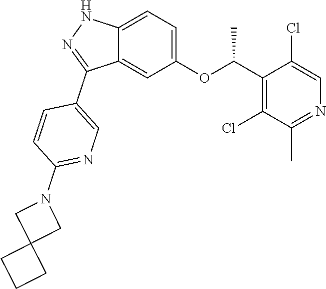 [SMILES: Cc1ncc(Cl)c([C@@H](C)Oc2ccc3[nH]nc(-c4ccc(N5CC6(CCC6)C5)nc4)c3c2)c1Cl]

3-[6-(2-azaspiro[3.3]-
heptan-2-yl)-3-pyridyl]-5-
[(1R)-1-(3,5-dichloro-2-
methyl-4-pyridyl)ethoxy]-
1H-indazole

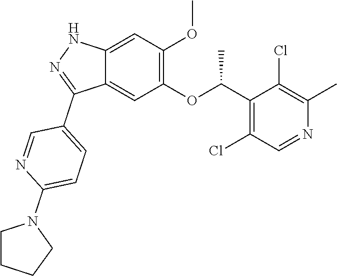 [SMILES: COc1cc2[nH]nc(-c3ccc(N4CCCC4)nc3)c2cc1O[C@H](C)c1c(Cl)cnc(C)c1Cl]

5-[(1R)-1-(3,5-dichloro-2-
methyl-4-pyridyl)ethoxy]-
6-methoxy-3-(6-pyrrolidin-
1-yl-3-pyridyl)-1H-
indazole

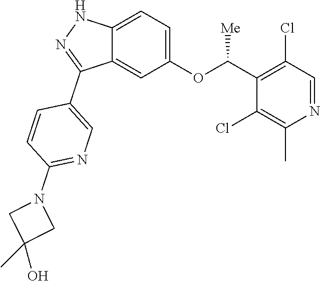 [SMILES: Cc1ncc(Cl)c([C@@H](C)Oc2ccc3[nH]nc(-c4ccc(N5CC(C)(O)C5)nc4)c3c2)c1Cl]

1-[5-[5-[(1R)-1-(3,5-
dichloro-2-methyl-4-
pyridyl)ethoxy]-1H-
indazol-3-yl]-2-pyridyl]-3-
methyl-azetidin-3-ol

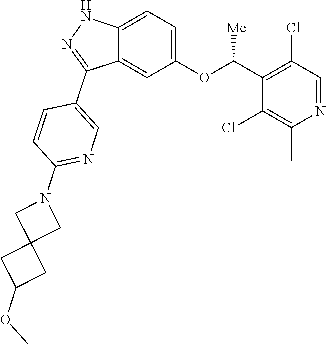 [SMILES: COC1CC2(C1)CN(c1ccc(-c3n[nH]c4ccc(O[C@H](C)c5c(Cl)cnc(C)c5Cl)cc34)cn1)C2]

5-[(1R)-1-(3,5-dichloro-2-
methyl-4-pyridyl)ethoxy]-
3-[6-(6-methoxy-2-
azaspiro[3.3]heptan-2-yl)-
3-pyridyl]-1H-indazole

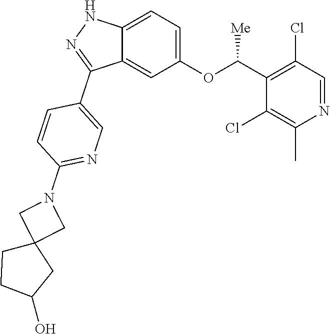 [SMILES: Cc1ncc(Cl)c([C@@H](C)Oc2ccc3[nH]nc(-c4ccc(N5CC6(CCC(O)C6)C5)nc4)c3c2)c1Cl]

2-[5-[5-[(1R)-1-(3,5-
dichloro-2-methyl-4-
pyridyl)ethoxy]-1H-
indazol-3-yl]-2-pyridyl]-2-
azaspiro[3.4]octan-6-ol

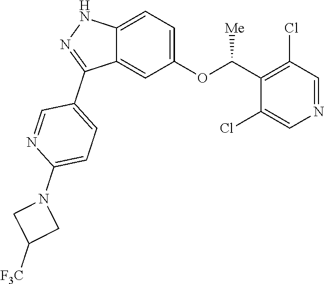 [SMILES: C[C@@H](Oc1ccc2[nH]nc(-c3ccc(N4CC(C(F)(F)F)C4)nc3)c2c1)c1c(Cl)cncc1Cl]

(R)-5-(1-(3,5-
dichloropyridin-4-
yl)ethoxy)-3-(6-(3-
(trifluoromethyl)azetidin-1-
yl)pyridin-3-yl)-1H-
indazole

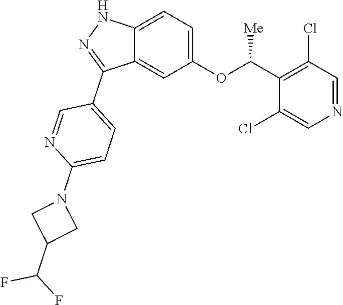 [SMILES: C[C@@H](Oc1ccc2[nH]nc(-c3ccc(N4CC(C(F)F)C4)nc3)c2c1)c1c(Cl)cncc1Cl]

(R)-5-(1-(3,5-
dichloropyridin-4-
yl)ethoxy)-3-(6-(3-
(difluoromethyl)azetidin-1-
yl)pyridin-3-yl)-1H-
indazole

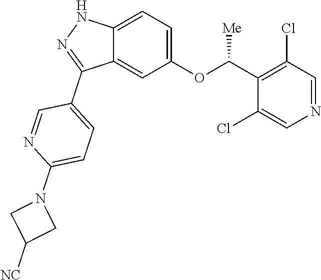 [SMILES: C[C@@H](Oc1ccc2[nH]nc(-c3ccc(N4CC(C#N)C4)nc3)c2c1)c1c(Cl)cncc1Cl]

(R)-1-(5-(5-(1-(3,5-
dichloropyridin-4-
yl)ethoxy)-1H-indazol-3-
yl)pyridin-2-yl)azetidine-3-
carbonitrile

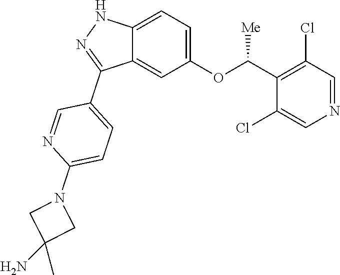 [SMILES: C[C@@H](Oc1ccc2[nH]nc(-c3ccc(N4CC(C)(N)C4)nc3)c2c1)c1c(Cl)cncc1Cl]

(R)-1-(5-(5-(1-(3,5-
dichloropyridin-4-
yl)ethoxy)-1H-indazol-3-
yl)pyridin-2-yl)-3-
methylazetidin-3-amine

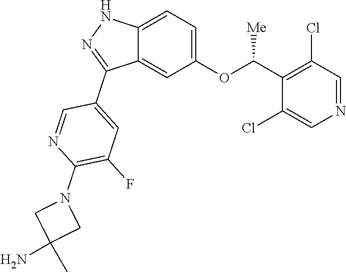 [SMILES: C[C@@H](Oc1ccc2[nH]nc(-c3cnc(N4CC(C)(N)C4)c(F)c3)c2c1)c1c(Cl)cncc1Cl]

(R)-1-(5-(5-(1-(3,5-
dichloropyridin-4-
yl)ethoxy)-1H-indazol-3-
yl)-3-fluoropyridin-2-yl)-3-
methylazetidin-3-amine

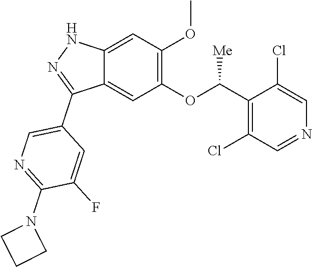 [SMILES: COc1cc2[nH]nc(-c3cnc(N4CCC4)c(F)c3)c2cc1O[C@H](C)c1c(Cl)cncc1Cl]

(R)-3-(6-(azetidin-1-yl)-5-
fluoropyridin-3-yl)-5-(1-
(3,5-dichloropyridin-4-
yl)ethoxy)-6-methoxy-1H-
indazole

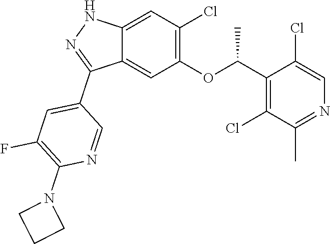 [SMILES: Cc1ncc(Cl)c([C@@H](C)Oc2cc3c(-c4cnc(N5CCC5)c(F)c4)n[nH]c3cc2Cl)c1Cl]

3-[6-(azetidin-1-yl)-5-
fluoro-3-pyridyl]-6-chloro-
5-[(1R)-1-(3,5-dichloro-2-
methyl-4-pyridyl)ethoxy]-
1H-indazole

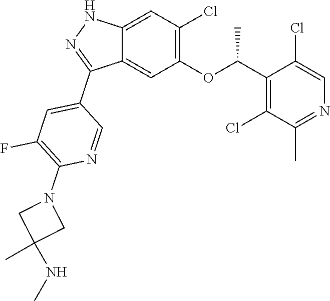 [SMILES: CNC1(C)CN(c2ncc(-c3n[nH]c4cc(Cl)c(O[C@H](C)c5c(Cl)cnc(C)c5Cl)cc34)cc2F)C1]

1-[5-[6-chloro-5-[(1R)-1-
(3,5-dichloro-2-methyl-4-
pyridyl)ethoxy]-1H-
indazol-3-yl]-3-fluoro-2-
pyridyl]-N,3-dimethyl-
azetidin-3-amine

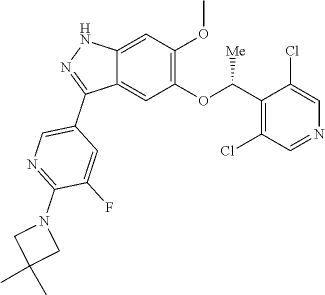 [SMILES: COc1cc2[nH]nc(-c3cnc(N4CC(C)(C)C4)c(F)c3)c2cc1O[C@H](C)c1c(Cl)cncc1Cl]

(R)-5-(1-(3,5-
dichloropyridin-4-
yl)ethoxy)-3-(6-(3,3-
dimethylazetidin-1-yl)-5-
fluoropyridin-3-yl)-6-
methoxy-1H-indazole

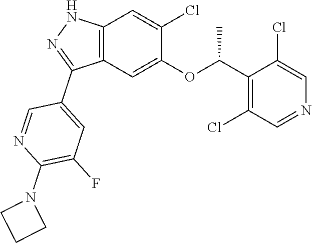 [SMILES: C[C@@H](Oc1cc2c(-c3cnc(N4CCC4)c(F)c3)n[nH]c2cc1Cl)c1c(Cl)cncc1Cl]

(R)-3-(6-(azetidin-1-yl)-5-
fluoropyridin-3-yl)-6-
chloro-5-(1-(3,5-
dichloropyridin-4-
yl)ethoxy)-1H-indazole

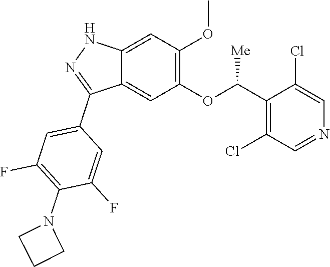 [SMILES: COc1cc2[nH]nc(-c3cc(F)c(N4CCC4)c(F)c3)c2cc1O[C@H](C)c1c(Cl)cncc1Cl]

(R)-3-(4-(azetidin-1-yl)-
3,5-difluorophenyl)-5-(1-
(3,5-dichloropyridin-4-
yl)ethoxy)-6-methoxy-1H-
indazole

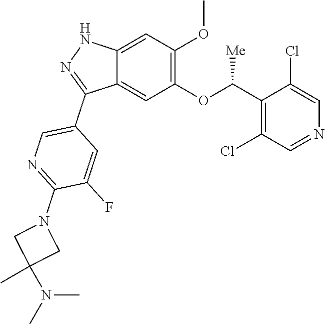 [SMILES: COc1cc2[nH]nc(-c3cnc(N4CC(C)(N(C)C)C4)c(F)c3)c2cc1O[C@H](C)c1c(Cl)cncc1Cl]

(R)-1-(5-(5-(1-(3,5-
dichloropyridin-4-
yl)ethoxy)-6-methoxy-1H-
indazol-3-yl)-3-
fluoropyridin-2-yl)-N,N,3-
trimethylazetidin-3-amine

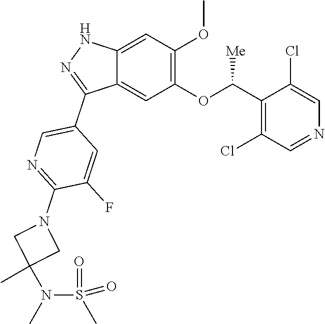 [SMILES: COc1cc2[nH]nc(-c3cnc(N4CC(C)(N(C)S(C)(=O)=O)C4)c(F)c3)c2cc1O[C@H](C)c1c(Cl)cncc1Cl]

(R)-N-(1-(5-(5-(1-(3,5-
dichloropyridin-4-
yl)ethoxy)-6-methoxy-1H-
indazol-3-yl)-3-
fluoropyridin-2-yl)-3-
methylazetidin-3-yl)-N-
methylmethanesulfonamide

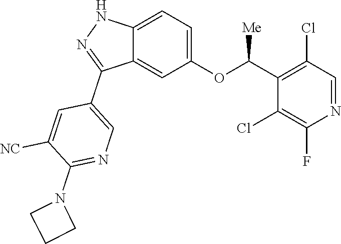 [SMILES: C[C@H](Oc1ccc2[nH]nc(-c3cnc(N4CCC4)c(C#N)c3)c2c1)c1c(Cl)cnc(F)c1Cl]

(S)-2-(azetidin-1-yl)-5-(5-
(1-(3,5-dichloro-2-
fluoropyridin-4-yl)ethoxy)-
1H-indazol-3-
yl)nicotinonitrile

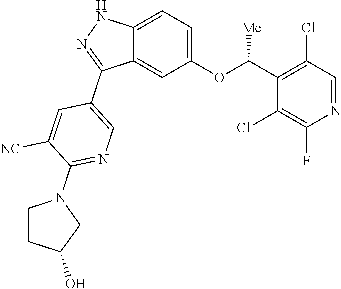 [SMILES: C[C@@H](Oc1ccc2[nH]nc(-c3cnc(N4CC[C@@H](O)C4)c(C#N)c3)c2c1)c1c(Cl)cnc(F)c1Cl]

5-(5-((R)-1-(3,5-dichloro-
2-fluoropyridin-4-
yl)ethoxy)-1H-indazol-3-
yl)-2-((R)-3-
hydroxypyrrolidin-1-
yl)nicotinonitrile

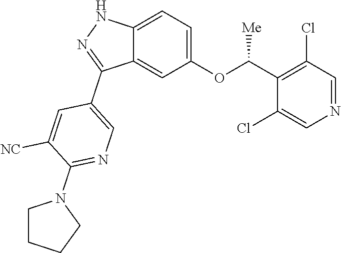 [SMILES: C[C@@H](Oc1ccc2[nH]nc(-c3cnc(N4CCCC4)c(C#N)c3)c2c1)c1c(Cl)cncc1Cl]

5-[5-[(1R)-1-(3,5-dichloro-
4-pyridyl)ethoxy]-1H-
indazol-3-yl]-2-pyrrolidin-
1-yl-pyridine-3-carbonitrile

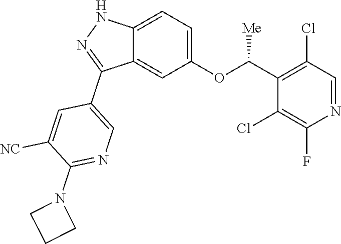 [SMILES: C[C@@H](Oc1ccc2[nH]nc(-c3cnc(N4CCC4)c(C#N)c3)c2c1)c1c(Cl)cnc(F)c1Cl]

(R)-2-(azetidin-1-yl)-5-(5-
(1-(3,5-dichloro-2-
fluoropyridin-4-yl)ethoxy)-
1H-indazol-3-
yl)nicotinonitrile

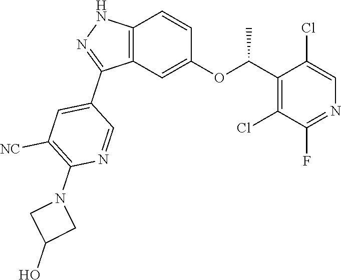 [SMILES: C[C@@H](Oc1ccc2[nH]nc(-c3cnc(N4CC(O)C4)c(C#N)c3)c2c1)c1c(Cl)cnc(F)c1Cl]

(R)-5-(5-(1-(3,5-dichloro-
2-fluoropyridin-4-
yl)ethoxy)-1H-indazol-3-
yl)-2-(3-hydroxyazetidin-1-
yl)nicotinonitrile

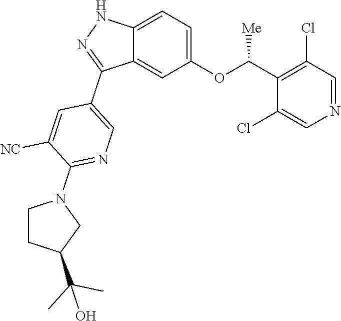 [SMILES: C[C@@H](Oc1ccc2[nH]nc(-c3cnc(N4CC[C@H](C(C)(C)O)C4)c(C#N)c3)c2c1)c1c(Cl)cncc1Cl]

5-[5-[(1R)-1-(3,5-dichloro-
4-pyridyl)ethoxy]-1H-
indazol-3-yl]-2-[(3S)-3-(1-
hydroxy-1-methyl-
ethyl)pyrrolidin-1-
yl]pyridine-3-carbonitrile

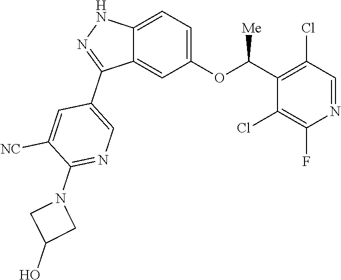 [SMILES: C[C@H](Oc1ccc2[nH]nc(-c3cnc(N4CC(O)C4)c(C#N)c3)c2c1)c1c(Cl)cnc(F)c1Cl]

(S)-5-(5-(1-(3,5-dichloro-
2-fluoropyridin-4-
yl)ethoxy)-1H-indazol-3-
yl)-2-(3-hydroxyazetidin-
1-yl)nicotinonitrile

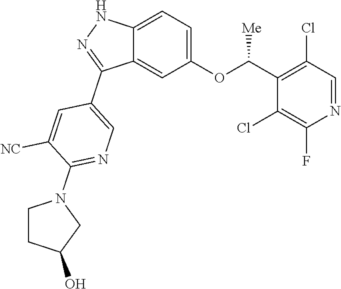 [SMILES: C[C@@H](Oc1ccc2[nH]nc(-c3cnc(N4CC[C@H](O)C4)c(C#N)c3)c2c1)c1c(Cl)cnc(F)c1Cl]

5-(5-((R)-1-(3,5-dichloro-
2-fluoropyridin-4-
yl)ethoxy)-1H-indazol-3-
yl)-2-((S)-3-
hydroxypyrrolidin-1-
yl)nicotinonitrile

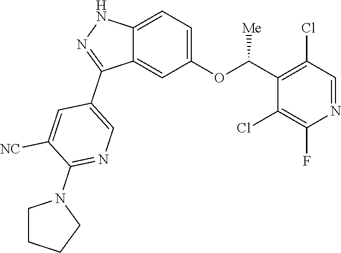 [SMILES: C[C@@H](Oc1ccc2[nH]nc(-c3cnc(N4CCCC4)c(C#N)c3)c2c1)c1c(Cl)cnc(F)c1Cl]

(R)-5-(5-(1-(3,5-dichloro-
2-fluoropyridin-4-
yl)ethoxy)-1H-indazol-3-
yl)-2-(pyrrolidin-1-
yl)nicotinonitrile

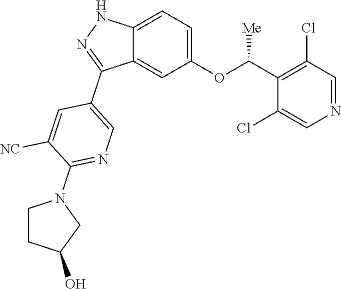 [SMILES: C[C@@H](Oc1ccc2[nH]nc(-c3cnc(N4CC[C@H](O)C4)c(C#N)c3)c2c1)c1c(Cl)cncc1Cl]

5-[5-[(1R)-1-(3,5-dichloro-
4-pyridyl)ethoxy]-1H-
indazol-3-yl]-2-[(3S)-3-
hydroxypyrrolidin-1-
yl]pyridine-3-carbonitrile

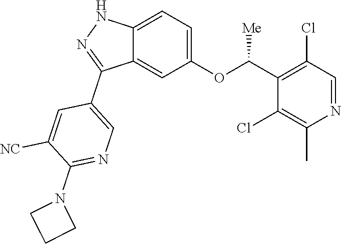 [SMILES: Cc1ncc(Cl)c([C@@H](C)Oc2ccc3[nH]nc(-c4cnc(N5CCC5)c(C#N)c4)c3c2)c1Cl]

2-(azetidin-1-yl)-5-[5-
[(1R)-1-(3,5-dichloro-2-
methyl-4-pyridyl)ethoxy]-
1H-indazol-3-yl]pyridine-
3-carbonitrile

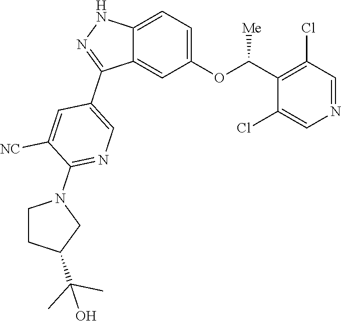 [SMILES: C[C@@H](Oc1ccc2[nH]nc(-c3cnc(N4CC[C@@H](C(C)(C)O)C4)c(C#N)c3)c2c1)c1c(Cl)cncc1Cl]

5-[5-[(1R)-1-(3,5-dichloro-
4-pyridyl)ethoxy]-1H-
indazol-3-yl]-2-[(3R)-3-(1-
hydroxy-1-methyl-
ethyl)pyrrolidin-1-
yl]pyridine-3-carbonitrile

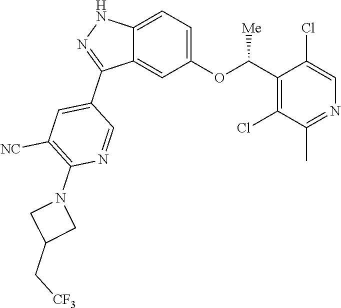 [SMILES: Cc1ncc(Cl)c([C@@H](C)Oc2ccc3[nH]nc(-c4cnc(N5CC(CC(F)(F)F)C5)c(C#N)c4)c3c2)c1Cl]

5-[5-[(1R)-1-(3,5-dichloro-
2-methyl-4-
pyridyl)ethoxy]-1H-
indazol-3-yl]-2-[3-(2,2,2-
trifluoroethyl)azetidin-1-
yl]pyridine-3-carbonitrile

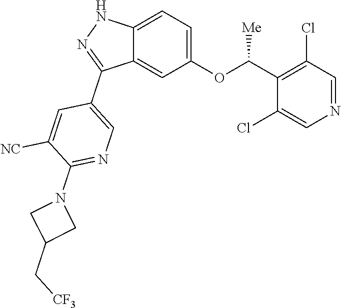 [SMILES: C[C@@H](Oc1ccc2[nH]nc(-c3cnc(N4CC(CC(F)(F)F)C4)c(C#N)c3)c2c1)c1c(Cl)cncc1Cl]

5-[5-[(1R)-1-(3,5-dichloro-
4-pyridyl)ethoxy]-1H-
indazol-3-yl]-2-[3-(2,2,2-
trifluoroethyl)azetidin-1-
yl]pyridine-3-carbonitrile

In some aspects, the disclosure is directed to the compounds shown in the Examples below, or pharmaceutically acceptable salts thereof.

References herein to formula (I) or subgenera thereof are meant to encompass the identified formula and any subgenera of those formula disclosed herein. For example, references to formula (I) also encompass subgenera formula IA, IA-1-1, IA-1, IA-2, IA-3-1, IA-3, IA-4-1, IA-4, IA-5, IA-6, TB.

Stereoisomers of compounds of formula (I) are also contemplated by the present disclosure. Thus, the disclosure encompasses all stereoisomers and constitutional isomers of any compound disclosed or claimed herein, including all enantiomers and diastereomers, or mixtures thereof.

Pharmaceutically acceptable salts and solvates of the compounds of formula (I) are also within the scope of the disclosure.

It is to be appreciated that certain features of the invention which are, for clarity, described herein in the context of separate embodiments, may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. While an embodiment may be described as part of a series of steps or part of a more general structure, each said step may also be considered an independent embodiment in itself, combinable with others.

### Pharmaceutical Compositions and Methods of Administration

The subject pharmaceutical compositions are typically formulated to provide a therapeutically effective amount of a compound of the present disclosure as the active ingredient, or a pharmaceutically acceptable salt, ester, prodrug, solvate, hydrate or derivative thereof. In some embodiments, the pharmaceutical compositions contain a compound of the present disclosure or a pharmaceutically acceptable salt thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered alone or in combination with one or more other agents, which are also typically administered in the form of pharmaceutical compositions. Where desired, the one or more compounds of the invention and other agent(s) may be mixed into a preparation or both components may be formulated into separate preparations to use them in combination separately or at the same time.

In some embodiments, the concentration of one or more compounds provided in the pharmaceutical compositions of the present invention is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v or v/v.

In some embodiments, the concentration of one or more compounds of the invention is greater than 90%, 80%, 70%, 60%, 50%, 40%, 30%, 20%, 19.75%, 19.50%, 19.25%, 19%, 18.75%, 18.50%, 18.25% 18%, 17.75%, 17.50%, 17.25% 17%, 16.75%, 16.50%, 16.25%, 16%, 15.75%, 15.50%, 15.25% 15%, 14.75%, 14.50%, 14.25% 14%, 13.75%, 13.50%, 13.25%, 13%, 12.75%, 12.50%, 12.25%, 12%, 11.75%, 11.50%, 11.25% 11%, 10.75%, 10.50%, 10.25% 10%, 9.75%, 9.50%, 9.25%, 9%, 8.75%, 8.50%, 8.25% 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v, or v/v.

In some embodiments, the concentration of one or more compounds of the invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds of the invention is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more compounds of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g (or a number in the range defined by and including any two numbers above).

In some embodiments, the amount of one or more compounds of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g (or a number in the range defined by and including any two numbers above).

In some embodiments, the amount of one or more compounds of the invention is in the range of 0.0001-10 g, 0.0005-9 g, 0.001-8 g, 0.005-7 g, 0.01-6 g, 0.05-5 g, 0.1-4 g, 0.5-4 g, or 1-3 g.

In some embodiments, the compounds according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day. The exact dosage will depend upon the route of administration, the form in which the compound is administered, the subject to be treated, the body weight of the subject to be treated, and the preference and experience of the attending physician.

Unless otherwise noted, the amounts of the compounds described herein are set forth on a free base basis. That is, the amounts indicate that amount of the compound administered, exclusive of, for example, solvent (such as in solvates) or counterions (such as in pharmaceutically acceptable salts).

Described below are non-limiting exemplary pharmaceutical compositions and methods for preparing the same.

### Pharmaceutical Compositions for Oral Administration

In some embodiments, the invention provides a pharmaceutical composition for oral administration containing a compound of the invention, and a pharmaceutical excipient suitable for oral administration.

In some embodiments, the invention provides a solid pharmaceutical composition for oral administration containing: (i) an effective amount of a compound of the invention; optionally (ii) an effective amount of a second agent; and (iii) a pharmaceutical excipient suitable for oral administration. In some embodiments, the composition further contains: (iv) an effective amount of a third agent.

In some embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof, polyoxyethylated vitamins and derivatives thereof, polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-lOoleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, F-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, F-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25%, 50%, 100%, or up to about 200%> by weight, based on the combined weight of the drug, and other excipients. If desired, very small amounts of solubilizer may also be used, such as 5%>, 2%>, 1%) or even less. Typically, the solubilizer may be present in an amount of about 1%> to about 100%, more typically about 5%> to about 25%> by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Pharmaceutical Compositions for Injection

In some embodiments, the invention provides a pharmaceutical composition for injection containing a compound of the present invention and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Pharmaceutical compositions for topical (e.g. transdermal) delivery.

In some embodiments, the invention provides a pharmaceutical composition for transdermal delivery containing a compound of the present invention and a pharmaceutical excipient suitable for transdermal delivery.

Compositions of the present invention can be formulated into preparations in solid, semisolid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation.

Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of a compound of the present invention in controlled amounts, either with or without another agent. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

### Pharmaceutical Compositions for Inhalation

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

### Other Pharmaceutical Compositions

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Administration of the compounds or pharmaceutical composition of the present invention can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g. transdermal application), rectal administration, via local delivery by catheter or stent or through inhalation. Compounds can also be administered intraadiposally or intrathecally.

The amount of the compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g. by dividing such larger doses into several small doses for administration throughout the day.

In some embodiments, a compound of the invention is administered in a single dose.

Typically, such administration will be by injection, e.g., intravenous injection, in order to introduce the agent quickly. However, other routes may be used as appropriate. A single dose of a compound of the invention may also be used for treatment of an acute condition.

In some embodiments, a compound of the invention is administered in multiple doses. Dosing may be about once, twice, three times, four times, five times, six times, or more than six times per day. Dosing may be about once a month, once every two weeks, once a week, or once every other day. In another embodiment a compound of the invention and another agent are administered together about once per day to about 6 times per day. In another embodiment the administration of a compound of the invention and an agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of the invention may continue as long as necessary. In some embodiments, a compound of the invention is administered for more than 1, 2, 3, 4, 5, 6, 7, 14, or 28 days. In some embodiments, a compound of the invention is administered for less than 28, 14, 7, 6, 5, 4, 3, 2, or 1 day. In some embodiments, a compound of the invention is administered chronically on an ongoing basis, e.g., for the treatment of chronic effects.

An effective amount of a compound of the invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g. PEO-PLLA); polydimethylsiloxane, poly(ethylene-vinylacetate), acrylate-based polymers or copolymers (e.g. polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. Compounds of the invention may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. Compounds of the invention may additionally be administered intravascularly from a balloon used during angioplasty. Extravascular administration of the compounds via the pericard or via advential application of formulations of the invention may also be performed to decrease restenosis.

A variety of stent devices which may be used as described are disclosed, for example, in the following references, all of which are hereby incorporated by reference: U.S. Pat. Nos. 5,451,233; 5,040,548; 5,061,273; 5,496,346; 5,292,331; 5,674,278; 3,657,744; 4,739,762; 5,195,984; 5,292,331; 5,674,278; 5,879,382; 6,344,053.

The compounds of the invention may be administered in dosages. It is known in the art that due to intersubject variability in compound pharmacokinetics, individualization of dosing regimen is necessary for optimal therapy. Dosing for a compound of the invention may be found by routine experimentation in light of the instant disclosure.

When a compound of the invention is administered in a composition that comprises one or more agents, and the agent has a shorter half-life than the compound of the invention unit dose forms of the agent and the compound of the invention may be adjusted accordingly.

The subject pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compound in sterile aqueous solutions, for example, aqueous propylene glycol or dextrose solutions. Such dosage forms can be suitably buffered, if desired.

### Methods of Use

The FGFR receptors (FGFR1, FGFR2, FGFR3, and FGFR4) share several structural features in common, including three extracellular immunoglobulin-like (Ig) domains, a hydrophobic transmembrane domain, and an intracellular tyrosine kinase domain split by a kinase insert domain, followed by a cytoplasmic c-terminal tail (Johnson et al., Adv. Cancer Res. 60:1-40, 1993; and Wilkie et al., Curr. Biol. 5:500-507, 1995). In FGFR1, the kinase insert domain spans positions 582 to 595 of the alpha Al isoform of FGFR1. In FGFR2, the kinase insert domain spans positions 585 to 598 of the FGFR2 Ille isoform. In FGFR3, the kinase insert domain spans positions 576 to 589 of the FGFR3 Ille isoform. In FGFR4, the kinase insert domain spans positions 571 to 584 of FGFR4 isoform 1. The c-terminal tail of FGFRs begins following the end of the tyrosine kinase domain and extends to the c-terminus of the protein. Several isoforms of each FGFR have been identified and are the result of alternative splicing of their mRNAs (Johnson et al., Mol. Cell. Biol. 11:4627-4634, 1995; and Chellaiah et al., J. Biol. Chem. 269:11620-11627, 1994).

A few of the receptor variants that result from this alternative splicing have different ligand binding specificities and affinities (Zimmer et al., J. Biol. Chem. 268:7899-7903, 1993; Cheon et al., Proc. Natl. Acad. Sci. U.S.A. 91:989-993, 1994; and Miki et al., Proc. Natl. Acad. Sci. U.S.A. 89:246-250, 1992). Protein sequences for FGFR proteins and nucleic acids encoding FGFR proteins are known in the art. Signaling by FGFRs regulates key biological processes including cell proliferation, survival, migration, and differentiation. Dysregulation of a FGFR gene, a FGFR protein, or expression or activity, or level of the same, has been associated with many types of cancer. For example, dysregulation of FGFRs can occur by multiple mechanisms, such as FGFR gene overexpression, FGFR gene amplification, activating mutations (e.g., point mutations or truncations), and chromosomal rearrangements that lead to FGFR fusion proteins. Dysregulation of a FGFR gene, a FGFR protein, or expression or activity, or level of the same, can result in (or cause in part) the development of a variety of different FGFR-associated cancers.

FGFR fusion proteins are known in the art. See, e.g., Baroy et al., PloS One; 11(9):e0163859. doi: 10.1371/journal.pone.0163859, 2016; Ren et al., Int. J. Cancer, 139(4):836-40, 2016; Marchwicka et al., Cell Biosci., 6:7. doi: 10.1186/s13578-016-0075-9, 2016; PCT Patent Application Publication No. WO 2014/071419A2; U.S. Patent Application Publication No. 2015/0366866A1; PCT Patent Application Publication No. WO 2016/084883A1; PCT Patent Application Publication No. WO 2016/030509A1; PCT Patent Application Publication No. WO 2015/150900A2; PCT Patent Application Publication No. WO 2015/120094A2; Kasaian et al., BMC Cancer., 15:984, 2015; Vakil et al., Neuro-Oncology, 18:Supp. Supplement 3, pp. iii93. Abstract Number: LG-64, 17^{th} International Symposium on Pediatric Neuro-Oncology, Liverpool, United Kingdom, 2016; Astsaturov et al., Journal of Clinical Oncology, 34:Supp. Supplement 15, Abstract Number: 11504, 2016 Annual Meeting of the American Society of Clinical Oncology, Chicago, IL; Heinrich et al., Journal of Clinical Oncology, 34:Supp. Supplement 15, Abstract Number: 11012, 2016 Annual Meeting of the American Society of Clinical Oncology, Chicago, IL; Hall et al., Molecular Cancer Therapeutics, Vol. 14, No. 12, Supp. 2, Abstract Number: B151, AACR-NCI-EORTC International Conference: Molecular Targets and Cancer Therapeutics, 2015; Reuther et al., Journal of Molecular Diagnostics, Vol. 17, No. 6, pp. 813, Abstract Number: ST02, 2015 Annual Meeting of the Association for Molecular Pathology, Austin, TX; Moeini et al., Clin. Cancer. Res., 22(2):291-300, 2016; Schrock et al, J Thorac. Oneal. pii S1556-0864(18)30674-9, 2018. doi: 10.1016/j.jtho.2018.05.027; Pekmezci et al, Acta Nurotapho. Commun. 6(1):47. doi: 10.1186/s40478-018-0551-z; Lowery et al. Clin Cancer Res. pii: clincanres.0078.2018. doi: 10.1158/1078-0432.CCR-18-0078; Ryland et al. J Clin Patho pii: jclinpath-2018-205195, 2018. doi: 10.1136/jclinpath-2018-205195; Ferguson et al. J Neuropatho/Exp Neural 77(6):437-442, 2018. doi: 10.1093/jnen/nly022; Wu et al, BMC Cancer 18(1):343, 2018. doi: 10.1186/s12885-018-4236-6; Shibata et al, Cancer Sci 109(5):1282-1291, 2018. doi: 10.1111/cas.13582; Papdopoulos et al, Br J Cancer, 1117(11):1592-1599, 2017. doi: 10.1038/bjc.2017.330; Hall et al, PLoS One, 11(9):e1062594, 2016. doi: 10.1371/journal.pone.0162594; Johnson et al, Oncologist, 22(12):1478-1490, 2017. doi: 10.1634/theoncologist.2017-0242; Yang et al, Am J Hum Genet, 98(5):843-856, 2016. doi: 10.1016/j.ajhg.2016.03.017; U.S. Patent Application Publication No. 2013/009621; Babina and Turner, Nat Rev Cancer 17(5):318-332, 2017. doi: 10.1038/nrc.2017.8; Ryland et al, J Clin Patho., 2018 May 14. pii: jclinpath-2018-205195. doi: 10.1136/jclinpath-2018-205195; Kumar et al, Am J Clin Patho. 143(5):738-748, 2015. doi: 10.1309/AJCPUD6W1JLQQMNA; Grand et al, Genes Chromosomes Cancer40(1):78-83, 2004. doi: 10.1002/gcc.20023; Reeser, et al, J Mo/Diagn, 19(5):682-696, 2017. doi: 10.1016/j.jmoldx.2017.05.006; Basturk, et al, Mod Patho/, 30(12):1760-1772, 2017. doi: 10.1038/modpathol.2017.60; Wang, et al, Cancer 123(20):3916-3924, 2017. doi: 10.1002/cncr.30837; Kim, et al, Oncotarget, 8(9):15014-15022, 2017. doi: 10.18632/oncotarget.14788; Busse, et al, Genes Chromosomes Cancer, 56(10):730-749, 2017. doi: 10.1002/gcc.22477; Shi, et al, J Transl Med., 14(1):339, 2016. doi: 10.1186/s12967-016-1075-6, each of which is incorporated by reference herein. FGFR point mutations are known in the art. See, e.g., UniParc entry UPI00000534B8; UniParc entry UPI0000001COF; UniParc entry UPI000002A99A; UniParc entry UPI000012A72A; UniParc entry UPI000059D1C₂; UniParc entry UPI000002A9AC; Uniparc entry UPI000012A72C; Uniparc entry UPI000012A72D; Uniparc entry UPI000013EOB8; Uniparc entry UPI0001CE06A3; Gen bank entry BAD92868.1; Ang et al., Diagn. Mo. Patho. Feb. 24, 2014; U.S. Patent Application Publication No. 2011/0008347; Gallo et al., Cytokine Growth Factor Rev. 26:425-449, 2015; Davies et al., J. Cancer Res. 65:7591, 2005; Kelleher et al., Carcinogenesis 34:2198, 2013; Cazier et al., Nat. Commun. 5:3756, 2014; Liu et al., Genet. Mo. Res. 13:1109, 2014; Trudel et al., Blood 107:4039, 2006; Gallo et al., Cytokine Growth Factor Rev. 26:425, 2015; Liao et al., Cancer Res. 73:5195-5205, 2013; Martincorena et al., Science 348:880 (2015); U.S. Patent Application Publication No. US2016/0235744A1; U.S. Pat. No. 9,254,288B2; U.S. Pat. No. 9,267,176B2; U.S. Patent Application Publication No. 52016/0215350A1; European Patent Application Publication No. EP3023101A1; PCT Patent Application Publication No. WO2016105503A1; Rivera et al., Acta. Neuropatho/., 131(6):847-63, 2016; Lo Iacono et al., Oncotarget., 7(12):14394-404, 2016; Deeken et al., Journal of Clinical Oncology, 34:Supp. Supplement 15, pp. iii93. Abstract Number: el 7520, 2016 Annual Meeting of the American Society of Clinical Oncology, Chicago, IL; Sullivan et al., Journal of Clinical Oncology, 34:Supp. Supplement 15, pp. iii93. Abstract Number: 11596, 2016 Annual Meeting of the American Society of Clinical Oncology, Chicago, IL; Nguyen et al., Molecular Cancer Therapeutics, Vol. 14, No. 12, Supp. 2, Abstract Number: C199, AACR-NCI-EORTC International Conference: Molecular Targets and Cancer Therapeutics, 2015; Li et al., Hum. Patho/., 55:143-50, 2016; European Patent No. EP2203449B1; Yoza et al., Genes Cells., (10):1049-1058, 2016; U.S. Pat. No. 9,254,288B2; European Patent Application Publication No. 3023101A1; PCT Application Publication No. WO 2015/099127A1; European Patent No. EP2203449B1; Yoza et al., Genes Cells., (10):1049-1058, 2016; Bunney et al., EbioMedicine, 2(3):194-204, 2015; Byron et al., Neop/asia, 15(8):975-88, 2013; European Patent Application Publication No. EP3023101A1; PCT Application Publication No. WO 2015/099127A1; Thussbas et al., J. Clin. Oneal., 24(23):3747-55, 2006; Chell et al., Oncogene, 32(25):3059-70, 2013; Tanizaki et al, Cancer Res. 75(15):3149-3146 doi: 10.1158/0008-5472.CAN-14-3771; Yang et al, EBioMedicine pii S2352-3964(18)30218-4. doi: 10.1016/j.ebiom.2018.06.011; Jakobsen, et al Oncotarget 9(40):26195-26208, 2018. doi: 10.18632/oncotarget.25490; Stone, et al Acta Neuropatho 135(1):115-129, 2017. doi: 10.1007/s00401-017-1773-z; Pekmezci et al, Acta Nurotaphol. Commun. 6(1):47. doi: 10.1186/s40478-018-0551-z; De Mattos-Arruda et al, Oncotarget 9(29):20617-20630, 2018. doi:10.18632/oncotarget.25041; Oliveira et al, J Exp Clin Cancer Res 37(1):84, 2018. doi: 10.1186/s13046-018-0746-y; Cha et al, Mo/Oneal 12(7):993-1003, 2018. doi: 10.1002/1878-0261.12194; Ikeda et al, Oncologist, 23(5):586-593, 2018. doi: 10.1634/theoncologist.2017-0479; Pelaez-Garda et al, PLoS One, 8(5):e63695, 2013. doi: 10.1371/journal.pone.0063695; Shimada et al, Oncotarget, 8(55):93567-93579, 2017. doi: 10.18632/oncotarget.20510; Welander et al, World J Surg, 42(2):482-489, 2018. doi: 10.1007/s00268-017-4320-0; Chandrani et al, Ann Oneal, 28(3):597-603, 2017. doi: 10.1093/annonc/mdw636; Dalin et al, Nat Commun, 8(1):1197, 2017. doi: 10.1038/s41467-017-01178-z; Taurin et al, Intl Gyneco Cancer, 28(1):152-160, 2018. doi: 10.1097/IGC.0000000000001129; Haugh et al, J Invest Dermatol 138(2):384-393, 2018. doi: 10.1016/j.jid.2017.08.022; Babina and Turner, Nat Rev Cancer 17(5):318-332, 2017. doi: 10.1038/nrc.2017.8; Greenman et al, Nature 446(7132):153-158, 2007. doi: 10.1038/nature05610; Helsten et al, Clin Cancer Res, 22(1):259-267, 2016. doi: 10.1158/1078-0432.CCR-14-3212; Kim et al, BMC Urol, 18:68, 2018. doi: 10.1186/s12894-018-0380-1; Goyal et al, Cancer Discov, 7(3):252-263, 2017. doi: 10.1158/2159-8290.CD-16-1000; Premov et al, Oncogene, 36(22):3168-3177, 2017. doi: 10.1038/onc.2016.464; Geelvink et al, Int J Mo/Sci. 19(9): pii:E2548, 2018. doi: 10.3390/ijms19092548; Lee et al, Exp Ther Med. 16(2):1343-1349, 2018. doi: 10.3892/etm.2018.6323; Kas et al, Cancer Res, 78(19):5668-5679, 2018. doi: 10.1158/0008-5472.CAN-18-0757; Chesi et al, Blood, 97(3):729-736, 2001. PMID: 11157491. Note that the deletion of FGFR3 isoform Ie residues 795-808 also deletes the stop codon, elongating the protein by 99 amino acids (ATGPQQCEGSLAAHPAAGAQPLPGMRLSADGETATQSFGLCVCVCVCVCVCTSACACVR AHLASRCRGTLGVPAA VQRSPDWCCSTEGPLFWGDPVQNVSGPTRWDPVGQGAGPDMARPLPLHHGTSQGALG PSHTQS); Ge, et al, Am J Cancer Res. 7(7):1540-1553, 2017. PMID: 28744403; Jiao et al, Nat Genet, 45(12):1470-1473, 2013. doi: 10.1038/ng.2813; Jusakul et al, Cancer Discov. 7(10):1116-1135, 2017. doi: 10.1158/2159-8290.CD-17-0368; Guyard et al, Respir Res., 18(1):120, 2018. doi: 10.1186/s12931-017-0605-y; Paik et al, Clin Cancer Res., 23(18):5366-5373, 2017. doi: 10.1158/1078-0432.CCR-17-0645; Roy et al, Mod Patho/., 30(8):1133-1143, 2017. doi: 10.1038/modpathol.2017.33; Chakrabarty et al, Br J Cancer, 117(1):136-143, 2017. doi: 10.1038/bjc.2017.148; Hoang et al, Sci Transl Med., 5(197):197ra102. doi: 10.1126/scitranslmed.3006200; Kim et al, Ann Oneal., 28(6):1250-1259. doi: 10.1093/annonc/mdx098, each of which is incorporated by reference herein.

Compounds of the disclosure have been found to inhibit FGFR1, FGFR2, FGFR3, and/or FGFR4 and are therefore believed to be useful for treating diseases and disorders which can be treated with an inhibitor of FGFR1, FGFR2, FGFR3 and/or FGFR4. For example, compounds of the disclosure can be useful in treating FGFR-associated diseases and disorders, e.g., proliferative disorders such as cancers, including hematological cancers and solid tumor, and angiogenesis-related disorders. Compounds of the disclosure may also be useful in treating disorders arising from autosomal dominant mutations in FGFR, e.g., FGFR3, including, for example, developmental disorders. Developmental disorders to be treated with compounds of the disclosure include Achondroplasia (Ach) and related chondrodysplasia syndromes, including Hypochondroplasia (Hch), Severe Achondroplasia with Developmental Delay and Acanthosis Nigricans (SADDAN), and Thanatophoric dysplasia (TD).

Non-limiting examples of FGFR-associated diseases and disorders include Acanthosis nigricans, Achondroplasia, Apert syndrome, Beare-Stevenson syndrome (BSS), Camptodactyly, tall stature, and hearing loss syndrome (CATSHL) syndrome, cleft lip and palate, congenital heart disease (e.g., associated with ambiguous genitalia), craniosynostosis, Crouzon syndrome, ectrodactyly, encephalocraniocutaneous lipomatosis, Hartsfield syndrome, hypochondroplasia, hypogonadoropic hypogonadism (e.g., hypogonadotropic hypogonadism 2 with or without anosmia, Kallman syndrome), ichthyosis vulgaris and/or atopic dermatitis, Jackson-Weiss syndrome, lethal pulmonary acinar dysplasia, microphthalmia, Muenke coronal craniosynostosis, osteoglophonic dysplasia, Pfeiffer syndrome, seborrheic keratosis, syndactyly, thanatophoric dysplasia (e.g., type I or type II), trigonocephaly 1 (also called metopic craniosynostosis), and tumor-induced osteomalacia.

Non-limiting examples of FGFR1 associated diseases and disorders include congenital heart disease (e.g., associated with ambiguous genitalia), craniosynostosis, encephalocraniocutaneous lipomatosis, Hartsfield syndrome, hypogonadoropic hypogonadism (e.g., hypogonadotropic hypogonadism 2 with or without anosmia, Kallman syndrome), ichthyosis vulgaris and/or atopic dermatitis, Jackson-Weiss syndrome, osteoglophonic dysplasia, Pfeiffer syndrome, trigonocephaly 1 (also called metopic craniosynostosis), and tumor-induced osteomalacia.

Non-limiting examples of FGFR2-associated diseases and disorders include Apert syndrome, Beare-Stevenson syndrome (BSS), Crouzon syndrome, ectrodactyly, Jackson-Weiss syndrome, lethal pulmonary acinar dysplasia, Pfeiffer syndrome, and syndactyly. Non-limiting examples of FGFR3-associated diseases and disorders include acanthosis nigricans, achondroplasia, Camptodactyly, tall stature, and hearing loss syndrome (CATSHL) syndrome, cleft lip and palate, craniosynostosis, hypochondroplasia, microphthalmia, Muenke coronal craniosynostosis, seborrheic keratosis, and thanatophoric dysplasia (e.g., type I or type II). See also, See UniParc entry UPI00000534B8; UniParc entry UPI0000001COF; Uni Pare entry UPI000002A99A; UniParc entry UPI000012A72A;Yong-Xing et al., Hum. Mol. Genet. 9(13):2001-2008, 2000; Eeva-Maria Laitinen et al., PLoS One 7(6):e39450, 2012; Hart et al., Oncogene 19(29):3309-3320, 2000; Shiang et al., Cell 76:335-342, 1994; Rosseau et al., Nature 371:252-254, 1994; Tavormina et al., Nature Genet. 9:321-328, 1995; Bellus et al., Nature Genet. 10:357-359, 1995; Muenke et al., Nature Genet. 8:269-274, 1994; Rutland et al., Nature Genet. 9:173-176, 1995; Reardon et al., Nature Genet. 8:98-103, 1994; Wilkie et al., Nature Genet. 9:165-172, 1995; Jabs et al., Nature Genet. 8:275-279, 1994; Japanese Patent No. JP05868992B2; Ye et al., Plast. Reconstr. Surg., 137(3):952-61, 2016; U.S. Pat. No. 9,447,098B2; Bellus et al., Am. J. Med. Genet. 85(1):53-65, 1999; PCT Patent Application Publication No. WO2016139227A1; Australian Patent Application Publication No. AU2014362227A1; Chinese Patent No. CN102741256B; Ohishi et al., Am. J. Med. Genet. A., doi: 10.1002/ajmg.a.37992, 2016; Nagahara et al., Clin. Pediatr. Endocrinol., 25(3): 103-106, 2016; Hibberd et al., Am. J. Med. Genet. A., doi: 10.1002/ajmg.a.37862, 2016; Dias et al., Exp. Mol. Pathol., 101(1):116-23, 2016; Lin et al., Mol. Med. Rep., 14(3):1941-6, 2016; Barnett et al., Hum. Mutat., 37(9):955-63, 2016; Krstevska-Konstantinova et al., Med. Arch., 70(2):148-50, 2016; Kuentz et al., Br. J. Dermatol., doi: 10.1111/bjd.14681, 2016; Ron et al., Am. J. Case Rep., 15; 17:254-8, 2016; Fernandes et al., Am. J. Med. Genet. A., 170(6):1532-7, 2016; Lindy et al., Am. J. Med. Genet. A., 170(6):1573-9, 2016; Bennett et al., Am. J. Hum. Genet., 98(3):579-87, 2016; lchiyama et al., J. Eur. Acad. Dermatol. Venereal., 30(3):442-5, 2016; Zhao et al., Int. J. Clin. Exp. Med., 8(10):19241-9, 2015; Hasegawa et al., Am. J. Med. Genet. A., 170A(5):1370-2, 2016; Legeai-Mallet, Endocr. Dev., 30:98-105, 2016; Takagi, Am. J. Med. Genet. A., 167A(ll):2851-4, 2015; Goncalves, Fertil. Steril., 104(5):1261-7.el, 2015; Miller et al., Journal of Clinical Oncology, 34:Supp. Supplement 15, pp. iii93. AbstractNumber: e22500, 2016 Annual Meeting of the American Society of Clinical Oncology, Chicago, IL; Sarabipour et al., J. Mol. Biol., 428(20):3903-3910, 2016; Escobar et al., Am. J. Med. Genet. A., 170(7):1908-11, 2016; Mazen et al., Sex Dev., 10(1):16-22, 2016; Taylan et al., J Allergy Clin Immunol, 136(2):507-9, 2015. doi: 10.1016/j.jaci.2015.02.010; Kant et al, EuroJourn Endocrinol, 172(6):763-770, 2015. doi: 10.1530/EJE-14-0945; Gonzalez-Del Angel et al, Am J med Genet A, 176(1):161-166, 2018. doi: 10.1002/ajmg.a.38526; Lei and Deng, Int J Biol Sci 13(9):1163:1171, 2017. doi: 10.7150/ijbs.20792; Lajeunie et al, Eur J Hum Genet, 14(3):289-298, 2006. doi: 10.1038/sj.ejhg.5201558; Karadimas et al, Prenat Diagn, 26(3):258-261, 2006. doi: 10.1002/pd.1392; lbrahimi et al, Hum Mo/Genet 13(19):2313-2324, 2004. doi: 10.1093/hmg/ddh235; Trarbach et al, J Clin Endocrinol Metab., 91(10):4006-4012, 2006. doi: 10.1210/jc.2005-2793; Dode et al, Nat Genet, 33(4):463-465, 2003. doi: 10.1038/ng1122, each of which is incorporated by reference herein.

The term “angiogenesis-related disorder” means a disease characterized in part by an increased number or size of blood vessels in a tissue in a subject or patient, as compared to a similar tissue from a subject not having the disease. Non-limiting examples of angiogenesis-related disorders include: cancer (e.g., any of the exemplary cancers described herein, such as prostate cancer, lung cancer, breast cancer, bladder cancer, renal cancer, colon cancer, gastric cancer, pancreatic cancer, ovarian cancer, melanoma, hepatoma, sarcoma, and lymphoma), exudative macular degeneration, proliferative diabetic retinopathy, ischemic retinopathy, retinopathy of prematurity, neovascular glaucoma, iritis rubeosis, corneal neovascularization, cyclitis, sickle cell retinopathy, and pterygium.

Compounds of the disclosure inhibit wild-type FGFR1, FGFR2, FGFR3, and/or FGFR4. In other aspects, compounds of the disclosure inhibit a mutated FGFR1, FGFR2, FGFR3, and/or FGFR4. In other aspects, compounds of the disclosure inhibit FGFR1, FGFR2, FGFR3, and/or FGFR4 that includes an FGFR kinase inhibitor resistance mutation.

In some embodiments of any of the methods or uses described herein, the cancer (e.g., FGFR-associated cancer) is a hematological cancer. In some embodiments of any of the methods or uses described herein, the cancer (e.g., FGFR-associated cancer) is a solid tumor.

In some embodiments of any of the methods or uses described herein, the cancer (e.g., FGFR-associated cancer) is a lung cancer (e.g., small cell lung carcinoma, non-small cell lung carcinoma, squamous cell carcinoma, lung adenocarcinoma, large cell carcinoma, mesothelioma, lung neuroendocrine carcinoma, smoking-associated lung cancer), prostate cancer, colorectal cancer (e.g., rectal adenocarcinoma), endometrial cancer (e.g., endometrioid endometrial cancer, endometrial adenocarcinoma), breast cancer (e.g., hormone-receptor-positive breast cancer, triple-negative breast cancer, neuroendodrine carcinoma of the breast), skin cancer (e.g., melanoma, cutaneous squamous cell carcinoma, basal cell carcinoma, large squamous cell carcinoma), gallbladder cancer, liposarcoma (e.g., dedifferentiated liposarcoma, myxoid liposarcoma), pheochromocytoma, myoepithelial carcinoma, urothelial carcinoma, spermatocytic seminoma, stomach cancer, head and neck cancer (e.g., head and neck (squamous) carcinoma, head and neck adenoid cystic adenocarcinoma), brain cancer (e.g., glialneural tumors, glioma, neuroblastoma, glioblastoma, pilocytic astrocytoma, Rosette forming glioneural tumor, dysembryoplastic neuroepithelial tumor, anaplastic astrocytoma, medulloblastoma, ganglioglioma, oligodendroglioma), malignant peripheral nerve sheath tumor, sarcoma (e.g., soft tissue sarcoma (e.g., leiomyosarcoma), osteosarcoma), esophageal cancer (e.g., esophageal adenocarcinoma), lymphoma, bladder cancer (e.g., bladder urothelial (transition cell) carcinoma), cervical cancer (e.g., cervical squamous cell carcinoma, cervical adenocarcinoma), fallopian tube cancer (e.g., fallopian tube carcinoma), ovarian cancer (e.g., ovarian serous cancer, ovarian mucinous carcinoma), cholangiocarcinoma, adenoid cystic carcinoma, pancreatic cancer (e.g., pancreatic exocrine carcinoma, pancreatic ductal adenocarcinoma, pancreatic cancer intraepithelial neoplasia), salivary gland cancer (e.g., pleomorphic salivary gland adenocarcinoma, salivary adenoid cystic cancer), oral cancer (e.g., oral squamous cell carcinoma), uterine cancer, gastric or stomach cancer (e.g., gastric adenocarcinoma), gastrointestinal stromal tumors, myeloma (e.g., multiple myeloma), lymphoepithelioma, anal cancer (e.g., anal squamous cell carcinoma), prostate cancer (e.g., prostate adenocarcinoma), renal cell carcinoma, thymic cancer, gastroesophogeal junction adenocarcinoma, testicular cancer, rhabdomyosarcoma (e.g., alveolar rhabdomyosarcoma, embryonic rhabomyosarcoma), renal papillary carcinoma, liver cancer (e.g., hepatocellular carcinoma, intrahepatic cholangiocarcinoma), carcinoid, myeloid proliferative disorders (also called myeloid proliferative neoplasms (MPN); e.g., 8pll myeloproliferative syndrome (EMS, also called stem cell leukemia/lymphoma), acute myeloid leukemia (AML), chronic myeloid leukemia (CML)), lymphoma (e.g., T-cell lymphoma, T-lymphoblastic lymphoma, acute lymphoblastic leukemia (ALL), B-cell lymphoma), myeloid and lymphoid neoplasms, chronic neutrophilic leukemia, phosphaturic mesenchymal tumor, thyroid cancer (e.g. anaplastic thyroid carcinoma), or biliary duct cancer.

In some embodiments of any of the methods or uses described herein, the cancer (e.g., FGFR-associated cancer) is selected from the group of: acute lymphoblastic leukemia (ALL), acute myeloid leukemia (AML), cancer in adolescents, adrenocortical carcinoma, anal cancer, appendix cancer, astrocytoma, atypical teratoid/rhabdoid tumor, basal cell carcinoma, bile duct cancer, bladder cancer, bone cancer, brain stem glioma, brain tumor, breast cancer, bronchial tumor, Burkitt lymphoma, carcinoid tumor, unknown primary carcinoma, cardiac tumors, cervical cancer, childhood cancers, chordoma, chronic lymphocytic leukemia (CLL), chronic myelogenous leukemia (CML), chronic myeloproliferative neoplasms, neoplasms by site, neoplasms, colon cancer, colorectal cancer, craniopharyngioma, cutaneous T-cell lymphoma, cutaneous angiosarcoma, bile duct cancer, ductal carcinoma in situ, embryonal tumors, endometrial cancer, ependymoma, esophageal cancer, esthesioneuroblastoma, Ewing sarcoma, extracranial germ cell tumor, extragonadal germ cell tumor, extrahepatic bile duct cancer, eye cancer, fallopian tube cancer, fibrous histiocytoma of bone, gallbladder cancer, gastric cancer, gastrointestinal carcinoid tumor, gastrointestinal stromal tumors (GIST), germ cell tumor, gestational trophoblastic disease, glioma, hairy cell tumor, hairy cell leukemia, head and neck cancer, thoracic neoplasms, head and neck neoplasms, CNS tumor, primary CNS tumor, heart cancer, hepatocellular cancer, histiocytosis, Hodgkin's lymphoma, hypopharyngeal cancer, intraocular melanoma, islet cell tumors, pancreatic neuroendocrine tumors, Kaposi sarcoma, kidney cancer, Langerhans cell histiocytosis, laryngeal cancer, leukemia, lip and oral cavity cancer, liver cancer, lung cancer, lymphoma, macroglobulinemia, malignant fibrous histiocytoma of bone, osteocarcinoma, melanoma, Merkel cell carcinoma, mesothelioma, metastatic squamous neck cancer, midline tract carcinoma, mouth cancer, multiple endocrine neoplasia syndromes, multiple myeloma, mycosis fungoides, myelodysplastic syndromes, myelodysplastic/myeloproliferative neoplasms, neoplasms by site, neoplasms, myelogenous leukemia, myeloid leukemia, multiple myeloma, myeloproliferative neoplasms, nasal cavity and para nasal sinus cancer, nasopharyngeal cancer, neuroblastoma, non-Hodgkin's lymphoma, non-small cell lung cancer, lung neoplasm, pulmonary cancer, pulmonary neoplasms, respiratory tract neoplasms, bronchogenic carcinoma, bronchial neoplasms, oral cancer, oral cavity cancer, lip cancer, oropharyngeal cancer, osteosarcoma, ovarian cancer, pancreatic cancer, papillomatosis, paraganglioma, para nasal sinus and nasal cavity cancer, parathyroid cancer, penile cancer, pharyngeal cancer, pheochromosytoma, pituitary cancer, plasma cell neoplasm, pleuropulmonary blastoma, pregnancy-associated breast cancer, primary central nervous system lymphoma, primary peritoneal cancer, prostate cancer, rectal cancer, colon cancer, colonic neoplasms, renal cell cancer, retinoblastoma, rhabdomyosarcoma, salivary gland cancer, sarcoma, Sezary syndrome, skin cancer, Spitz tumors, small cell lung cancer, small intestine cancer, soft tissue sarcoma, squamous cell carcinoma, squamous neck cancer, stomach cancer, T-cell lymphoma, testicular cancer, throat cancer, thymoma and thymic carcinoma, thyroid cancer, transitional cell cancer of the renal pelvis and ureter, unknown primary carcinoma, urethral cancer, uterine cancer, uterine sarcoma, vaginal cancer, vulvar cancer, and Wilms' tumor.

In some embodiments, a hematological cancer (e.g., hematological cancers that are FGFR associated cancers) is selected from the group consisting of leukemias, lymphomas (non-Hodgkin's lymphoma), Hodgkin's disease (also called Hodgkin's lymphoma), and myeloma, for instance, acute lymphocytic leukemia (ALL), acute myeloid leukemia (AML), acute promyelocytic leukemia (APL), chronic lymphocytic leukemia (CLL), chronic myeloid leukemia (CMIL), chronic myelomonocytic leukemia (CMML), chronic neutrophilic leukemia (CNL), acute undifferentiated leukemia (AUL), anaplastic large-cell lymphoma (ALCL), prolymphocytic leukemia (PML), juvenile myelomonocyctic leukemia (JMML), adult Tcell ALL, AML with trilineage myelodysplasia (AML/TMDS), mixed lineage leukemia (MLL), myelodysplastic syndromes (MDSs), myeloproliferative disorders (MPD), and multiple myeloma (MM).

Additional examples of hematological cancers include myeloproliferative disorders (MPD) such as polycythemia vera (PV), essential thrombocytopenia (ET) and idiopathic primary myelofibrosis (IMF/IPF/PMF). In some embodiments, the hematological cancer (e.g., the hematological cancer that is a FGFR-associated cancer) is AML or CMML. In some embodiments, the cancer (e.g., the FGFR-associated cancer) is a solid tumor. Examples of solid tumors (e.g., solid tumors that are FGFR-associated cancers) include, for example, lung cancer (e.g., lung adenocarcinoma, non-small-cell lung carcinoma, squamous cell lung cancer), bladder cancer, colorectal cancer, brain cancer, testicular cancer, bile duct cancer cervical cancer, prostate cancer, and sparmatocytic seminomas. See, for example, Turner and Grose, Nat. Rev. Cancer, 10(2):116-129, 2010.

In some embodiments, the cancer is selected from the group consisting of bladder cancer, brain cancer, breast cancer, cholangiocarcinoma, head and neck cancer, lung cancer, multiple myeloma, rhabdomyosarcoma, urethral cancer, uterine cancer. In some embodiments, the cancer is selected from the group consisting of lung cancer, breast cancer, and brain cancer.

In some embodiments, the cancer is hepatocellular carcinoma.

In some embodiments, a FGFR1-associated cancer is selected from the group consisting of lung cancer, breast cancer, and brain cancer.

In some embodiments, the cancer is selected from the group consisting of breast cancer, uterine cancer, cholangiocarcinoma, and lung cancer.

In some embodiments, a FGFR2-associated cancer is selected from the group consisting of breast cancer, uterine cancer, cholangiocarcinoma, and lung cancer. In some embodiments, the cancer is selected from the group consisting of lung cancer, bladder cancer, urethral cancer, multiple myeloma, and head and neck cancer.

In some embodiments, a FGFR3-associated cancer is selected from the group consisting of lung cancer, bladder cancer, urethral cancer, multiple myeloma, and head and neck cancer.

In some embodiments, the cancer is selected from lung cancer, rhabdomyosarcoma, and breast cancer.

In some embodiments, a FGFR4-associated cancer is selected from hepatocellular carcinoma, lung cancer, rhabdomyosarcoma, and breast cancer.

In some aspects, the compounds of the disclosure are useful in treating cancers associated with amplification or overexpression of FGFR1, for example, Breast cancer or carcinoma (e.g., hormone receptor-positive breast cancer, ductal carcinoma in situ (breast)), pancreatic ductal adenocarcinoma, pancreatic exocrine carcinoma, smoking-associated lung cancer, small cell lung cancer, lung adenocarcinoma, non-small cell lung cancer, squamous cell lung cancer or carcinoma, prostate cancer or carcinoma, ovarian cancer, fallopian tube carcinoma, bladder cancer, rhabdomyosarcoma, head and neck carcinoma (e.g., head and neck squamous cell carcinoma), esophageal cancer (e.g., esophageal squamous cell carcinoma), sarcoma (e.g., osteosarcoma), hepatocellular carcinoma, renal cell carcinoma, colorectal cancer (e.g., colorectal adenocarcinoma), prostate cancer, salivary gland tumors, glioblastoma multiforme, urinary bladder cancer, urothelial carcinoma, carcinoma of unknown primary, squamous non-lung tumors, gastric cancer, gastroesophageal junction carcinoma, adenoid cystic carcinoma, anal squamous cell carcinoma, oral squamous cell carcinoma, cholangiocarcinoma, hemangioendothelioma, leiomyosarcoma, melanoma, neuroendocrine carcinoma, squamous cell carcinoma, uterine carcinosarcoma.

In some aspects, the compounds of the disclosure are useful in treating cancers associated with amplification of FGFR2, for example, Gastric cancer, gastroesophageal junction adenocarcinoma, breast cancer (e.g., triple negative breast cancer), colon cancer, colorectal cancer (e.g., colorectal adenocarcinoma), urothelial cancer, bladder adenocarcinoma, carcinoma of unknown primary, cholangiocarcinoma, endometrial adenocarcinoma, esophageal adenocarcinoma, gallbladder carcinoma, ovarian cancer, fallopian tube carcinoma, pancreatic exocrine carcinoma, sarcoma, squamous cell carcinoma.

In some aspects, the compounds of the disclosure are useful in treating cancers associated with overexpression of FGFR2, for example, Myxoid lipocarcinoma, rectal cancer, renal cell carcinoma, breast cancer.

In some aspects, the compounds of the disclosure are useful in treating cancers associated with upregulation of activity of FGFR3, for example, Colorectal cancer, hepatocellular carcinoma, pancreatic exocrine carcinoma. In some aspects, the compounds of the disclosure are useful in treating cancers associated with overexpression of activity of FGFR3, for example, Multiple myeloma, thyroid carcinoma. In some aspects, the compounds of the disclosure are useful in treating cancers associated with amplification of activity of FGFR3, for example, Bladder cancer and salivary adenoid cystic cancer, urothelial cancer, breast cancer, carcinoid, carcinoma of unknown primary, colorectal cancer (e.g., colorectal adenocarcinoma), gallbladder carcinoma, gastric cancer, gastroesophageal junction adenocarcinoma, glioma, mesothelioma, non-small cell lung carcinoma, small cell lung cancer, ovarian cancer, fallopian tube carcinoma, pancreatic exocrine carcinoma.

In some aspects, the compounds of the disclosure are useful in treating cancers associated with amplification of FGFR4, for example, Rhabdomyosarcoma, prostate cancer or carcinoma, breast cancer, urothelial cancer, carcinoid, carcinoma of unknown primary, esophageal adenocarcinoma, head and neck carcinoma, hepatocellular carcinoma, non-small cell lung carcinoma, ovarian cancer, fallopian tube carcinoma, peritoneal carcinoma, renal cell carcinoma.

In some aspects, the compounds of the disclosure are useful in treating cancers associated with upregulation of activity of FGFR4, for example, Colorectal cancer, hepatocellular carcinoma, adrenal carcinoma, breast cancer.

In some aspects, the compounds of the disclosure are useful in treating cancers associated with overexpression of activity of FGFR4, for example, Pancreatic intraepithelial neoplasia, and pancreatic ductal adenocarcinoma.

In some aspects, the compounds of the disclosure are more selective for one FGFR than for another. As used herein, the “selectivity” of a compound for a first target over a second target means that the compound has more potent activity at the first target than the second target. A fold selectivity can be calculated by any method known in the art. For example, a fold selectivity can be calculated by dividing the IC50 value (or Kd value) of a compound for the second target (e.g., FGFR1) by the IC50 value (or Kd value) of the same compound for the first target (e.g., FGFR2 or FGFR3). An IC50 value (or Kd value) can be determined by any method known in the art. In some embodiments, a compound is first determined to have an activity of less than 500 nM for the first target. In some embodiments, a compound is first determined to have an activity of less than 500 nM for the second target.

For example, in some aspects, the compounds of the disclosure are more selective for FGFR3 than for FGFR1. In some aspects, the compounds are at least 3-fold more selective for FGFR3 than for FGFR1. In some aspects, the compounds are 4, 5, 6, 7, 8, 9, 10, 15, 20, 30, 40, 50, 75, 100, 200, 500, or 1000 fold more selective for FGFR3 than for FGFR1.

For example, in some aspects, the compounds of the disclosure are more selective for FGFR4 than for FGFR1. In some aspects, the compounds are at least 3-fold more selective for FGFR4 than for FGFR1. In some aspects, the compounds are 4, 5, 6, 7, 8, 9, 10, 15, 20, 30, 40, 50, 75, 100, 200, 500, or 1000 fold more selective for FGFR4 than for FGFR1.

In some aspects, the compounds of the disclosure are more selective for FGFR2 than for FGFR1. In some aspects, the compounds are at least 3-fold more selective for FGFR2 than for FGFR1. In some aspects, the compounds are 4, 5, 6, 7, 8, 9, 10, 15, 20, 30, 40, 50, 75, 100, 200, 500, or 1000 fold more selective for FGFR2 than for FGFR1.

In some aspects, the compounds of the disclosure are more selective for a first FGFR family member (e.g., FGFR2 or FGFR3) over a second FGFR family member (e.g., FGFR1 or FGFR4). In some aspects, the compounds of the disclosure are at least 3-fold more selective for a first FGFR family member over a second FGFR family member. In some aspects, the compounds are at least 10, 20, 30, 40, 50, 60, 70, 80, 90, 200, 300, 400, 500, 600, 700, 800, 900, or at least 1000 fold more selective for a first FGFR family member over a second FGFR family member.

In some aspects, the compounds of the disclosure are more selective for a first FGFR family member (e.g., FGFR4 or FGFR3) over a second FGFR family member (e.g., FGFR1 or FGFR2). In some aspects, the compounds of the disclosure are at least 3-fold more selective for a first FGFR family member over a second FGFR family member. In some aspects, the compounds are at least 10, 20, 30, 40, 50, 60, 70, 80, 90, 200, 300, 400, 500, 600, 700, 800, 900, or at least 1000 fold more selective for a first FGFR family member over a second FGFR family member.

In some aspects, the compounds of the disclosure are more selective for an FGFR kinase over another kinase that is not an FGFR kinase. For example, the compounds of the disclosure are at least 3-fold more selective for an FGFR kinase over another kinase that is not an FGFR kinase. In some aspects, the compounds of the disclosure are at least 10, 20, 30, 40, 50, 60, 70, 80, 90, 200, 300, 400, 500, 600, 700, 800, 900, or at least 1000 fold more selective for an FGFR kinase over another kinase that is not an FGFR kinase. Kinases that are not FGFR kinases include, for example, KDR kinase and Aurora B kinase.

In some embodiments, the compounds of the disclosure exhibit brain and/or central nervous system (CNS) penetrance. Such compounds are capable of crossing the blood brain barrier and inhibiting a FGFR kinase in the brain and/or other CNS structures. In some embodiments, the compounds provided herein are capable of crossing the blood brain barrier in a therapeutically effective amount. For example, treatment of a subject with cancer (e.g., a FGFR-associated cancer such as a FGFR-associated brain or CNS cancer) can include administration (e.g., oral administration) of the compound to the subject. In some such embodiments, the compounds provided herein are useful for treating a primary brain tumor or metastatic brain tumor. For example, a FGFR-associated primary brain tumor or metastatic brain tumor.

In some embodiments, the compounds of the disclosure, exhibit one or more of high GI absorption, low clearance, and low potential for drug-drug interactions.

In some aspects, compounds of the disclosure can be used for treating a subject diagnosed with (or identified as having) a FGFR-associated disease or disorder (e.g., a FGFR-associated cancer) that include administering to the subject a therapeutically effective amount of a compound of the disclosure. Also provided herein are methods for treating a subject identified or diagnosed as having a FGFR-associated disease or disorder (e.g., a FGFR-associated cancer) that include administering to the subject a therapeutically effective amount of a compound of the disclosure. In some embodiments, the subject that has been identified or diagnosed as having a FGFR-associated disease or disorder (e.g., a FGFR-associated cancer) through the use of a regulatory agency-approved, e.g., FDA-approved test or assay for identifying dysregulation of a FGFR gene, a FGFR kinase, or expression or activity or level of any of the same, in a subject or a biopsy sample from the subject or by performing any of the non-limiting examples of assays described herein. In some embodiments, the test or assay is provided as a kit. In some embodiments, the FGFR-associated disease or disorder is a FGFR-associated cancer. For example, the FGFR-associated cancer can be a cancer that includes one or more FGFR inhibitor resistance mutations.

Also provided are methods for treating a disease or disorder in a subject in need thereof, the method comprising: (a) detecting a FGFR-associated disease or disorder in the subject; and (b) administering to the subject a therapeutically effective amount of a compound of the disclosure. Some embodiments of these methods further include administering to the subject an additional therapy or therapeutic agent (e.g., a second FGFR inhibitor, a second compound of the disclosure, or an immunotherapy. In some embodiments, the subject was previously treated with a first FGFR inhibitor or previously treated with another treatment. In some embodiments, the subject is determined to have a FGFR-associated disease or disorder through the use of a regulatory agency-approved, e.g., FDA approved test or assay for identifying dysregulation of a FGFR gene, a FGFR kinase, or expression or activity or level of any of the same, in a subject or a biopsy sample from the subject or by performing any of the non-limiting examples of assays described herein. In some embodiments, the test or assay is provided as a kit.

Also provided are methods for treating cancer in a subject in need thereof, the method comprising: (a) detecting a FGFR-associated cancer in the subject; and (b) administering to the subject a therapeutically effective amount of a compound of the disclosure. Some embodiments of these methods further include administering to the subject an additional therapy or therapeutic agent (e.g., a second FGFR inhibitor, a second compound of the disclosure, or an immunotherapy). In some embodiments, the subject was previously treated with a first FGFR inhibitor or previously treated with another anticancer treatment, e.g., at least partial resection of the tumor or radiation therapy. In some embodiments, the subject is determined to have a FGFR-associated cancer through the use of a regulatory agency-approved, e.g., FDA-approved test or assay for identifying dysregulation of a FGFR gene, a FGFR kinase, or expression or activity or level of any of the same, in a subject or a biopsy sample from the subject or by performing any of the non-limiting examples of assays described herein. In some embodiments, the test or assay is provided as a kit. In some embodiments, the cancer is a FGFR associated cancer. For example, the FGFR-associated cancer can be a cancer that includes one or more FGFR inhibitor resistance mutations. In some embodiments, the cancer is a FGFR associated cancer. For example, the FGFR-associated cancer can be a cancer that includes one or more FGFR activating mutations.

Also provided are methods of treating a subject that include performing an assay on a sample obtained from the subject to determine whether the subject has a dysregulation of a FGFR gene, a FGFR kinase, or expression or activity or level of any of the same, and administering (e.g., specifically or selectively administering) a therapeutically effective amount of a compound of the disclosure or pharmaceutically acceptable salt or solvate thereof to the subject determined to have a dysregulation of a FGFR gene, a FGFR kinase, or expression or activity or level of any of the same. Some embodiments of these methods further include administering to the subject an additional therapy or therapeutic agent (e.g., a second FGFR inhibitor, a second compound of the disclosure, or immunotherapy). In some embodiments of these methods, the subject was previously treated with a first FGFR inhibitor or previously treated with another anticancer treatment, e.g., at least partial resection of a tumor or radiation therapy. In some embodiments, the subject is a subject suspected of having a FGFR-associated disease or disorder (e.g., a FGFR-associated cancer), a subject presenting with one or more symptoms of a FGFR-associated disease or disorder (e.g., a FGFR-associated cancer), or a subject having an elevated risk of developing a FGFR-associated disease or disorder (e.g., a FGFR-associated cancer). In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. Additional, non-limiting assays that may be used in these methods are described herein. Additional assays are also known in the art. In some embodiments, the dysregulation of a FGFR gene, a FGFR kinase, or expression or activity or level of any of the same includes one or more FGFR inhibitor resistance mutations.

Also provided herein are methods of selecting a treatment for a subject, wherein the methods include a step of performing an assay on a sample obtained from the subject to determine whether the subject has a dysregulation of a FGFR gene, a FGFR kinase, or expression or activity or level of any of the same (e.g., one or more FGFR inhibitor resistance mutations), and identifying or diagnosing a subject determined to have a dysregulation of a FGFR gene, a FGFR kinase, or expression or activity or level of any of the same, as having a FGFR-associated cancer. Some embodiments further include administering the selected treatment to the subject identified or diagnosed as having a FGFR-associated cancer. For example, in some embodiments, the selected treatment can include administration of a therapeutically effective amount of a compound of the disclosure to the subject identified or diagnosed as having a FGFR-associated cancer. In some embodiments, the assay is an in vitro assay. For example, an assay that utilizes the next generation sequencing, immunohistochemistry, or break apart FISH analysis. In some embodiments, the assay is a regulatory agency-approved, e.g., FDA-approved, kit. In some embodiments, the assay is a liquid biopsy.

Also provided herein are methods of treating a FGFR-associated cancer in a subject that include (a) administering one or more (e.g., two or more, three or more, four or more, five or more, or ten or more) doses of a first FGFR kinase inhibitor to a subject identified or diagnosed as having a FGFR associated cancer (e.g., any of the types of FGFR-associated cancers described herein) (e.g., identified or diagnosed as having a FGFR-associated cancer using any of the exemplary methods described herein or known in the art); (b) after step (a), determining a level of circulating tumor DNA in a biological sample (e.g., a biological sample comprising blood, serum, or plasma) obtained from the subject; (c) administering a therapeutically effective amount of a second FGFR inhibitor or a compound of the disclosure as a monotherapy or in conjunction with an additional therapy or therapeutic agent to a subject identified as having about the same or an elevated level of circulating tumor DNA as compared to a reference level of circulating tumor DNA (e.g., any of the reference levels of circulating tumor DNA described herein). In some examples of these methods, the reference level of circulating tumor DNA is a level of circulating tumor DNA in a biological sample obtained from the subject prior to step (a). Some embodiments of these methods further include determining the level of circulating tumor DNA in the biological sample obtained from the subject prior to step (a). In some examples of these methods, the reference level of circulating tumor DNA is a threshold level of circulating tumor DNA (e.g., an average level of circulating tumor DNA in a population of subjects having a similar FGFR-associated cancer and having a similar stage of the FGFR-associated cancer, but receiving a non-effective treatment or a placebo, or not yet receiving therapeutic treatment, or a level of circulating tumor DNA in a subject having a similar FGFR-associated cancer and having a similar stage of the FGFR-associated cancer, but receiving a non-effective treatment or a placebo, or not yet receiving therapeutic treatment). In some examples of these methods, the first FGFR inhibitor is: ARQ-087, ASP5878, AZD4547, B-701, BAY1179470, BAY1187982, BGJ398, brivanib, Debio 1347, dovitinib, E7090, erdafitinib, FPA144, HMPL-453, INCB054828, lenvatinib, lucitanib, LY3076226, MAX-40279, nintedanib, orantinib, pemigatinib, ponatinib, PRN1371, rogaratinib, sulfatinib, roblitinib, ICP-105, BIG-1262, futibatinib, fisogatinib, LOXO-435, or RLY-4008.

Compounds of the disclosure can also be administered with additional therapy or therapeutic agents. In some aspects, the additional therapy or therapeutic agent includes one or more of radiation therapy, a chemotherapeutic agent (e.g., any of the exemplary chemotherapeutic agents described herein or known in the art), a checkpoint inhibitor (e.g., any of the exemplary checkpoint inhibitors described herein or known in the art), surgery (e.g., at least partial resection of the tumor), and one or more other kinase inhibitors (e.g., any of the kinase inhibitors described herein or known in the art).

Compounds of the disclosure may also be useful as adjuvants to cancer treatment, that is, they can be used in combination with one or more additional therapies or therapeutic agents, for example a chemotherapeutic agent that works by the same or by a different mechanism of action. In some embodiments, a compound of the disclosure can be used prior to administration of an additional therapeutic agent or additional therapy. For example, a subject in need thereof can be administered one or more doses of a compound of the disclosure for a period of time and then under go at least partial resection of the tumor. In some embodiments, the treatment with one or more doses of a compound of the disclosure reduces the size of the tumor (e.g., the tumor burden) prior to the at least partial resection of the tumor. In some embodiments, a subject has a cancer (e.g., a locally advanced or metastatic tumor) that is refractory or intolerant to standard therapy (e.g., administration of a chemotherapeutic agent, such as a first FGFR inhibitor or a multikinase inhibitor, immunotherapy, radiation, or a platinum-based agent (e.g., cisplatin)). In some embodiments, a subject has a cancer (e.g., a locally advanced or metastatic tumor) that is refractory or intolerant to prior therapy (e.g., administration of a chemotherapeutic agent, such as a first FGFR inhibitor or a multikinase inhibitor, immunotherapy, radiation, or a platinum-based agent (e.g., cisplatin)).

In some embodiments of any the methods described herein, the compound of the disclosure is administered in combination with a therapeutically effective amount of at least one additional therapeutic agent selected from one or more additional therapies or therapeutic (e.g., chemotherapeutic) agents. Non-limiting examples of additional therapeutic agents include: other FGFR-targeted therapeutic agents (i.e. a first or second FGFR kinase inhibitor), other kinase inhibitors (e.g., receptor tyrosine kinase targeted therapeutic agents (e.g., Trk inhibitors or EGFR inhibitors)), signal transduction pathway inhibitors, checkpoint inhibitors, modulators of the apoptosis pathway (e.g. obataclax); cytotoxic chemotherapeutics, angiogenesis-targeted therapies, immune-targeted agents, including immunotherapy, and radiotherapy.

Also provided herein are methods of treating a disease or disorder, comprising administering to a subject in need thereof a pharmaceutical combination for treating the disease or disorder which comprises (a) a compound of the disclosure, (b) an additional therapeutic agent, and (c) optionally at least one pharmaceutically acceptable carrier for simultaneous, separate or sequential use for the treatment of the disease or disorder, wherein the amounts of the compound of the disclosure and the additional therapeutic agent are together effective in treating the disease or disorder. In some embodiments, the compound of the disclosure, and the additional therapeutic agent are administered simultaneously as separate dosages. In some embodiments, the compound of the disclosure, and the additional therapeutic agent are administered as separate dosages sequentially in any order, in jointly therapeutically effective amounts, e.g. in daily or intermittently dosages. In some embodiments, the compound of the disclosure, and the additional therapeutic agent are administered simultaneously as a combined dosage. In some embodiments, the disease or disorder is a FGFR-associated disease or disorder. In some embodiments, the subject has been administered one or more doses of a compound of of the disclosure, prior to administration of the pharmaceutical composition.

In some embodiments, the treatment period is at least 7 days (e.g., at least or about 8 days, at least or about 9 days, at least or about 10 days, at least or about 11 days, at least or about 12 days, at least or about 13 days, at least or about 14 days, at least or about 15 days, at least or about 16 days, at least or about 17 days, at least or about 18 days, at least or about 19 days, at least or about 20 days, at least or about 21 days, at least or about 22 days, at least or about 23 days, at least or about 24 days, at least or about 25 days, at least or about 26 days, at least or about 27 days, at least or about 28 days, at least or about 29 days, or at least or about 30 days).

In some embodiments, the treatment period is at least 21 days (e.g., at least or about 22 days, at least or about 23 days, at least or about 24 days, at least or about 25 days, at least or about 26 days, at least or about 27 days, at least or about 28 days, at least or about 29 days, at least or about 30 days, at least or about 31 days, at least or about 32 days, at least or about 33 days, at least or about 34 days, at least or about 35 days, at least or about 36 days, at least or about 37 days, at least or about 38 days, at least or about 39 days, or at least or about 40 days).

Also provided herein are pharmaceutical compositions that contain, as the active ingredient, a compound of the disclosure, in combination with one or more pharmaceutically acceptable carriers (excipients). In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is formulated for oral administration. In some embodiments, the composition is formulated as a tablet or capsule.

The compositions comprising a compound of the disclosure can be formulated in a unit dosage form, each dosage containing from about 5 to about 1,000 mg (1 g), more usually about 100 mg to about 500 mg, of the active ingredient. The term “unit dosage form” refers to physically discrete units for human subjects and other subjects, each unit containing a predetermined quantity of active material (i.e., a compound of the disclosure) to produce the desired therapeutic effect, with a suitable pharmaceutical excipient.

In some embodiments, the compositions provided herein contain from about 5 mg to about 50 mg of the active ingredient, i.e., the compound of the disclosure. One having ordinary skill in the art will appreciate that this embodies compounds or compositions containing about 5 mg to about 10 mg, about 10 mg to about 15 mg, about 15 mg to about 20 mg, about 20 mg to about 25 mg, about 25 mg to about 30 mg, about 30 mg to about 35 mg, about 35 mg to about 40 mg, about 40 mg to about 45 mg, or about 45 mg to about 50 mg of the active ingredient. In some embodiments, the compositions provided herein contain from about 50 mg to about 500 mg of the active ingredient. One having ordinary skill in the art will appreciate that this embodies compounds or compositions containing about 50 mg to about 100 mg, about 100 mg to about 150 mg, about 150 mg to about 200 mg, about 200 mg to about 250 mg, about 250 mg to about 300 mg, about 350 mg to about 400 mg, or about 450 mg to about 500 mg of the active ingredient. In some embodiments, the compositions provided herein contain from about 500 mg to about 1,000 mg of the active ingredient. One having ordinary skill in the art will appreciate that this embodies compounds or compositions containing about 500 mg to about 550 mg, about 550 mg to about 600 mg, about 600 mg to about 650 mg, about 650 mg to about 700 mg, about 700 mg to about 750 mg, about 750 mg to about 800 mg, about 800 mg to about 850 mg, about 850 mg to about 900 mg, about 900 mg to about 950 mg, or about 950 mg to about 1,000 mg of the active ingredient.

The active compound may be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual subject, the severity of the subject's symptoms, and the like.

In some embodiments, the compounds provided herein can be administered in an amount ranging from about 1 mg/kg to about 100 mg/kg. In some embodiments, the compound provided herein can be administered in an amount of about 1 mg/kg to about 20 mg/kg, about 5 mg/kg to about 50 mg/kg, about 10 mg/kg to about 40 mg/kg, about 15 mg/kg to about 45 mg/kg, about 20 mg/kg to about 60 mg/kg, or about 40 mg/kg to about 70 mg/kg. For example, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg. In some embodiments, such administration can be once-daily or twice-daily (BID) administration.

The disclosure is also directed to the following aspects:
Aspect 1. A compound of formula (I):

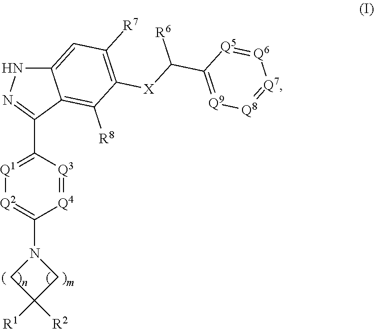

or a pharmaceutically acceptable salt thereof, wherein
   X═O, S, or NR;
      R is H or C₁-C₃alkyl;
   n=1 or 2;
   m=1 or 2;
   R¹ is H or optionally substituted C₁-C₆alkyl;
   R² is H, optionally substituted C₁-C₆alkyl, —NR³R⁴, —OR^{4a}, —P(O)R^{4b}R^{4c}, —SO₂R³, or —C(O)NR³R⁴;
      R³ is H or C₁-C₆alkyl;
      R⁴ is H, optionally substituted C₁-C₆alkyl, optionally substituted C₂-C₆alkenyl, C₃-C₅cycloalkyl, 3- to 6-membered heterocycloalkyl, or C(O)(CH₂)₀₋₃NR^{4d}R^{4e};
      or R³ and R⁴, together with the N atom to which they are both attached, form a 3- to 6-membered heterocycloalkyl optionally substituted with one or more substituents that are each C₁-C₆alkyl, C₁-C₆alkoxyl, F, or OH;
      R^{4a} is H, optionally substituted C₁-C₆alkyl or C₃-C₅cycloalkyl;
      R^{4b} and R^{4c} are each independently C₁-C₆alkyl; or R^{4b} and R^{4c} together with the phosphorus atom to which they are both attached, form a 4- to 6-membered heterocycloalkyl ring;
      R^{4d} and R^{4e} are each independently H or C₁-C₆alkyl, or R^{4d} and R^{4e}, together with the nitrogen atom to which they are both attached, form a 4- to 6-membered heterocycloalkyl ring;
   or R¹ and R², together with the carbon atom to which they are both attached, form a 3-5 membered cycloalkyl ring;
   one or two of Q¹, Q², Q³, Q⁴ is N and the others are each independently CR^{5a};
      R^{5a} is H, halogen, —CN, —S(O)₂C₁-C₆alkyl, OCF₃, OC₁-C₃alkyl, or C₁-C₃alkyl;
   Q⁵, Q⁶, Q⁷, Q⁸, and Q⁹ are each independently N or CR⁵, wherein one or two of the Q⁵, Q⁶, Q⁷, Q⁸, and Q⁹ is N and the remainder are CR⁵;
      R⁵ is H, halogen, C₁-C₃alkyl, C₁-C₃alkoxyl, or cycloalkyl;
   R⁶ is C₁-C₆alkyl;
   R⁷ is H, halogen, —C₁-C₆alkyl, —C₁-C₆ alkoxyl, or -cycloalkyl; and
   R⁸ is H, halogen, —C₁-C₆alkyl, —C₁-C₆ alkoxyl, or -cycloalkyl.
Aspect 2. The compound of aspect 1, wherein R¹ is H or C₁-C₆alkyl; and R⁴ is H, optionally substituted C₁-C₆alkyl, C₃-C₅cycloalkyl, 3- to 6-membered heterocycloalkyl, or C(O)(CH₂)₀₋₃NR^{4d}R^{4e}.
Aspect 3. The compound of aspect 1 or aspect 2, wherein X is O.
Aspect 4. The compound of any one of aspects 1-3, wherein R⁶ is CH₃.
Aspect 5. The compound of any one of the preceding aspects, wherein R⁸ is H.
Aspect 6. The compound of any one of the preceding aspects, wherein R⁷ is H, F, or OCH₃.
Aspect 7. The compound of any one of the preceding aspects, wherein Q¹ and Q³ are each CR^{5a} wherein R^{5a} is H.
Aspect 8. The compound of any one of the preceding aspects, wherein Q² and Q⁴ are each N.
Aspect 9. The compound of any one of aspects 1-7, wherein Q² is N and Q⁴ is CR^{5a} wherein R^{5a} is H or F.
Aspect 10. The compound of any one of aspects 1-7, wherein Q² is N and Q⁴ is CR^{5a} wherein R^{5a} is CN, S(O)₂C₁-C₆alkyl, OC₁-C₃alkyl, or C₁-C₃alkyl.
Aspect 11. The compound of any one of the preceding aspects, wherein Q⁵ and Q⁹ are each CR⁵ wherein each R⁵ is C₁; Q⁷ is N; Q⁶ is CR⁵ wherein R⁵ is H; and Q⁸ is CR⁵ wherein R⁵ is H or CH₃.
Aspect 12. The compound of any one of the preceding aspects, wherein n is 1 and m is 1.
Aspect 13. The compound of any one of the preceding aspects, wherein the compound of formula (I) is a compound of formula (IA):

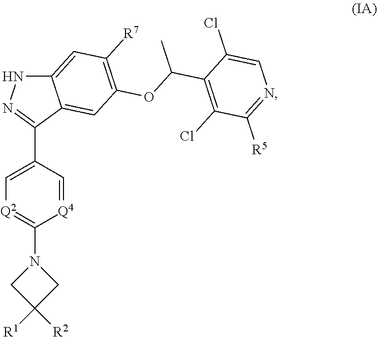

or a pharmaceutically acceptable salt thereof, wherein
Q² and Q⁴ are each N, or one of Q² or Q⁴ is N and the other is CR^{5a};
   R^{5a} is H, F, —SO₂CH₃, or —CN;
R⁵ is H or CH₃; and
R⁷ is H, F, or OCH₃.
Aspect 14. The compound of aspect 13, wherein R⁵ is H.
Aspect 15. The compound of aspect 13, wherein R⁵ is CH₃.
Aspect 16. The compound of any one of aspects 13-15, wherein R⁷ is H.
Aspect 17. The compound of any one of aspects 13-15, wherein R⁷ is F.
Aspect 18. The compound of any one of aspects 13-15, wherein R⁷ is OCH₃.
Aspect 19. The compound of any one of aspects 13-18, wherein Q² and Q⁴ are each N.
Aspect 20. The compound of any one of aspects 13-18, wherein one of Q² or Q⁴ is N and the other is CR^{5a}.
Aspect 21. The compound of aspect 20, wherein R^{5a} is H or F.
Aspect 22. The compound of aspect 21, wherein R^{5a} is H.
Aspect 23. The compound of aspect 21, wherein R^{5a} is F.
Aspect 24. The compound of aspect 20, wherein R^{5a} is CN or SO₂CH₃.
Aspect 25. The compound of aspect 24, wherein R^{5a} is CN.
Aspect 26. The compound of aspect 24, wherein R^{5a} is SO₂CH₃.
Aspect 27. The compound of aspect 1 or aspect 2, wherein R^{5a} is OCF₃.
Aspect 28. The compound of any one of aspects 1-27, wherein R¹ is H.
Aspect 29. The compound of any one of aspects 1-27, wherein R¹ is optionally substituted C₁-C₆alkyl.
Aspect 30. The compound of aspect 29, wherein R¹ is —CH₂CN.
Aspect 31. The compound of aspect 29, wherein R¹ is CH₃.
Aspect 32. The compound of any one of aspects 1-31, wherein R² is H.
Aspect 33. The compound of any one of aspects 1-31, wherein R² is optionally substituted C₁-C₆alkyl.
Aspect 34. The compound of aspect 33, wherein R² is unsubstituted C₁-C₆alkyl, such as CH₃.
Aspect 35. The compound of aspect 33, wherein R² is substituted C₁-C₆alkyl.
Aspect 36. The compound of aspect 35, wherein the C₁-C₆alkyl is substituted with —NHSO₂(C₁-C₆alkyl), —N(C₁-C₆alkyl)SO₂(C₁-C₆alkyl), 5- to 6-membered heterocycloakyl, —NH(C₁-C₆alkyl), —N(C₁-C₆alkyl)₂, or —P(O)R^{4b}R^{4c} wherein R^{4b} and R^{4c} are independently C₁-C₆alkyl; or R^{4b} and R^{4c} together with the phosphorus atom to which they are both attached, form a 4- to 6-membered heterocycloalkyl ring.
Aspect 37. The compound of aspect 36, wherein R² is —CH₂—NHSO₂(CH₃), —CH₂—N(CH₃)SO₂(CH₃),

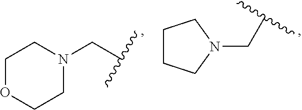

—CH₂—NH(CH₃), —CH₂—NH(—CH(CH₃)₂), —CH₂—N(CH₃)₂, or —CH₂P(O)(CH₃)₂.
Aspect 38. The compound of any one of aspects 1-31, wherein R² is —NR³R⁴.
Aspect 39. The compound of aspect 38, wherein R³ is H.
Aspect 40. The compound of aspect 38, wherein R³ is C₁-C₆alkyl, such as CH₃.
Aspect 41. The compound of any one of aspects 38-40, wherein R⁴ is H.
Aspect 42. The compound of any one of aspects 38-40, wherein R⁴ is optionally substituted C₁-C₆alkyl.
Aspect 43. The compound of aspect 42, wherein R⁴ is unsubstituted C₁-C₆alkyl, such as CH₃ or
   —CH₂CH₃, —CH(CH₃)₂.
Aspect 44. The compound of aspect 42, wherein R⁴ is —CH₂CH(CH₃)₂, —CH₂CH₂CH₃, —CH(CH₃)CH₂CH₃,

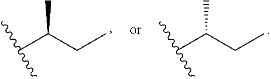

Aspect 45. The compound of aspect 42, wherein R⁴ is substituted C₁-C₆alkyl.
Aspect 46. The compound of aspect 45, wherein R⁴ is —CH₂-cyclopropyl, —CH₂CH₂CHF₂, —CH₂CHF₂, —CH₂CH₂OH, —CH₂CH₂OCH₃, —CH₂CH₂O-iso-Pr, —CH₂C(CH₃)₂OH, —CH₂CHCH₃OH, —CH₂CHOCH₃, —C(O)CH₂N(C₁-C₆alkyl)₂,

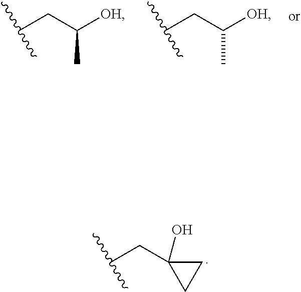

Aspect 47. The compound of any one of aspects 38-40, wherein R⁴ is optionally substituted C₂-C₆alkenyl, such as —CH₂CH═CH₂.
Aspect 48. The compound of any one of aspects 38-40, wherein R⁴ is C₃-C₅cycloalkyl, such as cyclobutyl.
Aspect 49. The compound of any one of aspects 38-40, wherein R⁴ is 3- to 6-membered heterocycloalkyl.
Aspect 50. The compound of aspect 49, wherein the 3- to 6-membered heterocycloalkyl is oxetan-3-yl, thietane-3-yl-1,1-dioxide, tetrahydrofuran-3-yl,

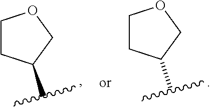 [SMILES: *[C@H]1CCOC1 ; *[C@@H]1CCOC1]

Aspect 51. The compound of any one of aspects 38-40, wherein R⁴ is C(O)CH₂NR^{4d}R^{4e}, such as, C(O)CH₂N(C₁-C₆alkyl)₂ or C(O)CH₂N(CH₃)₂.
Aspect 52. The compound aspect 38, wherein R³ and R⁴, together with the N atom to which they are both attached, form a 3- to 6-membered heterocycloalkyl optionally substituted with one or more substituents that are each C₁-C₆alkyl, C₁-C₆alkoxyl, F, or OH.
Aspect 53. The compound of aspect 38, wherein R³ and R⁴, together with the N atom to which they are both attached, form an unsubstituted 3- to 6-membered heterocycloalkyl, such as pyrrolidine-1-yl, azetidine-1-yl, or morpholin-4-yl.
Aspect 54. The compound of aspect 38, R³ and R⁴, together with the N atom to which they are both attached, form a substituted 3- to 6-membered heterocycloalkyl, such as 3,3-dimethyl-azetidin-1-yl, 3-hydroxy-3-methylazetidin-1-yl, 1-methyl-4-azaphosphinan-4-yl 1-oxide, or 3-methoxy-3-methylazetidin-1-yl.
Aspect 55. The compound of any one of aspects 1-31, wherein R² is —OR⁴a.
Aspect 56. The compound of aspect 55, wherein R^{4a} is H.
Aspect 57. The compound of aspect 55, wherein R⁴ is optionally substituted C₁-C₆alkyl.
Aspect 58. The compound of aspect 57, wherein R⁴ is unsubstituted C₁-C₆alkyl, such as —CH(CH₃).
Aspect 59. The compound of aspect 57, wherein R⁴ is substituted C₁-C₆alkyl.
Aspect 60. The compound of aspect 59, wherein R^{4a} is —CH₂CH₂OH, or —CH₂C(CH₃)₂OH.
Aspect 61. The compound of any one of aspects 1-31, wherein R² is —P(O)R^{4b}R^{4c}.
Aspect 62. The compound of any one of aspects 1-31, wherein R² is —SO₂R³.
Aspect 63. The compound of any one of aspects 1-27, wherein R¹ and R², together with the carbon atom to which they are both attached, form a 3-5 membered cycloalkyl ring, such as a cyclopropyl ring.
Aspect 64. The compound of aspect 13, wherein the compound of formula (IA) is a compound of formula (IA-1):

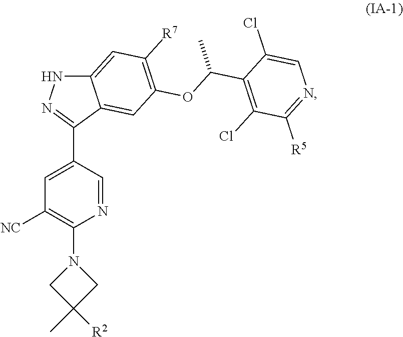

or a pharmaceutically acceptable salt thereof.
Aspect 65. The compound of aspect 13, wherein the compound of formula (IA) is a compound of formula (IA-2):

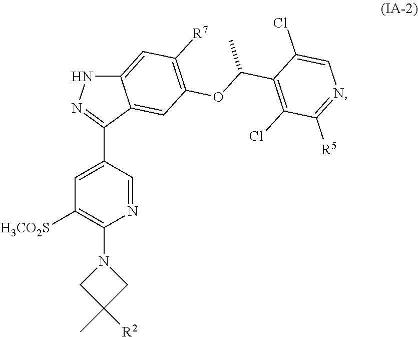

or a pharmaceutically acceptable salt thereof.
Aspect 66. The compound of aspect 13, wherein the compound of formula (IA) is a compound of formula (IA-3):

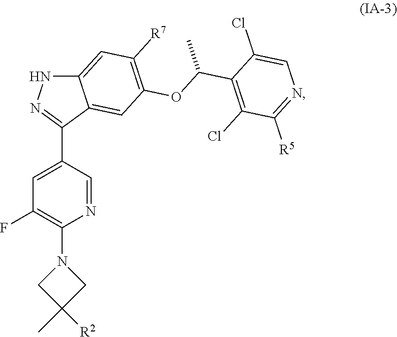

or a pharmaceutically acceptable salt thereof.
Aspect 67. The compound of aspect 13, wherein the compound of formula (IA) is a compound of formula (IA-4):

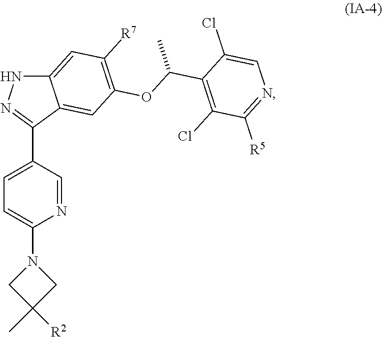

or a pharmaceutically acceptable salt thereof.
Aspect 68. The compound of aspect 13, wherein the compound of formula (IA) is a compound of formula (IA-5):

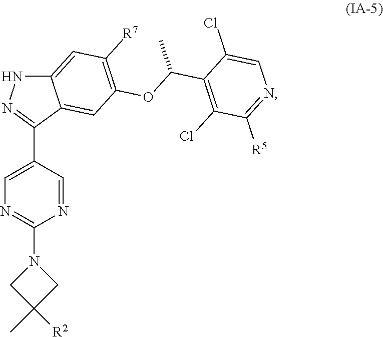

or a pharmaceutically acceptable salt thereof.
Aspect 69. The compound of any one of aspects 64 to 68, wherein R⁵ is H.
Aspect 70. The compound of any one of aspects 64 to 68, wherein R⁵ is CH₃.
Aspect 71. The compound of any one of aspects 64 to 70, wherein R⁷ is H.
Aspect 72. The compound of any one of aspects 64 to 70, wherein R⁷ is F.
Aspect 73. The compound of any one of aspects 64 to 70, wherein R⁷ is OCH₃.
Aspect 74. The compound of any one of aspects 64 to 73, wherein R² is optionally substituted C₁-C₆alkyl.
Aspect 75. The compound of aspect 74, wherein R² is CH₃, —CH₂—NHSO₂(CH₃), —CH₂—N(CH₃)SO₂(CH₃) O,

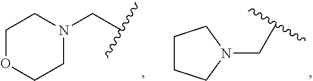 [SMILES: *CN1CCOCC1 ; *CN1CCCC1]

—CH₂—NH(CH₃), —CH₂—NH(—CH(CH₃)₂), —CH₂—N(CH₃)₂, or —CH₂—P(O)(CH₃)₂.
Aspect 76. The compound of any one of aspects 64 to 73, wherein R² is —NR³R⁴.
Aspect 77. The compound of aspect 76, wherein R³ is H.
Aspect 78. The compound of aspect 76, wherein R³ is CH₃.
Aspect 79. The compound of any one of aspects 76 to 78, wherein R⁴ is H.
Aspect 80. The compound of any one of aspects 76 to 78, wherein R⁴ is CH₃, —CH₂CH₃, —CH(CH₃), —CH₂-cyclopropyl, —CH₂CH₂CHF₂, —CH₂CHF₂, —CH₂CH₂OH, —CH₂C(CH₃)₂OH, —CH₂CHOCH₃, —C(O)CH₂N(CH₃)₂,

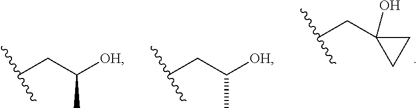

Aspect 81. The compound of any one of aspects 76 to 78, wherein R⁴ is —CH₂CH(CH₃)₂, —CH₂CH₂CH₃, —CH(CH₃)CH₂CH₃,

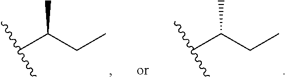

Aspect 82. The compound of any one of aspects 76 to 78, wherein R⁴ is —CH₂CH═CH₂.
Aspect 83. The compound of any one of aspects 76 to 78, wherein R⁴ is cyclopropyl, cyclobutyl, or cyclopentyl.
Aspect 84. The compound of any one of aspects 76 to 78, wherein R⁴ is oxetan-3-yl, thietane-3-yl-1,1-dioxide, tetrahydrofuran-3-yl,

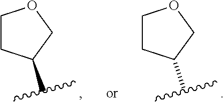 [SMILES: *[C@H]1CCOC1 ; *[C@@H]1CCOC1]

Aspect 85. The compound of any one of aspects 76 to 78, wherein R⁴ is C(O)CH₂N(CH₃)₂.
Aspect 86. The compound of aspect 76, wherein R³ and R⁴, together with the N atom to which they are both attached, form a 3- to 6-membered heterocycloalkyl optionally substituted with one or more substituents that are each C₁-C₆alkyl, C₁-C₆alkoxyl, or OH.
Aspect 87. The compound of aspect 86, wherein R³ and R⁴, together with the N atom to which they are both attached, form a 3,3-dimethyl-azetidin-1-yl, 3-hydroxy-3-methylazetidin-1-yl, 1-methyl-4-azaphosphinan-4-yl 1-oxide, 1-methyl-4-azaphosphinan-4-yl 1-oxide, or 3-methoxy-3-methylazetidin-1-yl.
Aspect 88. The compound of any one of aspects 64 to 73, R² is —P(O)R^{4b}R^{4c}
Aspect 89. The compound of any one of aspects 64 to 73, R² is —OR^{4a}.
Aspect 90. The compound of aspect 89, wherein R^{4a} is —CH(CH₃), —CH₂CH₂OH, —CH₂C(CH₃)₂OH, cyclopropyl, cyclobutyl, or cyclopentyl.
Aspect 91. The compound of aspect 13, wherein the compound of formula (IA) is a compound of formula (IA-6):

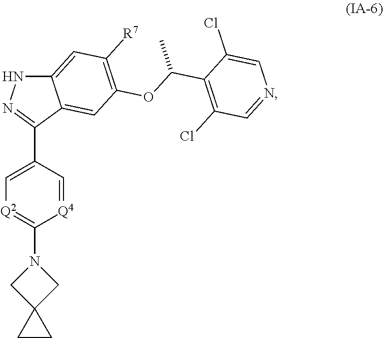

or a pharmaceutically acceptable salt thereof.
Aspect 92. A pharmaceutical composition comprising a compound of any one of aspects 1-91, or a pharmaceutically acceptable salt thereof, and a pharmaceutically acceptable excipient.
Aspect 93. A method of treating a disease or disorder in a subject in need thereof comprising administering to the subject a compound of any one of aspects 1 to 91, or a pharmaceutically acceptable salt thereof.
Aspect 94. The method of aspect 93, wherein the disease or disorder is cancer.
Aspect 95. The method of aspect 94, wherein the cancer is urothelial carcinoma, hepatocellular carcinoma, breast carcinoma, endometrial adenocarcinoma, ovarian carcinoma, primary glioma, cholangiocarcinoma, gastric adenocarcinoma, non-small cell lung carcinoma, pancreatic exocrine carcinoma, oral cancer, prostate cancer, bladder cancer, colorectal carcinoma, renal cell carcinoma, neuroendocrine carcinoma, myeloproliferative neoplasms, head and neck (squamous), melanoma, leiomyosarcoma, and/or sarcomas.
Aspect 96. The method of aspect 95, wherein the cancer is bladder cancer.
Aspect 97. The method of aspect 95, wherein the cancer is urothelial carcinoma.
Aspect 98. The method of aspect 95, wherein the cancer is hepatocellular carcinoma.
Aspect 99. The method of any one of aspects 94 to 98, wherein the cancer is an FGFR-mutant cancer.
Aspect 100. The method of aspect 93, wherein the disease or disorder is a developmental disorder.
Aspect 101. The method of aspect 100, wherein the developmental disorder is achondroplasia.
Aspect 102. A method of inhibiting FGFR in a cell comprising contacting the cell with a compound of any one of aspects 1 to 91.

### EXAMPLES

The examples and preparations provided below further illustrate and exemplify the compounds of the present invention and methods of preparing such compounds. It is to be understood that the scope of the present invention is not limited in any way by the scope of the following examples and preparations.

In several embodiments, where single enantiomers are provided, the enantiomers may be separated by conventional means (chiral chromatography, preparing diastereomeric salts, chiral derivatization, crystallization, enzymatic reactions, etc.). In several embodiments, a chiral intermediate compound is purified to prepare an enantiomerically pure (or substantially enantiomerically pure, enantiomerically enriched, etc.) intermediate.

### Scheme for 6-Methoxyindazole Example 3

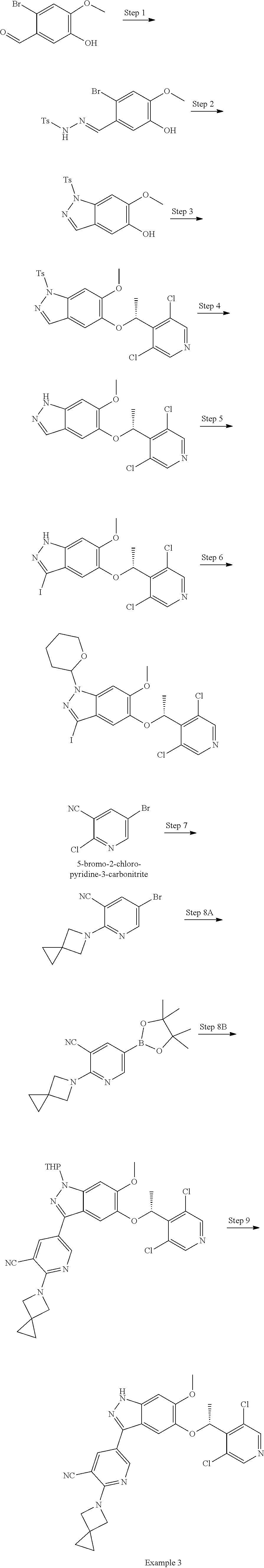

### Example 3. (R)-5-(5-(1-(3,5-dichloropyridin-4-yl)ethoxy)-6-methoxy-1H-indazol-3-yl)-2-(5-azaspiro[2.3]hexan-5-yl)nicotinonitrile

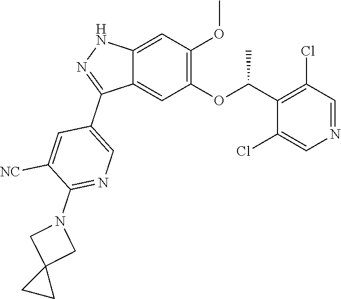 [SMILES: COc1cc2[nH]nc(-c3cnc(N4CC5(CC5)C4)c(C#N)c3)c2cc1O[C@H](C)c1c(Cl)cncc1Cl]

Step 1. (E)-N′-(2-Bromo-5-hydroxy-4-methoxybenzylidene)-4-methylbenzenesulfonohydrazide. p-Toluenesulfonyl hydrazide (0.56 g, 3.0 mmol, 1.0 equiv) was added to a solution of 2-bromo-5-hydroxy-4-methoxy-benzaldehyde (0.7 g, 3.0 mmol, 1.0 equiv) in methanol (7.0 mL) at rt. The resulting mixture was heated at 60° C. for 2 h. The reaction was cooled to rt and the solvent was removed under reduced pressure. The residue was dissolved in ethyl acetate (20 mL), then heptanes (80 mL) was added to give a light-yellow solid (1.21 g, 100%). LCMS m/z=399.0 (M+H).

Step 2. 6-Methoxy-1-tosyl-1H-indazol-5-ol. Copper(I) oxide (0.22 g, 1.5 mmol, 0.5 equiv) was added to a solution of (E)-N′-(2-Bromo-5-hydroxy-4-methoxybenzylidene)-4-methylbenzenesulfonohydrazide (1.2 g, 3.0 mmol, 1.0 equiv) in isoamyl alcohol (30 mL) at room temperature. After heating at 132° C. for 2 hours, the mixture was cooled to room temperature and diluted with water (80 mL). The mixture was extract with ethyl acetate (4×50 mL). The combined organic layers were dried over sodium sulfate and filtered. The filtrate was concentrated onto silica gel (8.0 g) and purified on a Biotage automated purification system (Biotage Sfar Silica, 50 g; 0% to 100% ethyl acetate in heptanes) to give a light yellow solid (0.66 g, 70% yield). LCMS m/z=319.1 (M+H).

Step 3. (R)-5-(1-(3,5-Dichloropyridin-4-yl)ethoxy)-6-methoxy-1-tosyl-1H-indazole. (1S)-1-(3,5-dichloro-4-pyridyl)ethyl] methanesulfonate (0.57 g, 2.1 mmol, 1.0 equiv) and cesium carbonate (1.03 g, 3.2 mmol, 1.5 equiv) were added to a solution of 6-methoxy-1-tosyl-1H-indazol-5-ol (0.67 g, 2.1 mmol, 1.0 equiv) in acetonitrile (21 mL) at room temperature. After heating at 90° C. overnight, the mixture was cooled to room temperature and concentrated onto silica gel (6.0 g) under reduced pressure. The product was purified on a Biotage automated purification system (Sorbtech silica, 40 g), eluting with a gradient of 0% to 60% ethyl acetate in heptanes to give a white solid (0.71 g, 70% yield). LCMS m/z=492.1 (M+H).

Step 4. (R)-5-(1-(3,5-Dichloropyridin-4-yl)ethoxy)-6-methoxy-1H-indazole. 1M Tetrabutylammonium fluoride in THE (7.2 mL, 7.2 mmol, 18.0 equiv) was added to a solution of (R)-5-(1-(3,5-dichloropyridin-4-yl)ethoxy)-6-methoxy-1-tosyl-1H-indazole (0.20 g, 0.4 mmol, 1.0 equiv) in tetrahydrofuran (4 mL) at room temperature. After heating at 50° C. for 4 days, the solvent was removed under reduced pressure. The residue was concentrated onto silica gel (2.0 g) and purified on a Biotage automated purification system (Sorbtech silica, 12 g), eluting with a gradient of 0% to 100% ethyl acetate in heptanes to give a white solid (74.7 mg, 54%). LCMS m/z=338.0 (M+H).

Step 5. (R)-5-(1-(3,5-Dichloropyridin-4-yl)ethoxy)-3-iodo-6-methoxy-1H-indazole. Potassium hydroxide (27.9 mg, 0.50 mmol, 2.25 equiv) and iodine (84.1 mg, 0.33 mmol, 1.5 equiv) were added to a solution of (R)-5-(1-(3,5-dichloropyridin-4-yl)ethoxy)-6-methoxy-1H-indazole (74.7 mg, 0.22 mmol, 1.0 equiv) in N,N-dimethylformamide (2.2 mL) at 0° C. The resulting mixture was allowed to warm up to room temperature and stirred overnight. The reaction was diluted with ethyl acetate (10 mL) and washed with water (4×5 mL). The organic layer was dried over sodium sulfate and concentrated onto silica gel (1.5 g) under reduced pressure. The product was purified on a Biotage automated purification system (Sorbtech silica, 12 g), eluting with a gradient of 0% to 100% ethyl acetate in heptanes to give an off-white solid (80 mg, 77%). LCMS m/z=463.9 (M+H).

Step 6. 5-((R)-1-(3,5-Dichloropyridin-4-yl)ethoxy)-3-iodo-6-methoxy-1-(tetrahydro-2H-pyran-2-yl)-1H-indazole. 5-[(1R)-1-(3,5-Dichloro-4-pyridyl)ethoxy]-3-iodo-6-methoxy-1H-indazole (0.5 g, 1.1 mmol, 1 equiv) was treated with 3,4-dihydro-2H-pyran (0.2 mL, 2.2 mmol, 2 equiv) and p-toluenesulfonic acid monohydrate (10 mg, 0.05 mmol, 0.05 equiv) in anhydrous dichloromethane (6 mL) at room temperature overnight. The mixture was diluted with dichloromethane (8 mL) and washed with water (8 mL). The organic layer was dried over sodium sulfate, filtered and concentrated under reduced pressure onto silica gel (6 g). The residue was purified on a Biotage automated chromatography system (Sorbtech, 12 g silica gel column), eluting with a gradient of 0 to 20% ethyl acetate in heptanes to give a white solid (510 mg, 86% yield). m/z=548 (M+H).

Step 7. 5-Bromo-2-(5-Azaspiro[2.3]hexan-5-yl)nicotinonitrile. 5-Bromo-2-chloropyridine-3-carbonitrile (0.50 g, 2.3 mmol, 1 equiv) in acetonitrile (13 mL) was treated with N,N-diisopropylethylamine (1.2 mL, 6.4 mmol, 3 equiv) and 5-azaspiro[2.3]hexane hydrochloride (0.30 g, 2.5 mmol, 1.1 equiv) at 60° C. overnight. After cooling to room temperature, the mixture was diluted with water (50 mL) and extracted with ethyl acetate (2×50 mL). The combined organic layers were dried over sodium sulfate, filtered and concentrated under reduced pressure, then azeotrope with heptanes (20 mL) and dried under vacuum at 40° C. for 2 hours to give a yellow solid (0.57 g, 94%). LCMS m/z=264 (M+H).

Step 8A. 5-(5-((R)-1-(3,5-Dichloropyridin-4-yl)ethoxy)-6-methoxy-1-(tetrahydro-2H-pyran-2-yl)-1H-indazol-3-yl)-2-(5-azaspiro[2.3]hexan-5-yl)nicotinonitrile. 5-Bromo-2-(5-azaspiro[2.3]-hexan-5-yl)nicotinonitrile (264 mg, 1.0 mmol, 1 equiv) in 1,4-dioxane (3.0 mL) was treated with [1,1′-bis(diphenylphosphino)-ferrocene]dichloropalladium(II) (51 mg, 70.0 μmol, 0.07 equiv), potassium acetate (0.29 g, 3.0 mmol, 3 equiv) and bis(pinacolato)diboron (508 mg, 2.0 mmol, 2 equiv) at 100° C. for 3 hours to give 2-(5-azaspiro[2.3]hexan-5-yl)-5-(4,4,5,5-tetramethyl-1,3,2-dioxaborolan-2-yl)pyridine-3-carbonitrile, as confirmed by LCMS analysis (mz=312 (M+H)). Step 8B. Additional [1,1′-bis(diphenylphosphino)ferrocene]palladium(II) dichloride (51 mg, 70.0 μmol, 0.07 equiv), potassium carbonate (0.28 g, 2.0 mmol, 2.0 equiv), 5-((R)-1-(3,5-dichloropyridin-4-yl)ethoxy)-3-iodo-6-methoxy-1-(tetrahydro-2H-pyran-2-yl)-1H-indazole (0.41 g, 0.75 mmol, 0.75 equiv), and water (0.3 mL) were added and the mixture was heated at 100° C. overnight. After cooling to room temperature, the reaction was diluted with water (20 mL) and extracted with ethyl acetate (4×20 mL). The combined organic layers were dried over sodium sulfate, filtered and concentrated under reduced pressure. The residue was purified on an Interchim automated chromatography system (Sorbtech 25 g silica gel column), eluting with a gradient of 0 to 100% ethyl acetate in heptanes to give a yellow oil (305 mg, 66%). LCMS m/z=605.2 (M+H).

Step 9. (R)-5-(5-(1-(3,5-Dichloropyridin-4-yl)ethoxy)-6-methoxy-1H-indazol-3-yl)-2-(5-azaspiro[2.3]hexan-5-yl)nicotinonitrile trifluoroacetic acid salt. Trifluoroacetic acid (3.2 mL) was added to a solution of product step 8 (320 mg) in dichloromethane (3.2 mL) at room temperature. After stirring at room temperature for 4 hours, the volatiles were removed under reduced pressure and the residue was purified on an Interchim automated chromatography system (RediSep Rf GOLD 15 g HP C18 column), eluting with a gradient of 0 to 100% acetonitrile in water to give the product as a TFA light-yellow solid (110 mg, trifluoroacetic acid salt, 34%). LCMS m/z=521.1 (free base) (M+H); 1H NMR (400 MHz, DMSO-d6) δ=12.96 (s, 1H), 8.73 (d, J=2.3 Hz, 1H), 8.59 (s, 2H), 8.09 (d, J=2.3 Hz, 1H), 7.05 (s, 1H), 7.01 (s, 1H), 6.03-5.96 (m, 1H), 5.75 (s, 1H), 4.38 (s, 4H), 3.86 (s, 3H), 1.76 (d, J=6.6 Hz, 3H), 0.73 (s, 4H).

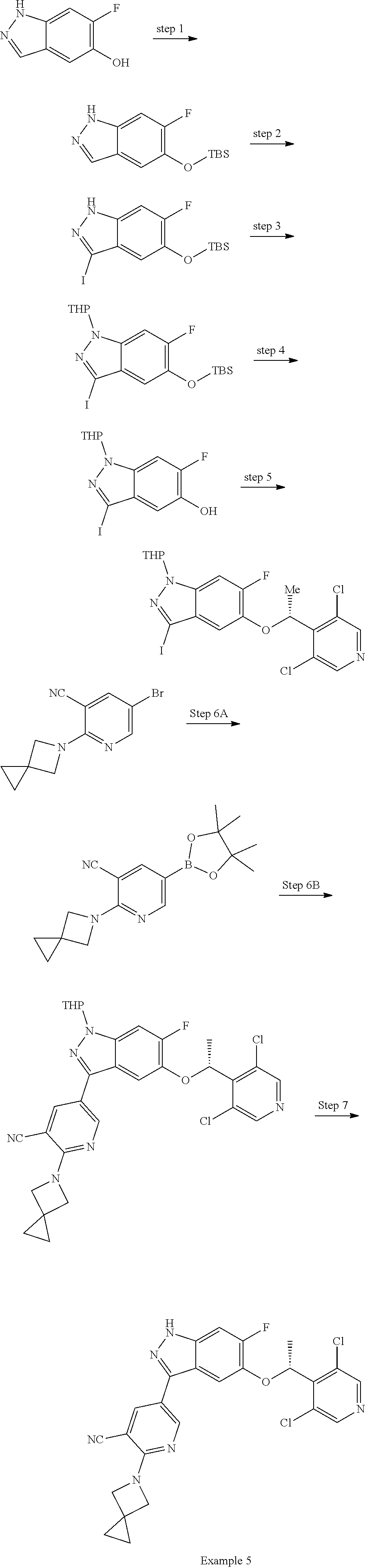

### Example 5

Step 1. 5-((tert-Butyldimethylsilyl)oxy)-6-fluoro-1H-indazole. Imidazole (10 g, 148 mmol) and tert-butyldimethylsilyl chloride (10.7 g, 71 mmol, 1.2 equiv) were sequentially added at 0° C. to a solution of 6-fluoro-1H-indazol-5-ol (9 g, 59 mmol, 1.0 equiv) in N,N-dimethylformamide (59 mL). After stirring at room temperature for 3 hours. The mixture was diluted with ethyl acetate (120 mL), washed with water (4×60 mL) and the organic layer was dried over sodium sulfate, filtered and concentrated under reduced pressure to give a brown oil (15.1 g, 99%). LCMS (ESI) m/z=267.1 (M+H). This material was used directly in the next step.

Step 2. 5-((tert-Butyldimethylsilyl)oxy)-6-fluoro-3-iodo-1H-indazole. 5-((tert-Butyldimethylsilyl)oxy)-6-fluoro-1H-indazole (15.1 g, 57 mmol, 1.0 equiv) in dichloromethane (590 mL) was treated with potassium hydroxide (7.5 g, 128 mmol, 2.25 equiv) and iodine (22.6 g, 85 mmol, 1.5 equiv) at room temperature. After stirring overnight, the reaction was diluted with dichloromethane (1 L) and washed with water (1 L). The organic layer was separated, filtered through a silica gel pad (20 g), which was washed with a 1:1 mixture of ethyl acetate in heptanes (1 L). The filtrate was dried over sodium sulfate, filtered, and concentrated under reduced pressure to give a brown oil (16.5 g, 71% crude yield over 2 steps). LCMS m/z=393.0 (M+H). This material was used directly in the next step.

Step 3. 5-((tert-Butyldimethylsilyl)oxy)-6-fluoro-3-iodo-1-(tetrahydro-2H-pyran-2-yl)-1H-indazole. 5-((tert-Butyldimethylsilyl)oxy)-6-fluoro-3-iodo-1H-indazole (16.5 g, 42 mmol, 1.0 equiv) in dichloromethane (420 mL) was treated with 3,4-dihydro-2H-pyran (7.7 mL, 84 mmol, 2.0 equiv) and p-toluenesulfonic acid (0.4 g, 2 mmol, 0.05 equiv) at room temperature. After stirring overnight, the reaction was diluted with dichloromethane (400 mL) and washed with a saturated sodium bicarbonate (100 mL). The organic layer was dried over sodium sulfate, filtered and concentrated under reduced pressure onto silica gel (60 g). The residue was purified on a Biotage automated chromatography system (Biotage Sfar HC, 200 g, silica gel), eluting with a gradient of 0 to 20% ethyl acetate in heptanes to give a yellow oil (11.2 g, 56% yield over 3 steps). LCMS m/z=477.1 (M+H).

Step 4. 6-Fluoro-3-iodo-1-(tetrahydro-2H-pyran-2-yl)-1H-indazol-5-ol. 5-((tert-Butyldimethylsilyl)oxy)-6-fluoro-3-iodo-1-(tetrahydro-2H-pyran-2-yl)-1H-indazole (11.2 g, 23.5 mmol, 1.0 equiv) in tetrahydrofuran (235 mL) was treated with 1M tetrabutylammonium fluoride in THE (47 mL, 47 mmol, 2.0 equiv) at 0° C. After stirring at 0° C. for 4 hours, the reaction was diluted with dichloromethane (800 mL) and washed with saturated sodium bicarbonate (300 mL). The organic layer was dried over sodium sulfate, filtered and concentrated under reduced pressure onto silica gel (30 g). The residue was purified on a Biotage automated chromatography system (Biotage Sfar HC, 200 g, silica gel), eluting with a gradient of 0 to 10% methanol in dichloromethane to give a white solid (8.07 g, 95%). LCMS m/z=363.0 (M+H).

Step 5. 5-((R)-1-(3,5-Dichloropyridin-4-yl)ethoxy)-6-fluoro-3-iodo-1-(tetrahydro-2H-pyran-2-yl)-1H-indazole. A mixture of 6-fluoro-3-iodo-1-(tetrahydro-2H-pyran-2-yl)-1H-indazol-5-ol (3.0 g, 8.3 mmol, 1.0 equiv), (1S)-1-(3,5-dichloro-4-pyridyl)ethyl] methanesulfonate (2.3 g, 8.3 mmol, 1.0 equiv) and cesium carbonate (4.1 g, 12.4 mmol, 1.5 equiv) in acetonitrile (82 mL) was heated at 90° C. overnight. After cooling to room temperature, the reaction was diluted with ethyl acetate (200 mL) and washed with water (200 mL). The organic layer was dried over sodium sulfate, filtered and concentrated under reduced pressure onto silica gel (30 g). The residue was purified on a Biotage automated chromatography system (Biotage Sfar HC, 200 g, silica gel), eluting with a gradient of 0 to 100% ethyl acetate in heptanes to give a white solid (3.0 g, 68%). LCMS m/z=M+H.

Step 6. 5-(5-((R)-1-(3,5-Dichloropyridin-4-yl)ethoxy)-6-fluoro-1-(tetrahydro-2H-pyran-2-yl)-1H-indazol-3-yl)-2-(5-azaspiro[2.3]hexan-5-yl)nicotinonitrile. A mixture of 5-bromo-2-(5-Azaspiro[2.3]hexan-5-yl)nicotinonitrile (0.12 g, 0.45 mmol, 1.2 equiv), bis(pinacolato)diboron (0.14 g, 0.54 mmol, 1.5 equiv), potassium acetate (66 mg, 0.67 mmol, 1.8 equiv) and [1,1′-bis(diphenylphosphino)ferrocene]palladium(II) dichloride (32 mg, 0.04 mmol, 0.1 equiv) in 1,4-dioxane (10 mL) was sparged with nitrogen for 10 minutes then heated for 3 hours at 90° C. to give 2-(5-azaspiro[2.3]hexan-5-yl)-5-(4,4,5,5-tetramethyl-1,3,2-dioxaborolan-2-yl)pyridine-3-carbonitrile. The mixture was cooled to room temperature, then potassium carbonate (0.16 g, 1.1 mmol, 3 equiv), water (1 mL), [1,1′-bis(diphenylphosphino) ferrocene]palladium(II) dichloride (32 mg, 0.04 mmol, 0.1 equiv) and 5-[(1R)-1-(3,5-dichloro-4-pyridyl)ethoxy]-6-fluoro-3-iodo-1-tetrahydropyran-2-yl-indazole (0.20 g, 1 equiv) were added. The mixture was sparged with nitrogen for 10 minutes then heated overnight at 90° C. The mixture was cooled to room temperature, diluted with water (10 mL) and extracted with ethyl acetate (3×20 mL). The combined organic layers were concentrated under reduced pressure onto silica gel (6 g). The residue was purified on an Biotage automated chromatography system (Sorbtech, 25 g silica gel column), eluting with a gradient of 0 to 50% ethyl acetate in heptanes to give a tan solid (173 mg, 78%) after drying overnight under vacuum at 40° C. LCMS m/z=593 (M+H).

Step 7. (R)-5-(5-(1-(3,5-Dichloropyridin-4-yl)ethoxy)-6-fluoro-1H-indazol-3-yl)-2-(5-azaspiro[2.3]hexan-5-yl)nicotinonitrile. 5-(5-((R)-1-(3,5-Dichloropyridin-4-yl)ethoxy)-6-fluoro-1-(tetrahydro-2H-pyran-2-yl)-1H-indazol-3-yl)-2-(5-azaspiro[2.3]hexan-5-yl)nicotinonitrile (171 mg, 0.28 mmol) was treated with a 1 to 1 mixture of dichloromethane and trifluoroacetic acid (4 mL) overnight. The mixture was concentrated under reduced pressure. The residue was diluted with dichloromethane (20 mL) and saturated sodium bicarbonate (20 mL). The layers were separated and the organic layer was concentrated onto silica gel (4 g). The residue was purified on an Biotage automated chromatography system (Sorbtech, 12 g silica gel column), eluting with a gradient of 0 to 5% methanol in dichloromethane to give a tan solid. The product was further purified by preparative HPLC on a Gilson automated chromatography system (Waters Atlantis T3 Prep OBD column, 5 μm, 19×250 mm), eluting with a gradient of 10 to 100% acetonitrile in water with 0.1% trifluoroacetic acid to give an off-white solid as the TFA salt (25 mg, 14%) after lyophilization. LCMS m/z=509 (M+H); (free base); 1H NMR (400 MHz, DMSO-d6) δ=13.23 (s, 1H), 8.75 (d, J=2.3 Hz, 1H), 8.60 (s, 2H), 8.15 (d, J=2.3 Hz, 1H), 7.47 (d, J=10.9 Hz, 1H), 7.26 (d, J=7.8 Hz, 1H), 6.18 (q, J=6.6 Hz, 1H), 4.40 (s, 4H), 1.80 (d, J=6.7 Hz, 3H), 0.74 (s, 4H).

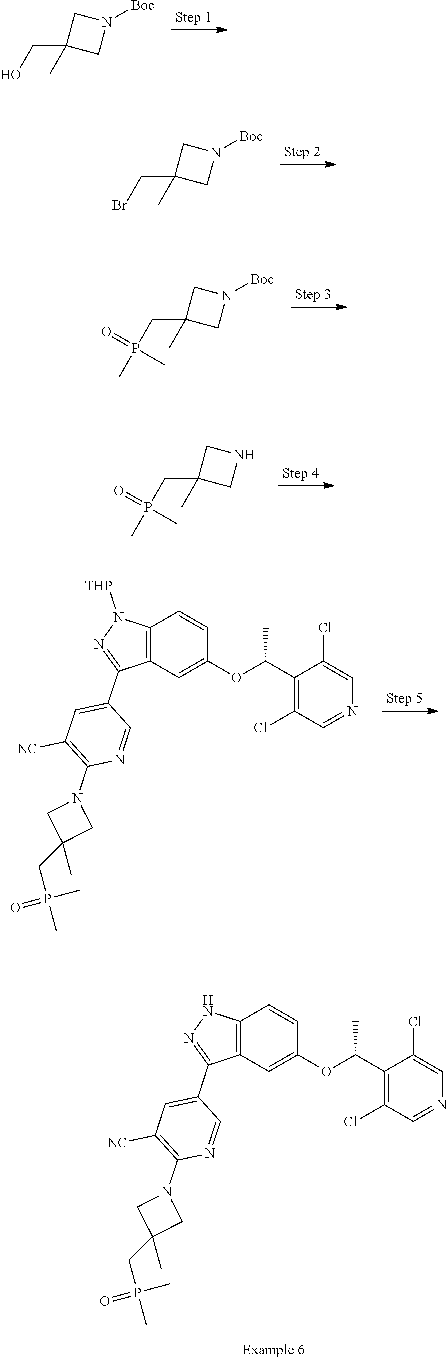

### Example 6. (R)-5-(5-(1-(3,5-dichloropyridin-4-yl)ethoxy)-1H-indazol-3-yl)-2-(3-((dimethylphosphoryl)methyl)-3-methylazetidin-1-yl)nicotinonitrile

Step 1. tert-Butyl 3-(bromomethyl)-3-methyl-azetidine-1-carboxylate. To a solution of tert-butyl 3-(hydroxymethyl)-3-methyl-azetidine-1-carboxylate (500 mg, 2.50 mmol, 1.0 eq) and PPh₃ (1.3 g, 4.97 mmol, 2.0 eq) in DCM (10.0 mL) was added CBr₄ (1.6 g, 4.97 mmol, 2.0 eq) in portions at 0° C. The reaction mixture was stirred at room temperature for 5 h. After the reaction was completed, the mixture was concentrated in vacuum. The crude product was purified by silica gel flash column chromatography (Petroleum Ether/EtOAc=5/1) to give a brown solid (620 mg, 94% yield). LCMS m/z=264.3 (M+H).

Step 2. tert-butyl 3-(Dimethylphosphorylmethyl)-3-methyl-azetidine-1-carboxylate. To a solution of methylphosphonoylmethane (507 mg, 6.50 mmol, 3.0 eq) in THE (2 mL) was added NaHMDS (3.25 mL, 6.50 mmol, 2 M in THF, 3.0 eq) dropwise at −70° C. under N₂ protection. The reaction mixture was stirred at room temperature for 1 h. A solution of tert-butyl 3-(bromomethyl)-3-methyl-azetidine-1-carboxylate (570 mg, 2.17 mmol, 1.0 eq) in THE (3 mL) was added dropwise at 0° C. under N₂ protection and stirred for 4 h. After the reaction was completed, the reaction was quenched with saturated NH₄Cl aqueous solution (20 mL) at 0° C. and extracted with EtOAc (30 mL×3). The combined organic layers were washed with brine (30 mL×2), dried over Na₂SO₄ and concentrated to give an off-white semi-solid (500 mg, 89% yield). LCMS m/z=262.3 (M+H).

Step 3. 3-(Dimethylphosphorylmethyl)-3-methyl-azetidine. To a solution of tert-butyl 3-(dimethylphosphorylmethyl)-3-methyl-azetidine-1-carboxylate (450 mg, 1.73 mmol, 1.0 eq) in DCM (10.0 mL) was added TFA (2.0 mL) and stirred at room temperature for 3 h. The mixture was concentrated to give a yellow oil as the TFA salt. LCMS m/z=162.3 (M+H).

Step 4. 5-[5-[(1R)-1-(3,5-dichloro-4-pyridyl)ethoxy]-1-tetrahydropyran-2-yl-indazol-3-yl]-2-[3-(dimethylphosphorylmethyl)-3-methyl-azetidin-1-yl]pyridine-3-carbonitrile. To a solution of 5-[5-[(1R)-1-(3,5-dichloro-4-pyridyl)ethoxy]-1-tetrahydropyran-2-yl-indazol-3-yl]-2-fluoro-pyridine-3-carbonitrile (190 mg, 0.37 mmol, 1.0 eq) and 3-(dimethylphosphorylmethyl)-3-methyl-azetidine TFA salt (150 mg) in DMSO (5 mL) was added DIEA (240 mg, 1.86 mmol, 5.0 eq). The reaction mixture was stirred for 16 h at 90° C. After the reaction was completed, the reaction was diluted with EtOAc (20 mL), washed with brine (20 mL×3) and concentrated. The crude product was purified by prep-TLC (DCM/MeOH=15/1) to give a yellow solid (120 mg, 50% yield). LCMS m/z=653.2 (M+H).

Step 5. (R)-5-(5-(1-(3,5-Dichloropyridin-4-yl)ethoxy)-6-methoxy-1H-indazol-3-yl)-2-(3-((dimethylphosphoryl)methyl)-3-methylazetidin-1-yl)nicotinonitrile. To a solution of product step 4 (110 mg, 0.17 mmol, 1.0 eq) in DCM (2 mL) was added TFA (0.4 mL) and stirred at room temperature for 3 h. After the reaction was completed, the reaction mixture was concentrated in vacuum. The residue was adjusted pH to 7-8 with saturated NaHCO₃ solution, and extracted with DCM (10 mL×3). The combined organic layers were concentrated in vacuum. The crude product was purified by Prep-HPLC (Prep-C18, 5 μM Triart column, 20×150 mm, YMC-Actus; gradient elution of 15% MeCN in water to 30% MeCN in water with 0.05% NH₃·H₂O over a 11 min period to give a white solid (58 mg, 61% yield). LCMS m/z=569.4 (M+H); 1H NMR (400 MHz, DMSO-d6) δ13.17 (brs, 1H), 8.76 (d, J=2.4 Hz, 1H), 8.57 (s, 2H), 8.13 (d, J=2.4 Hz, 1H), 7.48 (d, J=8.8 Hz, 1H), 7.15 (d, J=2.0 Hz, 1H), 7.10 (dd, J=8.8 Hz, 2.4 Hz, 1H), 6.13 (q, J=6.8 Hz, 1H), 4.27 (d, J=8.4 Hz, 2H), 4.03 (d, J=8.8 Hz, 2H), 2.23 (d, J=10.8 Hz, 2H), 1.75 (d, J=6.8 Hz, 3H), 1.56 (s, 3H), 1.46 (s, 3H), 1.43 (s, 3H).

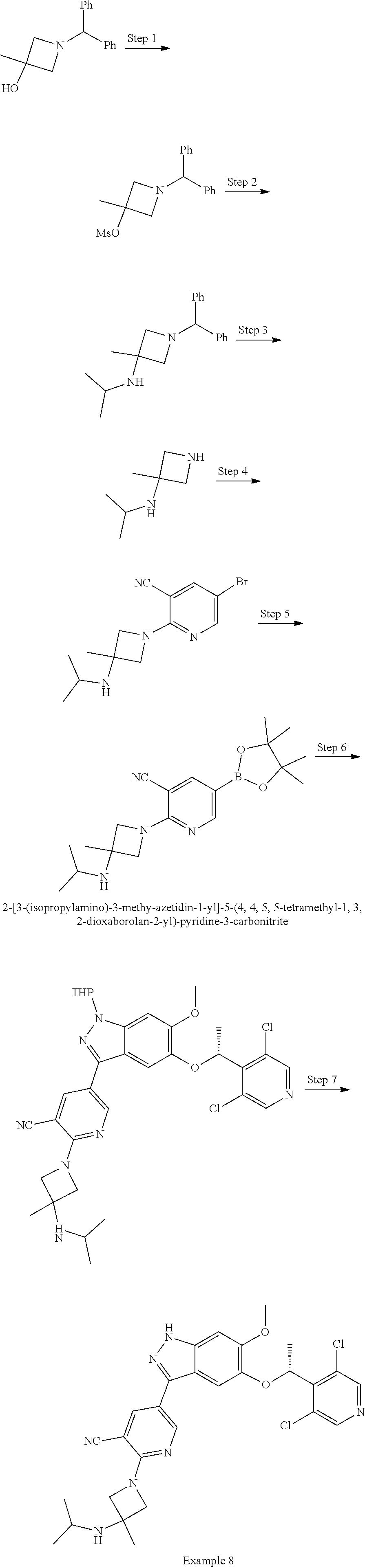

### Example 8

Step 1. 1-Benzhydryl-3-methylazetidin-3-yl methanesulfonate. Methanesulfonyl chloride (6.2 mL, 80 mmol, 2 equiv) was added dropwise to a solution of 1-benzhydryl-3-methyl-azetidin-3-ol (10.13 g, 40 mmol, 1 equiv) and triethylamine (14.0 mL, 100 mmol, 2.5 equiv) in anhydrous dichloromethane (150 mL) at 0° C. After stirring at 0 to 5° C. for 6 hours, water (50 mL) was added and the layers were separated. The aqueous layer was extracted with dichloromethane (3×50 mL). The combined organic layers were washed with saturated brine (80 mL), dried over sodium sulfate, filtered and concentrated under reduced pressure. The residue was purified on an Interchim automated chromatography system (220 g Sorbtech silica gel column), eluting with a gradient of 0 to 40% ethyl acetate in heptanes to give a white solid (4.63 g, 35% yield). LCMS m/z=332 (M+H).

Step 2. 1-Benzhydryl-N-isopropyl-3-methylazetidin-3-amine dihydrochloride. A mixture of 1-benzhydryl-3-methylazetidin-3-yl methanesulfonate (4.14 g, 12.491 mmol, 1 equiv) and isopropylamine (6.44 mL, 75 mmol, 6 equiv) in 2-MeTHF (60 mL) in a sealed pressure bottle was heated at 80° C. for 24 hours. LCMS analysis indicated that the reaction was complete. The mixture was concentrated under reduced pressure, and the residue was suspended in ethyl acetate (100 mL). The solid was filtered off and the filtrate was concentrated under reduced pressure. The residue was dissolved in diethyl ether (100 mL) and 2 M HCL in diethyl ether (40 mL, 80 mmol) was added. After stirring at room temperature for 30 minutes, the resulting solid was filtered and dried under vacuum at 40° C. overnight to give a white solid as the di-HCl salt (4.36 g, 95% yield). LCMS m/z=295 (free base) (M+H).

Step 3. N-Isopropyl-3-methylazetidin-3-amine dihydrochloride. A mixture of 1-benzhydryl-N-isopropyl-3-methylazetidin-3-amine dihydrochloride (4.36 g, 11.863 mmol, 1 equiv) and 20% palladium hydroxide (0.87 g, 50% wet) in methanol (440 mL) was hydrogenated at 50 psi at room temperature for 16 hours. The mixture was filtered through Celite, which was washed with methanol (3×40 mL). The filtrate was concentrated under reduced pressure and the residue was triturated with heptane (5×20 mL) to remove by-product diphenylmethane. The residue was dried under vacuum at 40° C. to give a yellow solid containing a small amount of diphenylmethane by-product (2.58 g, >100% yield). LCMS m/z=129 (free base) (M+H).

Step 4. 5-Bromo-2-(3-(isopropylamino)-3-methylazetidin-1-yl)nicotinonitrile. N,N-Diisopropylethylamine (1.74 mL, 10 mmol, 5 equiv) was added to a mixture of N-isopropyl-3-methylazetidin-3-amine dihydrochloride (0.402 g, 2 mmol, 1 equiv) in acetonitrile (10 mL). After stirring at room temperature for 10 minutes, 5-bromo-2-chloro-3-cyano-pyridine (0.435 g, 2 mmol, 1 equiv) was added and the mixture was stirred at room temperature over the weekend. The mixture was pre-absorbed onto Celite (10 g) under reduced pressure and purified on an Interchim automated chromatography system (RediSep Rf GOLD 100 g HP C18 column), eluting with a gradient of 0 to 100% acetonitrile in water, to give a yellow solid (0.36 g, 58% yield). LCMS m/z=310 (M+H).

Step 5. 2-(3-(Isopropylamino)-3-methylazetidin-1-yl)-5-(4,4,5,5-tetramethyl-1,3,2-dioxaborolan-2-yl)nicotinonitrile. A mixture of 5-bromo-2-(3-(isopropylamino)-3-methylazetidin-1-yl)nicotinonitrile (0.36 g, 1.164 mmol, 1 equiv), bis(pinacolato)diboron (0.443 g, 1.746 mmol, 1.5 equiv), potassium acetate (0.228 g, 2.328 mmol, 2 equiv) and [1,1′-bis(diphenylphosphino)-ferrocene]dichloropalladium(II) (85 mg, 0.116 mmol, 0.1 equiv) in 1,4-dioxane (15 mL) was sparged with nitrogen for 15 minutes then heated at 90° C. for 16 hours. LCMS analysis indicated that the reaction was complete with a mixture of borolane and boronic acid. The reaction mixture was cooled to room temperature and used subsequently in the next step. LCMS m/z=275 (boronic acid) and 357 (M+H).

Step 6. 5-(5-((R)-1-(3,5-Dichloropyridin-4-yl)ethoxy)-6-methoxy-1-(tetrahydro-2H-pyran-2-yl)-1H-indazol-3-yl)-2-(3-(isopropylamino)-3-methylazetidin-1-yl)nicotinonitrile. The reaction mixture from previous step (0.588 mmol, 1.5 equiv) in 1,4-dioxane was treated with 5-((R)-1-(3,5-dichloropyridin-4-yl)ethoxy)-3-iodo-6-methoxy-1-(tetrahydro-2H-pyran-2-yl)-1H-indazole (0.215 g, 0.392 mmol, 1 equiv), [1,1′-bis(diphenylphosphino)ferrocene]dichloropalladium(II) (29 mg, 0.05 mmol, 0.1 equiv) and potassium carbonate (0.108 g, 0.784 mmol, 2 equiv) and water (1.0 mL). After sparging with nitrogen for 10 minutes the reaction was heated at 90° C. for 20 hours. The mixture was cooled to room temperature and diluted with ethyl acetate (50 mL) and water (20 mL). The layers were separated and the aqueous layer was extracted with ethyl acetate (3×20 mL). The combined organic layers were washed with saturated brine (20 mL), dried over sodium sulfate, filtered and concentrated under reduced pressure. The crude product was purified on an Interchim automated chromatography system (Sorbtech 40 g silica gel column), eluting with a gradient of 0 to 10% methanol in dichloromethane to give a brown solid (0.250 g, 85% purity, 98% yield for 2 steps). LCMS m/z=650 (M+H).

Step 7. (R)-5-(5-(1-(3,5-Dichloropyridin-4-yl)ethoxy)-6-methoxy-1H-indazol-3-yl)-2-(3-(isopropylamino)-3-methylazetidin-1-yl)nicotinonitrile. Trifluoroacetic acid (2.5 mL) was added to a solution of 5-(5-((R)-1-(3,5-dichloropyridin-4-yl)ethoxy)-6-methoxy-1-(tetrahydro-2H-pyran-2-yl)-1H-indazol-3-yl)-2-(3-(isopropylamino)-3-methylazetidin-1-yl)nicotinonitrile (0.250 g, 0.384 mmol, 1.0 equiv) in dichloromethane (2.5 mL) at room temperature. After stirring at room temperature for 20 hours, the volatiles were removed under reduced pressure. The residue was dry-loaded on celite (8 g) and purified on an InterChim automated chromatography system (RediSep Rf GOLD 50 g HP C18 column), eluting with a gradient of 0 to 100% acetonitrile in water to give a white solid as the TFA salt (96 mg, 44% yield). LCMS m/z=566 (free base) (M+H); 1H NMR (400 MHz, DMSO-d6) δ=9.20 (br s, 2H), 8.79 (d, J=2.2 Hz, 1H), 8.58 (s, 2H), 8.19 (d, J=2.3 Hz, 1H), 7.06 (s, 1H), 7.02 (s, 1H), 6.03-5.97 (m, 1H), 4.49 (d, J=9.7 Hz, 2H), 4.34 (d, J=9.4 Hz, 2H), 3.86 (s, 3H), 3.55 (br s, 1H), 3.33 (br s, 3H), 1.79-1.75 (m, 3H), 1.26 (d, J=6.4 Hz, 6H).

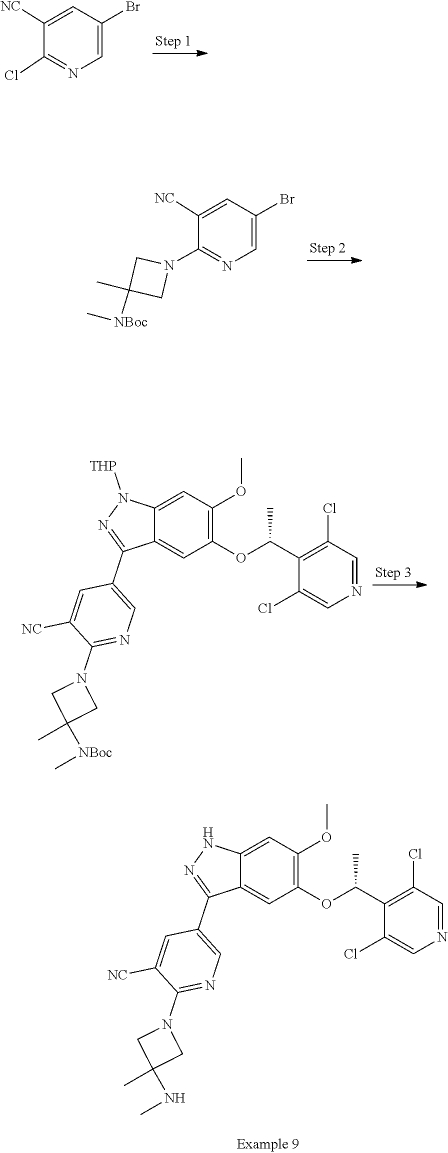

### Example 9. 5-[5-[(1R)-1-(3,5-dichloro-4-pyridyl)ethoxy]-6-methoxy-1H-indazol-3-yl]-2-[3-methyl-3-(methylamino)azetidin-1-yl]pyridine-3-carbonitrile

Step 1. tert-Butyl (1-(5-bromo-3-cyanopyridin-2-yl)-3-methylazetidin-3-yl)(methyl)carbamate. A mixture of 5-bromo-2-chloro-pyridine-3-carbonitrile (326 mg, 1.5 mmol, 1.0 equiv), tert-butyl N-methyl-N-(3-methylazetidin-3-yl)carbamate HCl (355 mg, 1.5 mmol, 1.0 equiv) and N,N-diisopropylethylamine (1.4 mL, 8.1 mmol, 5.3 equiv) in acetonitrile (10 mL) was heated at 70° C. After 24 hours, the reaction was cooled to room temperature and diluted with water (70 mL) and ethyl acetate (50 mL). The layers were separated and the aqueous layer was extracted with ethyl acetate (2×50 mL). The combined organic layers were washed with saturated brine (30 mL), dried over magnesium sulfate, filtered and concentrated to give a yellow oil (460 mg, 80% yield) after drying at 50° C. under vacuum for 16 hours. LCMS m/z=381 (M+H).

Step 2. tert-Butyl (1-(3-cyano-5-(5-((R)-1-(3,5-dichloropyridin-4-yl)ethoxy)-6-methoxy-1-(tetrahydro-2H-pyran-2-yl)-1H-indazol-3-yl)pyridin-2-yl)-3-methylazetidin-3-yl)(methyl)carbamate. tert-Butyl (1-(5-bromo-3-cyanopyridin-2-yl)-3-methylazetidin-3-yl)(methyl)carbamate (150 mg, 0.39 mmol, 1.0 equiv) in 1,4-dioxane (5 mL) was treated with [1,1′-bis(diphenylphosphino)ferrocene]-dichloropalladium(II) (20 mg, 0.03 mmol, 0.07 equiv), bis(pinacolato)diboron (109 mg, 0.43 mmol, 1.1 equiv) and potassium acetate (46 mg, 0.47 mmol, 1.2 equiv). The mixture was sparged with argon for 5 minutes and then heated at 90° C. After 15 hours, the reaction mixture was cooled to room temperature. 5-[(1R)-1-(3,5-Dichloro-4-pyridyl)ethoxy]-3-iodo-6-methoxy-1-tetrahydropyran-2-yl-indazole (215 mg, 0.39 mmol, 1.00 equiv), [1,1′-bis(diphenylphosphino)ferrocene]-dichloropalladium(II) (28 mg, 0.04 mmol, 0.1 equiv), potassium carbonate (162 mg, 1.17 mmol, 3.0 equiv) and water (1.5 mL) were added and the mixture was sparged with argon for 5 minutes. After heating at 90° C. for 16 hours, the reaction mixture was cooled to room temperature and diluted with ethyl acetate (20 mL) and aqueous saturated sodium carbonate solution (35 mL). The layers were separated and the aqueous layer was extracted with ethyl acetate (2×20 mL). The combined organic layers were washed with saturated brine (20 mL), dried over magnesium sulfate, filtered, and concentrated under reduced pressure. The residue was purified on a Biotage automated chromatography system (50 g Biotage, 20 μm silica gel column), eluting with a gradient of 0 to 60% ethyl acetate in heptanes to give a light-yellow oil (110 mg, 38% yield). LCMS m/z=722 (M+H).

Step 3. (R)-5-(5-(1-(3,5-Dichloropyridin-4-yl)ethoxy)-6-methoxy-1H-indazol-3-yl)-2-(3-methyl-3-(methylamino)azetidin-1-yl)nicotinonitrile. A solution of product step 2 (110 mg, 0.15 mmol, 1.0 equiv) in dichloromethane (5 mL) was treated with trifluoroacetic acid (3 mL) at room temperature. After 16 hours, the reaction mixture was cooled to 0° C. and quenched with saturated sodium bicarbonate (50 mL). The mixture was extracted with dichloromethane (2×15 mL). The combined organic layers were washed with saturated brine (20 mL), dried over magnesium sulfate, filtered and concentrated under reduced pressure. The residue was purified on a Biotage automated chromatography system (RediSep Rf GOLD 100 g HP C18 column), eluting with a gradient of 0 to 100% acetonitrile in water. The pure fractions were lyophilized to give a white solid (25 mg, 31% yield). LCMS m/z=538.1 (M+H); 1H NMR (400 MHz, DMSO-d6) δ=12.96 (s, 1H), 8.72 (d, J=2.2 Hz, 1H), 8.59 (s, 2H), 8.07 (d, J=2.4 Hz, 1H), 7.04 (s, 1H), 7.01 (s, 1H), 6.00 (q, J=6.6 Hz, 1H), 4.18-4.08 (m, 2H), 4.08-3.98 (m, 2H), 3.86 (s, 3H), 2.28 (s, 3H), 1.76 (d, J=6.8 Hz, 3H), 1.51-1.38 (m, 3H).

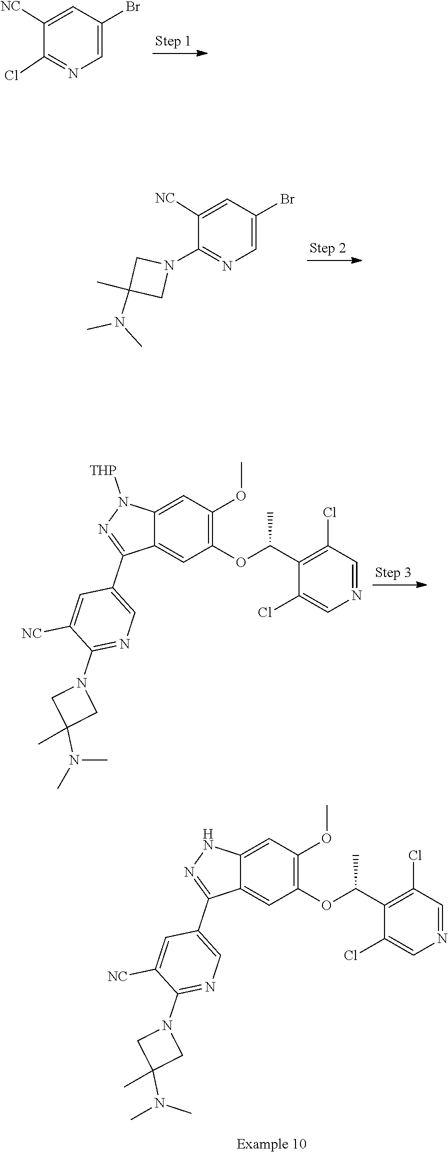

### Example 10. 5-[5-[(1R)-1-(3,5-dichloro-4-pyridyl)ethoxy]-6-methoxy-1H-indazol-3-yl]-2-[3-(dimethylamino)-3-methyl-azetidin-1-yl]pyridine-3-carbonitrile

Step 1. 5-Bromo-2-(3-(dimethylamino)-3-methylazetidin-1-yl)nicotinonitrile. 5-Bromo-2-chloro-pyridine-3-carbonitrile (0.53 g, 2.43 mmol, 1 equiv) in acetonitrile (15 mL) was treated with N,N-3-trimethylazetidin-3-amine HCl (0.5 g, 2.67 mmol, 1.1 equiv) and N,N-diisopropylethylamine (0.94 g, 7.28 mmol, 3 equiv) at 60° C. After 13 hours, the reaction mixture was cooled to room temperature and diluted with water (70 mL) and ethyl acetate (50 mL). The layers were separated and the aqueous layer was extracted with ethyl acetate (2×50 mL). The combined organic layers were washed with saturated brine (30 mL), dried over sodium sulfate, filtered and concentrated under reduced pressure to give a yellow solid (0.65 g, 91% yield). LCMS m/z=295.0 (M+H).

Step 2. 5-(5-((R)-1-(3,5-Dichloropyridin-4-yl)ethoxy)-6-methoxy-1-(tetrahydro-2H-pyran-2-yl)-1H-indazol-3-yl)-2-(3-(dimethylamino)-3-methylazetidin-1-yl)nicotinonitrile. 5-Bromo-2-(3-(dimethylamino)-3-methylazetidin-1-yl)nicotinonitrile (295 mg, 1.0 mmol, 1 equiv) in 1,4-dioxane (3.0 mL) was treated with [1,1′-bis(diphenylphosphino) ferrocene]dichloropalladium(II) (51 mg, 70.0 μmol, 0.07 equiv), potassium acetate (0.29 g, 3.0 mmol, 3 eq), bis(pinacolato)diboron (508 mg, 2.0 mmol, 2 equiv) at 100° C. for 3 hours. LCMS analysis conformed product. Additional 1,1′-Bis(diphenylphosphino)ferrocene]palladium(II) dichloride (51 mg, 70.0 μmol, 0.07 equiv), potassium carbonate (0.28 g, 2.0 mmol, 2.0 equiv), 5-[(1R)-1-(3,5-dichloro-4-pyridyl)ethoxy]-3-iodo-6-methoxy-1-tetrahydropyran-2-yl-indazole (0.41 g, 0.75 mmol, 0.75 equiv) and water (0.3 mL) were then added. After heating at 100° C. overnight, the reaction was cooled to room temperature and diluted with water (20 mL). The mixture was extracted with ethyl acetate (4×20 mL). The combined organic layers were dried over sodium sulfate, filtered and concentrated under reduced pressure. The residue was purified on an Interchim automated chromatography system (Sorbtech 25 g silica-gel column) eluting with a gradient of 0 to 100% ethyl acetate in heptanes to give a dark red oil (340 mg, 57% yield). LCMS m/z=636.3 (M+H).

Step 3. (R)-5-(5-(1-(3,5-Dichloropyridin-4-yl)ethoxy)-6-methoxy-1H-indazol-3-yl)-2-(3-(dimethylamino)-3-methylazetidin-1-yl)nicotinonitrile trifluoroacetic acid salt. Trifluoroacetic acid (3.0 mL) was added to a solution of product step 2 (300 mg) in dichloromethane (3.0 mL) at room temperature. After stirring at room temperature for 3 hours, the volatiles were removed under reduced pressure. The residue was purified on an Interchim automated chromatography system (RediSep Rf GOLD 15 g HP C18 column), eluting with a gradient of 0 to 100% acetonitrile in water to give a light-yellow solid (95 mg, trifluoroacetic acid salt, 33% yield). LCMS m/z=552.1 (M+H); 1H NMR (400 MHz, DMSO-d6) δ=13.04 (br s, 1H), 10.63 (br s, 1H), 8.78 (d, J=2.2 Hz, 1H), 8.59 (s, 2H), 8.20 (d, J=2.3 Hz, 1H), 7.06 (s, 1H), 7.03 (s, 1H), 6.03-5.97 (m, 1H), 4.51 (br d, J=10.1 Hz, 2H), 4.26 (d, J=10.0 Hz, 2H), 3.87 (s, 3H), 2.80 (br s, 6H), 1.77 (d, J=6.6 Hz, 3H), 1.68 (s, 3H).

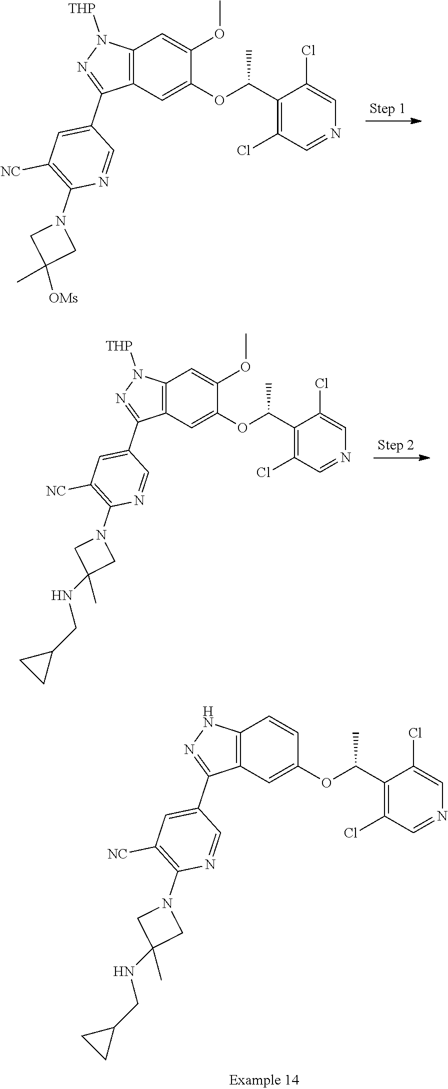

### Example 14. 2-[3-(cyclopropylmethylamino)-3-methyl-azetidin-1-yl]-5-[5-[(1R)-1-(3,5-dichloro-4-pyridyl)ethoxy]-6-methoxy-1H-indazol-3-yl]pyridine-3-carbonitrile

Step 1. To a solution of [1-[3-cyano-5-[5-[(1R)-1-(3,5-dichloro-4-pyridyl)ethoxy]-6-methoxy-1-tetrahydropyran-2-yl-indazol-3-yl]-2-pyridyl]-3-methyl-azetidin-3-yl]methanesulfonate (300 mg, 0.43 mmol, 1.0 eq) and cyclopropylmethylamine (310 mg, 4.36 mmol, 10.0 eq) in ACN (10 mL) was added t-BuONa (126 mg, 1.31 mmol, 3.0 eq). The reaction mixture was stirred at 90° C. for 2 h. After the reaction was complete, EtOAc (20 mL) was added to the mixture, the organic layer was washed with brine (20 mL×2), dried over Na₂SO₄ and concentrated in vacuum. The crude product was purified by Prep-TLC (DCM/MeOH=20/1) to give a yellow oil (190 mg, 66% yield). LCMS m/z=662.3 (M+H).

Step 2. 2-[3-(cyclopropylmethylamino)-3-methyl-azetidin-1-yl]-5-[5-[(1R)-1-(3,5-dichloro-4-pyridyl)ethoxy]-6-methoxy-1H-indazol-3-yl]pyridine-3-carbonitrile To a solution of product step 1 (190 mg, 0.29 mmol, 1.0 eq) in DCM (5 mL) was added TFA (1 mL) and stirred at rt for 16 h. After the completion the reaction mixture was concentrated in vacuum. The crude product was treated with DCM/MeOH (5:1, 10 mL), NaHCO₃ solid was added to the solution and stirred for 20 minutes. The solid was filtered off and filtrate was concentrated. The residue was purified by Prep-HPLC (Prep-C18, 5 μM Triart column, 20×150 mm, YMC-Actus; gradient elution of 40% MeCN in water to 65% MeCN in water over an 8 min period, where both solvents contain 0.05% NH₃·H₂O) to give a white solid (54 mg, 32% yield). LCMS m/z=578.30 (M+H); 1H NMR (400 MHz, DMSO-d6) δ 12.96 (brs, 1H), 8.71 (d, J=2.0 Hz, 1H), 8.59 (s, 2H), 8.06 (d, J=2 Hz, 1H), 7.03 (s, 1H), 7.00 (s, 1H), 6.00 (q, J=6.4 Hz, 1H), 4.14 (d, J=8.4 Hz, 2H), 4.02 (d, J=8.4 Hz, 2H), 3.86 (s, 3H), 2.42 (d, J=6.4 Hz, 2H), 1.76 (d, J=6.4 Hz, 3H), 1.43 (s, 3H), 0.93-0.82 (m, 1H), 0.44-0.38 (m, 2H), 0.17-0.14 (m, 2H).

### Example 15. 5-[5-[(1R)-1-(3,5-dichloro-4-pyridyl)ethoxy]-6-methoxy-1H-indazol-3-yl]-2-(3-methyl-3-pyrrolidin-1-yl-azetidin-1-yl)pyridine-3-carbonitrile

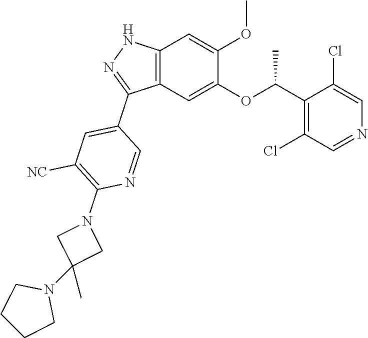 [SMILES: COc1cc2[nH]nc(-c3cnc(N4CC(C)(N5CCCC5)C4)c(C#N)c3)c2cc1O[C@H](C)c1c(Cl)cncc1Cl]

To a solution of [1-[3-cyano-5-[5-[(1R)-1-(3,5-dichloro-4-pyridyl)ethoxy]-6-methoxy-1-tetrahydropyran-2-yl-indazol-3-yl]-2-pyridyl]-3-methyl-azetidin-3-yl]methanesulfonate (300 mg, 0.44 mmol, 1.0 eq), DMAP (107 mg, 0.87 mmol, 2.0 eq) and pyrrolidine (621 mg, 8.76 mmol, 20.0 eq) in ACN (6 mL) was added t-BuONa (214 mg, 2.18 mmol, 5.0 eq). The reaction mixture was stirred for 6 h at 80° C. After the reaction was completed, the mixture was diluted with EtOAc (100 mL) and washed with brine (20 mL×5). The organic layer was dried over Na₂SO₄ and concentrated. The crude product was purified by prep-TLC (DCM/MeOH=20/1) to give a yellow solid (120 mg, 42% yield). LCMS m/z=662.1 (M+H).

Step 2. 5-[5-[(1R)-1-(3,5-Dichloro-4-pyridyl)ethoxy]-6-methoxy-1H-indazol-3-yl]-2-(3-methyl-3-pyrrolidin-1-yl-azetidin-1-yl)pyridine-3-carbonitrile. To a solution of product step 1 (120 mg, 0.18 mmol, 1.0 eq) in DCM (4 mL) was added TFA (2 mL). The solution was stirred at rt for 3 h. After the reaction completion, the mixture was concentrated in vacuum. The crude product was basified with a saturated NaHCO₃ solution, and extracted with EtOAc (30 mL×3). The combined organic layers were washed with brine (50 mL×2), dried over Na₂SO₄ and concentrated in vacuum. The residue was purified by Prep-HPLC (Prep-C18, 5 μM Triart column, 20×150 mm, YMC-Actus; gradient elution of 40% to 50% MeCN in water over a 15 min period (containing 0.05% NH₃·H₂O) to give a light-yellow solid (55 mg, 53% yield). LCMS m/z=578.3 (M+1); 1H NMR (400 MHz, DMSO-d6) δ 12.97 (brs, 1H), 8.71 (d, J=2.0 Hz, 1H), 8.59 (s, 2H), 8.07 (d, J=2.4 Hz, 1H), 7.04 (s, 1H), 7.00 (s, 1H), 6.00 (q, J=6.4 Hz, 1H), 4.31-4.29 (m, 2H), 4.00-3.98 (m, 2H), 3.86 (s, 3H), 2.66-2.58 (m, 4H), 1.76 (d, J=6.4 Hz, 3H), 1.74-1.68 (m, 4H), 1.45 (s, 3H).

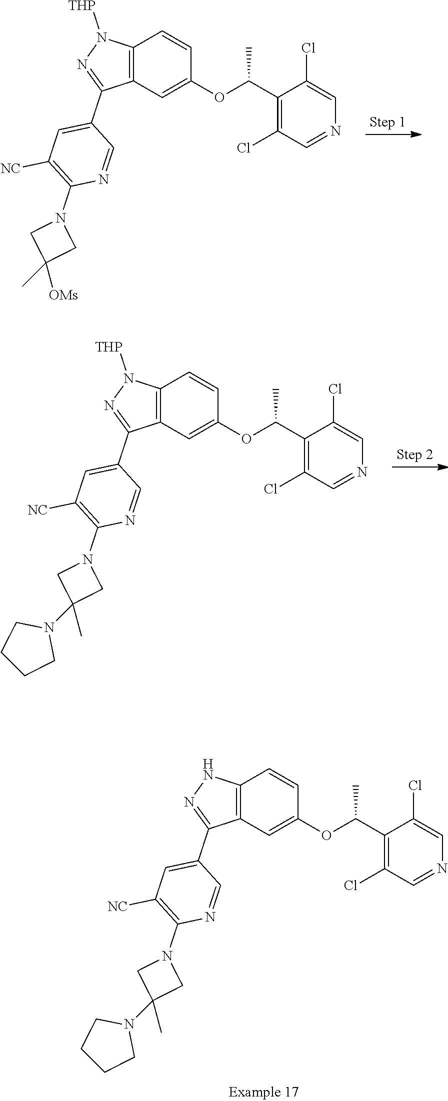

### Example 17. (R)-5-(5-(1-(3,5-Dichloropyridin-4-yl)ethoxy)-1H-indazol-3-yl)-2-(3-methyl-3-(pyrrolidin-1-yl)azetidin-1-yl)nicotinonitrile

Step 1. 5-[5-[(1R)-1-(3,5-dichloro-4-pyridyl)ethoxy]-1-tetrahydropyran-2-yl-indazol-3-yl]-2-(3-methyl-3-pyrrolidin-1-yl-azetidin-1-yl)pyridine-3-carbonitrile. To a solution of [1-[3-cyano-5-[5-[(1R)-1-(3,5-dichloro-4-pyridyl)ethoxy]-1-tetrahydropyran-2-yl-indazol-3-yl]-2-pyridyl]-3-methyl-azetidin-3-yl]methanesulfonate (250 mg, 0.38 mmol, 1.0 eq) and pyrrolidine (540 mg, 7.60 mmol, 20.0 eq) in ACN (250 mL) was added t-BuONa (182 mg, 1.90 mmol, 5.0 eq) at 25° C. The reaction mixture was stirred for 16 h at 80° C. After cooling to room temperature, the mixture was concentrated and the residue was diluted with DCM (100 mL). The organic layer was washed with water (50 mL×2) and brine (50 mL×2), dried over Na₂SO₄ and concentrated. The crude product was purified by silica gel column (DCM/MeOH=15/1) to give a yellow solid (160 mg, 66% yield). LCMS m/z=642.3 (M+H).

Step 2. (R)-5-(5-(1-(3,5-Dichloropyridin-4-yl)ethoxy)-1H-indazol-3-yl)-2-(3-methyl-3-(pyrrolidin-1-yl)azetidin-1-yl)nicotinonitrile. To a solution of product step 1 (160 mg, 0.25 mmol, 1.0 eq) in DCM (3 mL) was added TFA (1 mL). The reaction was stirred at rt for 4 h. After completion, the solution was concentrated under vacuum. The crude product was treated with DCM/MeOH (V:V=5:1, 20 mL), NaHCO₃ solid was added and stirred for 30 minutes. The solid was filtered out and filtrate was concentrated. The residue was purified by Prep-HPLC (Prep-C18, 5 μM Triart column, 20×150 mm, YMC-Actus; gradient elution of 40% MeCN in water to 60% MeCN in water containing 0.05% NH₃·H₂O over a 10 min period to give a yellow solid (47 mg, 34% yield). LCMS m/z=548.2 (M+H); 1H NMR (400 MHz, DMSO-d6) δ 13.17 (s, 1H), 8.76 (d, J=2.4 Hz, 1H), 8.57 (s, 2H), 8.14 (d, J=2.4 Hz, 1H), 7.48 (d, J=9.2 Hz, 1H), 7.16 (d, J=2.0 Hz, 1H), 7.11 (dd, J=8.8 Hz, 2.0 Hz, 1H), 6.14 (q, J=6.4 Hz, 1H), 4.30 (d, J=8.8 Hz, 2H), 3.99 (d, J=8.8 Hz, 2H), 2.69-2.59 (m, 4H), 1.79-1.70 (m, 7H), 1.45 (s, 3H).

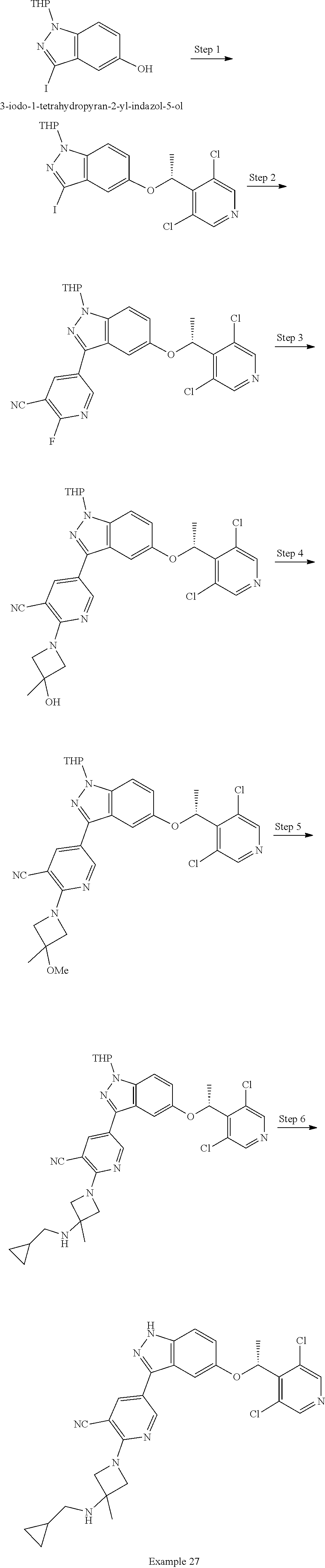

### Example 27. 2-[3-(Cyclopropylmethylamino)-3-methyl-azetidin-1-yl]-5-[5-[(1R)-1-(3,5-dichloro-4-pyridyl)ethoxy]-1H-indazol-3-yl]pyridine-3-carbonitrile

Step 1. 5-((R)-1-(3,5-Dichloropyridin-4-yl)ethoxy)-3-iodo-1-(tetrahydro-2H-pyran-2-yl)-1H-indazole. A mixture of 3-iodo-1-tetrahydropyran-2-yl-indazol-5-ol (1.0 g, 2.90 mmol, 1.0 equiv), [(1S)-1-(3,5-dichloro-4-pyridyl)ethyl]methanesulfonate (780 mg, 2.90 mmol, 1.0 equiv) and cesium carbonate (1.41 g, 14.45 mmol, 1.5 equiv) in N,N-dimethylformamide (20 mL) was heated at 130° C. for 16 h. The volatiles were removed under reduced pressure and the residue was suspended in saturated ammonium chloride (50 mL). The solution was extracted with ethyl acetate (3×50 mL). The combined organic layers were dried over sodium sulfate, filtered and concentrated under reduced pressure. The crude product was purified on a Buchi automated chromatography system (Sorbtech 40 g silica gel column), eluting with a gradient of 0 to 30% ethyl acetate in heptanes to give a white solid (1.01 g, 88% yield). LCMS m/z=517.2 (M+H).

Step 2. 5-[5-[(1R)-1-(3,5-Dichloro-4-pyridyl)ethoxy]-1-tetrahydropyran-2-yl-indazol-3-yl]-2-fluoro-pyridine-3-carbonitrile. To a solution of 5-((R)-1-(3,5-dichloropyridin-4-yl)ethoxy)-3-iodo-1-(tetrahydro-2H-pyran-2-yl)-1H-indazole (5.0 g, 9.65 mmol, 1.0 eq) and 2-fluoro-5-(4,4,5,5-tetramethyl-1,3,2-dioxaborolan-2-yl)pyridine-3-carbonitrile (3.11 g, 12.54 mmol, 1.3 eq) in dioxane (100 mL) and water (10 mL) was added K₂CO₃ (2.66 g, 19.30 mmol, 1.0 eq) and Pd(dppf)Cl₂ (705 mg, 0.965 mmol, 0.1 eq) at 25° C. The resulting mixture was stirred at 90° C. for 2 h under N₂. After cooling to rt, the mixture was quenched with water (50 mL), and extracted with ethyl acetate (50 mL×3). The combined organic phases were washed with brine (100 mL), dried over anhydrous Na2SO₄, filtered and concentrated. The crude product was purified by silica gel chromatography (Petroleum Ether/EtOAc=3/1) to give a white solid (4.8 g, 97% yield). LCMS m/z=512.2 (M+H).

Step 3. 5-[5-[(1R)-1-(3,5-Dichloro-4-pyridyl)ethoxy]-1-tetrahydropyran-2-yl-indazol-3-yl]-2-(3-hydroxy-3-methyl-azetidin-1-yl)pyridine-3-carbonitrile. To a solution of 5-[5-[(1R)-1-(3,5-dichloro-4-pyridyl)ethoxy]-1-tetrahydropyran-2-yl-indazol-3-yl]-2-fluoro-pyridine-3-carbonitrile (3.2 g, 6.25 mmol, 1.0 eq) and 3-methylazetidin-3-ol (1.54 g, 12.49 mmol, 2.0 eq) in DMSO (10 mL) was added DIEA (2.42 g, 18.74 mmol, 3.0 eq) at 25° C. The resulting solution was stirred at 90° C. for 3 h, cooled to room temperature, quenched with water (20 mL), and extracted with ethyl acetate (20 mL×3). The combined organic phases were washed with brine (30 mL), dried with anhydrous Na₂SO₄, filtered and concentrated. The crude product was purified by silica gel chromatography (Petroleum Ether/EtOAc=2/1) to give a white solid (3.1 g, 86% yield). LCMS m/z=579.3 (M+H).

Step 4. [1-[3-Cyano-5-[5-[(1R)-1-(3,5-dichloro-4-pyridyl)ethoxy]-1-tetrahydropyran-2-yl-indazol-3-yl]-2-pyridyl]-3-methyl-azetidin-3-yl]methanesulfonate. To a solution of product step 3 (3.1 g, 5.35 mmol, 1.0 eq) in DCM (50 mL) was added TEA (1.08 g, 10.7 mmol, 2.0 eq). Methanesulfonyl chloride (738 mg, 6.42 mmol, 1.2 eq) was added dropwise at 0° C. The resulting solution was stirred at rt for 2 h and was concentrated. The crude product was purified by silica gel chromatography (Petroleum Ether/EtOAc=3/1) to give a white solid (3.3 g, 94% yield). LCMS m/z=657.2 (M+1).

Step 5. 2-[3-(Cyclopropylmethylamino)-3-methyl-azetidin-1-yl]-5-[5-[(1R)-1-(3,5-dichloro-4-pyridyl)ethoxy]-1-tetrahydropyran-2-yl-indazol-3-yl]pyridine-3-carbonitrile. To a solution of product step 4 (250 mg, 0.381 mmol, 1.0 eq) and cyclopropylmethylamine (271 mg, 3.81 mmol, 10.0 eq) and in ACN (5 mL) was added t-BuONa (183 mg, 1.91 mmol, 5.0 eq) at 25° C. The resulting mixture was stirred at 90° C. for 12 h under N₂. After cooling to room temperature, the mixture was quenched with water (10 mL) and extracted with ethyl acetate (10 mL×3). The combined organic phases were dried over anhydrous Na₂SO₄, filtered and concentrated. The crude product was purified by silica gel chromatography (Petroleum Ether/EtOAc=1/1) to give a yellow solid (180 mg, 73% yield). LCMS m/z=632.3 (M+H).

Step 6. 2-[3-(Cyclopropylmethylamino)-3-methyl-azetidin-1-yl]-5-[5-[(1R)-1-(3,5-dichloro-4-pyridyl)ethoxy]-1H-indazol-3-yl]pyridine-3-carbonitrile. To a solution of product step 5 (180 mg, 0.285 mmol) in DCM (1 mL) was added TFA (0.5 mL) at rt and stirred for 3 h. After completion, the reaction was concentrated in vacuum. The crude product was treated with DCM/MeOH (5:1, 10 mL), solid NaHCO₃ was added to the solution and stirred for 20 minutes. The solid was filtered out and the filtrate was concentrated. The residue was purified by prep-TLC (Petroleum Ether/EtOAc=1/1) to give a yellow solid (54 mg, 35% yield). LCMS m/z=548.3 (M+H); 1H NMR (400 MHz, DMSO-d6) δ 13.17 (s, 1H), 8.76 (d, J=2.4 Hz, 1H), 8.57 (s, 2H), 8.12 (d, J=2.4 Hz, 1H), 7.48 (d, J=8.8 Hz, 1H), 7.15 (d, J=2.0 Hz, 1H), 7.11 (dd, J=8.8 Hz, 2.0 Hz, 1H), 6.14 (q, J=6.8 Hz, 1H), 4.14 (d, J=8.8 Hz, 2H), 4.03 (d, J=8.4 Hz, 2H), 2.41 (d, J=7.2 Hz, 2H), 1.76 (d, J=6.4 Hz, 3H), 1.42 (s, 3H), 0.90-0.83 (m, 1H), 0.44-0.38 (m, 2H), 0.17-0.13 (m, 2H).

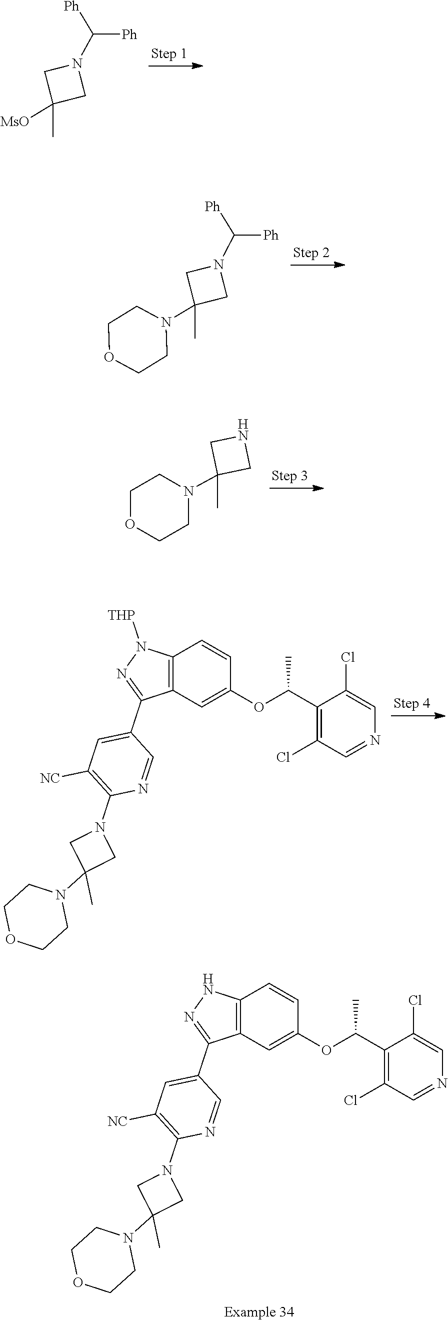

### Example 34

Step 1. 4-(1-benzhydryl-3-methyl-azetidin-3-yl)morpholine. To a solution of (1-benzhydryl-3-methyl-azetidin-3-yl) methanesulfonate (880 mg, 2.65 mmol, 1.0 eq) and morpholine (692 mg, 7.95 mmol, 3.0 eq) in MeCN (10 mL) was added Cs₂CO₃ (2.58 g, 7.95 mmol, 3.0 eq). The reaction mixture was stirred for 16 h at 80° C. The mixture was cooled, to rt, diluted with EtOAc (30 mL), washed with brine (20 mL×2) and concentrated. The crude product was purified by silica gel flash column chromatography (Petroleum ether/EtOAc=2/1) to give a yellow solid (680 mg, 79% yield). LCMS m/z=323.3 (M+H).

Step 2. 4-(3-Methylazetidin-3-yl)morpholine. To a solution of 4-(1-benzhydryl-3-methyl-azetidin-3-yl)morpholine (580 mg, 1.80 mmol, 1.0 eq) in MeOH (20 mL) was added Pd/C (580 mg, 1.0% wt). The suspension was degassed under vacuum and charged with hydrogen three times and stirred for 16 h at rt under H₂ balloon. After the reaction was completed, the solid was filtered off, the filtrate was concentrated to give a yellow oil (600 mg, crude). This material was used directly in the next step. LCMS m/z=157.3 (M+H).

Step 3. 5-[5-[(1R)-1-(3,5-dichloro-4-pyridyl)ethoxy]-1-tetrahydropyran-2-yl-indazol-3-yl]-2-(3-methyl-3-morpholino-azetidin-1-yl)pyridine-3-carbonitrile. To a solution of 5-[5-[(1R)-1-(3,5-dichloro-4-pyridyl)ethoxy]-1-tetrahydropyran-2-yl-indazol-3-yl]-2-fluoro-pyridine-3-carbonitrile (400 mg, 0.78 mmol, 1.0 eq) and 4-(3-methylazetidin-3-yl)morpholine (300 mg, crude) in DMSO (5.0 mL) was added DIEA (805 mg, 6.24 mmol, 8.0 eq). The reaction mixture was stirred for 2 h at 80° C. After the reaction was completed, the mixture was diluted with EtOAc (40 mL), washed with brine (30 mL×2) and was concentrated. The crude product was purified by silica gel flash column chromatography (DCM/MeOH=20/1) to give a yellow solid (320 mg, 63% yield). LCMS m/z=648.3 (M+H).

Step 4. 5-[5-[(1R)-1-(3,5-dichloro-4-pyridyl)ethoxy]-1H-indazol-3-yl]-2-(3-methyl-3-morpholino-azetidin-1-yl)pyridine-3-carbonitrile. To a solution of product step 3 (290 mg, 0.45 mmol, 1.0 eq) in DCM (5 mL) was added TFA (1 mL) and stirred at 25° C. for 3 h. After the reaction was completed, the reaction mixture was concentrated in vacuum. The crude product was adjusted pH to 7-8 by addition of the saturate NaHCO₃ solution and extracted with DCM (20 mL×3). The combined organic layers were washed with brine (50 mL), dried over Na₂SO₄ and concentrated. The crude product was purified by Prep-HPLC (Prep-C18, 5 μM Triart column, 20×150 mm, YMC-Actus; gradient elution of 35% MeCN in water to 60% MeCN in water with 0.05% NH₃·H₂O over a 11 min period,) to give a white solid (151 mg, 60% yield). LCMS m/z=564.3 (M+H); 1H NMR (400 MHz, DMSO-d6) δ 13.18 (s, 1H), 8.76 (d, J=2.4 Hz, 1H), 8.57 (s, 2H), 8.15 (d, J=1.6 Hz, 1H), 7.49 (d, J=8.8 Hz, 1H), 7.15 (s, 1H), 7.10 (dd, J=9.2 Hz, 2.0 Hz, 1H), 6.14 (q, J=6.4 Hz, 1H), 4.12 (d, J=8.8 Hz, 2H), 3.98 (d, J=8.4 Hz, 2H), 3.67-3.57 (m, 4H), 2.47-2.40 (m, 4H), 1.76 (d, J=6.4 Hz, 3H), 1.35 (s, 3H).

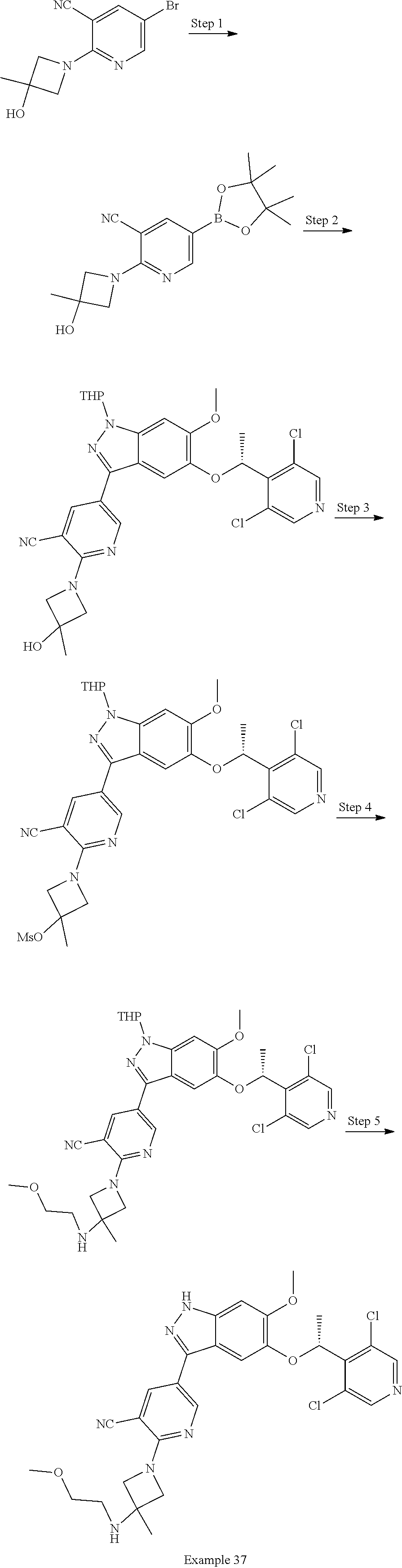

### Example 37. 5-[5-[(1R)-1-(3,5-dichloro-4-pyridyl)ethoxy]-6-methoxy-1H-indazol-3-yl]-2-[3-(2-methoxyethylamino)-3-methyl-azetidin-1-yl]pyridine-3-carbonitrile

Step 1. 2-(3-Hydroxy-3-methylazetidin-1-yl)-5-(4,4,5,5-tetramethyl-1,3,2-dioxaborolan-2-yl)nicotinonitrile. A mixture of 5-bromo-2-(3-hydroxy-3-methyl-azetidin-1-yl)pyridine-3-carbonitrile (1.724 g, 6.43 mmol, 1 equiv), bis(pinacolato)diboron (2.45 g, 9.645 mmol, 1.5 equiv), potassium acetate (1.26 g, 12.86 mmol, 2 equiv) and [1,1′-bis(diphenylphosphino)ferrocene]-dichloropalladium(II) (0.47 mg, 0.643 mmol, 0.1 equiv) in 1,4-dioxane (10 mL) was sparged with nitrogen for 15 minutes then heated at 90° C. for 17 hours. LCMS analysis indicated that the reaction was complete and was used subsequently in next step. LCMS m/z=234 (boronic acid) and 316 (M+H).

Step 2. 5-(5-((R)-1-(3,5-Dichloropyridin-4-yl)ethoxy)-6-methoxy-1-(tetrahydro-2H-pyran-2-yl)-1H-indazol-3-yl)-2-(3-hydroxy-3-methylazetidin-1-yl)nicotinonitrile. The mixture of reaction step 1 (6.43 mmol, 1.5 equiv) in 1,4-dioxane was treated with 5-[(1R)-1-(3,5-dichloro-4-pyridyl)ethoxy]-3-iodo-6-methoxy-1-tetrahydropyran-2-yl-indazole (2.35 g, 4.287 mmol, 1 equiv), [1,1′-bis(diphenylphosphino)ferrocene]dichloropalladium(II) (314 mg, 0.429 mmol, 0.1 equiv) and potassium carbonate (1.18 g, 8.573 mmol, 2 eq) and water (7.0 mL). After sparging with nitrogen for 10 minutes the reaction was heated at 90° C. for 20 hours. The mixture was cooled to room temperature and diluted with ethyl acetate (100 mL) and water (30 mL). The layers were separated, and the aqueous layer was extracted with ethyl acetate (3×30 mL). The combined organic layers were washed with saturated brine (50 mL), dried over sodium sulfate, filtered and concentrated under reduced pressure. The crude product was purified on an Interchim automated chromatography system (Sorbtech 120 g silica gel column), eluting with a gradient of 0 to 100% ethyl acetate in heptanes to give a brown solid (1.77 g, 68% yield for 2 steps). LCMS m/z=610 (M+H).

Step 3. 1-(3-Cyano-5-(5-((R)-1-(3,5-dichloropyridin-4-yl)ethoxy)-6-methoxy-1-(tetrahydro-2H-pyran-2-yl)-1H-indazol-3-yl)pyridin-2-yl)-3-methylazetidin-3-yl methanesulfonate. N,N-Diisopropylethylamine (1.52 mL, 8.712 mmol, 23 equiv) was added to a suspension of product step 2 (1.77 g, 2.904 mmol, 1 equiv) in anhydrous dichloromethane (120 mL) at room temperature. After stirring at room temperature for 30 minutes, methanesulfonic anhydride (1.01 g, 5.808 mmol, 2 equiv) was added and the mixture was stirred at room temperature for 2.5 hours. LCMS analysis indicated that the reaction was complete. Water (50 mL) was added and the layers were separated. The aqueous layer was extracted with dichloromethane (3×50 mL). The combined organic layers were washed with saturated brine (50 mL), dried over sodium sulfate, filtered and concentrated under reduced pressure. The product was purified on an Interchim automated chromatography system (Sorbtech 120 g silica gel column), eluting with a gradient of 0 to 100% ethyl acetate in heptanes to give a white foam (1.40 g, 70% yield). LCMS m/z=687 (M+H).

Step 4. 5-(5-((R)-1-(3,5-Dichloropyridin-4-yl)ethoxy)-6-methoxy-1-(tetrahydro-2H-pyran-2-yl)-1H-indazol-3-yl)-2-(3-((2-methoxyethyl)amino)-3-methylazetidin-1-yl)nicotinonitrile. A mixture of product step 3 (299 mg, 0.44 mmol, 1.0 equiv), 2-methoxyethan-1-amine (163 mg, 2.2 mmol, 5 equiv) and sodium tert-butoxide (125 mg, 1.53 mmol, 3 equiv) in acetonitrile (5 mL) was heated at 60° C. for 16 hours. After cooling to room temperature, the mixture was diluted with water (1 mL) and concentrated under reduced pressure. The residue was redissolved in dimethyl sulfoxide (10 mL) and purified on a Biotage automated chromatography system (RediSep Rf GOLD 100 g HP C₁₈ column), eluting with a gradient of 0 to 100% acetonitrile in water to give an off-white solid (155 mg, 53% yield). LCMS m/z=666.3 (M+H).

Step 5. (R)-5-(5-(1-(3,5-Dichloropyridin-4-yl)ethoxy)-6-methoxy-1H-indazol-3-yl)-2-(3-((2-methoxyethyl)amino)-3-methylazetidin-1-yl)nicotinonitrile. 5-(5-((R)-1-(3,5-Dichloropyridin-4-yl)ethoxy)-6-methoxy-1-(tetrahydro-2H-pyran-2-yl)-1H-indazol-3-yl)-2-(3-((2-methoxyethyl)amino)-3-methylazetidin-1-yl)nicotinonitrile (155 mg, 0.23 mmol, 1.0 equiv) was treated with a 1 to 1 mixture of TFA in dichloromethane (4 mL) at room temperature for 3 hours. The mixture was concentrated under reduced pressure. The residue was dissolved in ethyl acetate (15 mL) and washed with saturated sodium carbonate (10 mL). The organic layer was dried over sodium sulfate, filtered and concentrated under reduced pressure. The residue was redissolved in dimethyl sulfoxide (10 mL) and purified on Biotage automated chromatography system (RediSep Rf GOLD 100 g HP C18 column), eluting with a gradient of 0 to 100% acetonitrile in water to give an off-white solid (56 mg, 42% yield) after lyophilization of product. LCMS m/z=582.2 (M+H); 1H NMR (400 MHz, DMSO-d6) δ=12.96 (br s, 1H), 8.71 (d, J=2.3 Hz, 1H), 8.59 (s, 2H), 8.05 (d, J=2.3 Hz, 1H), 7.04 (s, 1H), 7.01 (s, 1H), 5.99 (q, J=6.7 Hz, 1H), 4.11 (d, J=8.8 Hz, 2H), 4.03 (d, J=8.7 Hz, 2H), 3.86 (s, 3H), 3.44-3.34 (m, 2H), 3.28-3.25 (m, 3H), 2.70 (q, J=6.1 Hz, 2H), 2.48-2.27 (m, 1H), 1.76 (d, J=6.6 Hz, 3H), 1.45-1.36 (m, 3H).

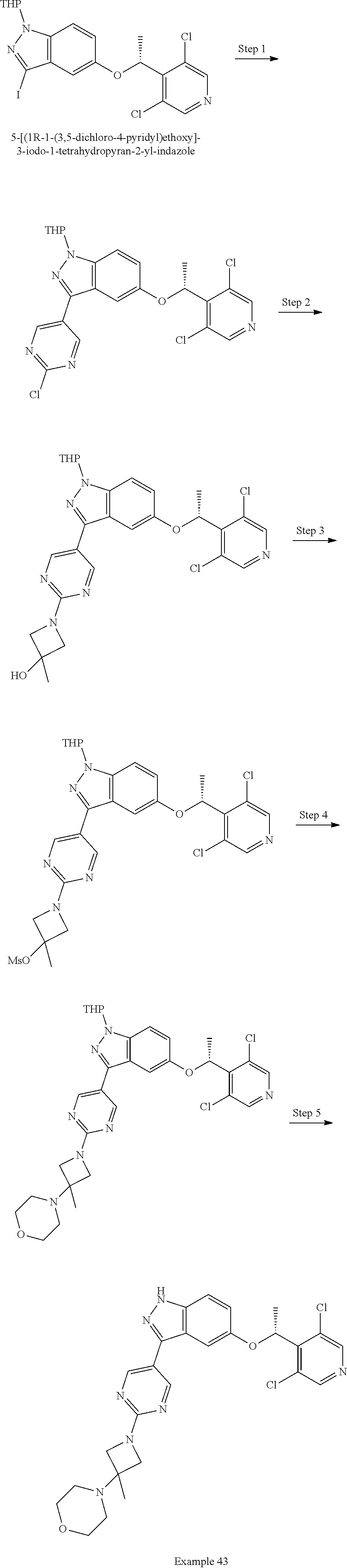

### Example 43. (R)-4-(1-(5-(5-(1-(3,5-dichloropyridin-4-yl)ethoxy)-1H-indazol-3-yl)pyrimidin-2-yl)-3-methylazetidin-3-yl)morpholine

Step 1. 3-(2-chloropyrimidin-5-yl)-5-[(1R)-1-(3,5-dichloro-4-pyridyl)ethoxy]-1-tetrahydropyran-2-yl-indazole. To a solution of 5-[(1R)-1-(3,5-dichloro-4-pyridyl)ethoxy]-3-iodo-1-tetrahydropyran-2-yl-indazole (4.0 g, 7.72 mmol, 1.0 eq) in dioxane (100 mL) and H₂O (10 mL) was added 2-chloro-5-(4,4,5,5-tetramethyl-1,3,2-dioxaborolan-2-yl)pyrimidine (3.71 g, 15.44 mmol, 2.0 eq), K₂C₃ (3.2 g, 23.16 mmol, 3.0 eq) and Pd(dppf)Cl₂ (565 mg, 0.772 mmol, 0.1 eq) at 25° C. The resulting mixture was stirred at 90° C. for 3 h under N₂. After cooling to room temperature, the mixture was concentrated under vacuum. The crude product was purified by silica gel chromatography (Petroleum Ether/EtOAc=2/1) to give a yellow solid (3.5 g, 90% yield). LCMS m/z=504.2 (M+H).

Step 2. 1-[5-[5-[(1R)-1-(3,5-dichloro-4-pyridyl)ethoxy]-1-tetrahydropyran-2-yl-indazol-3-yl]pyrimidin-2-yl]-3-methyl-azetidin-3-ol. To a solution of product step 1 (3.2 g, 6.34 mmol, 1.0 eq) in DMSO (30 mL) was added 3-methylazetidin-3-ol (940 mg, 7.61 mmol, 1.2 eq) and DIEA (2.45 g, 19.02 mmol, 3.0 eq), the mixture was stirred at 100° C. for 4 h under N₂. After cooling to room temperature, the reaction mixture was diluted with EtOAc (200 mL), and washed with water (100 mL×2). The organic layer was dried over Na₂SO₄, filtered and concentrated under reduced pressure to give light yellow oil (2.7 g). This material was used directly in the next step. LCMS m/z=555.3 (M+H).

Step 3. [1-[5-[5-[(1R)-1-(3,5-dichloro-4-pyridyl)ethoxy]-1-tetrahydropyran-2-yl-indazol-3-yl]pyrimidin-2-yl]-3-methyl-azetidin-3-yl]methanesulfonate. To a solution of product step 2 (2.7 g, crude) in DCM (100 mL) was added TEA (984 mg, 9.72 mmol, 2.0 eq) and MsCl (7.29 mmol, 832 mg, 1.5 eq) at 0° C. The resulting mixture was stirred at rt for 2 h. After completion, the reaction was concentrated under vacuum. The crude product was purified by silica gel chromatography (Petroleum Ether/EtOAc=3/1) to give a yellow solid (2.5 g, 63% yield for two steps). LCMS m/z=633.2 (M+H).

Step 4. 4-[1-[5-[5-[(1R)-1-(3,5-dichloro-4-pyridyl)ethoxy]-1-tetrahydropyran-2-yl-indazol-3-yl]pyrimidin-2-yl]-3-methyl-azetidin-3-yl]morpholine To a solution of product step 3 (200 mg, 0.316 mol, 1.0 eq) in ACN (2 mL) was added morpholine (138 mg, 1.58 mmol, 5.0 eq) and t-BuONa (177 mg, 1.58 mmol, 5.0 eq). The mixture was stirred at 90° C. for 12 h. After cooled to room temperature, the reaction mixture was concentrated under vacuum. The crude product was purified by silica gel chromatography (Petroleum Ether/EtOAc=1/1) to give a yellow solid (150 mg, 76% yield). LCMS m/z=624.3 (M+H).

Step 5. (R)-4-(1-(5-(5-(1-(3,5-dichloropyridin-4-yl)ethoxy)-1H-indazol-3-yl)pyrimidin-2-yl)-3-methylazetidin-3-yl)morpholine. To a solution of product step 4 (150 mg, 0.24 mmol, 1.0 eq) in DCM (3 mL) was added TFA (1.5 mL), the mixture was stirred at rt. for 3 h. After completion, the reaction mixture was concentrated in vacuum. The crude product was treated with DCM/MeOH (V:V=5:1, 10 mL), Solid NaHCO₃ to pH 8 was added to the solution and stirred for 20 minutes. The solid was filtered out and the filtrate was concentrated. The residue was purified by Prep-HPLC (Prep-C18, 5 μM Triart column, 20×150 mm, YMC-Actus; gradient elution of 35% MeCN in water to 65% MeCN in water containing 0.05% NH₃·H₂O over a 10 min period to give (36 mg, 28% yield) as a white solid. LCMS m/z=540.3 (M+H); 1H NMR (400 MHz, DMSO-d6) δ 13.13 (brs, 1H), 8.78 (s, 2H), 8.57 (s, 2H), 7.47 (d, J=9.2 Hz, 1H), 7.23 (d, J=2.0 Hz, 1H), 7.10 (dd, J=8.8 Hz, 2.4 Hz, 1H), 6.15 (q, J=6.4 Hz, 1H), 3.94 (d, J=8.4 Hz, 2H), 3.76 (d, J=8.4 Hz, 2H), 3.61 (t, J=4.0 Hz, 4H), 2.44 (t, J=4.0 Hz, 4H), 1.75 (d, J=6.4 Hz, 3H), 1.32 (s, 3H).

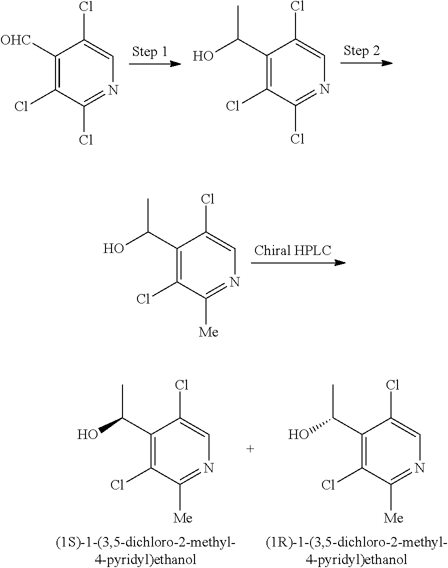

### (R)-1-(3,5-dichloro-2-methyl-4-pyridyl)ethanol and (S)-1-(3,5-dichloro-2-methyl-4-pyridyl)ethanol

Step 1. (Trichloro-4-pyridyl)ethanol. A 500 mL three-neck round bottom flask was charged with 2,3,5-trichloropyridine-4-carbaldehyde (40 g, 0.19 mol) and THE (200 mL). MeMgBr (70 mL, 0.21 mol) was added in portions and the mixture was stirred at −70° C. for 1 h. The reaction was quenched with aqueous ammonium chloride solution, extracted with ethyl acetate (200 mL×3), dried over Na₂SO₄ and concentrated. The residue was purified by silica gel column (PE/EA=30/1) to give (33 g) a yellow liquid (33 g, 77%). LCMS m/z=227 (M+H).

Step 2. 1-(3,5-Dichloro-2-methyl-4-pyridyl)ethanol. A mixture of product step 1 (33 g, 0.147 mmol), methylboronic acid (26.3 g, 0.429 mmol), K₂CO₃ (40 g, 0.290 mmol) and Pd(PPh₃)₂Cl₂ (3 g) in dioxane (300 mL) was stirred at 110° C. overnight. The resulting mixture was filtered and the filtrate was concentrated in vacuo to give the crude product, which was further purified by silica gel column chromatography to give a yellow liquid (15 g, 50%). LCMS m/z=206.1 (M+H). The product was separated by Prep-HPLC (Chiralpak ID 5×25 cm, hexanes/ethanol (80/20), 60 mL/min. 38° C.) to give (S)-1-(3,5-dichloro-2-methyl-4-pyridyl)ethanol (5 g) and (R)-1-(3,5-dichloro-2-methyl-4-pyridyl)ethanol (5 g) as yellow liquid. Peak 1 5.5 min; (S)-1-(3,5-dichloro-2-methyl-4-pyridyl)ethanol. LCMS m/z=206.1 (M+H). 1H NMR (400 MHz, CDCl3) δ 8.36 (s, 1H), 5.57 (m, 1H), 2.93 (b, 1H), 2.64 (s, 3H), 1.65 (d, 3H). Peak 2 6.9 min; (R)-1-(3,5-dichloro-2-methyl-4-pyridyl)ethanol. LCMS m/z=206.1 (M+H). 1H NMR (400 MHz, CDCl3) δ 8.36 (s, 1H), 5.57 (m, 1H), 2.93 (b, 1H), 2.64 (s, 3H), 1.65 (d, 3H).

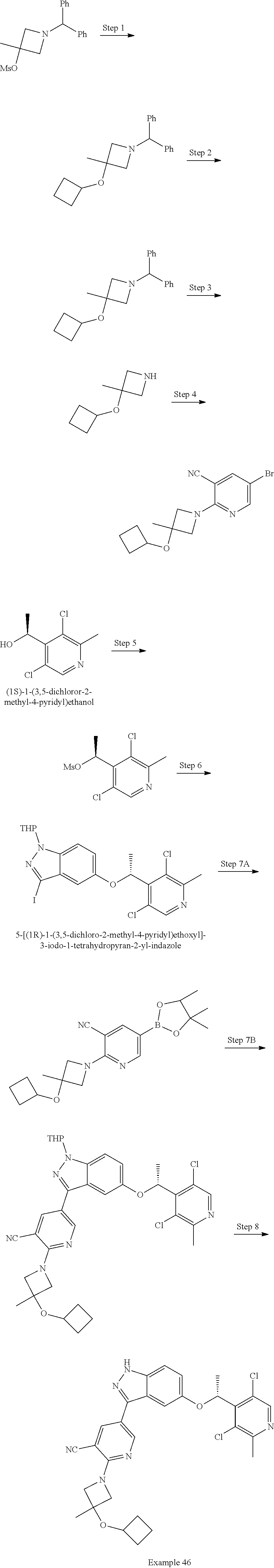

### Example 46

Step 1. 1-Benzhydryl-3-cyclobutoxy-3-methylazetidine. In a sealed tube, a solution of 1-benzhydryl-3-methylazetidin-3-yl methanesulfonate (3.0 g, 9.06 mmol, 1.0 equiv) in 1,4-dioxane (20 mL) was treated with cyclobutanol (1.3 g, 18.12 mmol, 2.0 equiv) and N,N-diisopropylethylamine (4.7 mL, 27.18 mmol, 3.0 equiv) at 100° C. After stirring for 4 days the solution was cooled to room temperature and concentrated under reduced pressure. The residue was diluted with ethyl acetate (20 mL) and washed with water (20 mL). The organic layer was concentrated under reduced pressure to give a light brown oil (500 mg, 18%). LCMS m/z=307.2 (M+H).

Step 2. 1-Benzhydryl-3-cyclobutoxy-3-methylazetidine hydrochloride. A solution of 1-benzhydryl-3-cyclobutoxy-3-methylazetidine (1.2 g, 3.9 mmol, 1.0 equiv) in 1,4-dioxane (10 mL) was treated with 4M HCl in 1,4-dioxane (3.9 mL, 15.63 mmol, 4.0 equiv) at room temperature for 16 hours. The mixture was concentrated under reduced pressure to give a light brown oil (505 mg, 37% yield). LCMS m/z=307.2 (free base M+H).

Step 3. 3-Cyclobutoxy-3-methylazetidine hydrochloride. A mixture of 1-benzhydryl-3-cyclobutoxy-3-methylazetidine hydrochloride (1.19 g, 3.46 mmol, 1.0 equiv) and 20% palladium hydroxide on carbon (0.24 g, 50% wet) in methanol (100 mL) was hydrogenated at 50 psi at room temperature for 16 hours. The mixture was filtered through Celite and the filtrate was concentrated under reduced pressure to give a dark brown oil (1.0 g, 62% yield). LCMS m/z=141.2 (free base, M+H).

Step 4. 5-Bromo-2-(3-cyclobutoxy-3-methylazetidin-1-yl)nicotinonitrile. A solution of 5-bromo-2-chloro-3-cyano-pyridine (633 mg, 2.98 mmol, 1.0 equiv) in acetonitrile (10 mL) was treated with 3-cyclobutoxy-3-methylazetidine hydrochloride (626 mg, 3.58 mmol, 1.2 equiv) and N,N-diisopropylethylamine (1.1 mL, 5.96 mmol, 2.0 equiv) at 60° C. for 16 hours. The crude mixture was cooled to room temperature and volatiles removed under reduced pressure. The residue was suspended in ethyl acetate (50 mL) and washed with saturated sodium bicarbonate (20 mL). The organic layer was dried over sodium sulfate, filtered and evaporated to dryness under reduced pressure. The residue was purified on a Buchi automated chromatography system (Sorbtech 40 g column), eluting with a gradient of 0 to 50% ethyl acetate in heptanes. Product containing fractions were pooled together, concentrated to dryness under reduced pressure and the residue was dried under vacuum at 50° C. for 16 hours to give a light brown oil (344 mg, 36% yield). LCMS m/z=322.2 (M+H).

Step 5. (S)-1-(3,5-Dichloro-2-methylpyridin-4-yl)ethyl methanesulfonate. Methanesulfonyl chloride (1.35 mL, 17.5 mmol, 1.2 equiv) was added to a solution of compound (S)-263-1 (3 g, 14.6 mmol, 1 equiv) and triethylamine (4 mL, 29.1 mmol, 2 equiv) in anhydrous dichloromethane (30 mL) at −10° C. After two hours, the reaction was warmed to room temperature and diluted with deionized water (30 mL, 0° C.). The layers were separated and the aqueous layer was extracted with dichloromethane (3×15 mL). The combined organic layers were dried over sodium sulfate, filtered and concentrated under reduced pressure. The crude yellow oil was absorbed onto Celite (20 g) and was purified on an Interchim automated chromatography system (Sorbtech, 100 g silica gel column) eluting with a gradient of 0 to 100% methyl tert-butyl ether and heptanes to give compound a white solid (4.04 g, 96%). LCMS m/z=284.1 (M+H).

Step 6. 5-((R)-1-(3,5-Dichloro-2-methylpyridin-4-yl)ethoxy)-3-iodo-1-(tetrahydro-2H-pyran-2-yl)-1H-indazole. A mixture of compound 3-iodo-1-tetrahydropyran-2-yl-indazol-5-ol (5.38 g, 15.6 mmol, 1.1 equiv), (S)-1-(3,5-dichloro-2-methylpyridin-4-yl)ethyl methanesulfonate (4.04 g, 14.2 mmol, 1 equiv) and cesium carbonate (9.27 g, 28.5 mmol, 2 equiv) in acetonitrile (80 mL) was heated at 80° C. overnight. The reaction was cooled to room temperature and was diluted with ethyl acetate (100 mL) and saturated brine solution (100 mL). The layers were separated and the aqueous layer was extracted with ethyl acetate (3×30 mL). The combined organic layers were dried over sodium sulfate, filtered and concentrated under reduced pressure. The residue was absorbed onto Celite (30 g) and was purified on an Interchim automated chromatography system (Sorbtech, 80 g silica gel column), eluting with a gradient of 0 to 100% ethyl acetate in heptanes to give a white solid (6.5 g, 86%). LCMS m/z=532.1 (M+H).

Step 7. 2-(3-Cyclobutoxy-3-methylazetidin-1-yl)-5-(5-((R)-1-(3,5-dichloro-2-methyl-pyridin-4-yl)ethoxy)-1-(tetrahydro-2H-pyran-2-yl)-1H-indazol-3-yl)nicotinonitrile. A mixture of 5-bromo-2-(3-cyclobutoxy-3-methylazetidin-1-yl)nicotinonitrile (344 mg, 1.06 mmol, 1.0 equiv), bis(pinacolato)diboron (400 mg, 1.6 mmol, 1.5 equiv), [1,1′-bis(diphenylphosphino)ferrocene]palladium (II) dichloride (77 mg, 0.11 mmol, 0.1 equiv) and potassium acetate (207 mg, 2.12 mmol, 2.0 equiv) in 1,4-dioxane (20 mL) was sparged with nitrogen for 10 minutes followed by heating at 90° C. for 32 hours. LC/MS analysis indicated complete conversion. The crude reaction mixture was cooled to room temperature and treated with 5-((R)-1-(3,5-dichloro-2-methylpyridin-4-yl)ethoxy)-3-iodo-1-(tetrahydro-2H-pyran-2-yl)-1H-indazole (473 mg, 0.89 mmol, 1.0 equiv), [1,1′-bis (diphenylphosphino)ferrocene]palladium (II) dichloride (65 mg, 0.09 mmol, 0.1 equiv), potassium carbonate (245 mg, 1.78 mmol, 2.0 equiv) and water (1.0 mL). The crude mixture was sparged with nitrogen for 10 minutes followed by heating at 100° C. for 16 hours. The crude mixture was cooled to room temperature and filtered through a pad of Celite. The filtrate was concentrated to dryness under reduced pressure. The residue was diluted with ethyl acetate (30 mL) and washed with water (20 mL). The organic layer was concentrated under reduced pressure. The residue was purified on a Buchi automated chromatography system (Sorbtech 80 g column), eluting with a gradient of 0 to 20% ethyl acetate in heptanes. Product containing fractions were pooled together, concentrated to dryness under reduced pressure and the resulting residue was dried under vacuum at 50° C. for 16 hours to give compound 585-3 (149 mg, 25% yield) as a light brown solid. LCMS m/z=650.1 (M+H).

Step 8. (R)-2-(3-Cyclobutoxy-3-methylazetidin-1-yl)-5-(5-(1-(3,5-dichloro-2-methyl-pyridin-4-yl)ethoxy)-1H-indazol-3-yl)nicotinonitrile. 2-(3-Cyclobutoxy-3-methylazetidin-1-yl)-5-(5-((R)-1-(3,5-dichloropyridin-4-yl)ethoxy)-1-(tetrahydro-2H-pyran-2-yl)-1H-indazol-3-yl)nicotinonitrile (147 mg, 0.22 mmol, 1.0 equiv) was treated with a 1 to 1 mixture of dichloromethane (1.5 mL) and trifluoroacetic acid (1.5 mL, 19.6 mmol, 89.1 equiv) at room temperature for 6 hours. The volatiles were removed under reduced pressure. The residue was diluted with dichloromethane (5 mL) then treated with water (2 mL) and sodium bicarbonate (500 mg). The layers were separated and the organic layer was concentrated under reduced pressure onto Celite (10 g). The material was purified on a Buchi automated chromatography system (RediSep C_{18 100} g column), eluting with a gradient of 0 to 80% acetonitrile in water. Product containing fractions were pooled together, concentrated under reduced pressure and the resulting residue dried under vacuum at 50° C. for 16 hours to give an off white solid (35 mg, 27% yield) LCMS m/z=563.2 (M+H); 1H NMR (400 MHz, DMSO-d6) δ=13.18 (s, 1H), 8.70 (d, J=2.2 Hz, 1H), 8.41 (s, 1H), 8.16 (d, J=2.2 Hz, 1H), 7.49 (d, J=8.9 Hz, 1H), 7.11 (dd, J=2.3, 9.0 Hz, 1H), 7.07 (s, 1H), 6.10 (q, J=6.7 Hz, 1H), 4.20-3.98 (m, 5H), 2.58-2.53 (m, 3H), 2.26-2.10 (m, 2H), 2.02-1.84 (m, 2H), 1.75 (d, J=6.7 Hz, 3H), 1.61 (q, J=10.0 Hz, 1H), 1.50 (s, 3H), 1.49-1.39 (m, 1H).

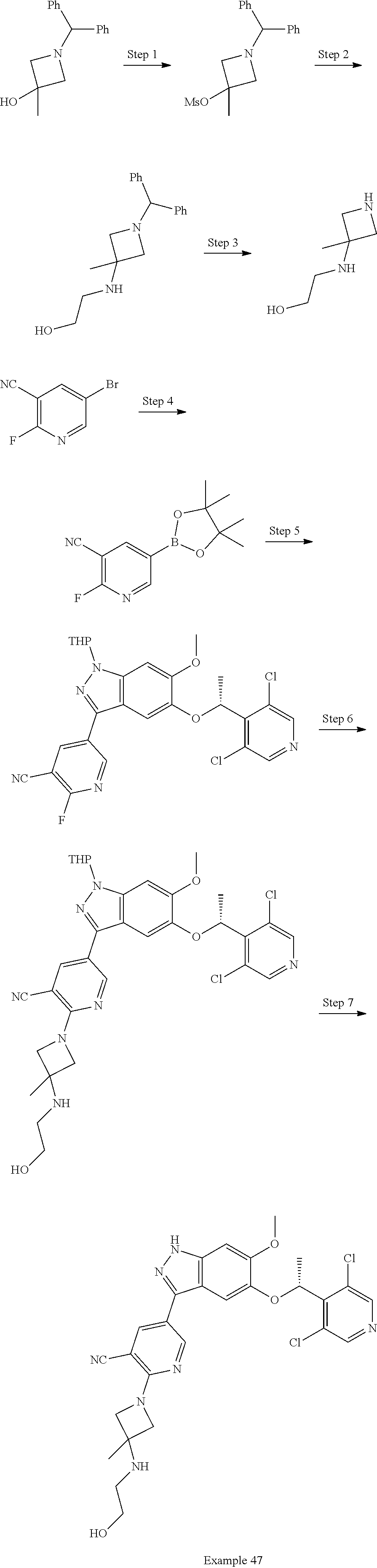

### Example 47. 5-[5-[(1R)-1-(3,5-dichloro-4-pyridyl)ethoxy]-6-methoxy-1H-indazol-3-yl]-2-[3-(2-hydroxyethylamino)-3-methyl-azetidin-1-yl]pyridine-3-carbonitrile

Step 1. (1-Benzhydryl-3-methyl-azetidin-3-yl) methanesulfonate. To a solution of 1-benzhydryl-3-methyl-azetidin-3-ol (5.0 g, 19.7 mmol, 1.0 eq) in DCM (50 mL) was added TEA (6.0 g, 59.2 mmol, 3.0 eq), and then MsCl (3.2 g, 27.6 mmol, 1.4 eq) dropwise added at 0° C. The reaction mixture was stirred for 3 h at rt. After the reaction was completed, the mixture was diluted with DCM (50 mL) and washed with brine (30 mL×3). The organic layer was dried over Na₂SO₄ and concentrated. The crude product was purified by silica gel flash column chromatography (Petroleum Ether/EtOAc=3/1) to give a white solid (3.5 g, 54% yield). LCMS m/z=332.3 (M+H).

Step 2. 2-[(1-Benzhydryl-3-methyl-azetidin-3-yl)amino]ethanol. To a solution of (1-benzhydryl-3-methyl-azetidin-3-yl) methanesulfonate (1.5 g, 4.5 mmol, 1.0 eq) in CH₃CN (15 mL) was added 2-aminoethanol (1.4 g, 22.6 mmol, 5.0 eq) and Cs₂CO₃ (4.5 g, 13.6 mmol, 3.0 eq) at rt. The reaction mixture was stirred for 16 h at 80° C. After the reaction was completed, the solid was filtered off, the filtrate was concentrated, and the crude product was purified by silica gel flash column chromatography (DCM/MeOH=20/1) to give a white solid (1.1 g, 82% yield). LCMS m/e=297.3 (M+H).

Step 3. 2-[(3-Methylazetidin-3-yl)amino]ethanol. To a solution of 2-[(1-benzhydryl-3-methyl-azetidin-3-yl)amino]ethanol (1.0 g, 3.4 mmol, 1.0 eq) in MeOH (10 mL) was added Pd/C (1.23 g, 10% wt) in portions under N₂. The suspension was degassed under vacuum and charged with hydrogen three times. The mixture was stirred for 16 h at rt under H₂ balloon. The solid was filtered off and the filtrate was concentrated to give colorless oil. LCMS m/e=131.3 (M+H).

Step 4. 2-Fluoro-5-(4,4,5,5-tetramethyl-1,3,2-dioxaborolan-2-yl)pyridine-3-carbonitrile. To a solution of 5-bromo-2-fluoro-pyridine-3-carbonitrile (10 g, 49.8 mmol, 1.0 eq) and bis(pinacolato)diboron (25.3 g, 99.5 mmol, 2.0 eq) in dioxane (200 mL) was added KOAc (20.6 g, 149.3 mmol, 3.0 eq) and Pd(dppf)Cl₂ (2.0 g, 20 wt %). The reaction mixture was stirred for 5 h at 90° C. under N₂ protection. After the reaction was completed, the solid was filtered out and the filtrate was concentrated in vacuum. The crude product was purified by silica gel column (Petroleum Ether/EtOAc=10/1) to give a white solid (12 g, 59% yield). LCMS m/z=249.2 (M+H); 1H NMR (400 MHz, DMSO-d6) δ 8.68 (d, J=1.6 Hz, 1H), 8.63 (dd, J=9.2 Hz, 1.6 Hz, 1H), 1.32 (s, 12H).

Step 5. 5-[5-[(1R)-1-(3,5-dichloro-4-pyridyl)ethoxy]-6-methoxy-1-tetrahydropyran-2-yl-indazol-3-yl]-2-fluoro-pyridine-3-carbonitrile. To a solution of 2-fluoro-5-(4,4,5,5-tetramethyl-1,3,2-dioxaborolan-2-yl)pyridine-3-carbonitrile (1.0 g, 4.0 mmol, 2.2 eq) and 5-[(1R)-1-(3,5-dichloro-4-pyridyl)ethoxy]-3-iodo-6-methoxy-1-tetrahydropyran-2-yl-indazole (1.0 g, 1.8 mmol, 1.0 eq) in dioxane (20 mL) and H₂O (2 mL) was added K₂CO₃ (745 mg, 5.4 mmol, 3.0 eq) and Pd(dppf)Cl₂ (100 mg, 10% wt) at rt. The mixture was stirred for 2 h at 90° C. under N₂ protection. After the reaction was completed, the solid was filtered out and the filtrate was concentrated in vacuum. The crude product was purified by silica gel column (Petroleum Ether/EtOAc=1/1) to give an off-white solid (900 mg, 91% yield). LCMS m/z=542.2 (M+H).

Step 6. 5-[5-[(1R)-1-(3,5-Dichloro-4-pyridyl)ethoxy]-6-methoxy-1-tetrahydropyran-2-yl-indazol-3-yl]-2-[3-(2-hydroxyethylamino)-3-methyl-azetidin-1-yl]pyridine-3-carbonitrile. To a solution of product step 4 (150 mg, 0.28 mmol, 1.0 eq) and 2-[(3-methylazetidin-3-yl)amino]ethanol (54 mg, 0.41 mmol, 1.5 eq) in DMSO (2 mL) was added DIEA (178 mg, 1.38 mmol, 5.0 eq). The reaction mixture was stirred for 16 h at 90° C. After the reaction was completed, the mixture was diluted with EtOAc (30 mL) and washed with brine (20 mL×3). The organic layer was dried over Na₂SO₄ and concentrated. The crude product was purified by prep-TLC (DCM/MeOH=12/1) to give a white solid (120 mg, 67% yield). LCMS m/z=652.3 (M+H).

Step 7. 5-[5-[(1R)-1-(3,5-Dichloro-4-pyridyl)ethoxy]-6-methoxy-1H-indazol-3-yl]-2-[3-(2-hydroxyethylamino)-3-methyl-azetidin-1-yl]pyridine-3-carbonitrile. To a solution of product step 5 (110 mg, 0.17 mmol, 1.0 eq) in DCM (6 mL) was added TFA (2 mL). The reaction was stirred at rt for 5 h and was concentrated in vacuum. The crude product was treated with DCM/MeOH (15 mL, V:V=5/1), then NaHCO₃ (excess) was added to the solution and stirred for 20 minutes at rt., and then DCM (20 mL) was added. The solid was filtered off and the filtrate was concentrated. The residue was purified by Prep-TLC (DCM/MeOH=7/1) to give a white solid (54 mg, 57% yield). LCMS m/z=568.3 (M+H); 1H NMR (400 MHz, DMSO-d6) δ12.98 (s, 1H), 8.73 (d, J=2.0 Hz, 1H), 8.59 (s, 2H), 8.09 (d, J=2.0 Hz, 1H), 7.03 (s, 1H), 7.01 (s, 1H), 6.00 (q, J=6.4 Hz, 1H), 4.85-4.68 (m, 1H), 4.23-4.20 (m, 2H), 4.11-4.09 (m, 2H), 3.86 (s, 3H), 3.57-3.49 (m, 2H), 2.80-2.70 (m, 2H), 1.76 (d, J=6.8 Hz, 3H), 1.49 (s, 3H).

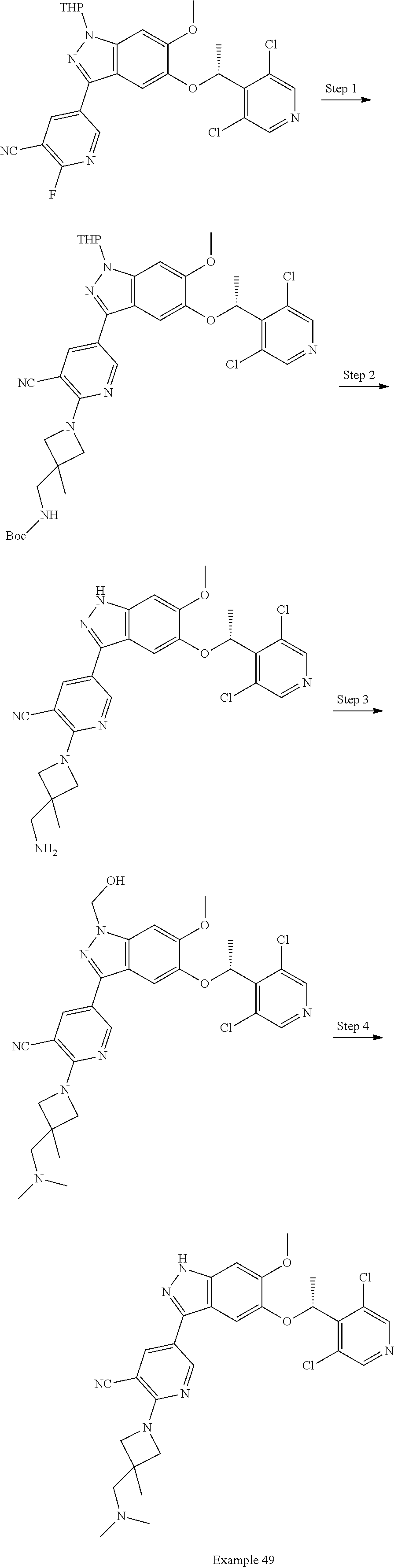

### Example 49. (R)-5-(5-(1-(3,5-dichloropyridin-4-yl)ethoxy)-6-methoxy-1H-indazol-3-yl)-2-(3-((dimethylamino)methyl)-3-methylazetidin-1-yl)nicotinonitrile

Step 1. 5-[5-[(1R)-1-(3,5-dichloro-4-pyridyl)ethoxy]-6-methoxy-1-tetrahydropyran-2-yl-indazol-3-yl]-2-[3-methyl-3-(methylaminomethyl)azetidin-1-yl]pyridine-3-carbonitrile. To a solution of 5-[5-[(1R)-1-(3,5-dichloro-4-pyridyl)ethoxy]-6-methoxy-1-tetrahydropyran-2-yl-indazol-3-yl]-2-fluoro-pyridine-3-carbonitrile (600 mg, 1.11 mmol, 1.0 eq) and tert-butyl N-[(3-methylazetidin-3-yl)methyl]carbamate HCl (525 mg, 2.22 mmol, 2.0 eq) in DMSO (15 mL) was added DIEA (2.2 g, 16.7 mmol, 15.0 eq) at room temperature. The mixture was stirred for 3 h at 60° C. After the reaction was completed, the mixture was poured into water (50 mL), and extracted with EtOAc (50 mL×3). The combined organic layer was washed with brine, dried over Na₂SO₄, and concentrated. The crude product was purified by silica gel chromatography eluted with DCM/MeOH (20/1) to give a white solid (500 mg, 63% yield). LCMS m/z=722.4 (M+H).

Step 2. 2-[3-(aminomethyl)-3-methyl-azetidin-1-yl]-5-[5-[(1R)-1-(3,5-dichloro-4-pyridyl)ethoxy]-6-methoxy-1H-indazol-3-yl]pyridine-3-carbonitrile. To a solution of 5-[5-[(1R)-1-(3,5-dichloro-4-pyridyl)ethoxy]-6-methoxy-1-tetrahydropyran-2-yl-indazol-3-yl]-2-[3-methyl-3-(methylaminomethyl)azetidin-1-yl]pyridine-3-carbonitrile (460 mg, 0.64 mmol, 1.0 eq) in DCM (4 mL) was added TFA (2 mL) at 0° C. The solution was stirred for 4 h at rt, and was concentrated in vacuum to give a yellow solid (550 mg). LCMS m/z=538.2 (M+H).

Step 3. 5-[5-[(1R)-1-(3,5-dichloro-4-pyridyl)ethoxy]-1-(hydroxymethyl)-6-methoxy-indazol-3-yl]-2-[3-[(dimethylamino)methyl]-3-methyl-azetidin-1-yl]pyridine-3-carbonitrile. To a solution of product step 2 (200 mg, 0.37 mmol, 1.0 eq) and HCHO (221 mg, 1.85 mmol, 5.0 eq, 37% aqueous solution) in DCE (4 mL) was added NaBH₃CN (93 mg, 1.50 mmol, 4.0 eq) and AcOH (2.4 mg, 0.04 mmol, 0.1 eq). The reaction mixture was stirred for 3 h at rt, poured into a saturated aqueous of NaHCO₃ (10 mL), and extracted with EtOAc (20 mL×3). The combined organic layers were washed with brine, dried over Na₂SO₄, and concentrated to give a yellow solid (130 mg). LCMS m/z=596.1 (M+H).

Step 4. (R)-5-(5-(1-(3,5-dichloropyridin-4-yl)ethoxy)-6-methoxy-1H-indazol-3-yl)-2-(3-((dimethylamino)methyl)-3-methylazetidin-1-yl)nicotinonitrile A solution of product step 3 (100 mg, 0.17 mmol, 1.0 eq) in NH₃/MeOH (3 mL) was stirred for 4 h at rt. After the reaction completion, the reaction mixture was poured into water (10 mL), and was extracted with DCM (20 mL×3). The combined organic layers were washed with brine, dried over Na₂SO₄, and concentrated. The crude product was first purified by Prep-TLC (DCM/MeOH=12/1) and then by Prep-HPLC (Prep-C18, 5 μM Triart column, 20×150 mm, YMC-Actus; gradient elution of 50% MeCN in water to 80% MeCN in water containing 0.05% NH₃·H₂O over a 10 min period to give a white solid (19 mg, 33% yield). LCMS m/z=566.3 (M+H); 1H NMR (400 MHz, DMSO-d6) δ 12.97 (s, 1H), 8.71 (d, J=2.8 Hz, 1H), 8.59 (s, 2H), 8.06 (d, J=2.4 Hz, 1H), 7.04 (s, 1H), 7.01 (s, 1H), 6.00 (q, J=6.8 Hz, 1H), 4.07 (d, J=8.4 Hz, 2H), 3.98 (d, J=8.8 Hz, 2H), 3.86 (s, 3H), 3.28-3.32 (m, 2H), 2.18 (s, 6H), 1.76 (d, J=6.8 Hz, 3H), 1.38 (s, 3H).

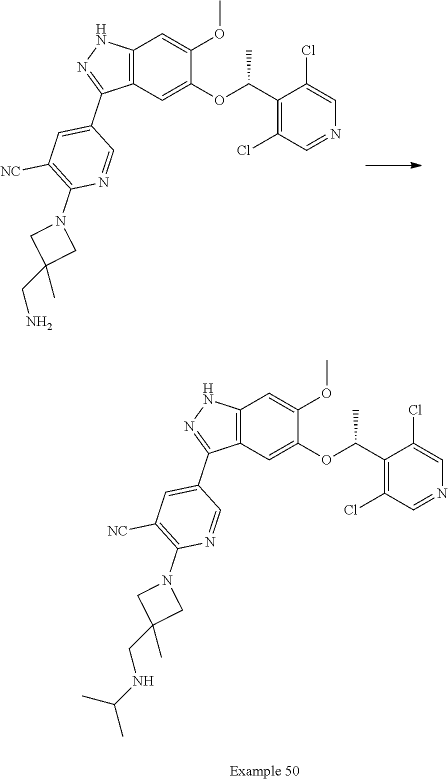 [SMILES: COc1cc2[nH]nc(-c3cnc(N4CC(C)(CN)C4)c(C#N)c3)c2cc1O[C@H](C)c1c(Cl)cncc1Cl ; COc1cc2[nH]nc(-c3cnc(N4CC(C)(CNC(C)C)C4)c(C#N)c3)c2cc1O[C@H](C)c1c(Cl)cncc1Cl]

### Example 50. (R)-5-(5-(1-(3,5-dichloropyridin-4-yl)ethoxy)-6-methoxy-1H-indazol-3-yl)-2-(3-((isopropylamino)methyl)-3-methylazetidin-1-yl)nicotinonitrile

To a solution of 2-[3-(aminomethyl)-3-methyl-azetidin-1-yl]-5-[5-[(1R)-1-(3,5-dichloro-4-pyridyl)ethoxy]-6-methoxy-1H-indazol-3-yl]pyridine-3-carbonitrile (200 mg, 0.37 mmol, 1.0 eq) and acetone (108 mg, 1.85 mmol, 5.0 eq) in DCE (4 mL) was added NaBH(AcO)₃ (318 mg, 1.50 mmol, 4.0 eq) and AcOH (2.4 mg, 0.04 mmol, 0.1 eq). The reaction mixture was stirred for 2 h at rt, poured into a saturated aqueous of NaHCO₃ (10 mL), and extracted with DCM (20 mL×3). The combined organic layers were washed with brine, dried over Na₂SO₄ and concentrated. The product was purified by Prep-TLC (DCM/MeOH=12/1) to give an off-white solid (48 mg, 22% yield). LCMS m/z=580.3 (M+H); 1H NMR (400 MHz, DMSO-d6) δ 12.99 (s, 1H), 8.71 (d, J=2.4 Hz, 1H), 8.59 (s, 2H), 8.06 (d, J=2.4 Hz, 1H), 7.03 (s, 1H) 7.01 (s, 1H), 6.01 (q, J=7.2 Hz, 1H), 4.17 (d, J=8.4 Hz, 2H), 3.92 (d, J=8.0 Hz, 2H), 3.86 (s, 3H), 3.21-3.32 (2H), 2.93-2.80 (m, 1H), 1.76 (d, J=6.8 Hz, 3H), 1.35 (s, 3H), 1.10 (s, 6H).

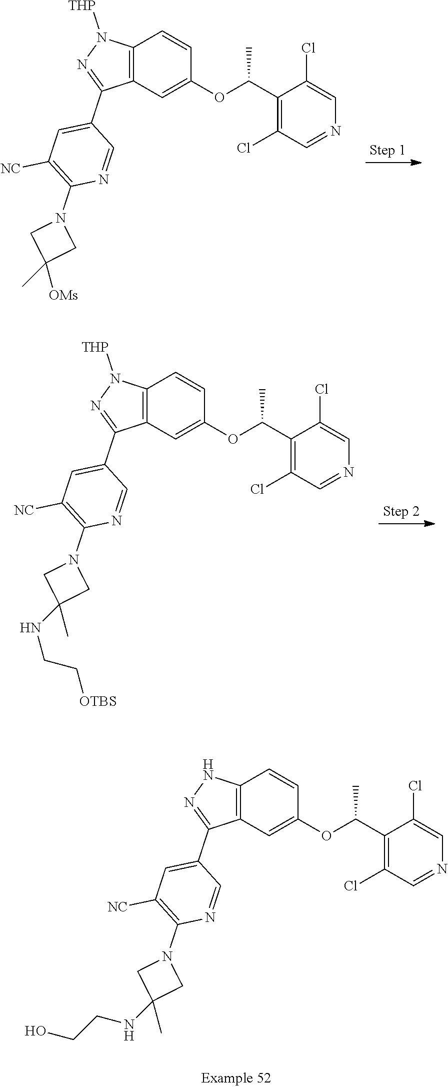

### Example 52. 5-[5-[(1R)-1-(3,5-dichloro-4-pyridyl)ethoxy]-1H-indazol-3-yl]-2-[3-(2-hydroxyethylamino)-3-methyl-azetidin-1-yl]pyridine-3-carbonitrile

Step 1. 2-(3-((2-((tert-Butyldimethylsilyl)oxy)ethyl)amino)-3-methylazetidin-1-yl)-5-(5-((R)-1-(3,5-dichloropyridin-4-yl)ethoxy)-1-(tetrahydro-2H-pyran-2-yl)-1H-indazol-3-yl)nicotinonitrile. A mixture of [1-[3-cyano-5-[5-[(1R)-1-(3,5-dichloro-4-pyridyl)ethoxy]-1-tetrahydropyran-2-yl-indazol-3-yl]-2-pyridyl]-3-methyl-azetidin-3-yl]methanesulfonate (290 mg, 0.44 mmol, 1.0 equiv), 2-[tert-butyl(dimethyl)silyl]oxyethanamine (386 mg, 2.2 mmol, 5 equiv) and sodium tert-butoxide (127 mg, 1.32 mmol, 3 equiv) in acetonitrile (5 mL) was heated at 60° C. After 18 hours, the mixture was cooled to room temperature and diluted with water (25 mL) and ethyl acetate (15 mL). The two layers were separated and the aqueous layer was extracted with ethyl acetate (2×10 mL). The combined organic layers were washed with saturated brine (25 mL), dried over magnesium sulfate, filtered and concentrated under reduced pressure. The residue was purified on a Biotage automated chromatography system (Biotage 25 g silica gel column, 60 μM), eluting with a gradient of 0 to 80% ethyl acetate in hexanes to give a light-yellow solid (100 mg, 30% yield). LCMS m/z=736 (M+H).

Step 2. 5-[5-[(1R)-1-(3,5-dichloro-4-pyridyl)ethoxy]-1H-indazol-3-yl]-2-[3-(2-hydroxyethylamino)-3-methyl-azetidin-1-yl]pyridine-3-carbonitrile. A solution of product step 1 (100 mg, 0.14 mmol, 1.0 equiv) in dichloromethane (5 mL) was treated with trifluoroacetic acid (4 mL) at room temperature. After 16 hours, the reaction mixture was concentrated under reduced pressure. The residue was treated with a mixture of saturated sodium carbonate (20 mL) and aqueous 20% sodium hydroxide (15 mL). The mixture was extracted with dichloromethane (2×15 mL). The combined organic layers were washed with saturated brine (20 mL), dried over magnesium sulfate, filtered and concentrated. The residue was purified on a Biotage automated chromatography system (RediSep Rf GOLD 100 g HP C18 column), eluting with a gradient of 0 to 100% acetonitrile in water to give an off-white solid after lyophilization (36 mg, 49% yield). LCMS m/z=538.1 (M+H); 1H NMR (400 MHz, CDCl3) δ=13.17 (br s, 1H), 8.76 (d, J=2.3 Hz, 1H), 8.58 (s, 2H), 8.13 (d, J=2.3 Hz, 1H), 7.49 (d, J=9.0 Hz, 1H), 7.16 (d, J=2.2 Hz, 1H), 7.10 (dd, J=2.3, 9.0 Hz, 1H), 6.14 (q, J=6.7 Hz, 1H), 4.53 (br s, 1H), 4.15-4.11 (m, 2H), 4.07-4.04 (m, 2H), 3.47 (br t, J=5.6 Hz, 2H), 2.62 (br d, J=5.1 Hz, 2H), 2.34-2.29 (m, 1H), 1.76 (d, J=6.7 Hz, 3H), 1.43 (s, 3H).

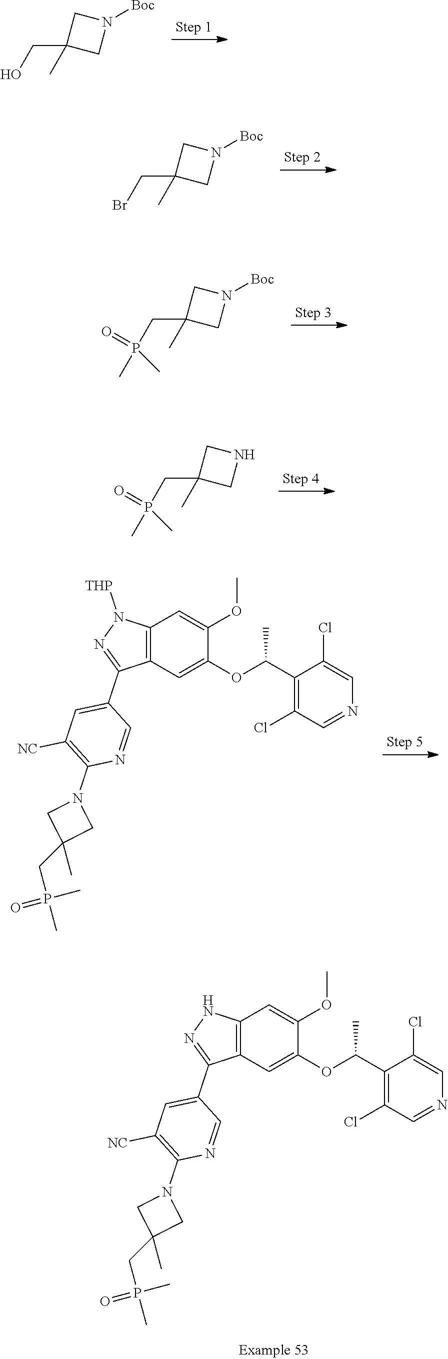

### Example 53. (R)-5-(5-(1-(3,5-Dichloropyridin-4-yl)ethoxy)-6-methoxy-1H-indazol-3-yl)-2-(3-((dimethylphosphoryl)methyl)-3-methylazetidin-1-yl)nicotinonitrile

Step 1. tert-Butyl 3-(bromomethyl)-3-methyl-azetidine-1-carboxylate. To a solution of tert-butyl 3-(hydroxymethyl)-3-methyl-azetidine-1-carboxylate (200 mg, 1.0 mmol, 1.0 eq) and PPh₃ (521 mg, 1.99 mmol, 2.0 eq) in DCM (5.0 mL) was added CBr₄ (653 mg, 1.99 mmol, 2.0 eq) in portions at 0° C. The reaction mixture was stirred at room temperature for 5 h. After the reaction was completed, the mixture was concentrated in vacuum. The crude product was purified by silica gel flash column chromatography (Petroleum Ether/EtOAc=5/1) to give a brown solid (182 mg, 69% yield). LCMS m/z=264.3 (M+H).

Step 2. tert-butyl 3-(Dimethylphosphorylmethyl)-3-methyl-azetidine-1-carboxylate. To a solution of methylphosphonoylmethane (144 mg, 1.84 mmol, 3.0 eq) in THE (2 mL) was added NaHMDS (0.92 mL, 1.84 mmol, 2 M in THF, 3.0 eq) dropwise at −70° C. under N₂ protection. The reaction mixture was stirred at room temperature for 1 h. A solution of tert-butyl 3-(bromomethyl)-3-methyl-azetidine-1-carboxylate (162 mg, 0.62 mmol, 1.0 eq) in THE (2 mL) was added dropwise at 0° C. under N₂ protection and stirred for 4 h. After the reaction was completed, the reaction was quenched with saturated NH₄Cl aqueous solution (10 mL) at 0° C. and extracted with EtOAc (20 mL×3). The combined organic layers were washed with brine (30 mL×2), dried over Na₂SO₄ and concentrated to give an off-white oil (150 mg, 93% yield). LCMS m/z=262.3 (M+H).

Step 3. 3-(Dimethylphosphorylmethyl)-3-methyl-azetidine. To a solution of tert-butyl 3-(dimethylphosphorylmethyl)-3-methyl-azetidine-1-carboxylate (150 mg, 0.57 mmol, 1.0 eq) in DCM (5.0 mL) was added TFA (1.0 mL) and stirred at room temperature for 3 h. The mixture was concentrated to give a yellow oil as the TFA salt. LCMS m/z=162.3 (M+H).

Step 4. 5-[5-[(1R)-1-(3,5-Dichloro-4-pyridyl)ethoxy]-6-methoxy-1-tetrahydropyran-2-yl-indazol-3-yl]-2-[3-(dimethylphosphorylmethyl)-3-methyl-azetidin-1-yl]pyridine-3-carbonitrile. To a solution of 5-[5-[(1R)-1-(3,5-dichloro-4-pyridyl)ethoxy]-6-methoxy-1-tetrahydropyran-2-yl-indazol-3-yl]-2-fluoro-pyridine-3-carbonitrile (150 mg, 0.28 mmol, 1.0 eq) and 3-(Dimethylphosphorylmethyl)-3-methyl-azetidine TFA salt (190 mg, crude) in DMSO (5 mL) was added DIEA (179 mg, 1.39 mmol, 5.0 eq). The reaction mixture was stirred for 16 h at 90° C. After the reaction was completed, the reaction was diluted with EtOAc (20 mL), washed with brine (20 mL×3) and concentrated. The crude product was purified by prep-TLC (DCM/MeOH=15/1) to give a yellow solid (105 mg, 56% yield). LCMS m/z=683.2 (M+H).

Step 5. (R)-5-(5-(1-(3,5-Dichloropyridin-4-yl)ethoxy)-6-methoxy-1H-indazol-3-yl)-2-(3-((dimethylphosphoryl)methyl)-3-methylazetidin-1-yl)nicotinonitrile. To a solution of product step 4 (95 mg, 0.14 mmol, 1.0 eq) in DCM (2 mL) was added TFA (0.4 mL) and stirred at room temperature for 3 h. After the reaction was completed, the reaction mixture was concentrated in vacuum. The residue was adjusted pH to 7-8 with saturated NaHCO₃ solution, and extracted with DCM (10 mL×3). The combined organic layers were concentrated in vacuum. The crude product was purified by Prep-HPLC (Prep-C18, 5 μM Triart column, 20×150 mm, YMC-Actus; gradient elution of 15% MeCN in water to 30% MeCN in water with 0.05% NH₃·H₂O over a 11 min period to give a white solid (49 mg, 58% yield). LCMS m/z=599.3 (M+H); 1H NMR (400 MHz, DMSO-d6) δ 12.96 (s, 1H), 8.71 (d, J=2.4 Hz, 1H), 8.59 (s, 2H), 8.06 (d, J=2.4 Hz, 1H), 7.03 (s, 1H), 7.00 (s, 1H), 5.99 (q, J=6.8 Hz, 1H), 4.27 (d, J=8.4 Hz, 2H), 4.03 (d, J=8.8 Hz, 2H), 3.85 (s, 3H), 2.23 (d, J=11.2 Hz, 2H), 1.76 (d, J=6.8 Hz, 3H), 1.56 (s, 3H), 1.46 (s, 3H), 1.43 (s, 3H).

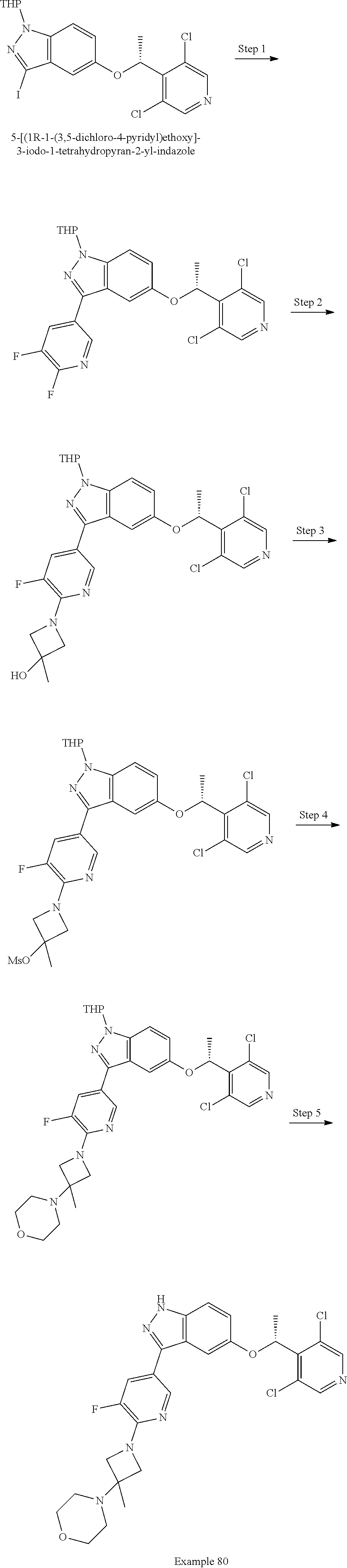

### Example 80. (R)-4-(1-(5-(5-(1-(3,5-Dichloropyridin-4-yl)ethoxy)-1H-indazol-3-yl)-3-fluoropyridin-2-yl)-3-methylazetidin-3-yl)morpholine

Step 1. 5-[(1R)-1-(3,5-dichloro-4-pyridyl)ethoxy]-3-(5,6-difluoro-3-pyridyl)-1-tetrahydropyran-2-yl-indazole. To a solution of 5-[(1R)-1-(3,5-dichloro-4-pyridyl)ethoxy]-3-iodo-1-tetrahydropyran-2-yl-indazole (4.50 g, 8.68 mmol, 1.0 eq) and 2,3-difluoro-5-(4,4,5,5-tetramethyl-1,3,2-dioxaborolan-2-yl)pyridine (3.14 g, 13.03 mmol, 1.5 eq) in dioxane (50 mL) and water (5 mL) was added K₂CO₃ (3.60 g, 26.05 mmol, 3.0 eq) and Pd(dppf)Cl₂ (708 mg, 0.87 mmol, 0.1 eq). The reaction mixture was stirred for 3 h at 90° C. under N₂ protection. After the reaction was completed, the solid was filtered out and the filtrate was concentrated in vacuum. The crude product was purified by silica gel column (Petroleum Ether/EtOAc=10/1) to give a white solid (2.3 g, 52% yield). LCMS m/z=505.2 (M+H).

Step 2. 1-[5-[5-[(1R)-1-(3,5-dichloro-4-pyridyl)ethoxy]-1-tetrahydropyran-2-yl-indazol-3-yl]-3-fluoro-2-pyridyl]-3-methyl-azetidin-3-ol. To a solution of product step 1 (2.0 g, 3.96 mmol, 1.0 eq) and 3-methylazetidin-3-ol (587 mg, 4.75 mol, 1.2 eq) in DMSO (20 mL) was added DIEA (2.56 g, 19.79 mmol, 5.0 eq). The reaction mixture was stirred for 4 h at 100° C. The reaction was cooled to room temperature, diluted with EtOAc (30 mL) and washed with brine (30 mL×3). The organic layer was dried over Na₂SO₄ and concentrated. The crude product was purified by silica gel column (DCM/MeOH=40/1) to give a white solid (1.5 g, 66% yield). LCMS m/z=572.1 (M+H).

Step 3. [1-[5-[5-[(1R)-1-(3,5-dichloro-4-pyridyl)ethoxy]-1-tetrahydropyran-2-yl-indazol-3-yl]-3-fluoro-2-pyridyl]-3-methyl-azetidin-3-yl]methanesulfonate. To a solution of product step 2 (1.5 g, 2.61 mmol, 1.0 eq) and TEA (793 mg, 7.83 mmol, 3.0 eq) in DCM (20 mL) was added MsCl (449 mg, 3.92 mmol, 1.5 eq) dropwise at 0° C. The reaction mixture was stirred for 2 h at rt. After the reaction was completed, the mixture was diluted with water (20 mL) and extracted with DCM (20 mL×2). The combined organic layers were dried over Na₂SO₄ and concentrated. The crude product was purified by silica gel column (DCM/MeOH=40/1) to give a white solid (1.4 g, 82% yield). LCMS m/z=650.2 (M+H).

Step 4. 4-[1-[5-[5-[(1R)-1-(3,5-Dichloro-4-pyridyl)ethoxy]-1-tetrahydropyran-2-yl-indazol-3-yl]-3-fluoro-2-pyridyl]-3-methyl-azetidin-3-yl]morpholine. To a solution of product step 3 (200 mg, 0.31 mmol, 1.0 eq) and morpholine (2.70 g, 3.07 mmol, 10.0 eq) in MeCN (5 mL) was added t-BuONa (148 mg, 1.54 mmol, 5.0 eq) and DMAP (75 mg, 0.62 mmol, 2.0 eq). The reaction mixture was stirred for 16 h at 80° C. The reaction was cooled to room temperature, diluted with EtOAc (50 mL) and washed with water (50 mL×3). The organic layer was dried over Na₂SO₄ and concentrated in vacuum. The crude product was purified by prep-TLC (DCM/MeOH=30/1) to give a yellow solid (70 mg, 36% yield). LCMS m/z=641.2 (M+H).

Step 5. 4-[1-[5-[5-[(1R)-1-(3,5-dichloro-4-pyridyl)ethoxy]-1H-indazol-3-yl]-3-fluoro-2-pyridyl]-3-methyl-azetidin-3-yl]morpholine. To a solution of product step 4 (70 mg, 0.11 mmol, 1.0 eq) in DCM (3 mL) was added TFA (1 mL). The reaction mixture was stirred for 3 h at rt. After the reaction was completed, the solution was concentrated. The crude product was treated with NaHCO₃ aqueous solution to adjust pH to 7-8, and extracted with DCM (20 mL×3). The combined organic layers were washed with brine, dried over Na2SO x and concentrated in vacuum. The crude product was purified by Pre-HPLC (Prep-C18, 5 μM Triart column, 20×150 mm, YMC-Actus; gradient elution of 40% MeCN in water to 50% MeCN in water containing 0.05% NH₃·H₂O over a 6 min period to give a white solid (20 mg, 34% yield). LCMS m/z=557.3 (M+H); 1H NMR (400 MHz, DMSO-d6) δ 13.12 (s, 1H), 8.57 (s, 2H), 8.36 (s, 1H), 7.71 (dd, J=13.2 Hz, 1.2 Hz, 1H), 7.47 (d, J=8.8 Hz, 1H), 7.19 (d, J=1.6 Hz, 1H), 7.10 (dd, J=8.8 Hz, 2.0 Hz, 1H), 6.14 (q, J=6.4 Hz, 1H), 3.98 (d, J=7.6 Hz, 2H), 3.81 (dd, J=8.0 Hz, 1.6 Hz, 2H), 3.61-3.59 (m, 4H), 2.43-2.41 (m, 4H), 1.76 (d, J=6.8 Hz, 3H), 1.35 (s, 3H).

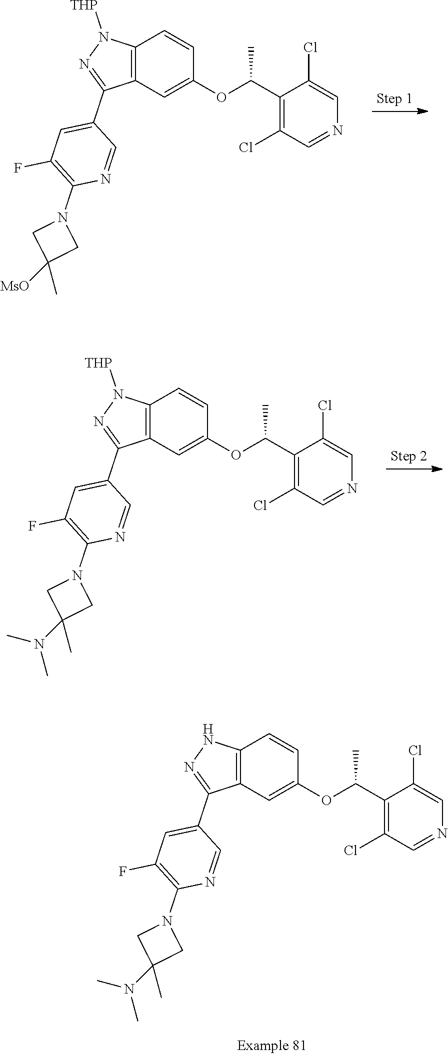

### Example 81. (R)-1-(5-(5-(1-(3,5-dichloropyridin-4-yl)ethoxy)-1H-indazol-3-yl)-3-fluoropyridin-2-yl)-N,N,3-trimethylazetidin-3-amine

Step 1. 1-[5-[5-[(1R)-1-(3,5-dichloro-4-pyridyl)ethoxy]-1-tetrahydropyran-2-yl-indazol-3-yl]-3-fluoro-2-pyridyl]-N,N,3-trimethyl-azetidin-3-amine. To a solution of [1-[5-[5-[(1R)-1-(3,5-dichloro-4-pyridyl)ethoxy]-1-tetrahydropyran-2-yl-indazol-3-yl]-3-fluoro-2-pyridyl]-3-methyl-azetidin-3-yl]methanesulfonate (300 mg, 0.46 mmol, 1.0 eq) and dimethylamine hydrochloride (752 mg, 9.22 mmol, 20.0 eq) in MeCN (5 mL) was added t-BuONa (1.11 g, 11.53 mmol, 25.0 eq). The reaction mixture was stirred for 16 h at 80° C. After the reaction was completed, the mixture was cooled to rt, diluted with EtOAc (30 mL) and washed with water (30 mL×3). The organic layer was dried over Na₂SO₄ and concentrated. The crude product was purified by prep-TLC (DCM/MeOH=30/1) to give a yellow solid (80 mg, 29% yield). LCMS m/z=599.3 (M+H).

Step 2. (R)-1-(5-(5-(1-(3,5-dichloropyridin-4-yl)ethoxy)-1H-indazol-3-yl)-3-fluoropyridin-2-yl)-N,N,3-trimethylazetidin-3-amine. To a solution of product step 1 (80 mg, 0.11 mmol, 1.0 eq) in DCM (3 mL) was added TFA (1 mL). The reaction mixture was stirred for 3 h at rt. After the reaction was completed, the reaction mixture was concentrated. The crude product was treated with NaHCO₃ (aqueous) to adjust pH to 7-8, which was extracted with DCM (20 mL×3). The combined organic layers were washed with brine, dried over Na₂SO₄ and concentrated in vacuum. The crude product was purified by Pre-HPLC (Prep-C18, 5 μM Triart column, 20×150 mm, YMC-Actus; gradient elution of 40% MeCN in water to 55% MeCN in water containing 0.05% NH₃·H₂O over a 6 min period, to give a white solid (30 mg, 34% yield). LCMS m/z=440.3 (M+H); 1H NMR (400 MHz, DMSO-d6) δ 13.12 (s, 1H), 8.57 (s, 2H), 8.36 (t, J=1.6 Hz, 1H), 7.70 (dd, J=13.2 Hz, 2.0 Hz, 1H), 7.47 (d, J=8.8 Hz, 1H), 7.20 (d, J=2.0 Hz, 1H), 7.10 (dd, J=9.2 Hz, 2.4 Hz, 1H), 6.14 (q, J=6.8 Hz, 1H), 3.93 (d, J=8.0 Hz, 2H), 3.80 (dd, J=8.4 Hz, 2.0 Hz, 2H), 2.11 (s, 6H), 1.76 (d, J=6.8 Hz, 3H), 1.32 (s, 3H).

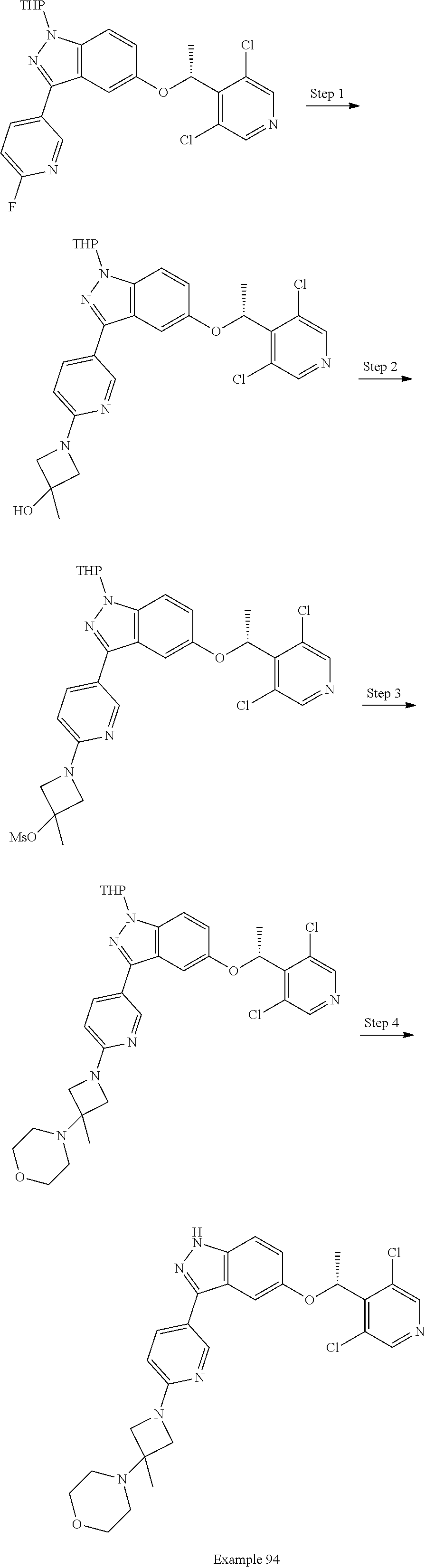

### Example 94. 4-[1-[5-[5-[(1R)-1-(3,5-dichloro-4-pyridyl)ethoxy]-1H-indazol-3-yl]-2-pyridyl]-3-methyl-azetidin-3-yl]morpholine

Step 1. 1-(5-(5-((R)-1-(3,5-Dichloropyridin-4-yl)ethoxy)-1-(tetrahydro-2H-pyran-2-yl)-1H-indazol-3-yl)pyridin-2-yl)-3-methylazetidin-3-ol. A solution of 5-[(1R)-1-(3,5-dichloro-4-pyridyl)ethoxy]-3-(6-fluoro-3-pyridyl)-1-tetrahydropyran-2-yl-indazole (793 mg, 1.63 mmol, 1.0 equiv) in N-methylpyrrolidone (2 mL) was treated sequentially with potassium carbonate (670.0 mg, 4.89 mmol, 3.0 equiv) and 3-methylazetidin-3-ol (300 mg, 2.45 mmol, 1.5 equiv). The resulting suspension was stirred at 120° C. for 16 hours. The reaction mixture was cooled to room temperature and diluted with water (5 mL). The resulting slurry was stirred at 5° C. for 1 hour. The resulting solids were filtered washed with water (10 mL) and dried under vacuum at 50° C. for 4 hours to give) an off-white solid (858 mg, 95% yield). This material was used directly in the next step. LCMS m/z=554.1 (M+H).

Step 2. 1-(5-(5-((R)-1-(3,5-Dichloropyridin-4-yl)ethoxy)-1-(tetrahydro-2H-pyran-2-yl)-1H-indazol-3-yl)pyridin-2-yl)-3-methylazetidin-3-yl methanesulfonate. N,N-Diisopropylethylamine (0.8 mL, 4.62 mmol, 3.0 equiv) and methanesulfonic anhydride (540 mg, 3.09 mmol, 2.0 equiv) were sequentially added to a solution of product step 1 (858 mg, 1.54 mmol, 1.0 equiv) in dichloromethane (10 mL) at 0° C. The resulting solution was stirred at room temperature for 2 days. The crude reaction was diluted with saturated sodium bicarbonate (10 mL). The layers were separated and the aqueous layer was extracted with dichloromethane (10 mL). The combined organic layers were dried over sodium sulfate, filtered and concentrated under reduced pressure. The residue was purified on a Buchi automated chromatography system (Sorbtech, 40 g column), eluting with a gradient of 0 to 50% ethyl acetate in heptanes to give a white solid (500 mg, 49% yield). LCMS m/z=631.1 (M+H).

Step 3. 4-(1-(5-(5-((R)-1-(3,5-Dichloropyridin-4-yl)ethoxy)-1-(tetrahydro-2H-pyran-2-yl)-1H-indazol-3-yl)pyridin-2-yl)-3-methylazetidin-3-yl)morpholine. In a sealed 50 mL tube, a solution of product step 2 (500 mg, 0.79 mmol, 1.0 equiv) in acetonitrile was treated sequentially with morpholine (0.28 mL, 3.16 mmol, 4.0 equiv) and sodium tert-butoxide (150 mg, 1.58 mmol, 2.0 equiv). The resulting suspension was heated at 60° C. for 16 hours. Additional morpholine (0.69 mL, 7.9 equiv, 10.0 equiv) and sodium tert-butoxide (380 mg, 3.95 equiv, 5.0 equiv) were added and the reaction continued for 2 days. After cooling to room temperature, the volatiles were removed under reduced pressure. The residue was dissolved in ethyl acetate (30 mL) and washed with saturated sodium bicarbonate (15 mL). The aqueous layer was extracted with ethyl acetate (15 mL). The combined organic layers were dried over sodium sulfate, filtered and concentrated under reduced pressure. The residue was purified on a Buchi automated chromatography system (Sorbtech 40 g column), eluting with a gradient of 0 to 10% methanol in dichloromethane to give a light brown solid (118 mg, 24% yield). LCMS m/z=622.2 (M+H).

Step 4. (R)-4-(1-(5-(5-(1-(3,5-Dichloropyridin-4-yl)ethoxy)-1H-indazol-3-yl)pyridin-2-yl)-3-methylazetidin-3-yl)morpholine. The product step 3 (118 mg, 0.19 mmol, 1.0 equiv) was treated with a 1 to 1 mixture of dichloromethane (1.2 mL) and trifluoroacetic acid (1.2 mL, 15.7 mmol, 82.5 equiv) at room temperature for 6 hours. The volatiles were removed under reduced pressure. The residue was diluted with dichloromethane (5 mL) then treated with water (2 mL) and sodium bicarbonate (500 mg). The mixture was concentrated under reduced pressure, absorbed onto Celite (10.0 g) and purified on a Buchi automated chromatography system (RediSep C_{18 100} g column), eluting with a gradient of 0 to 80% acetonitrile in water. Product containing fractions were pooled together and lyophilized to give a white solid (36.0 mg, 36% yield). LCMS m/z=539.2 (M+H); 1H NMR (400 MHz, DMSO-d6) δ=12.98 (s, 1H), 8.59 (s, 2H), 8.51 (d, J=1.8 Hz, 1H), 7.85 (dd, J=2.3, 8.6 Hz, 1H), 7.45 (d, J=9.0 Hz, 1H), 7.16 (s, 1H), 7.09 (dd, J=2.3, 8.9 Hz, 1H), 6.52 (d, J=8.3 Hz, 1H), 6.11 (q, J=6.7 Hz, 1H), 3.82 (d, J=7.9 Hz, 2H), 3.69 (d, J=7.8 Hz, 2H), 3.65-3.56 (m, 4H), 2.43 (br s, 4H), 1.76 (d, J=6.6 Hz, 3H), 1.34 (s, 3H)

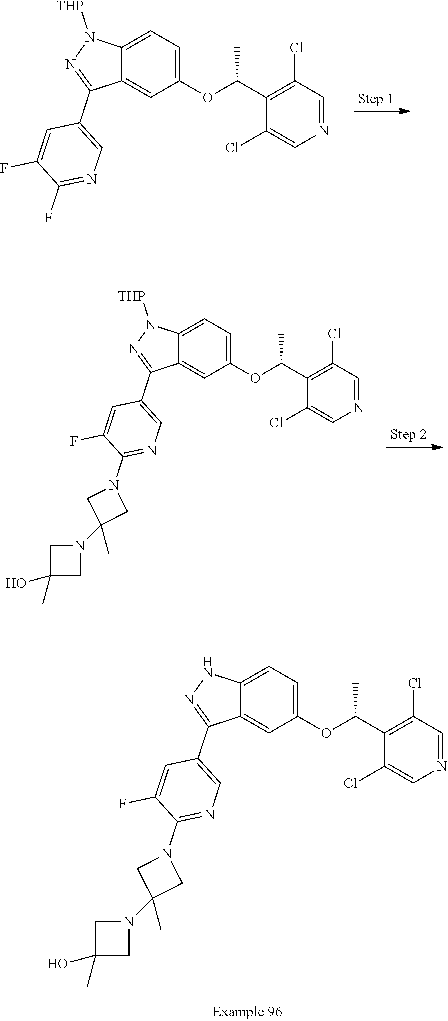

### Example 96. (R)-1′-(5-(5-(1-(3,5-dichloropyridin-4-yl)ethoxy)-1H-indazol-3-yl)-3-fluoropyridin-2-yl)-3,3′-dimethyl-[1,3′-biazetidin]-3-ol

Step 4. 1-[1-[5-[5-[(1R)-1-(3,5-dichloro-4-pyridyl)ethoxy]-1-tetrahydropyran-2-yl-indazol-3-yl]-3-fluoro-2-pyridyl]-3-methyl-azetidin-3-yl]-3-methyl-azetidin-3-ol. To a solution of 5-[(1R)-1-(3,5-dichloro-4-pyridyl)ethoxy]-3-(5,6-difluoro-3-pyridyl)-1-tetrahydropyran-2-yl-indazole (200 mg, 0.39 mmol, 1.0 eq) and 3-methyl-1-(3-methylazetidin-3-yl)azetidin-3-ol (123.7 mg, 0.79 mmol, 2.0 eq) in DMSO (5 mL) was added DIEA (306 mg, 2.37 mmol, 6.0 eq). The reaction mixture was stirred for 4 h at 100° C. After the reaction was completed, the mixture was diluted with EtOAc (20 mL) and washed with brine (20 mL×3). The organic layer was dried over Na₂SO₄ and concentrated. The crude product was purified by prep-TLC (DCM/MeOH=20/1) to give a white solid (200 mg, 79% yield). LCMS m/z=641.2 (M+H).

Step 2. (R)-1′-(5-(5-(1-(3,5-dichloropyridin-4-yl)ethoxy)-1H-indazol-3-yl)-3-fluoropyridin-2-yl)-3,3′-dimethyl-[1,3′-biazetidin]-3-ol. To a solution of product step 1 (150 mg, 0.23 mmol, 1.0 eq) in DCM (3 mL) was added TFA (1 mL), which was stirred at rt for 3 h. The reaction mixture was concentrated and the crude product was dissolved in DCM/MeOH (10 mL, V:V=5/1), and K₂CO₃ (excess) was added and stirred for 20 minutes. DCM (20 mL) was added and the solid was filtered and the filtrate was concentrated. The residue was purified by Prep-TLC (DCM:MeOH=10:1) to give a white solid (52 mg, 40% yield). LCMS m/z=557.2 (M+H); 1H NMR (400 MHz, DMSO-d6) δ 13.12 (s, 1H), 8.57 (s, 2H), 8.37 (s, 1H), 7.72 (dd, J=13.2 Hz, 2.0 Hz, 1H), 7.48 (d, J=8.8 Hz, 1H), 7.20 (d, J=1.2 Hz, 1H), 7.11 (dd, J=8.8 Hz, 2.0 Hz, 1H), 6.15 (q, J=6.4 Hz, 1H), 5.22 (s, 1H), 4.21 (d, J=8.4 Hz, 2H), 3.79 (d, J=7.6 Hz, 2H), 3.13-3.10 (m, 4H), 1.75 (d, J=6.4 Hz, 3H), 1.38 (s, 3H), 1.30 (s, 3H).

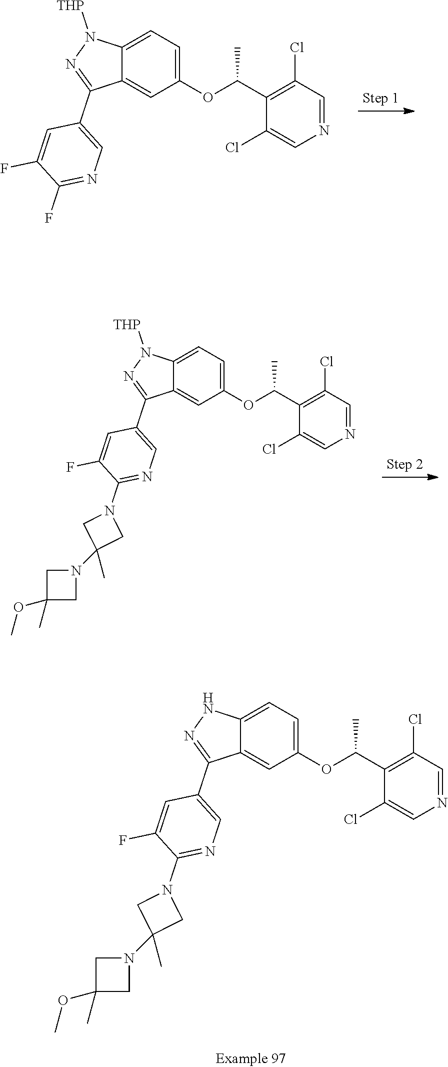

### Example 97. (R)-5-(1-(3,5-dichloropyridin-4-yl)ethoxy)-3-(5-fluoro-6-(3-methoxy-3,3′-dimethyl-[1,3′-biazetidin]-1′-yl)pyridin-3-yl)-1H-indazole

Step 1. 5-[(1R)-1-(3,5-dichloro-4-pyridyl)ethoxy]-3-[5-fluoro-6-[3-(3-methoxy-3-methyl-azetidin-1-yl)-3-methyl-azetidin-1-yl]-3-pyridyl]-1-tetrahydropyran-2-yl-indazole. To a solution of 5-[(1R)-1-(3,5-dichloro-4-pyridyl)ethoxy]-3-(5,6-difluoro-3-pyridyl)-1-tetrahydropyran-2-yl-indazole (200 mg, 0.39 mmol, 1.0 eq) and 3-methoxy-3-methyl-1-(3-methylazetidin-3-yl)azetidine (123.7 mg, 0.79 mol, 2.0 eq) in DMSO (5 mL) was added DIEA (306 mg, 2.37 mmol, 6.0 eq). The reaction mixture was stirred for 4 h at 100° C. After the reaction was completed, the mixture was diluted with EtOAc (20 mL) and washed with brine (20 mL×3). The organic layer was dried over Na₂SO₄ and concentrated. The crude product was purified by prep-TLC (DCM/MeOH=20/1) to give a white solid (200 mg, 79% yield). LCMS m/z=655.2 (M+H).

Step 2. (R)-5-(1-(3,5-dichloropyridin-4-yl)ethoxy)-3-(5-fluoro-6-(3-methoxy-3,3′-dimethyl-[1,3′-biazetidin]-1′-yl)pyridin-3-yl)-1H-indazole. To a solution of Example 850c (150 mg, 0.23 mmol, 1.0 eq) in DCM (3 mL) was added TFA (1 mL), which was stirred at r.t. for 3 h. The reaction mixture was concentrated. The crude product was treated with DCM/MeOH (10 mL, V:V=5/1), K₂CO₃ (excess) was added to the solution and stirred for 20 minutes at rt, then DCM (20 mL) was added. The solid was filtered out and the filtrate was concentrated, the residue was purified by Prep-TLC (DCM:MeOH=10:1) to give a white solid (57 mg, 44% yield). LCMS m/z=571.3 (M+H); 1H NMR (400 MHz, DMSO-d6) δ 13.12 (s, 1H), 8.56 (s, 2H), 8.37 (s, 1H), 7.70 (dd, J=13.2 Hz, 1.2 Hz, 1H), 7.47 (d, J=9.2 Hz, 1H), 7.19 (s, 1H), 7.09 (dd, J=9.2 Hz, 2.4 Hz, 1H), 6.13 (q, J=6.4 Hz, 1H), 4.12 (d, J=8.4 Hz, 2H), 3.80 (d, J=8.4 Hz, 2H), 3.19-3.17 (m, 2H), 3.12-3.08 (m, 5H), 1.75 (d, J=6.4 Hz, 3H), 1.40 (s, 3H), 1.31 (s, 3H).

TABLE
Examples synthesized using procedures described previously

| | | |
|---|---|---|
| Example 1 (R)-1-(5-(5-(1-(3,5- dichloropyridin-4- yl)ethoxy)-6-methoxy- 1H-indazol-3-y1)-3- (methylsulfonyl)pyridin- 2-yl)-N,N,3- trimethylazetidin-3- amine | 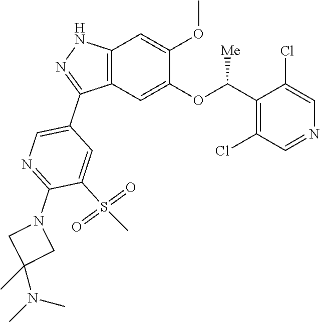 [SMILES: COc1cc2[nH]nc(-c3cnc(N4CC(C)(N(C)C)C4)c(S(C)(=O)=O)c3)c2cc1O[C@H](C)c1c(Cl)cncc1Cl] | LCMS m/z = 605.3 (M + H); 1H NMR (400 MHz, DMSO-d6) δ 13.03 (brs, 1H), 8.74 (d, J = 2.0 Hz, 1H), 8.57 (s, 2H), 8.38 (d, J = 2.0 Hz, 1H), 7.15 (s, 1H), 7.03 (s, 1H), 5.98 (q, J = 6.4 Hz, 1H), 4.12 (d, J = 8.8 Hz, 2H), 3.97 (d, J = 9.2 Hz, 2H), 3.87 (s, 3H), 3.35 (s, 3H), 2.13 (s, 6H), 1.75 (d, J = 6.8 Hz, 3H), 1.29 (s, 3H) |
| Example 2 (R)-2-(3- (cyclobutylamino)-3- methylazetidin-1-yl)-5- (5-(1-(3,5- dichloropyridin-4- yl)ethoxy)-1H-indazol- 3-yl)nicotinonitrile | 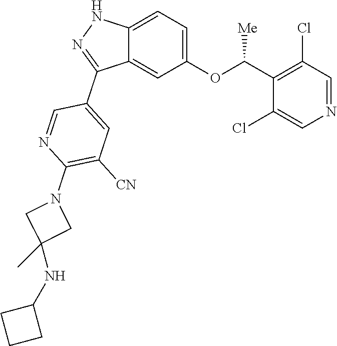 [SMILES: C[C@@H](Oc1ccc2[nH]nc(-c3cnc(N4CC(C)(NC5CCC5)C4)c(C#N)c3)c2c1)c1c(Cl)cncc1Cl] | LCMS m/z = 548.2 (M + H); 1H NMR (400 MHz, DMSO-d6) δ 13.18 (s, 1H), 8.76 (d, J = 2.0 Hz, 1H), 8.58 (s, 2H), 8.14 (d, J = 2.0 Hz, 1H), 7.49 (d, J = 9.2 Hz, 1H), 7.16 (d, J = 2.0 Hz, 1H), 7.10 (dd, J = 8.8 Hz, 2.4 Hz, 1H), 6.14 (q, J = 6.8 Hz, 1H), 4.11 (d, J = 8.4 Hz, 2H), 4.03 (d, J = 8.4 Hz, 2H), 3.31- 3.29 (m, 1H), 2.16-2.10 (m, 2H), 1.80-1.75 (m, 5H), 1.64-1.41 (m, 2H), 1.23 (s, 3H) |
| Example 3 (R)-5-(5-(1-(3,5- dichloropyridin-4- yl)ethoxy)-6-methoxy- 1H-indazol-3-yl)-2-(5- azaspiro[2.3 ]hexan-5- yl)nicotinonitrile | 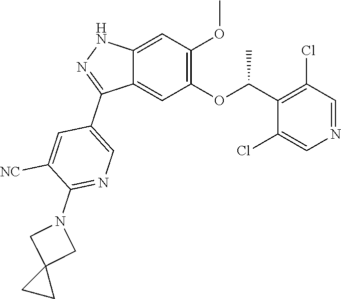 [SMILES: COc1cc2[nH]nc(-c3cnc(N4CC5(CC5)C4)c(C#N)c3)c2cc1O[C@H](C)c1c(Cl)cncc1Cl] | LCMS m/z = 521.1 (M + H); 1H NMR (400 MHz, DMSO-d6) δ 12.96 (s, 1H), 8.73 (d, J = 2.3 Hz, 1H), 8.59 (s, 2H), 8.09 (d, J = 2.3 Hz, 1H), 7.05 (s, 1H), 7.01 (s, 1H), 6.03-5.96 (m, 1H), 5.75 (s, 1H), 4.38 (s, 4H), 3.86 (s, 3H), 1.76 (d, J = 6.6 Hz, 3H), 0.73 (s, 4H) |
| Example 4 (R)-5-(5-(1-(3,5- dichloropyridin-4- yl)ethoxy)-1H-indazol- 3-yl)-2-(3-methyl-3- (oxetan-3- ylamino)azetidin-1- yl)nicotinonitrile | 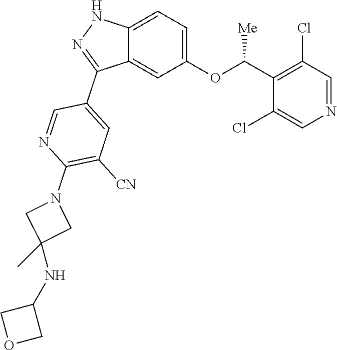 [SMILES: C[C@@H](Oc1ccc2[nH]nc(-c3cnc(N4CC(C)(NC5COC5)C4)c(C#N)c3)c2c1)c1c(Cl)cncc1Cl] | LCMS m/z = 550.4 (M + H); 1H NMR (400 MHz, DMSO-d6) δ 13.16 (s, 1H), 8.76 (d, J = 2.0 Hz, 1H), 8.57 (s, 2H), 8.14 (d, J = 2.0 Hz, 1H), 7.48 (d, J = 8.8 Hz, 1H), 7.16 (s, 1H), 7.10 (dd, J = 8.8 Hz, 1.6 Hz, 1H), 6.14 (q, J = 6.4 Hz, 1H), 4.67 (t, J = 6.8 Hz, 1H), 4.42- 4.34 (m, 2H), 4.15-3.98 (m, 5H), 1.76 (d, J = 6.4 Hz, 3H), 1.35 (s, 3H) |
| Example 5 (R)-5-(5-(1-(3,5- Dichloropyridin-4- yl)ethoxy)-6-fluoro- 1H-indazol-3-yl)-2-(5- azaspiro[2.3 ]hexan-5- yl)nicotinonitrile | 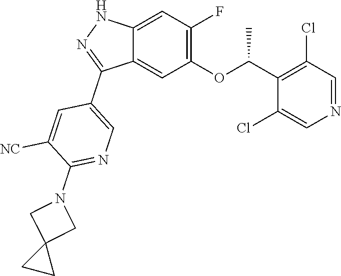 [SMILES: C[C@@H](Oc1cc2c(-c3cnc(N4CC5(CC5)C4)c(C#N)c3)n[nH]c2cc1F)c1c(Cl)cncc1Cl] | LCMS m/z = 509 (M + H); 1H NMR (400 MHz, DMSO-d6) δ = 13.23 (s, 1H), 8.75 (d, J = 2.3 Hz, 1H), 8.60 (s, 2H), 8.15 (d, J = 2.3 Hz, 1H), 7.47 (d, J = 10.9 Hz, 1H), 7.26 (d, J = 7.8 Hz, 1H), 6.18 (q, J = 6.6 Hz, 1H), 4.40 (s, 4H), 1.80 (d, J = 6.7 Hz, 3H), 0.74 (s, 4H) |
| Example 6 (R)-5-(5-(1-(3,5- dichloropyridin-4- yl)ethoxy)-1H-indazol- 3-yl)-2-(3- ((dimethylphosphoryl) methyl)-3- methylazetidin-1- yl)nicotinonitrile | 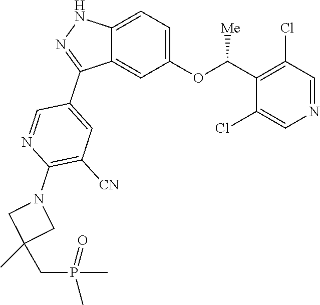 [SMILES: C[C@@H](Oc1ccc2[nH]nc(-c3cnc(N4CC(C)(CP(C)(C)=O)C4)c(C#N)c3)c2c1)c1c(Cl)cncc1Cl] | LCMS m/z = 569.4 (M + H); 1H NMR (400 MHz, DMSO-d6) δ 13.17 (brs, 1H), 8.76 (d, J = 2.4 Hz, 1H), 8.57 (s, 2H), 8.13 (d, J = 2.4 Hz, 1H), 7.48 (d, J = 8.8 Hz, 1H), 7.15 (d, J = 2.0 Hz, 1H), 7.10 (dd, J = 8.8 Hz, 2.4 Hz, 1H), 6.13 (q, J = 6.8 Hz, 1H), 4.27 (d, J = 8.4 Hz, 2H), 4.03 (d, J = 8.8 Hz, 2H), 2.23 (d, J = 10.8 Hz, 2H), 1.75 (d, J = 6.8 Hz, 3H), 1.56 (s, 3H), 1.46 (s, 3H), 1.43 (s, 3H) |
| Example 7 (R)-5-(5-(1-(3,5- dichloropyridin-4- yl)ethoxy)-1H-indazol- 3-yl)-2-(3-((1,1- dioxidothietan-3- yl)amino)-3- methylazetidin-1- yl)nicotinonitrile | 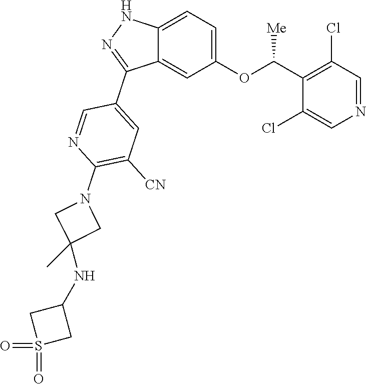 [SMILES: C[C@@H](Oc1ccc2[nH]nc(-c3cnc(N4CC(C)(NC5CS(=O)(=O)C5)C4)c(C#N)c3)c2c1)c1c(Cl)cncc1Cl] | LCMS m/z = 598.2 (M + H); 1H NMR (400 MHz, DMSO-d6) δ 13.18 (s, 1H), 8.77 (d, J = 1.2 Hz, 1H), 8.58 (d, J = 0.8 Hz, 2H), 8.15 (d, J = 1.6 Hz, 1H), 7.49 (d, J = 9.2 Hz, 1H), 7.16 (s, 1H), 7.11 (dd, J = 8.8 Hz, 1.6 Hz, 1H), 6.14 (q, J = 6.4 Hz, 1H), 4.45-4.40 (m, 2H), 4.13-4.06 (m, 4H), 3.98-3.95 (m, 2H), 3.78-3.74 (m, 1H), 1.76 (d, J = 6.8 Hz, 3H), 1.43 (s, 3H) |
| Example 8 5-[5-[(1R)-1-(3,5- dichloro-4- pyridyl)ethoxy]-6- methoxy-1H-indazol-3- yl]-2-[3- (isopropylamino)-3- methyl-azetidin-1- yl]pyridine-3- carbonitrile | 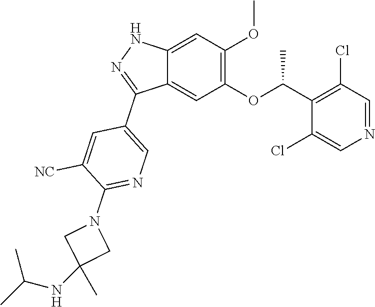 [SMILES: COc1cc2[nH]nc(-c3cnc(N4CC(C)(NC(C)C)C4)c(C#N)c3)c2cc1O[C@H](C)c1c(Cl)cncc1Cl] | LCMS m/z = 566.2 (M + H); 1H NMR (400 MHz, DMSO-d6) δ = 9.20 (br s, 2H), 8.79 (d, J = 2.2 Hz, 1H), 8.58 (s, 2H), 8.19 (d, J = 2.3 Hz, 1H), 7.06 (s, 1H), 7.02 (s, 1H), 6.03-5.97 (m, 1H), 4.49 (d, J = 9.7 Hz, 2H), 4.34 (d, J = 9.4 Hz, 2H), 3.86 (s, 3H), 3.55 (br s, 1H), 3.33 (br s, 3H), 1.79-1.75 (m, 3H), 1.26 (d, J = 6.4 Hz, 6H) |
| Example 9 5-[5-[(1R)-1-(3,5- dichloro-4- pyridyl)ethoxy]-6- methoxy-1H-indazol-3- yl]-2-[3-methyl-3- (methylamino)azetidin- 1-yl]pyridine-3- carbonitrile | 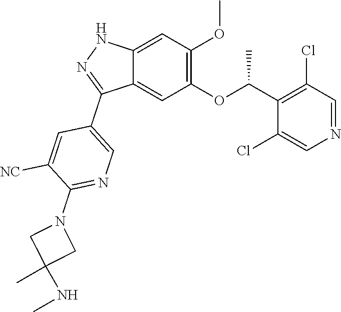 [SMILES: CNC1(C)CN(c2ncc(-c3n[nH]c4cc(OC)c(O[C@H](C)c5c(Cl)cncc5Cl)cc34)cc2C#N)C1] | LCMS m/z = 538.1 (M + H); 1H NMR (400 MHz, DMSO-d6) δ = 12.96 (s, 1H), 8.72 (d, J = 2.2 Hz, 1H), 8.59 (s, 2H), 8.07 (d, J = 2.4 Hz, 1H), 7.04 (s, 1H), 7.01 (s, 1H), 6.00 (q, J = 6.6 Hz, 1H), 4.18-4.08 (m, 2H), 4.08-3.98 (m, 2H), 3.86 (s, 3H), 2.28 (s, 3H), 1.76 (d, J = 6.8 Hz, 3H), 1.51-1.38 (m, 3H) |
| Example 10 5-[5-[(1R)-1-(3,5- dichloro-4- pyridyl)ethoxy]-6- methoxy-1H-indazol-3- yl]-2-[3- (dimethylamino)-3- methyl-azetidin-1- yl]pyridine-3- carbonitrile | 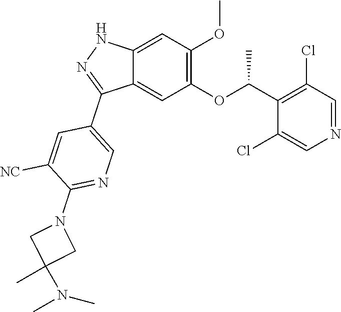 [SMILES: COc1cc2[nH]nc(-c3cnc(N4CC(C)(N(C)C)C4)c(C#N)c3)c2cc1O[C@H](C)c1c(Cl)cncc1Cl] | LCMS m/z = 552.1 (M + H); 1H NMR (400 MHz, DMSO-d6) δ = 13.04 (br s, 1H), 10.63 (br s, 1H), 8.78 (d, J = 2.2 Hz, 1H), 8.59 (s, 2H), 8.20 (d, J = 2.3 Hz, 1H), 7.06 (s, 1H), 7.03 (s, 1H), 6.03-5.97 (m, 1H), 4.51 (br d, J = 10.1 Hz, 2H), 4.26 (d, J = 10.0 Hz, 2H), 3.87 (s, 3H), 2.80 (br s, 6H), 1.77 (d, J = 6.6 Hz, 3H), 1.68 (s, 3H) |
| Example 11 (R)-5-(5-(1-(3,5- dichloropyridin-4- yl)ethoxy)-6-fluoro- 1H-indazol-3-yl)-2-(3- ((dimethylphosphoryl) methyl)-3- methylazetidin-1- yl)nicotinonitrile | 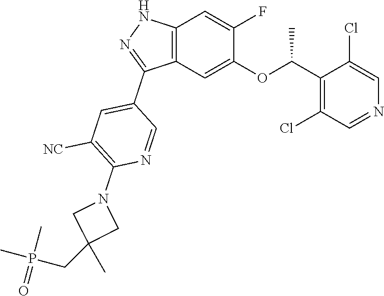 [SMILES: C[C@@H](Oc1cc2c(-c3cnc(N4CC(C)(CP(C)(C)=O)C4)c(C#N)c3)n[nH]c2cc1F)c1c(Cl)cncc1Cl] | LCMS m/z = 587.2 (M + H); 1H NMR (400 MHz, DMSO-d6) δ 13.23 (s, 1H), 8.74 (d, J = 2.0 Hz, 1H), 8.60 (s, 2H), 8.12 (d, J = 2.0 Hz, 1H), 7.46 (d, J = 10.8 Hz, 1H), 7.24 (d, J = 7.6 Hz, 1H), 6.18 (q, J = 6.8 Hz, 1H), 4.28 (d, J = 8.8 Hz, 2H), 4.04 (d, J = 8.8 Hz, 2H), 2.24 (d, J = 11.2 Hz, 2H), 1.80 (d, J = 6.4 Hz, 3H), 1.56 (s, 3H), 1.47 (s, 3H), 1.43 (s, 3H) |
| Example 12 (R)-2-(3- (cyclobutylamino)-3- methylazetidin-1-yl)-5- (5-(1-(3,5- dichloropyridin-4- yl)ethoxy)-6-methoxy- 1H-indazol-3- yl)nicotinonitrile | 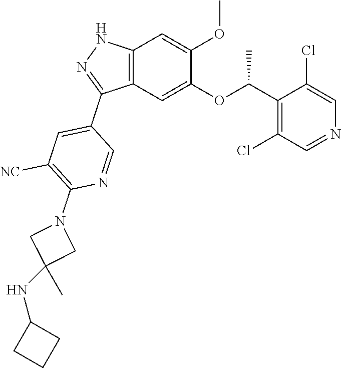 [SMILES: COc1cc2[nH]nc(-c3cnc(N4CC(C)(NC5CCC5)C4)c(C#N)c3)c2cc1O[C@H](C)c1c(Cl)cncc1Cl] | LCMS m/z = 578.3 (M + H); 1H NMR (400 MHz, DMSO-d6) δ 12.96 (s, 1H), 8.71 (d, J = 2.4 Hz, 1H), 8.59 (s, 2H), 8.06 (d, J = 2.0 Hz, 1H), 7.04 (s, 1H), 7.00 (s, 1H), 5.99 (q, J = 6.4 Hz, 1H), 4.08 (d, J = 8.4 Hz, 2H), 4.01 (d, J = 8.4 Hz, 2H), 3.86 (s, 3H), 3.23-3.29 (m, 1H), 2.15-2.09 (m, 2H), 1.77-1.71 (m, 5H), 1.61-1.54 (m, 2H), 1.40 (s, 3H). |
| Example 13 5-[5-[(1R)-1-(3,5- dichloro-2-methyl-4- pyridyl)ethoxy]-1H- indazol-3-yl]-2-(3- isopropoxy-3-methyl- azetidin-1-yl)pyridine- 3-carbonitrile | 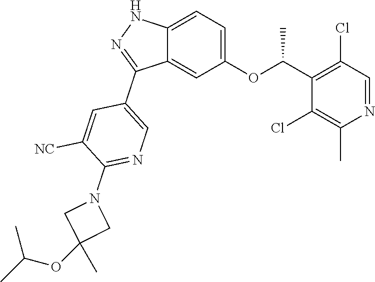 [SMILES: Cc1ncc(Cl)c([C@@H](C)Oc2ccc3[nH]nc(-c4cnc(N5CC(C)(OC(C)C)C5)c(C#N)c4)c3c2)c1Cl] | LCMS m/z = 551.4 (M + H); 1H NMR (400 MHz, DMSO-d6) δ = 13.17 (s, 1H), 8.70 (d, J = 2.2 Hz, 1H), 8.43 (s, 1H), 8.18 (d, J = 2.2 Hz, 1H), 7.49 (d, J = 9.8 Hz, 1H), 7.10 (s, 1H), 7.10 (d, J = 6.2 Hz, 2H), 6.13 (q, J = 6.6 Hz, 1H), 3.87 (quin, J = 6.1 Hz, 1H), 3.31 (s, 3H), 2.56 (s, 3H), 1.76 (d, J = 6.6 Hz, 3H), 1.57 (s, 3H), 1.14 (d, J = 6.1 Hz, 6H) |
| Example 14 (R)-2-(3-((cyclopropyl- methyl)-amino)-3- methylazetidin-1-yl)-5- (5-(1-(3,5- dichloropyridin-4- yl)ethoxy)-6-methoxy- 1H-indazol-3- yl)nicotinonitrile | 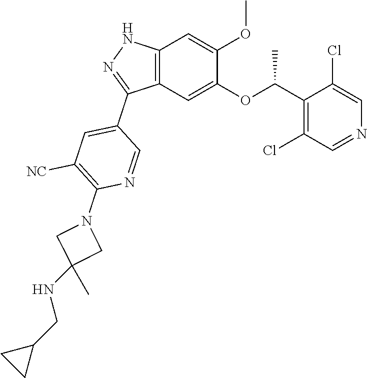 [SMILES: COc1cc2[nH]nc(-c3cnc(N4CC(C)(NCC5CC5)C4)c(C#N)c3)c2cc1O[C@H](C)c1c(Cl)cncc1Cl] | LCMS m/z = 578.3 (M + H); 1H NMR (400 MHz, DMSO-d6) δ 12.96 (brs, 1H), 8.71 (d, J = 2.0 Hz, 1H), 8.59 (s, 2H), 8.06 (d, J = 2 Hz, 1H), 7.03 (s, 1H), 7.00 (s, 1H), 6.00 (q, J = 6.4 Hz, 1H), 4.14 (d, J = 8.4 Hz, 2H), 4.02 (d, J = 8.4 Hz, 2H), 3.86 (s, 3H), 2.42 (d, J = 6.4 Hz, 2H), 1.76 (d, J = 6.4 Hz, 3H), 1.43 (s, 3H), 0.93-0.82 (m, 1H), 0.44- 0.38 (m, 2H), 0.17-0.14 (m, 2H) |
| Example 15 (R)-5-(5-(1-(3,5- dichloropyridin-4- yl)ethoxy)-6-methoxy- 1H-indazol-3-yl)-2-(3- methyl-3-(pyrrolidin-1- yl)azetidin-1- yl)nicotinonitrile | 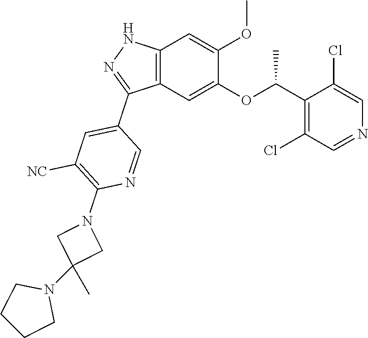 [SMILES: COc1cc2[nH]nc(-c3cnc(N4CC(C)(N5CCCC5)C4)c(C#N)c3)c2cc1O[C@H](C)c1c(Cl)cncc1Cl] | LCMS m/z = 578.3 (M + H); 1H NMR (400 MHz, DMSO-d6) δ 12.97 (brs, 1H), 8.71 (d, J = 2.0 Hz, 1H), 8.59 (s, 2H), 8.07 (d, J = 2.4 Hz, 1H), 7.04 (s, 1H), 7.00 (s, 1H), 6.00 (q, J = 6.4 Hz, 1H), 4.31-4.29 (m, 2H), 4.00-3.98 (m, 2H), 3.86 (s, 3H), 2.66-2.58 (m, 4H), 1.76 (d, J = 6.4 Hz, 3H), 1.74-1.68 (m, 4H), 1.45 (s, 3H) |
| Example 16 (R)-5-(5-(1-(3,5- dichloropyridin-4- yl)ethoxy)-6-methoxy- 1H-indazol-3-yl)-2-(3- methyl-3- morpholinoazetidin-1- yl)nicotinonitrile | 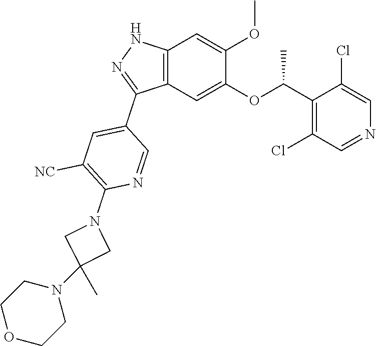 [SMILES: COc1cc2[nH]nc(-c3cnc(N4CC(C)(N5CCOCC5)C4)c(C#N)c3)c2cc1O[C@H](C)c1c(Cl)cncc1Cl] | LCMS m/z = 594.3 (M + H); 1H NMR (400 MHz, DMSO-d6) δ 12.98 (s, 1H), 8.72 (d, J = 2.0 Hz, 1H), 8.59 (s, 2H), 8.08 (d, J = 2.4 Hz, 1H), 7.03 (s, 1H), 7.00 (s, 1H), 6.00 (q, J = 6.4 Hz, 1H), 4.12-4.10 (m, 2H), 3.98-3.96 (m, 2 H), 3.86 (s, 3H), 3.62-3.60 (m, 4H), 2.44- 2.42 (m, 4H), 1.76 (d, J = 6.8 Hz, 3H), 1.34 (s, 3H). |
| Example 17 (R)-5-(5-(1-(3,5- dichloropyridin-4- yl)ethoxy)-1H-indazol- 3-yl)-2-(3-methyl-3- (pyrrolidin-1- yl)azetidin-1- yl)nicotinonitrile | 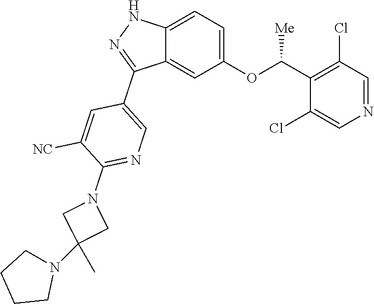 [SMILES: C[C@@H](Oc1ccc2[nH]nc(-c3cnc(N4CC(C)(N5CCCC5)C4)c(C#N)c3)c2c1)c1c(Cl)cncc1Cl] | LCMS m/z = 548.2 (M + H); 1H NMR (400 MHZ, DMSO-d6) δ 13.17 (s, 1H), 8.76 (d, J = 2.4 Hz, 1H), 8.57 (s, 2H), 8.14 (d, J = 2.4 Hz, 1H), 7.48 (d, J = 9.2 Hz, 1H), 7.16 (d, J = 2.0 Hz, 1H), 7.11 (dd, J = 8.8 Hz, 2.0 Hz, 1H), 6.14 (q, J = 6.4 Hz, 1H), 4.30 (d, J = 8.8 Hz, 2H), 3.99 (d, J = 8.8 Hz, 2H), 2.69- 2.59 (m, 4H), 1.79-1.70 (m, 7H), 1.45 (s, 3H) |
| Example 18 (R)-N-((1-(3-cyano-5- (5-(1-(3,5- dichloropyridin-4- yl)ethoxy)-1H-indazol- 3-yl)pyridin-2-yl)-3- methylazetidin-3- yl)methyl)methane- sulfonamide | 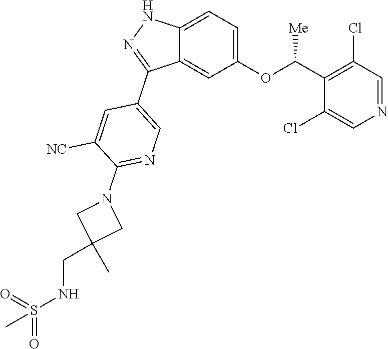 [SMILES: C[C@@H](Oc1ccc2[nH]nc(-c3cnc(N4CC(C)(CNS(C)(=O)=O)C4)c(C#N)c3)c2c1)c1c(Cl)cncc1Cl] | LCMS m/z = 586.2 (M + H); 1H NMR (400 MHz, DMSO-D6) δ 13.17 (s, 1H), 8.76 (d, J = 1.6 Hz, 1H), 8.57 (s, 2H), 8.13 (d, J = 1.6 Hz, 1H), 7.48 (d, J = 9.2 Hz, 1H), 7.36 (t, J = 6.4 Hz, 1H), 7.16 (s, 1H), 7.10 (dd, J = 9.2 Hz, 2.0 Hz, 1H), 6.14 (q, J = 6.4 Hz, 1H), 4.20 (d, J = 8.8 Hz, 2H), 3.93 (d, J = 8.8 Hz, 2H), 3.16 (d, J = 6.4 Hz, 2H), 2.94 (s, 3H), 1.76 (d, J = 6.4 Hz, 3H), 1.33 (s, 3H) |
| Example 19 2-[3- (cyclobutylamino)-3- methyl-azetidin-1-yl]- 5-[5-[(1R)-1-(3,5- dichloro-2-methyl-4- pyridyl)ethoxy]-1H- indazol-3-yl]pyridine- 3-carbonitrile | 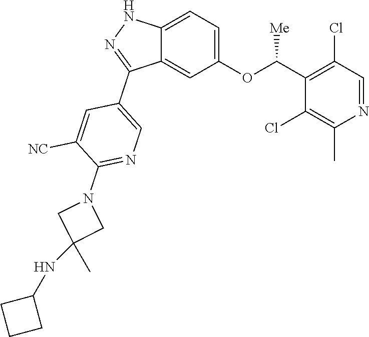 [SMILES: Cc1ncc(Cl)c([C@@H](C)Oc2ccc3[nH]nc(-c4cnc(N5CC(C)(NC6CCC6)C5)c(C#N)c4)c3c2)c1Cl] | LCMS m/z = 562.3 (M + H); 1H NMR (400 MHz, DMSO-d6) δ = 13.17 (br s, 1H), 9.62 (br s, 2H), 8.68 (d, J = 2.3 Hz, 1H), 8.36 (s, 1H), 8.21 (d, J = 2.3 Hz, 1H), 7.43 (d, J = 8.9 Hz, 1H), 7.06-7.02 (m, 2H), 6.10-6.04 (m, 1H), 4.39 (br d, J = 9.8 Hz, 2H), 4.22 (dd, J = 1.9, 9.8 Hz, 2H), 3.94-3.83 (m, 1H), 2.50 (s, 3H), 2.27-2.16 (m, 4H), 1.82-1.72 (m, 2H), 1.69 (d, J = 6.6 Hz, 3H), 1.57 (s, 3H) |
| Example 20 5-(5-((R)-1-(3,5- dichloropyridin-4- yl)ethoxy)-1H-indazol- 3-yl)-2-(3-methyl-3- (((S)-tetrahydrofuran- 3-yl)amino)azetidin-1- yl)nicotinonitrile | 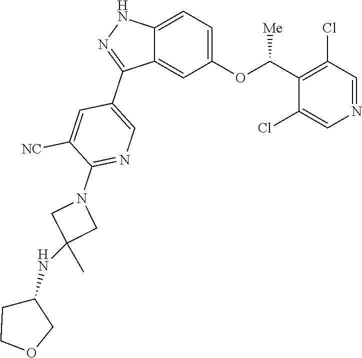 [SMILES: C[C@@H](Oc1ccc2[nH]nc(-c3cnc(N4CC(C)(N[C@H]5CCOC5)C4)c(C#N)c3)c2c1)c1c(Cl)cncc1Cl] | LCMS m/z = 564.3 (M + H); 1H NMR (400 MHz, DMSO-d6) δ 13.17 (brs, 1H), 8.76 (d, J = 2.0 Hz, 1H), 8.58 (s, 2H), 8.14 (d, J = 2.0 Hz, 1H), 7.48 (d, J = 9.2 Hz, 1H), 7.16 (d, J = 2.0 Hz, 1H), 7.11 (dd, J = 8.8 Hz, 2.4 Hz, 1H), 6.14 (q, J = 6.8 Hz, 1H), 4.08 (d, J = 8.8 Hz, 4H), 3.85-3.73 (m, 2H), 3.69-3.63 (m, 1H), 3.40-3.35 (m, 1H), 3.29- 3.25 (m, 1H), 2.10-1.90 (m, 1H), 1.76 (d, J = 6.4 Hz, 3H), 1.66-1.57 (m, 1H), 1.46 (s, 3H) |
| Example 21 (R)-5-(5-(1-(3,5- dichloropyridin-4- yl)ethoxy)-1H-indazol- 3-yl)-2-(3-((3,3- difluoropropyl)amino)- 3-methylazetidin-1- yl)nicotinonitrile | 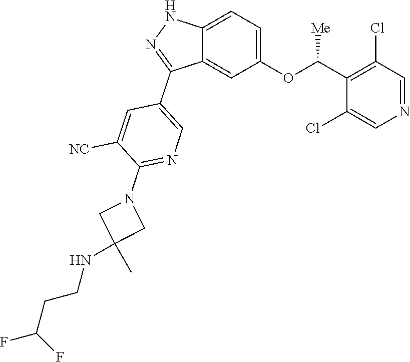 [SMILES: C[C@@H](Oc1ccc2[nH]nc(-c3cnc(N4CC(C)(NCCC(F)F)C4)c(C#N)c3)c2c1)c1c(Cl)cncc1Cl] | LCMS m/z = 573 (M + H); 1H NMR (400 MHz, DMSO-d6) δ1 3.17 (s, 1H), 8.77 (d, J = 2.8 Hz, 1H), 8.58 (s, 2H), 8.14 (d, J = 2.4 Hz, 1H), 7.50 (d, J = 8.8 Hz, 1H), 7.16 (d, J = 2.0 Hz, 1H), 7.12 (dd, J = 9.2 Hz, 2.4 Hz, 1H), 6.32-6.01 (m, 2H), 4.13 (d, J = 8.8 Hz, 2H), 4.06 (d, J = 8.4 Hz, 2H), 2.67-2.64 (m, 2H), 2.04-1.91 (m, 2H), 1.77 (d, J = 6.8 Hz, 3H), 1.43 (s, 3H) |
| Example 22 (R)-5-(5-(1-(3,5- dichloropyridin-4- yl)ethoxy)-1H-indazol- 3-yl)-2-(3-((2,2- difluoroethyl)amino)-3- methylazetidin-1- yl)nicotinonitrile | 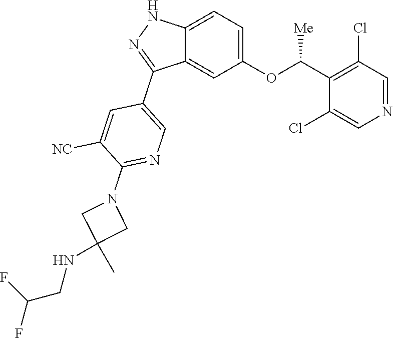 [SMILES: C[C@@H](Oc1ccc2[nH]nc(-c3cnc(N4CC(C)(NCC(F)F)C4)c(C#N)c3)c2c1)c1c(Cl)cncc1Cl] | LCMS m/z = 558.2 (M + H); 1H NMR (400 MHz, DMSO-d6) δ 13.17 (s, 1H), 8.77 (d, J = 2.4 Hz, 1H), 8.58 (s, 2H), 8.14 (d, J = 2.4 Hz, 1H), 7.50 (d, J = 8.8 Hz, 1H), 7.16 (d, J = 1.2 Hz, 1H), 7.12 (dd, J = 8.8 Hz, 2.4 Hz, 1H), 6.17-5.84 (m, 2H), 4.14 (d, J = 8.8 Hz, 2H), 4.06 (d, J = 8.8 Hz, 2H), 2.99-2.92 (m, 2H), 1.77 (d, J = 6.4 Hz, 3H), 1.45 (s, 3H). |
| Example 23 (R)-N-((1-(3-cyano-5- (5-(1-(3,5- dichloropyridin-4- yl)ethoxy)-1H-indazol- 3-yl)pyridin-2-yl)-3- methylazetidin-3- yl)methyl)-N- methylmethanesulfona- mide | 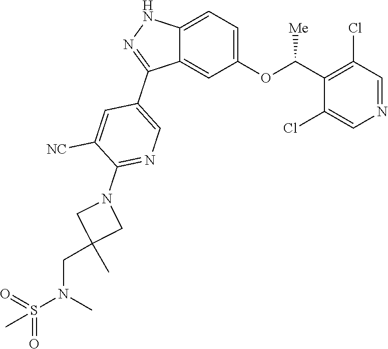 [SMILES: C[C@@H](Oc1ccc2[nH]nc(-c3cnc(N4CC(C)(CN(C)S(C)(=O)=O)C4)c(C#N)c3)c2c1)c1c(Cl)cncc1Cl] | LCMS m/z = 600.2 (M + H); 1H NMR (400 MHz, DMSO-d6) δ 13.17 (s, 1H), 8.77 (d, J = 2.4 Hz, 1H), 8.57 (s, 2H), 8.14 (d, J = 2.0 Hz, 1H), 7.48 (d, J = 8.8 Hz, 1H), 7.16 (d, J = 2.0 Hz, 1H), 7.10 (dd, J = 9.2 Hz, 2.4 Hz, 1H), 6.13 (q, J = 6.4 Hz, 1H), 4.24 (d, J = 8.4 Hz, 2H), 3.98 (d, J = 8.0 Hz, 2H), 3.29- 3.25 (m, 2H), 2.92 (s, 3H), 2.84 (s, 3H), 1.76 (d, J = 6.4 Hz, 3H), 1.40 (s, 3H) |
| Example 24 5-(5-((R)-1-(3,5- dichloropyridin-4- yl)ethoxy)-6-methoxy- 1H-indazol-3-yl)-2-(3- methyl-3-(((S)- tetrahydrofuran-3- yl)amino)azetidin-1- yl)nicotinonitrile | 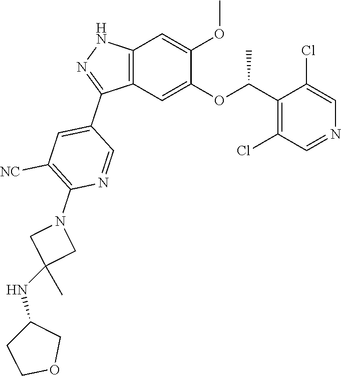 [SMILES: COc1cc2[nH]nc(-c3cnc(N4CC(C)(N[C@H]5CCOC5)C4)c(C#N)c3)c2cc1O[C@H](C)c1c(Cl)cncc1Cl] | LCMS m/z = 594.3 (M + H); 1H NMR (400 MHz, DMSO-d6) δ 12.97 (s, 1H), 8.73 (s, 1H), 8.59 (s, 2H), 8.07 (s, 1H), 7.04 (s, 1H), 7.01 (s, 1H), 6.00 (q, J = 6.8 Hz, 1H), 4.12-4.07 (m, 4H), 3.86 (s, 3H), 3.83-3.79 (m, 2H), 3.69-3.64 (m, 1H), 3.41-3.39 (m, 1H), 3.36-3.29 (m, 1H), 2.11-2.02 (m, 1H), 1.76 (d, J = 6.8 Hz, 3H), 1.67-1.56 (m, 1H), 1.46 (s, 3H) |
| Example 25 5-(5-((R)-1-(3,5- dichloropyridin-4- yl)ethoxy)-6-methoxy- 1H-indazol-3-yl)-2-(3- methyl-3-(((R)- tetrahydrofuran-3- yl)amino)azetidin-1- yl)nicotinonitrile | 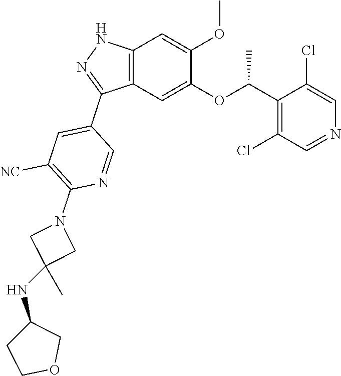 [SMILES: COc1cc2[nH]nc(-c3cnc(N4CC(C)(N[C@@H]5CCOC5)C4)c(C#N)c3)c2cc1O[C@H](C)c1c(Cl)cncc1Cl] | LCMS m/z = 594.3 (M + H); 1H NMR (400 MHz, DMSO-d6) δ 12.96 (s, 1H), 8.72 (d, J = 2.0 Hz, 1H), 8.59 (s, 2H), 8.06 (d, J = 2.0 Hz, 1H), 7.04 (s, 1H), 7.00 (s, 1H), 6.00 (q, J = 6.4 Hz, 1H), 4.10-4.01 (m, 4H), 3.86 (s, 3H), 3.82-3.79 (m, 2H), 3.69-3.65 (m, 1H), 3.32-3.30 (m, 1H), 3.30-3.28 (m, 1H), 2.10- 2.01 (m, 1H), 1.76 (d, J = 6.4 Hz, 3H), 1.65-1.57 (m, 1H), 1.46 (s, 3H) |
| Example 26 (R)-5-(5-(1-(3,5- dichloropyridin-4- yl)ethoxy)-6-methoxy- 1H-indazol-3-yl)-2-(3′- methyl-[1,3′- biazetidin]-1′- yl)nicotinonitrile | 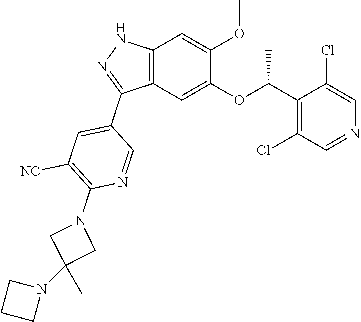 [SMILES: COc1cc2[nH]nc(-c3cnc(N4CC(C)(N5CCC5)C4)c(C#N)c3)c2cc1O[C@H](C)c1c(Cl)cncc1Cl] | LCMS m/z = 564.3 (M + H); 1H NMR (400 MHz, DMSO-d6) δ 12.97 (s, 1H), 8.72 (d, J = 2.4 Hz, 1H), 8.59 (s, 2H), 8.08 (d, J = 2.4 Hz, 1H), 7.04 (s, 1H), 7.01 (s, 1H), 6.00 (q, J = 7.2 Hz, 1H), 4.32 (d, J = 9.2 Hz, 2H), 3.93 (d, J = 8.8 Hz, 2H), 3.86 (s, 3H), 3.30-3.27 (m, 4H), 1.98-1.95 (m, 2H), 1.76 (d, J = 6.8 Hz, 3H), 1.29 (s, 3H) |
| Example 27 (R)-2-(3- ((cyclopropylmethyl)a- mino)-3- methylazetidin-1-yl)-5- (5-(1-(3,5- dichloropyridin-4- yl)ethoxy)-1H-indazol- 3-yl)nicotinonitrile | 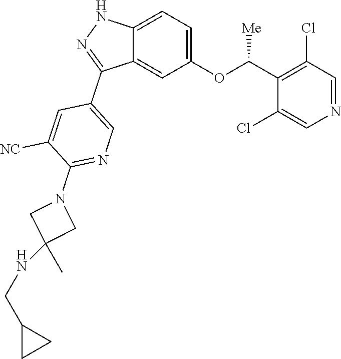 [SMILES: C[C@@H](Oc1ccc2[nH]nc(-c3cnc(N4CC(C)(NCC5CC5)C4)c(C#N)c3)c2c1)c1c(Cl)cncc1Cl] | LCMS m/z = 548.3 (M + H); 1H NMR (400 MHz, DMSO-d6) δ 13.17 (s, 1H), 8.76 (d, J = 2.4 Hz, 1H), 8.57 (s, 2H), 8.12 (d, J = 2.4 Hz, 1H), 7.48 (d, J = 8.8 Hz, 1H), 7.15 (d, J = 2.0 Hz, 1H), 7.11 (dd, J = 8.8 Hz, 2.0 Hz, 1H), 6.14 (q, J = 6.8 Hz, 1H), 4.14 (d, J = 8.8 Hz, 2H), 4.03 (d, J = 8.4 Hz, 2H), 2.41 (d, J = 7.2 Hz, 2H), 1.76 (d, J = 6.4 Hz, 3H), 1.42 (s, 3H), 0.90-0.83 (m, 1H), 0.44-0.38 (m, 2H), 0.17-0.13 (m, 2H) |
| Example 28 (R)-5-(5-(1-(3,5- dichloropyridin-4- yl)ethoxy)-1H-indazol- 3-yl)-2-(3′-methyl- [1,3′-biazetidin]-1′- yl)nicotinonitrile | 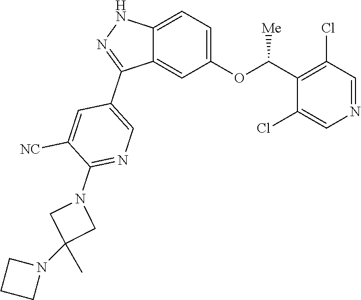 [SMILES: C[C@@H](Oc1ccc2[nH]nc(-c3cnc(N4CC(C)(N5CCC5)C4)c(C#N)c3)c2c1)c1c(Cl)cncc1Cl] | LCMS m/z = 534.2 (M + H); 1H NMR (400 MHz, DMSO-d6) δ 13.17 (s, 1H), 8.77 (d, J = 2.0 Hz, 1H), 8.58 (s, 2H), 8.15 (d, J = 2.0 Hz, 1H), 7.49 (d, J = 9.2 Hz, 1H), 7.16 (s, 1H), 7.10 (dd, J = 9.2 Hz, 2.0 Hz, 1H), 6.14 (q, J = 6.8 Hz, 1H), 4.32 (d, J = 9.6 Hz, 2H), 3.94 (d, J = 9.2 Hz, 2H), 3.29-3.24 (m, 4H), 2.02-1.94 (m, 2H), 1.76 (d, J = 7.2 Hz, 3H), 1.30 (s, 3H) |
| Example 29 (R)-5-(5-(1-(3,5- dichloropyridin-4- yl)ethoxy)-1H-indazol- 3-yl)-2-(3- (dimethylamino)-3- methylazetidin-1- yl)nicotinonitrile | 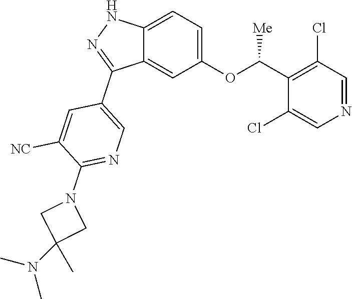 | LCMS m/z = 552.2 (M + H); 1H NMR (400 MHz, DMSO-d6) δ 13.18 (s, 1H), 8.76 (d, J = 2.0 Hz, 1H), 8.58 (s, 2H), 8.15 (d, J = 2.0 Hz, 1H), 7.48 (d, J = 9.2 Hz, 1H), 7.18-7.09 (m, 2H), 6.14 (q, J = 6.8 Hz, 1H), 4.08 (d, J = 8.0 Hz, 2H), 3.98 (d, J = 8.4 Hz, 2H), 2.15 (s, 6H), 1.76 (d, J = 6.8 Hz, 3H), 1.32 (s, 3H) |
| Example 30 5-[5-[(1R)-1-(3,5- dichloro-2-methyl-4- pyridyl)ethoxy]-1H- indazol-3-yl]-2-[3- methyl-3-(oxetan-3- ylamino)azetidin-1- yl]pyridine-3- carbonitrile | 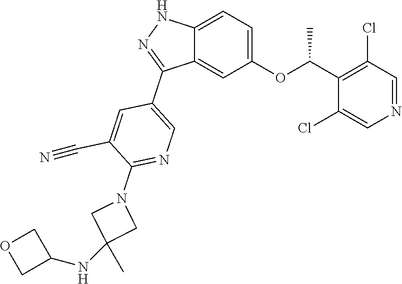 | LCMS m/z = 564 (M + H); 1H NMR (400 MHz, DMSO-d6) δ = 13.16 (s, 1H), 8.68 (d, J = 2.3 Hz, 1H), 8.43 (s, 1H), 8.17 (d, J = 2.3 Hz, 1H), 7.47 (s, 1H), 7.12 - 7.07 (m, 2H), 6.18 - 6.09 (m, 1H), 4.67 (dd, J = 6.1, 7.1 Hz, 2H), 4.37 (t, J = 6.4 Hz, 2H), 4.15-3.94 (m, 6H), 2.56 (s, 3H), 1.76 (d, J = 6.6 Hz, 3H), 1.34 (s, 3H) |
| Example 31 5-[5-[(1R)-1-(3,5- dichloro-4- pyridyl)ethoxy]-1H- indazol-3-yl]-2-[3- (diethylamino)-3- methyl-azetidin-1- yl]pyridine-3- carbonitrile | 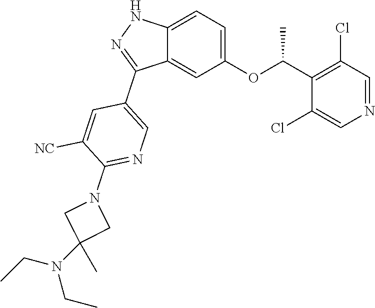 [SMILES: CCN(CC)C1(C)CN(c2ncc(-c3n[nH]c4ccc(O[C@H](C)c5c(Cl)cncc5Cl)cc34)cc2C#N)C1] | LCMS m/z = 550.2 (M + H); 1H NMR (400 MHz, DMSO-d6) δ = 13.17 (s, 1H), 8.77 (d, J = 2.3 Hz, 1H), 8.57 (s, 2H), 8.14 (d, J = 2.2 Hz, 1H), 7.49 (d, J = 9.0 Hz, 1H), 7.16 (d, J = 2.0 Hz, 1H), 7.11 (dd, J = 2.3, 9.0 Hz, 1H), 6.17-6.08 (m, 1H), 4.11-3.98 (m, 4H), 2.49- 2.41 (m, 4H), 1.76 (d, J = 6.7 Hz, 3H), 1.39 (s, 3H), 1.05 (t, J = 7.2 Hz, 6H) |
| Example 32 5-[5-[(1R)-1-(3,5- dichloro-2-methyl-4- pyridyl)ethoxy]-1H- indazol-3-yl]-2-[3- methyl-3- (methylamino)azetidin- 1-yl]pyridine-3- carbonitrile | 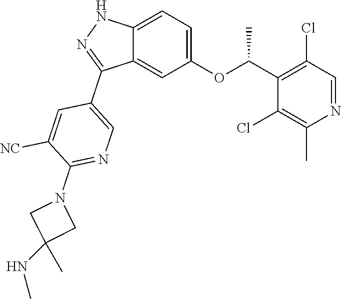 [SMILES: CNC1(C)CN(c2ncc(-c3n[nH]c4ccc(O[C@H](C)c5c(Cl)cnc(C)c5Cl)cc34)cc2C#N)C1] | LCMS m/z = 523.4 (M + H); 1H NMR (400 MHz, DMSO-d6) δ = 13.24 (s, 1H), 9.28 (br s, 2H), 8.75 (d, J = 2.2 Hz, 1H), 8.43 (s, 1H), 8.28 (d, J = 2.2 Hz, 1H), 7.50 (d, J = 8.9 Hz, 1H), 7.14-7.09 (m, 2H), 6.17-6.11 (m, 1H), 4.47 (d, J = 10.0 Hz, 2H), 4.28 (dd, J = 2.3, 9.9 Hz, 2H), 2.67 (s, 3H), 2.57 (s, 3H), 1.76 (d, J = 6.6 Hz, 3H), 1.65 (s, 3H) |
| Example 33 5-(5-((R)-1-(3,5- dichloropyridin-4- yl)ethoxy)-1H-indazol- 3-yl)-2-(3-methyl-3- (((R)-tetrahydrofuran- 3-yl)amino)azetidin-1- yl)nicotinonitrile | 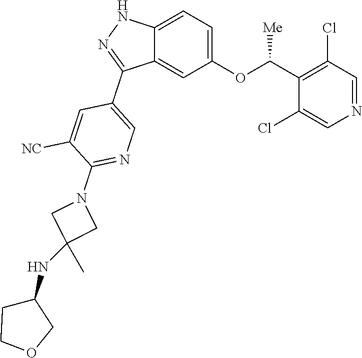 [SMILES: C[C@@H](Oc1ccc2[nH]nc(-c3cnc(N4CC(C)(N[C@@H]5CCOC5)C4)c(C#N)c3)c2c1)c1c(Cl)cncc1Cl] | LCMS m/z = 564.3 (M + H); 1H NMR (400 MHz, DMSO-d6) δ 13.16 (s, 1H), 8.77 (d, J = 2.0 Hz, 1H), 8.58 (s, 2H), 8.14 (d, J = 2.4 Hz, 1H), 7.48 (d, J = 9.2 Hz, 1H), 7.16 (d, J = 2.4 Hz, 1H), 7.10 (dd, J = 8.8 Hz, 2.0 Hz, 1H), 6.14 (q, J = 6.4 Hz, 1H), 4.10-4.08 (m, 4H), 3.85-3.63 (m, 3H), 3.46-3.35 (m, 2H), 2.09-2.04 (m, 1H), 1.76 (d, J = 6.4Hz, 3H), 1.67-1.55 (m, 1H), 1.47 (s, 3H) |
| Example 34 (R)-5-(5-(1-(3,5- dichloropyridin-4- yl)ethoxy)-1H-indazol- 3-yl)-2-(3-methyl-3- morpholinoazetidin-1- yl)nicotinonitrile | 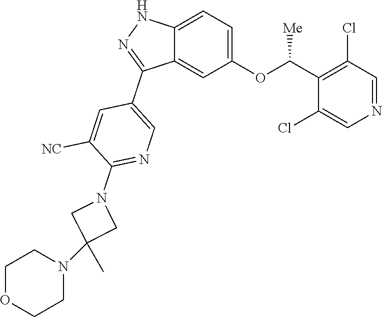 [SMILES: C[C@@H](Oc1ccc2[nH]nc(-c3cnc(N4CC(C)(N5CCOCC5)C4)c(C#N)c3)c2c1)c1c(Cl)cncc1Cl] | LCMS m/z = 564.3 (M + H); 1H NMR (400 MHz, DMSO-d6) δ 13.18 (s, 1H), 8.76 (d, J = 2.4 Hz, 1H), 8.57 (s, 2H), 8.15 (d, J = 1.6 Hz, 1H), 7.49 (d, J = 8.8 Hz, 1H), 7.15 (s, 1H), 7.10 (dd, J = 9.2 Hz, 2.0 Hz, 1H), 6.14 (q, J = 6.4 Hz, 1H), 4.12 (d, J = 8.8 Hz, 2H), 3.98 (d, J = 8.4 Hz, 2H), 3.67-3.57 (m, 4H), 2.47-2.40 (m, 4H), 1.76 (d, J = 6.4 Hz, 3H), 1.35 (s, 3H) |
| Example 35 (R)-5-(5-(1-(3,5- dichloropyridin-4- yl)ethoxy)-1H-indazol- 3-yl)-2-(3- (ethylamino)-3- methylazetidin-1- yl)nicotinonitrile | 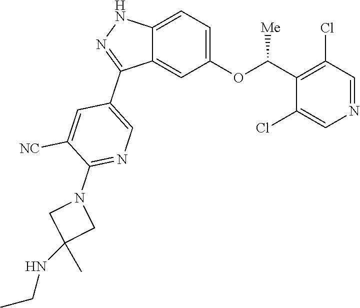 | LCMS m/z = 552.3 (M + H); 1H NMR (400 MHz, DMSO-d6) δ 13.17 (s, 1H), 8.77 (d, J = 2.4 Hz, 1H), 8.57 (s, 2H), 8.14 (d, J = 2.0 Hz, 1H), 7.49 (d, J = 9.2 Hz, 1H), 7.16 (d, J = 2.4 Hz, 1H), 7.10 (dd, J = 8.8, 2.0 Hz, 1H), 6.14 (q, J = 6.4 Hz, 1H), 4.17 (d, J = 8.8 Hz, 2H), 4.07 (d, J = 8.8 Hz, 2H), 2.62 (q, J = 7.2 Hz, 2H), 1.76 (d, J = 6.8 Hz, 3H), 1.45 (s, 3H), 1.07 (t, J = 7.2 Hz, 3H) |
| Example 36 5-[5-[(1R)-1-(3,5- dichloro-2-methyl-4- pyridyl)ethoxy]-1H- indazol-3-yl]-2-[3- (diethylamino)-3- methyl-azetidin-1- yl]pyridine-3- carbonitrile | 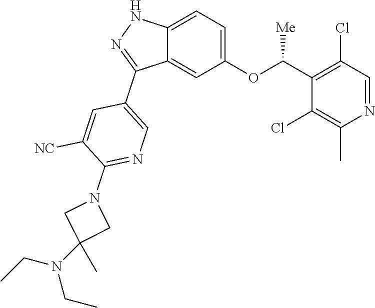 [SMILES: CCN(CC)C1(C)CN(c2ncc(-c3n[nH]c4ccc(O[C@H](C)c5c(Cl)cnc(C)c5Cl)cc34)cc2C#N)C1] | LCMS m/z = 564.2 (M + H); 1H NMR (400 MHz, DMSO-d6) δ = 13.18 (s, 1H), 8.70 (d, J = 2.3 Hz, 1H), 8.42 (s, 1H), 8.16 (d, J = 2.2 Hz, 1H), 7.49 (d, J = 9.0 Hz, 1H), 7.10 (d, J = 9.3 Hz, 1H), 7.08 (s, 1H), 6.12 (q, J = 6.6 Hz, 1H), 4.13- 3.95 (m, 4H), 2.58-2.53 (m, 3H), 2.49-2.30 (m, 4H), 1.75 (d, J = 6.7 Hz, 3H), 1.38 (s, 3H), 1.04 (br t, J = 7.1 Hz, 6H) |
| Example 37 5-[5-[(1R)-1-(3,5- dichloro-4- pyridyl)ethoxy]-6- methoxy-1H-indazol-3- yl]-2-[3-(2- methoxyethylamino)-3- methyl-azetidin-1- yl]pyridine-3- carbonitrile | 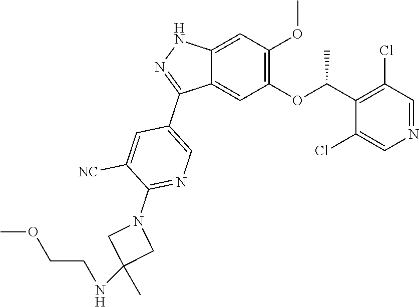 [SMILES: COCCNC1(C)CN(c2ncc(-c3n[nH]c4cc(OC)c(O[C@H](C)c5c(Cl)cncc5Cl)cc34)cc2C#N)C1] | LCMS m/z = 582.2 (M + H); 1H NMR (400 MHz, DMSO-d6) δ = 12.96 (br s, 1H), 8.71 (d, J = 2.3 Hz, 1H), 8.59 (s, 2H), 8.05 (d, J = 2.3 Hz, 1H), 7.04 (s, 1H), 7.01 (s, 1H), 5.99 (q, J = 6.7 Hz, 1H), 4.11 (d, J = 8.8 Hz, 2H), 4.03 (d, J = 8.7 Hz, 2H), 3.86 (s, 3H), 3.44-3.34 (m, 2H), 3.28-3.25 (m, 3H), 2.70 (q, J = 6.1 Hz, 2H), 2.48-2.27 (m, 1H), 1.76 (d, J = 6.6 Hz, 3H), 1.45- 1.36 (m, 3H) |
| Example 38 5-[5-[(1R)-1-(3,5- dichloro-2-methyl-4- pyridyl)ethoxy]-1H- indazol-3-yl]-2-(3- methyl-3-morpholino- azetidin-1-yl)pyridine- 3-carbonitrile | 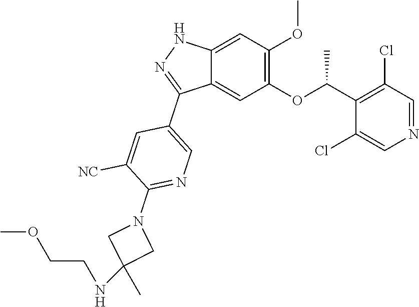 | LCMS m/z = 578.1 (M + H); 1H NMR (400 MHz, CDCl3) δ = 9.98 (br s, 1H), 8.75 (d, J = 2.2 Hz, 1H), 8.37 (s, 1H), 8.09 (d, J = 2.3 Hz, 1H), 7.39 (d, J = 9.0 Hz, 1H), 7.17 (dd, J = 2.2, 9.0 Hz, 1H), 7.08 (d, J = 2.2 Hz, 1H), 6.09 (q, J = 6.7 Hz, 1H), 4.26 (d, J = 8.6 Hz, 2H), 4.09 (br d, J = 7.6 Hz, 2H), 3.81-3.75 (m, 4H), 2.63 (s, 3H), 2.56-2.51 (m, 4H), 1.82 (s, 3H), 1.46 (s, 3H) |
| Example 39 2-(3-amino-3-methyl- azetidin-1-yl)-5-[5- [(1R)-1-(3,5-dichloro- 2-methyl-4- pyridyl)ethoxy]-1H- indazol-3-yl]pyridine- 3-carbonitrile | 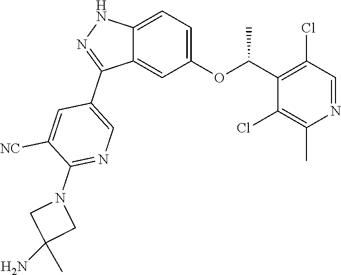 [SMILES: Cc1ncc(Cl)c([C@@H](C)Oc2ccc3[nH]nc(-c4cnc(N5CC(C)(N)C5)c(C#N)c4)c3c2)c1Cl] | LCMS m/z = 508.2 (M + H); 1H NMR (400 MHz, DMSO-d6) δ = 8.70-8.61 (m, 1H), 8.42 (s, 1H), 8.18-8.07 (m, 1H), 7.52 (br d, J = 9.3 Hz, 1H), 7.09-7.06 (m, 2H), 6.12 (q, J = 6.6 Hz, 1H), 4.50 (br d, J = 8.6 Hz, 1H), 4.14-4.01 (m, 4H), 3.98 (br d, J = 8.7 Hz, 1H), 2.57-2.55 (m, 3H), 2.41-2.01 (m, 2H), 1.75 (d, J = 6.6 Hz, 3H), 1.50 (s, 1H), 1.42 (s, 3H) |
| Example 40 5-[5-[(1R)-1-(3,5- dichloro-2-methyl-4- pyridyl)ethoxy]-1H- indazol-3-yl]-2-[3- (ethylamino)-3-methyl- azetidin-1-yl]pyridine- 3-carbonitrile | 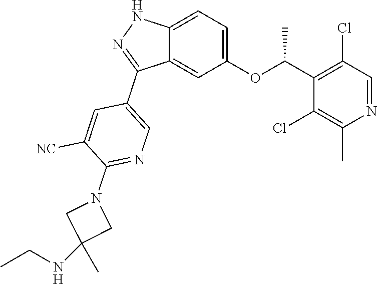 [SMILES: CCNC1(C)CN(c2ncc(-c3n[nH]c4ccc(O[C@H](C)c5c(Cl)cnc(C)c5Cl)cc34)cc2C#N)C1] | LCMS m/z = 536.2 (M + H); 1H NMR (400 MHz, DMSO-d6) δ = 13.15 (s, 1H), 8.68 (d, J = 2.3 Hz, 1H), 8.43 (s, 1H), 8.16 (d, J = 2.3 Hz, 1H), 7.48 (d, J = 8.9 Hz, 1H), 7.09 (s, 1H), 7.10 (d, J = 9.4 Hz, 2H), 6.16-6.10 (m, 1H), 4.12 (d, J = 8.7 Hz, 2H), 4.03 (d, J = 8.7 Hz, 2H), 2.60-2.52 (m, 5H), 1.76 (d, J = 6.6 Hz, 3H), 1.42 (s, 3H), 1.04 (t, J = 7.1 Hz, 3H) |
| Example 41 5-[5-[(1R)-1-(3,5- dichloro-4- pyridyl)ethoxy]-6- methoxy-1H-indazol-3- yl]-2-[3-(3,3- difluoropropylamino)- 3-methyl-azetidin-1- yl]pyridine-3- carbonitrile | 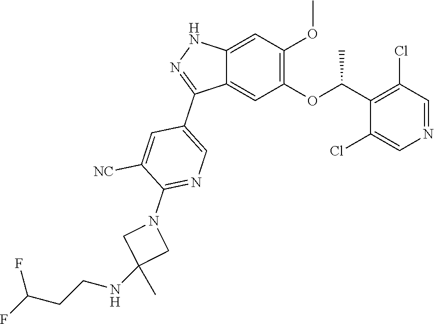 [SMILES: COc1cc2[nH]nc(-c3cnc(N4CC(C)(NCCC(F)F)C4)c(C#N)c3)c2cc1O[C@H](C)c1c(Cl)cncc1Cl] | LCMS m/z = 602.2 (M + H); 1H NMR (400 MHz, DMSO-d6) δ = 12.96 (s, 1H), 8.73 (d, J = 2.3 Hz, 1H), 8.59 (s, 2H), 8.08 (s, 1H), 7.04 (s, 1H), 7.01 (s, 1H), 6.36-5.93 (m, 2H), 4.10 (br d, J = 19.7 Hz, 4H), 3.86 (s, 3H), 2.76-2.61 (m, 2H), 2.33 (td, J = 1.8, 3.7 Hz, 1H), 2.10-1.93 (m, 2H), 1.76 (d, J = 6.6 Hz, 3H), 1.44 (br s, 3H) |
| Example 42 5-[5-[(1R)-1-(3,5- dichloro-4- pyridyl)ethoxy]-6- methoxy-1H-indazol-3- yl]-2-[3-(2,2- difluoroethylamino)-3- methyl-azetidin-1- yl]pyridine-3- carbonitrile | 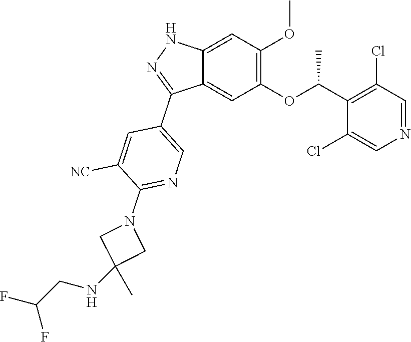 [SMILES: COc1cc2[nH]nc(-c3cnc(N4CC(C)(NCC(F)F)C4)c(C#N)c3)c2cc1O[C@H](C)c1c(Cl)cncc1Cl] | LCMS m/z = 588 (M + H); 1H NMR (400 MHz, DMSO-d6) δ = 1H NMR (400 MHz, DMSO-d6) δ = 12.96 (s, 1H), 8.72 (d, J = 2.3 Hz, 1H), 8.59 (s, 2H), 8.07 (d, J = 2.2 Hz, 1H), 7.04 (s, 1H), 7.01 (s, 1H), 6.13-5.85 (m, 2H), 4.14-4.10 (m, 2H), 4.06-4.02 (m, 2H), 3.86 (s, 3H), 3.00-2.90 (m, 2H), 2.83 (br d, J = 7.6 Hz, 1H), 1.76 (d, J = 6.6 Hz, 3H), 1.44 (s, 3H) |
| Example 43 (R)-4-(1-(5-(5-(1-(3,5- dichloropyridin-4- yl)ethoxy)-1H-indazol- 3-yl)pyrimidin-2-yl)-3- methylazetidin-3- yl)morpholine | 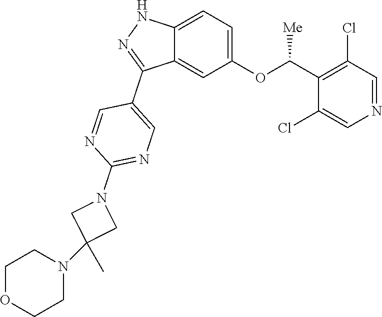 [SMILES: C[C@@H](Oc1ccc2[nH]nc(-c3cnc(N4CC(C)(N5CCOCC5)C4)nc3)c2c1)c1c(Cl)cncc1Cl] | LCMS m/z = 540.3 (M + H); 1H NMR (400 MHz, DMSO-d6) δ 13.13 (brs, 1H), 8.78 (s, 2H), 8.57 (s, 2H), 7.47 (d, J = 9.2 Hz, 1H), 7.23 (d, J = 2.0 Hz, 1H), 7.10 (dd, J = 8.8 Hz, 2.4 Hz, 1H), 6.15 (q, J = 6.4 Hz, 1H), 3.94 (d, J = 8.4 Hz, 2H), 3.76 (d, J = 8.4 Hz, 2H), 3.61 (t, J = 4.0 Hz, 4H), 2.44 (t, J = 4.0 Hz, 4H), 1.75 (d, J = 6.4 Hz, 3H), 1.32 (s, 3H) |
| Example 44 (R)-1-(5-(5-(1-(3,5- dichloropyridin-4- yl)ethoxy)-1H-indazol- 3-yl)pyrimidin-2-yl)- N,N,3- trimethylazetidin-3- amine | 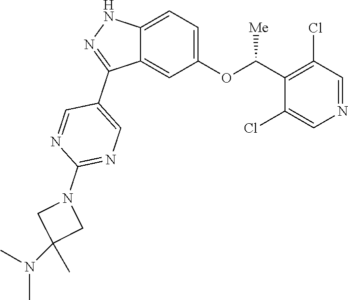 [SMILES: C[C@@H](Oc1ccc2[nH]nc(-c3cnc(N4CC(C)(N(C)C)C4)nc3)c2c1)c1c(Cl)cncc1Cl] | LCMS m/z = 498.2 (M + H); 1H NMR (400 MHz, DMSO-d6) δ 13.13 (s, 1H), 8.75 (s, 2H), 8.57 (s, 2H), 7.47 (d, J = 9.2 Hz, 1H), 7.24 (d, J = 2.0 Hz, 1H), 7.10 (dd, J = 9.2 Hz, 2.4 Hz, 1H), 6.15 (q, J = 6.8 Hz, 1H), 3.90 (d, J = 8.4 Hz, 2H), 3.76 (d, J = 8.4 Hz, 2H), 2.13 (s, 6H), 1.75 (d, J = 6.8 Hz, 3H), 1.29 (s, 3H) |
| Example 45 5-[5-[(1R)-1-(3,5- dichloro-2-methyl-4- pyridyl)ethoxy]-1H- indazol-3-yl]-2-[3- (dimethylamino)-3- methyl-azetidin-1- yl]pyridine-3- carbonitrile | 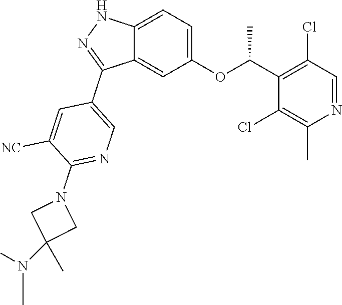 [SMILES: Cc1ncc(Cl)c([C@@H](C)Oc2ccc3[nH]nc(-c4cnc(N5CC(C)(N(C)C)C5)c(C#N)c4)c3c2)c1Cl] | LCMS m/z = 536.1 (M + H); 1H NMR (400 MHz, DMSO-d6) δ = 13.17 (s, 1H), 8.69 (d, J = 2.3 Hz, 1H), 8.43 (s, 1H), 8.17 (d, J = 2.3 Hz, 1H), 7.50 (s, 1H), 7.13-7.08 (m, 2H), 6.13 (q, J = 6.6 Hz, 1H), 4.08 (d, J = 8.7 Hz, 2H), 3.97 (d, J = 8.6 Hz, 2H), 2.56 - 2.54 (m, 3H), 2.14 (s, 6H), 1.75 (d, J = 6.7 Hz, 3H), 1.32 (s, 3H) |
| Example 46 2-[3-(cyclobutoxy)-3- methyl-azetidin-1-yl]- 5-[5-[(1R)-1-(3,5- dichloro-2-methyl-4- pyridyl)ethoxy]-1H- indazol-3-yl]pyridine- 3-carbonitrile | 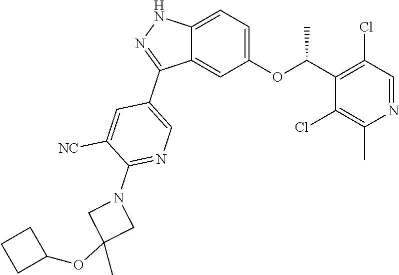 [SMILES: Cc1ncc(Cl)c([C@@H](C)Oc2ccc3[nH]nc(-c4cnc(N5CC(C)(OC6CCC6)C5)c(C#N)c4)c3c2)c1Cl] | LCMS m/z = 563.2 (M + H); 1H NMR (400 MHz, DMSO-d6) δ = 13.18 (s, 1H), 8.70 (d, J = 2.2 Hz, 1H), 8.41 (s, 1H), 8.16 (d, J = 2.2 Hz, 1H), 7.49 (d, J = 8.9 Hz, 1H), 7.11 (dd, J = 2.3, 9.0 Hz, 1H), 7.07 (s, 1H), 6.10 (q, J = 6.7 Hz, 1H), 4.20-3.98 (m, 5H), 2.58-2.53 (m, 3H), 2.26-2.10 (m, 2H), 2.02- 1.84 (m, 2H), 1.75 (d, J = 6.7 Hz, 3H), 1.61 (q, J = 10.0 Hz, 1H), 1.50 (s, 3H), 1.49-1.39 (m, 1H) |
| Example 47 5-[5-[(1R)-1-(3,5- dichloro-4- pyridyl)ethoxy]-6- methoxy-1H-indazol-3- yl]-2-[3-(2- hydroxyethylamino)-3- methyl-azetidin-1- yl]pyridine-3- carbonitrile | 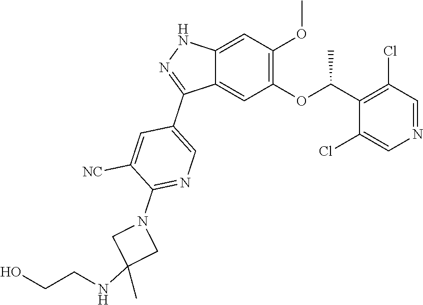 [SMILES: COc1cc2[nH]nc(-c3cnc(N4CC(C)(NCCO)C4)c(C#N)c3)c2cc1O[C@H](C)c1c(Cl)cncc1Cl] | LCMS m/z = 568.1 (M + H); 1H NMR (400 MHz, CDCl3) δ = 8.72 (br s, 1H), 8.48 (s, 2H), 7.93 (br s, 1H), 6.97 (s, 1H), 6.88 (s, 1H), 6.02- 5.89 (m, 1H), 4.31 (br d, J = 9.0 Hz, 2H), 4.23-4.15 (m, 2H), 3.92 (s, 3H), 3.80 (br s, 2H), 2.90 (br s, 5H), 1.84 (d, J = 6.6 Hz, 3H), 1.58 (s, 3H) |
| Example 48 (R)-5-(5-(1-(3,5- dichloropyridin-4- yl)ethoxy)-1H-indazol- 3-yl)-2-(3- (isopropylamino)-3- methylazetidin-1- yl)nicotinonitrile | 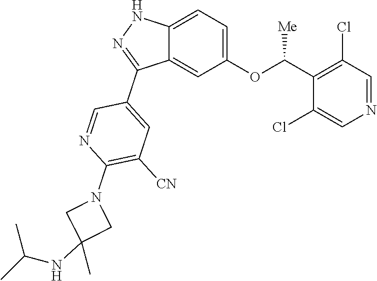 [SMILES: CC(C)NC1(C)CN(c2ncc(-c3n[nH]c4ccc(O[C@H](C)c5c(Cl)cncc5Cl)cc34)cc2C#N)C1] | LCMS m/z = 536.3 (M + H); 1H NMR (400 MHz, DMSO-d6) δ 13.18 (s, 1H), 8.76 (d, J = 2.4 Hz, 1H), 8.58 (s, 2H), 8.13 (d, J = 2.4 Hz, 1H), 7.49 (d, J = 8.8 Hz, 1H), 7.15 (s, 1H), 7.10 (dd, J = 8.8 Hz, 2.0 Hz, 1H), 6.14 (q, J = 6.8 Hz, 1H), 4.10-4.05 (m, 4H), 2.97-2.94 (m, 1H), 1.76 (d, J = 6.4 Hz, 3H), 1.47 (s, 3H), 1.00 (d, J = 6.4 Hz, 6H) |
| Example 49 (R)-5-(5-(1-(3,5- dichloropyridin-4- yl)ethoxy)-6-methoxy- 1H-indazol-3-y1)-2-(3- ((dimethylamino)methyl)- 3-methylazetidin-1- yl)nicotinonitrile | 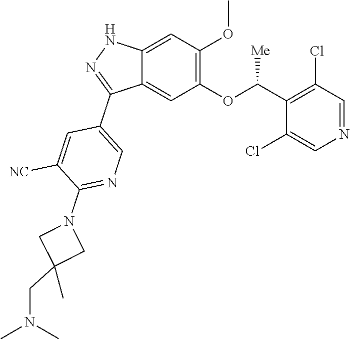 [SMILES: COc1cc2[nH]nc(-c3cnc(N4CC(C)(CN(C)C)C4)c(C#N)c3)c2cc1O[C@H](C)c1c(Cl)cncc1Cl] | LCMS m/z = 566.3 (M + H); 1H NMR (400 MHz, DMSO-d6) δ 12.97 (s, 1H), 8.71 (d, J = 2.8 Hz, 1H), 8.59 (s, 2H), 8.06 (d, J = 2.4 Hz, 1H), 7.04 (s, 1H), 7.01 (s, 1H), 6.00 (q, J = 6.8 Hz, 1H), 4.07 (d, J = 8.4 Hz, 2H), 3.98 (d, J = 8.8 Hz, 2H), 3.86 (s, 3H), 3.28-3.32 (m, 2H), 2.18 (s, 6H), 1.76 (d, J = 6.8 Hz, 3H), 1.38 (s, 3H) |
| Example 50 (R)-5-(5-(1-(3,5- dichloropyridin-4- yl)ethoxy)-6-methoxy- 1H-indazol-3-yl)-2-(3- ((isopropylamino)meth- yl)-3-methylazetidin-1- yl)nicotinonitrile | 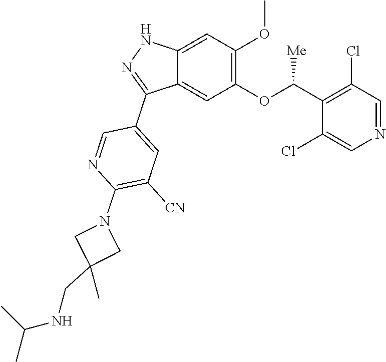 [SMILES: COc1cc2[nH]nc(-c3cnc(N4CC(C)(CNC(C)C)C4)c(C#N)c3)c2cc1O[C@H](C)c1c(Cl)cncc1Cl] | LCMS m/z = 580.3 (M + H); 1H NMR (400 MHz, DMSO-d6) δ 12.99 (s, 1H), 8.71 (d, J = 2.4 Hz, 1H), 8.59 (s, 2H), 8.06 (d, J = 2.4 Hz, 1H), 7.03 (s, 1H) 7.01 (s, 1H), 6.01 (q, J = 7.2 Hz, 1H), 4.17 (d, J = 8.4 Hz, 2H), 3.92 (d, J = 8.0 Hz, 2H), 3.86 (s, 3H), 3.21-3.32 (2H), 2.93-2.80 (m, 1H), 1.76 (d, J = 6.8 Hz, 3H), 1.35 (s, 3H), 1.10 (s, 6H) |
| Example 51 (R)-1-(5-(5-(1-(3,5- dichloropyridin-4- yl)ethoxy)-6-methoxy- 1H-indazol-3- yl)pyrimidin-2-yl)- N,N,3- trimethylazetidin-3- amine | 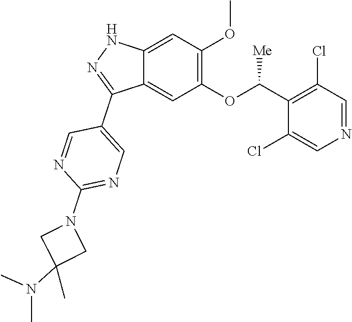 [SMILES: COc1cc2[nH]nc(-c3cnc(N4CC(C)(N(C)C)C4)nc3)c2cc1O[C@H](C)c1c(Cl)cncc1Cl] | LCMS m/z = 528.2 (M + H); 1H NMR (400 MHz, DMSO-d6) δ 12.94 (s, 1H), 8.70 (s, 2H), 8.59 (s, 2H), 7.13 (s, 1H), 7.00 (s, 1H), 5.99 (q, J = 6.0 Hz, 1H), 3.89 (d, J = 8.4 Hz, 2H), 3.85 (s, 3H), 3.76 (d, J = 8.4 Hz, 2H), 2.13 (s, 6H), 1.75 (d, J = 6.4 Hz, 3H), 1.29 (s, 3H) |
| Example 52 5-[5-[(1R)-1-(3,5- dichloro-4- pyridyl)ethoxy]-1H- indazol-3-yl]-2-[3-(2- hydroxyethylamino)-3- methyl-azetidin-1- yl]pyridine-3- carbonitrile | 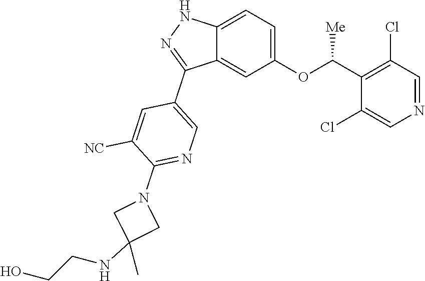 [SMILES: C[C@@H](Oc1ccc2[nH]nc(-c3cnc(N4CC(C)(NCCO)C4)c(C#N)c3)c2c1)c1c(Cl)cncc1Cl] | LCMS m/z = 538.1 (M + H); 1H NMR (400 MHz, CDCl3) δ = 13.17 (br s, 1H), 8.76 (d, J = 2.3 Hz, 1H), 8.58 (s, 2H), 8.13 (d, J = 2.3 Hz, 1H), 7.49 (d, J = 9.0 Hz, 1H), 7.16 (d, J = 2.2 Hz, 1H), 7.10 (dd, J = 2.3, 9.0 Hz, 1H), 6.14 (q, J = 6.7 Hz, 1H), 4.53 (br s, 1H), 4.15-4.11 (m, 2H), 4.07-4.04 (m, 2H), 3.47 (br t, J = 5.6 Hz, 2H), 2.62 (br d, J = 5.1 Hz, 2H), 2.34-2.29 (m, 1H), 1.76 (d, J = 6.7 Hz, 3H), 1.43 (s, 3H) |
| Example 53 (R)-5-(5-(1-(3,5- dichloropyridin-4- yl)ethoxy)-6-methoxy- 1H-indazol-3-yl)-2-(3- ((dimethylphosphoryl) methyl)-3- methylazetidin-1- yl)nicotinonitrile | 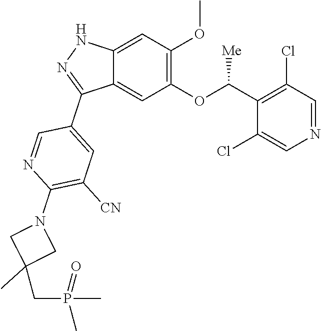 [SMILES: COc1cc2[nH]nc(-c3cnc(N4CC(C)(CP(C)(C)=O)C4)c(C#N)c3)c2cc1O[C@H](C)c1c(Cl)cncc1Cl] | LCMS m/z = 599.3 (M + H); 1H NMR (400 MHz, DMSO-d6) δ 12.96 (s, 1H), 8.71 (d, J = 2.4 Hz, 1H), 8.59 (s, 2H), 8.06 (d, J = 2.4 Hz, 1H), 7.03 (s, 1H), 7.00 (s, 1H), 5.99 (q, J = 6.8 Hz, 1H), 4.27 (d, J = 8.4 Hz, 2H), 4.03 (d, J = 8.8 Hz, 2H), 3.85 (s, 3H), 2.23 (d, J = 11.2 Hz, 2H), 1.76 (d, J = 6.8 Hz, 3H), 1.56 (s, 3H), 1.46 (s, 3H), 1.43 (s, 3H) |
| Example 54 (R)-5-(5-(1-(3,5- dichloropyridin-4- yl)ethoxy)-1H-indazol- 3-yl)-2-(3-((2-hydroxy- 2- methylpropyl)amino)- 3-methylazetidin-1- yl)nicotinonitrile | 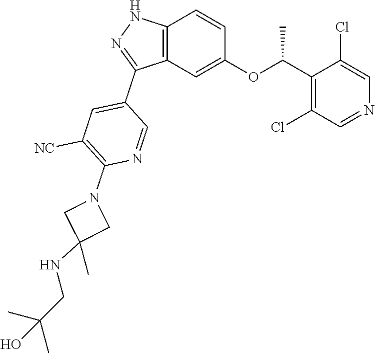 [SMILES: C[C@@H](Oc1ccc2[nH]nc(-c3cnc(N4CC(C)(NCC(C)(C)O)C4)c(C#N)c3)c2c1)c1c(Cl)cncc1Cl] | LCMS m/z = 566.3 (M + H); 1H NMR (400 MHz, DMSO-d6) δ 13.18 (s, 1H), 8.77 (s, 1H), 8.58 (s, 2H), 8.15 (s, 1H), 7.49 (d, J = 9.2 Hz, 1H), 7.16 (s, 1H), 7.11 (dd, J = 8.8 Hz, 2.0Hz, 1H), 6.14 (q, J = 6.8 Hz, 1H), 4.25-4.07 (m, 4H), 2.57- 2.52 (m, 2H), 1.76 (d, J = 6.8 Hz, 3H), 1.45 (s, 3H), 1.14 (s, 6H) |
| Example 55 (R)-5-(5-(1-(3,5- dichloropyridin-4- yl)ethoxy)-6-methoxy- 1H-indazol-3-yl)-2-(3- ((2-hydroxy-2- methylpropyl)amino)- 3-methylazetidin-1- yl)nicotinonitrile | 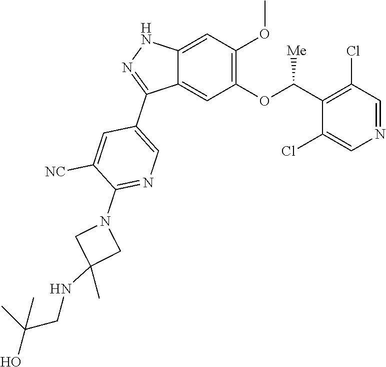 [SMILES: COc1cc2[nH]nc(-c3cnc(N4CC(C)(NCC(C)(C)O)C4)c(C#N)c3)c2cc1O[C@H](C)c1c(Cl)cncc1Cl] | LCMS m/z = 596.3 (M + H); 1H NMR (400 MHz, DMSO-d6) δ 12.97 (s, 1H), 8.71 (d, J = 2.4 Hz, 1H), 8.59 (s, 2H), 8.05 (d, J = 2.0 Hz, 1H), 7.03 (s, 1H), 7.00 (s, 1H), 6.00 (q, J = 6.8 Hz, 1H), 4.27-4.21 (m, 1H), 4.14-4.12 (m, 2H), 4.05- 4.02 (m, 2H), 3.86 (s, 3H), 2.46- 2.40 (m, 2H), 1.76 (d, J = 6.8 Hz, 3H), 1.42 (s, 3H), 1.11 (s, 6H) |
| Example 56 5-(5-((R)-1-(3,5- dichloropyridin-4- yl)ethoxy)-1H-indazol- 3-yl)-2-(3-(((R)-2- hydroxypropyl)amino)- 3-methylazetidin-1- yl)nicotinonitrile | 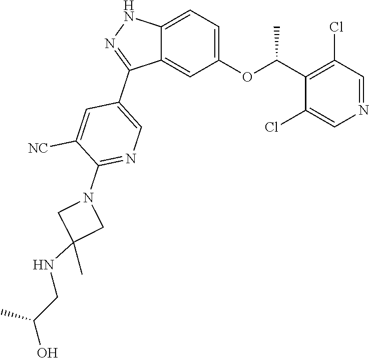 [SMILES: C[C@@H](O)CNC1(C)CN(c2ncc(-c3n[nH]c4ccc(O[C@H](C)c5c(Cl)cncc5Cl)cc34)cc2C#N)C1] | LCMS m/z = 552.2 (M + H); 1H NMR (400 MHz, DMSO-d6) δ 13.16 (s, 1H), 8.76 (d, J = 2.4 Hz, 1H), 8.57 (s, 2H), 8.13 (d, J = 2.0 Hz, 1H), 7.48 (d, J = 8.8 Hz, 1H), 7.15 (s, 1H), 7.10 (dd, J = 8.0 Hz, 2.0 Hz, 1H), 6.14 (q, J = 6.0 Hz, 1H), 4.54-4.51 (m, 1H), 4.15-4.12 (m, 2H), 4.06-4.03 (m, 2H), 3.65- 3.63 (m, 1H), 2.46-2.43 (m, 2H), 1.76 (d, J = 6.4 Hz, 3 H), 1.43 (s, 3H), 1.08 (d, J = 6.0 Hz, 3H) |
| Example 57 5-(5-((R)-1-(3,5- dichloropyridin-4- yl)ethoxy)-1H-indazol- 3-yl)-2-(3-(((S)-2- hydroxypropyl)amino)- 3-methylazetidin-1- yl)nicotinonitrile | 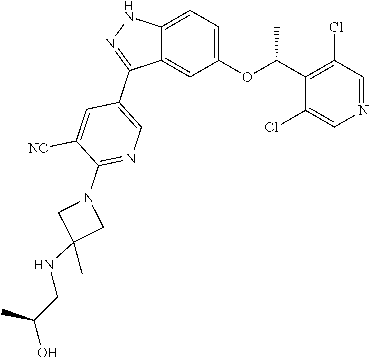 [SMILES: C[C@H](O)CNC1(C)CN(c2ncc(-c3n[nH]c4ccc(O[C@H](C)c5c(Cl)cncc5Cl)cc34)cc2C#N)C1] | LCMS m/z = 552.2 (M + H); 1H NMR (400 MHz, DMSO-d6) δ 13.17 (s, 1H), 8.76 (d, J = 2.0 Hz, 1H), 8.58 (s, 2H), 8.13 (d, J = 2.4 Hz, 1H), 7.48 (d, J = 9.2 Hz, 1H), 7.15 (s, 1H), 7.10 (dd, J = 8.8 Hz, 2.0 Hz, 1H), 6.14 (q, J = 6.4 Hz, 1H), 4.52 (d, J = 4.8 Hz, 1H), 4.13- 4.03 (m, 4H), 3.66-3.60 (m, 1H), 2.47-2.42 (m, 2H), 1.76 (d, J = 6.8 Hz, 3H), 1.43 (s, 3H), 1.08 (d, J = 6.0 Hz, 3H) |
| Example 58 3-[6-(azetidin-1-yl)-5- fluoro-3-pyridyl]-5- [(1R)-1-(3,5-dichloro- 2-methyl-4- pyridyl)ethoxy]-6- methoxy-1H-indazole | 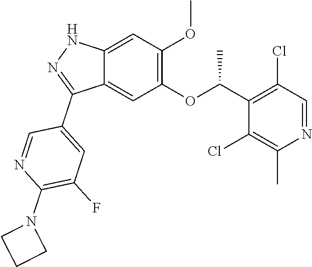 [SMILES: COc1cc2[nH]nc(-c3cnc(N4CCC4)c(F)c3)c2cc1O[C@H](C)c1c(Cl)cnc(C)c1Cl] | LCMS m/z = 502.2 (M + H); 1H NMR (400 MHz, DMSO-d6) δ = 12.88 (s, 1H), 8.43 (s, 1H), 8.18 (t, J = 1.5 Hz, 1H), 7.61 (dd, J = 1.8, 13.2 Hz, 1H), 6.99 (s, 1H), 6.95 (s, 1H), 6.01-5.95 (m, 1H), 4.14 (dt, J = 1.8, 7.5 Hz, 4H), 3.87 (s, 3H), 2.60 (s, 3H), 2.48 - 2.27 (m, 2H), 1.76 (d, J = 6.7 Hz, 3H) |
| Example 59 1-[5-[5-[(1R)-1-(3,5- dichloro-4- pyridyl)ethoxy]-6- fluoro-1H-indazol-3- yl]-2-pyridyl]-N,3- dimethyl-azetidin-3- amine | 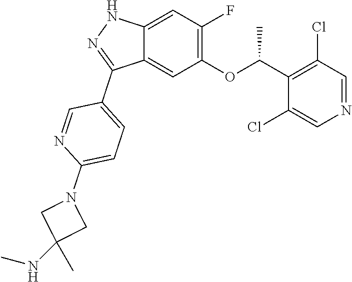 [SMILES: CNC1(C)CN(c2ccc(-c3n[nH]c4cc(F)c(O[C@H](C)c5c(Cl)cncc5Cl)cc34)cn2)C1] | LCMS m/z = 501 (M + H); 1H NMR (400 MHz, DMSO-d6) δ = 13.05 (br s, 1H), 8.63 (s, 2H), 8.47 (dd, J = 0.6, 2.3 Hz, 1H), 7.79 (dd, J = 2.4, 8.6 Hz, 1H), 7.43 (d, J = 11.0 Hz, 1H), 7.23 (d, J = 7.9 Hz, 1H), 6.52 (dd, J = 0.7, 8.6 Hz, 1H), 6.13 (q, J = 6.6 Hz, 1H), 3.83 (d, J = 7.9 Hz, 2H), 3.73 (d, J = 8.1 Hz, 2H), 2.30-2.20 (m, 4H), 1.80 (d, J = 6.6 Hz, 3H), 1.39 (s, 3H) |
| Example 60 3-[2-(5- azaspiro[2.3]hexan-5- yl)pyrimidin-5-yl]-5- [(1R)-1-(3,5-dichloro- 4-pyridyl)ethoxy]-6- fluoro-1H-indazole | 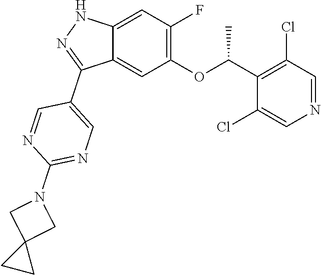 [SMILES: C[C@@H](Oc1cc2c(-c3cnc(N4CC5(CC5)C4)nc3)n[nH]c2cc1F)c1c(Cl)cncc1Cl] | LCMS m/z = 485.1 (M + H); 1H NMR (400 MHz, DMSO-d6) δ = 13.20 (br s, 1H), 8.76 (s, 2H), 8.60 (s, 2H), 7.45 (d, J = 10.9 Hz, 1H), 7.36 (d, J = 7.8 Hz, 1H), 6.17 (q, J = 6.6 Hz, 1H), 4.20 (s, 4H), 1.80 (d, J = 6.6 Hz, 3H), 0.72 (s, 4H) |
| Example 61 5-[(1R)-1-(3,5- dichloro-4- pyridyl)ethoxy]-3-[2- (3,3-dimethylazetidin- 1-yl)pyrimidin-5-yl]-6- methoxy-1H-indazole | 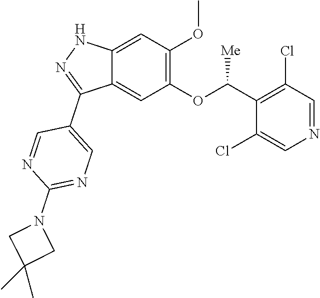 [SMILES: COc1cc2[nH]nc(-c3cnc(N4CC(C)(C)C4)nc3)c2cc1O[C@H](C)c1c(Cl)cncc1Cl] | LCMS m/z = 499.1 (M + H); 1H NMR (400 MHz, DMSO-d6) δ = 12.91 (s, 1H), 8.69 (s, 2H), 8.58 (s, 2H), 7.15 (s, 1H), 7.00 (s, 1H), 5.98 (q, J = 6.7 Hz, 1H), 3.85 (s, 3H), 3.81 (s, 4H), 1.75 (d, J = 6.7 Hz, 3H), 1.32 (s, 6H) |
| Example 62 3-[2-(5- azaspiro[2.3]hexan-5- yl)pyrimidin-5-yl]-5- [(1R)-1-(3,5-dichloro- 4-pyridyl)ethoxy]-6- methoxy-1H-indazole | 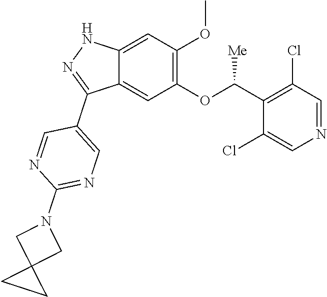 [SMILES: COc1cc2[nH]nc(-c3cnc(N4CC5(CC5)C4)nc3)c2cc1O[C@H](C)c1c(Cl)cncc1Cl] | LCMS m/z = 497.1 (M + H); 1H NMR (400 MHz, CDCl3) δ = 8.76 (s, 2H), 8.45 (s, 2H), 7.19 (s, 1H), 6.89 (s, 1H), 5.98 (q, J = 6.7 Hz, 1H), 4.30 (s, 4H), 3.92 (s, 3H), 1.83 (d, J = 6.7 Hz, 3H), 0.75 (s, 4H) |
| Example 63 (R)-N-cyclobutyl-1-(5- (5-(1-(3,5- dichloropyridin-4- yl)ethoxy)-1H-indazol- 3-yl)pyridin-2-yl)-3- methylazetidin-3-amine | 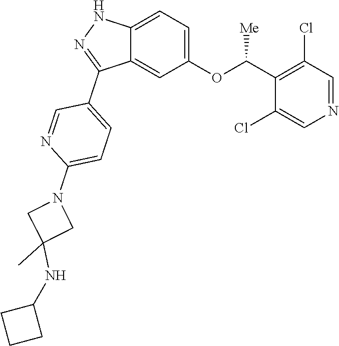 [SMILES: C[C@@H](Oc1ccc2[nH]nc(-c3ccc(N4CC(C)(NC5CCC5)C4)nc3)c2c1)c1c(Cl)cncc1Cl] | LCMS m/z = 523.3 (M + H); 1H NMR (400 MHz, DMSO-d6) δ 12.99 (s, 1H), 8.60 (s, 2H), 8.50 (s, 1H), 7.83 (d, J = 8.8 Hz, 1H), 7.45 (d, J = 9.2 Hz, 1H), 7.16 (s, 1H), 7.09 (d, J = 8.8 Hz, 1H), 6.51 (d, J = 8.4 Hz, 1H), 6.11 (q, J = 6.4 Hz, 1H), 3.80-3.72 (m, 4H), 3.32-3.30 (m, 1H), 2.14-2.08 (m, 2H), 1.76- 1.73 (m, 5H), 1.60-1.55 (m, 2H), 1.40 (s, 3H) |
| Example 64 (R)-1-(5-(5-(1-(3,5- dichloropyridin-4- yl)ethoxy)-1H-indazol- 3-yl)pyridin-2-yl)-3- methyl-N-(oxetan-3- yl)azetidin-3-amine | 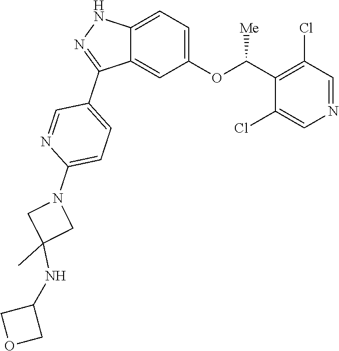 [SMILES: C[C@@H](Oc1ccc2[nH]nc(-c3ccc(N4CC(C)(NC5COC5)C4)nc3)c2c1)c1c(Cl)cncc1Cl] | LCMS m/z = 524.8 (M + H); 1H NMR (400 MHz, DMSO-d6) δ 12.98 (s, 1H), 8.59 (s, 2H), 8.49 (d, J = 1.6 Hz, 1H), 7.83 (dd, J = 8.4 Hz, 2.0 Hz, 1H), 7.45 (d, J = 8.8 Hz, 1H), 7.15 (s, 1H), 7.08 (d, J = 11.2 Hz, 1H), 6.51 (d, J = 8.4 Hz, 1H), 6.10 (q, J = 6.4 Hz, 1H), 4.65 (t, J = 6.4 Hz, 2H), 4.37 (t, J = 6.4 Hz, 2H), 4.09-4.05 (m, 1H), 3.75-3.70 (m, 4H), 1.75 (d, J = 6.8 Hz, 3H), 1.33 (s, 3H) |
| Example 65 2-(5- azaspiro[2.3]hexan-5- yl)-5-[5-[(1R)-1-(3,5- dichloro-4- pyridyl)ethoxy]-6- methoxy-1H-indazol-3- yl]pyridine-3- carbonitrile | 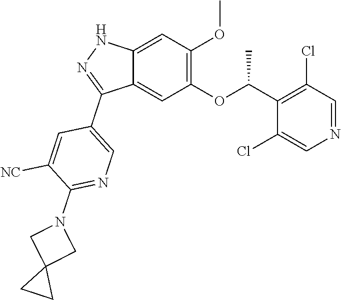 [SMILES: COc1cc2[nH]nc(-c3cnc(N4CC5(CC5)C4)c(C#N)c3)c2cc1O[C@H](C)c1c(Cl)cncc1Cl] | LCMS m/z = 521.1 (M + H); 1H NMR (400 MHz, DMSO-d6) δ = 12.96 (s, 1H), 8.73 (d, J = 2.3 Hz, 1H), 8.59 (s, 2H), 8.09 (d, J = 2.3 Hz, 1H), 7.05 (s, 1H), 7.01 (s, 1H), 6.03-5.96 (m, 1H), 5.75 (s, 1H), 4.38 (s, 4H), 3.86 (s, 3H), 1.76 (d, J = 6.6 Hz, 3H), 0.73 (s, 4H) |
| Example 66 1-(5-(5-((R)-1-(3,5- dichloropyridin-4- yl)ethoxy)-6-methoxy- 1H-indazol-3- yl)pyridin-2-yl)-3- methyl-N-((S)- tetrahydrofuran-3- yl)azetidin-3-amine | 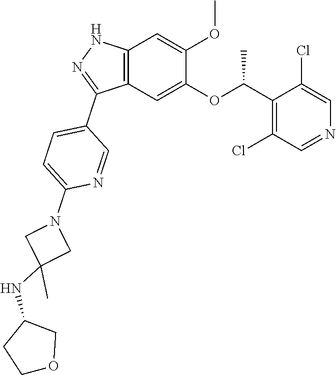 [SMILES: COc1cc2[nH]nc(-c3ccc(N4CC(C)(N[C@H]5CCOC5)C4)nc3)c2cc1O[C@H](C)c1c(Cl)cncc1Cl] | LCMS m/z = 569.3 (M + H); 1H NMR (400 MHz, DMSO-d6) δ 12.79 (s, 1H), 8.61 (s, 2H), 8.44 (d, J = 2.0 Hz, 1H), 7.77 (d, J = 6.4 Hz, 1H), 7.05 (s, 1H), 6.99 (s, 1H), 6.50 (d, J = 8.4 Hz, 1H), 5.96 (q, J = 6.8 Hz, 1H), 3.86 (s, 3H), 3.81- 3.73 (m, 6H), 3.68-3.63 (m, 1H), 3.40-3.39 (m, 1H), 3.28-3.25 (m, 1H), 2.07-1.99 (m, 1H), 1.76 (d, J = 6.4 Hz, 3H), 1.65-1.57 (m, 1H), 1.45 (s, 3H) |
| Example 67 (R)-((1-(5-(5-(1-(3,5- dichloropyridin-4- yl)ethoxy)-6-fluoro- 1H-indazol-3- yl)pyridin-2-yl)-3- methylazetidin-3- yl)methyl)dimethylphos- phine oxide | 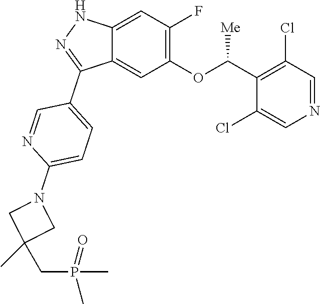 [SMILES: C[C@@H](Oc1cc2c(-c3ccc(N4CC(C)(CP(C)(C)=O)C4)nc3)n[nH]c2cc1F)c1c(Cl)cncc1Cl] | LCMS m/z = 562.2 (M + H); 1H NMR (400 MHz, DMSO-d6) δ 13.06 (s, 1H), 8.63 (s, 2H), 8.47 (d, J = 2.0 Hz, 1H), 7.79 (dd, J = 8.4 Hz, 2.4 Hz, 1H), 7.43 (d, J = 10.8 Hz, 1H), 7.23 (d, J = 7.6 Hz, 1H), 6.51 (d, J = 8.4 Hz, 1H), 6.12 (q, J = 6.8 Hz, 1H), 3.96 (d, J = 8.0 Hz, 2H), 3.76 (d, J = 7.6 Hz, 2H), 2.19 (d, J = 11.2 Hz, 2H), 1.80 (d, J = 7.2 Hz, 3H), 1.55 (s, 3H), 1.46 (s, 3H), 1.43 (s, 3H) |
| Example 68 N-cyclobutyl-1-[5-[5- [(1R)-1-(3,5-dichloro- 4-pyridyl)ethoxy]-6- fluoro-1H-indazol-3- yl]-2-pyridyl]-3- methyl-azetidin-3- amine | 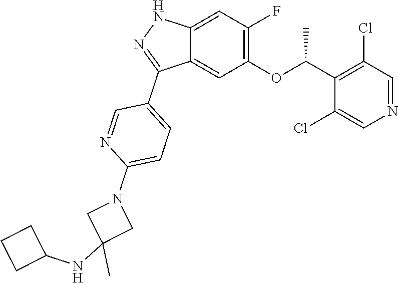 [SMILES: C[C@@H](Oc1cc2c(-c3ccc(N4CC(C)(NC5CCC5)C4)nc3)n[nH]c2cc1F)c1c(Cl)cncc1Cl] | LCMS m/z = 541 (M + H); 1H NMR (400 MHz, DMSO-d6) δ = 13.04 (br s, 1H), 8.63 (s, 2H), 8.47 (d, J = 2.0 Hz, 1H), 7.78 (dd, J = 2.4, 8.6 Hz, 1H), 7.43 (d, J = 11.0 Hz, 1H), 7.23 (d, J = 7.8 Hz, 1H), 6.51 (d, J = 8.3 Hz, 1H), 6.13 (q, J = 6.8 Hz, 1H), 3.81-3.71 (m, 4H), 2.15- 2.06 (m, 2H), 1.80 (d, J = 6.8 Hz, 3H), 1.78-1.70 (m, 2H), 1.66- 1.47 (m, 3H) |
| Example 69 (R)-N-((1-(5-(5-(1- (3,5-dichloropyridin-4- yl)ethoxy)-1H-indazol- 3-yl)pyridin-2-yl)-3- methylazetidin-3- yl)methyl)methane- sulfonamide | 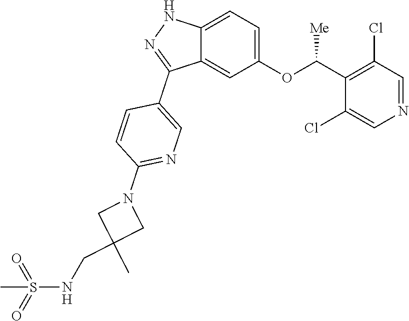 [SMILES: C[C@@H](Oc1ccc2[nH]nc(-c3ccc(N4CC(C)(CNS(C)(=O)=O)C4)nc3)c2c1)c1c(Cl)cncc1Cl] | LCMS m/z = 561.2 (M + H); 1H NMR (400 MHz, DMSO-d6) δ 12.99 (s, 1H), 8.60 (s, 2H), 8.50 (d, J = 2.0 Hz, 1H), 7.85 (dd, J = 8.8 Hz, 2.0 Hz, 1H), 7.46 (d, J = 9.2 Hz, 1H), 7.29 (t, J = 6.8 Hz, 1H), 7.17 (d, J = 1.6 Hz, 1H), 7.09 (dd, J = 8.8 Hz, 2.4 Hz, 1H), 6.51 (d, J = 8.8 Hz, 1H), 6.11 (q, J = 6.8 Hz, 1H), 3.89 (d, J = 8.4 Hz, 2H), 3.63 (d, J = 8.0 Hz, 2H), 3.15 (d, J = 6.8 Hz, 2H), 2.94 (s, 3H), 1.76 (d, J = 6.4 Hz, 3H), 1.32 (s, 3H) |
| Example 70 1-[5-[5-[(1R)-1-(3,5- dichloro-2-methyl-4- pyridyl)ethoxy]-1H- indazol-3-yl]-2- pyridyl]-3-methyl-N- (oxetan-3-yl)azetidin- 3-amine | 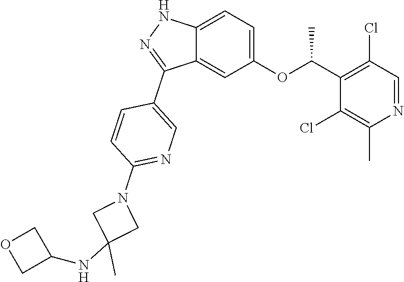 [SMILES: Cc1ncc(Cl)c([C@@H](C)Oc2ccc3[nH]nc(-c4ccc(N5CC(C)(NC6COC6)C5)nc4)c3c2)c1Cl] | LCMS m/z = 539.2 (M + H); 1H NMR (400 MHz, DMSO-d6) δ = 13.02 (br s, 1H), 8.44 (s, 1H), 8.42 (d, J = 1.8 Hz, 1H), 7.89 (dd, J = 1.9, 8.6 Hz, 1H), 7.46 (d, J = 9.0 Hz, 1H), 7.11-7.05 (m, 2H), 6.57 (br d, J = 8.3 Hz, 1H), 6.09 (q, J = 6.6 Hz, 1H), 4.78-4.24 (m, 5H), 4.20-3.76 (m, 4H), 2.58 (s, 3H), 1.76 (d, J = 6.7 Hz, 3H), 1.45 (br s, 3H) |
| Example 71 N-cyclobutyl-1-[5-[5- [(1R)-1-(3,5-dichloro- 2-methyl-4- pyridyl)ethoxy]-1H- indazol-3-yl]-2- pyridyl]-3-methyl- azetidin-3-amine | 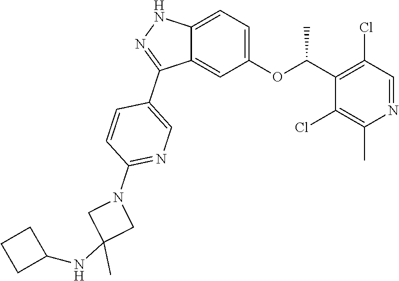 [SMILES: Cc1ncc(Cl)c([C@@H](C)Oc2ccc3[nH]nc(-c4ccc(N5CC(C)(NC6CCC6)C5)nc4)c3c2)c1Cl] | LCMS m/z = 537.2 (M + H); 1H NMR (400 MHz, DMSO-d6) δ = 13.00 (s, 1H), 8.44 (s, 1H), 8.41 (d, J = 2.0 Hz, 1H), 7.86 (dd, J = 2.3, 8.6 Hz, 1H), 7.46 (d, J = 9.0 Hz, 1H), 7.11-7.04 (m, 2H), 6.53 (d, J = 8.6 Hz, 1H), 6.12-6.06 (m, 1H), 3.95-3.84 (m, 2H), 3.79 (d, J = 7.9 Hz, 2H), 3.40-3.16 (m, 2H), 2.58 (s, 3H), 2.20-2.07 (m, 2H), 1.90 (br d, J = 7.2 Hz, 2H), 1.76 (d, J = 6.6 Hz, 3H), 1.69-1.50 (m, 2H), 1.46 (s, 3H) |
| Example 72 1-[5-[5-[(1R)-1-(3,5- dichloro-2-methyl-4- pyridyl)ethoxy]-1H- indazol-3-yl]-2- pyridyl]-N,N-diethyl-3- methyl-azetidin-3- amine | 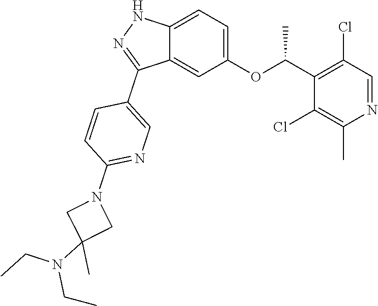 [SMILES: CCN(CC)C1(C)CN(c2ccc(-c3n[nH]c4ccc(O[C@H](C)c5c(Cl)cnc(C)c5Cl)cc34)cn2)C1] | LCMS m/z = 540.2 (M + H); 1H NMR (400 MHz, DMSO-d6) δ = 13.07 (br s, 1H), 10.26 (br s, 1H), 8.50-8.39 (m, 2H), 7.95 (br d, J = 6.7 Hz, 1H), 7.46 (br d, J = 8.6 Hz, 1H), 7.13-7.05 (m, 2H), 6.65 (br d, J = 9.0 Hz, 1H), 6.10 (br d, J = 6.6 Hz, 1H), 4.50-3.92 (m, 2H), 3.82- 3.70 (m, 2H), 3.30-3.03 (m, 2H), 2.58 (s, 3H), 2.48-2.32 (m, 3H), 1.79-1.71 (m, 4H), 1.32 (br t, J = 6.8 Hz, 2H), 1.03 (br t, J = 7.0 Hz, 4H) |
| Example 73 1-(5-(5-((R)-1-(3,5- dichloropyridin-4- yl)ethoxy)-6-methoxy- 1H-indazol-3- yl)pyridin-2-yl)-3- methyl-N-((R)- tetrahydrofuran-3- yl)azetidin-3-amine | 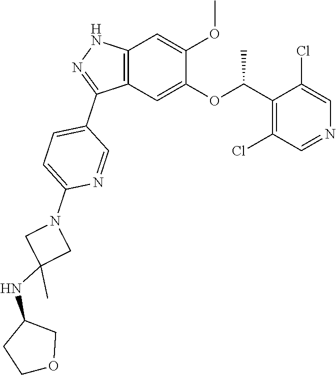 [SMILES: COc1cc2[nH]nc(-c3ccc(N4CC(C)(N[C@@H]5CCOC5)C4)nc3)c2cc1O[C@H](C)c1c(Cl)cncc1Cl] | LCMS m/z = 569.2 (M + H); 1H NMR (400 MHz, DMSO-d6) δ 12.79 (s, 1H), 8.61 (s, 2H), 8.44 (d, J = 1.6 Hz, 1H), 7.77 (dd, J = 8.4 Hz, 2.0 Hz, 1H), 7.05 (s, 1H), 6.99 (s, 1H), 6.51 (d, J = 8.8 Hz, 1H), 5.96 (q, J = 6.8 Hz, 1H), 3.86 (s, 3H), 3.81-3.74 (m, 6H), 3.68-3.63 (m, 1H), 3.45-3.40 (m, 1H), 3.32- 3.20 (m, 1H), 2.08-2.00 (m, 1H), 1.75 (d, J = 6.8 Hz, 3H), 1.66-1.58 (m, 1H), 1.45 (s, 3H) |
| Example 74 (R)-N-((1-(5-(5-(1- (3,5-dichloropyridin-4- yl)ethoxy)-1H-indazol- 3-yl)pyridin-2-yl)-3- methylazetidin-3- yl)methyl)-N- methylmethanesulfona- mide | 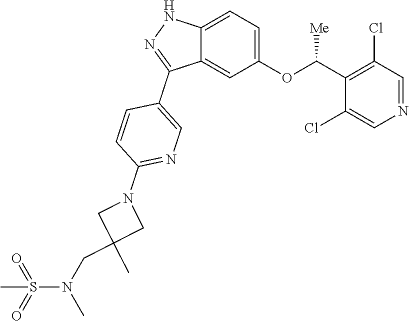 [SMILES: C[C@@H](Oc1ccc2[nH]nc(-c3ccc(N4CC(C)(CN(C)S(C)(=O)=O)C4)nc3)c2c1)c1c(Cl)cncc1Cl] | LCMS m/z = 575.2 (M + H); 1H NMR (400 MHz, DMSO-d6) δ 12.99 (s, 1H), 8.60 (s, 2H), 8.51 (d, J = 2.0 Hz, 1H), 7.85 (dd, J = 8.4 Hz, 2.0 Hz, 1H), 7.46 (d, J = 8.8 Hz, 1H), 7.17 (d, J = 1.6 Hz, 1H), 7.09 (dd, J = 9.2 Hz, 2.4 Hz, 1H), 6.53 (d, J = 8.4 Hz, 1H), 6.11 (q, J = 6.4 Hz, 1H), 3.93 (d, J = 8.0 Hz, 2H), 3.70 (d, J = 8.0 Hz, 2H), 3.30 (s, 2H), 2.91 (s, 3H), 2.83 (s, 3H), 1.76 (d, J = 6.8 Hz, 3H), 1.39 (s, 3H) |
| Example 75 3-[6-[3-(cyclobutoxy)- 3-methyl-azetidin-1- yl]-3-pyridy1]-5-[(1R)- 1-(3,5-dichloro-2- methyl-4- pyridyl)ethoxy]-1H- indazole | 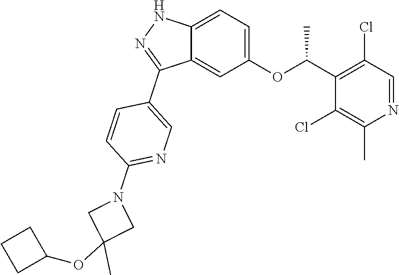 | LCMS m/z = 551.2 (M + H); 1H NMR (400 MHz, DMSO-d6) δ = 12.98 (s, 1H), 8.43 (s, 1H), 8.41 (d, J = 1.7 Hz, 1H), 7.86 (dd, J = 2.4, 8.6 Hz, 1H), 7.45 (d, J = 9.5 Hz, 1H), 7.11-7.05 (m, 2H), 6.53 (d, J = 8.8 Hz, 1H), 6.09 (q, J = 6.8 Hz, 1H), 4.12-4.00 (m, 1H), 3.87 (q, J = 8.4 Hz, 4H), 2.57 (s, 3H), 2.24- 2.15 (m, 2H), 2.00-1.89 (m, 2H), 1.76 (d, J = 6.8 Hz, 3H), 1.65-1.41 (m, 5H) |
| Example 76 1-[5-[5-[(1R)-1-(3,5- dichloro-2-methyl-4- pyridyl)ethoxy]-1H- indazol-3-yl]-2- pyridyl]-N,3-dimethyl- azetidin-3-amine | 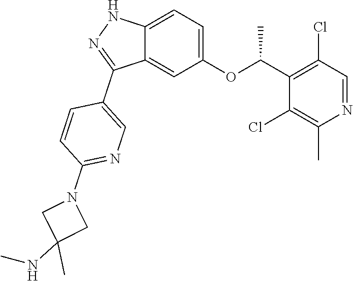 [SMILES: CNC1(C)CN(c2ccc(-c3n[nH]c4ccc(O[C@H](C)c5c(Cl)cnc(C)c5Cl)cc34)cn2)C1] | LCMS m/z = 497.1 (M + H); 1H NMR (400 MHz, DMSO-d6) δ = 12.98 (br s, 1H), 8.44 (s, 1H), 8.39 (d, J = 1.8 Hz, 1H), 7.85 (d, J = 2.3 Hz, 1H), 7.45 (d, J = 9.0 Hz, 1H), 7.11-7.04 (m, 2H), 6.50 (d, J = 8.7 Hz, 1H), 6.09 (q, J = 6.7 Hz, 1H), 3.82 (d, J = 7.9 Hz, 2H), 3.73 (d, J = 7.9 Hz, 2H), 2.58 (s, 3H), 2.24 (s, 4H), 1.75 (d, J = 6.6 Hz, 3H), 1.41- 1.37 (m, 3H) |
| Example 77 4-[1-[5-[5-[(1R)-1-(3,5- dichloro-2-methyl-4- pyridyl)ethoxy]-1H- indazol-3-yl]-2- pyridyl]-3-methyl- azetidin-3- yl]morpholine | 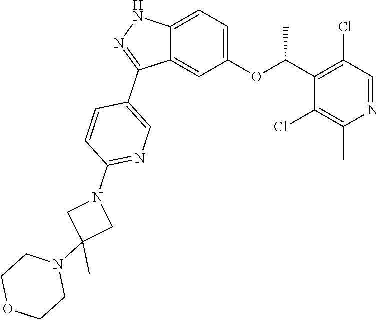 [SMILES: Cc1ncc(Cl)c([C@@H](C)Oc2ccc3[nH]nc(-c4ccc(N5CC(C)(N6CCOCC6)C5)nc4)c3c2)c1Cl] | LCMS m/z = 553.1 (M + H); 1H NMR (400 MHz, DMSO-d6) δ = 12.98 (s, 1H), 8.44 (s, 1H), 8.41 (d, J = 1.8 Hz, 1H), 7.85 (dd, J = 2.3, 8.7 Hz, 1H), 7.45 (d, J = 9.0 Hz, 1H), 7.11-7.04 (m, 2H), 6.51 (d, J = 8.7 Hz, 1H), 6.09 (q, J = 6.6 Hz, 1H), 3.82 (d, J = 7.9 Hz, 2H), 3.69 (d, J = 7.8 Hz, 2H), 3.64-3.56 (m, 4H), 2.57 (s, 3H), 2.45-2.39 (m, 4H), 1.75 (d, J = 6.7 Hz, 3H), 1.37- 1.32 (m, 3H) |
| Example 78 1-[5-[5-[(1R)-1-(3,5- dichloro-2-methyl-4- pyridyl)ethoxy]-1H- indazol-3-yl]-2- pyridyl]-N,N,3- trimethyl-azetidin-3- amine | 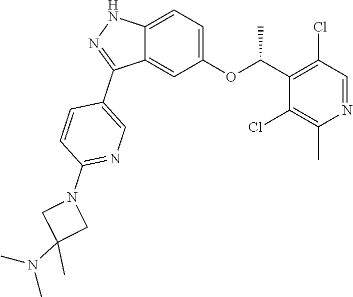 [SMILES: Cc1ncc(Cl)c([C@@H](C)Oc2ccc3[nH]nc(-c4ccc(N5CC(C)(N(C)C)C5)nc4)c3c2)c1Cl] | LCMS m/z = 511.1 (M + H); 1H NMR (400 MHz, DMSO-d6) δ = 12.98 (s, 1H), 8.44 (s, 1H), 8.40 (d, J = 1.8 Hz, 1H), 7.85 (dd, J = 2.3, 8.7 Hz, 1H), 7.45 (d, J = 9.0 Hz, 1H), 7.11-7.05 (m, 2H), 6.51 (d, J = 8.7 Hz, 1H), 6.09 (q, J = 6.6 Hz, 1H), 3.78 (d, J = 7.8 Hz, 2H), 3.69 (d, J = 7.7 Hz, 2H), 2.58 (s, 3H), 2.13 (s, 6H), 1.76 (d, J = 6.6 Hz, 3H), 1.31 (s, 3H) |
| Example 79 (R)-1-(5-(5-(1-(3,5- dichloropyridin-4- yl)ethoxy)-1H-indazol- 3-yl)pyrimidin-2-yl)-N- (2-methoxyethyl)-N,3- dimethylazetidin-3- amine | 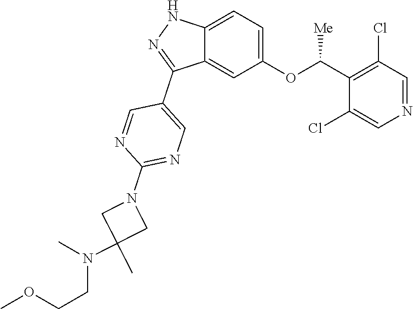 [SMILES: COCCN(C)C1(C)CN(c2ncc(-c3n[nH]c4ccc(O[C@H](C)c5c(Cl)cncc5Cl)cc34)cn2)C1] | LCMS m/z = 542.3 (M + H); 1H NMR (400 MHz, DMSO-d6) δ 13.13 (s, 1H), 8.76 (s, 2H), 8.57 (s, 2H), 7.47 (d, J = 8.8 Hz, 1H), 7.25 (d, J = 2.0 Hz, 1H), 7.11 (dd, J = 8.8 Hz, 2.4 Hz, 1H), 6.15 (q, J = 6.8 Hz, 1H), 3.86 (d, J = 8.8 Hz, 2H), 3.78 (d, J = 8.4 Hz, 2H), 3.39 (t, J = 5.6 Hz, 2H), 3.25 (s, 3H), 2.44 (t, J = 6.0 Hz, 2H), 2.16 (s, 3H), 1.75 (d, J = 6.8 Hz, 3H), 1.30 (s, 3H) |
| Example 80 (R)-4-(1-(5-(5-(1-(3,5- dichloropyridin-4- yl)ethoxy)-1H-indazol- 3-yl)-3-fluoropyridin- 2-yl)-3-methylazetidin- 3-yl)morpholine | 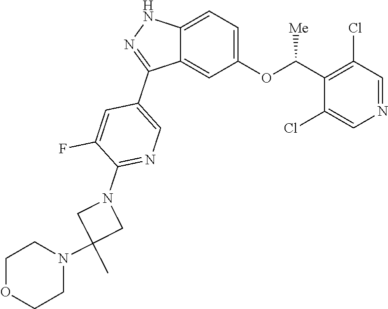 [SMILES: C[C@@H](Oc1ccc2[nH]nc(-c3cnc(N4CC(C)(N5CCOCC5)C4)c(F)c3)c2c1)c1c(Cl)cncc1Cl] | LCMS m/z = 557.3 (M + H); 1H NMR (400 MHz, DMSO-d6) δ 13.12 (s, 1H), 8.57 (s, 2H), 8.36 (s, 1H), 7.71 (dd, J = 13.2 Hz, 1.2 Hz, 1H), 7.47 (d, J = 8.8 Hz, 1H), 7.19 (d, J = 1.6 Hz, 1H), 7.10 (dd, J = 8.8 Hz, 2.0 Hz, 1H), 6.14 (q, J = 6.4 Hz, 1H), 3.98 (d, J = 7.6 Hz, 2H), 3.81 (dd, J = 8.0 Hz, 1.6 Hz, 2H), 3.61-3.59 (m, 4H), 2.43-2.41 (m, 4H), 1.76 (d, J = 6.8 Hz, 3H), 1.35 (s, 3H) |
| Example 81 (R)-1-(5-(5-(1-(3,5- dichloropyridin-4- yl)ethoxy)-1H-indazol- 3-yl)-3-fluoropyridin- 2-yl)-N,N,3- trimethylazetidin-3- amine | 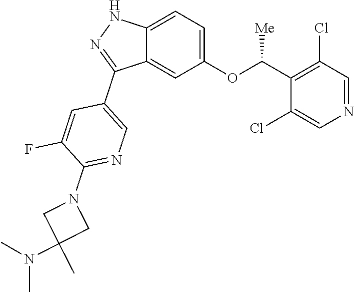 | LCMS m/z = 440.3 (M + H); 1H NMR (400 MHz, DMSO-d6) δ 13.12 (s, 1H), 8.57 (s, 2H), 8.36 (t, J = 1.6 Hz, 1H), 7.70 (dd, J = 13.2 Hz, 2.0 Hz, 1H), 7.47 (d, J = 8.8 Hz, 1H), 7.20 (d, J = 2.0 Hz, 1H), 7.10 (dd, J = 9.2 Hz, 2.4 Hz, 1H), 6.14 (q, J = 6.8 Hz, 1H), 3.93 (d, J = 8.0 Hz, 2H), 3.80 (dd, J = 8.4 Hz, 2.0 Hz, 2H), 2.11 (s, 6H), 1.76 (d, J = 6.8 Hz, 3H), 1.32 (s, 3H) |
| Example 82 (R)-1-(5-(5-(1-(3,5- dichloropyridin-4- yl)ethoxy)-6-methoxy- 1H-indazol-3- yl)pyrimidin-2-yl)-N- isopropyl-3- methylazetidin-3-amine | 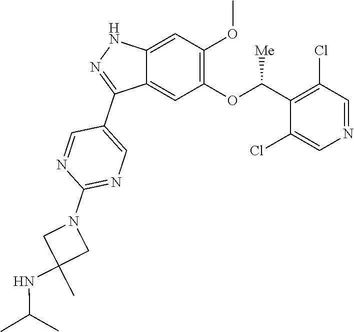 [SMILES: COc1cc2[nH]nc(-c3cnc(N4CC(C)(NC(C)C)C4)nc3)c2cc1O[C@H](C)c1c(Cl)cncc1Cl] | LCMS m/z = 542.2 (M + H); 1H NMR (400 MHz, DMSO-d6) δ 12.92 (s, 1H), 8.69 (s, 2H), 8.59 (s, 2H), 7.14 (s, 1H), 7.00 (s, 1H), 5.98 (q, J = 6.8 Hz, 1H), 3.87 (s 4H), 3.85 (s, 3H), 2.97-2.94 (m, 1H), 1.75 (d, J = 6.8 Hz, 3H), 1.44 (s, 3H), 1.00 (d, J = 6.0 Hz, 6H) |
| Example 83 (R)-2-((1-(5-(5-(1-(3,5- dichloropyridin-4- yl)ethoxy)-6-methoxy- 1H-indazol-3- yl)pyridin-2-yl)-3- methylazetidin-3- yl)amino)ethan-1-ol | 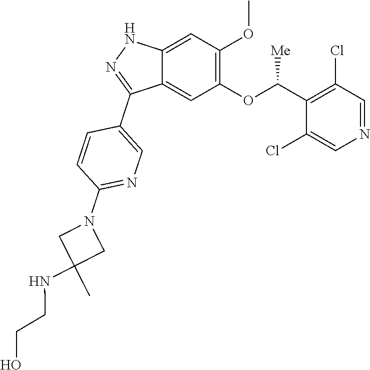 [SMILES: COc1cc2[nH]nc(-c3ccc(N4CC(C)(NCCO)C4)nc3)c2cc1O[C@H](C)c1c(Cl)cncc1Cl] | LCMS m/z = 543 (M + H); 1H NMR (400 MHz, DMSO-d6) δ12.80 (s, 1H), 8.61 (s, 2H), 8.44 (d, J = 1.6 Hz, 1H), 7.79 (dd, J = 8.4 Hz, 1.6 Hz, 1H), 7.05 (s, 1H), 6.99 (s, 1H), 6.52 (d, J = 8.8 Hz, 1H), 5.99 (q, J = 6.4 Hz, 1H), 4.56-4.4 (m, 1H), 3.86 (s, 3H), 3.84-3.75 (m, 4H), 3.47-3.46 (m, 2H), 2.62 (t, J = 5.6 Hz, 2H), 1.76 (d, J = 6.8 Hz, 3H), 1.42 (s, 3H) |
| Example 84 (R)-N-((1-(5-(5-(1- (3,5-dichloropyridin-4- yl)ethoxy)-6-methoxy- 1H-indazol-3- yl)pyrimidin-2-yl)-3- methylazetidin-3- yl)methyl)propan-2- amine | 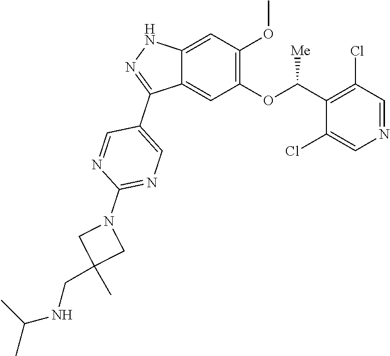 [SMILES: COc1cc2[nH]nc(-c3cnc(N4CC(C)(CNC(C)C)C4)nc3)c2cc1O[C@H](C)c1c(Cl)cncc1Cl] | LCMS m/z = 556.3 (M + H); 1H NMR (400 MHz, DMSO-d6) δ 12.93 (s, 1H), 8.68 (s, 2H), 8.58 (s, 2H), 7.13 (s, 1H), 7.00 (s, 1H), 5.97 (q, J = 6.0 Hz, 1H), 3.91 (d, J = 8.8 Hz, 2H), 3.85 (s, 3H), 3.68 (d, J = 8.4 Hz, 2H), 2.72-2.67 (m, 1H), 2.64 (s, 2H), 1.75 (d, J = 6.8 Hz, 3H), 1.29 (s, 3H), 1.00 (d, J = 6.4 Hz, 6H) |
| Example 85 (R)-1-(5-(5-(1-(3,5- dichloropyridin-4- yl)ethoxy)-1H-indazol- 3-yl)-3-fluoropyridin- 2-yl)-N-(2- methoxyethyl)-N,3- dimethylazetidin-3- amine | 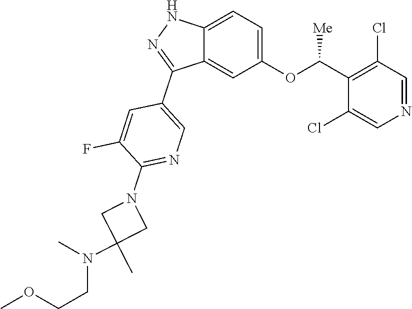 [SMILES: COCCN(C)C1(C)CN(c2ncc(-c3n[nH]c4ccc(O[C@H](C)c5c(Cl)cncc5Cl)cc34)cc2F)C1] | LCMS m/z = 559.3 (M + H); 1H NMR (400 MHz, DMSO-d6) δ 13.12 (s, 1H), 8.57 (s, 2H), 8.36 (s, 1H), 7.73 (dd, J = 13.2 Hz, 1.6 Hz, 1H), 7.48 (d, J = 9.2 Hz, 1H), 7.20 (d, J = 2.0 Hz, 1H), 7.11 (dd, J = 9.2 Hz, 2.4 Hz, 1H), 6.17 (q, J = 6.8 Hz, 1H), 3.90 (d, J = 7.6 Hz, 2H), 3.83 (d, J = 8.0 Hz, 2H), 3.39 (t, J = 6.0 Hz, 2H), 3.24 (s, 3H), 2.43 (t, J = 6.0 Hz, 2H), 2.14 (s, 3H), 1.77 (d, J = 6.4 Hz, 3H), 1.33 (s, 3H) |
| Example 86 (R)-2-((1-(5-(5-(1-(3,5- dichloropyridin-4- yl)ethoxy)-6-methoxy- 1H-indazol-3- yl)pyrimidin-2-yl)-3- methylazetidin-3- yl)amino)ethan-1-ol | 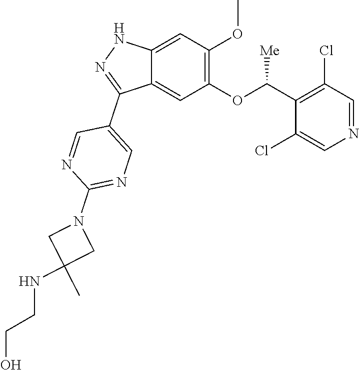 [SMILES: COc1cc2[nH]nc(-c3cnc(N4CC(C)(NCCO)C4)nc3)c2cc1O[C@H](C)c1c(Cl)cncc1Cl] | LCMS m/z = 543.8 (M + H); 1H NMR (400 MHz, DMSO-d6) δ 12.93 (s, 1H), 8.69 (s, 2H), 8.58 (s, 2H), 7.14 (s, 1H), 7.00 (s, 1H), 5.98 (q, J = 6.8 Hz, 1H), 4.52 (t, J = 5.2 Hz, 1H), 3.93 (d, J = 8.8 Hz, 2H), 3.85 (s, 3H), 3.83 (d, J = 8.4 Hz, 2H), 3.49-3.45 (m, 2H), 2.61 (t, J = 5.6 Hz, 2H), 1.75 (d, J = 6.8 Hz, 3H), 1.41 (s, 3H) |
| Example 87 (R)-N-(1-(3-cyano-5- (5-(1-(3,5- dichloropyridin-4- yl)ethoxy)-1H-indazol- 3-yl)pyridin-2-yl)-3- methylazetidin-3-yl)-2- (dimethylamino)acetamide | 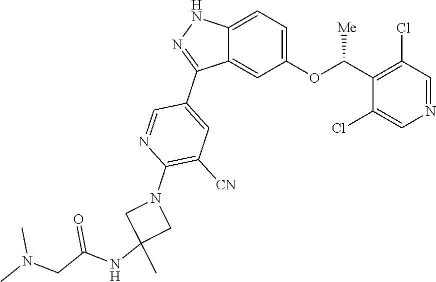 [SMILES: C[C@@H](Oc1ccc2[nH]nc(-c3cnc(N4CC(C)(NC(=O)CN(C)C)C4)c(C#N)c3)c2c1)c1c(Cl)cncc1Cl] | LCMS m/z = 579.5 (M + H); 1H NMR (400 MHz, DMSO-d6) δ 13.18 (s, 1H), 8.77 (d, J = 2.4 Hz, 1H), 8.58 (s, 2H), 8.36 (s, 1H), 8.15 (d, J = 2.4 Hz, 1H), 7.49 (d, J = 9.2 Hz, 1H), 7.16 (d, J = 1.6 Hz,, 1H), 7.11 (dd, J = 8.8 Hz, 2.0 Hz, 1H), 6.14 (q, J = 6.8 Hz, 1H), 4.42 (d, J = 9.6 Hz, 2H), 4.18 (d, J = 9.2 Hz, 2H), 2.91 (s, 2H), 2.24 (s, 6H), 1.76 (d, J = 6.4 Hz, 3H), 1.58 (s, 3H) |
| Example 88 (R)-5-(5-(1-(3,5- dichloropyridin-4- yl)ethoxy)-1H-indazol- 3-yl)-2-(3-((2- methoxyethyl)amino)- 3-methylazetidin-1- yl)nicotinonitrile | 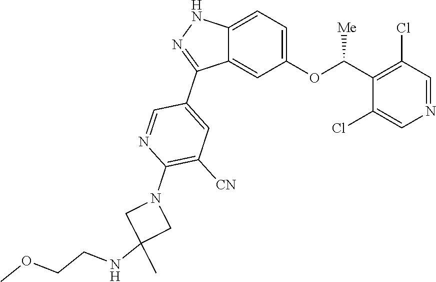 [SMILES: COCCNC1(C)CN(c2ncc(-c3n[nH]c4ccc(O[C@H](C)c5c(Cl)cncc5Cl)cc34)cc2C#N)C1] | LCMS m/z = 552.2 (M + H); 1H NMR (400 MHz, DMSO-d6) δ 13.18 (s, 1H), 8.76 (d, J = 2.0 Hz, 1H), 8.60-8.56 (m, 2H), 8.13 (d, J = 2.0 Hz, 1H), 7.49 (d, J = 8.8 Hz, 1H), 7.15 (d, J = 1.6 Hz, 1H), 7.10 (dd, J = 11.2 Hz, 2.4 Hz, 1H), 6.14 (q, J = 6.8 Hz, 1H), 4.14-4.11 (m, 2H), 4.06-4.03 (m, 2H), 3.41 (t, J = 5.6 Hz, 2H), 3.27 (s, 3H), 2.72 (t, J = 5.6 Hz, 2H), 1.76 (d, J = 6.4 Hz, 3H), 1.44 (s, 3H) |
| Example 89 (R)-2-((1-(5-(5-(1-(3,5- dichloropyridin-4- yl)ethoxy)-1H-indazol- 3-yl)pyridin-2-yl)-3- methylazetidin-3- yl)amino)ethan-1-ol | 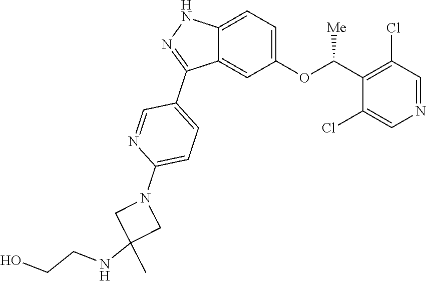 [SMILES: C[C@@H](Oc1ccc2[nH]nc(-c3ccc(N4CC(C)(NCCO)C4)nc3)c2c1)c1c(Cl)cncc1Cl] | LCMS m/z = 513.3 (M + H); 1H NMR (400 MHz, DMSO-d6) δ 12.99 (s, 1H), 8.59 (d, J = 1.2 Hz, 2H), 8.50 (s, 1H), 7.83 (d, J = 8.8 Hz, 1H), 7.45 (d, J = 8.8 Hz, 1H), 7.16 (s, 1H), 7.08 (d, J = 8.8 Hz, 1H), 6.52 (d, J = 8.8 Hz, 1H), 6.11 (q, J = 6.4 Hz, 1H), 4.56 (brs, 1H), 3.84-3.82 (m, 2H), 3.77-3.75 (m, 2H), 3.48-3.45 (m, 2H), 2.62 (t, J = 5.6 Hz, 2H), 1.76 (d, J = 6.8 Hz, 3H), 1.42 (s, 3H) |
| Example 90 (S)-1-((1-(5-(5-((R)-1- (3,5-dichloropyridin-4- yl)ethoxy)-1H-indazol- 3-yl)pyridin-2-yl)-3- methylazetidin-3- yl)amino)propan-2-ol | 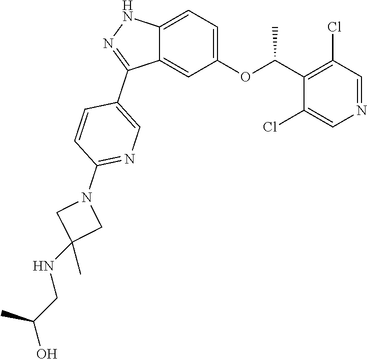 [SMILES: C[C@H](O)CNC1(C)CN(c2ccc(-c3n[nH]c4ccc(O[C@H](C)c5c(Cl)cncc5Cl)cc34)cn2)C1] | LCMS m/z = 527.3 (M + H); 1H NMR (400 MHz, DMSO-d6) δ 12.97 (s, 1H), 8.59 (s, 2H), 8.50 (d, J = 2.0 Hz, 1H), 7.84 (dd, J = 8.8 Hz, 2.4 Hz, 1H), 7.45 (d, J = 9.2 Hz, 1H), 7.16 (s, 1H), 7.09 (dd, J = 8.8 Hz, 2.0 Hz, 1H), 6.52 (d, J = 8.4 Hz, 1H), 6.11 (q, J = 6.4 Hz, 1H), 4.51 (s, 1H), 3.85-3.75 (m, 4H), 3.66-3.61 (m, 1H), 2.47-2.46 (m, 2H), 1.76 (d, J = 6.4 Hz, 3H), 1.42 (s, 3H), 1.08 (d, J = 6.0 Hz, 3H) |
| Example 91 (R)-1-((1-(5-(5-((R)-1- (3,5-dichloropyridin-4- yl)ethoxy)-1H-indazol- 3-yl)pyridin-2-yl)-3- methylazetidin-3- yl)amino)propan-2-ol | 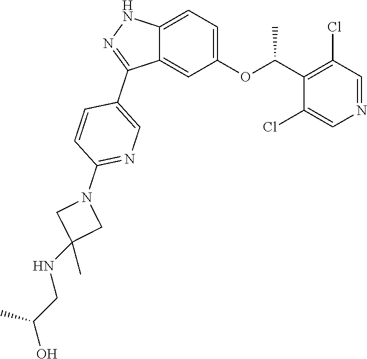 [SMILES: C[C@@H](O)CNC1(C)CN(c2ccc(-c3n[nH]c4ccc(O[C@H](C)c5c(Cl)cncc5Cl)cc34)cn2)C1] | LCMS m/z = 527.3 (M + H); 1H NMR (400 MHz, DMSO-d6) δ 12.99 (s, 1H), 8.60 (s, 2H), 8.50 (d, J = 2.0 Hz, 1 H), 7.83 (dd, J = 8.8 Hz, 2.4 Hz, 1H), 7.45 (d, J = 8.8 Hz, 1H), 7.16 (d, J = 2.0 Hz, 1H), 7.09 (dd, J = 8.8 Hz, 2.0 Hz, 1H), 6.52 (d, J = 8.4 Hz, 1 H), 6.11 (q, J = 6.8 Hz, 1H), 4.52 (d, J = 4.4 Hz 1H), 3.84-3.75 (m, 4 H), 3.66-3.61 (m, 1H), 2.47-2.42 (m, 2H), 1.75 (d, J = 6.4 Hz, 3 H), 1.42 (s, 3H), 1.08 (d, J = 6.4 Hz, 3H) |
| Example 92 (R)-2-((1-(5-(5-(1-(3,5- dichloropyridin-4- yl)ethoxy)-1H-indazol- 3-yl)pyridin-2-yl)-3- methylazetidin-3- yl)oxy)ethan-1-ol | 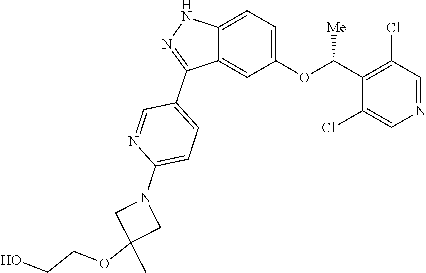 [SMILES: C[C@@H](Oc1ccc2[nH]nc(-c3ccc(N4CC(C)(OCCO)C4)nc3)c2c1)c1c(Cl)cncc1Cl] | LCMS m/z = 514.2 (M + H); 1H NMR (400 MHz, DMSO-d6) δ 13.00 (s, 1H), 8.60 (s, 2H), 8.51 (d, J = 2.0 Hz, 1H), 7.86 (dd, J = 8.8 Hz, 2.0 Hz, 1H), 7.46 (d, J = 8.8 Hz, 1H), 7.17 (d, J = 1.6 Hz, 1H), 7.09 (dd, J = 8.8 Hz, 2.0 Hz, 1H), 6.56 (d, J = 8.8 Hz, 1H), 6.11 (q, J = 6.4 Hz, 1H), 4.66 (brs, 1H), 3.96 (d, J = 8.8 Hz, 2H), 3.86 (d, J = 8.4 Hz, 2H), 3.52-3.51 (m, 2H), 3.44 (t, J = 5.2 Hz, 2H), 1.76 (d, J = 6.8 Hz, 3H), 1.52 (s, 3H) |
| Example 93 (R)-1-((1-(5-(5-(1-(3,5- dichloropyridin-4- yl)ethoxy)-1H-indazol- 3-yl)pyridin-2-yl)-3- methylazetidin-3- yl)oxy)-2- methylpropan-2-ol | 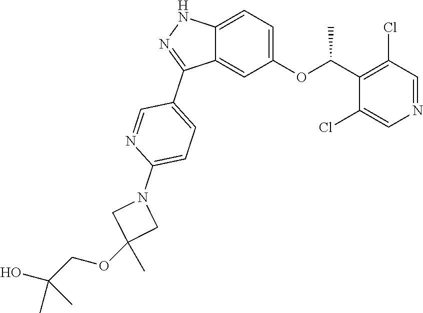 [SMILES: C[C@@H](Oc1ccc2[nH]nc(-c3ccc(N4CC(C)(OCC(C)(C)O)C4)nc3)c2c1)c1c(Cl)cncc1Cl] | LCMS m/z = 542.3 (M + H); 1H NMR (400 MHz, DMSO-d6) δ 13.01 (s, 1H), 8.60 (s, 2H), 8.51 (s, 1H), 7.87-7.85 (m, 1H), 7.46 (d, J = 9.2 Hz, 1H), 7.17 (s, 1H), 7.09 (d, J = 9.2 Hz, 1H), 6.57 (d, J = 8.4 Hz, 1H), 6.12 (q, J = 6.4 Hz, 1H), 4.38 (s, 1H), 3.94 (d, J = 8.4 Hz, 2H), 3.86 (d, J = 8.4 Hz, 2H), 3.17 (s, 2H), 1.76 (d, J = 6.4 Hz, 3H), 1.52 (s, 3H), 1.10 (s, 6H). |
| Example 94 4-[1-[5-[5-[(1R)-1-(3,5- dichloro-4- pyridyl)ethoxy]-1H- indazol-3-yl]-2- pyridyl]-3-methyl- azetidin-3- yl]morpholine | 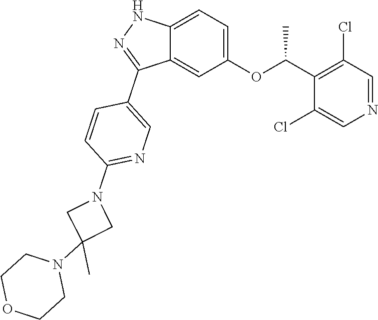 [SMILES: C[C@@H](Oc1ccc2[nH]nc(-c3ccc(N4CC(C)(N5CCOCC5)C4)nc3)c2c1)c1c(Cl)cncc1Cl] | LCMS m/z = 539.2 (M + H); 1H NMR (400 MHz, DMSO-d6) δ = 12.98 (s, 1H), 8.59 (s, 2H), 8.51 (d, J = 1.8 Hz, 1H), 7.85 (dd, J = 2.3, 8.6 Hz, 1H), 7.45 (d, J = 9.0 Hz, 1H), 7.16 (s, 1H), 7.09 (dd, J = 2.3, 8.9 Hz, 1H), 6.52 (d, J = 8.3 Hz, 1H), 6.11 (q, J = 6.7 Hz, 1H), 3.82 (d, J = 7.9 Hz, 2H), 3.69 (d, J = 7.8 Hz, 2H), 3.65-3.56 (m, 4H), 2.43 (br s, 4H), 1.76 (d, J = 6.6 Hz, 3H), 1.34 (s, 3H) |
| Example 95 (R)-N-(1-(5-(5-(1-(3,5- dichloropyridin-4- yl)ethoxy)-1H-indazol- 3-yl)pyridin-2-yl)-3- methylazetidin-3-yl)-2- (dimethylamino)acetamide | 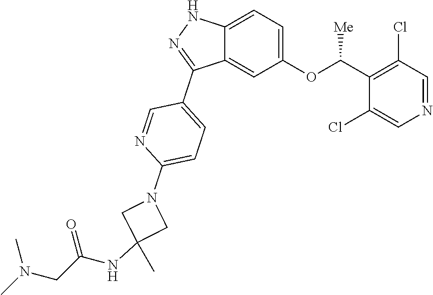 [SMILES: C[C@@H](Oc1ccc2[nH]nc(-c3ccc(N4CC(C)(NC(=O)CN(C)C)C4)nc3)c2c1)c1c(Cl)cncc1Cl] | LCMS m/z = 554.3 (M + H); 1H NMR (400 MHz, DMSO-d6) δ 12.99 (s, 1H), 8.56 (s, 2H), 8.53- 8.49 (m, 1H), 8.25 (s, 1H), 7.84 (dd, J = 8.4, 2.0 Hz, 1H), 7.45 (d, J = 9.2 Hz, 1H), 7.16 (s, 1H), 7.09 (dd, J = 9.2 Hz, 1.6 Hz, 1H), 6.54 (d, J = 8.4 Hz, 1H), 6.11 (q, J = 6.4 Hz, 1H), 4.08 (d, J = 8.0 Hz, 2H), 3.90 (d, J = 8.0 Hz, 2H), 2.87 (s, 2H), 2.21 (s, 6H), 1.76 (d, J = 6.8 Hz, 3H), 1.58 (s, 3H) |
| Example 96 (R)-1′-(5-(5-(1-(3,5- dichloropyridin-4- yl)ethoxy)-1H-indazol- 3-yl)-3-fluoropyridin- 2-yl)-3,3′-dimethyl- [1,3′-biazetidin]-3-ol | 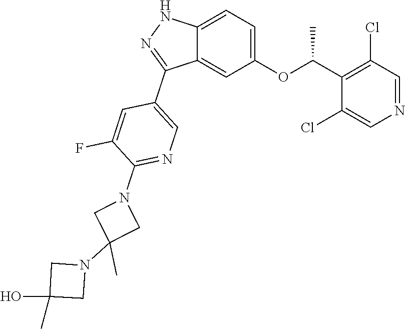 [SMILES: C[C@@H](Oc1ccc2[nH]nc(-c3cnc(N4CC(C)(N5CC(C)(O)C5)C4)c(F)c3)c2c1)c1c(Cl)cncc1Cl] | LCMS m/z = 557.2 (M + H); 1H NMR (400 MHz, DMSO-d6) δ 13.12 (s, 1H), 8.57 (s, 2H), 8.37 (s, 1H), 7.72 (dd, J = 13.2 Hz, 2.0 Hz, 1H), 7.48 (d, J = 8.8 Hz, 1H), 7.20 (d, J = 1.2 Hz, 1H), 7.11 (dd, J = 8.8 Hz, 2.0 Hz, 1H), 6.15 (q, J = 6.4 Hz, 1H), 5.22 (s, 1H), 4.21 (d, J = 8.4 Hz, 2H), 3.79 (d, J = 7.6 Hz, 2H), 3.13-3.10 (m, 4H), 1.75 (d, J = 6.4 Hz, 3H), 1.38 (s, 3H), 1.30 (s, 3H) |
| Example 97 (R)-5-(1-(3,5- dichloropyridin-4- yl)ethoxy)-3-(5-fluoro- 6-(3-methoxy-3,3′- dimethyl-[1,3′- biazetidin]-1′- yl)pyridin-3-yl)-1H- indazole | 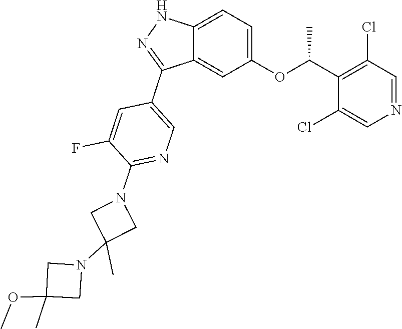 [SMILES: COC1(C)CN(C2(C)CN(c3ncc(-c4n[nH]c5ccc(O[C@H](C)c6c(Cl)cncc6Cl)cc45)cc3F)C2)C1] | LCMS m/z = 571.3 (M + H); 1H NMR (400 MHz, DMSO-d6) δ 13.12 (s, 1H), 8.56 (s, 2H), 8.37 (s, 1H), 7.70 (dd, J = 13.2 Hz, 1.2 Hz, 1H), 7.47 (d, J = 9.2 Hz, 1H), 7.19 (s, 1H), 7.09 (dd, J = 9.2 Hz, 2.4 Hz, 1H), 6.13 (q, J = 6.4 Hz, 1H), 4.12 (d, J = 8.4 Hz, 2H), 3.80 (d, J = 8.4 Hz, 2H), 3.19-3.17 (m, 2H), 3.12-3.08 (m, 5H), 1.75 (d, J = 6.4 Hz, 3H), 1.40 (s, 3H), 1.31 (s, 3H) |
| Example 98 (R)-5-(5-(1-(3,5- dichloropyridin-4- yl)ethoxy)-1H-indazol- 3-yl)-2-(3-((2- isopropoxyethyl)amino)- 3-methylazetidin-1- yl)nicotinonitrile | 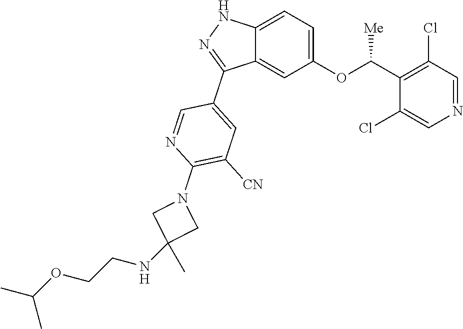 [SMILES: CC(C)OCCNC1(C)CN(c2ncc(-c3n[nH]c4ccc(O[C@H](C)c5c(Cl)cncc5Cl)cc34)cc2C#N)C1] | LCMS m/z = 580.4 (M + H); 1H NMR (400 MHz, DMSO-d6) δ 13.17 (s, 1H), 8.76 (d, J = 2.4 Hz, 1H), 8.59-8.56 (m, 2H), 8.13 (d, J = 2.4 Hz, 1H), 7.49 (d, J = 8.8 Hz, 1H), 7.15 (d, J = 2.0 Hz, 1H), 7.10 (dd, J = 11.2 Hz, 2.4 Hz, 1H), 6.14 (q, J = 6.8 Hz, 1H), 4.14-4.12 (m, 2H), 4.06-4.03 (m, 2H), 3.60-3.53 (m, 1H), 3.44-3.42 (m, 2H), 2.74- 2.67 (m, 2H), 1.76 (d, J = 6.8 Hz, 3H), 1.44 (s, 3H), 1.10 (d, J = 6.0 Hz, 6H) |
| Example 99 (R)-5-(5-(1-(3,5- dichloropyridin-4- yl)ethoxy)-6-methoxy- 1H-indazol-3-yl)-2-(3- hydroxy-3,3′-dimethyl- [1,3′-biazetidin]-1′- yl)nicotinonitrile | 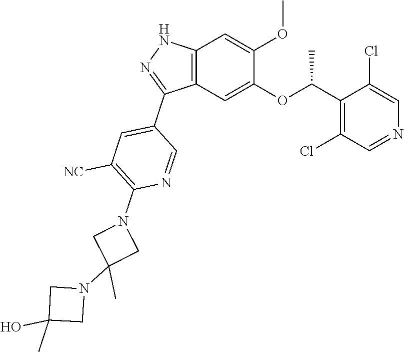 [SMILES: COc1cc2[nH]nc(-c3cnc(N4CC(C)(N5CC(C)(O)C5)C4)c(C#N)c3)c2cc1O[C@H](C)c1c(Cl)cncc1Cl] | LCMS m/z = 594.3 (M + H); 1H NMR (400 MHz, DMSO-d6) δ 12.97 (s, 1H), 8.72 (d, J = 2.0 Hz, 1H), 8.59 (s, 2H), 8.07 (d, J = 2.8 Hz, 1H), 7.04 (s, 1H), 7.01 (s, 1H), 6.00 (q, J = 6.8 Hz, 1H), 5.28 (s, 1H), 4.32 (d, J = 9.2 Hz, 2H), 3.99- 3.90 (m, 2H), 3.86 (s, 3H), 3.20- 3.05 (m, 4H), 1.76 (d, J = 6.8 Hz, 3H), 1.38 (s, 3H), 1.31 (s, 3H) |
| Example 100 (R)-5-(5-(1-(3,5- dichloropyridin-4- yl)ethoxy)-1H-indazol- 3-yl)-2-(3-hydroxy- 3,3′-dimethyl-[1,3′- biazetidin]-1′- yl)nicotinonitrile | 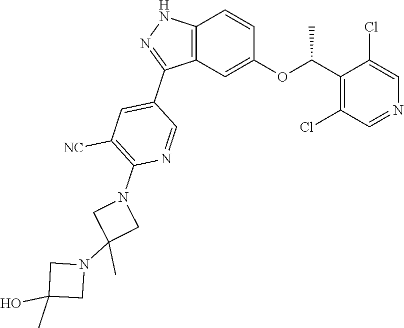 [SMILES: C[C@@H](Oc1ccc2[nH]nc(-c3cnc(N4CC(C)(N5CC(C)(O)C5)C4)c(C#N)c3)c2c1)c1c(Cl)cncc1Cl] | LCMS m/z = 564.3 (M + H); 1H NMR (400 MHz, DMSO-d6) δ 13.17 (s, 1H), 8.77 (d, J = 2.0 Hz, 1H), 8.58 (s, 2H), 8.15 (d, J = 2.4 Hz, 1H), 7.49 (d, J = 8.8 Hz, 1H), 7.16 (d, J = 2.0 Hz, 1H), 7.10 (dd, J = 9.2 Hz, 2.4 Hz, 1H), 6.14 (q, J = 6.8 Hz, 1H), 5.29 (s, 1H), 4.34 (d, J = 9.2 Hz, 2H), 3.95 (dd, J = 9.6 Hz, 2.8 Hz, 2H), 3.21-3.06 (m, 4H), 1.76 (d, J = 6.4 Hz, 3H), 1.38 (s, 3H), 1.31 (s, 3H) |
| Example 101 (R)-5-(5-(1-(3,5- dichloropyridin-4- yl)ethoxy)-6-methoxy- 1H-indazol-3-yl)-2-(3- methoxy-3,3′-dimethyl- [1,3′-biazetidin]-1′- yl)nicotinonitrile | 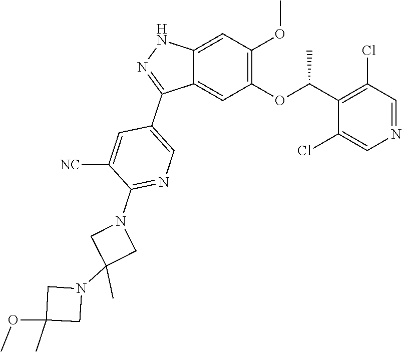 | LCMS m/z = 440.3 (M + H); 1H NMR (400 MHz, DMSO-d6) δ 12.98 (s, 1H), 8.72 (d, J = 2.4 Hz, 1H), 8.59 (s, 2H), 8.08 (d, J = 2.4 Hz, 1H), 7.04 (s, 1H), 7.01 (s, 1H), 6.00 (q, J = 6.8 Hz, 1H), 4.24 (d, J = 9.2 Hz, 2H), 3.96 (d, J = 8.8 Hz, 2H), 3.86 (s, 3H), 3.23-3.13 (m, 7H), 1.76 (d, J = 6.4 Hz, 3H), 1.42 (s, 3H), 1.32 (s, 3H) |
| Example 102 (R)-5-(5-(1-(3,5- dichloropyridin-4- yl)ethoxy)-1H-indazol- 3-yl)-2-(3-methoxy- 3,3′-dimethyl-[1,3′- biazetidin]-1′- yl)nicotinonitrile | 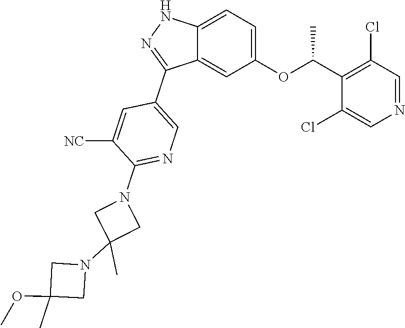 [SMILES: COC1(C)CN(C2(C)CN(c3ncc(-c4n[nH]c5ccc(O[C@H](C)c6c(Cl)cncc6Cl)cc45)cc3C#N)C2)C1] | LCMS m/z = 578.3 (M + H); 1H NMR (400 MHz, DMSO-d6) δ 13.18 (s, 1H), 8.77 (d, J = 2.4 Hz, 1H), 8.58 (s, 2H), 8.15 (d, J = 2.0 Hz, 1H), 7.48 (d, J = 5.2 Hz, 1H), 7.17 (d, J = 1.6 Hz, 1H), 7.11 (dd, J = 8.8 Hz, 2.0 Hz, 1H), 6.14 (q, J = 6.8 Hz, 1H), 4.25 (d, J = 6.8 Hz, 2H), 3.97 (d, J = 9.2 Hz, 2H), 3.23- 3.13 (m, 7H), 1.76 (d, J = 6.4 Hz, 3H), 1.42 (s, 3H), 1.33 (s, 3H) |
| Example 103 (R)-5-(1-(3,5- dichloropyridin-4- yl)ethoxy)-3-(5-fluoro- 6-(3′-methyl-[1,3′- biazetidin]-1′- yl)pyridin-3-yl)-1H- indazole | 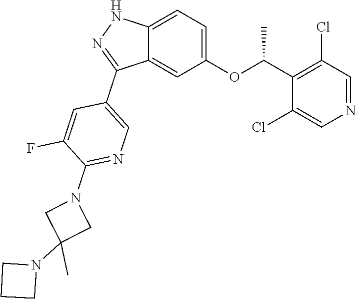 [SMILES: C[C@@H](Oc1ccc2[nH]nc(-c3cnc(N4CC(C)(N5CCC5)C4)c(F)c3)c2c1)c1c(Cl)cncc1Cl] | LCMS m/z = 527.2 (M + H); 1H NMR (400 MHz, DMSO-d6) δ 13.12 (s, 1H), 8.57 (s, 2H), 8.37 (s, 1H), 7.70 (dd, J = 13.2 Hz, 2.0 Hz, 1H), 7.47 (d, J = 8.8 Hz, 1H), 7.20 (d, J = 1.6 Hz, 1H), 7.10 (dd, J = 9.2 Hz, 2.0 Hz, 1H), 6.14 (q, J = 6.4 Hz, 1H), 4.19 (d, J = 8.8 Hz, 2H), 3.77 (d, J = 7.6 Hz, 2H), 3.28-3.24 (m, 4H), 2.03-1.90 (m, 2H), 1.76 (d, J = 6.8 Hz, 3H), 1.28 (s, 3H) |
| Example 104 (R)-1-((1-(5-(5-((R)-1- (3,5-dichloropyridin-4- yl)ethoxy)-1H-indazol- 3-yl)-3-fluoropyridin- 2-yl)-3-methylazetidin- 3-yl)amino)propan-2-ol | 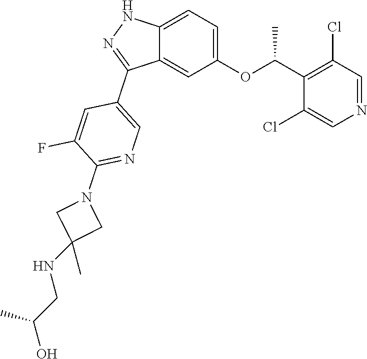 | LCMS m/z = 440.3 (M + H); 1H NMR (400 MHz, DMSO-d6) δ 13.11 (s, 1H), 8.57 (s, 2H), 8.37 (s, 1H), 7.71 (dd, J = 13.2 Hz, 2.0 Hz, 1H), 7.48 (d, J = 9.2 Hz, 1H), 7.20 (d, J = 1.6 Hz, 1H), 7.11 (dd, J = 9.2 Hz, 2.4 Hz, 1H), 6.17 (q, J = 6.4 Hz, 1H), 4.53 (s, 1H), 4.01-3.98 (m, 2H), 3.89-3.87 (m, 2H), 3.65- 3.62 (m, 1H), 2.47-2.42 (m, 2H), 1.77 (d, J = 6.4 Hz, 3H), 1.43 (s, 3H), 1.09 (d, J = 6.4 Hz, 3H) |
| Example 105 (R)-5-(5-(1-(3,5- dichloropyridin-4- yl)ethoxy)-1H-indazol- 3-yl)-2-(3-methyl-3- ((methylsulfonyl)methyl) azetidin-1- yl)nicotinonitrile | 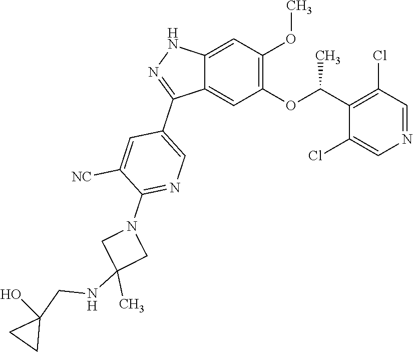 [SMILES: COc1cc2[nH]nc(-c3cnc(N4CC(C)(NCC5(O)CC5)C4)c(C#N)c3)c2cc1O[C@H](C)c1c(Cl)cncc1Cl] | LCMS m/z = 594.3 (M + H); 1H NMR (400 MHz, DMSO-d6) δ 12.96 (s, 1H), 8.72 (s, 1H), 8.59 (d, J = 2.4 Hz, 2H), 8.06 (s, 1H), 7.04 (s, 1H), 7.01-6.98 (m, 1H), 6.00 (q, J = 5.6 Hz, 1H), 5.20 (brs, 1H), 4.18-4.16 (m, 2H), 4.06-4.02 (m, 2H), 3.86 (s, 3H), 2.67-2.66 (m, 2H), 1.76 (d, J = 6.4 Hz, 3H), 1.44 (s, 3H), 0.55-0.48 (m, 4H) |
| Example 106 (R)-N- (cyclopropylmethyl)-1- (5-(5-(1-(3,5- dichloropyridin-4- yl)ethoxy)-1H-indazol- 3-yl)-3-fluoropyridin- 2-yl)-3-methylazetidin- 3-amine | 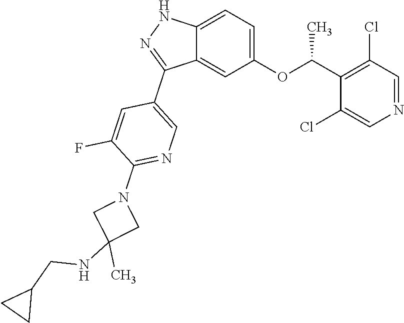 [SMILES: C[C@@H](Oc1ccc2[nH]nc(-c3cnc(N4CC(C)(NCC5CC5)C4)c(F)c3)c2c1)c1c(Cl)cncc1Cl] | LCMS m/z = 541.3 (M + H); 1H NMR (400 MHz, DMSO- d6) δ 13.12 (s, 1H), 8.57 (s, 2H), 8.37 (d, J = 1.6 Hz, 1H), 7.69 (dd, J = 13.2 Hz, 2.0 Hz, 1H), 7.48 (d, J = 8.8 Hz, 1H), 7.20 (d, J = 2.0 Hz, 1H), 7.10 (dd, J = 8.8 Hz, 2.4 Hz, 1H), 6.14 (q, J = 6.8 Hz, 1H), 4.02 (d, J = 8.4 Hz, 2H), 3.87 (d, J = 6.8 Hz, 2H), 2.44 (d, J = 6.4 Hz, 2H), 1.76 (d, J = 6.4 Hz, 3H), 1.44 (s, 3H), 0.91-0.81 (m, 1H), 0.45-0.40 (m, 2H), 0.19-0.12 (m, 2H) |
| Example 107 (R)-5-(1-(3,5- dichloropyridin-4- yl)ethoxy)-3-(5-fluoro- 6-(3-methyl-3- (pyrrolidin-1- yl)azetidin-1- yl)pyridin-3-yl)-1H- indazole | 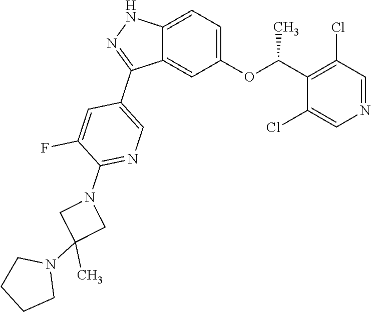 [SMILES: C[C@@H](Oc1ccc2[nH]nc(-c3cnc(N4CC(C)(N5CCCC5)C4)c(F)c3)c2c1)c1c(Cl)cncc1Cl] | LCMS m/z = 541.3 (M + H); 1H NMR (400 MHz, DMSO-d6) δ 13.11 (s, 1H), 8.57 (s, 2H), 8.36 (s, 1H), 7.71 (dd, J = 13.2 Hz, 1.6 Hz, 1H), 7.48 (d, J = 9.2 Hz, 1H), 7.20 (d, J = 1.6 Hz, 1H), 7.11 (dd, J = 8.8 Hz, 2.0 Hz, 1H), 6.16 (q, J = 6.4 Hz, 1H), 4.18 (d, J = 8.0 Hz, 2H), 3.83 (d, J = 8.0 Hz, 2H), 2.68- 2.63 (m, 4H), 1.77 (d, J = 8.0 Hz, 3H), 1.74-1.72 (m, 4H), 1.44 (s, 3H) |
| Example 108 (R)-N-((1-(5-(5-(1- (3,5-dichloropyridin-4- yl)ethoxy)-1H-indazol- 3-yl)-3-fluoropyridin- 2-yl)-3-methylazetidin- 3-yl)methyl)propan-2- amine | 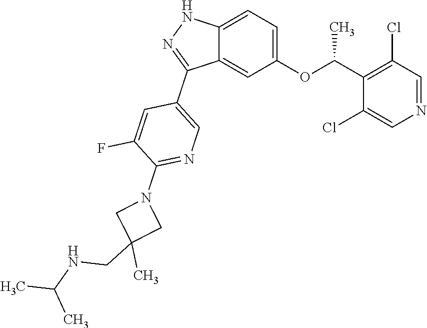 [SMILES: CC(C)NCC1(C)CN(c2ncc(-c3n[nH]c4ccc(O[C@H](C)c5c(Cl)cncc5Cl)cc34)cc2F)C1] | LCMS m/z = 543.2 (M + H); 1H NMR (400 MHz, DMSO-d6) δ 13.10 (s, 1H), 8.57 (s, 2H), 8.35 (t, J = 1.6 Hz, 1H), 7.67 (dd, J = 13.2 Hz, 1.6 Hz, 1H), 7.47 (d, J = 9.2 Hz, 1H), 7.19 (d, J = 2.0 Hz, 1H), 7.10 (dd, J = 8.8 Hz, 2.0 Hz, 1H), 6.14 (q, J = 6.4 Hz, 1H), 3.97 (dd, J = 8.0 Hz, 1.6 Hz, 2H), 3.74 (dd, J = 8.4 Hz, 2.0 Hz, 2H), 2.78-2.64 (m, 3H), 1.76 (d, J = 6.4 Hz, 3H), 1.30 (s, 3H), 1.01 (d, J = 6.4 Hz, 6H) |
| Example 109 (R)-4-((1-(5-(5-(1-(3,5- dichloropyridin-4- yl)ethoxy)-1H-indazol- 3-yl)-3-fluoropyridin- 2-yl)-3-methylazetidin- 3- yl)methyl)morpholine | 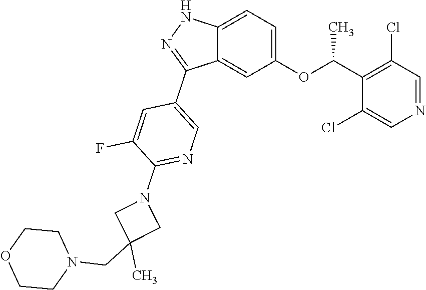 [SMILES: C[C@@H](Oc1ccc2[nH]nc(-c3cnc(N4CC(C)(CN5CCOCC5)C4)c(F)c3)c2c1)c1c(Cl)cncc1Cl] | LCMS m/z = 571.2 (M + H); 1H NMR (400 MHz, DMSO-d6) δ 13.11 (s, 1H), 8.57 (s, 2H), 8.36 (t, J = 1.6 Hz, 1H), 7.69 (dd, J = 13.2 Hz, 1.6 Hz, 1H), 7.47 (d, J = 8.8 Hz, 1H), 7.19 (d, J = 1.6 Hz, 1H), 7.10 (dd, J = 9.2 Hz, 2.0 Hz, 1H), 6.13 (q, J = 6.8 Hz, 1H), 3.92 (d, J = 7.2 Hz, 2H), 3.81 (dd, J = 8.0 Hz, 1.6 Hz, 2H), 3.57 (t, J = 4.4 Hz, 4H), 2.56-2.52 (m, 2H), 2.35 (t, J = 4.0 Hz, 4H), 1.76 (d, J = 6.8 Hz, 3H), 1.39 (s, 3H) |
| Example 110 (R)-5-(1-(3,5- dichloropyridin-4- yl)ethoxy)-3-(5-fluoro- 6-(3-methyl-3- (pyrrolidin-1- ylmethyl)azetidin-1- yl)pyridin-3-yl)-1H- indazole | 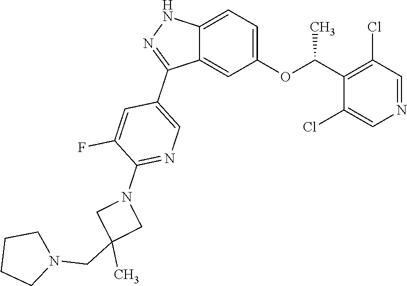 [SMILES: C[C@@H](Oc1ccc2[nH]nc(-c3cnc(N4CC(C)(CN5CCCC5)C4)c(F)c3)c2c1)c1c(Cl)cncc1Cl] | LCMS m/z = 555.2 (M + H); 1H NMR (400 MHz, DMSO-d6) δ 13.11 (brs, 1H), 8.57 (s, 2H), 8.35 (t, J = 1.6 Hz, 1H), 7.68 (dd, J = 13.2 Hz, 1.6 Hz, 1H), 7.48 (d, J = 9.2 Hz, 1H), 7.19 (d, J = 2.0 Hz, 1H), 7.09 (dd, J = 9.2 Hz, 2.0 Hz, 1H), 6.14 (q, J = 6.4 Hz, 1H), 3.93 (d, J = 7.6 Hz, 2H), 3.80 (dd, J = 8.0 Hz, 1.6 Hz, 2H), 2.63 (s, 2H), 2.48-2.40 (m, 4H), 1.76 (d, J = 6.4 Hz, 3H), 1.72-1.64 (m, 4H), 1.38 (s, 3H) |
| Example 111 (R)-5-(1-(3,5- dichloropyridin-4- yl)ethoxy)-3-(5-fluoro- 6-(3,3,3′-trimethyl- [1,3′-biazetidin]-1′- yl)pyridin-3-yl)-1H- indazole | 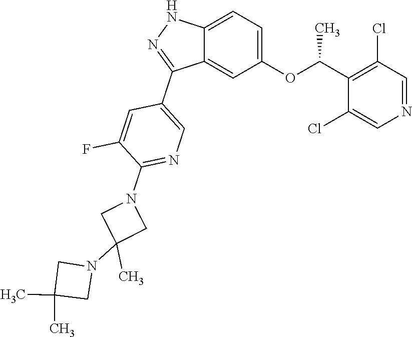 [SMILES: C[C@@H](Oc1ccc2[nH]nc(-c3cnc(N4CC(C)(N5CC(C)(C)C5)C4)c(F)c3)c2c1)c1c(Cl)cncc1Cl] | LCMS m/z = 555.3 (M + H); 1H NMR (400 MHz, DMSO-d6) δ 13.11 (s, 1H), 8.57 (s, 2H), 8.37 (s, 1H), 7.72 (d, J = 12.8 Hz, 1H), 7.48 (d, J = 9.2 Hz, 1H), 7.20 (d, J = 1.6 Hz, 1H), 7.11 (dd, J = 8.8 Hz, 2.0 Hz, 1H), 6.17 (q, J = 6.8 Hz, 1H), 4.15 (d, J = 8.4 Hz, 2H), 3.79 (d, J = 7.6 Hz, 2H), 3.05-2.99 (m, 4H), 1.77 (d, J = 6.4 Hz, 3H), 1.29 (s, 3H), 1.20 (s, 6H) |
| Example 112 (S)-1-((1-(5-(5-((R)-1- (3,5-dichloropyridin-4- yl)ethoxy)-1H-indazol- 3-yl)-3-fluoropyridin- 2-yl)-3-methylazetidin- 3-yl)amino)propan-2-ol | 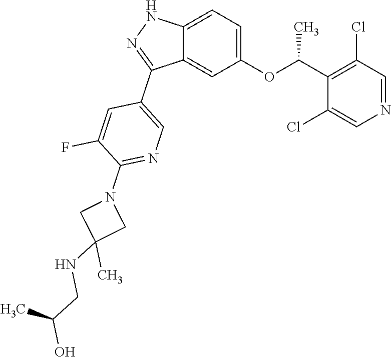 [SMILES: C[C@H](O)CNC1(C)CN(c2ncc(-c3n[nH]c4ccc(O[C@H](C)c5c(Cl)cncc5Cl)cc34)cc2F)C1] | LCMS m/z = 545.2 (M + H); 1H NMR (400 MHz, DMSO-d6) δ 13.11 (s, 1H), 8.57 (s, 2H), 8.37 (s, 1H), 7.69 (dd, J = 13.2 Hz, 1.6 Hz, 1H), 7.47 (d, J = 8.8 Hz, 1H), 7.20 (d, J = 1.2 Hz, 1H), 7.10 (dd, J = 8.8 Hz, 1.6 Hz, 1H), 6.14 (q, J = 6.4 Hz, 1H), 4.52 (d, J = 3.6 Hz, 1H), 3.99 (t, J = 7.2 Hz, 2H), 3.88 (dd, J = 8.4 Hz, 1.6 Hz, 2H), 3.68-3.59 (m, 1H), 2.49-2.42 (m, 2H), 1.76 (d, J = 6.4 Hz, 3H), 1.43 (s, 3H), 1.08 (d, J = 6.4 Hz, 3H) |
| Example 113 (R)-1-((1-(5-(5-(1-(3,5- dichloropyridin-4- yl)ethoxy)-1H-indazol- 3-yl)-3-fluoropyridin- 2-yl)-3-methylazetidin- 3-yl)amino)-2- methylpropan-2-ol | 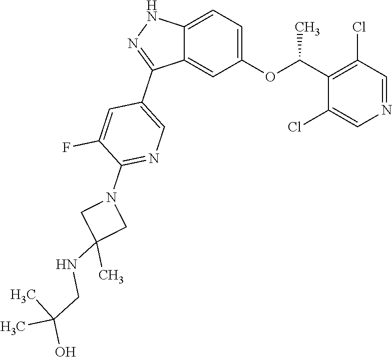 [SMILES: C[C@@H](Oc1ccc2[nH]nc(-c3cnc(N4CC(C)(NCC(C)(C)O)C4)c(F)c3)c2c1)c1c(Cl)cncc1Cl] | LCMS m/z = 559.3 (M + H); 1H NMR (400 MHz, DMSO-d6) δ 13.11 (s, 1H), 8.56 (s, 2H), 8.36 (s, 1H), 7.70 (d, J = 13.2 Hz, 1H), 7.46 (dd, J = 8.8 Hz, 1.2 Hz, 1H), 7.19 (s, 1H), 7.11 (d, J = 8.8 Hz, 1H), 6.13 (q, J = 6.0 Hz, 1H), 4.21 (s, 1H), 3.98 (d, J = 8.0 Hz, 2H), 3.87 (d, J = 8.0 Hz, 2H), 2.55-2.52 (m, 1H), 2.42 (s, 2H), 1.75 (d, J = 6.4 Hz, 3H), 1.43 (s, 3H), 1.11 (s, 6H) |
| Example 114 (R)-2-((1-(5-(5-(1-(3,5- dichloropyridin-4- yl)ethoxy)-1H-indazol- 3-yl)-3-fluoropyridin- 2-yl)-3-methylazetidin- 3-yl)amino)ethan-1-ol | 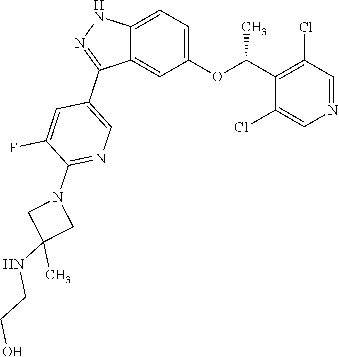 [SMILES: C[C@@H](Oc1ccc2[nH]nc(-c3cnc(N4CC(C)(NCCO)C4)c(F)c3)c2c1)c1c(Cl)cncc1Cl] | LCMS m/z = 531.3 (M + H); 1H NMR (400 MHz, DMSO-d6) δ 13.11 (s, 1H), 8.57 (s, 2H), 8.37 (t, J = 1.6 Hz, 1H), 7.69 (dd, J = 13.2 Hz, 1.6 Hz, 1H), 7.47 (d, J = 9.2 Hz, 1H), 7.20 (d, J = 2.0 Hz, 1H), 7.10 (dd, J = 9.2 Hz, 2.8 Hz, 1H), 6.14 (q, J = 6.4 Hz, 1H), 4.58-4.53 (m, 1H), 3.99 (d, J = 8.0 Hz, 2H), 3.89 (dd, J = 8.0 Hz, 2.0 Hz, 2H), 3.47 (q, J = 5.6 Hz, 2H), 2.63 (t, J = 5.6 Hz, 2H), 1.76 (d, J = 6.4 Hz, 3H), 1.44 (s, 3H) |
| Example 115 (R)-1-((1-(5-(5-(1-(3,5- dichloropyridin-4- yl)ethoxy)-1H-indazol- 3-yl)pyridin-2-yl)-3- methylazetidin-3- yl)amino)-2- methylpropan-2-ol | 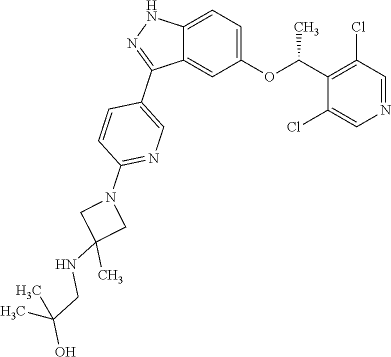 [SMILES: C[C@@H](Oc1ccc2[nH]nc(-c3ccc(N4CC(C)(NCC(C)(C)O)C4)nc3)c2c1)c1c(Cl)cncc1Cl] | LCMS m/z = 541.3 (M + H); 1H NMR (400 MHz, DMSO-d6) δ 12.98 (s, 1H), 8.60 (s, 2 H), 8.50 (d, J = 2.4 Hz, 1H), 7.83 (dd, J = 8.4 Hz, 2.0 Hz, 1H), 7.45 (d, J = 9.2 Hz, 1H), 7.16 (s, 1H), 7.09 (dd, J = 8.8 Hz, 2.0 Hz, 1H), 6.52 (d, J = 8.8 Hz, 1H), 6.11 (q, J = 6.8 Hz, 1H), 4.21 (s, 1H), 3.83 (d, J = 8.0 Hz, 2H), 3.75 (d, J = 7.6 Hz, 2H), 2.50- 2.43 (m, 2 H), 1.76 (d, J = 6.8 Hz, 3H), 1.42 (s, 3 H), 1.11 (s, 6 H) |
| Example 116 3-[6-[3-(azetidin-1-yl)- 3-methyl-azetidin-1- yl]-3-pyridyl]-5-[(1R)- 1-(3,5-dichloro-4- pyridyl)ethoxy]-1H- indazole | 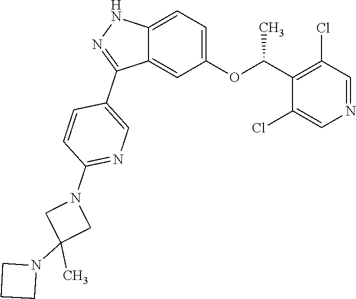 [SMILES: C[C@@H](Oc1ccc2[nH]nc(-c3ccc(N4CC(C)(N5CCC5)C4)nc3)c2c1)c1c(Cl)cncc1Cl] | LCMS m/z = 509.1 (M +H); 1H NMR (400 MHz, DMSO-d6) δ = 13.06 (s, 1H), 8.61 - 8.55 (m, 3H), 7.93 (dd, J = 2.3, 8.6 Hz, 1H), 7.48 (d, J = 9.0 Hz, 1H), 7.17 (s, 1H), 7.11 (dd, J = 2.3, 9.0 Hz, 1H), 6.64 (d, J = 8.6 Hz, 1H), 6.12 (q, J = 6.6 Hz, 1H), 4.27 (br d, J = 9.7 Hz, 2H), 4.03 (br s, 3H), 3.93 (br d, J = 9.5 Hz, 3H), 2.48-2.23 (m, 2H), 1.77 (d, J = 6.7 Hz, 3H), 1.49 (s, 3H) |
| Example 117 (R)-1-(5-(5-(1-(3,5- dichloropyridin-4- yl)ethoxy)-1H-indazol- 3-yl)-3-fluoropyridin- 2-yl)-N-isopropyl-3- methylazetidin-3-amine | 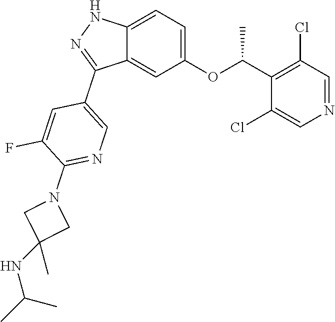 [SMILES: CC(C)NC1(C)CN(c2ncc(-c3n[nH]c4ccc(O[C@H](C)c5c(Cl)cncc5Cl)cc34)cc2F)C1] | LCMS m/z = 529.3 (M + H); 1H NMR (400 MHz, DMSO-d6) δ 13.19 (s, 1H), 9.15 (brs, 1H), 8.57 (s, 2H), 8.43 (s, 1H), 7.81 (dd, J = 13.2 Hz, 1.6 Hz, 1H), 7.49 (d, J = 1.6 Hz, 1H), 7.20 (d, J = 1.6 Hz, 1H), 7.11 (dd, J = 8.8 Hz, 2.4 Hz, 1H), 6.15 (q, J = 6.8 Hz, 1H), 4.32 (d, J = 7.6 Hz, 2H), 4.14 (d, J = 9.2 Hz, 2H), 3.56-3.45 (m, 1H), 1.76 (d, J = 6.8 Hz, 3H), 1.72 (s, 3H), 1.23 (d, J = 5.2 Hz, 6H) |
| Example 118 (R)-1-(((1-(5-(5-(1- (3,5-dichloropyridin-4- yl)ethoxy)-1H-indazol- 3-yl)-3-fluoropyridin- 2-yl)-3-methylazetidin- 3- yl)amino)methyl)cyclo- propan-1-ol | 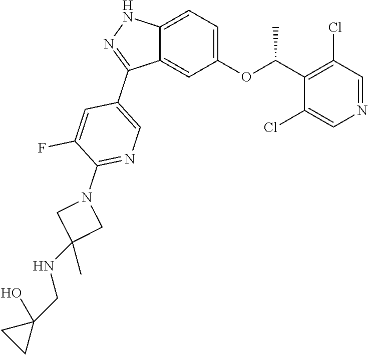 [SMILES: C[C@@H](Oc1ccc2[nH]nc(-c3cnc(N4CC(C)(NCC5(O)CC5)C4)c(F)c3)c2c1)c1c(Cl)cncc1Cl] | LCMS m/z = 557.2 (M + H); 1H NMR (400 MHz, DMSO-d6) δ 13.11 (s, 1H), 8.59-8.55 (m, 2H), 8.37 (s, 1H), 7.70 (dd, J = 13.2 Hz, 1.2 Hz, 1H), 7.47 (d, J = 8.8 Hz, 1H), 7.20 (s, 1H), 7.10 (dd, J = 11.2 Hz, 2.4 Hz, 1H), 6.14 (q, J = 6.8 Hz, 1H), 5.19 (s, 1H), 4.08-3.97 (m, 2H), 3.92-3.88 (m, 2H), 2.72-2.65 (m, 2H), 1.76 (d, J = 6.4 Hz, 3H), 1.44 (s, 3H), 0.58-0.52 (m, 2H), 0.51-0.44 (m, 2H) |
| Example 119 (R)-1-(((1-(5-(5-(1- (3,5-dichloropyridin-4- yl)ethoxy)-6-methoxy- 1H-indazol-3-yl)-3- fluoropyridin-2-yl)-3- methylazetidin-3- yl)amino)methyl)cyclo- propan-1-ol | 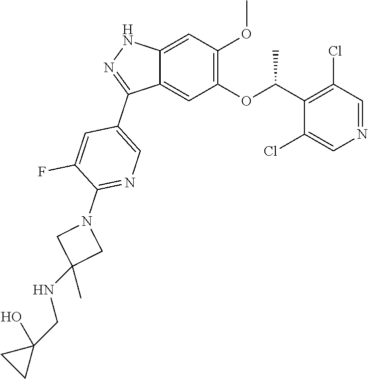 [SMILES: COc1cc2[nH]nc(-c3cnc(N4CC(C)(NCC5(O)CC5)C4)c(F)c3)c2cc1O[C@H](C)c1c(Cl)cncc1Cl] | LCMS m/z = 587.3 (M + H); 1H NMR (400 MHz, DMSO-d6) δ 12.92 (s, 1H), 8.59 (s, 2H), 8.30 (s, 1H), 7.64 (d, J = 13.2 Hz, 1H), 7.04 (s, 1H), 7.00 (s, 1H), 5.99 (q, J = 6.8 Hz, 1H), 5.21 (brs, 1H), 4.03- 4.01 (m, 2H), 3.89-3.86 (m, 5H), 2.67-2.66 (m, 2H), 1.76 (d, J = 6.8 Hz, 3H), 1.44 (s, 3H), 0.54-0.47 (m, 4H) |
| Example 120 (R)-5-(5-(1-(3,5- dichloropyridin-4- yl)ethoxy)-1H-indazol- 3-yl)-2-(3-(((1- hydroxycyclopropyl)meth- yl)amino)-3- methylazetidin-1- yl)nicotinonitrile | 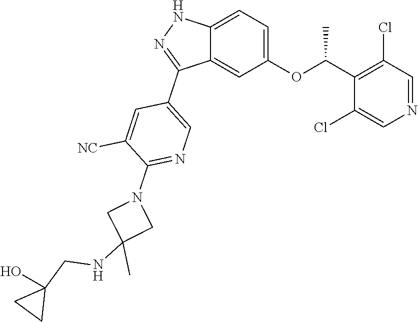 [SMILES: C[C@@H](Oc1ccc2[nH]nc(-c3cnc(N4CC(C)(NCC5(O)CC5)C4)c(C#N)c3)c2c1)c1c(Cl)cncc1Cl] | LCMS m/z = 564.2 (M + H); 1H NMR (400 MHz, DMSO-d6) δ 13.17 (s, 1H), 8.76 (d, J = 2.0 Hz, 1H), 8.60-8.56 (m, 2H), 8.13 (d, J = 2.4 Hz, 1H), 7.48 (d, J = 8.8 Hz, 1H), 7.16 (d, J = 1.6 Hz, 1H), 7.10 (dd, J = 8.8 Hz, 2.4 Hz, 1H), 6.14 (q, J = 6.4 Hz, 1H), 5.18 (s, 1H), 4.16 (d, J = 8.8 Hz, 2H), 4.06 (d, J = 8.8 Hz, 2H), 2.64 (s, 2H), 1.76 (d, J = 6.8 Hz, 3H), 1.44 (s, 3H), 0.55- 0.51 (m, 2H), 0.49-0.45 (m, 2H) |
| Example 121 (R)-((1-(5-(5-(1-(3,5- dichloropyridin-4- yl)ethoxy)-1H-indazol- 3-yl)-3-fluoropyridin- 2-yl)-3-methylazetidin- 3- yl)methyl)dimethylphos- phine oxide | 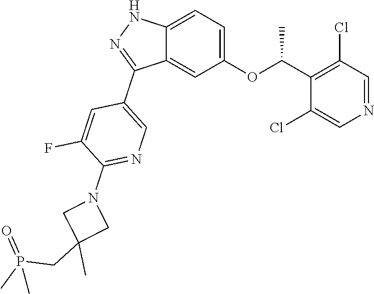 [SMILES: C[C@@H](Oc1ccc2[nH]nc(-c3cnc(N4CC(C)(CP(C)(C)=O)C4)c(F)c3)c2c1)c1c(Cl)cncc1Cl] | LCMS m/z = 562.2 (M + H); 1H NMR (400 MHz, DMSO-d6) δ 13.12 (s, 1H), 8.57 (s, 2H), 8.36 (d, J = 1.6 Hz, 1H), 7.69 (dd, J = 13.2 Hz, 2.0 Hz, 1H), 7.47 (d, J = 9.2 Hz, 1H), 7.18 (s, 1H), 7.10 (dd, J = 9.2 Hz, 2.4 Hz, 1H), 6.14 (q, J = 6.8 Hz, 1H), 4.11 (d, J = 7.2 Hz, 2H), 3.87 (d, J = 7.6 Hz, 2H), 2.20 (d, J = 11.2 Hz, 2H), 1.75 (d, J = 6.8 Hz, 3H), 1.56 (s, 3H), 1.46 (s, 3H), 1.43 (s, 3H) |
| Example 122 (R)-5-(5-(1-(3,5- dichloropyridin-4- yl)ethoxy)-1H-indazol- 3-yl)-2-(3-methyl-3- (morpholinomethyl)aze- tidin-1- yl)nicotinonitrile | 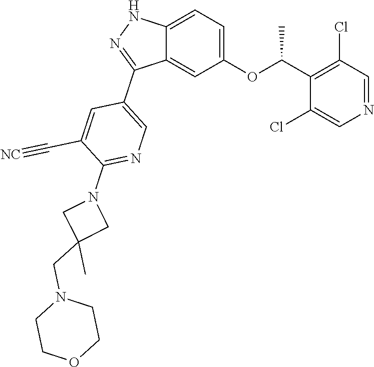 | LCMS m/z = 578.3 (M + H); 1H NMR (400 MHz, DMSO-d6) δ 13.18 (s, 1H), 8.76 (d, J = 2.4 Hz, 1H), 8.57 (s, 2H), 8.14 (d, J = 2.8 Hz, 1H), 7.50 (d, J = 9.2 Hz, 1H), 7.16 (d, J = 1.6 Hz, 1H), 7.12 (dd, J = 8.8 Hz, 2.0 Hz, 1H), 6.17 (q, J = 6.8 Hz, 1H), 4.09 (d, J = 8.4 Hz, 2H), 3.99 (d, J = 8.0 Hz, 2H), 3.58 (t, J = 4.4 Hz, 4H), 2.53 (s, 2H), 2.38 (t, J = 4.4 Hz, 4H), 1.77 (d, J = 6.8 Hz, 3H), 1.39 (s, 3H) |
| Example 123 (R)-5-(5-(1-(3,5- dichloropyridin-4- yl)ethoxy)-1H-indazol- 3-yl)-2-(3-methyl-3- (pyrrolidin-1- ylmethyl)azetidin-1- yl)nicotinonitrile | 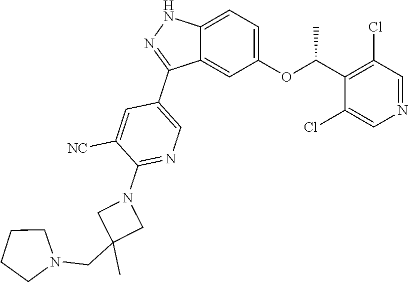 [SMILES: C[C@@H](Oc1ccc2[nH]nc(-c3cnc(N4CC(C)(CN5CCCC5)C4)c(C#N)c3)c2c1)c1c(Cl)cncc1Cl] | LCMS m/z = 562.3 (M + H); 1H NMR (400 MHz, DMSO-d6) δ 13.17 (s, 1H), 8.76 (s, 1H), 8.58 (d, J = 1.2 Hz, 2H), 8.14 (s, 1H), 7.49 (d, J = 9.2 Hz, 1H), 7.16 (s, 1H), 7.10 (d, J = 9.6 Hz, 1H), 6.14 (q, J = 6.4 Hz, 1H), 4.10-4.08 (m, 2H), 3.99-3.96 (m, 2H), 2.67-2.66 (m, 2H), 2.48-2.46 (m, 4H), 1.77- 1.70 (m, 7H), 1.39 (s, 3H) |
| Example 124 (R)-5-(5-(1-(3,5- dichloropyridin-4- yl)ethoxy)-6-fluoro- 1H-indazol-3-yl)-2-(3- methyl-3- morpholinoazetidin-1- yl)nicotinonitrile | 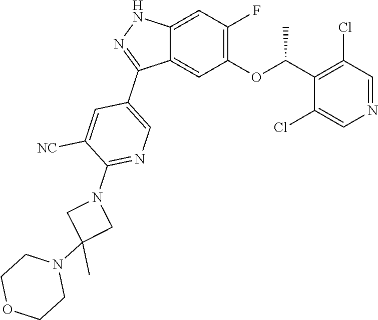 [SMILES: C[C@@H](Oc1cc2c(-c3cnc(N4CC(C)(N5CCOCC5)C4)c(C#N)c3)n[nH]c2cc1F)c1c(Cl)cncc1Cl] | LCMS m/z = 582.2 (M + H); 1H NMR (400 MHz, DMSO-d6) δ 13.26 (brs, 1H), 8.73 (d, J = 2.4 Hz, 1 H), 8.60 (s, 2H), 8.14 (d, J = 2.4 Hz, 1H), 7.47 (d, J = 11.2 Hz, 1H), 7.23 (d, J = 8.0 Hz, 1H), 6.18 (q, J = 6.4 Hz, 1H), 4.16-4.11 (m, 2 H), 3.99-3.95 (m, 2 H), 3.62-3.60 (m, 4H), 2.45-2.42 (m, 4H), 1.80 (d, J = 6.4 Hz, 3 H), 1.35 (s, 3H) |
| Example 125 1-[5-[5-[(1R)-1-(3,5- dichloro-4- pyridyl)ethoxy]-1H- indazol-3-yl]-2- pyridyl]-N-(2,2- difluoroethyl)-3- methyl-azetidin-3- amine | 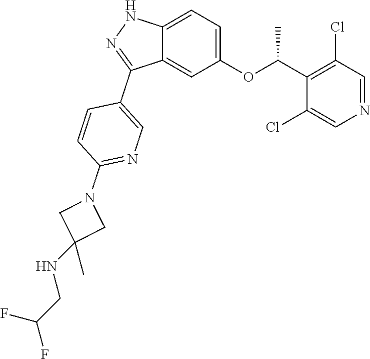 [SMILES: C[C@@H](Oc1ccc2[nH]nc(-c3ccc(N4CC(C)(NCC(F)F)C4)nc3)c2c1)c1c(Cl)cncc1Cl] | LCMS m/z = 533.1 (M + H); 1H NMR (400 MHz, DMSO-d6) δ = 12.97 (s, 1H), 8.59 (s, 2H), 8.51 (d, J = 1.7 Hz, 1H), 7.87-7.82 (m, 1H), 7.45 (d, J = 9.0 Hz, 1H), 7.17 (d, J = 2.0 Hz, 1H), 7.13-7.06 (m, 1H), 6.52 (d, J = 8.5 Hz, 1H), 6.18- 5.77 (m, 2H), 3.84 (d, J = 8.1 Hz, 2H), 3.76 (d, J = 8.1 Hz, 2H), 2.94 (ddt, J = 4.2, 7.7, 15.8 Hz, 2H), 2.79-2.66 (m, 1H), 1.76 (d, J = 6.6 Hz, 3H), 1.44 (s, 3H) |
| Example 126 (R)-N- (cyclopropylmethyl)-1- (5-(5-(1-(3,5- dichloropyridin-4- yl)ethoxy)-6-methoxy- 1H-indazol-3-yl)-3- fluoropyridin-2-yl)-3- methylazetidin-3-amine | 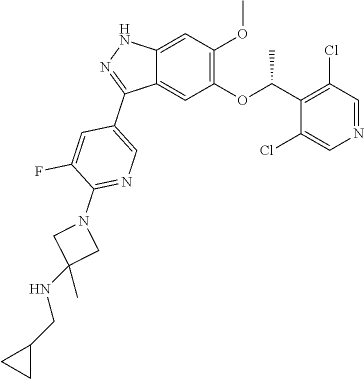 [SMILES: COc1cc2[nH]nc(-c3cnc(N4CC(C)(NCC5CC5)C4)c(F)c3)c2cc1O[C@H](C)c1c(Cl)cncc1Cl] | LCMS m/z = 571.3 (M + H); 1H NMR (400 MHz, DMSO-d6) δ 12.91 (s, 1H), 8.59 (s, 2H), 8.29 (s, 1H), 7.62 (dd, J = 13.2 Hz, 1.6 Hz, 1H), 7.08 (s, 1H), 7.00 (s, 1H), 5.99 (q, J = 6.4 Hz, 1H), 3.98 (d, J = 7.6 Hz, 2H), 3.86-3.83 (m, 5H), 2.39 (d, J = 6.8 Hz, 2H), 1.76 (d, J = 6.8 Hz, 3H), 1.42 (s, 3H), 0.87-0.82 (m, 1H), 0.43-0.39 (m, 2H), 0.15-0.13 (m, 2H) |
| Example 127 (R)-1-(5-(5-(1-(3,5- dichloropyridin-4- yl)ethoxy)-6-fluoro- 1H-indazol-3-yl)-3- fluoropyridin-2-yl)-N- isopropyl-3- methylazetidin-3-amine | 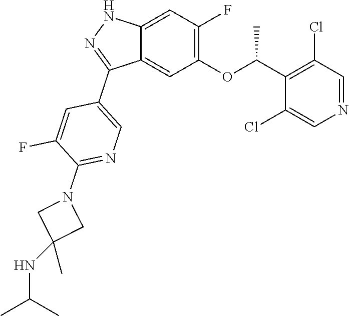 [SMILES: CC(C)NC1(C)CN(c2ncc(-c3n[nH]c4cc(F)c(O[C@H](C)c5c(Cl)cncc5Cl)cc34)cc2F)C1] | LCMS m/z = 547.3 (M + H); 1H NMR (400 MHz, DMSO-d6) δ 13.20 (s, 1H), 8.60 (s, 2H), 8.34 (s, 1H), 7.67 (dd, J = 13.2 Hz, 1.6 Hz, 1H), 7.45 (d, J = 10.8 Hz, 1H), 7.28 (d, J = 8.0 Hz, 1H), 6.17 (q, J = 6.4 Hz, 1H), 3.93-3.88 (m, 4H), 2.97- 2.91 (m, 1H), 1.80 (d, J = 6.4 Hz, 3H), 1.47 (s, 3H), 0.98 (d, J = 6.4 Hz, 6H) |
| Example 128 (R)-5-(1-(3,5- dichloropyridin-4- yl)ethoxy)-6-fluoro-3- (5-fluoro-6-(3-methyl- 3-(pyrrolidin-1- yl)azetidin-1- yl)pyridin-3-yl)-1H- indazole | 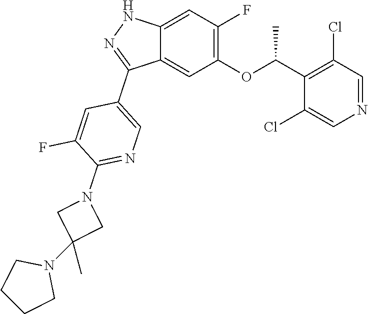 [SMILES: C[C@@H](Oc1cc2c(-c3cnc(N4CC(C)(N5CCCC5)C4)c(F)c3)n[nH]c2cc1F)c1c(Cl)cncc1Cl] | LCMS m/z = 559.2 (M + H); 1H- NMR (400 MHz, DMSO-d6) δ 13.19 (s, 1H), 8.60 (s, 2H), 8.33 (t, J = 1.6 Hz, 1H), 7.67 (dd, J = 13.2 Hz, 2.0 Hz, 1H), 7.45 (d, J = 10.8 Hz, 1H), 7.27 (d, J = 7.6 Hz, 1H), 6.16 (q, J = 6.4 Hz, 1H), 4.17 (d, J = 8.4 Hz, 2H), 3.82 (dd, J = 8.8 Hz, 1.6 Hz, 2H), 2.71-2.55 (m, 4H), 1.80 (d, J = 6.8 Hz, 3H), 1.76-1.66 (m, 4H), 1.44 (s, 3H) |
| Example 129 (R)-5-(5-(1-(3,5- dichloropyridin-4- yl)ethoxy)-6-fluoro- 1H-indazol-3-yl)-2-(3- ((2- hydroxyethyl)amino)- 3-methylazetidin-1- yl)nicotinonitrile | 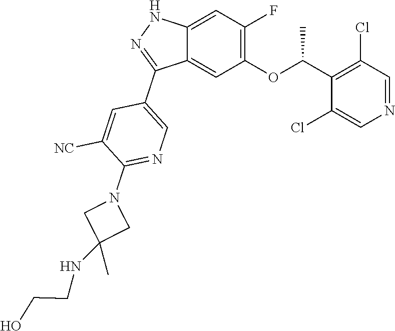 [SMILES: C[C@@H](Oc1cc2c(-c3cnc(N4CC(C)(NCCO)C4)c(C#N)c3)n[nH]c2cc1F)c1c(Cl)cncc1Cl] | LCMS m/z = 556 (M + H); 1H NMR (400 MHz, DMSO-d6) δ 13.24 (s, 1H), 8.73 (d, J = 2.4 Hz, 1 H), 8.60 (s, 2H), 8.12 (d, J = 2.8 Hz, 1H), 7.47 (d, J = 10.8 Hz, 1H), 7.23 (d, J = 7.6 Hz, 1H), 6.18 (q, J = 6.8 Hz, 1H), 4.55 (t, J = 5.6 Hz, 1H), 4.14- 4.11 (m, 2H), 4.06-4.02 (m, 2H), 3.49 (q, J = 5.6 Hz, 2H), 2.61 (t, J = 6.0 Hz, 2H), 1.79 (d, J = 6.8 Hz, 3H), 1.42 (s, 3H) |
| Example 130 1-[5-[5-[(1R)-1-(3,5- dichloro-4- pyridyl)ethoxy]-6- fluoro-1H-indazol-3- yl]-3-fluoro-2-pyridyl]- N,N,3-trimethyl- azetidin-3-amine | 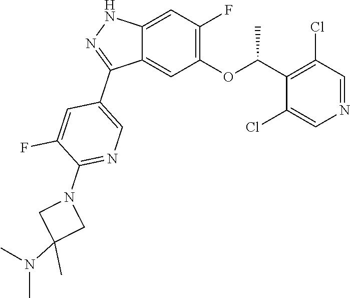 [SMILES: C[C@@H](Oc1cc2c(-c3cnc(N4CC(C)(N(C)C)C4)c(F)c3)n[nH]c2cc1F)c1c(Cl)cncc1Cl] | LCMS m/z = 533.1 (M + H); 1H NMR (400 MHz, DMSO-d6) δ = 13.12 (br s, 1H), 8.53 (s, 2H), 8.27 (t, J = 1.6 Hz, 1H), 7.61 (dd, J = 1.8, 13.2 Hz, 1H), 7.38 (d, J = 10.9 Hz, 1H), 7.21 (d, J = 7.8 Hz, 1H), 6.10 (q, J = 6.6 Hz, 1H), 3.87 (d, J = 7.6 Hz, 2H), 3.73 (dd, J = 2.0, 8.2 Hz, 2H), 2.05 (s, 6H), 1.73 (d, J = 6.6 Hz, 3H), 1.24 (s, 3H) |
| Example 132 N- (cyclopropylmethyl)-1- [5-[5-[(1R)-1-(3,5- dichloro-4- pyridyl)ethoxy]-6- fluoro-1H-indazol-3- yl]-3-fluoro-2-pyridyl]- 3-methyl-azetidin-3- amine | 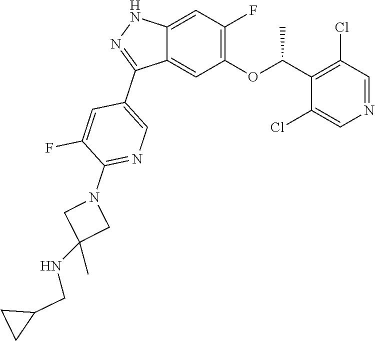 [SMILES: C[C@@H](Oc1cc2c(-c3cnc(N4CC(C)(NCC5CC5)C4)c(F)c3)n[nH]c2cc1F)c1c(Cl)cncc1Cl] | LCMS m/z = 559.3 (M + H); 1H NMR (400 MHz, DMSO-d6) δ 13.18 (s, 1H), 8.60 (s, 2H), 8.36- 8.34 (m, 1H), 7.67 (d, J = 12.8 Hz, 1H), 7.45 (d, J = 11.2 Hz, 1H), 7.28 (d, J = 7.6 Hz, 1H), 6.17 (q, J = 6.4 Hz, 1H), 4.00 (d, J = 8.0 Hz, 2H), 3.86 (d, J = 8.4 Hz, 2H), 2.41-2.40 (m, 2H), 1.80 (d, J = 6.4 Hz, 3H), 1.43 (s, 3H), 0.89-0.86 (m, 1H), 0.42-0.40 (m, 2H), 0.15-0.10 (m, 2H) |
| Example 133 5-[5-[(1R)-1-(3,5- dichloro-4- pyridyl)ethoxy]-1H- indazol-3-yl]-2-[3- [(isopropylamino)meth- yl]-3-methyl-azetidin- 1-yl]pyridine-3- carbonitrile | 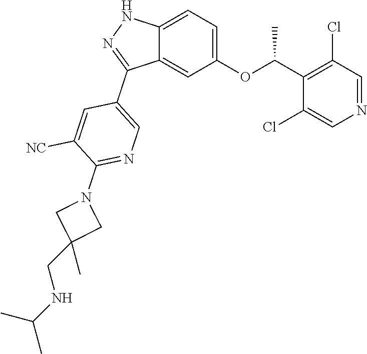 [SMILES: CC(C)NCC1(C)CN(c2ncc(-c3n[nH]c4ccc(O[C@H](C)c5c(Cl)cncc5Cl)cc34)cc2C#N)C1] | LCMS m/z = 550.1 (M + H); 1H NMR (400 MHz, DMSO-d6) δ 13.17 (s, 1H), 8.75 (d, J = 2.4 Hz, 1H), 8.58 (s, 2H), 8.12 (d, J = 2.4 Hz, 1H), 7.49 (d, J = 9.2 Hz, 1H), 7.15 (d, J = 2.4 Hz, 1H), 7.11 (dd, J = 8.8 Hz, 2.0 Hz, 1H), 6.16 (q, J = 6.0 Hz, 1H), 4.15 (d, J = 8.4 Hz, 2H), 3.99 (d, J = 8.4 Hz, 2H), 2.76- 2.65 (m, 3H), 1.77 (d, J = 6.8 Hz, 3H), 1.32 (s, 3H), 1.03 (d, J = 6.4 Hz, 6H) |
| Example 134 (R)-2-(3- ((cyclopropylmethyl)a- mino)-3- methylazetidin-1-yl)-5- (5-(1-(3,5- dichloropyridin-4- yl)ethoxy)-6-fluoro- 1H-indazol-3- yl)nicotinonitrile | 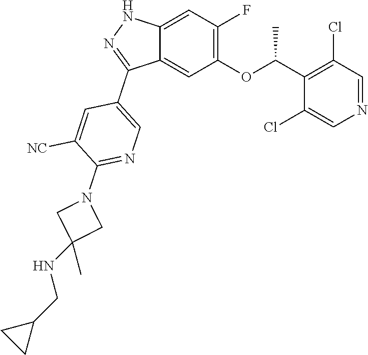 [SMILES: C[C@@H](Oc1cc2c(-c3cnc(N4CC(C)(NCC5CC5)C4)c(C#N)c3)n[nH]c2cc1F)c1c(Cl)cncc1Cl] | LCMS m/z = 566.2 (M + H); 1H NMR (400 MHz, DMSO-d6) δ 13.24 (s, 1H), 8.74-8.73 (m, 1H), 8.60 (s, 2H), 8.13 (d, J = 1.6 Hz, 1H), 7.47 (d, J = 10.8 Hz, 1H), 7.24 (d, J = 8.0 Hz, 1H), 6.18 (q, J = 6.8 Hz, 1H), 4.16-4.14 (m, 2H), 4.05- 4.03 (m, 2H), 2.43-2.42 (m, 2H), 1.80 (d, J = 6.4 Hz, 3H), 1.43 (s, 3H), 0.87-0.83 (m, 1H), 0.43-0.42 (m, 2H), 0.17-0.12 (m, 2H) |
| Example 135 (R)-5-(5-(1-(3,5- dichloropyridin-4- yl)ethoxy)-6-fluoro- 1H-indazol-3-yl)-2-(3- methyl-3-(pyrrolidin-1- yl)azetidin-1- yl)nicotinonitrile | 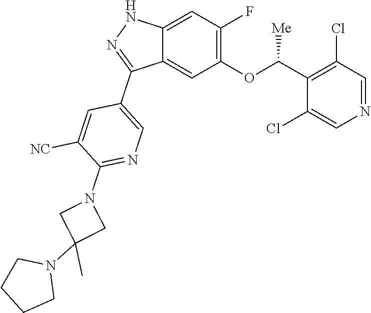 [SMILES: C[C@@H](Oc1cc2c(-c3cnc(N4CC(C)(N5CCCC5)C4)c(C#N)c3)n[nH]c2cc1F)c1c(Cl)cncc1Cl] | LCMS m/z = 566.3 (M + H); 1H- NMR (400 MHz, DMSO-d6) δ 13.25 (s, 1H), 8.73 (d, J = 2.4 Hz, 1H), 8.60 (s, 2H), 8.12 (d, J = 2.0 Hz, 1H), 7.47 (d, J = 11.2 Hz, 1H), 7.23 (d, J = 8.0 Hz, 1H), 6.17 (q, J = 6.4 Hz, 1H), 4.30 (d, J = 8.8 Hz, 2H), 3.99 (d, J = 8.8 Hz, 2H), 2.69- 2.59 (m, 4H), 1.80 (d, J = 6.8 Hz, 3H), 1.75-1.68 (m, 4H), 1.44 (s, 3H) |
| Example 136 (R)-5-(5-(1-(3,5- dichloropyridin-4- yl)ethoxy)-6-fluoro- 1H-indazol-3-yl)-2-(3′- methyl-[1,3′- biazetidin]-1′- yl)nicotinonitrile | 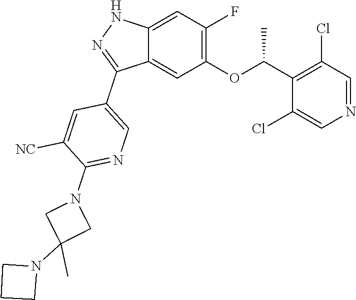 [SMILES: C[C@@H](Oc1cc2c(-c3cnc(N4CC(C)(N5CCC5)C4)c(C#N)c3)n[nH]c2cc1F)c1c(Cl)cncc1Cl] | LCMS m/z = 552.3 (M + H); 1H NMR (400 MHz, DMSO-d6) δ 13.25 (s, 1H), 8.75 (d, J = 2.4 Hz, 1H), 8.60 (s, 2H), 8.14 (d, J = 2.4 Hz, 1H), 7.47 (d, J = 10.8 Hz, 1H), 7.24 (d, J = 7.6 Hz, 1H), 6.18 (q, J = 6.4 Hz, 1H), 4.32 (d, J = 9.6 Hz, 2H), 3.94 (dd, J = 9.6 Hz, 2.4 Hz, 2H), 3.25 (t, J - 6.8 Hz, 4H), 2.00- 1.93 (m, 2H), 1.80 (d, J = 6.4 Hz, 3H), 1.29 (s, 3H) |
| Example 137 (R)-5-(5-(1-(3,5- dichloropyridin-4- yl)ethoxy)-6-fluoro- 1H-indazol-3-yl)-2-(3- methyl-3- (morpholinomethyl)aze- tidin-1- yl)nicotinonitrile | 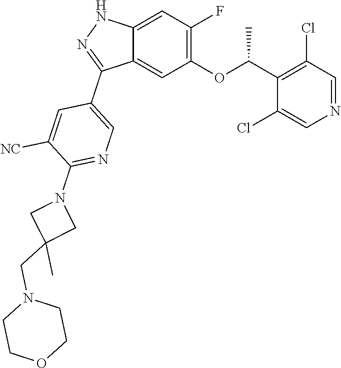 [SMILES: C[C@@H](Oc1cc2c(-c3cnc(N4CC(C)(CN5CCOCC5)C4)c(C#N)c3)n[nH]c2cc1F)c1c(Cl)cncc1Cl] | LCMS m/z = 596.3 (M + H); 1H- NMR (400 MHz, DMSO-d6) δ 13.23 (s, 1H), 8.73 (d, J = 1.6 Hz, 1H), 8.60 (s, 2H), 8.12 (d, J = 1.6 Hz, 1H), 7.47 (d, J = 10.8 Hz, 1H), 7.24 (d, J = 7.6 Hz, 1H), 6.18 (q, J = 6.8 Hz, 1H), 4.08 (d, J = 8.8 Hz, 2H), 3.99 (d, J = 8.4 Hz, 2H), 3.61- 3.55 (m, 4H), 2.54 (s, 2H), 2.41- 2.33 (m, 4H), 1.80 (d, J = 6.8 Hz, 3H), 1.39 (s, 3H) |

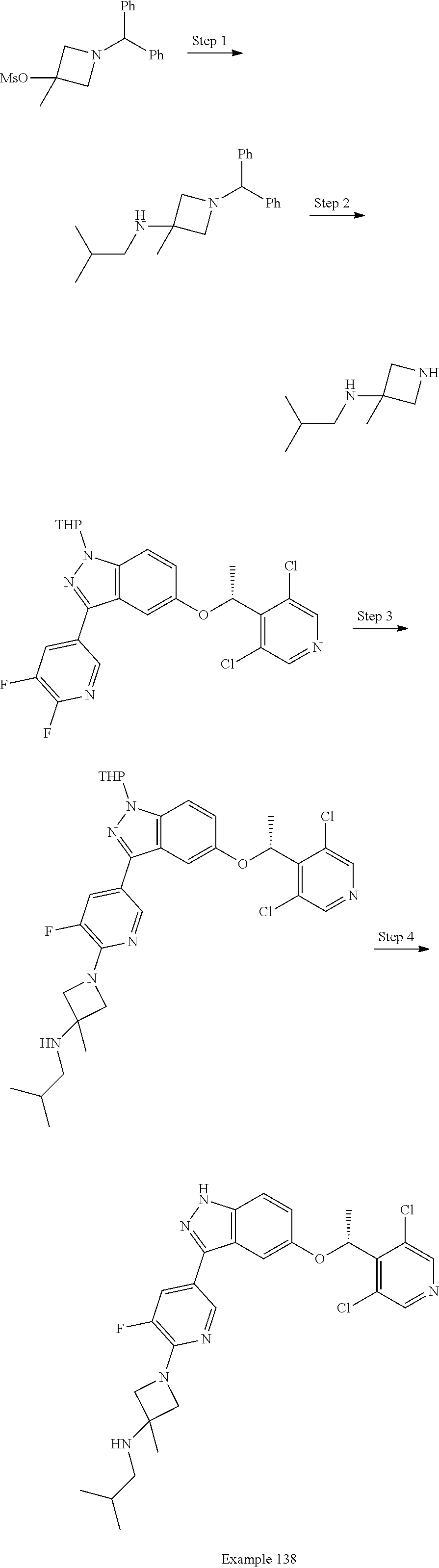

### Example 138. 1-[5-[5-[(1R)-1-(3,5-dichloro-4-pyridyl)ethoxy]-1H-indazol-3-yl]-3-fluoro-2-pyridyl]-N-isobutyl-3-methyl-azetidin-3-amine

Step 1. 1-Benzhydryl-N-isobutyl-3-methyl-azetidin-3-amine. To a solution of (1-benzhydryl-3-methyl-azetidin-3-yl) methanesulfonate (12.0 g, 36.21 mmol, 1.0 eq) and 2-methylpropan-1-amine (7.9 g, 72.41 mmol, 2.0 eq) in CH₃CN (240 mL) was added Cs₂CO₃ (35.3 g, 108.6 mmol, 3.0 eq). The reaction mixture was stirred for 3 h at 80° C. After the reaction was completed, the mixture was cooled down to rt and concentrated in vacuo. The crude product was purified by silica gel flash column chromatography (Petroleum Ether/EtOAc=1/1) to afford a colorless oil 7.0 g, 63%). LCMS m/z=309.3 (M+1).

Step 2. N-Isobutyl-3-methyl-azetidin-3-amine. To a solution of 1-benzhydryl-N-isobutyl-3-methyl-azetidin-3-amine (7.0 g, 22.7 mmol, 1.0 eq) in MeOH (70 mL) was added Pd/C (6.9 g, 10% wt) in portions under N₂ protected. The suspension was degassed under vacuum and charged with hydrogen three times. The mixture was then stirred for 16 h at rt under H₂ balloon. The solid was filtered out and the filtrate was concentrated to give a colorless oil. LCMS m/z=143.3 (M+1).

Step 3. 1-[5-[5-[(1R)-1-(3,5-Dichloro-4-pyridyl)ethoxy]-1-tetrahydropyran-2-yl-indazol-3-yl]-3-fluoro-2-pyridyl]-N-isobutyl-3-methyl-azetidin-3-amine. To a solution of 5-[(1R)-1-(3,5-dichloro-4-pyridyl)ethoxy]-3-(5,6-difluoro-3-pyridyl)-1-tetrahydropyran-2-yl-indazole (3.0 g, 5.94 mmol, 1.0 eq) and N-isobutyl-3-methyl-azetidin-3-amine (844 mg, 5.94 mmol, 1.0 eq) in DMSO (30 mL) was added DIEA (4.6 g, 35.62 mmol, 6.0 eq). The reaction mixture was stirred for 16 h at 105° C. After the reaction was completed, the mixture was cooled down to rt, diluted with EtOAc (50 mL) and washed with brine (50 mL×2). The organic layer was dried over Na₂SO₄ and concentrated. The crude product was purified by silica gel flash column chromatography (Petroleum Ether/EtOAc=1/1) to give a white solid (2.2 g, 59%). LCMS m/z=627.2 (M+1).

Step 4. 1-[5-[5-[(1R)-1-(3,5-dichloro-4-pyridyl)ethoxy]-1H-indazol-3-yl]-3-fluoro-2-pyridyl]-N-isobutyl-3-methyl-azetidin-3-amine. To a solution of 1-[5-[5-[(1R)-1-(3,5-dichloro-4-pyridyl)ethoxy]-1-tetrahydropyran-2-yl-indazol-3-yl]-3-fluoro-2-pyridyl]-N-isobutyl-3-methyl-azetidin-3-amine (2.0 g, 3.19 mmol, 1.0 eq) in DCM (20 mL) was added HCl/EA (10 mL). The reaction mixture was stirred for 3 h at rt. After the reaction was completed, the reaction mixture was concentrated. The solution was treated with NaHCO₃ aqueous solution to adjust the pH to 7-8, then was extracted with DCM (40 mL×3). The combined organic layers were washed with brine, dried over Na₂SO₄, and concentrated in vacuum. The crude product was purified by silica gel flash column chromatography (DCM/MeOH=20/1) to give a white solid (1.09 g, 63%). LCMS m/z=543.3 (M+1); 1H NMR (400 MHz, DMSO-d6) δ13.11 (s, 1H), 8.57 (s, 2H), 8.37 (d, J=1.6 Hz, 1H), 7.69 (d, J=13.2 Hz, 1H), 7.47 (d, J=8.8 Hz, 1H), 7.20 (d, J=1.6 Hz, 1H), 7.10 (dd, J=8.8 Hz, 2.4 Hz, 1H), 6.14 (q, J=6.8 Hz, 1H), 4.00 (d, J=7.2 Hz, 2H), 3.87 (d, J=7.2 Hz, 2H), 2.35-2.33 (m, 2H), 1.76 (d, J=6.8 Hz, 3H), 1.63-1.58 (m, 1H), 1.44 (s, 3H), 0.90 (d, J=6.8 Hz, 6H).

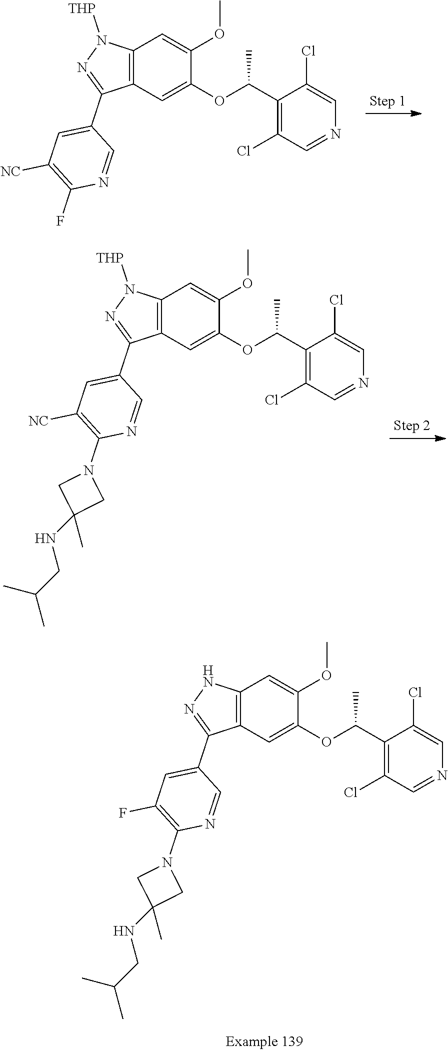

### Example 139. (R)-5-(5-(1-(3,5-dichloropyridin-4-yl)ethoxy)-6-methoxy-1H-indazol-3-yl)-2-(3-(isobutylamino)-3-methylazetidin-1-yl)nicotinonitrile

Step 1. 5-[5-[(1R)-1-(3,5-Dichloro-4-pyridyl)ethoxy]-6-methoxy-1-tetrahydropyran-2-yl-indazol-3-yl]-2-[3-(isobutylamino)-3-methyl-azetidin-1-yl]pyridine-3-carbonitrile. To a solution of 5-[5-[(1R)-1-(3,5-dichloro-4-pyridyl)ethoxy]-6-methoxy-1-tetrahydropyran-2-yl-indazol-3-yl]-2-fluoro-pyridine-3-carbonitrile (2.5 g, 4.61 mmol, 1.0 eq) and N-isobutyl-3-methyl-azetidin-3-amine (656 mg, 4.61 mmol, 1.0 eq) in DMSO (60 mL) was added DIEA (3.6 g, 27.66 mmol, 6.0 eq). The reaction mixture was stirred at 60° C. for 16 h. After the reaction was completed, EtOAc (200 mL) was added to the mixture, which washed with brine (100 mL×2). The organic layer was dried over Na₂SO₄ and concentrated in vacuum. The crude product was purified by silica gel column (DCM/MeOH=20/1) to give a white solid (2.0 g, 67%). LCMS m/z=664.3 (M+1).

Step 2. (R)-5-(5-(1-(3,5-dichloropyridin-4-yl)ethoxy)-6-methoxy-1H-indazol-3-yl)-2-(3-(isobutylamino)-3-methylazetidin-1-yl)nicotinonitrile. To a solution of 5-[5-[(1R)-1-(3,5-dichloro-4-pyridyl)ethoxy]-6-methoxy-1-tetrahydropyran-2-yl-indazol-3-yl]-2-[3-(isobutylamino)-3-methyl-azetidin-1-yl]pyridine-3-carbonitrile (2.0 g, 3.01 mmol, 1.0 eq) in EA (30 mL) was added HCl/EA (30 mL) at 0° C. The resulting mixture was stirred at rt for 5 h. After the reaction was completed, the reaction mixture was diluted with the saturated NaHCO₃ solution to adjust pH to 7-8. The aqueous layer was extracted with EtOAc (70 mL×2) and the combined organic layers were dried over Na₂SO₄ and then concentrated in vacuum. The crude product was purified by silica gel column (DCM/MeOH=15/1) to afford an off-white solid (1.12 g, 63%). LCMS m/z=580.5 (M+1); 1H NMR (400 MHz, DMSO-d6) δ 12.97 (s, 1H), 8.71 (d, J=2.8 Hz, 1H), 8.58 (s, 2H), 8.03 (d, J=2.0 Hz, 1H), 7.01 (s, 1H), 7.00 (s, 1H), 5.98 (q, J=6.8 Hz, 1H), 4.10 (d, J=8.4 Hz, 2H), 4.01 (d, J=8.4 Hz, 2H), 3.86 (s, 3H), 2.32 (d, J=8.4 Hz, 2H), 2.25 (brs, 1H), 1.75 (d, J=6.8 Hz, 3H), 1.61-1.58 (m, 1H), 1.41 (s, 3H), 0.90 (d, J=6.8 Hz, 6H).

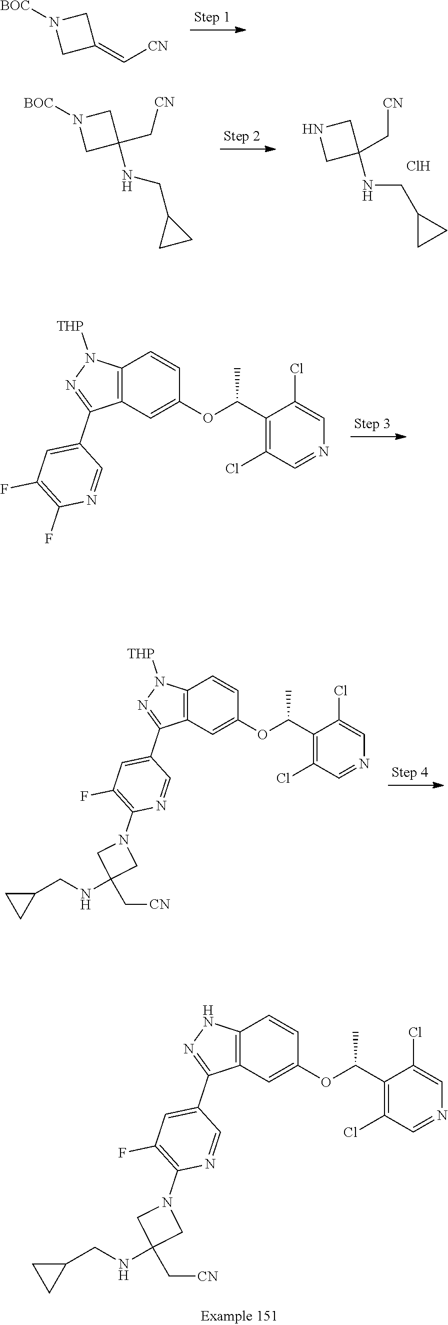

### Example 151. 2-[3-(Cyclopropylmethyl-amino)-1-[5-[5-[(1R)-1-(3,5-dichloro-4-pyridyl)ethoxy]-1H-indazol-3-yl]-3-fluoro-2-pyridyl]azetidin-3-yl]acetonitrile

Step 1. tert-Butyl 3-(cyanomethyl)-3-(cyclopropylmethylamino)azetidine-1-carboxylate. To a solution of tert-butyl 3-(cyanomethylene)azetidine-1-carboxylate (1.0 g, 5.15 mmol, 1.0 eq) and cyclopropylmethanamine (732 mg, 10.30 mmol, 2.0 eq) in CH3CN (10 mL) was added DBU (2.35 g, 15.45 mmol, 3.0 eq). The reaction mixture was stirred for 16 h at 70° C. After the reaction was completed, the mixture was cooled to rt and concentrated in vacuum. The crude product was purified by silica gel flash column chromatography (Petroleum Ether/EtOAc=1/1) to afford a yellow solid (1.2 g, 88% yield). LCMS m/z=266.3 (M+1); 1HNMR (400 MHz, DMSO-d6) δ 3.70-3.69 (m, 2H), 3.62-3.60 (m, 2H), 2.94 (s, 2H), 2.51-2.49 (m, 2H), 1.37 (s, 9H), 0.84-0.80 (m, 1H), 0.40-0.38 (m, 2H), 0.13-0.09 (m, 2H).

Step 2. 2-[3-(Cyclopropylmethylamino)azetidin-3-yl]acetonitrile hydrochloride. To a solution of tert-Butyl 3-(cyanomethyl)-3-(cyclopropylmethylamino)azetidine-1-carboxylate (700 mg, 2.64 mmol, 1.0 eq) in DCM (10 mL) was added HCl/dixoane (4 M, 3 mL). The reaction solution was stirred for 4 h at rt. After the reaction was completed, the reaction mixture was concentrated to give a light-yellow solid (400 mg, 75% yield). LCMS m/z=166.2 (M+1).

Step 3. 2-[3-(Cyclopropylmethylamino)-1-[5-[5-[(1R)-1-(3,5-dichloro-4-pyridyl)ethoxy]-1-tetrahydropyran-2-yl-indazol-3-yl]-3-fluoro-2-pyridyl]azetidin-3-yl]acetonitrile. To a solution of 5-[(1R)-1-(3,5-dichloro-4-pyridyl)ethoxy]-3-(5,6-difluoro-3-pyridyl)-1-tetrahydropyran-2-yl-indazole (140 mg, 0.28 mmol, 1.0 eq) and 2-[3-(cyclopropylmethylamino)azetidin-3-yl]acetonitrile hydrochloride (69 mg, 0.42 mmol, 1.5 eq) in DMSO (5 mL) was added DIEA (107 mg, 0.83 mmol, 3.0 eq). The reaction mixture was stirred at 100° C. for 8 h. After the reaction was completed, it diluted with EtOAc (50 mL), and was washed with brine (30 mL×2). The organic layer was dried over Na2SO4 and concentrated in vacuum. The crude product was purified by Prep-TLC (DCM/MeOH=30/1) to afford a white solid (140 mg, 78% yield). LCMS m/z=650.3 (M+1).

Step 4. 2-[3-(Cyclopropylmethyl-amino)-1-[5-[5-[(1R)-1-(3,5-dichloro-4-pyridyl)ethoxy]-1H-indazol-3-yl]-3-fluoro-2-pyridyl]azetidin-3-yl]acetonitrile To a solution of 2-[3-(cyclopropylmethylamino)-1-[5-[5-[(1R)-1-(3,5-dichloro-4-pyridyl)ethoxy]-1-tetrahydropyran-2-yl-indazol-3-yl]-3-fluoro-2-pyridyl]azetidin-3-yl]acetonitrile (140 mg, 0.22 mmol, 1.0 eq) in EtOAc (5 mL) was added HCl/EtOAc (4 M, 5 mL) at 0° C. The resulting mixture was stirred at rt for 16 h. After the reaction was completed, the reaction mixture was added a saturated NaHCO₃ solution to adjust the pH=7˜8, and then extracted with EtOAc (30 mL×2). The combined organic layers were dried over Na2SO4 and concentrated. The crude product was purified by Prep-TLC (DCM/MeOH=15/1) to give a white solid (50 mg, 41% yield). LCMS m/z=566.3 (M+1); 1H NMR (400 MHz, DMSO-d6) δ 13.13 (s, 1H), 8.57 (s, 2H), 8.39-8.37 (m, 1H), 7.73 (dd, J=13.2 Hz, 2.0 Hz, 1H), 7.48 (d, J=9.2 Hz, 1H), 7.20 (d, J=1.6 Hz, 1H), 7.10 (dd, J=8.8 Hz, 2.4 Hz, 1H), 6.15 (q, J=6.4 Hz, 1H), 4.11-4.06 (m, 2H), 3.99-3.95 (m, 2H), 3.13 (s, 2H), 2.43 (d, J=6.0 Hz, 2H), 1.76 (d, J=6.4 Hz, 3H), 0.91-0.83 (m, 1H), 0.45-0.41 (m, 2H), 0.18-0.14 (m, 2H).

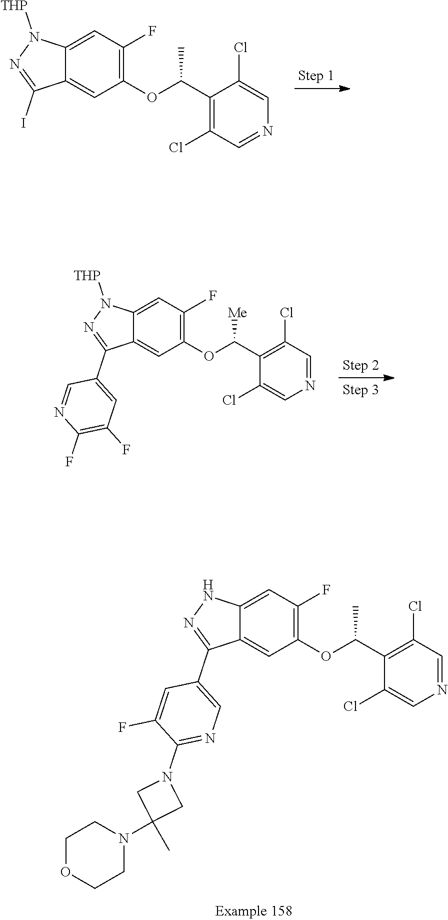

### Example 158

Step 1. 5-((R)-1-(3,5-Dichloropyridin-4-yl)ethoxy)-3-(5,6-difluoropyridin-3-yl)-6-fluoro-1-(tetrahydro-2H-pyran-2-yl)-1H-indazole. A mixture of 5-[(1R)-1-(3,5-dichloro-4-pyridyl)ethoxy]-6-fluoro-3-iodo-1-tetrahydropyran-2-yl-indazole (2 g, 3.73 mmol, 1.00 equiv), 2,3-difluoro-5-(4,4,5,5-tetramethyl-1,3,2-dioxaborolan-2-yl)pyridine (1.1 g, 4.48 mmol, 1.2 equiv), [1,1′-bis(diphenylphosphino)ferrocene]dichloropalladium(II) (273 mg, 0.373 mmol, 0.1 equiv), potassium carbonate (1.03 g, 7.46 mmol, 2.0 equiv), water (4 mL) and 1,4-dioxane (40 mL) was sparged with nitrogen for 10 minutes then heated at 95° C. for 16 h. The reaction was cooled to room temperature and filtered through Celite (5 g) and washed with ethyl acetate (100 mL). The filtrate was dried over sodium sulfate, filtered, and concentrated under reduced pressure. The residue was purified on a Biotage automated chromatography system (Sorbtech 80 g silica gel column), eluting with a gradient of 0 to 50% ethyl acetate in hexanes to give an off-white solid (1.88 g, 96%). LCMS m/z=523.0 (M+H).

Step 2. 4-(1-(5-(5-((R)-1-(3,5-Dichloropyridin-4-yl)ethoxy)-6-fluoro-1-(tetrahydro-2H-pyran-2-yl)-1H-indazol-3-yl)-3-fluoropyridin-2-yl)-3-methylazetidin-3-yl)morpholine. A mixture of 5-((R)-1-(3,5-dichloropyridin-4-yl)ethoxy)-3-(5,6-difluoropyridin-3-yl)-6-fluoro-1-(tetrahydro-2H-pyran-2-yl)-1H-indazole (300 mg, 0.573 mmol, 1 equiv), 4-(3-methylazetidin-3-yl)morpholine (264 mg, 1.15 mmol, 2 equiv) and potassium carbonate (475 mg, 5.44 mmol, 6 equiv) in acetonitrile (10 mL) was heated at 80° C. for 24 hours. The mixture was cooled to room temperature and diluted with water (50 mL). The precipitate was stirred at room temperature for 1.5 hours, filtered, washed with water (20 mL) and dried at 40° C. under vacuum for 20 hours to give a white solid (350 mg, 93%), which was used directly in the next step. LCMS m/z=659.1 (M+H).

Step 3. (R)-4-(1-(5-(5-(1-(3,5-Dichloropyridin-4-yl)ethoxy)-6-fluoro-1H-indazol-3-yl)-3-fluoropyridin-2-yl)-3-methylazetidin-3-yl)morpholine. 4-(1-(5-(5-((R)-1-(3,5-Dichloropyridin-4-yl)ethoxy)-6-fluoro-1-(tetrahydro-2H-pyran-2-yl)-1H-indazol-3-yl)-3-fluoropyridin-2-yl)-3-methylazetidin-3-yl)morpholine (350 mg, 0.53 mmol, 1.0 equiv) was treated with a 1 to 1 mixture of dichloromethane and trifluoroacetic acid (4 mL) at room temperature for 5 hours. The mixture was concentrated under reduced pressure. The residue was dissolved in ethyl acetate (15 mL) and washed with saturated sodium carbonate (10 mL). The organic layer was concentrated under reduced pressure. The residue was redissolved in dimethyl sulfoxide (10 mL) and purified on a Biotage automated chromatography system (RediSep Rf GOLD 100 g HP C18 column), eluting with a gradient of 0 to 100% acetonitrile in water to give an off-white solid after lyophilization (204 mg, 67% yield). LCMS m/z=575.1 (M+H); 1H NMR (400 MHz, DMSO-d6) δ=13.19 (br s, 1H), 8.60 (s, 2H), 8.34 (t, J=1.6 Hz, 1H), 7.69 (dd, J=1.8, 13.1 Hz, 1H), 7.45 (d, J=10.9 Hz, 1H), 7.27 (d, J=7.9 Hz, 1H), 6.17 (q, J=6.6 Hz, 1H), 3.98 (d, J=7.8 Hz, 2H), 3.81 (dd, J=2.0, 8.2 Hz, 2H), 3.66-3.54 (m, 4H), 2.47-2.36 (m, 4H), 1.80 (d, J=6.7 Hz, 3H), 1.35 (s, 3H).

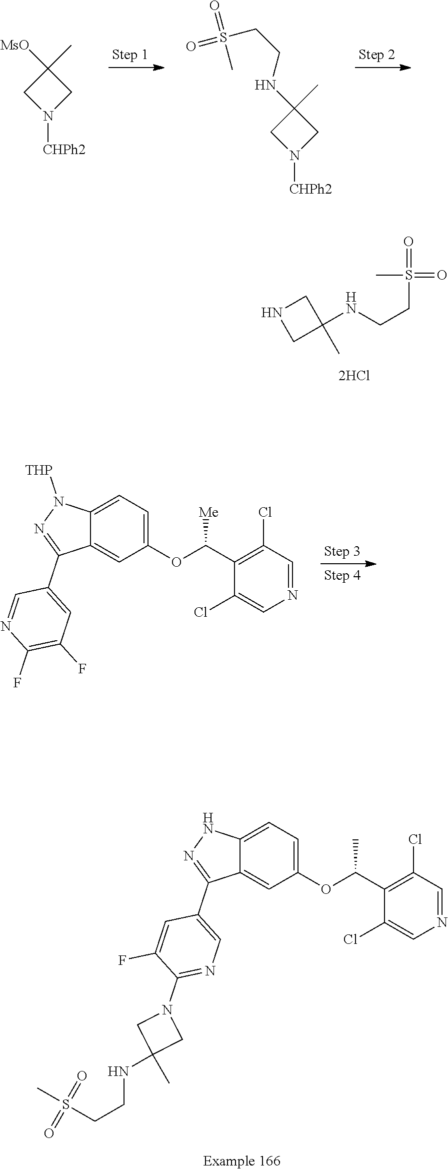

### Example 166

Step 1. 1-Benzhydryl-3-methyl-N-(2-(methylsulfonyl)ethyl)azetidin-3-amine. (1-Benzhydryl-3-methyl-azetidin-3-yl) methanesulfonate (1.35 g, 4.06 mmol, 1 equiv) was treated with 2-methylsulfonylethanamine (1.0 g, 8.12 mmol, 2.0 equiv) and N,N-diisopropylethylamine (2.0 mL, 12.18 mmol, 3.0 equiv) in 1,4-dioxane (14 mL) at 100° C. for 24 hours. After cooling to room temperature, the reaction was diluted with water (20 mL) and extracted with ethyl acetate (2×20 mL). The combined organic layers were dried over sodium sulfate, filtered, and concentrated under reduced pressure onto silica gel (9 g). The residue was purified on a Biotage automated chromatography system (Sorbtech 40 g silica gel column), eluting with a gradient of 0 to 5% methanol in dichloromethane to give compound a yellow oil (0.96 g, 66%). LCMS m/z=359.1 (M+H).

Step 2. 3-Methyl-N-(2-(methylsulfonyl)ethyl)azetidin-3-amine dihydrochloride. 1-Benzhydryl-3-methyl-N-(2-(methylsulfonyl)ethyl)azetidin-3-amine (0.96 g, 2.68 mmol, 1 equiv), concentrated HCl (0.8 mL, 9.6 mmol, 3.6 equiv) and 20% palladium hydroxide on carbon (50% wet, 0.18 g, 0.13 mmol, 0.05 equiv) in ethanol (50 mL) was hydrogenated at 50 psi overnight. The mixture was filtered through Celite, and was washed with a 19 to 1 mixture of ethanol and water (200 mL). The filtrate was concentrated under reduced pressure to give a residue, which was then rinsed with hexanes (2×10 mL). The residue was dried under vacuum at 40° C. overnight to give a tan solid (0.70 g, 98%). LCMS m/z=193.1 (M+H) free base.

Step 3. 1-(5-(5-((R)-1-(3,5-Dichloropyridin-4-yl)ethoxy)-1-(tetrahydro-2H-pyran-2-yl)-1H-indazol-3-yl)-3-fluoropyridin-2-yl)-3-methyl-N-(2-(methylsulfonyl)ethyl)azetidin-3-amine. 3-Methyl-N-(2-(methylsulfonyl)ethyl)-azetidin-3-amine dihydrochloride (157 mg, 0.6 mmol, 2 equiv) and potassium carbonate (246 mg, 1.8 mmol, 6 equiv) were added to a solution of 5-[(1R)-1-(3,5-dichloro-4-pyridyl)ethoxy]-3-(5,6-difluoro-3-pyridyl)-1-tetrahydropyran-2-yl-indazole (150 mg, 0.3 mmol, 1 equiv) in acetonitrile (10 mL). After stirring at 80° C. for 16 hours, the reaction was cooled to room temperature and diluted with ethyl acetate (20 mL) and water (10 mL). The layers were separated, and the aqueous layer was extracted with ethyl acetate (2×10 mL). The combined organic layer was washed with saturated brine (20 mL), dried over sodium sulfate, filtered, and concentrated under reduced pressure. The residue was purified on a Biotage automated chromatography system (Sorbtech 25 g silica gel column), eluting with a gradient of 0 to 100% ethyl acetate in hexanes to give an off-white solid (180 mg, 90%). LCMS m/z=677.2 (M+H).

Step 4. (R)-1-(5-(5-(1-(3,5-Dichloropyridin-4-yl)ethoxy)-1H-indazol-3-yl)-3-fluoropyridin-2-yl)-3-methyl-N-(2-(methylsulfonyl)ethyl)azetidin-3-amine. Trifluoroacetic acid (2 mL) was added to a solution of 1-(5-(5-((R)-1-(3,5-dichloropyridin-4-yl)ethoxy)-1-(tetrahydro-2H-pyran-2-yl)-1H-indazol-3-yl)-3-fluoropyridin-2-yl)-3-methyl-N-(2-(methylsulfonyl)ethyl)azetidin-3-amine (180 mg, 0.27 mmol, 1.0 equiv) in dichloromethane (2 mL) at room temperature. After stirring at room temperature for 4 hours, the volatiles were removed under reduced pressure. The residue was diluted with ethyl acetate (20 mL) and water (10 mL), and adjusted to pH 9 with saturated sodium carbonate. The layers were separated, and the aqueous layer was extracted with ethyl acetate (3×10 mL). The combined organic layers were washed with saturated brine (20 mL), dried over sodium sulfate, filtered, and concentrated under reduced pressure. The residue was redissolved in dimethyl sulfoxide (10 mL) and purified on a Biotage automated chromatography system (RediSep Rf GOLD 100 g HP C18 column), eluting with a gradient of 0 to 100% acetonitrile in water to give an off-white solid after lyophilization (89 mg, 56%). LCMS m/z=593 (M+H); 1H NMR (400 MHz, DMSO-d6) δ=13.03 (s, 1H), 8.50 (s, 2H), 8.30 (t, J=1.5 Hz, 1H), 7.63 (dd, J=1.7, 13.2 Hz, 1H), 7.40 (d, J=9.0 Hz, 1H), 7.13 (d, J=2.0 Hz, 1H), 7.03 (dd, J=2.3, 9.0 Hz, 1H), 6.07 (q, J=6.7 Hz, 1H), 3.93 (d, J=7.8 Hz, 2H), 3.83 (dd, J=2.0, 8.3 Hz, 2H), 3.15 (t, J=6.7 Hz, 2H), 2.97 (s, 3H), 2.92-2.87 (m, 2H), 2.51 (br s, 1H), 1.69 (d, J=6.6 Hz, 3H), 1.38 (s, 3H).

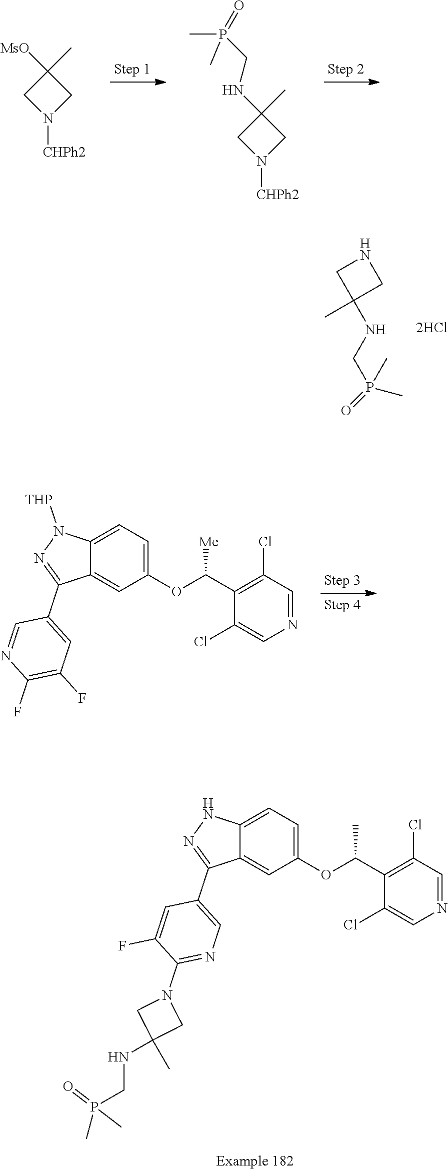

### Example 182

Step 1. (((1-Benzhydryl-3-methylazetidin-3-yl)amino)methyl)dimethylphosphine oxide. Macroporous carbonate resin (2.7 mmol/g, 6.6 g, 17.8 mmol, 3 equiv) was added to a suspension of dimethylphosphorylmethanamine HCl (0.85 g, 5.9 mmol) in acetonitrile (60 mL). The mixture was stirred overnight then filtered, rinsing the resin with acetonitrile (20 mL). The filtrate was then treated with 1-benzhydryl-3-methyl-azetidin-3-yl methanesulfonate (2.1 g, 6.3 mmol, 1.07 equiv) and N,N-diisopropylethylamine (5.2 mL, 29.8 mmol, 5 equiv) and heated at 70° C. for 6 hours. The mixture was cooled to room temperature, diluted with water (50 mL) and extracted with ethyl acetate (2×50 mL). The combined organic layers were dried over sodium sulfate, filtered, and concentrated under reduced pressure onto silica gel (12 g). The residue was purified on a Biotage automated chromatography system (Sorbtech 80 g silica gel column), eluting with a gradient of 0 to 10% methanol in dichloromethane to give (1.03 g, 51%) as a yellow oil. LCMS m/z=343 (M+H).

Step 2. Dimethyl(((3-methylazetidin-3-yl)amino)methyl)phosphine oxide bis(trifluoroacetic acid) salt (667-3): (((1-Benzhydryl-3-methylazetidin-3-yl)amino)methyl)dimethylphosphine oxide (1.03 g, 3.0 mmol), 20% palladium(II) hydroxide on carbon (50% wet, 0.46 g, 0.32 mmol, 0.1 equiv) and trifluoroacetic acid (1.8 mL, 15.7 mmol, 5.2 equiv) in ethanol (100 mL) was hydrogenated at 50 psi for 16 hours. The mixture was filtered through Celite, that was washed with a 9 to 1 mixture of ethanol and water (100 mL). The filtrate was concentrated under reduced pressure and the residue washed with hexanes (2×10 mL). The residue was dried under vacuum at 40° C. overnight to give a yellow wax (1.82 g, >100%).

Step 3. (((1-(5-(5-((R)-1-(3,5-Dichloropyridin-4-yl)ethoxy)-1-(tetrahydro-2H-pyran-2-yl)-1H-indazol-3-yl)-3-fluoropyridin-2-yl)-3-methylazetidin-3-yl)amino)methyl)dimethylphosphine oxide. A mixture of 5-[(1R)-1-(3,5-dichloro-4-pyridyl)ethoxy]-3-(5,6-difluoro-3-pyridyl)-1-tetrahydropyran-2-yl-indazole (100 mg, 0.20 mmol), product step 2 (170 mg, 0.42 mmol, 2 equiv) and potassium carbonate (140 mg, 1.0 mmol, 5 equiv) in acetonitrile (15 mL) was heated at 80° C. for 8 hours. The mixture was cooled to room temperature, diluted with water (50 mL) then extracted with ethyl acetate (2×50 mL). The combined organic layers were dried over sodium sulfate, filtered, and concentrated under reduced pressure onto silica gel (2 g). The residue was purified on a Biotage automated chromatography system (Sorbtech 12 g silica gel column), eluting with a gradient of 0 to 10% methanol in dichloromethane to give a colorless oil after drying under vacuum at 40° C. overnight (90 mg, 68% yield). LCMS m/z=661 (M+H).

Step 4. (R)-(((1-(5-(5-(1-(3,5-Dichloropyridin-4-yl)ethoxy)-1H-indazol-3-yl)-3-fluoropyridin-2-yl)-3-methylazetidin-3-yl)amino)methyl)dimethylphosphine oxide. (((1-(5-(5-((R)-1-(3,5-dichloropyridin-4-yl)ethoxy)-1-(tetrahydro-2H-pyran-2-yl)-1H-indazol-3-yl)-3-fluoropyridin-2-yl)-3-methylazetidin-3-yl)amino)methyl)dimethylphosphine oxide (90 mg, 0.13 mmol) was treated with a 1 to 1 mixture of dichloromethane and trifluoroacetic acid (4 mL) overnight. The mixture was concentrated under reduced pressure then dissolved in dichloromethane (10 mL) and concentrated under reduced pressure. The residue was dissolved in dichloromethane (20 mL) and a 50% potassium carbonate solution (10 mL). The layers were separated, and the aqueous layer was extracted with dichloromethane (10 mL). The combined organic layers were concentrated under reduced pressure onto Celite (6 g). The residue was purified on a Biotage automated chromatography system (Redisep C18 100 g column), eluting with a gradient of 0 to 10% acetonitrile in water. Product containing fractions were lyophilized to give a white solid (72 mg, 91%). LCMS m/z=577 (M+H); 1H NMR (400 MHz, DMSO-d6) δ=13.04 (br s, 1H), 8.50 (s, 2H), 8.30 (t, J=1.5 Hz, 1H), 7.62 (dd, J=1.8, 13.2 Hz, 1H), 7.40 (d, J=9.0 Hz, 1H), 7.13 (d, J=2.0 Hz, 1H), 7.03 (dd, J=2.3, 9.0 Hz, 1H), 6.07 (q, J=6.6 Hz, 1H), 3.97 (d, J=7.9 Hz, 2H), 3.80 (dd, J=1.8, 8.3 Hz, 2H), 2.78 (t, J=8.8 Hz, 2H), 2.42-2.38 (m, 1H), 1.69 (d, J=6.6 Hz, 3H), 1.38 (s, 3H), 1.33 (d, J=13.0 Hz, 6H).

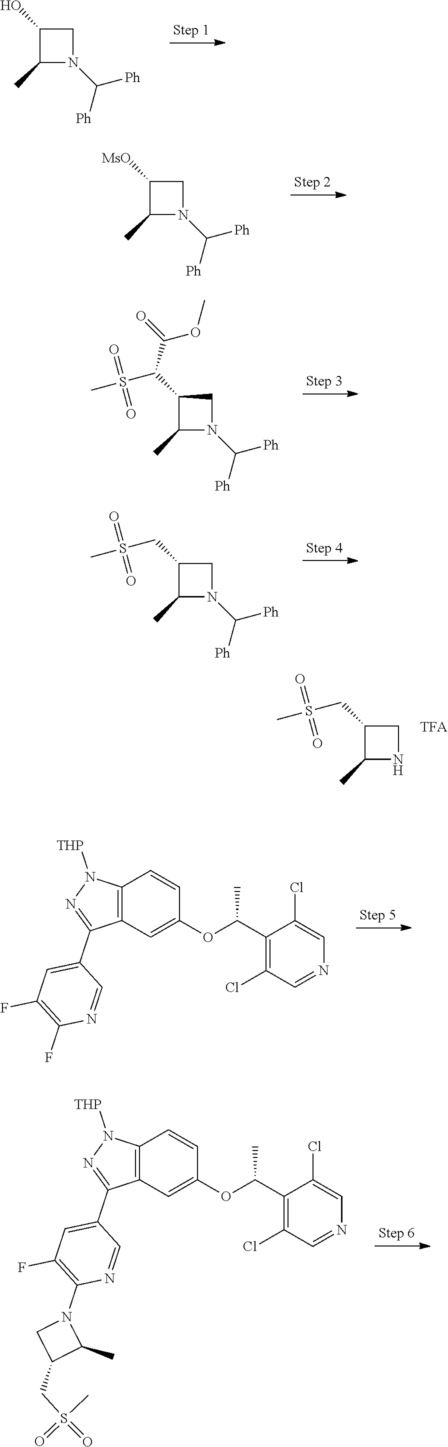

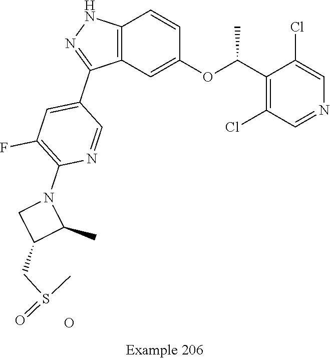

### Example 206. 5-((R)-1-(3,5-dichloropyridin-4-yl)ethoxy)-3-(5-fluoro-6-((2S,3R)-2-methyl-3-((methylsulfonyl)methyl)azetidin-1-yl)pyridin-3-yl)-1H-indazole

Step 1. [(2S,3R)-1-benzhydryl-2-methyl-azetidin-3-yl]methanesulfonate. To a solution of (2S,3R)-1-benzhydryl-2-methyl-azetidin-3-ol (14.0 g, 55.26 mmol, 1.0 eq) and TEA (11.18 g, 110.52 mmol, 2.0 eq) in DCM (150 mL) was added MsCl (8.55 g, 74.60 mmol, 1.35 eq) dropwise at 0° C. The resulting mixture was gradually warmed up to rt with stirring for 3 h. The mixture was then poured into H₂O (100 mL) and extracted with DCM (100 mL×2). The combined organic layers were washed with brine (150 mL), dried over Na₂SO₄ and concentrated. The crude product was purified by silica gel column (Petroleum ether/Ethyl acetate=5/1) to give a white solid (15.0 g, 82% yield). LCMS m/z=332.2 (M+1).

Step 2. methyl (2S)-2-[(2S)-1-benzhydryl-2-methyl-azetidin-3-yl]-2-methylsulfonyl-acetate. To a solution of methyl 2-methylsulfonylacetate (6.43 g, 42.24 mmol, 1.4 eq) in DMF (150 mL) was added NaH (1.93 g, 48.28 mmol, 60% in mineral oil, 1.6 eq) at 0° C., and then stirred for 2 h at rt. [(2S,3R)-1-benzhydryl-2-methyl-azetidin-3-yl]methanesulfonate. To a solution of (2S,3R)-1-benzhydryl-2-methyl-azetidin-3-ol (10.00 g, 30.17 mmol, 1.0 eq) in DMF (150 mL) was added, and the reaction was stirred for 7 h at 80° C. After the reaction was completed, the mixture was quenched with H₂O (500 mL), and was extracted with ethyl acetate (500 mL×2). The combined organic layers were washed with brine (800 mL×2), dried over Na₂SO₄, and concentrated. The crude product was purified by silica gel column (Petroleum ether/Ethyl acetate=4/1) to give a white solid (11.0 g, 94% yield). LCMS m/z=388.3 (M+1).

Step 3. (2S,3R)-1-benzhydryl-2-methyl-3-(methylsulfonylmethyl)azetidine. To a solution of methyl (2S)-2-[(2S)-1-benzhydryl-2-methyl-azetidin-3-yl]-2-methylsulfonyl-acetate. To a solution of methyl 2-methylsulfonylacetate (5.00 g, 12.90 mmol, 1.0 eq) in DMA (500 mL) was added LiCl (4.38 g, 103.23 mmol, 8.0 eq) at rt. The mixture was stirred at 150° C. for 3 h, and then was poured into H₂O (1000 mL), and extracted with ethyl acetate (1000 mL×2). The combined organic layers were washed with brine (1000 mL×3), dried over Na₂SO₄ and concentrated. The crude product was purified by silica gel column (Petroleum ether/Ethyl acetate=2/1) to give a white solid (3.8 g, 89% yield). LCMS m/z=330.2 (M+1).

Step 4. (2S,3R)-2-methyl-3-(methylsulfonylmethyl)azetidine TFA salt. To a solution of (2S,3R)-1-benzhydryl-2-methyl-3-(methylsulfonylmethyl)azetidine (2.30 g, 6.98 mmol, 1.0 eq) in MeOH (60 mL) and TFA (4 mL) was added Pd(OH)₂ (345 mg, 15% w.t.) under N₂ protection. The suspension was degassed under vacuum and purged with H₂ three times. The mixture was stirred under H₂ balloon at rt for 16 h, was filtered and the filtrate was concentrated in vacuum to give as colorless oil (1.90 g, crude). LCMS m/z=164.2 (M+1).

Step 5. 5-[(1R)-1-(3,5-dichloro-4-pyridyl)ethoxy]-3-[5-fluoro-6-[(2S,3R)-2-methyl-3-(methylsulfonylmethyl)azetidin-1-yl]-3-pyridyl]-1-tetrahydropyran-2-yl-indazole. To a solution of 5-[(1R)-1-(3,5-dichloro-4-pyridyl)ethoxy]-3-(5,6-difluoro-3-pyridyl)-1-tetrahydropyran-2-yl-indazole (1.00 g, 1.98 mmol, 1.0 eq) and (2S,3R)-2-methyl-3-(methylsulfonylmethyl)azetidine TFA (823 mg, crude) in DMSO (10 mL) was added DIEA (1.79 g, 13.85 mmol, 7.0 eq) at room temperature. The mixture was stirred for 5 h at 100° C.

After the reaction was completed, the mixture was poured into water (30 mL), and extracted with EtOAc (30 mL×2). The combined organic layers were washed with brine (50 mL), dried over Na₂SO₄, and concentrated. The crude product was purified by silica gel column (Petroleum ether/Ethyl acetate=2/1) to give a white solid (1.1 mg, 86% yield). LCMS m/z=648.3 (M+1).

Step 6. 5-((R)-1-(3,5-dichloropyridin-4-yl)ethoxy)-3-(5-fluoro-6-((2S,3R)-2-methyl-3-((methylsulfonyl)methyl)azetidin-1-yl)pyridin-3-yl)-1H-indazole. To a solution of 5-[(1R)-1-(3,5-dichloro-4-pyridyl)ethoxy]-3-[5-fluoro-6-[(2S,3R)-2-methyl-3-(methylsulfonylmethyl)azetidin-1-yl]-3-pyridyl]-1-tetrahydropyran-2-yl-indazole (1.00 g, 1.54 mmol, 1.0 eq) in DCM (20 mL) was added TFA (6 mL) at 0° C. The resulting mixture was stirred at 20° C. for 5 h. After the reaction was completed, the mixture was treated with NaHCO₃ solution to adjust pH to 7-8, and was extracted with DCM (100 mL). The combined organic layers were washed with brine (100 mL), dried over Na₂SO₄ and concentrated in vacuum. The crude was purified by silica gel column (Dichloromethane/Methanol=15/1) and further purified by Prep-HPLC (Prep-C18, 5 μM Triart column, 20×150 mm, YMC-Actus; gradient elution of 45% MeCN in water to 60% MeCN in water over a 8 min period, where both solvents contain 0.05% NH₃·H₂O) to give an off-white solid (644 mg, 74% yield). LCMS m/z=564.1 (M+H); 1HNMR (400 MHz, DMSO-d6) δ 13.12 (s, 1H), 8.58 (s, 2H), 8.37 (t, J=1.6 Hz, 1H), 7.74 (dd, J=13.2 Hz, 2.0 Hz, 1H), 7.49 (d, J=9.2 Hz, 1H), 7.19 (d, J=1.6 Hz, 1H), 7.11 (dd, J=8.8 Hz, 2.4 Hz, 1H), 6.15 (q, J=6.8 Hz, 1H), 4.39-4.28 (m, 2H), 3.91-3.86 (m, 1H), 3.61-3.50 (m, 2H), 3.00 (s, 3H), 2.89-2.83 (m, 1H), 1.77 (d, J=6.8 Hz, 3H), 1.54 (d, J=6.0 Hz, 3H).

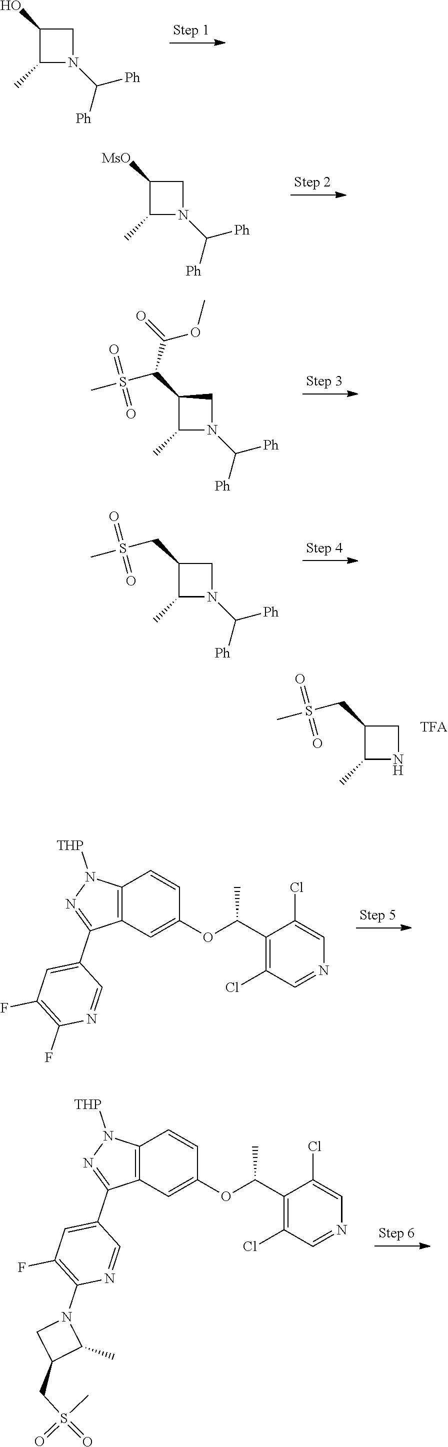

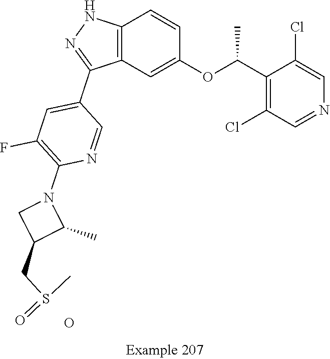

### Example 207. 5-((R)-1-(3,5-dichloropyridin-4-yl)ethoxy)-3-(5-fluoro-6-((2R,3S)-2-methyl-3-((methylsulfonyl)methyl)azetidin-1-yl)pyridin-3-yl)-1H-indazole

Step 1. [(2R,3S)-1-benzhydryl-2-methyl-azetidin-3-yl]methanesulfonate. To a solution (2R,3S)-1-benzhydryl-2-methyl-azetidin-3-ol (15.5 g, 61.18 mmol, 1.0 eq) and TEA (18.9 g, 183.56 mmol, 3.0 eq) in DCM (100 mL) was added MsCl (10.50 g, 91.77 mmol, 1.5 eq) in DCM (30 mL) dropwise at 0° C. The solution was stirred for 3 h at rt, then was diluted with water (100 mL) and extracted with DCM (200 mL×3). The combined organic layers were washed with brine (200 mL×2), dried over Na₂SO₄ and concentrated in vacuum. The crude product was purified by silica gel flash column chromatography (Petroleum ether/EtOAc=5/1) to give a white solid (17 g, 84% yield). LCMS m/z=332.2 (M+1).

Step 2. Methyl 2S-2-[(2R,3S)-1-benzhydryl-2-methyl-azetidin-3-yl]-2-methylsulfonyl-acetate. To a solution of methyl 2-methylsulfonylacetate (5.0 g, 32.86 mmol, 1.0 eq) in DMF (50 mL) was added NaH (1.0 g, 42.72 mmol, 1.3 eq) at 0° C. After stirring for 30 min, [(2R,3S)-1-benzhydryl-2-methyl-azetidin-3-yl]methanesulfonate (14.2 g, 42.72 mmol, 1.3 eq) was added and the resulting mixture stirred at 80° C. for 3 h. Saturated NH₄Cl solution (60 mL) was added to the mixture and extracted with EtOAc (100 mL×3). The combined organic layers were dried over Na₂SO₄ and concentrated in vacuo. The crude product was purified by silica gel flash column chromatography (Petroleum ether/EtOAc=2/1) to give a yellow oil (5.4 g, 42% yield). LCMS m/z=388.2 (M+1).

Step 3. (2R,3S)-1-benzhydryl-2-methyl-3-(methylsulfonylmethyl)azetidine. To a solution of methyl 2S-2-[(2R,3S)-1-benzhydryl-2-methyl-azetidin-3-yl]-2-methylsulfonyl-acetate (5.4 g, 13.94 mmol, 1.0 eq) in DMA (280 mL) was added LiCl (2.95 g, 69.68 mmol, 5.0 eq) at 0° C. The mixture was stirred at 150° C. for 3 h, was cooled down to rt, water (500 mL) was added, and extracted with EtOAc (300 mL×3). The combined organic layers were dried over Na₂SO₄ and concentrated in vacuo. The crude product was purified by silica gel flash column chromatography (Petroleum ether/EtOAc=2/1) to give a yellow solid (2.6 g, 57% yield). LCMS m/z=330.2 (M+1).

Step 4. (2R,3S)-2-methyl-3-(methylsulfonylmethyl)azetidine TFA salt. To a solution of 2R,3S)-1-benzhydryl-2-methyl-3-(methylsulfonylmethyl)azetidine (2.6 g, 7.89 mmol, 1.0 eq) in MeOH (30 mL) was added TFA (2.7 g, 23.68 mmol, 3.0 eq) and Pd(OH)₂ (1.1 g) in portions under N₂ protection. The suspension was degassed under vacuum and purged with H₂ three times. The mixture was stirred at rt for 16 h under H₂ balloon, After the reaction was completed, the solid was filtered out and the filtrate was concentrated to give a colorless oil (2.6 g, crude). LCMS m/z=164.1 (M+1).

Step 5. 5-[(1R)-1-(3,5-dichloro-4-pyridyl)ethoxy]-3-[5-fluoro-6-[(2R,3S)-2-methyl-3-(methylsulfonylmethyl)azetidin-1-yl]-3-pyridyl]-1-tetrahydropyran-2-yl-indazole. To a solution of 2R,3S)-2-methyl-3-(methylsulfonylmethyl)azetidine TFA salt (630 mg, 3.86 mmol, 1.5 eq) and 5-[(1R)-1-(3,5-dichloro-4-pyridyl)ethoxy]-3-(5,6-difluoro-3-pyridyl)-1-tetrahydropyran-2-yl-indazole (1.3 g, 2.57 mmol, 1.0 eq) in DMSO (20 mL) was added DIEA (5.0 g, 38.55 mmol, 15.0 eq). The reaction mixture was stirred at 105° C. for 5 h. After the reaction was completed, the mixture was diluted with EtOAc (150 mL) and washed with brine (50 mL×5). The organic layer was dried over Na₂SO₄ and concentrated. The crude product was purified by silica gel flash column chromatography (Petroleum ether/EtOAc=1/1) to give a white solid (1.1 g, 66% yield). LCMS m/z=648.2 (M+1).

Step 6. 5-((R)-1-(3,5-dichloropyridin-4-yl)ethoxy)-3-(5-fluoro-6-((2R,3S)-2-methyl-3-((methylsulfonyl)methyl)azetidin-1-yl)pyridin-3-yl)-1H-indazole. To a solution of 5-[(1R)-1-(3,5-dichloro-4-pyridyl)ethoxy]-3-[5-fluoro-6-[(2R,3S)-2-methyl-3-(methylsulfonylmethyl)azetidin-1-yl]-3-pyridyl]-1-tetrahydropyran-2-yl-indazole (1.1 g, 1.7 mmol, 1.0 eq) in DCM (20 mL) and MeOH (4 mL) was added HCl/EtOAc (4M, 5 mL). The solution was stirred at rt for 5 h and was concentrated in vacuum. The crude product was basified with the saturate NaHCO₃ solution, which was extracted with EtOAc (60 mL×3). The combined organic layers were washed with brine (100 mL×2), dried over Na₂SO₄ and concentrated in vacuo. The residue was purified by Prep-HPLC (Prep-C18, 5 μM Triart column, 20×150 mm, YMC-Actus; gradient elution of 40% MeCN in water to 50% MeCN in water over a 15 min period, where both solvents contain 0.05% NH₃·H₂O) to give a white solid (510 mg, 53% yield). LCMS m/z=564.2 (M+H); 1HNMR (400 MHz, DMSO-d6) δ 13.12 (brs, 1H), 8.57 (s, 2H), 8.39 (t, J=1.6 Hz, 1H), 7.72 (dd, J=13.2 Hz, 1.6 Hz, 1H), 7.48 (d, J=9.2 Hz, 1H), 7.20 (d, J=2.0 Hz, 1H), 7.10 (dd, J=9.2 Hz, 2.8 Hz, 1H), 6.14 (q, J=6.4 Hz, 1H), 4.38-4.29 (m, 2H), 3.89-3.86 (m, 1H), 3.61-3.51 (m, 2H), 3.00 (s, 3H), 2.88-2.83 (m, 1H), 1.76 (d, J=6.4 Hz, 3H), 1.53 (d, J=6.4 Hz, 3H).

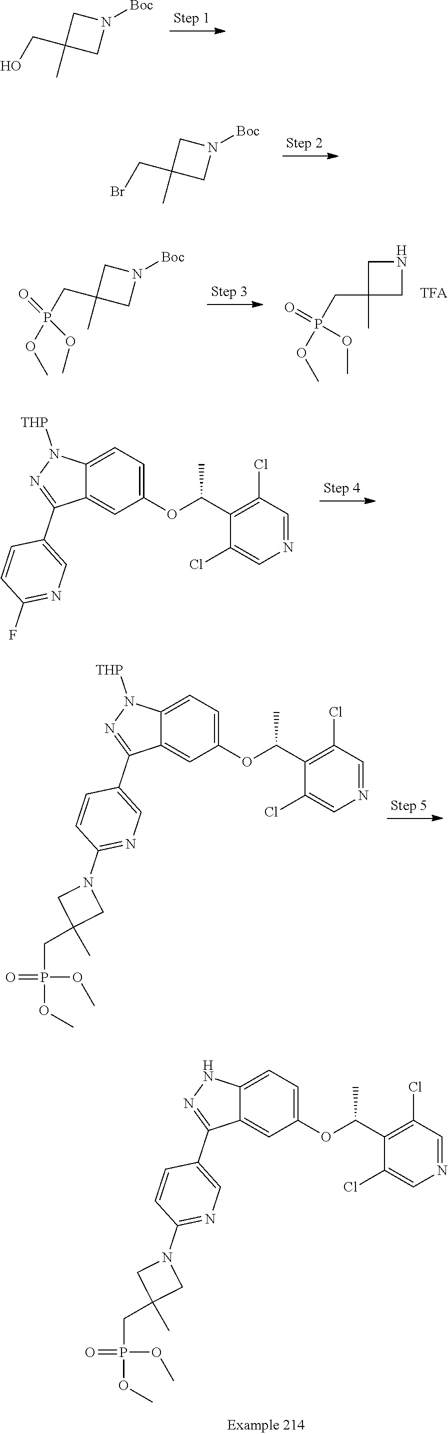

### Example 214. (R)-dimethyl ((1-(5-(5-(1-(3,5-dichloropyridin-4-yl)ethoxy)-1H-indazol-3-yl)pyridin-2-yl)-3-methylazetidin-3-yl)methyl)phosphonate

Step 1. tert-butyl 3-(bromomethyl)-3-methyl-azetidine-1-carboxylate. To a solution of tert-butyl 3-(hydroxymethyl)-3-methyl-azetidine-1-carboxylate (4.0 g, 19.87 mmol, 1.0 eq) and CBr₄ (13.2 g, 39.74 mmol, 2.0 eq) in DCM (100 mL) was added PPh₃ (10.4 g, 39.74 mmol, 2.0 eq) under nitrogen atmosphere at 0° C. The reaction mixture was stirred at rt for 5 h and then was poured into ice-water and extracted with DCM (100 mL×2). The combined organic layers were washed with brine (100 mL), dried over Na₂SO₄, and concentrated in vacuo. The crude product was purified by silica gel flash column chromatography (Petroleum Ether/EtOAc=5/1) to give a yellow liquid (2.8 g, 53% yield). LCMS m/z=264.3 (M+1).

Step 2. tert-butyl 3-(dimethoxyphosphorylmethyl)-3-methyl-azetidine-1-carboxylate. To a solution of methoxyphosphonoyloxymethane (208 mg, 1.89 mmol, 1.0 eq) in dry DMF (5 mL) was added t-BuOK (212 mg, 1.89 mmol, 1.0 eq) under nitrogen atmosphere at 0° C. The reaction mixture was stirred at 0° C. for 0.5 h, then the tert-butyl 3-(bromomethyl)-3-methyl-azetidine-1-carboxylate (500 mg, 1.89 mmol, 1.0 eq) was added and the mixture was stirred at 70° C. for another 16 h. The reaction was diluted with EtOAc (50 mL) and washed with brine (10 mL), dried over Na₂SO₄ and concentrated in vacuo. The crude product was purified by silica gel flash column chromatography (Petroleum Ether) to give a yellow liquid (400 mg, crude). LCMS m/z=294.3 (M+1).

Step 3. 3-(dimethoxyphosphorylmethyl)-3-methyl-azetidine TFA. To a solution of tert-butyl 3-(dimethoxyphosphorylmethyl)-3-methyl-azetidine-1-carboxylate (200 mg, 0.68 mmol, 1.0 eq) in DCM (2 mL) was added TFA (1 ml) at rt. The reaction was stirred at rt for 3 h and then concentrated to give a yellow oil (200 mg, crude). LCMS m/z=194.3 (m+1).

Step 4. 5-[(1R)-1-(3,5-dichloro-4-pyridyl)ethoxy]-3-[6-[3-(dimethoxyphosphorylmethyl)-3-methyl-azetidin-1-yl]-3-pyridyl]-1-tetrahydropyran-2-yl-indazole. To a solution of 5-[(1R)-1-(3,5-dichloro-4-pyridyl)ethoxy]-3-(6-fluoro-3-pyridyl)-1-tetrahydropyran-2-yl-indazole (200 mg, 0.41 mmol, 1.0 eq) and 3-(dimethoxyphosphorylmethyl)-3-methyl-azetidine TFA (158 mg, 0.82 mmol, 2.0 eq) in DMSO (3 mL) was added DIEA (529 mg, 4.1 mmol, 5.0 eq), the solution was stirred at 100° C. for 16 h. The reaction was diluted with EtOAc (30 mL) and washed with brine (10 mL×2) and was dried over Na₂SO₄ and concentrated. The crude product was purified by Prep-TLC (DCM/MeOH=10/1) to give a yellow solid (80 mg, 29.5% yield). LCMS m/z=660.3 (M+1).

Step 5. (R)-dimethyl ((1-(5-(5-(1-(3,5-dichloropyridin-4-yl)ethoxy)-1H-indazol-3-yl)pyridin-2-yl)-3-methylazetidin-3-yl)methyl)phosphonate. To a solution of 5-[(1R)-1-(3,5-dichloro-4-pyridyl)ethoxy]-3-[6-[3-(dimethoxyphosphorylmethyl)-3-methyl-azetidin-1-yl]-3-pyridyl]-1-tetrahydropyran-2-yl-indazole (80 mg, 0.12 mmol, 1.0 eq) in DCM (3 mL) was added TFA (2 mL) and the reaction was stirred at rt for 3 h. After the reaction completion, the solvent was removed, the residue was diluted with DCM (20 ml) and washed with the saturated NaHCO₃ solution. The organic layer was dried over Na₂SO₄ and concentrated. The crude product was purified by Prep-HPLC (Prep-C18, 5 μM Triart column, 20×150 mm, YMC-Actus; gradient elution of 40% MeCN in water to 70% MeCN in water over a 10 min period, where both solvents contain 0.05% NH₃·H₂O) to give an off-white solid (31 mg, 45% yield). LCMS m/z=576.2 (M+H); 1HNMR (400 MHz, DMSO-d6) δ 13.00 (s, 1H), 8.60 (s, 2H), 8.50 (d, J=2.0 Hz, 1H), 7.85 (d, J=6.4 Hz, 1H), 7.46 (d, J=9.2 Hz, 1H), 7.16 (s, 1H), 7.09 (dd, J=9.2 Hz, 2.4 Hz, 1H), 6.54 (d, J=8.4 Hz, 1H), 6.11 (q, J=6.8 Hz, 1H), 3.94 (d, J=8.0 Hz, 2H), 3.73 (d, J=7.6 Hz, 2H), 3.67 (s, 3H), 3.64 (s, 3H), 2.31-2.26 (d, J=18.0 Hz, 2H), 1.76 (d, J=6.4 Hz, 3H), 1.49 (s, 3H).

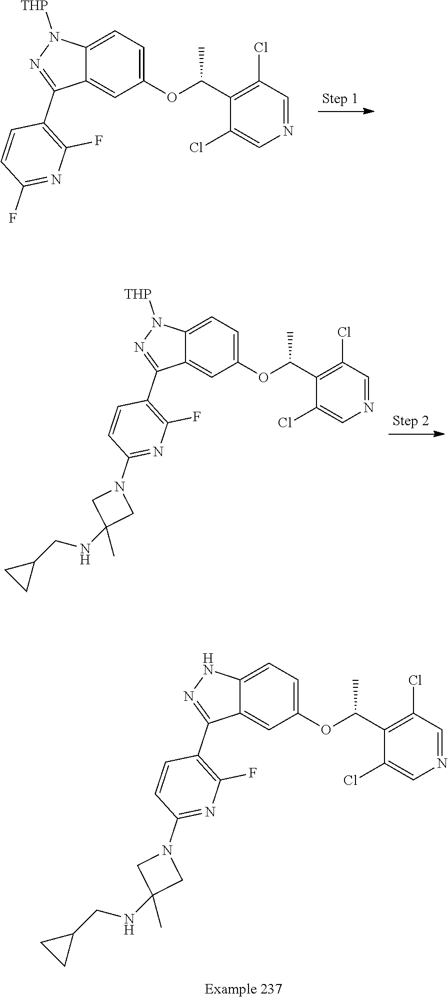

### Example 237. (R)—N-(cyclopropylmethyl)-1-(5-(5-(1-(3,5-dichloropyridin-4-yl)ethoxy)-1H-indazol-3-yl)-6-fluoropyridin-2-yl)-3-methylazetidin-3-amine

Step 1. N-(cyclopropylmethyl)-1-[5-[5-[(1R)-1-(3,5-dichloro-4-pyridyl)ethoxy]-1-tetrahydropyran-2-yl-indazol-3-yl]-6-fluoro-2-pyridyl]-3-methyl-azetidin-3-amine. To a solution of 5-[(1R)-1-(3,5-dichloro-4-pyridyl)ethoxy]-3-(2,6-difluoro-3-pyridyl)-1-tetrahydropyran-2-yl-indazole (400 mg, 0.79 mmol, 1.0 eq) and N-(cyclopropylmethyl)-3-methyl-azetidin-3-amine HCl salt (222 mg, 1.58 mmol, 2.0 eq) in DMSO (5 mL) was added DIEA (409 mg, 3.17 mmol, 4.0 eq) at room temperature. The mixture was stirred for 3 h at 90° C., poured into water (30 mL), and was extracted with EtOAc (30 mL×2). The combined organic layers were washed with brine (60 mL), dried over Na₂SO₄ and concentrated. The crude product was purified by silica gel column (Petroleum ether/Ethyl acetate=1/1) to give a white solid (170 mg, 34% yield). LCMS m/z=625.3 (M+1).

Step 2. (R)—N-(cyclopropylmethyl)-1-(5-(5-(1-(3,5-dichloropyridin-4-yl)ethoxy)-1H-indazol-3-yl)-6-fluoropyridin-2-yl)-3-methylazetidin-3-amine. To a solution of N-(cyclopropylmethyl)-1-[5-[5-[(1R)-1-(3,5-dichloro-4-pyridyl)ethoxy]-1-tetrahydropyran-2-yl-indazol-3-yl]-6-fluoro-2-pyridyl]-3-methyl-azetidin-3-amine (150 mg, 0.24 mmol, 1.0 eq) in DCM (6 mL) was added TFA (2 mL) at 0° C. The resulting mixture was stirred at rt for 4 h, then treated with NaHCO₃ aqueous solution to adjust pH to 7-8 and extracted with DCM (20 mL×3). The combined organic layers were washed with brine (50 mL), dried over Na₂SO₄, and concentrated in vacuum. The crude product was purified by Prep-TLC (dichloromethane/methanol=10/1) to give a white solid (63 mg, 49% yield). LCMS m/z=541.3 (M+H); 1HNMR (400 MHz, DMSO-d6) δ 13.10 (s, 1H), 8.58 (s, 2H), 7.83 (t, J=8.4 Hz, 1H), 7.47 (d, J=9.2 Hz, 1H), 7.11 (dd, J=8.8 Hz, 2.4 Hz, 1H), 6.91 (s, 1H), 6.39 (dd, J=8.0 Hz, 1.6 Hz, 1H), 6.03 (q, J=6.4 Hz, 1H), 3.87 (d, J=8.0 Hz, 2H), 3.76 (dd, J=8.0 Hz, 2.0 Hz, 2H), 2.42 (d, J=6.4 Hz, 2H), 1.74 (d, J=6.4 Hz, 3H), 1.41 (s, 3H), 0.90-0.83 (m, 1H), 0.44-0.39 (m, 2H), 0.16-0.12 (m, 2H).

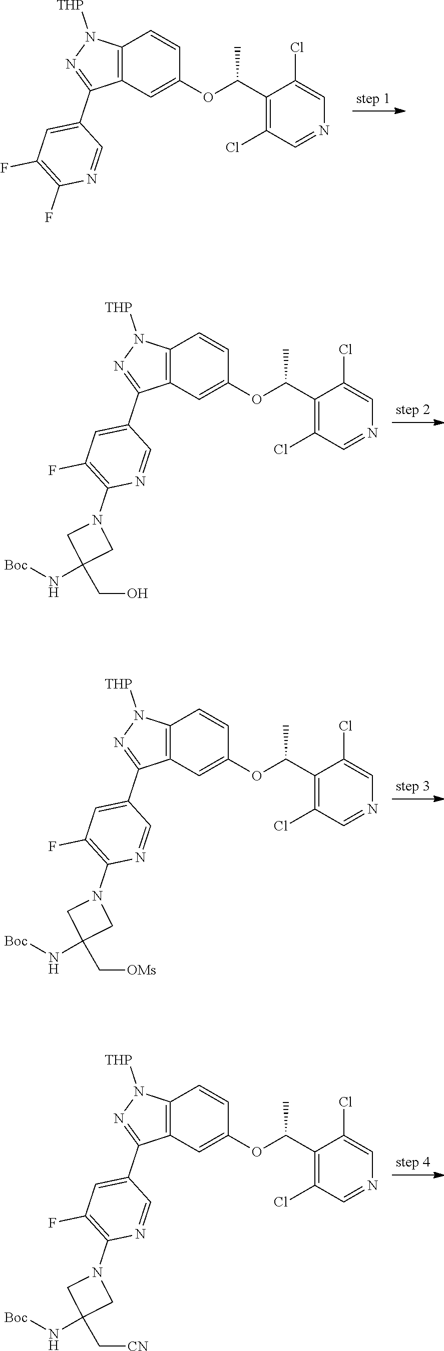

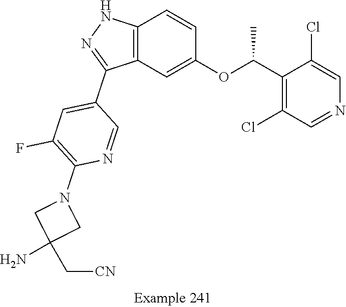 [SMILES: C[C@@H](Oc1ccc2[nH]nc(-c3cnc(N4CC(N)(CC#N)C4)c(F)c3)c2c1)c1c(Cl)cncc1Cl]

### Example 241. (R)-2-(3-amino-1-(5-(5-(1-(3,5-dichloropyridin-4-yl)ethoxy)-1H-indazol-3-yl)-3-fluoropyridin-2-yl)azetidin-3-yl)acetonitrile

Step 1. tert-butyl N-[1-[5-[5-[(1R)-1-(3,5-dichloro-4-pyridyl)ethoxy]-1-tetrahydropyran-2-yl-indazol-3-yl]-3-fluoro-2-pyridyl]-3-(hydroxymethyl)azetidin-3-yl]carbamate. To a solution of 5-[(1R)-1-(3,5-dichloro-4-pyridyl)ethoxy]-3-(5,6-difluoro-3-pyridyl)-1-tetrahydropyran-2-yl-indazole (200 mg, 0.396 mmol, 1.0 eq) and tert-butyl N-[3-(hydroxymethyl)azetidin-3-yl]carbamate (160 mg, 0.792 mmol, 2.0 eq) in DMSO (5 mL) was added DIEA (255 mg, 1.98 mmol, 5.0 eq). The reaction mixture was stirred for 16 h at 100° C. and then mixture was diluted with EtOAc (20 mL), washed with brine (10 mL×3) and concentrated. The product was purified by silica gel flash column chromatography (DCM/MeOH=15/1) to give a yellow solid (265 mg, 97% yield). LCMS m/z=687.4 (M+1).

Step 2. [3-(tert-butoxycarbonylamino)-1-[5-[5-[(1R)-1-(3,5-dichloro-4-pyridyl)ethoxy]-1-tetrahydropyran-2-yl-indazol-3-yl]-3-fluoro-2-pyridyl]azetidin-3-yl]methyl methanesulfonate. To a solution of tert-butyl N-[1-[5-[5-[(1R)-1-(3,5-dichloro-4-pyridyl)ethoxy]-1-tetrahydropyran-2-yl-indazol-3-yl]-3-fluoro-2-pyridyl]-3-(hydroxymethyl)azetidin-3-yl]carbamate (260 mg, 0.378 mmol, 1.0 eq) and TEA (115 mg, 1.13 mmol, 3.0 eq) in DCM (10 mL) was added MsCl (60 mg, 0.529 mmol, 1.4 eq). The reaction mixture was stirred for 16 h at r. under N₂. After the reaction was completed, H₂O (20 mL) was added, and the aqueous layer extracted with DCM (30 mL×2). The combined organic layers were concentrated. And the crude product was purified by silica gel flash column chromatography (DCM/MeOH=15/1) to give an off-white solid (240 mg, 83% yield). LCMS m/z=765.3 (M+1).

Step 3. tert-butyl N-[3-(cyanomethyl)-1-[5-[5-[(1R)-1-(3,5-dichloro-4-pyridyl)ethoxy]-1-tetrahydropyran-2-yl-indazol-3-yl]-3-fluoro-2-pyridyl]azetidin-3-yl]carbamate. To a solution of tert-butyl N-[3-(cyanomethyl)-1-[5-[5-[(1R)-1-(3,5-dichloro-4-pyridyl)ethoxy]-1-tetrahydropyran-2-yl-indazol-3-yl]-3-fluoro-2-pyridyl]azetidin-3-yl]carbamate (180 mg, 0.235 mmol, 1.0 eq) and TBAF/THF (0.5 mL, 1 mol/L) in ACN (5 mL) was added K₂CO₃ (97 mg, 0.705 mmol, 3.0 eq) and TMSCN (35 mg, 0.353 mmol, 1.5 eq). The reaction mixture was stirred for 2 h at 80° C. under N₂. After the reaction was completed, the mixture was diluted with EtOAc (20 mL) and washed with H2O (10 mL×3). The organic layer was concentrated and the crude product was purified by silica gel flash column chromatography (DCM/MeOH=15/1) to give a yellow solid (93 mg, 56% yield). LCMS m/z=696.3 (M+1).

Step 4. (R)-2-(3-amino-1-(5-(5-(1-(3,5-dichloropyridin-4-yl)ethoxy)-1H-indazol-3-yl)-3-fluoropyridin-2-yl)azetidin-3-yl)acetonitrile. To a solution of tert-butyl N-[3-(cyanomethyl)-1-[5-[5-[(1R)-1-(3,5-dichloro-4-pyridyl)ethoxy]-1-tetrahydropyran-2-yl-indazol-3-yl]-3-fluoro-2-pyridyl]azetidin-3-yl]carbamate (83 mg, 0.119 mmol, 1.0 eq) in DCM (2 mL) was added TFA (0.5 mL), the solution was stirred at rt for 5 h. The reaction mixture was concentrated in vacuum and the crude product was treated with DCM (10 mL), adjusted pH to 7-8 by addition of the saturate NaHCO₃ solution and was extracted with DCM (10 mL×3). The combined organic layers were washed with brine (10 mL), dried over Na₂SO₄ and concentrated. The crude product was purified by Pre-HPLC (Prep-C18, 5 μM Triart column, 20×150 mm, YMC-Actus; gradient elution of 25% MeCN in water to 60% MeCN in water over a 12 min period, where both solvents contain 0.05% NH₃·H₂O) to give an off-white solid (22 mg, 37% yield). LCMS m/z=512.2 (M+H); 1HNMR (400 MHz, DMSO-d6) δ 13.10 (s, 1H), 8.54 (s, 2H), 8.35 (s, 1H), 7.69 (d, J=13.2 Hz, 1H), 7.44 (d, J=8.8 Hz, 1H), 7.17 (s, 1H), 7.06 (d, J=9.2 Hz, 2.0 Hz, 1H), 6.11 (q, J=6.4 Hz, 1H), 4.02 (d, J=7.6 Hz, 2H), 3.92 (d, J=8.0 Hz, 2H), 2.99 (s, 2H), 1.72 (d, J=6.8 Hz, 3H).

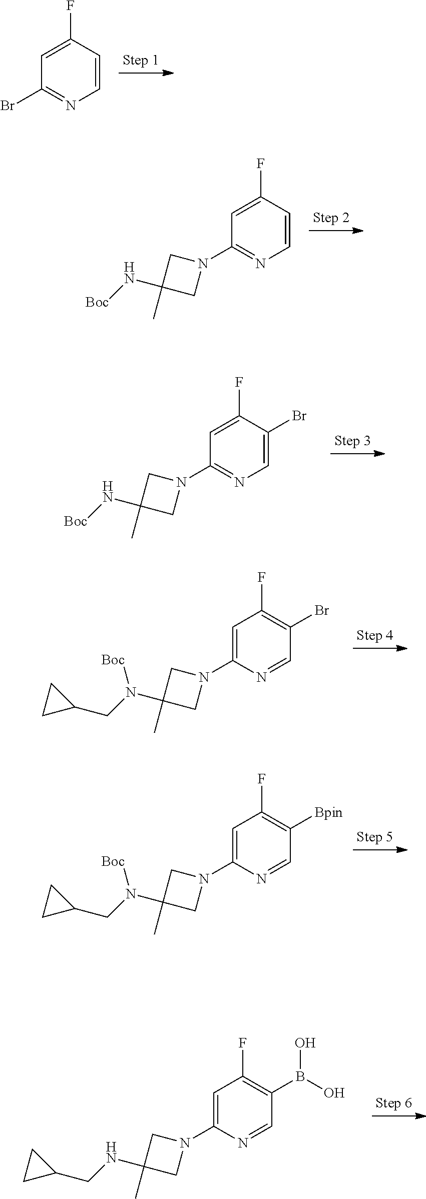

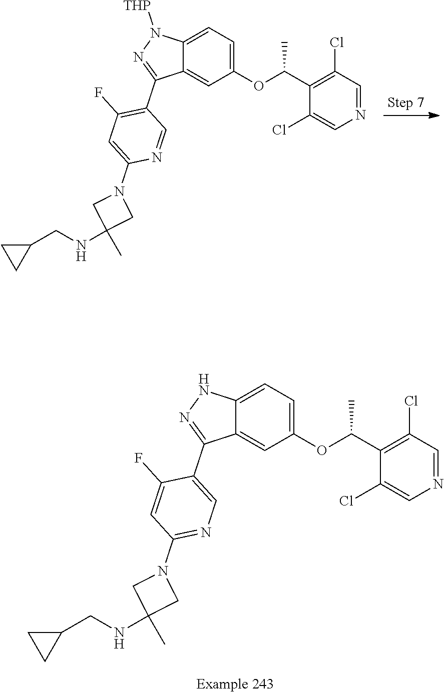

### Example 243. (R)—N-(cyclopropylmethyl)-1-(5-(5-(1-(3,5-dichloropyridin-4-yl)ethoxy)-1H-indazol-3-yl)-4-fluoropyridin-2-yl)-3-methylazetidin-3-amine

Step 1. tert-butyl N-[1-(4-fluoro-2-pyridyl)-3-methyl-azetidin-3-yl]carbamate. To a solution of 2-bromo-4-fluoro-pyridine (3.6 g, 20.46 mmol, 1.0 eq) and tert-butyl N-(3-methylazetidin-3-yl)carbamate (4.96 g, 26.6 mmol, 1.3 eq) in toluene (30 mL) was added BINAP (2.55 g, 8.18 mmol, 0.4 eq), Cs₂CO₃ (19.92 g, 61.36 mmol, 3.0 eq) and Pd2(dba)₃ (3.74 g, 4.1 mmol, 0.2 eq). The reaction mixture was stirred at 80° C. for 6 h under N₂ protection. The reaction mixture was cooled to rt and concentrated in vacuum. The crude product was purified by silica gel chromatography (Petroleum Ether/EtOAc=2/1) to give a yellow oil (1.8 g, 31% yield). LCMS m/z=282.2 (M+1).

Step 2. tert-butyl N-[1-(5-bromo-4-fluoro-2-pyridyl)-3-methyl-azetidin-3-yl]carbamate. To a solution of tert-butyl N-[1-(4-fluoro-2-pyridyl)-3-methyl-azetidin-3-yl]carbamate (1.0 g, 3.55 mmol, 1.0 eq) in ACN (15 mL) was added NBS (633 mg, 3.55 mmol, 1.0 eq) batch wise at 0° C. The reaction mixture was stirred at 0° C. for 1 h. The mixture was concentrated in vacuum and the product purified by silica gel column (Petroleum Ether/EtOAc=5/1) to give a yellow oil (900 mg, 70% yield). LCMS m/z=360.1 (M+1); 1H NMR (400 MHz, DMSO-d6) δ 8.21 (d, J=10.0 Hz, 1H), 7.42 (brs, 1H), 6.48 (d, J=10.8 Hz, 1H), 3.99-3.97 (m, 2H), 3.76-3.74 (m, 2H), 1.47 (s, 3H), 1.38 (s, 9H).

Step 3. tert-butyl N-[1-(5-bromo-4-fluoro-2-pyridyl)-3-methyl-azetidin-3-yl]-N-(cyclopropylmethyl)carbamate. To a solution of tert-butyl N-[1-(5-bromo-4-fluoro-2-pyridyl)-3-methyl-azetidin-3-yl]carbamate (900 mg, 2.5 mmol, 1.0 eq) in DMF (15 mL) was added NaH (120 mg, 5.0 mmol, 2.0 eq) batch wise at 0° C. After stirring for 0.5 h at 0° C., bromomethylcyclopropane (506 mg, 3.75 mmol, 1.5 eq) in DMF (1 mL) was added and stirred at rt for 1 h. The reaction mixture was diluted with EtOAc (200 mL) and washed with water (100 mL×2). The organic layer was concentrated in vacuum and the crude product was purified by silica gel chromatography (Petroleum Ether/EtOAc=5/1) to give a yellow oil (800 mg, 72% yield). LCMS m/z=414.2 (M+1).

Step 4. tert-butyl N-(cyclopropylmethyl)-N-[1-[4-fluoro-5-(4,4,5,5-tetramethyl-1,3,2-dioxaborolan-2-yl)-2-pyridyl]-3-methyl-azetidin-3-yl]carbamate. To a solution of tert-butyl N-[1-(5-bromo-4-fluoro-2-pyridyl)-3-methyl-azetidin-3-yl]-N-(cyclopropylmethyl)carbamate (450 mg, 1.09 mmol, 1.0 eq) and BPD (2.76 mg, 10.86 mmol, 10.0 eq) in dioxane (7 mL) was added KOAc (319 mg, 3.26 mmol, 3.0 eq) and Pd(dppf)Cl₂ (88 mg, 0.11 mmol, 0.1 eq). The reaction mixture was stirred at 90° C. for 6 h under N₂ protection. After cooled to room temperature, the solvent was removed and the crude product was purified by prep-MPLC (Prep-C18, 20-45DM, 120 g, Tianjin Bonna-Agela Technologies; gradient elution of 40% MeCN in water to 80% MeCN in water, over a 10 min period) to give a yellow oil (300 mg, 60% yield). LCMS m/z=462.3 (M+1).

Step 5. [6-[3-(cyclopropylmethylamino)-3-methyl-azetidin-1-yl]-4-fluoro-3-pyridyl]boronic acid. To a solution of tert-butyl N-(cyclopropylmethyl)-N-[1-[4-fluoro-5-(4,4,5,5-tetramethyl-1,3,2-dioxaborolan-2-yl)-2-pyridyl]-3-methyl-azetidin-3-yl]carbamate (300 mg, 0.65 mmol, 1.0 eq) in DCM (2 mL) was added TFA (1 mL). The reaction mixture was stirred at rt for 2 h. After the reaction was completed, the reaction mixture was concentrated. The crude product was purified by prep-MPLC (Prep-C18, 20-45 uM, 120 g, Tianjin Bonna-Agela Technologies; gradient elution of 25% MeCN in water to 40% MeCN in water, over a 10 min period) to give a yellow oil (162 mg, 88.1% yield). LCMS m/z=280.3 (M+1).

Step 6. N-(cyclopropylmethyl)-1-[5-[5-[(1R)-1-(3,5-dichloro-4-pyridyl)ethoxy]-1-tetrahydropyran-2-yl-indazol-3-yl]-4-fluoro-2-pyridyl]-3-methyl-azetidin-3-amine. To a solution of 5-[(1R)-1-(3,5-dichloro-4-pyridyl)ethoxy]-3-iodo-1-tetrahydropyran-2-yl-indazole (200 mg, 0.39 mmol, 1.0 eq) and [6-[3-(cyclopropylmethylamino)-3-methyl-azetidin-1-yl]-4-fluoro-3-pyridyl]boronic acid (162 mg, 0.58 mmol, 1.5 eq) in dioxane (4 mL) and water (0.2 mL) was added K₂CO₃ (160 mg, 1.16 mmol, 3.0 eq) and Pd(dppf)Cl₂ (32 mg, 0.039 mmol, 0.1 eq). The reaction mixture was stirred at 90° C. for 4 h under N₂ protection. After cooled to room temperature, the solvent was removed and the crude product was purified by prep-TLC (DCM/MeOH=30/1) to give a yellow solid (110 mg, 46% yield). LCMS m/z=625.3 (M+1).

Step 7. (R)—N-(cyclopropylmethyl)-1-(5-(5-(1-(3,5-dichloropyridin-4-yl)ethoxy)-1H-indazol-3-yl)-4-fluoropyridin-2-yl)-3-methylazetidin-3-amine. To a solution of N-(cyclopropylmethyl)-1-[5-[5-[(1R)-1-(3,5-dichloro-4-pyridyl)ethoxy]-1-tetrahydropyran-2-yl-indazol-3-yl]-4-fluoro-2-pyridyl]-3-methyl-azetidin-3-amine (110 mg, 0.16 mmol, 1.0 eq) in DCM (2 mL) was added TFA (1 mL). The reaction mixture was stirred at rt for 2 h. After the reaction was completed, the reaction mixture was concentrated and the crude product was treated with MeOH (3 mL), solid NaHCO₃ (excess) and stirred for 20 minutes at rt. Then DCM (20 mL) was added and the solid was filtered out and filtrate was concentrated. The crude product was purified by Prep-TLC (DCM/MeOH=15/1) to give a white solid (37 mg, 39% yield). LCMS m/z=541.3 (M+H); 1HNMR (400 MHz, DMSO-d6) δ 13.14 (s, 1H), 8.59 (s, 2H), 8.29 (d, J=10.8 Hz, 1H), 7.47 (d, J=8.8 Hz, 1H), 7.10 (dd, J=8.8 Hz, 2.0 Hz, 1H), 6.79 (s, 1H), 6.38 (d, J=12.8 Hz, 1H), 5.98 (q, J=6.4 Hz, 1H), 3.85 (d, J=8.0 Hz, 2H), 3.75 (dd, J=8.0 Hz, 1.2 Hz, 2H), 2.40 (d, J=6.4 Hz, 2H), 1.73 (d, J=6.4 Hz, 3H), 1.41 (s, 3H), 0.89-0.83 (m, 1H), 0.44-0.39 (m, 2H), 0.16-0.12 (m, 2H).

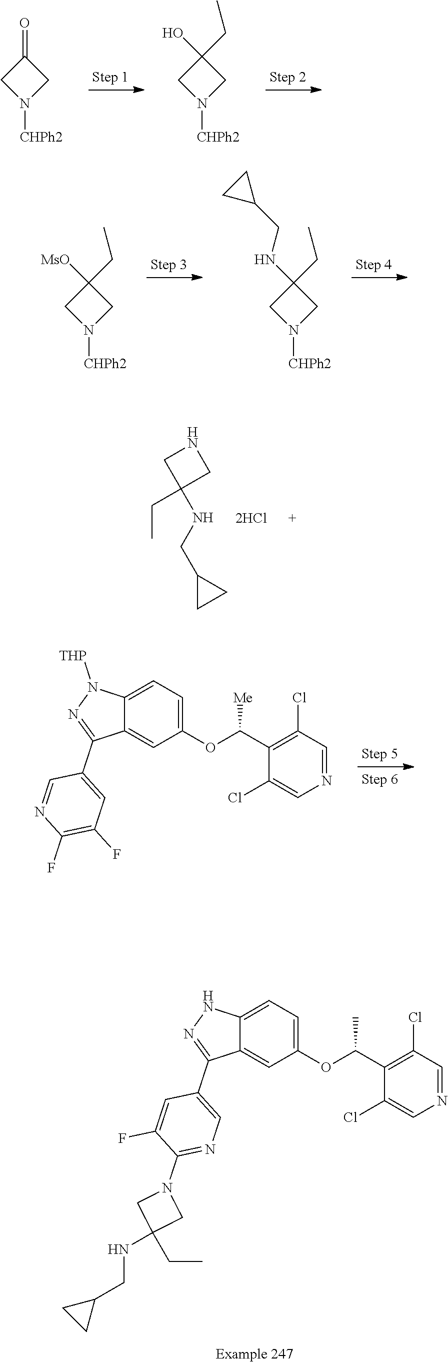

### Example 247

Step 1. 1-Benzhydryl-3-ethylazetidin-3-ol. 3M Ethylmagnesium bromide in diethyl ether (27.0 mL, 81.0 mmol, 1.6 equiv) was added over 30 minutes to a solution of 1-benzhydrylazetidin-3-one (11.86 g, 49.98 mmol, 1 equiv) in tetrahydrofuran (100 mL), while maintaining the temperature below 5° C. during the addition. The reaction was allowed to warm to 15° C. and was stirred for 2 hours. TLC analysis indicated full conversion to the product. The mixture was cooled to 4° C., and sequentially diluted with water (1 mL), saturated ammonium chloride (100 mL) and ethyl acetate (100 mL). The layers were separated, and the aqueous layer was diluted with 30% sodium hydroxide (30 mL), generating a gelatinous precipitate. The mixture was diluted with ethyl acetate (100 mL) and filtered through Celite. The layers were separated, and the organic layer was washed with saturated brine (30 mL), dried over sodium sulfate, filtered and concentrated under reduced pressure. The residue was azeotrope with toluene (50 mL) then dried under vacuum at 40° C. The residue was slurred in hexanes (20 mL) and ethyl acetate (5 mL). Mild heating gave a thin slurry, which was then added slowly to hexanes (80 mL) with stirring. The resulting precipitate was filtered and washed with hexanes (100 mL). The filtrate was filtered and then concentrated under reduced pressure onto silica gel. The residue was purified on a Biotage automated chromatography system (220 g Sorbtech silica gel column), eluting with a gradient of 0 to 100% ethyl acetate in hexanes to give a yellow wax (10.5 g, 79%). LCMS m/z=268 (M+H).

Step 2. 1-Benzhydryl-3-ethylazetidin-3-yl methanesulfonate. 1-Benzhydryl-3-ethylazetidin-3-ol (3.30 g, 12.3 mmol, 1 equiv) in dichloromethane (48 mL) was cooled in an ice-water bath then treated with N,N-diisopropylethylamine (7.0 mL, 30.2 mmol, 3.2 equiv) and methanesulfonic anhydride (2.09 g, 25.8 mmol, 2.0 equiv). The mixture was stirred at room temperature for 2 hours, at which time TLC analysis indicated consumption of starting material. The mixture was sequentially washed with water (20 mL) and saturated brine (20 mL). The organic layer was dried over sodium sulfate, filtered, and concentrated under reduced pressure to give an amber oil (4.5 g, >100%), that was used directly in the next step.

Step 3. 1-Benzhydryl-N-(cyclopropylmethyl)-3-ethylazetidin-3-amine. A mixture of product step 2 (4.59 g, 13.3 mmol, 1 equiv) and cyclopropylmethanamine (9.45 g, 133 mmol, 910 equiv) in 1,4-dioxane (50 mL) was heated at 100° C. in a pressure vessel for 20 hours. After cooling to room temperature, the reaction mixture was diluted with saturated sodium carbonate (30 mL) and ethyl acetate (60 mL). The layers were separated, and the aqueous layer extracted with ethyl acetate (30 mL). The combined organic layers were dried over sodium sulfate and concentrated under reduced pressure. The residue was purified using a Biotage automated chromatography system (Biotage® Sfar KP-Amino D Duo 50 μm 220 g column), eluting with a gradient of 0 to 20% ethyl acetate in hexanes. The fractions containing product were combined and concentrated. The residue was further purified on a Biotage automated chromatography system (Biotage® Sfar Silica 60 μm 50 g column), eluting with a gradient of 0 to 100% ethyl acetate in hexanes to give a colorless oil (1.0 g, 24%). LCMS m/z=321.2 (M+H).

Step 4. N-(Cyclopropylmethyl)-3-ethylazetidin-3-amine dihydrochloride. A mixture of 1-benzhydryl-N-(cyclopropylmethyl)-3-ethylazetidin-3-amine (1 g, 3.12 mmol), 20 wt % palladium hydroxide on carbon (50% wet, 438 mg, 0.31 mmol, 0.1 equiv) and concentrated HCl (1.1 mL, 12.8 mmol, 4.1 equiv) in ethanol (50 mL) was hydrogenated @50 psi at room temperature for 66 hours in a Parr shaker. The reaction mixture was filtered through a pad of Celite, which was washed with 5% water in ethanol (20 mL) and ethanol (20 mL). The filtrate was concentrated under reduced pressure. The residue was triturated with hexanes (2×50 mL), azeotroped with toluene (2×50 mL) and dried under vacuum at 40° C. for 20 hours to give an off-white gummy solid (709 mg, 100%). LCMS m/z=155.2 (M+H) free base.

Step 5. N-(Cyclopropylmethyl)-1-(5-(5-((R)-1-(3,5-dichloropyridin-4-yl)ethoxy)-1-(tetrahydro-2H-pyran-2-yl)-1H-indazol-3-yl)-3-fluoropyridin-2-yl)-3-ethylazetidin-3-amine. A mixture of 5-[(1R)-1-(3,5-dichloro-4-pyridyl)ethoxy]-3-(5,6-difluoro-3-pyridyl)-1-tetrahydropyran-2-yl-indazole (263 mg, 0.52 mmol, 1 equiv), N-(cyclopropylmethyl)-3-ethylazetidin-3-amine dihydrochloride (237 mg, 1.04 mmol, 2 equiv) and N,N-diisopropylethylamine (605 mg, 0.82 mL, 4.68 mmol, 9 equiv) in acetonitrile (20 mL) was heated at 80° C. for 24 hours. The mixture was cooled to room temperature and concentrated. The residue was purified on a Biotage automated chromatography system (Biotage Sfar KP-Amino D Duo 50 μm 28 g column), eluting with a gradient of 0 to 80% ethyl acetate in hexanes to give an off-white solid (196 mg, 59%). LCMS m/z=639.2 (M+H).

Step 6. N-(cyclopropylmethyl)-1-[5-[5-[(1R)-1-(3,5-dichloro-4-pyridyl)ethoxy]-1H-indazol-3-yl]-3-fluoro-2-pyridyl]-3-ethyl-azetidin-3-amine dihydrochloride. N-(Cyclopropylmethyl)-1-(5-(5-((R)-1-(3,5-dichloropyridin-4-yl)ethoxy)-1-(tetrahydro-2H-pyran-2-yl)-1H-indazol-3-yl)-3-fluoropyridin-2-yl)-3-ethylazetidin-3-amine (177 mg, 0.28 mmol, 1.0 equiv) was treated with 2M HCl (1.1 mL, 2.2 mmol, 8 equiv) in ethanol (4.2 mL) at 65° C. After 4 hours, the mixture was cooled to room temperature. The solids were filtered, washed with ethanol (4 mL) and dried at 40° C. under vacuum for 16 hours to give an off-white solid (169 mg, 88%). LCMS m/z=555.2 (M+H) free base; 1H NMR (400 MHz, DMSO-d6) δ=13.20 (br s, 1H), 9.76 (br s, 2H), 8.57 (s, 2H), 8.41 (s, 1H), 7.80 (dd, J=1.7, 13.0 Hz, 1H), 7.49 (d, J=9.0 Hz, 1H), 7.20 (d, J=2.1 Hz, 1H), 7.11 (dd, J=2.1, 9.0 Hz, 1H), 6.14 (q, J=6.7 Hz, 1H), 4.41 (br d, J=9.2 Hz, 2H), 4.15 (br d, J=8.9 Hz, 2H), 2.91-2.83 (m, 2H), 1.99 (q, J=7.3 Hz, 2H), 1.76 (d, J=6.6 Hz, 3H), 1.14-1.04 (m, 4H), 0.64-0.56 (m, 2H), 0.42 (q, J=4.7 Hz, 2H).

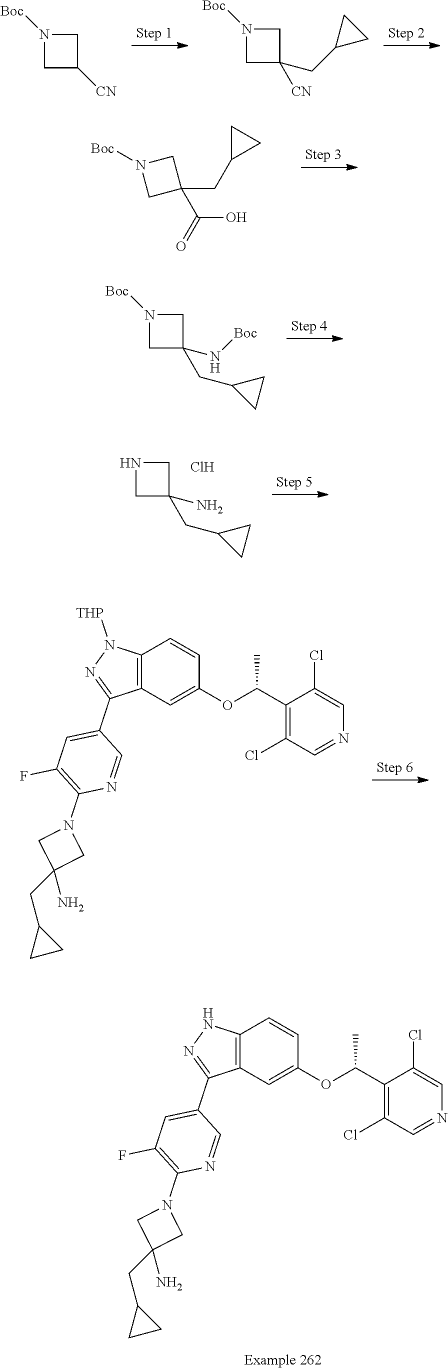

### Example 262. (R)-3-(cyclopropylmethyl)-1-(5-(5-(1-(3,5-dichloropyridin-4-yl)ethoxy)-1H-indazol-3-yl)-3-fluoropyridin-2-yl)azetidin-3-amine

Step 1. tert-butyl 3-cyano-3-(cyclopropylmethyl)azetidine-1-carboxylate. To a solution of tert-butyl 3-cyanoazetidine-1-carboxylate (5.0 g, 27.44 mmol, 1.0 eq) in THE (50 mL) was added LDA (8 mL, 2M, 32.93 mmol, 1.2 eq) dropwise at −78° C. After stirring for 1 h at −78° C., bromomethylcyclopropane (4.82 g, 35.67 mmol, 1.3 eq) in THE (10 mL) was added and stirred at −78° C. for 4 h. The mixture was quenched with the saturated NH₄Cl solution, and extracted with EtOAc (100 mL×2). The combined organic layers were concentrated in vacuo and the crude product was purified by silica gel chromatography (Petroleum Ether/EtOAc=4/1) to give a yellow solid (1.8 g, 28% yield). LCMS m/z=237.4 (M+1).

Step 2. 1-tert-butoxycarbonyl-3-(cyclopropylmethyl)azetidine-3-carboxylic acid. To a solution of tert-butyl 3-cyano-3-(cyclopropylmethyl)azetidine-1-carboxylate (1.8 g, 7.62 mmol, 1.0 eq) in EtOH (18 mL) and H₂O (18 mL) was added KOH (1.71 g, 30.47 mmol, 4.0 eq). The reaction mixture was stirred at 80° C. for 6 h. After the reaction was completed, the pH of the mixture was adjusted to 5 by addition of hydrochloric acid solution (1 M). The precipitated solid was filtered, washed with water, and dried in vacuo to give a yellow solid (1.5 g, 77% yield). LCMS m/z=256.1 (M+1); 1H NMR (400 MHz, CDCl3-d) δ 4.22 (d, J=8.2 Hz, 2H), 3.83 (d, J=8.2 Hz, 2H), 1.86 (d, J=5.6 Hz, 2H), 1.43 (s, 9H), 0.72-0.64 (m, 1H), 0.50-0.43 (m, 2H), 0.14-0.11 (m, 2H).

Step 3. tert-butyl 3-(tert-butoxycarbonylamino)-3-(cyclopropylmethyl)azetidine-1-carboxylate. To a solution of 1-tert-butoxycarbonyl-3-(cyclopropylmethyl)azetidine-3-carboxylic acid (500 mg, 1.96 mmol, 1.0 eq), 4A molecular sieves (500 mg) and TEA (646 mg, 3.92 mmol, 2.0 eq) in dry t-BuOH (20 mL) was added DPPA (646 mg, 2.35 mmol, 1.2 eq) dropwise at 0° C. The reaction mixture was stirred at 80° C. for 4 h. After the reaction was completed, the solid was filtered out and the filtrate was concentrated in vacuum. The crude product was purified by silica gel flash column chromatography (Petroleum Ether/EtOAc=4/1) to give a yellow oil (500 mg, 78% yield). LCMS m/z=327.3 (M+1).

Step 4. 3-(cyclopropylmethyl)azetidin-3-amine HCl. A mixture of tert-butyl 3-(tert-butoxycarbonylamino)-3-(cyclopropylmethyl)azetidine-1-carboxylate (500 mg, 1.53 mmol, 1.0 eq) in HCl and EtOAc (10 mL, 4N) was stirred at rt for 4 h. After the reaction was completed, the precipitated solid was filtered, washed with EtOAc and concentrated to give a yellow solid (198 mg, 80% yield). LCMS m/z=127.2 (M+1).

Step 5. 3-(cyclopropylmethyl)-1-[5-[5-[(1R)-1-(3,5-dichloro-4-pyridyl)ethoxy]-1-tetrahydropyran-2-yl-indazol-3-yl]-3-fluoro-2-pyridyl]azetidin-3-amine. To a solution of 5-[(1R)-1-(3,5-dichloro-4-pyridyl)ethoxy]-3-(5,6-difluoro-3-pyridyl)-1-tetrahydropyran-2-yl-indazole (150 mg, 0.30 mmol, 1.0 eq) and 3-(cyclopropylmethyl)azetidin-3-amine HCl (75 mg, 0.59 mmol, 2.0 eq) in DMSO (5 mL) was added DIEA (383 mg, 2.97 mmol, 10.0 eq). The reaction mixture was stirred at 100° C. for 4 h, then diluted with EtOAc (150 mL) and washed with brine (100 mL×2). The organic layer was dried over Na₂SO₄ and concentrated. The crude product was purified by prep-TLC (DCM/MeOH=30/1) to give a yellow solid (120 mg, 66% yield). LCMS m/z=611.3 (M+1).

Step 6. (R)-3-(cyclopropylmethyl)-1-(5-(5-(1-(3,5-dichloropyridin-4-yl)ethoxy)-1H-indazol-3-yl)-3-fluoropyridin-2-yl)azetidin-3-amine. A mixture of 3-(cyclopropylmethyl)-1-[5-[5-[(1R)-1-(3,5-dichloro-4-pyridyl)ethoxy]-1-tetrahydropyran-2-yl-indazol-3-yl]-3-fluoro-2-pyridyl]azetidin-3-amine (110 mg, 0.18 mmol, 1.0 eq) in HCl and EtOAc (5 mL, 4M) was stirred at rt for 16 h. After the reaction completion, the reaction mixture was concentrated in vacuum. The crude product was basified with the saturate NaHCO₃ solution and extracted with EtOAc (30 mL×3). The combined organic layers were washed with brine (50 mL×2), dried over Na₂SO₄, and concentrated in vacuo. The residue was purified by Prep-HPLC (Prep-C18, 5 μM Triart column, 20×150 mm, YMC-Actus; gradient elution of 40% MeCN in water to 50% MeCN in water over a 15 min period, where both solvents contain 0.05% NH₃·H₂O) to give a white solid (45 mg, 47% yield). LCMS m/z=527.3 (M+H); 1HNMR (400 MHz, DMSO-d6) δ 13.10 (brs, 1H), 8.57 (s, 2H), 8.36 (d, J=1.6 Hz, 1H), 7.67 (dd, J=13.2 Hz, 2.0 Hz, 1H), 7.47 (d, J=9.2 Hz, 1H), 7.19 (s, 1H), 7.10 (dd, J=8.8 Hz, 1.6 Hz, 1H), 6.14 (q, J=6.4 Hz, 1H), 4.07 (d, J=8.0 Hz, 2H), 3.87 (d, J=8.0 Hz, 2H), 1.76 (d, J=6.4 Hz, 3H), 1.63 (d, J=6.4 Hz, 2H), 0.88-0.84 (m, 1H), 0.47-0.45 (m, 2H), 0.16-0.14 (m, 2H).

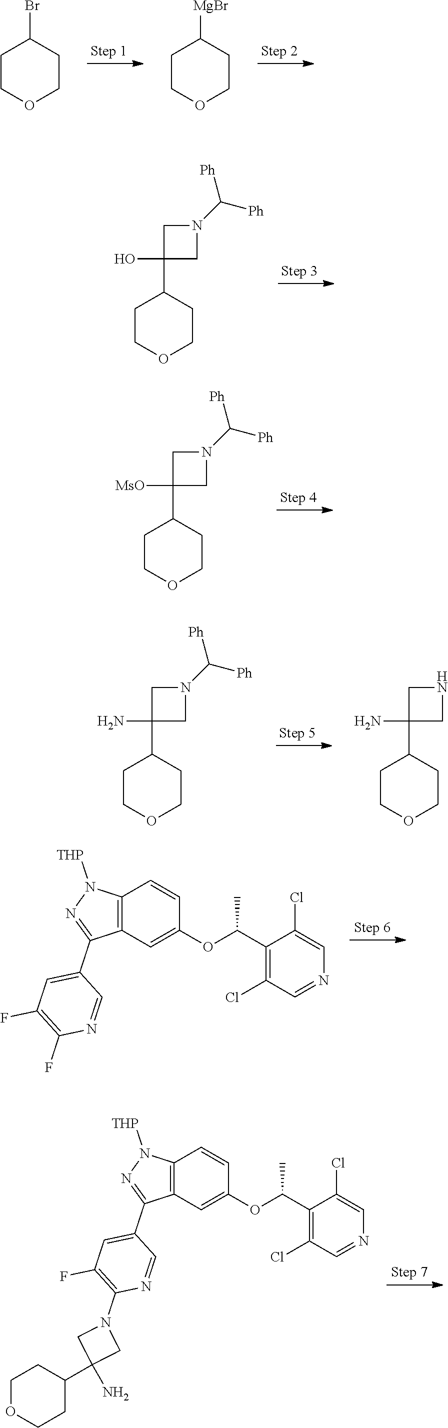

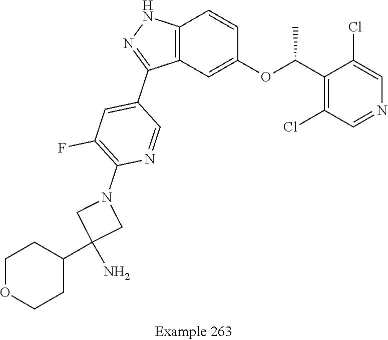 [SMILES: C[C@@H](Oc1ccc2[nH]nc(-c3cnc(N4CC(N)(C5CCOCC5)C4)c(F)c3)c2c1)c1c(Cl)cncc1Cl]

### Example 263. (R)-1-(5-(5-(1-(3,5-dichloropyridin-4-yl)ethoxy)-1H-indazol-3-yl)-3-fluoropyridin-2-yl)-3-(tetrahydro-2H-pyran-4-yl)azetidin-3-amine

Step 1. Bromo(tetrahydropyran-4-yl)magnesium. A solution of 4-bromotetrahydropyran (20.00 g, 121 mmol, 1.0 eq) in dry THE (10 mL) was added dropwise to the suspension of Mg (8.84 g, 363 mmol, 3.0 eq) and I₂ (615 mg, 2.42 mmol, 0.02 eq) in dry THE (10 mL) at 60° C. under N₂. The mixture was stirred for 2 h at 60° C. After the reaction was completed, the reaction mixture was cooled to room temperature and used directly in next step directly.

Step 2. 1-Benzhydryl-3-tetrahydropyran-4-yl-azetidin-3-ol. To a solution of 1-benzhydrylazetidin-3-one (10.00 g, 42.14 mmol, 1.0 eq) in THE (100 mL) was added bromo(tetrahydropyran-4-yl)magnesium (185 mL, 3.0 eq) at 0° C. The solution was stirred at 0° C. for 1 h under N₂ protection. After the reaction was completed, the reaction mixture was quenched with NH₄Cl solution (200 mL) and extracted with EtOAc (100 mL×3). The combined organic layers were washed with brine (100 mL), dried over Na₂SO₄, and concentrated in vacuum. The crude was triturated in MeOH (5 mL), the precipitated solid was filtered and washed with MeOH (5 mL×3) and dried under vacuum to give a white solid (6.50 g, 47.7% yield). LCMS m/z=324.2 (M+1).

Step 3. (1-Benzhydryl-3-tetrahydropyran-4-yl-azetidin-3-yl) methanesulfonate. To a solution of 1-benzhydryl-3-tetrahydropyran-4-yl-azetidin-3-ol (1.00 g, 3.09 mmol, 1.0 eq) in DCM (2 mL) were added TEA (1.25 g, 12.37 mmol, 3.0 eq) and MsCl (708 mg, 6.18 mmol, 1.5 eq) at 0° C. The reaction mixture was stirred at rt for 2 h. After the reaction was completed, the reaction mixture was poured into aqueous NaHCO₃ solution (50 mL) and extracted with EtOAc (50 mL×3). The combined organic layers were washed with brine (50 mL), dried over Na₂SO₄, and concentrated in vacuum. The crude product was purified by silica gel column (Petroleum ether/EtOAc=2:1) to give a white solid (700 mg, 56% yield). LCMS m/z=402.2 (M+1).

Step 4. 1-Benzhydryl-3-tetrahydropyran-4-yl-azetidin-3-amine. To a solution of (1-benzhydryl-3-tetrahydropyran-4-yl-azetidin-3-yl) methanesulfonate (700 mg, 1.74 mmol, 1.0 eq) in ACN (10 mL) and NH₃·H₂O (4.36 mL, 30% in water, 10.0 eq) was added K₂CO₃ (722 mg, 5.23 mmol, 3.0 eq). The mixture was stirred at 70° C. for 2 h. After the reaction was completed, the mixture was poured into water (50 mL) and extracted with EtOAc (50 mL×3). The combined organic layers were washed with brine (50 mL), dried over Na₂SO₄ and concentrated. The crude product was triturated in MTBE (5 mL) and the precipitated solid was filtered. The solid was washed with MTBE (5 mL×3) and dried under vacuum to give a white solid (440 mg, 78% yield). LCMS m/z=323.2 (M+1).

Step 5. 3-Tetrahydropyran-4-ylazetidin-3-amine. To a solution of 1-benzhydryl-3-tetrahydropyran-4-yl-azetidin-3-amine (400 mg, 1.24 mmol, 1.0 eq) in MeOH (10 mL) and conc. HCl (122 mg, 1.24 mmol, 0.1 eq) was added Pd/C (100 mg) under N₂ protection. The suspension was degassed under vacuum and purged with H2 three-times. The mixture was stirred under H₂ balloon at 25° C. for 16 h. The mixture was filtered and the filtrate was concentrated in vacuum to give) a yellow solid (430 mg, crude). LCMS m/z=157.2 (M+1).

Step 6. 1-[5-[5-[(1R)-1-(3,5-dichloro-4-pyridyl)ethoxy]-1-tetrahydropyran-2-yl-indazol-3-yl]-3-fluoro-2-pyridyl]-3-tetrahydropyran-4-yl-azetidin-3-amine. To a solution of 3-tetrahydropyran-4-ylazetidin-3-amine (93 mg, 0.59 mmol, 2.0 eq) and 5-[(1R)-1-(3,5-dichloro-4-pyridyl)ethoxy]-3-(5,6-difluoro-3-pyridyl)-1-tetrahydropyran-2-yl-indazole (150 mg, 0.29 mmol, 1.0 eq) in DMSO (1 mL) was added DIEA (383 mg, 2.97 mmol, 10.0 eq). The mixture was stirred for 4 h at 90° C., diluted with ethyl acetate (30 mL) and washed with brine (30 mL×2). The organic layer was dried over Na₂SO₄ and concentrated. The crude product was purified by silica gel column (DCM/MeOH=10/1) to give a yellow solid (140 mg, 74% yield). LCMS m/z=641.3 (M+1).

Step 7. (R)-1-(5-(5-(1-(3,5-dichloropyridin-4-yl)ethoxy)-1H-indazol-3-yl)-3-fluoropyridin-2-yl)-3-(tetrahydro-2H-pyran-4-yl)azetidin-3-amine. A solution of 1-[5-[5-[(1R)-1-(3,5-dichloro-4-pyridyl)ethoxy]-1-tetrahydropyran-2-yl-indazol-3-yl]-3-fluoro-2-pyridyl]-3-tetrahydropyran-4-yl-azetidin-3-amine (150 mg, 0.23 mmol, 1.0 eq) in TFA (1 mL) and DCM (2 mL) was stirred at 25° C. for 4 h. After the reaction was completed, the reaction mixture was treated with aqueous NaHCO₃ solution to adjust pH to 7-8 and extracted with ethyl acetate (30 mL×2). The combined organic layers were washed with brine (30 mL), dried over Na₂SO₄, and concentrated in vacuum. The residue was purified by Prep-HPLC (Prep-C18, 5 μM Triart column, 20×150 mm, YMC-Actus; gradient elution of 45% MeCN in water to 65% MeCN in water over a 15 min period, where both solvents contain 0.05% NH₄HCO₃) to give a white solid (66 mg, 50% yield). LCMS m/z=557.3 (M+H); 1HNMR (400 MHz, DMSO-d6) δ 13.14 (s, 1H), 8.57 (s, 2H), 8.39 (s, 1H), 7.74 (d, J=13.6 Hz, 1H), 7.48 (d, J=9.2 Hz, 1H), 7.19 (s, 1H), 7.11 (dd, J=8.8 Hz, 2.0 Hz, 1H), 6.14 (q, J=6.6 Hz, 1H), 4.17 (d, J=8.8 Hz, 2H), 4.02-3.92 (m, 4H), 3.33-3.26 (m, 2H), 1.98-1.92 (m, 1H), 1.76 (d, J=6.6 Hz, 3H), 1.66-1.61 (m, 2H), 1.49-1.39 (m, 2H).

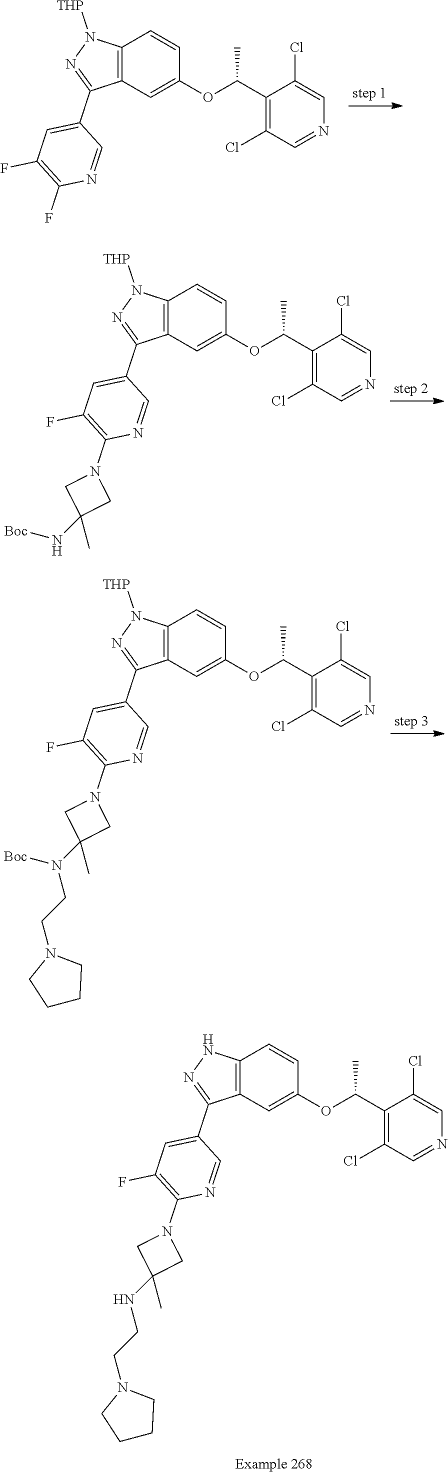

### Example 268. (R)-1-(5-(5-(1-(3,5-dichloropyridin-4-yl)ethoxy)-1H-indazol-3-yl)-3-fluoropyridin-2-yl)-3-methyl-N-(2-(pyrrolidin-1-yl)ethyl)azetidin-3-amine

Step 1. tert-butyl N-[1-[5-[5-[(1R)-1-(3,5-dichloro-4-pyridyl)ethoxy]-1-tetrahydropyran-2-yl-indazol-3-yl]-3-fluoro-2-pyridyl]-3-methyl-azetidin-3-yl]carbamate. To a solution of 5-[(1R)-1-(3,5-dichloro-4-pyridyl)ethoxy]-3-(5,6-difluoro-3-pyridyl)-1-tetrahydropyran-2-yl-indazole (350 mg, 0.69 mmol, 1.0 eq) and tert-butyl N-(3-methylazetidin-3-yl)carbamate HCl (185 mg, 0.83 mmol, 1.2 eq) in DMSO (5 mL) was added DIEA (447 mg, 3.46 mmol, 5.0 eq). The reaction mixture was stirred for 5 h at 90° C. After the reaction was completed, the mixture was diluted with Ethyl acetate (30 mL) and washed with brine (30 mL×3). The organic layer was dried over Na₂SO₄ and concentrated. The crude product was purified by silica gel chromatography eluted with Petroleum ether/Ethyl acetate (3/1) to give a light-yellow solid (300 mg, 65% yield). LCMS m/z=671.2 (M+1).

Step 2. tert-butyl N-[1-[5-[5-[(1R)-1-(3,5-dichloro-4-pyridyl)ethoxy]-1-tetrahydropyran-2-yl-indazol-3-yl]-3-fluoro-2-pyridyl]-3-methyl-azetidin-3-yl]-N-(2-pyrrolidin-1-ylethyl)carbamate. To a solution of tert-butyl N-[1-[5-[5-[(1R)-1-(3,5-dichloro-4-pyridyl)ethoxy]-1-tetrahydropyran-2-yl-indazol-3-yl]-3-fluoro-2-pyridyl]-3-methyl-azetidin-3-yl]carbamate (300 mg, 0.45 mmol, 1.0 eq) and 1-(2-chloroethyl)pyrrolidine (380 mg, 2.23 mmol, 5.0 eq) in ACN (30 mL) was added t-BuOK (300 mg, 2.68 mmol, 6.0 eq). The reaction mixture was stirred for 4 h at 50° C. After the reaction was completed, the mixture was diluted with ethyl acetate (40 mL) and washed with brine (30 mL×3). The organic layer was dried over Na₂SO₄ and concentrated. The crude product was purified by silica gel chromatography eluted with dichloromethane/methanol (10/1) to give a yellow solid (180 mg, 52% yield). LCMS m/z=768.3 (M+1).

Step 3. R)-1-(5-(5-(1-(3,5-dichloropyridin-4-yl)ethoxy)-1H-indazol-3-yl)-3-fluoropyridin-2-yl)-3-methyl-N-(2-(pyrrolidin-1-yl)ethyl)azetidin-3-amine. To a solution of tert-butyl N-[1-[5-[5-[(1R)-1-(3,5-dichloro-4-pyridyl)ethoxy]-1-tetrahydropyran-2-yl-indazol-3-yl]-3-fluoro-2-pyridyl]-3-methyl-azetidin-3-yl]-N-(2-pyrrolidin-1-ylethyl)carbamate (160 mg, 0.21 mmol, 1.0 eq) in DCM (7 mL) was added TFA (3.5 mL). The solution was stirred at rt for 5 h, then was treated with aqueous NaHCO₃ solution to adjust pH to 7-8 and extracted with DCM (40 mL×2). The combined organic layers were washed with brine (20 mL), dried over Na₂SO₄, and concentrated in vacuum. The residue was purified by Prep-HPLC (Prep-C18, 5 μM Triart column, 19×150 mm, Phenyl OBD; gradient elution of 30% MeCN in water to 60% MeCN in water over a 9 min period, where both solvents contain 10 mmoL/L NH₄HCO₃) to give a white solid (46 mg, 38% yield). LCMS m/z=584.4 (M+H); 1HNMR (400 MHz, DMSO-d6) δ 13.12 (s, 1H), 8.58 (s, 2H), 8.36 (t, J=1.6 Hz, 1H), 7.69 (dd, J=13.2 Hz, 1.6 Hz, 1H), 7.47 (d, J=9.2 Hz, 1H), 7.19 (d, J=2.0 Hz, 1H), 7.10 (dd, J=9.2 Hz, 2.4 Hz, 1H), 6.14 (q, J=6.4 Hz, 1H), 3.97 (d, J=8.4 Hz, 2H), 3.87 (d, J=8.4 Hz, 2H), 2.68-2.62 (m, 2H), 2.54-2.53 (m, 2H), 2.49-2.43 (m, 4H), 1.76 (d, J=6.4 Hz, 3H), 1.69-1.67 (m, 4H), 1.43 (s, 3H).

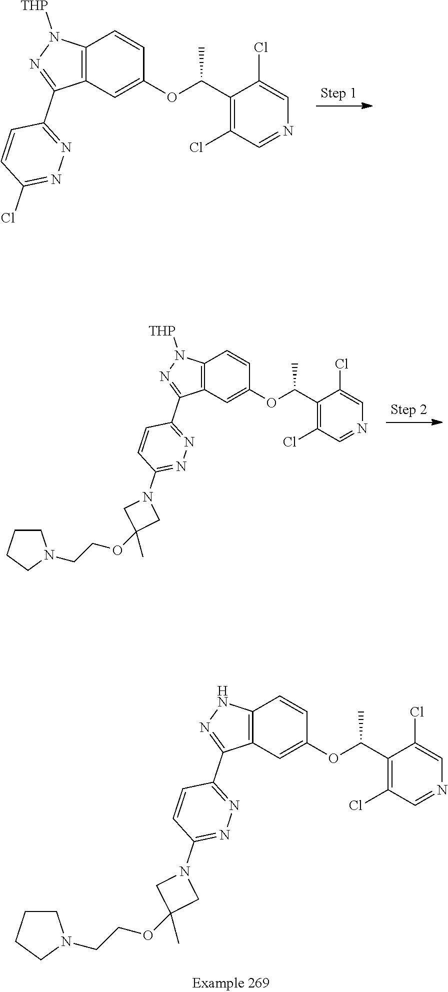

### Example 269. (R)-5-(1-(3,5-dichloropyridin-4-yl)ethoxy)-3-(6-(3-methyl-3-(2-(pyrrolidin-1-yl)ethoxy)azetidin-1-yl)pyridazin-3-yl)-1H-indazole

Step 1. 5-[(1R)-1-(3,5-dichloro-4-pyridyl)ethoxy]-3-[6-[3-methyl-3-(2-pyrrolidin-1-ylethoxy)azetidin-1-yl]pyridazin-3-yl]-1-tetrahydropyran-2-yl-indazole. To a solution of 3-(6-chloropyridazin-3-yl)-5-[(1R)-1-(3,5-dichloro-4-pyridyl)ethoxy]-1-tetrahydropyran-2-yl-indazole (120 mg, 0.24 mmol, 1.0 eq) and 1-[2-(3-methylazetidin-3-yl)oxyethyl]pyrrolidine (175 mg, 0.95 mmol, 4.0 eq) in DMSO (3 mL) was added DIEA (184 mg, 1.43 mmol, 6.0 eq). The reaction mixture was stirred for 12 h at 100° C. After the reaction was cooled to room temperature, the mixture was diluted with EtOAc (20 mL) and washed with brine (20 mL×3). The organic layer was dried over Na₂SO₄ and concentrated. The crude product was purified by Prep-TLC (DCM/MeOH=15/1) to give a yellow solid (100 mg, 65% yield). LCMS m/z=652.3 (M+1).

Step 2. (R)-5-(1-(3,5-dichloropyridin-4-yl)ethoxy)-3-(6-(3-methyl-3-(2-(pyrrolidin-1-yl)ethoxy)azetidin-1-yl)pyridazin-3-yl)-1H-indazole. To a solution of product step 1 (100 mg, 0.15 mmol, 1.0 eq) in DCM (6 mL) was added TFA (2 mL). The solution was stirred at rt for 6 h and was concentrated. The crude product was treated with DCM/MeOH (10 mL, V:V=5/1), K₂CO₃ (excess) was added and stirred for 20 minutes at rt. Then DCM (20 mL) was added. The solid was filtered out and the filtrate was concentrated. The residue was purified by Prep-TLC (DCM/MeOH=10/1) and further purified by Prep-HPLC (Prep-C18, 5 μM Triart column, 20×150 mm, YMC-Actus; gradient elution of 50% MeCN in water to 70% MeCN in water over a 9 min period, where both solvents contain 0.05% NH₃·H₂O) to give a white solid (23.0 mg, 26% yield). LCMS m/z=568.3 (M+H); 1HNMR (400 MHz, DMSO-d6) δ 13.20 (s, 1H), 8.56 (s, 2H), 7.97 (d, J=9.2 Hz, 1H), 7.94 (d, J=2.4 Hz, 1H), 7.47 (d, J=8.8 Hz, 1H), 7.09 (dd, J=9.2 Hz, 2.4 Hz, 1H), 6.91 (d, J=8.8 Hz, 1H), 6.08 (q, J=6.8 Hz, 1H), 4.06 (d, J=8.4 Hz, 2H), 3.97 (d, J=8.8 Hz, 2H), 3.53 (t, J=6.0 Hz, 2H), 2.61 (t, J=6.0 Hz, 2H), 2.49-2.45 (m, 4H), 1.77 (d, J=6.4 Hz, 3H), 1.69-1.64 (m, 4H), 1.55 (s, 3H).

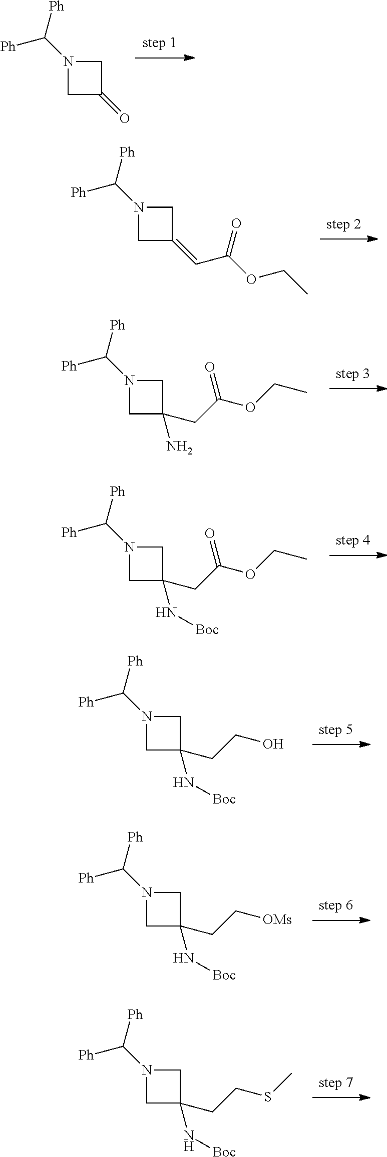

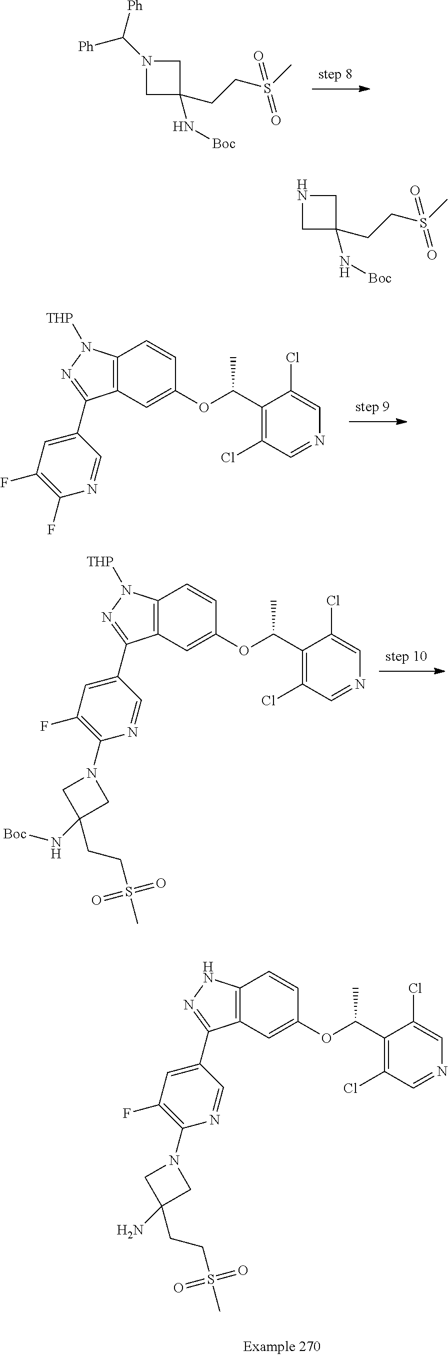

### Example 270. (R)-1-(5-(5-(1-(3,5-dichloropyridin-4-yl)ethoxy)-1H-indazol-3-yl)-3-fluoropyridin-2-yl)-3-(2-(methylsulfonyl)ethyl)azetidin-3-amine

Step 1. Ethyl 2-(1-benzhydrylazetidin-3-ylidene)acetate. To a solution of ethyl 2-diethoxyphosphoryl acetate (5.2 g, 23.2 mmol, 1.1 eq) in dry THE (50 mL) was added NaH (1.01 g, 25.3 mmol, 1.2 eq) in portions at 0° C. The mixture was stirred for 30 min, then 1-benzhydrylazetidin-3-one (5.0 g, 21.1 mmol, 1.0 eq) in dry THF (50 mL) was added. The resulting mixture was stirred for 2 h at rt. After the reaction was completed, the mixture was quenched with H₂O (100 mL), and extracted with EtOAc (100 mL×2). The combined organic layers were dried over Na₂SO₄ and concentrated under reduced pressure. The crude product was purified by silica gel chromatography (Petroleum ether/EtOAc=10/1) to give a yellow solid (6.5 g, 91% yield). LCMS m/z=308.3 (M+1).

Step 2. Ethyl 2-[3-amino-1-(1-phenylethyl)azetidin-3-yl]acetate. To a solution of ethyl 2-(1-benzhydrylazetidin-3-ylidene (3.0 g, 9.77 mmol, 1.0 eq) in EtOH (10 mL) was added NH₃·H₂O (30 mL) at rt. The resulting mixture was stirred at 60° C. for 16 h. After the reaction was completed, the mixture was cooled to rt, diluted with H₂O (100 mL), and extracted with EtOAc (100 mL×2). The combined organic layers were dried over Na₂SO₄ and concentrated to give a yellow oil (3.2 g, crude). LCMS m/z=325.3 (M+1).

Step 3. Ethyl 2-[1-benzhydryl-3-(tert-butoxycarbonylamino)azetidin-3-yl]acetate. To a solution of ethyl 2-[3-amino-1-(1-phenylethyl)azetidin-3-yl]acetate (3.2 g, crude) in DCM (50 mL) was added Boc₂O (4.3 g, 19.74 mmol, 2.0 eq) and TEA (4.1 mL, 29.61 mmol, 3.0 eq) at rt. The resulting mixture was stirred at rt for 4 h and was concentrated. The residue was purified by silica gel chromatography (Petroleum ether/THF=10/1) to give a yellow solid (3.1 g, 77% yield, two steps). LCMS m/z=425.3 (M+1).

Step 4. tert-Butyl N-[1-benzhydryl-3-(2-hydroxyethyl)azetidin-3-yl]carbamate. To a solution of ethyl 2-[1-benzhydryl-3-(tert-butoxycarbonylamino)azetidin-3-yl]acetate (3.0 g, 7.07 mmol, 1.0 eq) in THE (50 mL) was added LiAlH₄ (671 mg, 17.67 mmol, 2.5 eq) at 0° C. The resulting mixture was stirred at 0° C. for 2 h. After the reaction was completed, the mixture was quenched with H₂O at 0° C. and the solid was filtered off. The filtrate was dried over Na₂SO₄ and concentrated. The crude product was purified by silica gel chromatography (Petroleum ether/THF=3/1) to give a white solid (1.5 g, 54% yield). LCMS m/z=383.3 (M+1).

Step 5. 2-[1-Benzhydryl-3-(tert-butoxycarbonylamino)azetidin-3-yl]ethyl methanesulfonate. To a solution of tert-butyl N-[1-benzhydryl-3-(2-hydroxyethyl)azetidin-3-yl]carbamate (500 mg, 1.31 mmol, 1.0 eq) in DCM (10 mL) was added MsCl (300 mg, 2.62 mmol, 2.0 eq) and TEA (0.55 mL, 3.93 mmol, 3.0 eq) at 0° C. The resulting mixture was stirred at 0° C. for 2 h and then was quenched with ice water (10 mL) at 0° C. The aqueous layer was extracted with DCM (10 mL×2). The combined organic layers were dried over Na₂SO₄ and concentrated to give a yellow solid (600 mg, crude). LCMS m/z=461.3 (M+1).

Step 6. tert-butyl N-[1-Benzhydryl-3-(2-methylsulfanylethyl)azetidin-3-yl]carbamate. To a solution of 2-[1-Benzhydryl-3-(tert-butoxycarbonylamino)azetidin-3-yl]ethyl methanesulfonate (500 mg, crude) in DMF (10 mL) was added NaSMe (1.14 g, 3.26 mmol, 3.0 eq) at rt. The resulting mixture was stirred at 50° C. for 4 h. After the reaction was completed, the mixture was diluted with H₂O (20 mL) and extracted with EtOAc (30 mL×2). The combined organic layers were washed with brine (20 mL×2), dried over Na₂SO₄ and concentrated. The crude product was purified by silica gel chromatography (Petroleum ether/THF=5/1) to give a white solid (330 mg). LCMS m/z=413.3 (M+1).

Step 7. tert-butyl N-[1-benzhydryl-3-(2-methylsulfonylethyl)azetidin-3-yl]carbamate. To a solution of tert-butyl N-[1-benzhydryl-3-(2-methylsulfanylethyl)azetidin-3-yl]carbamate (330 mg, 0.8 mmol, 1.0 eq) in EtOH (3 mL), THF (3 mL) and H₂O (3 mL) was added Oxone (554 mg, 1.60 mmol, 2.0 eq) at rt. The resulting mixture was stirred at rt for 16 h, then diluted with H₂O (10 mL), and extracted with EtOAc (30 mL×2). The combined organic layers were dried over Na₂SO₄ and concentrated. The crude product was purified by silica gel chromatography eluted with (Petroleum ether/THF=3/1) to give a white solid (300 mg, 84% yield). LCMS m/z=445.3 (M+1).

Step 8. tert-butyl N-[3-(2-methylsulfonylethyl)azetidin-3-yl]carbamate. To a solution of tert-butyl N-[1-benzhydryl-3-(2-methylsulfonylethyl)azetidin-3-yl]carbamate (300 mg, 0.67 mmol, 1.0 eq) in MeOH (10 mL) was added Pd/C (300 mg) under N₂. The suspension was degassed under vacuum and purged with H₂ several times. The mixture was stirred under H₂ balloon at rt for 16 h. The suspension was filtered through the Celite, and the filtrate concentrated to give a yellow oil (230 mg, crude). LCMS m/z=279.2 (M+1).

Step 9. tert-butyl N-[1-[5-[5-[(1R)-1-(3,5-dichloro-4-pyridyl)ethoxy]-1-tetrahydropyran-2-yl-indazol-3-yl]-3-fluoro-2-pyridyl]-3-(2-methylsulfonylethyl)azetidin-3-yl]carbamate. To a solution of 5-[(1R)-1-(3,5-dichloro-4-pyridyl)ethoxy]-3-(5,6-difluoro-3-pyridyl)-1-tetrahydropyran-2-yl-indazole (120 mg, 0.24 mmol, 1.0 eq) and tert-butyl N-[3-(2-methylsulfonylethyl)azetidin-3-yl]carbamate (132 mg, 0.48 mmol, 2.0 eq) in DMSO (3 mL) was added DIEA (154 mg, 1.2 mmol, 5.0 eq) at rt. The resulting mixture was stirred at 100° C. for 4 h. After the reaction was completed, the reaction mixture was diluted with H₂O (10 mL) and extracted with EtOAc (20 mL×2). The combined organic layers were washed with brine (10 mL×2), dried over Na₂SO₄ and concentrated. The crude product was purified by Prep-TLC (DCM/MeOH=10/1) to give a yellow solid (140 mg, 77% yield). LCMS m/z=763.3 (M+1).

Step 10. (R)-1-(5-(5-(1-(3,5-dichloropyridin-4-yl)ethoxy)-1H-indazol-3-yl)-3-fluoropyridin-2-yl)-3-(2-(methylsulfonyl)ethyl)azetidin-3-amine. To a solution of tert-butyl N-[1-[5-[5-[(1R)-1-(3,5-dichloro-4-pyridyl)ethoxy]-1-tetrahydropyran-2-yl-indazol-3-yl]-3-fluoro-2-pyridyl]-3-(2-methylsulfonylethyl)azetidin-3-yl]carbamate (120 mg, 0.16 mmol, 1.0 eq) in DCM (4 mL) was added TFA (2 mL) at 0° C. The resulting mixture was stirred at rt for 16 h, poured into the saturated NaHCO₃ solution at 0° C., and extracted with DCM (10 mL×2). The combined organic layers were dried over Na₂SO₄ and concentrated. The crude product was purified by Prep-HPLC (Prep-C18, 5 μM Triart column, 20×150 mm, YMC-Actus; gradient elution of 40% MeCN in water to 80% MeCN in water over a 7 min period, where both solvents contain 0.05% NH₃·H₂O) to give a white solid (21 mg, 23% yield). LCMS m/z=579.4 (M+H); 1HNMR (400 MHz, DMSO-d6) δ 13.11 (s, 1H), 8.57 (s, 2H), 8.37 (t, J=1.2 Hz, 1H), 7.70 (dd, J=13.2 Hz, 2.0 Hz, 1H), 7.47 (d, J=8.8 Hz, 1H), 7.20 (d, J=2.0 Hz, 1H), 7.10 (dd, J=8.8 Hz, 2.4 Hz, 1H), 6.14 (q, J=6.8 Hz, 1H), 4.04 (dd, J=8.8 Hz, 1.6 Hz, 2H), 3.86 (d, J=7.6 Hz, 2H), 3.30-3.25 (m, 2H), 3.02 (s, 3H), 2.25 (brs, 2H), 2.18-2.11 (m, 2H), 1.76 (d, J=6.8 Hz, 3H).

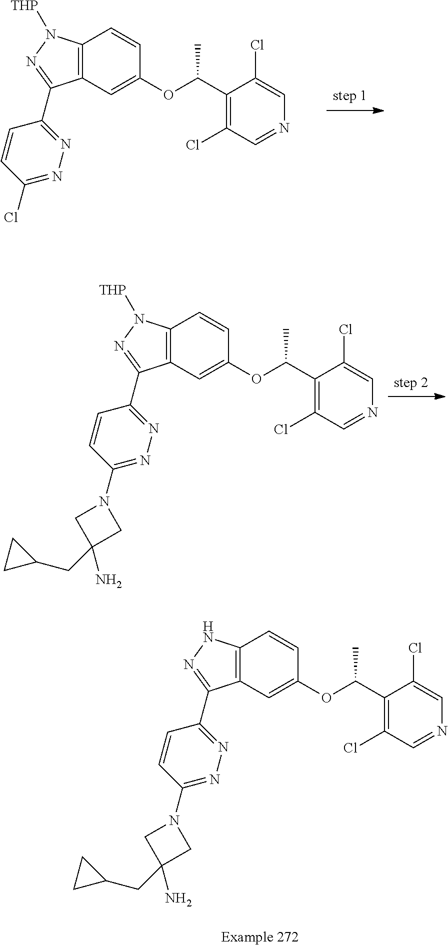

### Example 272. (R)-3-(cyclopropylmethyl)-1-(6-(5-(1-(3,5-dichloropyridin-4-yl)ethoxy)-1H-indazol-3-yl)pyridazin-3-yl)azetidin-3-amine

Step 1. 3-(cyclopropylmethyl)-1-[6-[5-[(1R)-1-(3,5-dichloro-4-pyridyl)ethoxy]-1-tetrahydropyran-2-yl-indazol-3-yl]pyridazin-3-yl]azetidin-3-amine. To a solution of 3-(6-chloropyridazin-3-yl)-5-[(1R)-1-(3,5-dichloro-4-pyridyl)ethoxy]-1-tetrahydropyran-2-yl-indazole (100 mg, 0.20 mmol, 1.0 eq) and 3-(cyclopropylmethyl)azetidin-3-amine (104 mg, 0.20 mmol, 1.0 eq) in DMSO (3 mL) was added DIEA (103.2 mg, 0.80 mmol, 4.0 eq) at room temperature. The mixture was stirred for 48 h at 110° C. After the reaction was completed, the mixture was poured into water (10 mL), and extracted with EtOAc (20 mL×3). The combined organic layers were washed with brine, dried over Na₂SO₄, and concentrated. The crude product was purified by silica gel chromatography (DCM/MeOH=20/1) to give a yellow solid (70 mg, 59% yield). LCMS m/z=594.3 (M+1).

Step 2. (R)-3-(cyclopropylmethyl)-1-(6-(5-(1-(3,5-dichloropyridin-4-yl)ethoxy)-1H-indazol-3-yl)pyridazin-3-yl)azetidin-3-amine. A solution of 3-(cyclopropylmethyl)-1-[6-[5-[(1R)-1-(3,5-dichloro-4-pyridyl)ethoxy]-1-tetrahydropyran-2-yl-indazol-3-yl]pyridazin-3-yl]azetidin-3-amine (70 mg, 0.12 mmol, 1.0 eq) in HCl/EtOAc (4M, 3 mL) was stirred for 16 h at rt. After the reaction completion, the reaction mixture was concentrated in vacuum. The crude product was treated with DCM/MeOH (15 mL, V:V=5/1), NaHCO₃ (excess) was added to the solution and stirred for 20 minutes at rt. Then DCM (20 mL) was added. The solid was filtered out and the filtrate was concentrated. The crude product was purified by Prep-TLC (DCM/MeOH=12/1) to afford the product (75 mg, ˜90% purity), which was further purified by Prep-HPLC (Prep-C18, 5 μM Triart column, 20×150 mm, YMC-Actus; gradient elution of 40% MeCN in water to 60% MeCN in water over a 10 min period, where both solvents contain 0.05% NH₃·H₂O) to give a white solid (30 mg, 50% yield). LCMS m/z=510.3 (M+H); 1HNMR (400 MHz, DMSO-d6) δ 13.13 (s, 1H), 8.52 (s, 2H), 7.93-7.91 (m, 2H), 7.43 (d, J=9.2 Hz, 1H), 7.05 (dd, J=8.8 Hz, 2.4 Hz, 1H), 6.84 (d, J=9.3 Hz, 1H), 6.04 (q, J=6.4 Hz, 1H), 4.05 (d, J=8.0 Hz, 2H), 3.80 (dd, J=8.4 Hz, 2.4 Hz, 2H), 1.73 (d, J=6.4 Hz, 3H), 1.61 (d, J=6.8 Hz, 2H), 0.87-0.81 (m, 1H), 0.45-0.41 (m, 2H), 0.14-0.10 (m, 2H).

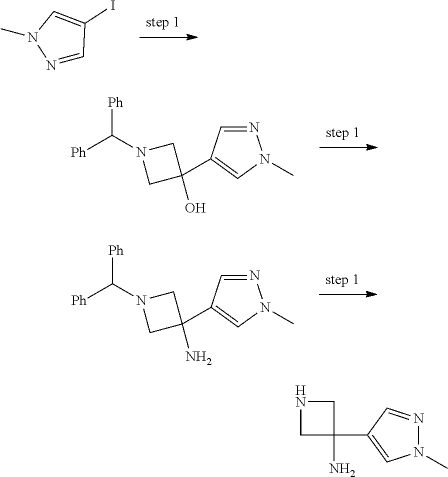

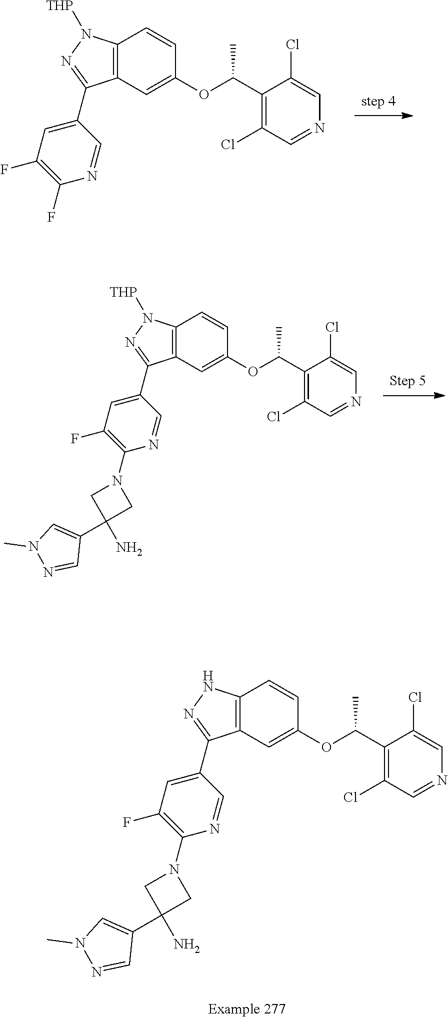

### Example 277. (R)-1-(5-(5-(1-(3,5-dichloropyridin-4-yl)ethoxy)-1H-indazol-3-yl)-3-fluoropyridin-2-yl)-3-(1-methyl-1H-pyrazol-4-yl)azetidin-3-amine

Step 1. 1-benzhydryl-3-(1-methylpyrazol-4-yl)azetidin-3-ol. To a solution of 4-iodo-1-methyl-pyrazole (1.00 g, 4.81 mmol, 1.0 eq) in THE (20 mL) was added i-PrMgBr (1 M, 5.8 mL, 5.80 mmol 1.2 eq) at 0° C. under N₂. The solution was stirred at 0-20° C. for 2 h, then 1-benzhydrylazetidin-3-one (1.14 g, 4.81 mmol, 1.0 eq) in THE (10 mL) was added to the mixture dropwise, and the solution stirred at 20° C. for 12 h. The reaction mixture was quenched with saturated aqueous NH₄Cl (30 mL) and extracted with EtOAc (30 mL×2). The combined organic layers were washed with brine (40 mL), dried over Na₂SO₄ and concentrated in vacuum. The crude was purified by silica gel column (PE/EA=1/1) to give a colorless oil (900 mg, 59% yield). LCMS m/z=320.3 (M+1).

Step 2. 1-benzhydryl-3-(1-methylpyrazol-4-yl)azetidin-3-amine. To a solution of 1-benzhydryl-3-(1-methylpyrazol-4-yl)azetidin-3-ol (850 mg, 2.66 mmol, 1.0 eq) in DCM (20 mL) was added MsCl (364 mg, 3.19 mmol, 1.2 eq) at 0° C. dropwise. The solution was stirred at 0° C. for 2 h, then the reaction mixture was added to a stirred solution of NH₃·H₂O (35 mL) and ACN (35 mL). The resulting mixture was stirred at 20° C. for 2 h then was poured into water (30 mL) and extracted with EtOAc (30 mL×2). The combined organic layers were dried over Na₂SO₄ and concentrated in vacuum. The crude product was purified by silica gel column (dichloromethane/methanol=15/1) to give a yellow oil (350 mg, 41% yield). LCMS m/z=319.3 (M+1).

Step 3. 3-(1-methylpyrazol-4-yl)azetidin-3-amine. To a solution of 1-benzhydryl-3-(1-methylpyrazol-4-yl)azetidin-3-amine (330 mg, 1.04 mmol, 1.0 eq) in MeOH (20 mL) was added Pd/C (100 mg) under N₂ protection. The suspension was degassed under vacuum and purged with H₂ 3-times. The mixture was stirred under H₂ balloon at 20° C. for 12 h, then was filtered and the filtrate concentrated in vacuum to give a colorless oil (330 mg, crude). LCMS m/z=153.2 (M+1).

Step 4. 1-[5-[5-[(1R)-1-(3,5-dichloro-4-pyridyl)ethoxy]-1-tetrahydropyran-2-yl-indazol-3-yl]-3-fluoro-2-pyridyl]-3-(1-methylpyrazol-4-yl)azetidin-3-amine. To a solution of 5-[(1R)-1-(3,5-dichloro-4-pyridyl)ethoxy]-3-(5,6-difluoro-3-pyridyl)-1-tetrahydropyran-2-yl-indazole (170 mg, 0.34 mmol, 1.0 eq) and 3-(1-methylpyrazol-4-yl)azetidin-3-amine (103 mg, crude) in DMSO (8 mL) was added DIEA (130 mg, 1.01 mmol, 3.0 eq) at room temperature. The mixture was stirred for 4 h at 100° C. After the reaction was completed, the mixture was poured into water (30 mL), which was extracted with EtOAc (30 mL×2). The combined organic layers were washed with brine (50 mL), dried over Na₂SO₄ and concentrated. The crude product was purified by Prep-TLC (Dichloromethane/Methanol=15/1) to give a yellow solid (160 mg, 75% yield). LCMS m/z=637.3 (M+1).

Step 5. (R)-1-(5-(5-(1-(3,5-dichloropyridin-4-yl)ethoxy)-1H-indazol-3-yl)-3-fluoropyridin-2-yl)-3-(1-methyl-1H-pyrazol-4-yl)azetidin-3-amine. To a solution of 1-[5-[5-[(1R)-1-(3,5-dichloro-4-pyridyl)ethoxy]-1-tetrahydropyran-2-yl-indazol-3-yl]-3-fluoro-2-pyridyl]-3-(1-methylpyrazol-4-yl)azetidin-3-amine (140 mg, 0.22 mmol, 1.0 eq) in DCM (5 mL) was added TFA (2 mL). The resulting mixture was stirred at 20° C. for 5 h. After the reaction was completed, the reaction mixture was treated with aqueous NaHCO₃ solution to adjust pH to 7-8, then was extracted with DCM (50 mL×2). The combined organic layers were washed with brine (100 mL), dried over Na₂SO₄ and concentrated in vacuum. The crude was purified by Prep-HPLC (Prep-C18, 5 μM Triart column, 20×150 mm, YMC-Actus; gradient elution of 40% MeCN in water to 60% MeCN in water over an 8 min period, where both solvents contain 0.05% NH₃·H₂O) to give a white solid (51 mg, 42% yield). LCMS m/z=553.2 (M+H); 1HNMR (400 MHz, DMSO-d6) δ 13.12 (s, 1H), 8.58 (s, 2H), 8.40 (t, J=1.6 Hz, 1H), 7.78 (s, 1H), 7.74 (dd, J=13.2 Hz, 1.6 Hz, 1H), 7.53 (s, 1H), 7.49 (d, J=9.2 Hz, 1H), 7.21 (d, J=2.0 Hz, 1H), 7.11 (dd, J=8.8 Hz, 2.0 Hz, 1H), 6.11 (q, J=6.8 Hz, 1H), 4.22 (dd, J=8.4 Hz, 2.0 Hz, 2H), 4.17 (d, J=7.6 Hz, 2H), 3.82 (s, 3H), 1.77 (d, J=6.8 Hz, 3H).

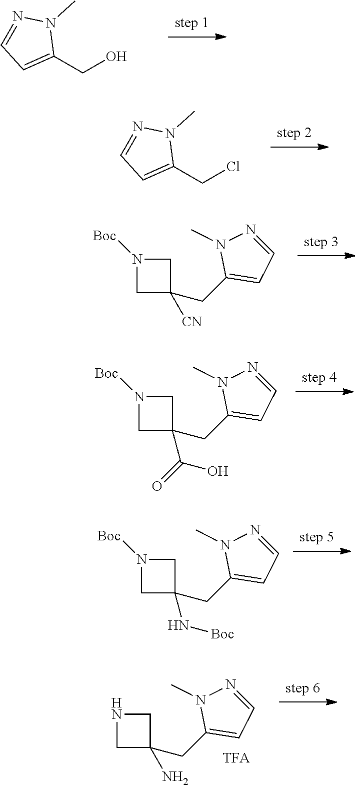

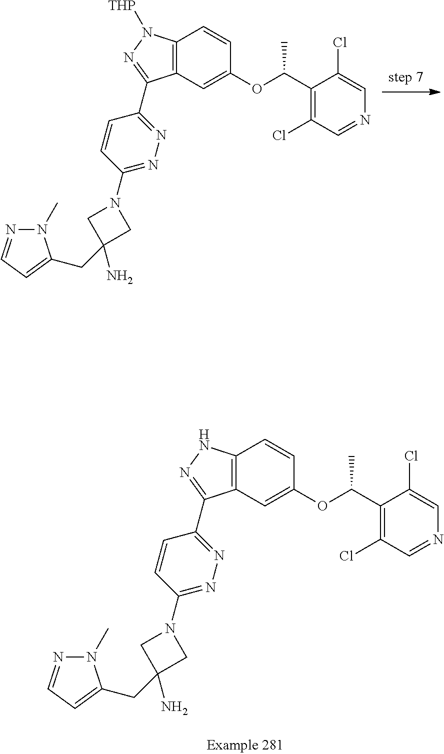

### Example 281. (R)-1-(6-(5-(1-(3,5-dichloropyridin-4-yl)ethoxy)-1H-indazol-3-yl)pyridazin-3-yl)-3-((1-methyl-1H-pyrazol-5-yl)methyl)azetidin-3-amine

Step 1. 5-(chloromethyl)-1-methyl-pyrazole. To a solution of (2-methylpyrazol-3-yl)methanol (2.0 g, 17.84 mmol, 1.0 eq) in DCM (50 mL) was added SOCl₂ (5 mL). The reaction mixture was stirred for 2 h at rt. and was concentrated. The residue was diluted with DCM (100 mL), and the pH adjusted to 7-8 with saturated NaHCO₃ solution. The organic layer was separated, dried over Na₂SO₄ and concentrated. The crude product was purified by silica gel column (Petroleum Ether/EtOAc=2/1) to give a yellow solid (1.5 g, 64% yield). LCMS m/z=131.1 (M+1).

Step 2. tert-butyl 3-cyano-3-[(2-methylpyrazol-3-yl)methyl]azetidine-1-carboxylate. To a solution of tert-butyl 3-cyanoazetidine-1-carboxylate (2.34 g, 12.87 mmol, 1.2 eq) in THE (50 mL) was added LDA (8.04 mL, 16.08 mmol, 2M in THF, 1.5 eq). The solution was stirred for 0.5 h at −70° C., then 5-(chloromethyl)-1-methyl-pyrazole (1.4 g, 10.72 mmol, 1.0 eq) in dry THE (10 mL) was added and stirred for a further 4 h at rt. The mixture was then quenched with the saturated NH₄Cl (30 mL) solution and extracted with EtOAc (50 mL×2). The organic layer was dried over Na₂SO₄ and concentrated. The crude product was purified by silica gel column (Petroleum Ether/EtOAc=2/1) to give a yellow solid (550 mg, 19% yield). LCMS m/z=277.1 (M+1).

Step 3. 1-tert-butoxycarbonyl-3-[(2-methylpyrazol-3-yl)methyl]azetidine-3-carboxylic acid. To a solution of tert-butyl 3-cyano-3-[(2-methylpyrazol-3-yl)methyl]azetidine-1-carboxylate (550 mg, 1.99 mmol, 1.0 eq) in EtOH (10 mL) and H₂O (10 mL) was added KOH (447 mg, 7.96 mmol, 4.0 eq). The reaction mixture was stirred for 3 h at 90° C. and then was concentrated. The residue was diluted with H₂O (20 mL). The pH value of the solution was adjusted to 7 with HCl aqueous (1M), and the aqueous layer was extracted with DCM/MeOH (V/V=10/1, 20 mL×3). The combined organic layers were dried over Na₂SO₄ and concentrated to give a yellow solid (330 mg, 56% yield). LCMS m/z=296.2 (M+1).

Step 4. tert-butyl 3-(tert-butoxycarbonylamino)-3-[(2-methylpyrazol-3-yl)methyl]azetidine-1-carboxylate. To a solution of 1-tert-butoxycarbonyl-3-[(2-methylpyrazol-3-yl)methyl]azetidine-3-carboxylic acid (330 mg, 1.12 mmol, 1.0 eq) in t-BuOH (10 mL) was added TEA (170 mg, 1.68 mmol, 1.5 eq) and DPPA (353 mg, 1.45 mmol, 1.3 eq). The reaction mixture was stirred for 2 h at 80° C. under N₂. After the reaction was, the mixture was diluted with H₂O (10 mL) and extracted with EtOAc (30 mL×2). The combined organic layers were dried over Na₂SO₄ and concentrated. The crude product was purified by silica gel column (Petroleum Ether/EtOAc=2/1) to give a yellow solid (240 mg, 59% yield). LCMS m/z=367.3 (M+1).

Step 5. 3-[(2-methylpyrazol-3-yl)methyl]azetidin-3-amine TFA salt. To a solution of tert-butyl 3-(tert-butoxycarbonylamino)-3-[(2-methylpyrazol-3-yl)methyl]azetidine-1-carboxylate (240 mg, 0.65 mmol, 1.0 eq) in DCM (5 mL) was added TFA (1 mL), which was stirred at rt for 6 h. The reaction mixture was then concentrated to give a yellow solid (260 mg, crude). LCMS m/z=167.3 (M+1).

Step 6. 1-[6-[5-[(1R)-1-(3,5-dichloro-4-pyridyl)ethoxy]-1-tetrahydropyran-2-yl-indazol-3-yl]pyridazin-3-yl]-3-[(2-methylpyrazol-3-yl)methyl]azetidin-3-amine. To a solution of 3-(6-chloropyridazin-3-yl)-5-[(1R)-1-(3,5-dichloro-4-pyridyl)ethoxy]-1-tetrahydropyran-2-yl-indazole (150 mg, 0.30 mmol, 1.0 eq) in DMSO (5 mL) was added 3-[(2-methylpyrazol-3-yl)methyl]azetidin-3-amine TFA salt (125 mg, 0.45 mmol, 1.5 eq) and K₂CO₃ (246 mg, 1.78 mmol, 6.0 eq). The reaction mixture was stirred for 16 h at 90° C., then was diluted with EtOAc (40 mL), washed with brine (20 mL), dried over Na₂SO₄ and concentrated. The crude product was purified by silica gel column (DCM/MeOH=20/1) to give) a yellow solid (55 mg, 29% yield. LCMS m/z=634.2 (M+1).

Step 7. (R)-1-(6-(5-(1-(3,5-dichloropyridin-4-yl)ethoxy)-1H-indazol-3-yl)pyridazin-3-yl)-3-((1-methyl-1H-pyrazol-5-yl)methyl)azetidin-3-amine. To a solution of 1-[6-[5-[(1R)-1-(3,5-dichloro-4-pyridyl)ethoxy]-1-tetrahydropyran-2-yl-indazol-3-yl]pyridazin-3-yl]-3-[(2-methylpyrazol-3-yl)methyl]azetidin-3-amine (55 mg, 0.09 mmol, 1.0 eq) in DCM (5 mL) was added TFA (1 mL). The solution was stirred at rt for 6 h, and was concentrated in vacuum. The crude product was treated with DCM/MeOH (V:V=5:1, 20 mL), NaHCO₃ solid was added and the solution stirred for 20 minutes. The solids were filtered out and the filtrate was concentrated. The crude product was purified Prep-HPLC (Prep-C18, 5 μM Triart column, 20×150 mm, YMC-Actus; gradient elution of 44% MeCN in water to 61% MeCN in water over a 8 min period, where both solvents contain 0.05% NH₃·H₂O) to give an off-white solid (13.1 mg, 28% yield). LCMS m/z=550.3 (M+H); 1HNMR (400 MHz, DMSO-d6) δ 13.17 (s, 1H), 8.56 (s, 2H), 7.97 (d, J=9.2 Hz, 1H), 7.94 (d, J=2.0 Hz, 1H), 7.47 (d, J=8.8 Hz, 1H), 7.33 (d, J=2.0 Hz, 1H), 7.09 (dd, J=8.8 Hz, 2.4 Hz, 1H), 6.91 (d, J=9.2 Hz, 1H), 6.23 (d, J=2.0 Hz, 1H), 6.08 (q, J=6.8 Hz, 1H), 4.11 (d, J=8.0 Hz, 2H), 3.86 (d, J=8.4 Hz, 2H), 3.82 (s, 3H), 3.13 (s, 2H), 1.77 (d, J=6.4 Hz, 3H).

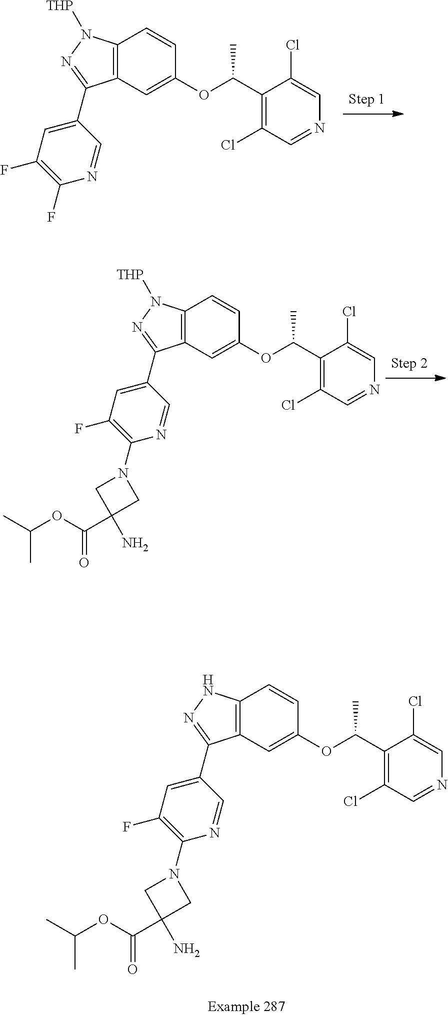

### Example 287. (R)-isopropyl 3-amino-1-(5-(5-(1-(3,5-dichloropyridin-4-yl)ethoxy)-1H-indazol-3-yl)-3-fluoropyridin-2-yl)azetidine-3-carboxylate

Step 1. isopropyl 3-amino-1-[5-[5-[(1R)-1-(3,5-dichloro-4-pyridyl)ethoxy]-1-tetrahydropyran-2-yl-indazol-3-yl]-3-fluoro-2-pyridyl]azetidine-3-carboxylate. To a solution of 5-[(1R)-1-(3,5-dichloro-4-pyridyl)ethoxy]-3-(5,6-difluoro-3-pyridyl)-1-tetrahydropyran-2-yl-indazole (200 mg, 0.40 mmol, 1.0 eq) and isopropyl 3-aminoazetidine-3-carboxylate (125 mg, 0.79 mmol, 2.0 eq) in DMSO (4 mL) was added DIEA (256 mg, 1.98 mmol, 5.0 eq). The reaction mixture was stirred for 16 h at 120° C., then was diluted with EtOAc (40 mL) and washed with brine (20 mL×3). The organic layer was dried over Na₂SO₄ and concentrated. The crude product was purified by prep-TLC (DCM/MeOH=30/1) to give a yellow solid (110 mg, 43% yield). LCMS m/z=643.3 (M+1).

Step 2. (R)-isopropyl 3-amino-1-(5-(5-(1-(3,5-dichloropyridin-4-yl)ethoxy)-1H-indazol-3-yl)-3-fluoropyridin-2-yl)azetidine-3-carboxylate To a solution of isopropyl 3-amino-1-[5-[5-[(1R)-1-(3,5-dichloro-4-pyridyl)ethoxy]-1-tetrahydropyran-2-yl-indazol-3-yl]-3-fluoro-2-pyridyl]azetidine-3-carboxylate (110 mg, 0.17 mmol, 1.0 eq) in DCM (2 mL) was added HCl/EtOAc (1 mL), the resulting mixture was stirred at rt for 4 h. After the reaction was completed, the solvent was removed, the residue was diluted with H₂O (5 mL), basified with the saturated NaHCO₃ solution, and extracted with DCM (30 mL×2). The combined organic layers were dried over Na₂SO₄ and concentrated. The crude product was purified by Prep-HPLC (Prep-C18, 5 μM Triart column, 20×150 mm, YMC-Actus; gradient elution of 30% MeCN in water to 50% MeCN in water over a 6 min period, both solvents contain 0.05% NH₃·H₂O) to give a white solid (49 mg, 51% yield). LCMS m/z=559.5 (M+H); 1HNMR (400 MHz, DMSO-d6) δ 13.13 (s, 1H), 8.58 (s, 2H), 8.39-8.39 (m, 1H), 7.74 (dd, J=12.8 Hz, 1.6 Hz, 1H), 7.48 (d, J=8.8 Hz, 1H), 7.21 (d, J=1.6 Hz, 1H), 7.10 (dd, J=9.2 Hz, 2.4 Hz, 1H), 6.15 (q, J=6.0 Hz, 1H), 5.03-4.97 (m, 1H), 4.40 (d, J=8.0 Hz, 2H), 4.02 (d, J=8.0 Hz, 2H), 1.76 (d, J=6.8 Hz, 3H), 1.25 (d, J=6.0 Hz, 6H).

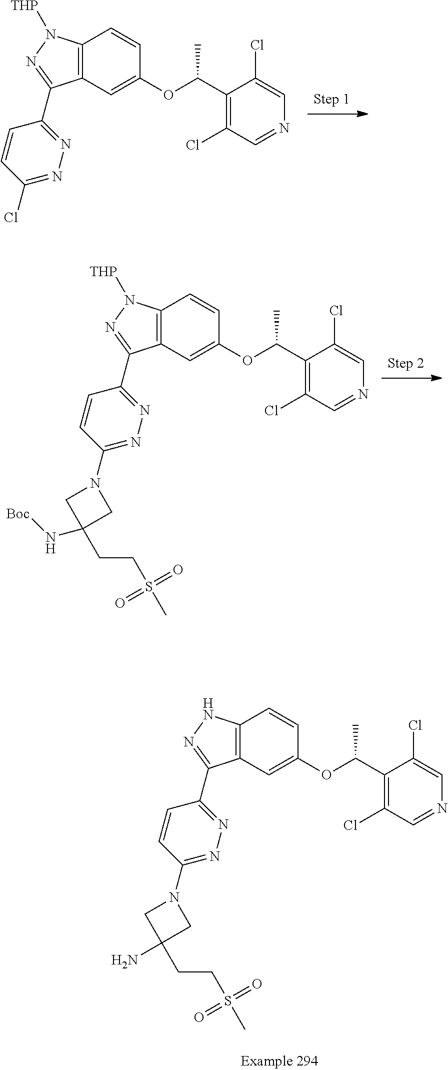

### Example 294. (R)-1-(6-(5-(1-(3,5-dichloropyridin-4-yl)ethoxy)-1H-indazol-3-yl)pyridazin-3-yl)-3-(2-(methylsulfonyl)ethyl)azetidin-3-amine

Step 1. tert-butyl N-[1-[6-[5-[(1R)-1-(3,5-Dichloro-4-pyridyl)ethoxy]-1-tetrahydropyran-2-yl-indazol-3-yl]pyridazin-3-yl]-3-(2-methylsulfonylethyl)azetidin-3-yl]carbamate. To a solution of 3-(6-chloropyridazin-3-yl)-5-[(1R)-1-(3,5-dichloro-4-pyridyl)ethoxy]-1-tetrahydropyran-2-yl-indazole (100 mg, 0.2 mmol, 1.0 eq) and tert-butyl N-[3-(2-methylsulfonylethyl)azetidin-3-yl]carbamate (111 mg, 0.4 mmol, 2.0 eq) in DMSO (3 mL) was added DIEA (77 mg, 0.6 mmol, 3.0 eq) at r.t. The resulting mixture was stirred at 100° C. for 4 h. After the reaction was completed, the reaction mixture was diluted with H₂O (10 mL) and extracted with EtOAc (10 mL×2). The combined organic layers were washed with brine (10 mL×2), dried over Na₂SO₄ and concentrated. The crude product was purified by Prep-TLC with (DCM/MeOH=10/1) to give a yellow solid (110 mg, 74% yield). LCMS m/z=746.3 (M+1).

Step 2. (R)-1-(6-(5-(1-(3,5-dichloropyridin-4-yl)ethoxy)-1H-indazol-3-yl)pyridazin-3-yl)-3-(2-(methylsulfonyl)ethyl)azetidin-3-amine. To a solution of tert-butyl N-[1-[6-[5-[(1R)-1-(3,5-Dichloro-4-pyridyl)ethoxy]-1-tetrahydropyran-2-yl-indazol-3-yl]pyridazin-3-yl]-3-(2-methylsulfonylethyl)azetidin-3-yl]carbamate (100 mg, 0.13 mmol, 1.0 eq) in DCM (4 mL) was added TFA (2 mL). The solution was stirred at rt for 16 h and then added into the mixed solvent of DCM (10 mL) and the saturated NaHCO₃ solution at 0° C. The organic layer was separated, and the aqueous phase extracted with DCM (20 mL×2). The combined organic layers were dried over Na₂SO₄ and concentrated. The crude product was purified by Prep-HPLC (Prep-C18, 5 μM Triart column, 20×150 mm, YMC-Actus; gradient elution of 50% MeCN in water to 85% MeCN in water over a 9 min period, where both solvents contain 0.05% NH₃·H2O) to give a white solid (26 mg, 34% yield). LCMS m/z=562.4 (M+H); 1HNMR (400 MHz, DMSO-d6) δ 13.19 (s, 1H), 8.56 (s, 2H), 7.97 (d, J=9.2 Hz, 1H), 7.94 (d, J=2.4 Hz, 1H), 7.47 (d, J=9.2 Hz, 1H), 7.09 (dd, J=8.8 Hz, 2.4 Hz, 1H), 6.88 (d, J=9.6 Hz, 1H), 6.08 (q, J=6.8 Hz, 1H), 4.03 (d, J=8.4 Hz, 2H), 3.81 (d, J=8.0 Hz, 2H), 3.32-3.25 (m, 2H), 3.03 (s, 3H), 2.32 (brs, 2H), 2.20-2.12 (m, 2H), 1.77 (d, J=6.8 Hz, 3H).

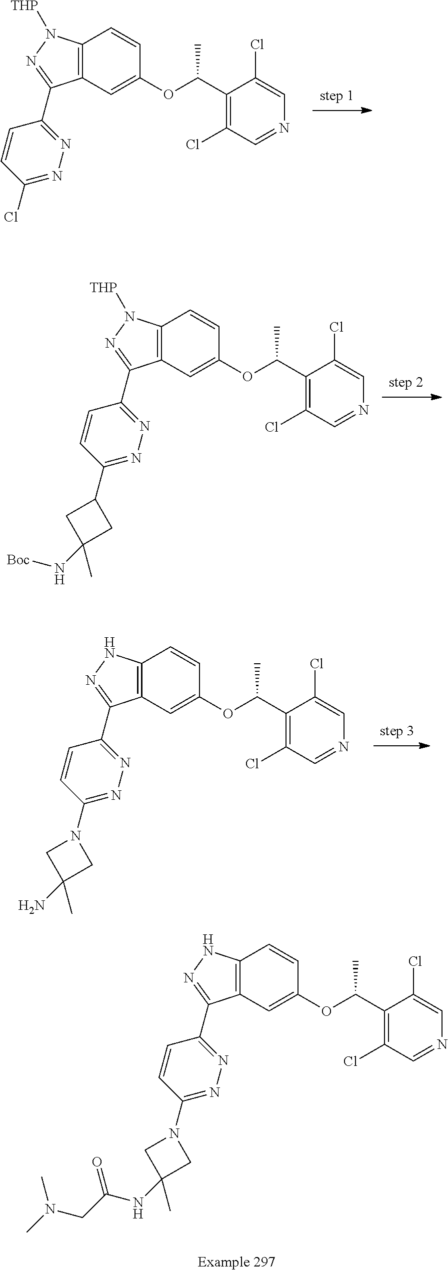

### Example 297. R)—N-(1-(6-(5-(1-(3,5-dichloropyridin-4-yl)ethoxy)-1H-indazol-3-yl)pyridazin-3-yl)-3-methylazetidin-3-yl)-2-(dimethylamino)acetamide

Step 1. tert-butyl N-[1-[6-[5-[(1R)-1-(3,5-Dichloro-4-pyridyl)ethoxy]-1-tetrahydropyran-2-yl-indazol-3-yl]pyridazin-3-yl]-3-methyl-azetidin-3-yl]carbamate. To a solution of 3-(6-chloropyridazin-3-yl)-5-[(1R)-1-(3,5-dichloro-4-pyridyl)ethoxy]-1-tetrahydropyran-2-yl-indazole (220 mg, 0.43 mmol, 1.0 eq) and tert-butyl N-(3-methylazetidin-3-yl)carbamate (291 mg, 1.31 mmol, 3.0 eq) in DMSO (6 mL) was added K₂CO₃ (601 mg, 4.36 mmol, 10.0 eq). The reaction mixture was stirred for 10 h at 90° C. After the reaction was completed, the mixture was diluted with ethyl acetate (50 mL) and washed with brine (30 mL×3). The organic layer was dried over Na₂SO₄ and concentrated. The crude product was purified by silica gel chromatography (Petroleum ether/Ethyl acetate=1/1) to give a yellow solid (220 mg, 77% yield). LCMS m/z=654.6 (M+1).

Step 2. 1-[6-[5-[(1R)-1-(3,5-Dichloro-4-pyridyl)ethoxy]-1H-indazol-3-yl]pyridazin-3-yl]-3-methyl-azetidin-3-amine. To a solution of tert-butyl N-[1-[6-[5-[(1R)-1-(3,5-Dichloro-4-pyridyl)ethoxy]-1-tetrahydropyran-2-yl-indazol-3-yl]pyridazin-3-yl]-3-methyl-azetidin-3-yl]carbamate (220 mg, 0.34 mmol, 1.0 eq) in DCM (7 mL) was added TFA (3.5 mL). The solution was stirred at rt for 6 h. After the reaction was completed, the reaction mixture was treated with NaHCO₃ aqueous solution to adjust pH to 7-8 and extracted with DCM (40 mL×2). The combined organic layers were washed with brine (30 mL×2), dried over Na₂SO₄, and concentrated in vacuum. The crude product was purified by silica gel chromatography (Dichloromethane/Methanol=10/1) to give a yellow solid (140 mg, 89% yield). LCMS m/z=470.2 (M+1).

Step 3. (R)—N-(1-(6-(5-(1-(3,5-dichloropyridin-4-yl)ethoxy)-1H-indazol-3-yl)pyridazin-3-yl)-3-methylazetidin-3-yl)-2-(dimethylamino)acetamide To a solution of 1-[6-[5-[(1R)-1-(3,5-dichloro-4-pyridyl)ethoxy]-1H-indazol-3-yl]pyridazin-3-yl]-3-methyl-azetidin-3-amine (140 mg, 0.30 mmol, 1.0 eq) and 2-(dimethylamino)acetic acid (40 mg, 0.39 mmol, 1.3 eq) in DCM (3 mL) was added T3P (284 mg, 0.89 mmol, 3.0 eq) and DIEA (192 mg, 1.49 mmol, 5.0 eq) at 0° C. The reaction mixture was stirred for 3 h at rt. After the reaction was completed, the mixture was diluted with DCM (30 mL) and washed with brine (20 mL×3). The organic layer was dried over Na₂SO₄ and concentrated. The residue was purified by Prep-TLC (dichloromethane/methanol=10/1) to give a light-yellow solid (79 mg, 48% yield). LCMS m/z=555.3 (M+H); 1HNMR (400 MHz, DMSO-d6) δ 13.26 (s, 1H), 9.00 (brs, 1H), 8.55 (s, 2H), 7.98 (d, J=9.2 Hz, 1H), 7.93 (d, J=2.0 Hz, 1H), 7.47 (d, J=8.8 Hz, 1H), 7.09 (dd, J=9.2 Hz, 2.8 Hz, 1H), 6.94 (d, J=9.6 Hz, 1H), 6.07 (q, J=6.8 Hz, 1H), 4.25 (d, J=8.4 Hz, 2H), 4.05 (d, J=8.4 Hz, 2H), 3.53 (s, 2H), 2.59 (s, 6H), 1.76 (d, J=6.8 Hz, 3H), 1.63 (s, 3H).

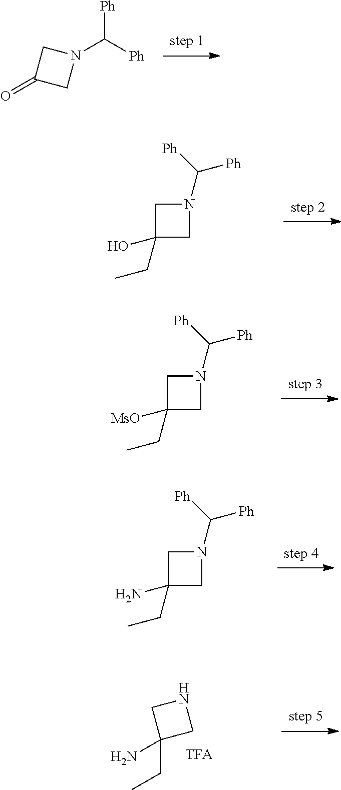

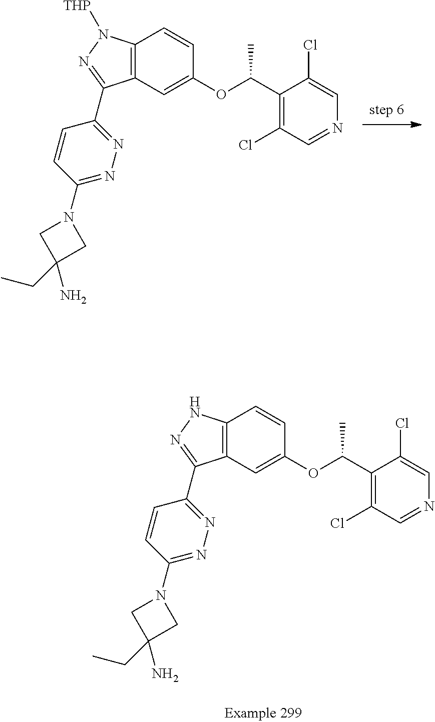

### Example 299. (R)-1-(6-(5-(1-(3,5-Dichloropyridin-4-yl)ethoxy)-1H-indazol-3-yl)pyridazin-3-yl)-3-ethylazetidin-3-amine

Step 1. 1-Benzhydryl-3-ethyl-azetidin-3-ol. To a solution of 1-benzhydrylazetidin-3-one (4.0 g, 16.86 mmol, 1.0 eq) in THF (40 mL) was added ethyl magnesium bromide (5.5 mL, 3.4 M, 18.54 mmol, 1.1 eq) dropwise at −10° C. The mixture was stirred for 2 h at −10° C. After the reaction was completed, the mixture was quenched with saturated NH₄Cl (50 mL) solution and extracted with EtOAc (80 mL×3). The combined organic layers were washed with brine, dried over Na₂SO₄ and concentrated. The crude product was purified by silica gel chromatography (Petroleum Ether/EtOAc=5/1) to give yellow oil (4.0 g, 89% yield). LCMS m/z=268.3 (M+1).

Step 2. [3-ethyl-1-(1-phenylethyl)azetidin-3-yl]methanesulfonate. To a solution of 1-benzhydryl-3-ethyl-azetidin-3-ol (4.00 g, 14.96 mmol, 1.0 eq) and TEA (3.02 g, 29.92 mmol, 2.0 eq) in DCM (50 mL) was added and MsCl (1.88 g, 16.46 mmol, 1.1 eq) at 0° C. The solution was stirred for 2 h at rt. After the reaction completion, the reaction mixture was used directly in the next step without further purification. LCMS m/z=346.3 (M+1).

Step 3. 1-Benzhydryl-3-ethyl-azetidin-3-amine. To a mixture of K₂CO₃ (24.6 g) in ACN (20 mL) and NH₃·H₂O (20 mL) was added 3-ethyl-1-(1-phenylethyl)azetidin-3-yl methanesulfonate (crude in 50 mL DCM). The mixture was stirred for 2 h at 50° C. After the reaction was completed, the reaction mixture was extracted with DCM (30 mL×3). The combined organic layers were washed with brine, dried over Na₂SO₄ and concentrated. The crude product was purified by silica gel flash column chromatography (DCM/MeOH=20/1) to give a yellow oil (2.0 g, 65% yield). LCMS m/z=267.3 (M+1).

Step 4. 3-Ethylazetidin-3-amine TFA salt. To a solution of 1-benzhydryl-3-ethyl-azetidin-3-amine (600 mg, 2.25 mmol, 1.0 eq) in i-PrOH (20 mL) was added Pd(OH)₂ (300 mg) and TFA (770 mg, 6.75 mmol, 3.0 eq) under N₂ protection. The suspension was degassed under vacuum and purged with H₂ three times, then was stirred under H₂ balloon at rt for 16 h. The solid was filtered out and the filtrate was concentrated in vacuum to give a yellow oil (650 mg, crude). LCMS m/z=101.3 (M+1).

Step 5. 1-[6-[5-[(1R)-1-(3,5-dichloro-4-pyridyl)ethoxy]-1-tetrahydropyran-2-yl-indazol-3-yl]pyridazin-3-yl]-3-ethyl-azetidin-3-amine. To a solution of 3-(6-chloropyridazin-3-yl)-5-[(1R)-1-(3,5-dichloro-4-pyridyl)ethoxy]-1-tetrahydropyran-2-yl-indazole (130 mg, 0.26 mmol, 1.0 eq) and 3-ethylazetidin-3-amine TFA salt (167.0 mg, crude) in DMSO (3 mL) was added K₂CO₃ (251.2 mg, 1.82 mmol, 7.0 eq) at room temperature. The mixture was stirred for 16 h at 90° C. After the reaction was completed, the mixture was diluted with water (10 mL) and extracted with EtOAc (30 mL×3). The combined organic layers were washed with brine, dried over Na₂SO₄ and concentrated. The crude product was purified by Prep-TLC (DCM/MeOH=15/1) to give a yellow solid (100 mg, 68% yield). LCMS m/z=568.4 (M+1).

Step 6. (R)-1-(6-(5-(1-(3,5-Dichloropyridin-4-yl)ethoxy)-1H-indazol-3-yl)pyridazin-3-yl)-3-ethylazetidin-3-amine. To a solution of 1-[6-[5-[(1R)-1-(3,5-dichloro-4-pyridyl)ethoxy]-1-tetrahydropyran-2-yl-indazol-3-yl]pyridazin-3-yl]-3-ethyl-azetidin-3-amine (100 mg, 0.17 mmol, 1.0 eq) in DCM (3 mL) was added TFA (1 mL), and stirred for 6 h at rt. After the reaction completion, the reaction mixture was concentrated in vacuum. The crude product was treated with DCM/MeOH (15 mL, V:V=5/1), and solid NaHCO₃ (excess) was added to the solution and stirred for 20 minutes at rt, followed by DCM (20 mL). The solid was filtered out and the filtrate was concentrated. The crude product was purified by Prep-TLC (DCM/MeOH=12/1) to afford the product (95 mg), which was further purified by Prep-HPLC (Prep-C18, 5 μM Triart column, 20×150 mm, YMC-Actus; gradient elution of 40% MeCN in water to 60% MeCN in water over a 10 min period, where both solvents contain 0.05% NH₃·H₂O) to give a white solid (43 mg, 50% yield). LCMS m/z=484.2 (M+H); 1HNMR (400 MHz, DMSO-d6) δ 13.16 (brs, 1H), 8.56 (s, 2H), 7.97-7.94 (m, 2H), 7.47 (d, J=8.8 Hz, 1H), 7.09 (dd, J=9.2 Hz, 2.8 Hz, 1H), 6.87 (d, J=9.2 Hz, 1H), 6.08 (q, J=6.8 Hz, 1H), 3.95 (d, J=8.0 Hz, 2H), 3.78 (d, J=8.0 Hz, 2H), 2.20 (brs, 2H), 1.78-1.70 (m, 5H), 0.97 (t, J=7.6 Hz, 3H).

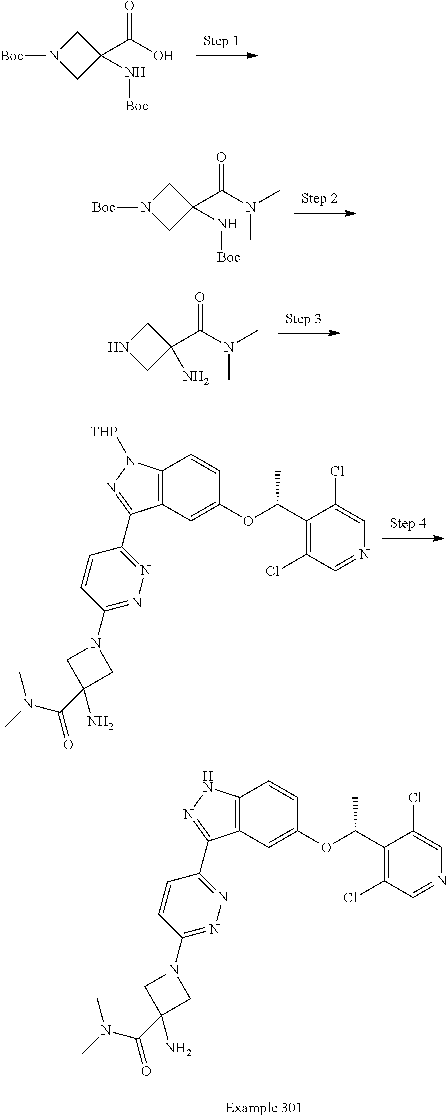

### Example 301. (R)-3-Amino-1-(6-(5-(1-(3,5-dichloropyridin-4-yl)ethoxy)-1H-indazol-3-yl)pyridazin-3-yl)-N,N-dimethylazetidine-3-carboxamide

Step 1. tert-Butyl 3-(tert-butoxycarbonylamino)-3-(dimethylcarbamoyl)azetidine-1-carboxylate. To a solution of 1-tert-butoxycarbonyl-3-(tert-butoxycarbonylamino)azetidine-3-carboxylic acid (800 mg, 2.53 mmol, 1.0 eq) in DMF (10 mL) was added HATU (1.3 g, 3.29 mmol, 1.3 eq) and DIEA (979 mg, 7.59 mmol, 3.0 eq), then dimethylamine HCl (114 mg, 2.53 mmol, 1.0 eq) was added, and the solution was stirred at rt for 16 h. After concentrating, the reaction mixture was subjected to reverse phase preparative MPLC (Prep-C18, 20-45 mM, 120 g, Tianjin Bonna-Agela Technologies; gradient elution of 20% MeCN in water to 50% MeCN in water over a 7 min period, where both solvents contain 0.05% NH₃·H₂O) to give a white solid (700 mg, 81% yield). LCMS m/z=344.1 (M+1).

Step 2. 3-Amino-N,N-dimethyl-azetidine-3-carboxamide. To a solution of tert-butyl 3-(tert-butoxycarbonylamino)-3-(dimethylcarbamoyl)azetidine-1-carboxylate (700 mg, 2.04 mmol, 1.0 eq) in EtOAc (10 mL) was added an HCl/EtOAc (10 mL), and stirred at rt for 16 h. After the reaction was completed, the mixture was concentrated. The crude product was treated with DCM/MeOH (V/V=5/1, 10 mL), solid NaHCO₃ was added, and the mixture was stirred for 20 minutes. The solid were filtered out and the filtrate was concentrated to give a white solid as the HCl salt (240 mg, crude). LCMS m/z=144.1 (M+1).

Step 3. 3-amino-1-[6-[5-[(1R)-1-(3,5-dichloro-4-pyridyl)ethoxy]-1-tetrahydropyran-2-yl-indazol-3-yl]pyridazin-3-yl]-N,N-dimethyl-azetidine-3-carboxamide. To a solution of 3-(6-chloropyridazin-3-yl)-5-[(1R)-1-(3,5-dichloro-4-pyridyl)ethoxy]-1-tetrahydropyran-2-yl-indazole (150 mg, 0.30 mmol, 1.0 eq) and 3-amino-N,N-dimethyl-azetidine-3-carboxamide (150 mg, 1.05 mmol, 3.5 eq) in DMSO (6 mL) was added DIEA (290 mg, 3.00 mmol, 3.0 eq). The reaction mixture was stirred for 16 h at 110° C. After the reaction was completed, the mixture was diluted with EtOAc (50 mL) and washed with brine (20 mL×3). The organic layer was dried over Na₂SO₄ and concentrated. The crude product was purified by prep-TLC (DCM/MeOH=20/1) to give a yellow solid (90 mg, 50% yield). LCMS m/z=611.3 (M+1).

Step 4. (R)-3-Amino-1-(6-(5-(1-(3,5-dichloropyridin-4-yl)ethoxy)-1H-indazol-3-yl)pyridazin-3-yl)-N,N-dimethylazetidine-3-carboxamide. To a solution of product step 3 (90 mg, 0.15 mmol, 1.0 eq) in EtOAc (5 mL) was added HCl/EtOAc (5 mL). The resulting mixture was stirred at rt for 16 h. After the reaction was completed, the saturated NaHCO₃ solution was added to adjust pH=7-8. The solution was extracted with EtOAc (30 mL×2). The combined organic layers were dried over Na₂SO₄ and concentrated. The crude product was purified by Pre-HPLC (Prep-C18, 5 μM Triart column, 20×150 mm, YMC-Actus; gradient elution of 40% MeCN in water to 50% MeCN in water over a 6 min period, where both solvents contain 0.05% NH₃·H₂O) to give a white solid (44.2 mg, 57% yield). LCMS m/z=527.2 (M+1); 1H NMR (400 MHz, DMSO-d6) δ 13.19 (s, 1H), 8.60 (s, 2H), 7.97 (d, J=9.2 Hz, 1H), 7.94 (d, J=2.4 Hz, 1H), 7.47 (d, J=9.2 Hz, 1H), 7.09 (dd, J=9.2 Hz, 2.4 Hz, 1H), 6.93 (d, J=9.6 Hz, 1H), 6.08 (q, J=6.8 Hz, 1H), 4.54 (d, J=8.4 Hz, 2H), 3.95 (dd, J=8.4 Hz, 3.2 Hz, 2H), 3.06 (s, 3H), 2.87 (s, 3H), 1.77 (d, J=6.4 Hz, 3H).

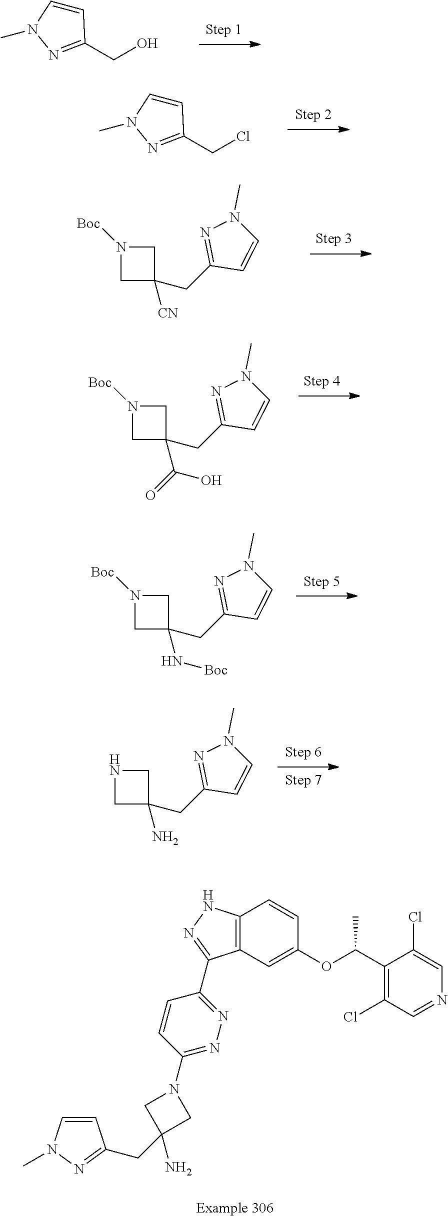

### Example 306. (R)-1-(6-(5-(1-(3,5-dichloropyridin-4-yl)ethoxy)-1H-indazol-3-yl)pyridazin-3-yl)-3-((1-methyl-1H-pyrazol-3-yl)methyl)azetidin-3-amine

Step 1. 3-(Chloromethyl)-1-methyl-pyrazole. To a solution of (1-methylpyrazol-3-yl)methanol (3.0 g, 26.75 mmol, 1.0 eq) in DCM (50 mL) was added SOCl₂ (6.4 g, 53.50 mmol, 2.0 eq) at rt. The reaction was stirred at rt for 2 h, and then was concentrated. The residue was diluted with DCM (100 ml), washed with saturated NaHCO₃ solution, and was dried over Na₂SO₄ and concentrated. The crude product was purified by silica gel chromatography (Petroleum Ether/EtOAc=5/1) to give a yellow oil (3.5 g 86% yield). LCMS m/z=131.3 (M+1).

Step 2. tert-Butyl 3-cyano-3-[(1-methylpyrazol-3-yl)methyl]azetidine-1-carboxylate. To a solution of tert-butyl 3-cyanoazetidine-1-carboxylate (4.0 g, 21.95 mmol, 1.0 eq) in dry THF (20 mL) was added LDA (16.5 ml, 2 M in THF, 32.93 mmol, 1.5 eq) at −78° C. under N₂ protection and stirred at −78° C. for 1 h. Then 3-(chloromethyl)-1-methyl-pyrazole (3.4 g, 26.34 mmol, 1.2 eq) in dry THE (5 mL) was added, and the reaction was allowed to warmed to 0° C. and stirred for 2 h. The reaction was quenched with the saturated NH₄Cl solution and extracted with EtOAc (100 ml×3). The combined organic layers were washed with brine (100 ml), dried over Na₂SO₄ and concentrated. The crude product was purified by silica gel chromatography (Petroleum Ether/EtOAc=2/1) to give a yellow oil (1.3 g 21% yield). LCMS m/z=277.3 (M+1).

Step 3. 1-tert-Butoxycarbonyl-3-[(1-methylpyrazol-3-yl)methyl]azetidine-3-carboxylic acid. To a solution of tert-butyl 3-cyano-3-[(1-methylpyrazol-3-yl)methyl]azetidine-1-carboxylate (1.8 g, 6.51 mmol, 1.0 eq) in EtOH (15 ml) and H₂O (15 ml) was added KOH (1.8 g, 32.55 mmol, 5.0 eq). The reaction was stirred at 90° C. for 6 h under N₂. After the reaction completion, the solvent was removed and the pH of the solution was adjusted to 5-6 with HCl (2M), and extracted with DCM (50 mL×3). The combined organic layers were washed with brine (20 ml), dried over Na₂SO₄, and concentrated to give a yellow oil (1.4 g, 73% yield). LCMS m/z=296.3 (M+1).

Step 4. tert-butyl 3-(tert-butoxycarbonylamino)-3-[(1-methylpyrazol-3-yl)methyl]azetidine-1-carboxylate. To a solution of 1-tert-butoxycarbonyl-3-[(1-methylpyrazol-3-yl)methyl]azetidine-3-carboxylic acid (1.4 g, 4.74 mmol, 1.0 eq) and TEA (720 mg, 7.11 mmol, 1.5 eq) in t-BuOH (20 mL) was added DPPA (1.5 g, 6.16 mmol, 1.3 eq) at rt. The reaction was stirred at 80° C. for 4 h. After the reaction completion, the reaction was diluted with EtOAc (100 ml) and washed with brine (50 ml), dried over Na₂SO₄ and concentrated. The crude product was purified by silica gel chromatography (Petroleum Ether/EtOAc=5/1) to give a white solid (1.1 g, 63% yield). LCMS m/z=367.3 (M+1).

Step 5. 3-[(1-Methylpyrazol-3-yl)methyl]azetidin-3-amine TFA salt. A solution of tert-butyl 3-(tert-butoxycarbonylamino)-3-[(1-methylpyrazol-3-yl)methyl]azetidine-1-carboxylate (1.0 g, 2.73 mmol, 1.0 eq) in DCM (5 mL) and TFA (3 ml) was stirred at rt for 4 h. After the reaction completion, the reaction was concentrated to give a yellow solid (2.3 g, crude). LCMS m/z=167.3 (M+1).

Step 6. 1-[6-[5-[(1R)-1-(3,5-dichloro-4-pyridyl)ethoxy]-1-tetrahydropyran-2-yl-indazol-3-yl]pyridazin-3-yl]-3-[(1-methylpyrazol-3-yl)methyl]azetidin-3-amine. To a solution of 3-(6-chloropyridazin-3-yl)-5-[(1R)-1-(3,5-dichloro-4-pyridyl)ethoxy]-1-tetrahydropyran-2-yl-indazole (100 mg, 0.20 mmol, 1.0 eq) and -[(1-methylpyrazol-3-yl)methyl]azetidin-3-amine TFA salt (66 mg, 0.40 mmol, 2.0 eq) in DMSO (4 ml) was added K₂CO₃ (138 mg, 1.00 mmol, 5.0 eq). The mixture was stirred at 100° C. for 16 h. After completion, the reaction was diluted with EtOAc (30 ml) and washed with brine (10 ml), dried over Na₂SO₄ and concentrated. The crude product was purified by Prep-TLC (DCM/MeOH=10/1) to give a yellow solid (40 mg, 32% yield). LCMS m/z=634.3 (M+1).

Step 7. (R)-1-[6-[5-[1-(3,5-dichloro-4-pyridyl)ethoxy]-1H-indazol-3-yl]pyridazin-3-yl]-3-[(1-methylpyrazol-3-yl)methyl]azetidin-3-amine. A solution of 1-[6-[5-[(1R)-1-(3,5-dichloro-4-pyridyl)ethoxy]-1-tetrahydropyran-2-yl-indazol-3-yl]pyridazin-3-yl]-3-[(1-methylpyrazol-3-yl)methyl]azetidin-3-amine (60 mg, 0.10 mmol, 1.0 eq) in DCM (3 mL) and TFA (1 ml) was stirred at rt for 4 h. After completion, the reaction mixture was concentrated, the residue was diluted with DCM (20 ml) and washed with the saturated NaHCO₃ solution. The organic layer was dried over Na₂SO₄ and concentrated. The crude product was purified by Prep-HPLC (Prep-C18, 5 μM Triart column, 20×150 mm, YMC-Actus; gradient elution of 30% MeCN in water to 80% MeCN in water over a 10 min period, where both solvents contain 0.05% NH₃·H₂O) to give a white solid (17 mg, 32% yield). LCMS m/z=550.2 (M+1); 1H NMR (400 MHz, DMSO-d6) δ 13.18 (s, 1H), 8.56 (s, 2H), 7.96-7.94 (m, 2H), 7.59 (d, J=2.0 Hz, 1H), 7.47 (d, J=9.2 Hz, 1H), 7.09 (dd, J=8.8 Hz, 2.4 Hz, 1H), 6.88 (d, J=9.2 Hz, 1H), 6.19 (d, J=2.0 Hz, 1H), 6.08 (q, J=6.4 Hz, 1H), 4.12 (q, J=8.0 Hz, 2H), 3.82 (q, J=6.8 Hz, 2H), 3.79 (s, 3H), 2.98 (s, 2H), 1.77 (d, J=6.4 Hz, 3H).

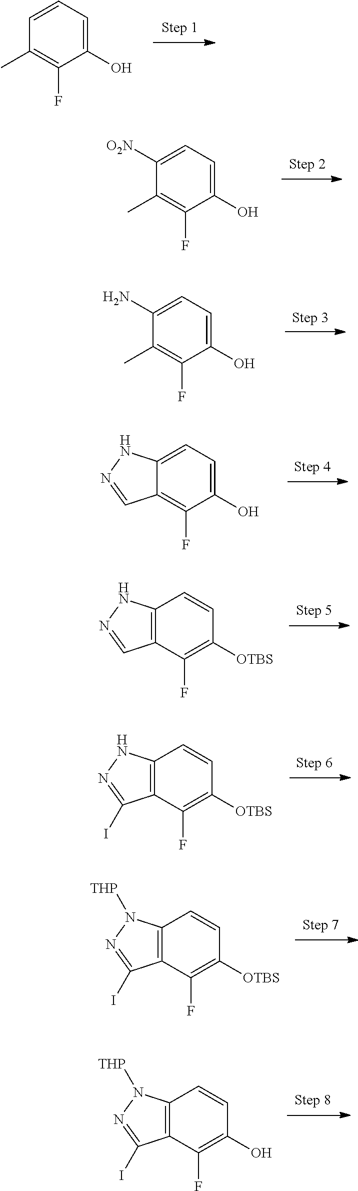

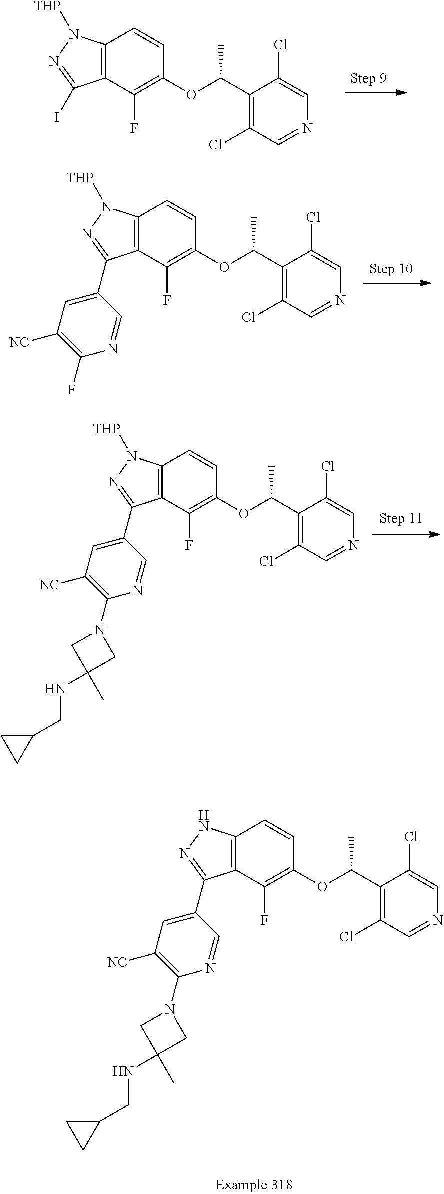

### Example 318. (R)-2-(3-((cyclopropylmethyl)amino)-3-methylazetidin-1-yl)-5-(5-(1-(3,5-dichloropyridin-4-yl)ethoxy)-4-fluoro-1H-indazol-3-yl)nicotinonitrile

Step 1. 2-Fluoro-3-methyl-4-nitro-phenol. To a solution of 2-fluoro-3-methyl-phenol (50.0 g, 394.6 mmol, 1.0 eq) and NaNO₂ (82.1 g, 1.19 mol, 3.0 eq) in H₂O (1 L) was added H₂SO₄ (300 mL) at 0° C. The reaction mixture was stirred at rt for 5 h. After the reaction was completed, the mixture was filtered, and the precipitated solid was collected and washed with water (300 mL×3). The solid was dried under vacuum to give a yellow solid (55.5 g, 82%). LCMS m/z=172.2 (M+1).

Step 2. 4-Amino-2-fluoro-3-methyl-phenol. To a solution of 2-fluoro-3-methyl-4-nitro-phenol (55.5 g, 324.3 mmol, 1.0 eq) in MeOH (1.5 L) was added Pd/C (11.1 g, 20% wt) in portions and NH₃·H₂O (568 mg, 16.22 mmol, 0.05 eq) under N₂. The suspension was degassed under vacuum and charged with hydrogen three times. The mixture was stirred at rt for 16 h under a H₂ balloon. The solid was filtered out and the filtrate was concentrated to give a yellow solid (44.4 g, 97%). LCMS m/z-142.1 (M+1).

Step 3. 4-Fluoro-1H-indazol-5-ol. A solution of 4-amino-2-fluoro-3-methyl-phenol (44.4 g, 314.6 mmol, 1.0 eq), KOAc (41.7 g, 943.7 mmol, 3.0 eq) and Ac₂O (160.6 g, 1.57 mmol, 5.0 eq) in CHCl₃ (2 L) was stirred at 0° C. for 10 min. Isoamyl nitrite (55.3 g, 471.9 mmol, 1.5 eq) was then added to the mixture at the same temperature. The reaction mixture was stirred at 80° C. for 16 h under N₂. After the mixture was cooled to rt, HCl aq. (6 M, 300 mL) and MeOH (1 L) was added, heated to 40° C. and stirred for 16 h under N₂. After the reaction was completed, the mixture was cooled to rt, and diluted with EtOAc (2 L). The aqueous phase was adjusted to pH7-8 with NaOH aqueous (1 M), the organic layer was separated, and the aqueous was extracted with EtOAc (2 L×3). The combined organic layers were dried over Na₂SO₄ and concentrated in vacuum. The crude product was purified by silica gel column (Petroleum Ether/EtOAc=6/1) to give a yellow solid (33.3 g, 70%). LCMS m/z=153.2 (M+1).

Step 4. tert-butyl-[(4-Fluoro-1H-indazol-5-yl)oxy]-dimethyl-silane. To a solution of 4-fluoro-1H-indazol-5-ol (19.00 g, 124.90 mmol, 1.0 eq) and imidazole (21.26 g, 312.24 mmol, 2.5 eq) in DMF (300 mL) was added TBSCl (28.24 g, 187.35 mmol, 1.5 eq) at 0° C. The reaction mixture was stirred at rt for 3 h. After the reaction was completed, the mixture was diluted with EtOAc (300 mL), which was washed with brine (300 mL×3). The organic layer was dried over Na₂SO₄ and concentrated in vacuum. The crude product was purified by silica gel flash column chromatography (Petroleum Ether/EtOAc=8/1) to give a purple oil (28.1 g, 85%). LCMS m/z=267.2 (M+1).

Step 5. tert-butyl-[(4-Fluoro-3-iodo-1H-indazol-5-yl)oxy]-dimethyl-silane. To a solution of tert-butyl-[(4-fluoro-1H-indazol-5-yl)oxy]-dimethyl-silane (28.10 g, 105.49 mmol, 1.0 eq) in DMF (400 mL) was added NIS (28.48 g, 126.58 mmol, 1.2 eq). The reaction mixture was stirred at rt for 3 h. After the reaction completed, the mixture was diluted with the EtOAc (500 mL) and washed with brine (200 mL×3). The organic layer was dried over Na2SO₄ and concentrated in vacuum. The crude product was purified by silica gel flash column chromatography (Petroleum Ether/EtOAc=8/1) to give a brown solid (34.8 g, 84.1%). LCMS m/z=393.3 (M+1).

Step 6. tert-butyl-(4-Fluoro-3-iodo-1-tetrahydropyran-2-yl-indazol-5-yl)oxy-dimethyl-silane. To a solution of tert-butyl-[(4-fluoro-3-iodo-1H-indazol-5-yl)oxy]-dimethyl-silane (34.80 g, 88.71 mmol, 1.0 eq) in THE (400 mL) was added DHP (14.92 g, 177.32 mmol, 2.0 eq) and TsOH (4.58 g, 26.61 mmol, 0.3 eq). The reaction mixture was stirred at 50° C. for 3 h. The reaction was cooled to room temperature and concentrated in vacuum. The crude product was purified by silica gel flash column chromatography (Petroleum Ether/EtOAc=10/1) to give orange oil (35.60 g, 96%). LCMS m/z=477.2 (M+1).

Step 7. 4-Fluoro-3-iodo-1-tetrahydropyran-2-yl-indazol-5-ol. To a solution of tert-butyl-(4-fluoro-3-iodo-1-tetrahydropyran-2-yl-indazol-5-yl)oxy-dimethyl-silane (35.60 g, 13.76 mmol, 1.0 eq) in DCM (400 mL) was added TBAF (7.82 g, 29.89 mmol, 0.4 eq). The reaction mixture was stirred at rt for 2 h. After the reaction completed, the mixture was poured into the water (500 mL) and was extracted with DCM (400 mL×2). The combined organic layers were washed with brine, dried over Na₂SO₄ and concentrated in vacuum. The crude product was purified by silica gel flash column chromatography (Petroleum Ether/EtOAc=8/1) to give an orange solid (21.4 g, 79.1%). LCMS m/z=363.1 (M+1).

Step 8. 5-[(1R)-1-(3,5-dichloro-4-pyridyl)ethoxy]-4-fluoro-3-iodo-1-tetrahydropyran-2-yl-indazole. A solution of 4-fluoro-3-iodo-1-tetrahydropyran-2-yl-indazol-5-ol (10.00 g, 27.61 mmol, 1.0 eq), (1S)-1-(3,5-dichloro-4-pyridyl)ethanol (5.30 g, 27.61 mmol, 1.0 eq) and PPh₃ (14.49 g, 55.23 mmol, 2.0 eq) in DCM (200 mL) was stirred at 0° C. for 10 min. Then, DIAD (8.38 g, 41.42 mmol, 1.5 eq) in DCM (20 mL) was added at the same temperature, and the mixture stirred at rt for 3 h. The reaction was concentrated in vacuum and the crude product purified by silica gel flash column chromatography (Petroleum Ether/EtOAc=10/1) to give an orange solid (10.8 g, 73% yield). LCMS m/z=536.3 (M+1); 1H NMR (400 MHz, DMSO-d6) δ 8.61 (s, 2H), 7.49 (d, J=9.2 Hz, 1H), 7.21-7.16 (m, 1H), 6.02 (q, J=6.8 Hz, 1H), 5.76-5.72 (m, 1H), 3.81-3.78 (m, 1H), 3.72-3.64 (m, 1H), 2.34-2.26 (m, 1H), 1.99-1.90 (m, 2H), 1.79 (d, J=6.0 Hz, 3H), 1.74-1.62 (m, 1H), 1.59-1.48 (m, 2H).

Step 9. 5-[5-[(1R)-1-(3,5-dichloro-4-pyridyl)ethoxy]-4-fluoro-1-tetrahydropyran-2-yl-indazol-3-yl]-2-fluoro-pyridine-3-carbonitrile. To a solution of 5-[(1R)-1-(3,5-dichloro-4-pyridyl)ethoxy]-4-fluoro-3-iodo-1-tetrahydropyran-2-yl-indazole. A solution of 4-fluoro-3-iodo-1-tetrahydropyran-2-yl-indazol-5-ol (3.60 g, 6.71 mmol, 1.0 eq) and 2-fluoro-5-(4,4,5,5-tetramethyl-1,3,2-dioxaborolan-2-yl)pyridine-3-carbonitrile (5.0 g, 20.14 mmol, 3.0 eq) in dioxane (40 mL) and water (4 mL) was added K₂CO₃ (2.78 g, 20.14 mmol, 3.0 eq) and Pd(dppf)Cl₂ (31 mg, 0.038 mmol, 0.1 eq). The reaction mixture was stirred at 95° C. for 3 h under N2. After the reaction was completed, the solid was filtered out and the filtrate was concentrated in vacuum. The crude product was purified by silica gel flash column chromatography (Petroleum Ether/EtOAc=4/1) to give orange solid (2.8 g, 79%). LCMS m/z=512.1 (M+1).

Step 10. 2-[3-(cyclopropylmethylamino)-3-methyl-azetidin-1-yl]-5-[5-[(1R)-1-(3,5-dichloro-4-pyridyl)ethoxy]-4-fluoro-1-tetrahydropyran-2-yl-indazol-3-yl]pyridine-3-carbonitrile. To a solution of 5-[5-[(1R)-1-(3,5-dichloro-4-pyridyl)ethoxy]-4-fluoro-1-tetrahydropyran-2-yl-indazol-3-yl]-2-fluoro-pyridine-3-carbonitrile (150 mg, 0.28 mmol, 1.0 eq) and N-(cyclopropylmethyl)-3-methyl-azetidin-3-amine (59 mg, 0.42 mol, 1.5 eq) in DMSO (2 mL) was added DIEA (109 mg, 0.85 mmol, 3.0 eq). The reaction mixture was stirred at 60° C. for 3 h. After the reaction was completed, the mixture was diluted with EtOAc (10 mL) and washed with brine (10 mL×3). The organic layer was dried over Na₂SO₄ and concentrated in vacuum. The crude product was purified by prep-TLC (DCM/MeOH=30/1) to give a white solid (150 mg, 82%). LCMS m/z=650.1 (M+1).

Step 11. (R)-2-(3-((cyclopropylmethyl)amino)-3-methylazetidin-1-yl)-5-(5-(1-(3,5-dichloropyridin-4-yl)ethoxy)-4-fluoro-1H-indazol-3-yl)nicotinonitrile. To a solution of 2-[3-(cyclopropylmethylamino)-3-methyl-azetidin-1-yl]-5-[5-[(1R)-1-(3,5-dichloro-4-pyridyl)ethoxy]-4-fluoro-1-tetrahydropyran-2-yl-indazol-3-yl]pyridine-3-carbonitrile (150 mg, 0.24 mmol, 1.0 eq) in DCM (3 mL) was added TFA (1 mL). The reaction mixture was stirred at rt for 3 h. After the reaction was completed, the reaction mixture was concentrated. The crude product was treated with NaHCO₃ (aqueous) to adjust pH to 7-8, and extracted with DCM (20 mL×3). The combined organic layers were washed with brine, dried over Na₂SO₄, and concentrated. The crude product was purified by Pre-TLC (DCM/MeOH=15/1) to give a white solid (42 mg, 32%). LCMS m/z=566.2 (M+H); 1HNMR (400 MHz, DMSO-d6) δ 13.50 (s, 1H), 8.69 (s, 1H), 8.61 (s, 2H), 8.21 (s, 1H), 7.28 (d, J=9.2 Hz, 1H), 7.15-7.11 (m, 1H), 5.99 (q, J=6.8 Hz, 1H), 4.20-4.14 (m, 2H), 4.05-4.00 (m, 2H), 2.47-2.39 (m, 2H), 1.79 (d, J=6.4 Hz, 3H), 1.43 (s, 3H), 0.92-0.81 (m, 1H), 0.46-0.42 (m, 2H), 0.20-0.13 (m, 2H).

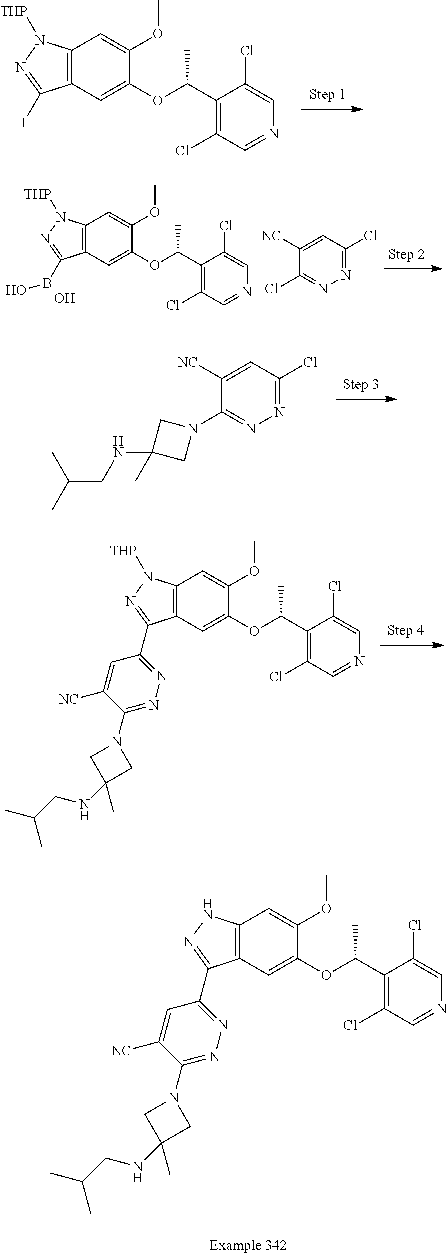

### Example 342. R)-6-(5-(1-(3,5-dichloropyridin-4-yl)ethoxy)-6-methoxy-1H-indazol-3-yl)-3-(3-(isobutylamino)-3-methylazetidin-1-yl)pyridazine-4-carbonitrile

Step 1. [5-[(1R)-1-(3,5-dichloro-4-pyridyl)ethoxy]-6-methoxy-1-tetrahydropyran-2-yl-indazol-3-yl]boronic acid. A mixture of 5-[(1R)-1-(3,5-dichloro-4-pyridyl)ethoxy]-3-iodo-6-methoxy-1-tetrahydropyran-2-yl-indazole (2.0 g, 3.65 mmol, 1.0 eq), BPD (1.85 g, 7.30 mmol, 2.0 eq), KOAc (715 mg, 7.30 mmol, 2.0 eq) and Pd(dppf)Cl₂ (267 mg, 0.37 mmol, 0.1 eq) in DMSO (15 ml) was stirred at 100° C. for 16 h under N₂. The reaction was then diluted with EtOAc (100 ml) and washed with brine (50 ml). The organic layer was dried over Na₂SO₄ and concentrated. The crude product was purified by silica gel chromatography (DCM/MeOH=25/1) to give a yellow solid (1.0 g 59% yield). LCMS m/z=466.3 (M+1).

Step 2. 6-chloro-3-[3-(isobutylamino)-3-methyl-azetidin-1-yl]pyridazine-4-carbonitrile. To a solution of 3,6-dichloropyridazine-4-carbonitrile (500 mg, 2.87 mmol, 1.0 eq) and N-isobutyl-3-methyl-azetidin-3-amine (815 mg, 5.74 mmol, 2.0 eq) in DMSO (15 ml) was added DIEA (1.9 g, 14.35 mmol, 5.0 eq). The solution was stirred at 100° C. for 16 h. The reaction was diluted with EtOAc (100 ml) and washed with brine (50 ml). The organic layer was dried over Na₂SO₄ and concentrated. The crude product was purified by silica gel chromatography (DCM/MeOH=25/1) to give a yellow solid (460 mg 57% yield). LCMS m/z=280.2 (M+1).

Step 3. 6-[5-[(1R)-1-(3,5-dichloro-4-pyridyl)ethoxy]-6-methoxy-1-tetrahydropyran-2-yl-indazol-3-yl]-3-[3-(isobutylamino)-3-methyl-azetidin-1-yl]pyridazine-4-carbonitrile. To a solution of 6-chloro-3-[3-(isobutylamino)-3-methyl-azetidin-1-yl]pyridazine-4-carbonitrile (120 mg, 0.43 mmol, 1.0 eq) and [5-[(1R)-1-(3,5-dichloro-4-pyridyl)ethoxy]-6-methoxy-1-tetrahydropyran-2-yl-indazol-3-yl]boronic acid (260 mg, 0.56 mmol, 1.3 eq) in dioxane (10 ml) and H2a (1 ml) was added K₂CO₃ (118 mg, 0.86 mmol, 2.0 eq) and Pd(dppf)Cl₂ (31 mg, 0.04 mmol, 0.1 eq). The reaction mixture was stirred at 100° C. for 16 h under N₂. The mixture was diluted with EtOAc (100 ml) and washed with brine (50 ml). The organic layer was dried over Na₂SO₄ and concentrated. The crude product was purified by Prep-TLC (DCM/MeOH=20/1) to give a yellow solid (110 mg, 39% yield). LCMS m/z=665.4 (M+1).

Step 4. (R)-6-(5-(1-(3,5-dichloropyridin-4-yl)ethoxy)-6-methoxy-1H-indazol-3-yl)-3-(3-(isobutylamino)-3-methylazetidin-1-yl)pyridazine-4-carbonitrile. A solution of 6-[5-[(1R)-1-(3,5-dichloro-4-pyridyl)ethoxy]-6-methoxy-1-tetrahydropyran-2-yl-indazol-3-yl]-3-[3-(isobutylamino)-3-methyl-azetidin-1-yl]pyridazine-4-carbonitrile (110 mg, 0.17 mmol, 1.0 eq) in DCM (5 mL) and TFA (2 mL) was stirred at rt for 4 h. After the reaction was completed, the reaction mixture was concentrated, and the residue was diluted with DCM (20 ml) and washed with the saturated NaHCO₃ solution. The organic layer was dried over Na₂SO₄ and concentrated. The crude product was purified by Prep-HPLC (Prep-C18, 5 μM Triart column, 20×150 mm, YMC-Actus; gradient elution of 30% MeCN in water to 70% MeCN in water over a 12 min period, where both solvents contain 0.05% NH₃·H₂O) to give a yellow solid (44 mg, 46% yield). LCMS m/z=581.3 (M+H); 1HNMR (400 MHz, DMSO-d6) δ 13.18 (s, 1H), 8.57 (s, 2H), 8.26 (s, 1H), 7.69 (s, 1H), 7.03 (s, 1H), 5.91 (q, J=6.4 Hz, 1H), 4.23 (d, J=8.8 Hz, 2H), 4.13 (d, J=8.8 Hz, 2H), 3.87 (s, 3H), 2.36 (d, J=6.0 Hz, 2H), 1.75 (d, J=6.8 Hz, 3H), 1.63-1.60 (m, 1H), 1.45 (s, 3H), 0.92 (d, J=6.4 Hz, 6H).

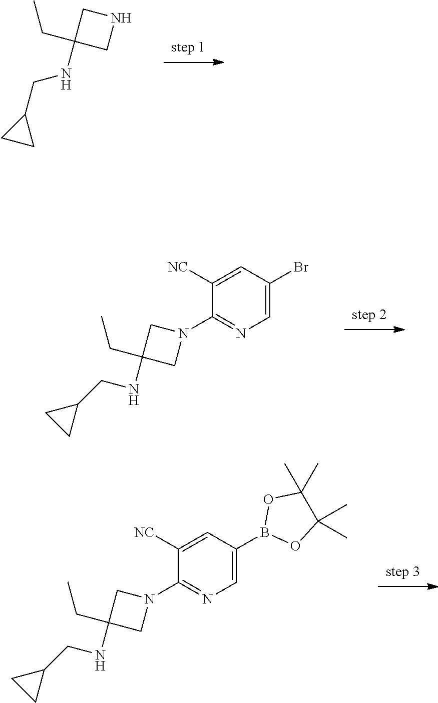 [SMILES: CCC1(NCC2CC2)CNC1 ; CCC1(NCC2CC2)CN(c2ncc(Br)cc2C#N)C1 ; CCC1(NCC2CC2)CN(c2ncc(B3OC(C)(C)C(C)(C)O3)cc2C#N)C1]

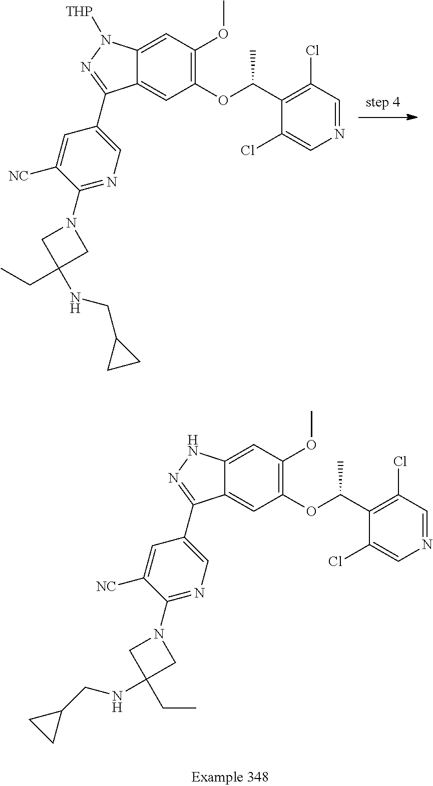

### Example 348

Step 1. 5-Bromo-2-(3-((cyclopropylmethyl)amino)-3-ethylazetidin-1-yl)nicotinonitrile. A mixture of 5-bromo-2-chloro-pyridine-3-carbonitrile (453 mg, 2.08 mmol, 1 equiv), N-(cyclopropylmethyl)-3-ethyl-azetidin-3-amine 2HCl (473 mg, 2.08 mmol, 1 equiv) and N,N-diisopropylethylamine (3.6 mL, 20.8 mmol, 10 equiv) in acetonitrile (40 mL) was heated at 60° C. for 2 hours. The reaction was cooled to room temperature and diluted with ethyl acetate (200 mL) and saturated sodium carbonate solution (50 mL). The layers were separated, and the organic layer was washed with saturated brine (2×30 mL), dried over sodium sulfate, filtered and concentrated under reduced pressure. The residue was purified twice on a Biotage automated chromatography system (Biotage Sfar KP-Amino D Duo 50 μm 28 g then a 55 g column), eluting with a gradient of 0 to 50% ethyl acetate in hexanes to give an off-white solid (560 mg, 80% yield). LCMS m/z=335.1 (M+H).

Step 2. 2-(3-((Cyclopropylmethyl)amino)-3-ethylazetidin-1-yl)-5-(4,4,5,5-tetramethyl-1,3,2-dioxaborolan-2-yl)nicotinonitrile. A mixture of 5-bromo-2-(3-((cyclopropylmethyl)amino)-3-ethylazetidin-1-yl)nicotinonitrile (168 mg, 0.5 mmol, 1 equiv), bis(pinacolato)diboron (140 mg, 0.55 mmol, 1.1 equiv), potassium acetate (59 mg, 0.6 mmol, 1.2 equiv) and [1,1′-bis(diphenylphosphino)ferrocene]dichloropalladium (II) dichloromethane complex (29 mg, 0.035 mmol, 0.07 equiv) in 1,4-dioxane (10 mL) was sparged with nitrogen for 10 minutes, and then heated at 80° C. for 16 hours. LCMS analysis indicated that the reaction was completed. The crude reaction mixture was cooled to room temperature and used subsequently in the next step. LCMS m/z=301.2 (boronic acid) and 383.3 (M+H).

Step 3. 2-(3-((Cyclopropylmethyl)amino)-3-ethylazetidin-1-yl)-5-(5-((R)-1-(3,5-dichloropyridin-4-yl)ethoxy)-6-methoxy-1-(tetrahydro-2H-pyran-2-yl)-1H-indazol-3-yl)nicotinonitrile. The mixture from step 2 (0.5 mmol, 1 equiv) in 1,4-dioxane was treated with 5-[(1R)-1-(3,5-dichloro-4-pyridyl)ethoxy]-3-iodo-6-methoxy-1-tetrahydropyran-2-yl-indazole (274 mg, 0.5 mmol, 1 equiv), [1,1′-bis(diphenylphosphino)ferrocene]dichloropalladium (II) dichloromethane complex (29 mg, 0.035 mmol, 0.07 equiv), potassium carbonate (138 mg, 1 mmol, 2 equiv) and water (1.0 mL). After sparging with nitrogen for 10 minutes, the reaction was heated at 100° C. for 16 hours. The mixture was cooled to room temperature, and filtered through a pad of Celite, which was washed with tetrahydrofuran (30 mL). The filtrate was concentrated under reduced pressure. The residue was purified on a Biotage automated chromatography system (Biotage Sfar KP-Amino D Duo 50 μm 28 g column), eluting with a gradient of 0 to 100% ethyl acetate in hexanes. Product containing fractions were combined and concentrated. The residue was dissolved in dimethyl sulfoxide (8 mL) and purified on a Biotage automated chromatography system (RediSep Rf GOLD 100 g HP C18 column), eluting with a gradient of 0 to 100% acetonitrile in water to give an off-white solid (210 mg, 62% yield). LCMS m/z=676.3 (M+H).

Step 4. (R)-2-(3-((Cyclopropylmethyl)amino)-3-ethylazetidin-1-yl)-5-(5-(1-(3,5-dichloropyridin-4-yl)ethoxy)-6-methoxy-1H-indazol-3-yl)nicotinonitrile dihydrochloride. Product step 3 (190 mg, 0.28 mmol, 1.0 equiv) was treated with 2M HCl (1.1 mL, 2.25 mmol, 8 equiv) in ethanol (4.2 mL) at 65° C. After 6 hours, the mixture was cooled to room temperature. Methyl tert-butyl ether (15 mL) was added dropwise, and the mixture was vigorously stirred for 20 minutes. The resulting solids were filtered, washed with methyl tert-butyl ether (10 mL) and dried at 40° C. under vacuum for 70 hours to give a yellow solid as the 2HCl salt (144 mg, 70%). LCMS m/z=592.2 (M+H). free base; 1H NMR (400 MHz, DMSO-d6) δ=13.08 (br s, 1H), 9.84 (br s, 2H), 8.77 (d, J=2.3 Hz, 1H), 8.59 (s, 2H), 8.17 (d, J=2.3 Hz, 1H), 7.06 (s, 1H), 7.02 (s, 1H), 6.00 (q, J=6.6 Hz, 1H), 4.54 (d, J=10.0 Hz, 2H), 4.29 (d, J=10.0 Hz, 2H), 3.86 (s, 3H), 2.95-2.81 (m, 2H), 2.00 (q, J=7.3 Hz, 2H), 1.76 (d, J=6.6 Hz, 3H), 1.18-1.10 (m, 1H), 1.06 (t, J=7.4 Hz, 3H), 0.65-0.55 (m, 2H), 0.47-0.39 (m, 2H).

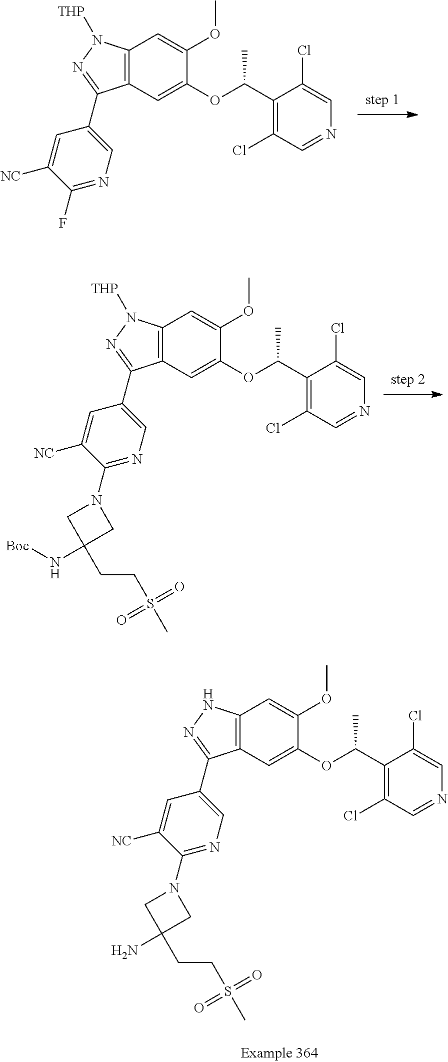

### Example 364. (R)-2-(3-amino-3-(2-(methylsulfonyl)ethyl)azetidin-1-yl)-5-(5-(1-(3,5-dichloropyridin-4-yl)ethoxy)-6-methoxy-1H-indazol-3-yl)nicotinonitrile

Step 1. tert-butyl N-[1-[3-cyano-5-[5-[(1R)-1-(3,5-dichloro-4-pyridyl)ethoxy]-6-methoxy-1-tetrahydropyran-2-yl-indazol-3-yl]-2-pyridyl]-3-(2-methylsulfonylethyl)azetidin-3-yl]carbamate. To a solution of 5-[5-[(1R)-1-(3,5-dichloro-4-pyridyl)ethoxy]-6-methoxy-1-tetrahydropyran-2-yl-indazol-3-yl]-2-fluoro-pyridine-3-carbonitrile (120 mg, 0.22 mmol, 1.0 eq) and tert-butyl N-[3-(2-methylsulfonylethyl)azetidin-3-yl]carbamate (123 mg, 0.44 mmol, 2.0 eq) in DMSO (3 mL) was added DIEA (171 mg, 1.33 mmol, 6.0 eq). The reaction mixture was stirred for 12 h at 100° C. After cooled to room temperature, the mixture was diluted with EtOAc (20 mL) and washed with brine (20 mL×3). The organic layer was dried over Na₂SO₄ and concentrated. The crude product was purified by prep-TLC (DCM/MeOH=20/1) to give a yellow solid (100 mg, 57% yield). LCMS m/z=800.3 (M+1).

Step 2: (R)-2-(3-amino-3-(2-(methylsulfonyl)ethyl)azetidin-1-yl)-5-(5-(1-(3,5-dichloropyridin-4-yl)ethoxy)-6-methoxy-1H-indazol-3-yl)nicotinonitrile. To a solution of Example 1521c (100 mg, 0.12 mmol) in DCM (6 mL) was added TFA (2 mL) and stirred at rt for 6 h. After the reaction was completed, the reaction mixture was concentrated. The crude product was treated with DCM/MeOH (10 mL, V:V=5/1), K₂CO₃ (excess) was added to the solution and stirred for 20 minutes at rt, then DCM (20 mL) was added. The solid was filtered out and the filtrate was concentrated, the residue was purified by Prep-TLC (DCM/MeOH=10/1) and further purified by Prep-HPLC (Prep-C18, 5 M Triart column, 20×150 mm, YMC-Actus; gradient elution of 50 MeCN in water to 703 MeCN in water over a 9 min period, where both solvents contain 0.0500 NHl₃·H₂O) to give a white solid (49 mg, 64% yield). LCMS m/z=616.3 (M+H); 1HNMR (400 Mz, DMSO-d6) δ 12.98 (brs, 1H), 8.72 (d, J=2.4 Hz, 1H), 8.59 (s, 2H), 8.06 (d, J=2.0 Hz, 1H), 7.03 (s, 1H), 7.01 (s, 1H), 5.99 (q, J=6.4 Hz, 1H), 4.20 (d, J=8.8 Hz, 2H), 4.00 (d, J=8.8 Hz, 2H), 3.86 (s, 3H), 3.31-3.23 (i, 2H), 3.02 (s, 3H), 2.42 (brs, 2H), 2.18-2.10 (i, 2H), 1.76 (d, J=6.8 Hz, 3H)

TABLE
Examples synthesized using procedures described previously

| | | |
|---|---|---|
| Example 138 1-[5-[5-[(1R)-1-(3,5- dichloro-4- pyridyl)ethoxy]-1H- indazol-3-yl]-3-fluoro- 2-pyridyl]-N-isobutyl- 3-methyl-azetidin-3- amine | 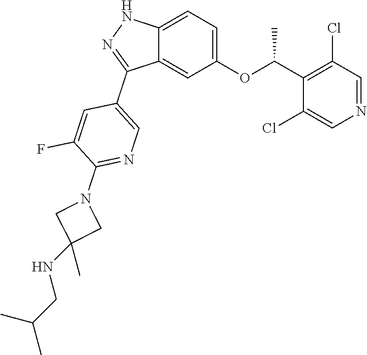 [SMILES: CC(C)CNC1(C)CN(c2ncc(-c3n[nH]c4ccc(O[C@H](C)c5c(Cl)cncc5Cl)cc34)cc2F)C1] | LCMS m/z = 543.3 (M + H); 1H NMR (400 MHz, DMSO-d6) δ 13.11 (s, 1H), 8.57 (s, 2H), 8.37 (d, J = 1.6 Hz, 1H), 7.69 (d, J = 13.2 Hz, 1H), 7.47 (d, J = 8.8 Hz, 1H), 7.20 (d, J = 1.6 Hz, 1H), 7.10 (dd, J = 8.8 Hz, 2.4 Hz, 1H), 6.14 (q, J = 6.8 Hz, 1H), 4.00 (d, J = 7.2 Hz, 2H), 3.87 (d, J = 7.2 Hz, 2H), 2.35-2.33 (m, 2H), 1.76 (d, J = 6.8 Hz, 3H), 1.63-1.58 (m, 1H), 1.44 (s, 3H), 0.90 (d, J = 6.8 Hz, 6H) |
| Example 139 (R)-5-(5-(1-(3,5- dichloropyridin-4- yl)ethoxy)-6-methoxy- 1H-indazol-3-yl)-2-(3- (isobutylamino)-3- methylazetidin-1- yl)nicotinonitrile | 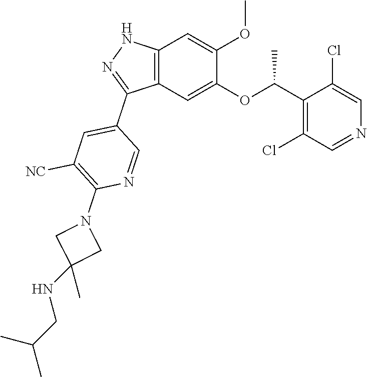 [SMILES: COc1cc2[nH]nc(-c3cnc(N4CC(C)(NCC(C)C)C4)c(C#N)c3)c2cc1O[C@H](C)c1c(Cl)cncc1Cl] | LCMS m/z = 580.5 (M + H); 1H NMR (400 MHz, DMSO-d6) δ 12.97 (s, 1H), 8.71 (d, J = 2.8 Hz, 1H), 8.58 (s, 2H), 8.03 (d, J = 2.0 Hz, 1H), 7.01 (s, 1H), 7.00 (s, 1H), 5.98 (q, J = 6.8 Hz, 1H), 4.10 (d, J = 8.4 Hz, 2H), 4.01 (d, J = 8.4 Hz, 2H), 3.86 (s, 3H), 2.32 (d, J = 8.4 Hz, 2H), 2.25 (brs, 1H), 1.75 (d, J = 6.8 Hz, 3H), 1.61-1.58 (m, 1H), 1.41 (s, 3H), 0.90 (d, J = 6.8 Hz, 6H) |
| Example 140 1-[5-[5-[(1R)-1-(3,5- dichloro-4- pyridyl)ethoxy]-1H- indazol-3-yl]-3-fluoro- 2-pyridyl]-3-methyl-N- [(1R)-1- methylpropyl]azetidin- 3-amine | 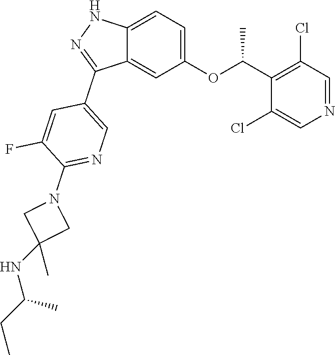 [SMILES: CC[C@@H](C)NC1(C)CN(c2ncc(-c3n[nH]c4ccc(O[C@H](C)c5c(Cl)cncc5Cl)cc34)cc2F)C1] | LCMS m/z = 543 (M + H); 1H NMR (400 MHz, DMSO-d6) δ = 9.71 (br s, 2H), 8.57 (s, 2H), 8.42 (t, J = 1.5 Hz, 1H), 7.80 (dd, J = 1.8, 13.0 Hz, 1H), 7.49 (d, J = 9.0 Hz, 1H), 7.20 (d, J = 2.2 Hz, 1H), 7.11 (dd, J = 2.3, 9.0 Hz, 1H), 6.14 (q, J = 6.7 Hz, 1H), 4.46 (dd, J = 8.7, 16.8 Hz, 2H), 4.11 (br t, J = 7.4 Hz, 2H), 3.35-3.24 (m, 1H), 1.78-1.71 (m, 7H), 1.51 (ddd, J = 7.3, 9.2, 13.5 Hz, 1H), 1.21 (d, J = 6.5 Hz, 3H), 0.95 (t, J = 7.5 Hz, 3H) |
| Example 141 1-[5-[5-[(1R)-1-(3,5- dichloro-4- pyridyl)ethoxy]-1H- indazol-3-y1]-3-fluoro- 2-pyridyl]-3-methyl-N- [(1S)-1- methylpropyl]azetidin- 3-amine | 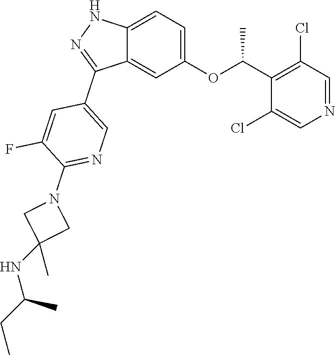 [SMILES: CC[C@H](C)NC1(C)CN(c2ncc(-c3n[nH]c4ccc(O[C@H](C)c5c(Cl)cncc5Cl)cc34)cc2F)C1] | LCMS m/z = 543.1 (M + H); 1H NMR (400 MHz, DMSO-d6) δ = 13.09 (br s, 1H), 8.57 (s, 2H), 8.38- 8.35 (m, 1H), 7.68 (dd, J = 1.7, 13.2 Hz, 1H), 7.47 (d, J = 9.0 Hz, 1H), 7.20 (d, J = 2.0 Hz, 1H), 7.10 (dd, J = 2.3, 9.0 Hz, 1H), 6.14 (q, J = 6.7 Hz, 1H), 3.96-3.87 (m, 4H), 2.72-2.66 (m, 1H), 1.76 (d, J = 6.6 Hz, 3H), 1.47 (s, 3H), 1.42- 1.32 (m, 1H), 1.29-1.17 (m, 2H), 0.96 (d, J = 6.2 Hz, 3H), 0.86 (t, J = 7.4 Hz, 3H) |
| Example 142 1-[5-[5-[(1R)-1-(3,5- dichloro-4- pyridyl)ethoxy]-1H- indazol-3-yl]-3-fluoro- 2-pyridyl]-3-methyl-N- propyl-azetidin-3- amine | 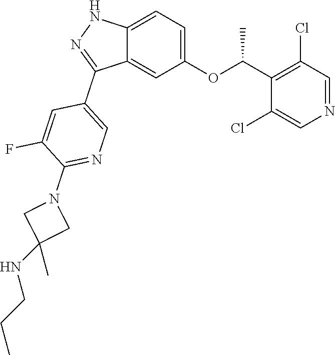 [SMILES: CCCNC1(C)CN(c2ncc(-c3n[nH]c4ccc(O[C@H](C)c5c(Cl)cncc5Cl)cc34)cc2F)C1] | LCMS m/z = 529 (M + H); 1H NMR (400 MHz, DMSO-d6) δ = 9.67 (br s, 2H), 8.57 (s, 2H), 8.41 (t, J = 1.6 Hz, 1H), 7.80 (dd, J = 1.8, 13.1 Hz, 1H), 7.49 (d, J = 9.0 Hz, 1H), 7.20 (d, J = 2.2 Hz, 1H), 7.11 (dd, J = 2.3, 9.0 Hz, 1H), 6.17- 6.11 (m, 1H), 4.44-4.40 (m, 2H), 4.09 (br d, J = 8.1 Hz, 2H), 2.97-2.88 (m, 2H), 1.76 (d, J = 6.6 Hz, 3H), 1.72-1.65 (m, 5H), 0.97 (t, J = 7.5 Hz, 3H) |
| Example 143 2-(3-amino-3-methyl- azetidin-1-yl)-5-[5- [(1R)-1-(3,5-dichloro- 4-pyridyl)ethoxy]-6- methoxy-1H-indazol-3- yl]pyridine-3- carbonitrile | 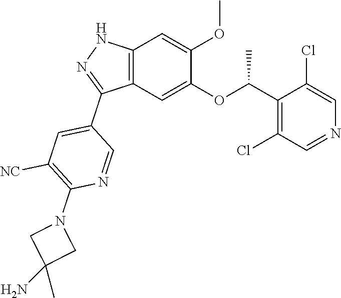 [SMILES: COc1cc2[nH]nc(-c3cnc(N4CC(C)(N)C4)c(C#N)c3)c2cc1O[C@H](C)c1c(Cl)cncc1Cl] | LCMS m/z = 524.1 (M + H); 1H NMR (400 MHz, DMSO-d6) δ = 12.97 (br s, 1H), 8.71 (s, 1H), 8.59 (s, 2H), 8.06-8.03 (m, 1H), 7.01 (d, J = 8.7 Hz, 2H), 5.99 (q, J = 6.6 Hz, 1H), 4.09 (d, J = 8.3 Hz, 2H), 4.02 (d, J = 8.6 Hz, 2H), 3.86 (s, 3H), 2.24 (br s, 2H), 1.75 (d, J = 6.5 Hz, 3H), 1.41 (s, 3H) |
| Example 144 5-[5-[(1R)-1-(3,5- dichloro-4- pyridyl)ethoxy]-6- methoxy-1H-indazol-3- yl]-2-[3-methyl-3- [[(1S)-1- methylpropyl]amino]aze- tidin-1-yl]pyridine-3- carbonitrile | 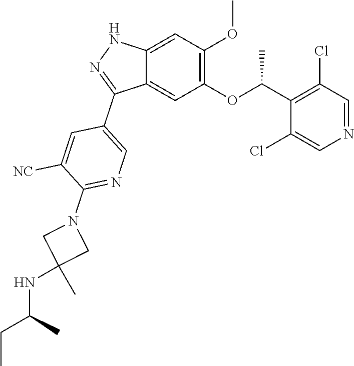 [SMILES: CC[C@H](C)NC1(C)CN(c2ncc(-c3n[nH]c4cc(OC)c(O[C@H](C)c5c(Cl)cncc5Cl)cc34)cc2C#N)C1] | LCMS m/z = 580.2 (M + H); 1H NMR (400 MHz, DMSO-d6) δ = 13.05 (br s, 1H), 9.75-9.66 (m, 1H), 9.66-9.56 (m, 1H), 8.78 (d, J = 2.3 Hz, 1H), 8.59 (s, 2H), 8.18 (d, J = 2.3 Hz, 1H), 7.06 (s, 1H), 7.02 (s, 1H), 6.03-5.97 (m, 1H), 4.58 (dd, J = 9.5, 13.9 Hz, 2H), 4.30 (dd, J = 3.2, 9.3 Hz, 2H), 3.86 (s, 3H), 3.35-3.28 (m, 1H), 1.78- 1.74 (m, 6H), 1.59-1.47 (m, 1H), 1.22 (d, J = 6.5 Hz, 3H), 0.96 (t, J = 7.5 Hz, 3H) |
| Example 145 5-[5-[(1R)-1-(3,5- dichloro-4- pyridyl)ethoxy]-6- methoxy-1H-indazol-3- yl]-2-[3-methyl-3- [[(1R)-1- methylpropyl]amino]aze- tidin-1-yl]pyridine-3- carbonitrile | 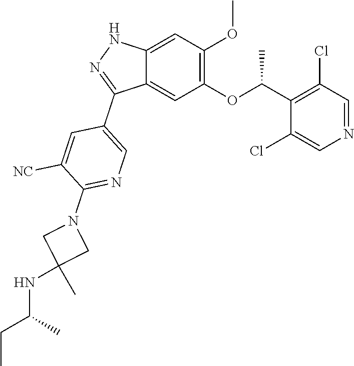 [SMILES: CC[C@@H](C)NC1(C)CN(c2ncc(-c3n[nH]c4cc(OC)c(O[C@H](C)c5c(Cl)cncc5Cl)cc34)cc2C#N)C1] | LCMS m/z = 580 (M+H); 1H NMR (400 MHz, DMSO-d6) δ = 13.05 (s, 1H), 9.75-9.66 (m, 1H), 9.66-9.58 (m, 1H), 8.78 (d, J = 2.3 Hz, 1H), 8.59 (s, 2H), 8.18 (d, J = 2.2 Hz, 1H), 7.06 (s, 1H), 7.02 (s, 1H), 6.04-5.96 (m, 1H), 4.58 (dd, J = 9.5, 14.9 Hz, 2H), 4.34- 4.26 (m, 2H), 3.86 (s, 3H), 1.78- 1.74 (m, 6H), 1.60-1.48 (m, 1H), 1.22 (d, J = 6.5 Hz, 3H), 0.96 (t, J = 7.5 Hz, 3H) |
| Example 146 5-[5-[(1R)-1-(3,5- dichloro-4- pyridyl)ethoxy]-6- methoxy-1H-indazol-3- yl]-2-[3-methyl-3- (propylamino)azetidin- 1-yl]pyridine-3- carbonitrile | 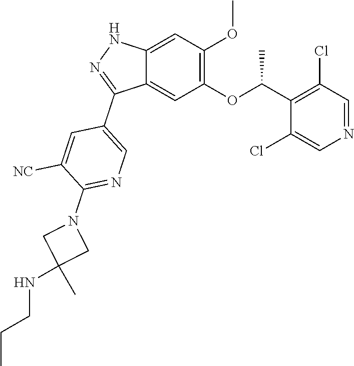 [SMILES: CCCNC1(C)CN(c2ncc(-c3n[nH]c4cc(OC)c(O[C@H](C)c5c(Cl)cncc5Cl)cc34)cc2C#N)C1] | LCMS m/z = 566 (M + H); 1H NMR (400 MHz, DMSO-d6) δ = 13.03 (s, 1H), 9.51 (br s, 2H), 8.77 (d, J = 2.2 Hz, 1H), 8.59 (s, 2H), 8.17 (d, J = 2.3 Hz, 1H), 7.05 (s, 1H), 7.02 (s, 1H), 6.00 (q, J = 6.7 Hz, 1H), 4.54 (d, J = 9.8 Hz, 2H), 4.26 (d, J = 9.8 Hz, 2H), 3.86 (s, 3H), 2.96 (br s, 2H), 1.76 (d, J = 6.7 Hz, 3H), 1.69 (s, 3H), 0.98 (t, J = 7.5 Hz, 3H) |
| Example 147 (R)-2-(3-(allylamino)- 3-methylazetidin-1-yl)- 5-(5-(1-(3,5- dichloropyridin-4- yl)ethoxy)-6-methoxy- 1H-indazol-3- yl)nicotinonitrile | 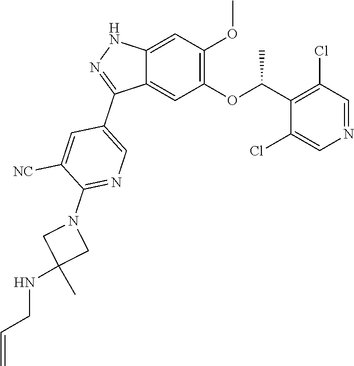 [SMILES: C=CCNC1(C)CN(c2ncc(-c3n[nH]c4cc(OC)c(O[C@H](C)c5c(Cl)cncc5Cl)cc34)cc2C#N)C1] | LCMS m/z = 564.5 (M + H); 1H NMR (400 MHz, DMSO-d6) δ 12.95 (s, 1H), 8.72 (d, J = 2.0 Hz, 1H), 8.59 (s, 2H), 8.06 (d, J = 2.8 Hz, 1H), 7.04 (s, 1H), 7.00 (s, 1H), 6.02 (q, J = 6.4 Hz, 1H), 5.94-5.87 (m, 1H), 5.25 (dd, J = 17.2 Hz, 2.0 Hz, 1H), 5.07 (dd, J = 10.0 Hz, 1.2 Hz, 1H), 4.16 (d, J = 8.8 Hz, 2H), 4.05 (d, J = 8.8 Hz, 2H), 3.86 (s, 3H), 3.22 (d, J = 6.0 Hz, 2H), 1.77 (d, J = 6.8 Hz, 3H), 1.45 (s, 3H) |
| Example 148 (R)-N-ally1-1-(5-(5-(1- (3,5-dichloropyridin-4- yl)ethoxy)-1H-indazol- 3-yl)-3-fluoropyridin- 2-yl)-3-methylazetidin- 3-amine | 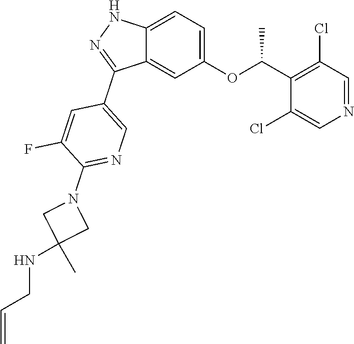 [SMILES: C=CCNC1(C)CN(c2ncc(-c3n[nH]c4ccc(O[C@H](C)c5c(Cl)cncc5Cl)cc34)cc2F)C1] | LCMS m/z = 527.2 (M + H); 1H- NMR (400 MHz, DMSO-d6) δ 13.10 (s, 1H), 8.61 (s, 2H), 8.37 (s, 1H), 7.67 (d, J = 1.6 Hz, 1H), 7.47 (d, J = 8.8 Hz, 1H), 7.20 (s, 1H), 7.10 (dd, J = 8.8 Hz, 2 Hz, 1H), 6.17 (q, J = 6.8 Hz 1H), 5.96-5.86 (m, 1H), 5.21 (d, J = 16 Hz, 1H), 5.04 (d, J = 10.4 Hz, 1H), 4.04 (d, J = 14.8 Hz, 2H), 3.86 (d, J = 8.0 Hz, 2H), 3.20 (d, J = 6.0 Hz, 2H), 1.76 (d, J = 6.4 Hz, 3H), 1.45 (s, 3H) |
| Example 149 1-[5-[5-[(1R)-1-(3,5- dichloro-4- pyridyl)ethoxy]-1H- indazol-3-yl]-3-fluoro- 2-pyridyl]-N-ethyl-3- methyl-azetidin-3- amine | 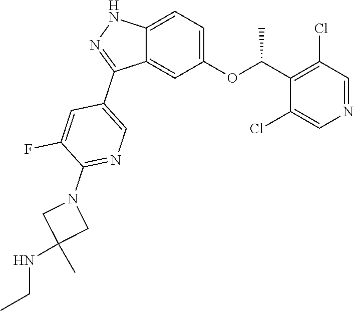 [SMILES: CCNC1(C)CN(c2ncc(-c3n[nH]c4ccc(O[C@H](C)c5c(Cl)cncc5Cl)cc34)cc2F)C1] | LCMS m/z = 515.1 (M+H); 1H NMR (400 MHz, DMSO-d6) δ = 13.22 (br s, 1H), 9.67 (br s, 2H), 8.58 (s, 2H), 8.41 (s, 1H), 7.80 (br d, J = 13.2 Hz, 1H), 7.49 (d, J = 8.7 Hz, 1H), 7.20 (s, 1H), 7.11 (br d, J = 8.9 Hz, 1H), 6.15 (q, J = 6.6 Hz, 1H), 4.40 (br d, J = 9.3 Hz, 2H), 4.09 (br d, J = 9.2 Hz, 2H), 3.02 (br s, 2H), 1.76 (br d, J = 6.5 Hz, 3H), 1.69 (s, 3H), 1.26 (br t, J = 6.7 Hz, 3H) |
| Example 150 (R)-2-(1-(5-(5-(1-(3,5- dichloropyridin-4- yl)ethoxy)-1H-indazol- 3-yl)-3-fluoropyridin- 2-yl)-3- (isopropylamino)azetidin- 3-yl)acetonitrile | 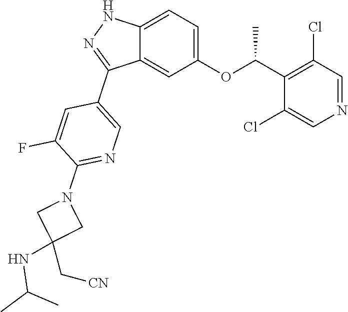 [SMILES: CC(C)NC1(CC#N)CN(c2ncc(-c3n[nH]c4ccc(O[C@H](C)c5c(Cl)cncc5Cl)cc34)cc2F)C1] | LCMS m/z = 554.3 (M + H); 1H NMR (400 MHz, DMSO-d6) δ 13.14 (s, 1H), 8.57 (s, 2H), 8.40 (s, 1H), 7.73 (dd, J = 13.2 Hz, 1.6 Hz, 1H), 7.48 (d, J = 9.2 Hz, 1H), 7.20 (s, 1H), 7.10 (dd, J = 8.8 Hz, 2.0 Hz, 1H), 6.15 (q, J = 6.4 Hz, 1H), 4.04-3.99 (m, 4H), 3.17 (s, 2H), 2.97-2.90 (m, 1H), 1.75 (d, J = 6.4 Hz, 3H), 1.01 (d, J = 6.8 Hz, 6H) |
| Example 151 2-[3- (cyclopropylmethyl- amino)-1-[5-[5-[(1R)- 1-(3,5-dichloro-4- pyridyl)ethoxy]-1H- indazol-3-yl]-3-fluoro- 2-pyridyl]azetidin-3- yl]acetonitrile | 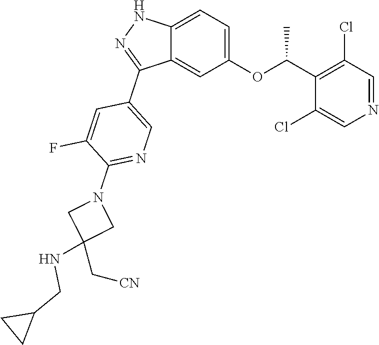 [SMILES: C[C@@H](Oc1ccc2[nH]nc(-c3cnc(N4CC(CC#N)(NCC5CC5)C4)c(F)c3)c2c1)c1c(Cl)cncc1Cl] | LCMS m/z = 566 (M + H); 1H NMR (400 MHz, DMSO-d6) δ = 13.12 (br s, 1H), 10.28 (br s, 2H), 8.57 (s, 2H), 8.44 (t, J = 1.5 Hz, 1H), 7.83 (dd, J = 1.7, 13.0 Hz, 1H), 7.49 (d, J = 9.0 Hz, 1H), 7.21 (d, J = 2.2 Hz, 1H), 7.11 (dd, J = 2.3, 9.0 Hz, 1H), 6.15 (q, J = 6.6 Hz, 1H), 4.57 (d, J = 9.7 Hz, 2H), 4.27 (d, J = 9.0 Hz, 2H), 3.58- 3.56 (m, 2H), 2.97 (br s, 2H), 1.76 (d, J = 6.6 Hz, 3H), 1.18-1.08 (m, 1H), 0.65-0.59 (m, 2H), 0.46- 0.41 (m, 2H) |
| Example 152 (R)-2-(3- (cyanomethyl)-3- (isopropylamino)azetidin- 1-yl)-5-(5-(1-(3,5- dichloropyridin-4- yl)ethoxy)-6-methoxy- 1H-indazol-3- yl)nicotinonitrile | 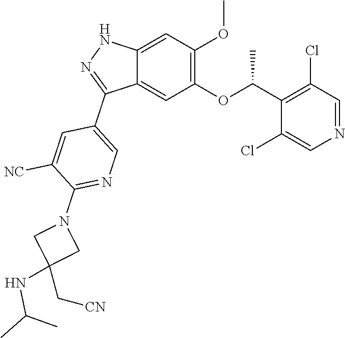 [SMILES: COc1cc2[nH]nc(-c3cnc(N4CC(CC#N)(NC(C)C)C4)c(C#N)c3)c2cc1O[C@H](C)c1c(Cl)cncc1Cl] | LCMS m/z = 591.1 (M + H); 1H NMR (400 MHz, DMSO-d6) δ 12.98 (s, 1H), 8.74 (d, J = 2.4 Hz, 1H), 8.59 (s, 2H), 8.10 (d, J = 2.0 Hz, 1H), 7.05 (s, 1H), 7.01 (s, 1H), 6.00 (q, J = 6.8 Hz, 1H), 4.22 (d, J = 8.8 Hz, 2H), 4.13 (d, J = 8.8 Hz, 2H), 3.86 (s, 3H), 3.19 (s, 2H), 2.99-2.90 (m, 1H), 1.76 (d, J = 6.8 Hz, 3H), 1.02 (d, J = 6.4 Hz, 6H) |
| Example 153 (R)-2-(3- (cyanomethyl)-3- (methylamino)azetidin- 1-yl)-5-(5-(1-(3,5- dichloropyridin-4- yl)ethoxy)-6-methoxy- 1H-indazol-3- yl)nicotinonitrile | 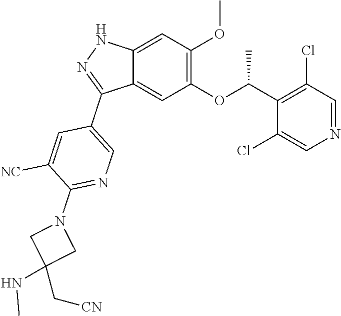 [SMILES: CNC1(CC#N)CN(c2ncc(-c3n[nH]c4cc(OC)c(O[C@H](C)c5c(Cl)cncc5Cl)cc34)cc2C#N)C1] | LCMS m/z = 563.3 (M+H); 1H NMR (400 MHz, DMSO-d6) δ 12.98 (s, 1H), 8.74 (d, J = 2.0 Hz, 1H), 8.59 (s, 2H), 8.11 (d, J= 2.4 Hz, 1H), 7.04 (s, 1H), 7.01 (s, 1H), 6.00 (q, J = 6.8 Hz, 1H), 4.17 (s, 4H), 3.86 (s, 3H), 3.14 (s, 2H), 2.28 (s, 3H), 1.76 (d, J = 6.8 Hz, 3H) |

| | | | |
|---|---|---|---|
| Example 154. | 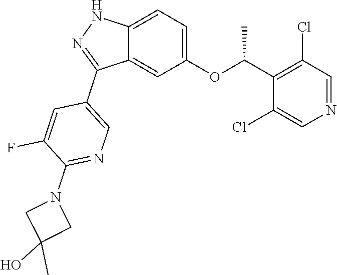 [SMILES: C[C@@H](Oc1ccc2[nH]nc(-c3cnc(N4CC(C)(O)C4)c(F)c3)c2c1)c1c(Cl)cncc1Cl] | 1-[5-[5-[(1R)-1-(3,5- dichloro-4- pyridyl)ethoxy]-1H- indazol-3-yl]-3- fluoro-2-pyridyl]-3- methyl-azetidin-3-ol | LCMS m/z = 488 (M + H); 1HNMR (400 MHz, DMSO-d6) δ 13.12 (s, 1H), 8.58 (s, 2H), 8.38 (t, J = 1.6 Hz, 1H), 7.71 (dd, J = 1.8, 13.1 Hz, 1H), 7.48 (d, J = 9.0 Hz, 1H), 7.21 (d, J = 2.0 Hz, 1H), 7.11 (dd, J = 2.3, 9.0 Hz, 1H), 6.15 (q, J = 6.6 Hz, 1H), 5.61 (s, 1H), 4.01 (d, J = 1.7 Hz, 4H), 1.77 (d, J = 6.6 Hz, 3H), 1.49 (s, 3H) |
| Example 155. | 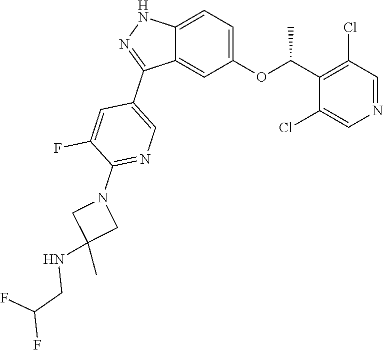 [SMILES: C[C@@H](Oc1ccc2[nH]nc(-c3cnc(N4CC(C)(NCC(F)F)C4)c(F)c3)c2c1)c1c(Cl)cncc1Cl] | 1-[5-[5-[(1R)-1-(3,5- dichloro-4- pyridyl)ethoxy]-1H- indazol-3-yl]-3- fluoro-2-pyridyl]-N- (2,2-difluoroethyl)- 3-methyl-azetidin-3- amine | LCMS m/z = 551 (M+H); 1HNMR (400 MHz, DMSO-d6) δ 13.12 (s, 1H), 8.58 (s, 2H), 8.38 (t, J = 1.5 Hz, 1H), 7.74- 7.67 (m, 1H), 7.48 (d, J = 9.0 Hz, 1H), 7.21 (d, J = 2.1 Hz, 1H), 7.11 (dd, J = 2.3, 9.0 Hz, 1H), 6.18-5.83 (m, 2H), 4.00 (d, J = 7.9 Hz, 2H), 3.89 (dd, J = 1.9, 8.4 Hz, 2H), 2.95 (ddt, J = 4.3, 7.6, 15.8 Hz, 2H), 2.78- 2.71 (m, 1H), 1.77 (d, J = 6.7 Hz, 3H), 1.45 (s, 3H) |
| Example 156. | 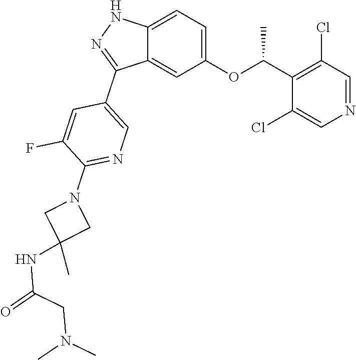 [SMILES: C[C@@H](Oc1ccc2[nH]nc(-c3cnc(N4CC(C)(NC(=O)CN(C)C)C4)c(F)c3)c2c1)c1c(Cl)cncc1Cl] | N-(1-(5-(5-(1-(3,5- dichloropyridin-4- yl)ethoxy)-1H- indazol-3-yl)-3- fluoropyridin-2-yl)- 3-methylazetidin-3- yl)-2- (dimethylamino)- acetamide | LCMS m/z = 574.2 (M + H); 1HNMR (400 MHz, DMSO-d6) δ 13.13 (s, 1H), 8.57 (s, 2H), 8.37 (t, J = 1.6 Hz, 1H), 8.35 (s, 1H), 7.71 (dd, J = 13.2 Hz, 2.0 Hz, 1H), 7.47 (d, J = 9.2 Hz, 1H), 7.20 (d, J = 1.6 Hz, 1H), 7.10 (dd, J = 8.8 Hz, 2.0 Hz, 1H), 6.14 (q, J = 6.4 Hz, 1H), 4.24 (d, J = 8.8 Hz, 2H), 4.00 (dd, J = 8.4 Hz, 1.6 Hz, 2H), 2.96 (s, 2H), 2.26 (s, 6H), 1.76 (d, J = 6.8 Hz, 3H), 1.59 (s, 3H) |
| Example 157. | 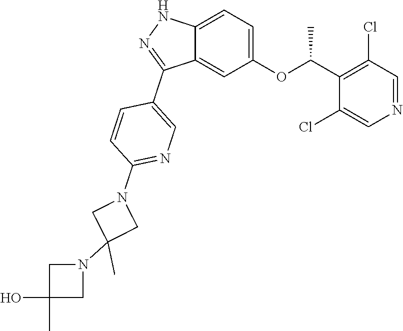 [SMILES: C[C@@H](Oc1ccc2[nH]nc(-c3ccc(N4CC(C)(N5CC(C)(O)C5)C4)nc3)c2c1)c1c(Cl)cncc1Cl] | 1-[1-[5-[5-[(1R)-1- (3,5-dichloro-4- pyridyl)ethoxy]-1H- indazol-3-yl]-2- pyridyl]-3-methyl- azetidin-3-yl]-3- methyl-azetidin-3-ol | LCMS m/z = 539.20 (M + H); 1HNMR (400 MHz, DMSO-d6) δ 8.59 (s, 2H), 8.52-8.50 (m, 1H), 7.85 (dd, J = 2.3, 8.6 Hz, 1H), 7.46 (d, J = 8.9 Hz, 1H), 7.17 (d, J = 2.1 Hz, 1H), 7.09 (dd, J = 2.3, 8.9 Hz, 1H), 6.54 (d, J = 8.6 Hz, 1H), 6.11 (q, J = 6.7 Hz, 1H), 5.31-5.10 (m, 1H), 4.06 (s, 1H), 4.04 (s, 1H), 3.67-3.61 (m, 2H), 3.20-2.95 (m, 5H), 1.76 (d, J = 6.6 Hz, 3H), 1.38 (s, 3H), 1.29 (s, 3H) |
| Example 158. | 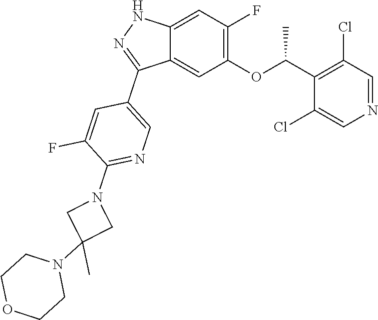 [SMILES: C[C@@H](Oc1cc2c(-c3cnc(N4CC(C)(N5CCOCC5)C4)c(F)c3)n[nH]c2cc1F)c1c(Cl)cncc1Cl] | 4-[1-[5-[5-[(1R)-1- (3,5-dichloro-4- pyridyl)ethoxy]-6- fluoro-1H-inda- zol-3-yl]-3- fluoro-2- pyridyl]-3-methyl- azetidin-3- yl]morpholine | LCMS m/z = 575.1 (M + H); 1H NMR (400 MHz, DMSO-d6) δ = 13.19 (br s, 1H), 8.60 (s, 2H), 8.34 (t, J = 1.6 Hz, 1H), 7.69 (dd, J = 1.8, 13.1 Hz, 1H), 7.45 (d, J = 10.9 Hz, 1H), 7.27 (d, J = 7.9 Hz, 1H), 6.17 (q, J = 6.6 Hz, 1H), 3.98 (d, J = 7.8 Hz, 2H), 3.81 (dd, J = 2.0, 8.2 Hz, 2H), 3.66-3.54 (m, 4H), 2.47- 2.36 (m, 4H), 1.80 (d, J = 6.7 Hz, 3H), 1.35 (s, 3H) |
| Example 159. | 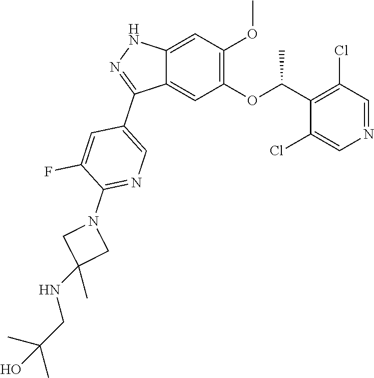 [SMILES: COc1cc2[nH]nc(-c3cnc(N4CC(C)(NCC(C)(C)O)C4)c(F)c3)c2cc1O[C@H](C)c1c(Cl)cncc1Cl] | 1-[1-[5-[5-[(1R)-1- (3,5-dichloro-4- pyridyl)ethoxy]-6- methoxy-1H- indazol-3-yl]-3- fluoro-2-pyridyl]-3- methyl-azetidin-3- yl]amino]-2-methyl- propan-2-ol | LCMS m/z = 589.3 (M + H); 1HNMR (400 MHz, DMSO-d6) δ 12.91 (s, 1H), 8.59 (s, 2H), 8.30 (s, 1H), 7.63 (d, J = 13.2 Hz, 1H), 7.08 (s, 1H), 7.00 (s, 1H), 5.99 (q, J = 6.4 Hz, 1H), 4.22 (brs, 1H), 3.99-3.97 (m, 2H), 3.88-3.87 (m, 2H), 3.86 (s, 3H), 2.43 (s, 2H), 1.76 (d, J = 6.4 Hz, 3H), 1.43 (s, 3H), 1.11 (s, 6H) |
| Example 160. | 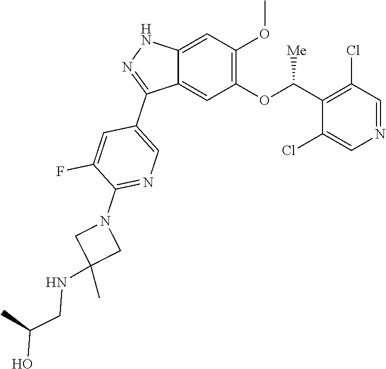 [SMILES: COc1cc2[nH]nc(-c3cnc(N4CC(C)(NC[C@H](C)O)C4)c(F)c3)c2cc1O[C@H](C)c1c(Cl)cncc1Cl] | (S)-1-((1-(5-(5-((R)- 1-(3,5- dichloropyridin-4- yl)ethoxy)-6- methoxy-1H- indazol-3-yl)-3- fluoropyridin-2-yl)- 3-methylazetidin-3- yl)amino)propan-2- ol | LCMS m/z = 575.3 (M + H); 1HNMR (400 MHz, DMSO-d6) δ 12.91 (s, 1H), 8.59 (s, 2H), 8.31 (s, 1H), 7.63 (dd, J = 13.2 Hz, 1.6 Hz, 1H), 7.08 (s, 1H), 7.00 (s, 1H), 5.99 (q, J = 6.8 Hz, 1H), 4.51 (d, J = 4.0 Hz, 1H), 3.99-3.96 (m, 2H), 3.90- 3.87 (m, 2H), 3.86 (s, 3H), 3.67-3.60 (m, 1H), 2.48-2.41 (m, 2H), 1.75 (d, J = 6.4 Hz, 3H), 1.43 (s, 3H), 1.08 (d, J = 6.4 Hz, 3H) |
| Example 161. | 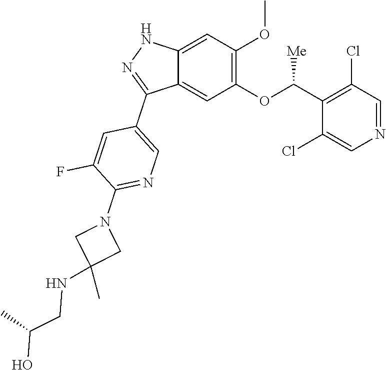 [SMILES: COc1cc2[nH]nc(-c3cnc(N4CC(C)(NC[C@@H](C)O)C4)c(F)c3)c2cc1O[C@H](C)c1c(Cl)cncc1Cl] | (R)-1-((1-(5-(5-((R)- 1-(3,5- dichloropyridin-4- yl)ethoxy)-6- methoxy-1H- indazol-3-yl)-3- fluoropyridin-2-yl)- 3-methylazetidin-3- yl)amino)propan-2- ol | LCMS m/z = 575.3 (M + H); 1HNMR (400 MHz, DMSO-d6) δ 12.92 (s, 1H), 8.59 (s, 2H), 8.30 (t, J = 1.6 Hz, 1H), 7.63 (dd, J = 13.2 Hz, 1.6 Hz, 1H), 7.08 (s, 1H), 7.00 (s, 1H), 5.99 (q, J = 6.8 Hz, 1H), 4.52 (d, J = 4.4 Hz, 1H), 4.00-3.97 (m, 2H), 3.88 (d, J = 1.6 Hz, 2H), 3.86 (s, 3H), 3.68-3.61 (m, 1H), 2.48-2.42 (m, 2H), 1.76 (d, J = 6.8 Hz, 3H), 1.43 (s, 3H), 1.08 (d, J = 6.4 Hz, 3H). |
| Example 162. | 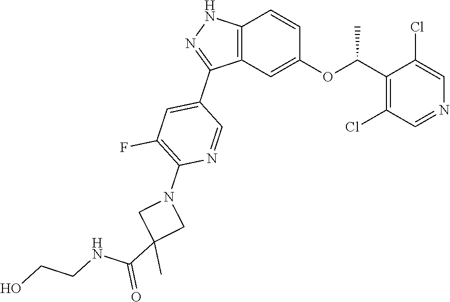 [SMILES: C[C@@H](Oc1ccc2[nH]nc(-c3cnc(N4CC(C)(C(=O)NCCO)C4)c(F)c3)c2c1)c1c(Cl)cncc1Cl] | 1-[5-[5-[(1R)-1-(3,5- dichloro-4- pyridyl)ethoxy]-1H- indazol-3-yl]-3- fluoro-2-pyridyl]-N- (2-hydroxyethyl)-3- methyl-azetidine-3- carboxamide | LCMS m/z = 559.4 (M + H); 1HNMR (400 MHz, DMSO-d6) δ 13.12 (s, 1H), 8.57 (s, 2H), 8.37 (t, J = 1.6 Hz, 1H), 7.93 (t, J = 5.6 Hz, 1H), 7.71 (dd, J = 12.8 Hz, 1.6 Hz, 1H), 7.47 (d, J = 8.8 Hz, 1H), 7.20 (d, J = 1.6 Hz, 1H), 7.10 (dd, J = 9.2 Hz, 2.0 Hz, 1H), 6.14 (q, J = 6.8 Hz, 1H), 4.68 (brs, 1H), 4.34 (d, J = 7.6 Hz, 2H), 3.88 (dd, J = 8.4 Hz, 1.6 Hz, 2H), 3.43 (t, J = 6.0 Hz, 2H), 3.18 (q, J = 6.0 Hz, 2H), 1.76 (d, J = 6.4 Hz, 3H), 1.55 (s, 3H) |
| Example 163. | 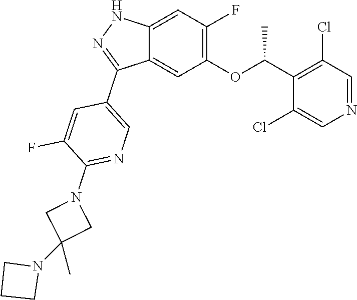 [SMILES: C[C@@H](Oc1cc2c(-c3cnc(N4CC(C)(N5CCC5)C4)c(F)c3)n[nH]c2cc1F)c1c(Cl)cncc1Cl] | 3-[6-[3-(azetidin-1- yl)-3-methyl- azetidin-1-yl]-5- fluoro-3-pyridyl]-5- [(1R)-1-(3,5- dichloro-4- pyridyl)ethoxy]-6- fluoro-1H-indazole | LCMS m/z = 545 (M + H); 1H NMR (400 MHz, DMSO-d6) δ 13.18 (br s, 1H), 8.61 (s, 2H), 8.35 (t, J = 1.6 Hz, 1H), 7.69 (dd, J = 1.8, 13.1 Hz, 1H), 7.46 (d, J = 10.9 Hz, 1H), 7.29 (d, J = 7.8 Hz, 1H), 6.17 (q, J = 6.6 Hz, 1H), 4.21 (br d, J = 8.7 Hz, 2H), 3.79 (br d, J = 8.7 Hz, 2H), 3.31-3.17 (m, 4H), 2.04-1.91 (m, 2H), 1.81 (d, J = 6.7 Hz, 3H), 1.29 (s, 3H) |
| Example 164. | 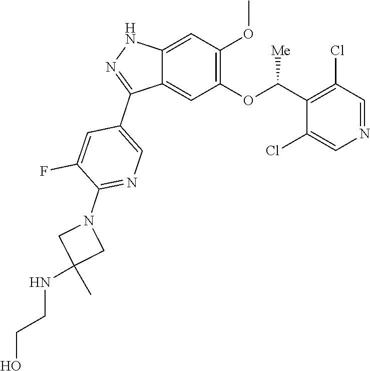 [SMILES: COc1cc2[nH]nc(-c3cnc(N4CC(C)(NCCO)C4)c(F)c3)c2cc1O[C@H](C)c1c(Cl)cncc1Cl] | (R)-2-((1-(5-(5-(1- (3,5-dichloro- pyridin- 4-yl)ethoxy)-6- methoxy-1H- indazol-3-yl)-3- fluoropyridin-2-yl)- 3-methylazetidin-3- yl)amino)ethan-1-ol | LCMS m/z = 561.2 (M + H); 1H NMR (400 MHz, DMSO-d6) δ 12.92 (s, 1H), 8.59 (s, 2H), 8.30 (s, 1H), 7.64 (dd, J = 13.2 Hz, 1.6 Hz, 1H), 7.08 (s, 1H), 7.00 (s, 1H), 5.98 (q, J = 6.8 Hz, 1H), 4.62 (brs, 1H), 4.01 (d, J = 7.6 Hz, 2H), 3.95-3.87 (m, 2H), 3.86 (s, 3H), 3.55-3.47 (m, 2H), 2.68-2.64 (m, 2H), 1.75 (d, J = 6.8 Hz, 3H), 1.45 (s, 3H). |
| Example 165. | 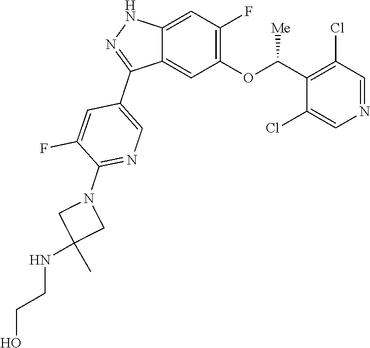 [SMILES: C[C@@H](Oc1cc2c(-c3cnc(N4CC(C)(NCCO)C4)c(F)c3)n[nH]c2cc1F)c1c(Cl)cncc1Cl] | 2-[1-[5-[5-[(1R)-1- (3,5-dichloro-4- pyridyl)ethoxy]-6- fluoro-1H-indazol-3-yl]- 3-fluoro-2- pyridyl]-3-methyl- azetidin-3- yl]amino]ethanol | LCMS m/z = 549 (M + H); 1H NMR (400 MHz, DMSO-d6) δ 9.22 (br s, 2H), 8.60 (s, 2H), 8.40 (t, J = 1.5 Hz, 1H), 7.78 (dd, J = 1.8, 13.0 Hz, 1H), 7.48 (d, J = 10.8 Hz, 1H), 7.27 (d, J = 7.8 Hz, 1H), 6.17 (q, J = 6.7 Hz, 1H), 4.37 (br d, J = 9.3 Hz, 2H), 4.10 (d, J = 9.4 Hz, 2H), 3.70 (t, J = 5.0 Hz, 2H), 3.10 (br s, 2H), 1.81 (d, J = 6.6 Hz, 3H), 1.68 (s, 3H) |
| Example 166. | 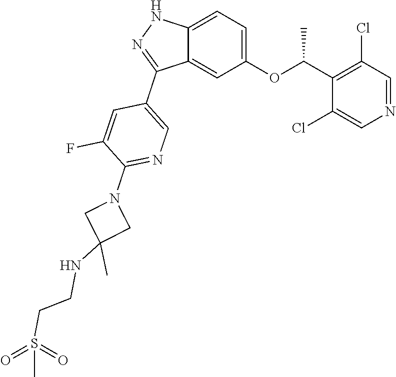 [SMILES: C[C@@H](Oc1ccc2[nH]nc(-c3cnc(N4CC(C)(NCCS(C)(=O)=O)C4)c(F)c3)c2c1)c1c(Cl)cncc1Cl] | 1-[5-[5-[(1R)-1-(3,5- dichloro-4- pyridyl)ethoxy]-1H- indazol-3-yl]-3- fluoro-2-pyridyl]-3- methyl-N-(2- methylsulfonylethyl) azetidin-3-amine | LCMS m/z = 593 (M + H); 1HNMR (400 MHz, DMSO-d6) δ 13.03 (s, 1H), 8.50 (s, 2H), 8.30 (t, J = 1.5 Hz, 1H), 7.63 (dd, J = 1.7, 13.2 Hz, 1H), 7.40 (d, J = 9.0 Hz, 1H), 7.13 (d, J = 2.0 Hz, 1H), 7.03 (dd, J = 2.3, 9.0 Hz, 1H), 6.07 (q, J = 6.7 Hz, 1H), 3.93 (d, J = 7.8 Hz, 2H), 3.83 (dd, J = 2.0, 8.3 Hz, 2H), 3.15 (t, J = 6.7 Hz, 2H), 2.97 (s, 3H), 2.92-2.87 (m, 2H), 2.51 (br s, 1H), 1.69 (d, J = 6.6 Hz, 3H), 1.38 (s, 3H) |
| Example 167. | 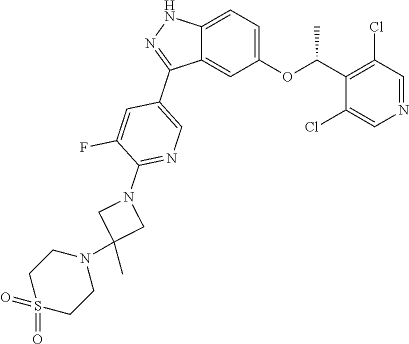 [SMILES: C[C@@H](Oc1ccc2[nH]nc(-c3cnc(N4CC(C)(N5CCS(=O)(=O)CC5)C4)c(F)c3)c2c1)c1c(Cl)cncc1Cl] | 4-[1-[5-[5-[(1R)-1- (3,5-dichloro-4- pyridyl)ethoxy]-1H- indazol-3-yl]-3- fluoro-2-pyridyl]-3- methyl-azetidin-3- yl]-1,4-thiazinane 1,1-dioxide | LCMS m/z = 605 (M + H); 1HNMR (400 MHz, DMSO-d6) δ 13.05 (br s, 1H), 8.50 (s, 2H), 8.30 (t, J = 1.5 Hz, 1H), 7.66 (dd, J = 1.8, 13.1 Hz, 1H), 7.41 (d, J = 9.0 Hz, 1H), 7.13 (d, J = 1.8 Hz, 1H), 7.04 (dd, J = 2.3, 9.0 Hz, 1H), 6.07 (q, J = 6.6 Hz, 1H), 3.93 (d, J = 7.9 Hz, 2H), 3.79 (dd, J = 1.9, 8.1 Hz, 2H), 3.08-3.02 (m, 4H), 2.76 (br s, 4H), 1.69 (d, J = 6.7 Hz, 3H), 1.32 (s, 3H) |
| Example 168. | 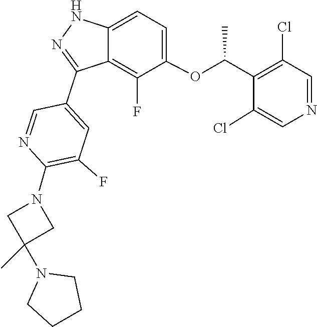 [SMILES: C[C@@H](Oc1ccc2[nH]nc(-c3cnc(N4CC(C)(N5CCCC5)C4)c(F)c3)c2c1F)c1c(Cl)cncc1Cl] | (R)-5-(1-(3,5- dichloropyridin-4- yl)ethoxy)-4-fluoro- 3-(5-fluoro-6-(3- methyl-3- (pyrrolidin-1- yl)azetidin-1- yl)pyridin-3-yl)-1H- indazole | LCMS m/z = 559.2 (M + H); 1HNMR (400 MHz, DMSO-d6) δ 13.44 (s, 1H), 8.62 (s, 2H), 8.32 (d, J = 1.6 Hz, 1H), 7.74 (d, J = 13.2 Hz, 1H), 7.27 (d, J = 9.2 Hz, 1H), 7.13-7.10 (m, 1H), 5.99 (q, J = 6.8 Hz, 1H), 4.27-4.12 (m, 2H), 3.92-3.75 (m, 2H), 2.76-2.57 (m, 4H), 1.79 (d, J = 6.4 Hz, 3H), 1.72- 1.67 (m, 4H), 1.44 (s, 3H) |
| Example 169. | 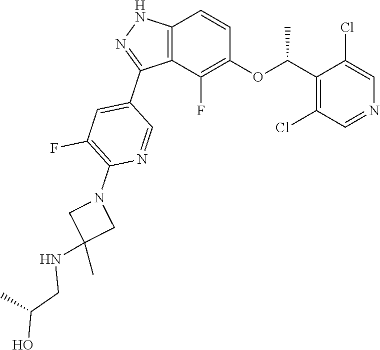 [SMILES: C[C@@H](O)CNC1(C)CN(c2ncc(-c3n[nH]c4ccc(O[C@H](C)c5c(Cl)cncc5Cl)c(F)c34)cc2F)C1] | (R)-1-((1-(5-(5-((R)- 1-(3,5- dichloropyridin-4- yl)ethoxy)-4-fluoro- 1H-indazol-3-yl)-3- fluoropyridin-2-yl)- 3-methylazetidin-3- yl)amino)propan-2- ol | LCMS m/z = 563.2 (M + H); 1HNMR (400 MHz, DMSO-d6) δ 13.44 (s, 1H), 8.61 (s, 2H), 8.32 (d, J = 1.2 Hz, 1H), 7.74 (d, J = 13.2 Hz, 1H), 7.27 (d, J = 8.8 Hz, 1H), 7.13 (t, J = 7.6 Hz, 1H), 5.99 (q, J = 6.8 Hz, 1H), 4.52 (brs, 1H), 4.00-3.97 (m, 2H), 3.87 (dd, J = 8.4 Hz, 1.6 Hz, 2H), 3.65-3.61 (m, 1H), 2.46-2.41 (m, 2H), 1.79 (d, J = 6.8 Hz, 3H), 1.43 (s, 3H), 1.07 (d, J = 6.4 Hz, 3H) |
| Example 170. | 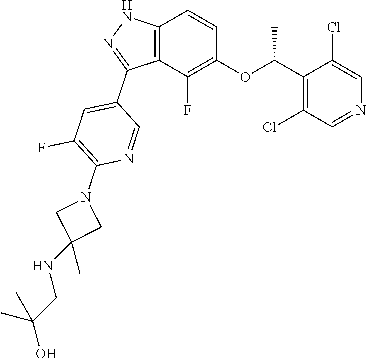 [SMILES: C[C@@H](Oc1ccc2[nH]nc(-c3cnc(N4CC(C)(NCC(C)(C)O)C4)c(F)c3)c2c1F)c1c(Cl)cncc1Cl] | (R)-1-((1-(5-(5-(1- (3,5-dichloro- pyridin- 4-yl)ethoxy)-4- fluoro-1H-indazol-3- yl)-3-fluoropyridin- 2-yl)-3- methylazetidin-3- yl)amino)-2- methylpropan-2-ol | LCMS m/z = 577.3 (M + H); 1HNMR (400 MHz, DMSO-d6) δ 13.44 (s, 1H), 8.61 (s, 2H), 8.32 (d, J = 1.2 Hz, 1H), 7.74 (d, J = 13.2 Hz, 1H), 7.27 (d, J = 9.2 Hz, 1H), 7.13 (t, J = 7.6 Hz, 1H), 5.99 (q, J = 6.4 Hz, 1H), 4.21 (brs, 1H), 4.03-3.95 (m, 2H), 3.90-3.85 (m, 2H), 2.46-2.42 (m, 2H), 1.79 (d, J = 6.8 Hz, 3H), 1.43 (s, 3H), 1.11 (s, 6H) |
| Example 171. | 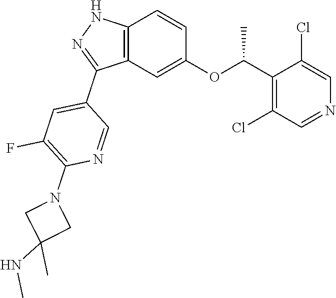 [SMILES: CNC1(C)CN(c2ncc(-c3n[nH]c4ccc(O[C@H](C)c5c(Cl)cncc5Cl)cc34)cc2F)C1] | (R)-1-(5-(5-(1-(3,5- dichloropyridin-4- yl)ethoxy)-1H- indazol-3-yl)-3- fluoropyridin-2-yl)- N,3- dimethylazetidin-3- amine | LCMS m/z = 501.1 (M + H); 1HNMR (400 MHz, DMSO-d6) δ 13.12 (s, 1H), 8.57 (s, 2H), 8.37 (s, 1H), 7.70 (dd, J = 13.2 Hz, 1.2 Hz, 1H), 7.47 (d, J = 8.8 Hz, 1H), 7.19 (s, 1H), 7.10 (dd, J = 8.4, 1.2 Hz, 1H), 6.14 (q, J = 6.0 Hz, 1H), 4.01 (d, J = 8.4 Hz, 2H), 3.88 (d, J = 8.0 Hz, 2H), 2.29 (s, 3H), 1.76 (d, J = 6.4 Hz, 3H), 1.43 (s, 3H) |
| Example 172. | 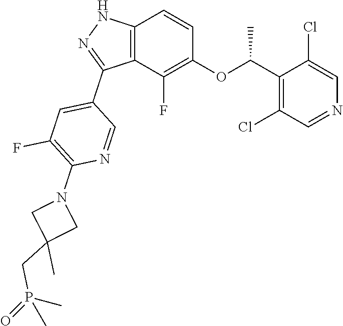 [SMILES: C[C@@H](Oc1ccc2[nH]nc(-c3cnc(N4CC(C)(CP(C)(C)=O)C4)c(F)c3)c2c1F)c1c(Cl)cncc1Cl] | (R)-((1-(5-(5-(1- (3,5-dichloro- pyridin-4- yl)ethoxy)-4- fluoro-1H-inda- zol-3- yl)-3-fluoropyridin- 2-yl)-3- methylazetidin-3- yl)methyl)dimethyl- phosphine oxide | LCMS m/z = 580.2 (M + H); 1HNMR (400 MHz, DMSO-d6) δ 13.43 (brs, 1H), 8.61 (s, 2H), 8.32 (d, J = 0.8 Hz, 1H), 7.73 (d, J = 13.2 Hz, 1H), 7.26 (d, J = 8.8 Hz, 1H), 7.14-7.10 (m, 1H), 5.99 (q, J = 6.4 Hz, 1H), 4.14-4.08 (m, 2H), 3.90-3.84 (m, 2H), 2.19 (d, J = 11.2 Hz, 2H), 1.79 (d, J = 6.8 Hz, 3H), 1.55 (s, 3H), 1.45 (s, 3H), 1.42 (s, 3H) |
| Example 173. | 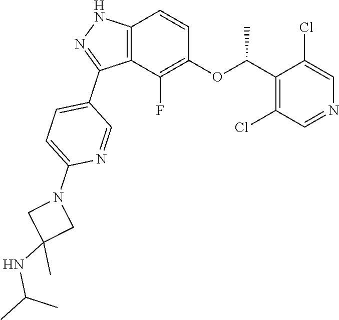 [SMILES: CC(C)NC1(C)CN(c2ccc(-c3n[nH]c4ccc(O[C@H](C)c5c(Cl)cncc5Cl)c(F)c34)cn2)C1] | (R)-1-(5-(5-(1-(3,5- dichloropyridin-4- yl)ethoxy)-4-fluoro- 1H-indazol-3- yl)pyridin-2-yl)-N- isopropyl-3- methylazetidin-3- amine | LCMS m/z = 529.2 (M + H); 1HNMR (400 MHz, DMSO-d6) δ 13.32 (s, 1H), 8.61 (s, 2H), 8.44 (s, 1H), 7.87 (d, J = 8.4 Hz, 1H), 7.25 (d, J = 8.8 Hz, 1H), 7.13 (t, J = 8.0 Hz, 1H), 6.50 (d, J = 8.4 Hz, 1H), 6.00 (q, J = 6.8 Hz, 1H), 3.80 (d, J = 7.6 Hz, 2H), 3.74 (d, J = 7.6 Hz, 2H), 2.97-2.90 (m, 1H), 1.80 (d, J = 6.4 Hz, 3H), 1.45 (s, 3H), 0.99 (d, J = 6.0 Hz, 6H) |
| Example 174. | 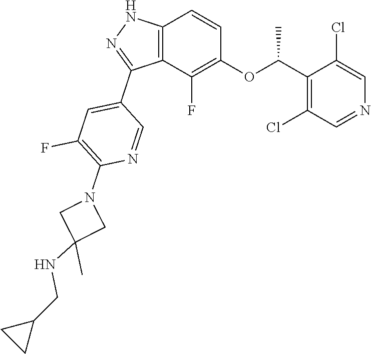 [SMILES: C[C@@H](Oc1ccc2[nH]nc(-c3cnc(N4CC(C)(NCC5CC5)C4)c(F)c3)c2c1F)c1c(Cl)cncc1Cl] | (R)-N-(cyclo- propylmethyl)- 1-(5-(5-(1-(3,5- dichloropyridin-4- yl)ethoxy)-4-fluoro- 1H-indazol-3-yl)-3- fluoropyridin-2-yl)- 3-methylazetidin-3- amine | LCMS m/z = 559.2 (M + H); 1HNMR (400 MHz, DMSO-d6) δ 13.44 (s, 1H), 8.61 (s, 2H), 8.32 (d, J = 1.6 Hz, 1H), 7.73 (dd, J = 8.7 Hz, 2.0 Hz, 1H), 7.27 (d, J = 8.8 Hz, 1H), 7.13 (dd, J = 9.2 Hz, 8.0 Hz, 1H), 5.99 (q, J = 6.8 Hz, 1H), 3.99 (d, J = 7.6 Hz, 2H), 3.84 (dd, J = 8.0 Hz, 1.6 Hz, 2H), 2.40 (d, J = 6.8 Hz, 2H), 1.80 (d, J = 6.8 Hz, 3H), 1.42 (s, 3H), 0.91-0.82 (m, 1H), 0.43-0.38 (m, 2H), 0.18-0.10 (m, 2H) |
| Example 175. | 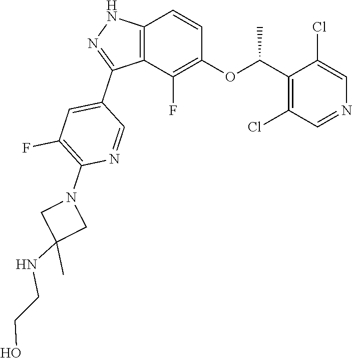 [SMILES: C[C@@H](Oc1ccc2[nH]nc(-c3cnc(N4CC(C)(NCCO)C4)c(F)c3)c2c1F)c1c(Cl)cncc1Cl] | (R)-2-((1-(5-(5-(1- (3,5-dichloro- pyridin- 4-yl)ethoxy)-4- fluoro-1H-indazol-3-yl)-3- fluoropyridin- 2-yl)-3- methylazetidin-3- yl)amino)ethan-1-ol | LCMS m/z = 549.2 (M + H); 1HNMR (400 MHz, DMSO-d6) δ 13.43 (brs, 1H), 8.61 (s, 2H), 8.32 (d, J = 1.2 Hz, 1H), 7.74 (d, J = 13.6 Hz, 1H), 7.26 (d, J = 8.8 Hz, 1H), 7.12 (dd, J = 8.8 Hz, 7.2 Hz, 1H), 5.99 (q, J = 6.8 Hz, 1H), 4.51 (t, J = 5.2 Hz, 1H), 3.96 (d, J = 8.0 Hz, 2H), 3.87 (dd, J = 8.4 Hz, 2.0 Hz, 2H), 3.46 (q, J = 5.6 Hz, 2H), 2.60 (t, J = 6.0 Hz, 2H), 2.23 (brs, 1H), 1.79 (d, J = 6.8 Hz, 3H), 1.42 (s, 3H) |
| Example 176. | 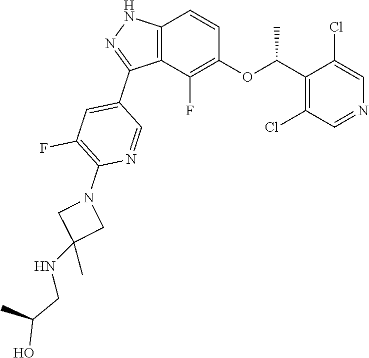 [SMILES: C[C@H](O)CNC1(C)CN(c2ncc(-c3n[nH]c4ccc(O[C@H](C)c5c(Cl)cncc5Cl)c(F)c34)cc2F)C1] | (S)-1-((1-(5-(5-((R)- 1-(3,5- dichloropyridin-4- yl)ethoxy)-4-fluoro- 1H-indazol-3-yl)-3- fluoropyridin-2-yl)- 3-methylazetidin-3- yl)amino)propan-2- ol | LCMS m/z = 563.2 (M + H); 1HNMR (400 MHz, DMSO-d6) δ 13.43 (brs, 1H), 8.61 (s, 2H), 8.32 (dd, J = 3.2 Hz, 1.6 Hz, 1H), 7.75-7.71 (m, 1H), 7.26 (d, J = 8.8 Hz, 1H), 7.15-7.10 (m, 1H), 5.99 (q, J = 6.8 Hz, 1H), 4.48 (d, J = 4.4 Hz, 1H), 3.99- 3.96 (m, 2H), 3.86 (dd, J = 7.6 Hz, 1.6 Hz, 2H), 3.66-3.57 (m, 1H), 2.47-2.40 (m, 2H), 2.17 (brs, 1H), 1.79 (d, J = 6.4 Hz, 3H), 1.41 (s, 3H), 1.07 (d, J = 6.0 Hz, 3H) |
| Example 177. | 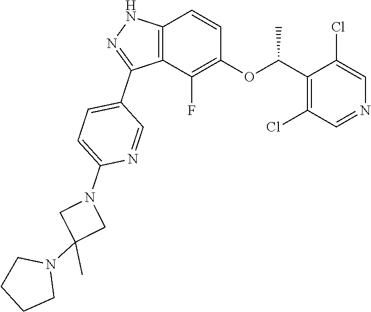 [SMILES: C[C@@H](Oc1ccc2[nH]nc(-c3ccc(N4CC(C)(N5CCCC5)C4)nc3)c2c1F)c1c(Cl)cncc1Cl] | (R)-5-(1-(3,5- dichloropyridin-4- yl)ethoxy)-4-fluoro- 3-(6-(3-methyl-3- (pyrrolidin-1- yl)azetidin-1- yl)pyridin-3-yl)-1H- indazole | LCMS m/z = 541.2 (M + H); 1HNMR (400 MHz, DMSO-d6) δ 13.31 (brs, 1H), 8.61 (s, 2H), 8.45 (t, J = 1.6 Hz, 1H), 7.87 (dt, J = 8.4 Hz, 2.0 Hz, 1H), 7.24 (d, J = 9.2 Hz, 1H), 7.11 (dd, J = 8.8 Hz, 7.2 Hz, 1H), 6.49 (dd, J = 8.8 Hz, 0.8 Hz, 1H), 5.98 (q, J = 6.8 Hz, 1H), 4.02 (d, J = 8.4 Hz, 2H), 3.67 (d, J = 8.0 Hz, 2H), 2.65-2.57 (m, 4H), 1.79 (d, J = 6.8 Hz, 3H), 1.74-1.66 (m, 4H), 1.42 (s, 3H) |
| Example 178. | 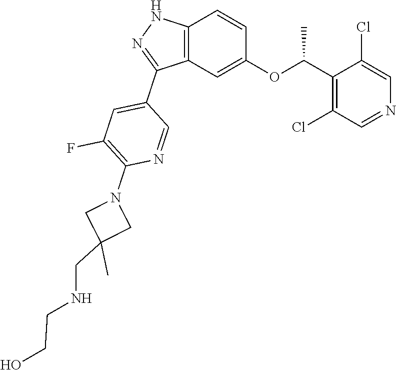 [SMILES: C[C@@H](Oc1ccc2[nH]nc(-c3cnc(N4CC(C)(CNCCO)C4)c(F)c3)c2c1)c1c(Cl)cncc1Cl] | (R)-2-(((1-(5-(5-(1- (3,5-dichloro- pyridin- 4-yl)ethoxy)-1H- indazol-3-yl)-3- fluoropyridin-2-yl)- 3-methylazetidin-3- yl)methyl)amino) ethanol | LCMS m/z = 545.2 (M + H); 1HNMR (400 MHz, DMSO-d6) δ 13.09 (brs, 1H), 8.56 (s, 2H), 8.34 (t, J = 1.6 Hz, 1H), 7.67 (dd, J = 13.2 Hz, 2.0 Hz, 1H), 7.46 (d, J = 9.2 Hz, 1H), 7.19 (d, J = 2.0 Hz, 1H), 7.09 (dd, J = 9.2 Hz, 2.4 Hz, 1H), 6.13 (q, J = 6.8 Hz, 1H), 4.46 (t, J = 5.6 Hz, 1H), 3.98-3.95 (m, 2H), 3.76-3.73 (m, 2H), 3.75 (q, J = 2.0 Hz, 1H), 2.70 (s, 2H), 2.65- 2.62 (m, 2H), 1.75 (d, J = 6.8 Hz, 3H), 1.31 (s, 3H) |
| Example 179. | 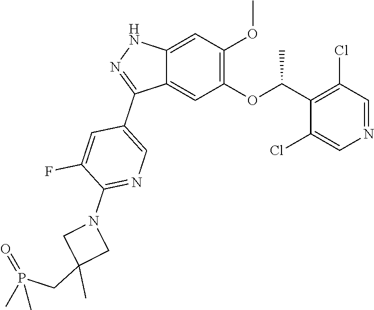 [SMILES: COc1cc2[nH]nc(-c3cnc(N4CC(C)(CP(C)(C)=O)C4)c(F)c3)c2cc1O[C@H](C)c1c(Cl)cncc1Cl] | (R)-((1-(5-(5-(1- (3,5-dichloro- pyridin- 4-yl)ethoxy)-6- methoxy-1H- indazol-3-yl)-3- fluoropyridin-2-yl)- 3-methylazetidin-3- yl)methyl)dimethyl- phosphine oxide | LCMS m/z = 592 (M + H); 1HNMR (400 MHz, DMSO-d6) δ 12.92 (brs, 1H), 8.59 (s, 2H), 8.30-8.29 (m, 1H), 7.63 (dd, J = 13.2 Hz, 2.0 Hz, 1H), 7.07 (s, 1H), 7.00 (s, 1H), 5.98 (q, J = 6.4 Hz, 1H), 4.11-4.10 (m, 2H), 3.87-3.86 (m, 5H), 2.20 (d, J = 11.2 Hz, 2H), 1.76 (d, J = 6.8 Hz, 3H), 1.56 (s, 3H), 1.45 (s, 3H), 1.42 (s, 3H) |
| Example 180. | 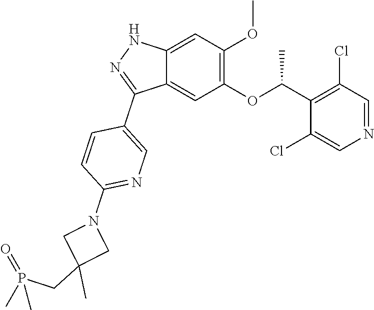 [SMILES: COc1cc2[nH]nc(-c3ccc(N4CC(C)(CP(C)(C)=O)C4)nc3)c2cc1O[C@H](C)c1c(Cl)cncc1Cl] | (R)-((1-(5-(5-(1- (3,5-dichloro- pyridin-4-yl) ethoxy)-6-meth- oxy-1H-inda- zol-3-yl)pyridin- 2-yl)-3- methylazetidin-3- yl)methyl)dimethyl- phosphine oxide | LCMS m/z = 574.3 (M + H); 1HNMR (400 MHz, DMSO-d6) δ 12.79 (s, 1H), 8.62 (s, 2H), 8.44 (d, J = 2.0 Hz, 1H), 7.79 (dd, J = 8.4 Hz, 2.0 Hz, 1H), 7.05 (s, 1H), 6.99 (s, 1H), 6.50 (d, J = 8.8 Hz, 1H), 5.99 (q, J = 6.4 Hz, 1H), 3.96 (d, J = 8.0 Hz, 2H), 3.86 (s, 3H), 3.76 (d, J = 7.6 Hz, 2H), 2.20 (d, J = 11.6 Hz, 2H), 1.76 (d, J = 6.8 Hz, 3H), 1.55 (s, 3H), 1.46 (s, 3H), 1.42 (s, 3H) |
| Example 181. | 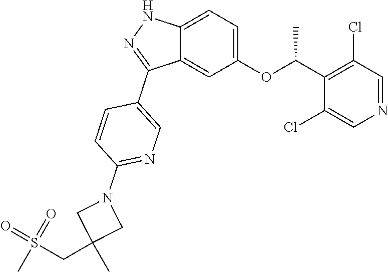 [SMILES: C[C@@H](Oc1ccc2[nH]nc(-c3ccc(N4CC(C)(CS(C)(=O)=O)C4)nc3)c2c1)c1c(Cl)cncc1Cl] | (R)-5-(1-(3,5- dichloropyridin-4- yl)ethoxy)-3-(6-(3- methyl-3- ((methylsulfonyl)- methyl)azetidin-1- yl)pyridin-3-yl)-1H- indazole | LCMS m/z = 546.2 (M + H); 1HNMR (400 MHz, DMSO-d6) δ 13.00 (s, 1H), 8.60 (s, 2H), 8.51 (d, J = 2.0 Hz, 1H), 7.86 (dd, J = 8.8 Hz, 2.0 Hz, 1H), 7.47 (d, J = 9.2 Hz, 1H), 7.16 (d, J = 2.0 Hz, 1H), 7.10 (dd, J = 8.8 Hz, 2.4 Hz, 1H), 6.56 (d, J = 8.4 Hz, 1H), 6.13 (q, J = 6.4 Hz, 1H), 4.07 (d, J = 8.0 Hz, 2H), 3.79 (d, J = 8.4 Hz, 2H), 3.67 (s, 2H), 3.03 (s, 3H), 1.77 (d, J = 6.4 Hz, 3H), 1.61 (s, 3H) |
| Example 182. | 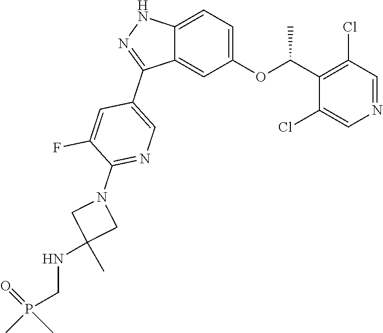 [SMILES: C[C@@H](Oc1ccc2[nH]nc(-c3cnc(N4CC(C)(NCP(C)(C)=O)C4)c(F)c3)c2c1)c1c(Cl)cncc1Cl] | 1-[5-[5-[(1R)-1-(3,5- dichloro-4- pyridyl)ethoxy]-1H- indazol-3-yl]-3- fluoro-2-pyridyl]-N- (dimethylphosphoryl methyl)-3-methyl- azetidin-3-amine | LCMS m/z = 577 (M + H); 1HNMR (400 MHz, DMSO-d6) δ 13.04 (br s, 1H), 8.50 (s, 2H), 8.30 (t, J = 1.5 Hz, 1H), 7.62 (dd, J = 1.8, 13.2 Hz, 1H), 7.40 (d, J = 9.0 Hz, 1H), 7.13 (d, J = 2.0 Hz, 1H), 7.03 (dd, J = 2.3, 9.0 Hz, 1H), 6.07 (q, J = 6.6 Hz, 1H), 3.97 (d, J = 7.9 Hz, 2H), 3.80 (dd, J = 1.8, 8.3 Hz, 2H), 2.78 (t, J = 8.8 Hz, 2H), 2.42- 2.38 (m, 1H), 1.69 (d, J = 6.6 Hz, 3H), 1.38 (s, 3H), 1.33 (d, J = 13.0 Hz, 6H) |
| Example 183. | 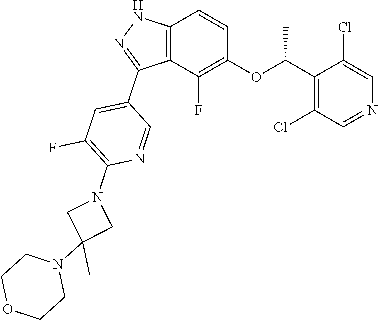 [SMILES: C[C@@H](Oc1ccc2[nH]nc(-c3cnc(N4CC(C)(N5CCOCC5)C4)c(F)c3)c2c1F)c1c(Cl)cncc1Cl] | (R)-4-(1-(5-(5-(1- (3,5-dichloro- pyridin-4-yl) ethoxy)-4-fluoro- 1H-indazol-3-yl)- 3-fluoropyridin- 2-yl)-3- methylazetidin-3- yl)morpholine | LCMS m/z = 575.2 (M + H); 1HNMR (400 MHz, DMSO-d6) δ 13.44 (s, 1H), 8.61 (s, 2H), 8.32 (d, J = 1.2 Hz, 1H), 7.75 (d, J = 13.2 Hz, 1H), 7.26 (d, J = 8.8 Hz, 1H), 7.14-7.10 (m, 1H), 5.99 (q, J = 6.8 Hz, 1H), 4.00-3.96 (m, 2H), 3.80-3.77 (m, 2H), 3.63-3.55 (m, 4H), 2.49 (t, J = 2.0 Hz, 4H), 1.79 (d, J = 6.8 Hz, 3H), 1.34 (s, 3H) |
| Example 184. | 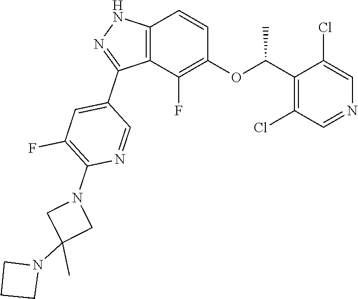 [SMILES: C[C@@H](Oc1ccc2[nH]nc(-c3cnc(N4CC(C)(N5CCC5)C4)c(F)c3)c2c1F)c1c(Cl)cncc1Cl] | (R)-5-(1-(3,5- dichloropyridin-4- yl)ethoxy)-4-fluoro- 3-(5-fluoro-6-(3′- methyl-[1,3′- biazetidin]-1′- yl)pyridin-3-yl)-1H- indazole | LCMS m/z = 545.2 (M + H); 1HNMR (400 MHz, DMSO-d6) δ 13.44 (s, 1H), 8.61 (s, 2H), 8.32 (d, J = 1.6 Hz, 1H), 7.72 (d, J = 13.2 Hz, 1H), 7.28 (d, J = 8.8 Hz, 1H), 7.14 (t, J = 8.8 Hz, 1H), 6.01 (q, J = 6.8 Hz, 1H), 4.19 (d, J = 8.8 Hz, 2H), 3.75 (dd, J = 8.8 Hz, 2.0 Hz, 2H), 3.23 (t, J = 6.8 Hz, 4H), 1.98-1.90 (m, 2H), 1.80 (d, J = 6.8 Hz, 3H), 1.26 (s, 3H) |
| Example 185. | 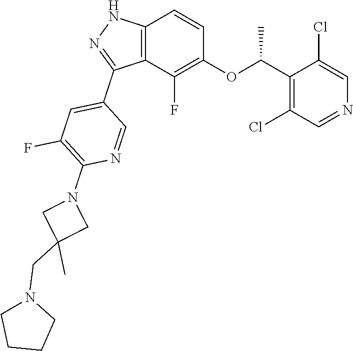 [SMILES: C[C@@H](Oc1ccc2[nH]nc(-c3cnc(N4CC(C)(CN5CCCC5)C4)c(F)c3)c2c1F)c1c(Cl)cncc1Cl] | (R)-5-(1-(3,5- dichloropyridin-4- yl)ethoxy)-4-fluoro- 3-(5-fluoro-6-(3- methyl-3- (pyrrolidin-1- ylmethyl)azetidin-1- yl)pyridin-3-yl)-1H- indazole | LCMS m/z = 573.2 (M + H); 1HNMR (400 MHz, DMSO-d6) δ 13.43 (brs, 1H), 8.61 (s, 2H), 8.31 (d, J = 1.2 Hz, 1H), 7.73 (d, J = 13.6 Hz, 1H), 7.27 (d, J = 8.8 Hz, 1H), 7.12 (t, J = 7.6 Hz, 1H), 5.99 (q, J = 6.8 Hz, 1H), 3.92 (d, J = 7.2 Hz, 2H), 3.80 (dd, J = 8.0 Hz, 2.0 Hz, 2H), 2.62 (s, 2H), 2.48-2.41 (m, 4H), 1.79 (d, J = 6.4 Hz, 3H), 1.72-1.63 (m, 4H), 1.37 (s, 3H) |
| Example 186. | 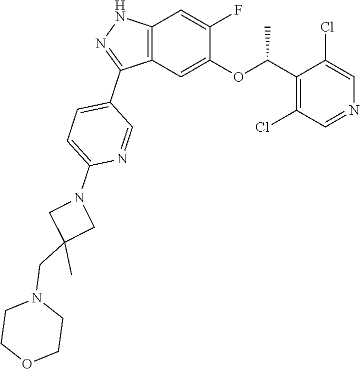 [SMILES: C[C@@H](Oc1cc2c(-c3ccc(N4CC(C)(CN5CCOCC5)C4)nc3)n[nH]c2cc1F)c1c(Cl)cncc1Cl] | (R)-4-((1-(5-(5-(1- (3,5-dichloro- pyridin-4-yl) ethoxy)-6-fluoro- 1H-indazol-3- yl)pyridin-2-yl)-3- methylazetidin-3- yl)methyl)morpho- line | LCMS m/z = 571.3 (M + H); 1HNMR (400 MHz, DMSO-d6) δ 8.63 (s, 2H), 8.46 (d, J = 2.0 Hz, 1H), 7.79 (dd, J = 8.4 Hz, 2.0 Hz, 1H), 7.43 (d, J = 10.8 Hz, 1H), 7.22 (d, J = 8.0 Hz, 1H), 6.50 (d, J = 8.8 Hz, 1H), 6.13 (q, J = 6.0 Hz, 1H), 3.77 (d, J = 7.6 Hz, 2H), 3.70 (d, J = 8.0 Hz, 2H), 3.58 (t, J = 4.4 Hz, 4H), 2.57-2.54 (m, 2H), 2.37- 2.36 (m, 4H), 1.80 (d, J = 6.8 Hz, 3H), 1.38 (s, 3H) |
| Example 187. | 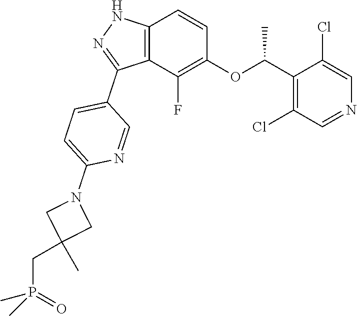 [SMILES: C[C@@H](Oc1ccc2[nH]nc(-c3ccc(N4CC(C)(CP(C)(C)=O)C4)nc3)c2c1F)c1c(Cl)cncc1Cl] | (R)-((1-(5-(5-(1- (3,5-dichloro- pyridin-4-yl) ethoxy)-4-fluoro-1H-indazol-3- yl)pyridin-2-yl)-3- methylazetidin-3- yl)methyl)dimethyl- phosphine oxide | LCMS m/z = 562.2 (M + H); 1HNMR (400 MHz, DMSO-d6) δ 13.32 (s, 1H), 8.61 (s, 2H), 8.45 (t, J = 1.6 Hz, 1H), 7.87 (dd, J = 8.8 Hz, 2.0 Hz, 1H), 7.24 (d, J = 6.0 Hz, 1H), 7.11 (dd, J = 8.8 Hz, 7.6 Hz, 1H), 6.48 (d, J = 8.4 Hz, 1H), 5.98 (q, J = 6.8 Hz, 1H), 3.95-3.93 (m, 2H), 3.76-3.74 (m, 2H), 2.18 (d, J = 11.2 Hz, 2H), 1.79 (d, J = 6.8 Hz, 3H), 1.55 (s, 3H), 1.45 (s, 3H), 1.42 (s, 3H) |
| Example 188. | 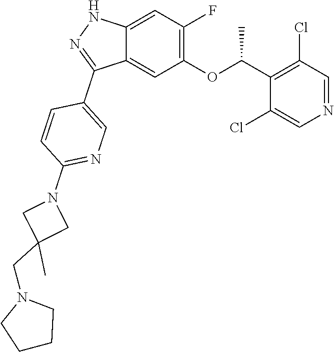 [SMILES: C[C@@H](Oc1cc2c(-c3ccc(N4CC(C)(CN5CCCC5)C4)nc3)n[nH]c2cc1F)c1c(Cl)cncc1Cl] | (R)-5-(1-(3,5- dichloropyridin-4- yl)ethoxy)-6-fluoro- 3-(6-(3-methyl-3- (pyrrolidin-1- ylmethyl)azetidin-1- yl)pyridin-3-yl)-1H- indazole | LCMS m/z = 555.3 (M + H); 1HNMR (400 MHz, DMSO-d6) δ 13.05 (s, 1H), 8.63 (s, 2H), 8.46 (d, J = 1.6 Hz, 1H), 7.78 (dd, J = 8.8 Hz, 2.4 Hz, 1H), 7.43 (d, J = 11.2 Hz, 1H), 7.23 (d, J = 8.0 Hz, 1H), 6.50 (d, J = 8.2 Hz, 1H), 6.13 (q, J = 6.8 Hz, 1H), 3.78 (d, J = 8.0 Hz, 2H), 3.69 (d, J = 7.6 Hz, 2H), 2.62 (s, 2H), 2.47-2.33 (m, 4H), 1.80 (d, J = 6.4 Hz, 3H), 1.72-1.69 (m, 4H), 1.37 (s, 3H) |
| Example 189. | 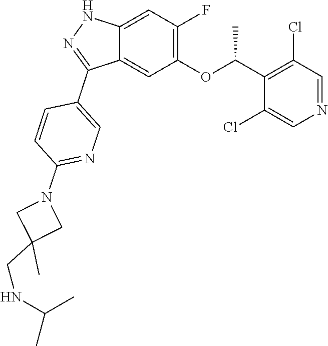 [SMILES: CC(C)NCC1(C)CN(c2ccc(-c3n[nH]c4cc(F)c(O[C@H](C)c5c(Cl)cncc5Cl)cc34)cn2)C1] | (R)-N-((1-(5-(5-(1- (3,5-dichloro- pyridin-4-yl) ethoxy)-6-fluoro- 1H-indazol-3- yl)pyridin-2-yl)-3- methylazetidin-3- yl)methyl)propan-2- amine | LCMS m/z = 543.2 (M + H); 1HNMR (400 MHz, DMSO-d6) δ 13.04 (s, 1H), 8.63 (s, 2H), 8.46 (d, J = 2.0 Hz, 1H), 7.78 (dd, J = 8.8 Hz, 2.4 Hz, 1H), 7.43 (d, J = 10.8 Hz, 1H), 7.23 (d, J = 8.0 Hz, 1H), 6.47 (d, J = 8.8 Hz, 1H), 6.13 (q, J = 6.4 Hz, 1H), 3.82 (d, J = 8.0 Hz, 2H), 3.60 (d, J = 7.6 Hz, 2H), 2.73- 2.71 (m, 1H), 2.66 (s, 2H), 1.80 (d, J = 7.2 Hz, 3H), 1.30 (s, 3H), 1.01 (d, J = 6.4 Hz, 6H) |
| Example 190. | 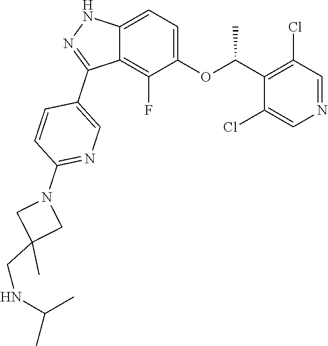 [SMILES: CC(C)NCC1(C)CN(c2ccc(-c3n[nH]c4ccc(O[C@H](C)c5c(Cl)cncc5Cl)c(F)c34)cn2)C1] | (R)-N-((1-(5-(5-(1- (3,5-dichloro- pyridin- 4-yl)ethoxy)-4- fluoro-1H- indazol-3- yl)pyridin-2-yl)-3- methylazetidin-3- yl)methyl)propan-2- amine | LCMS m/z = 543.3 (M + H); 1HNMR (400 MHz, DMSO-d6) δ 13.32 (brs, 1H), 8.61 (s, 2H), 8.44 (s, 1H), 7.88-7.85 (m, 1H), 7.24 (d, J = 8.8 Hz, 1H), 7.11 (t, J = 7.2 Hz, 1H), 6.46 (d, J = 8.8 Hz, 1H), 5.98 (q, J = 6.4 Hz, 1H), 3.83 (d, J = 8.0 Hz, 2H), 3.61 (d, J = 8.0 Hz, 2H), 2.73- 2.65 (m, 3H), 1.79 (d, J = 6.4 Hz, 3H), 1.31 (s, 3H), 1.05 (s, 6H) |
| Example 191. | 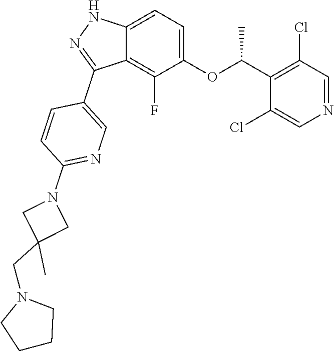 [SMILES: C[C@@H](Oc1ccc2[nH]nc(-c3ccc(N4CC(C)(CN5CCCC5)C4)nc3)c2c1F)c1c(Cl)cncc1Cl] | (R)-5-(1-(3,5- dichloropyridin-4- yl)ethoxy)-4-fluoro- 3-(6-(3-methyl-3- (pyrrolidin-1- ylmethyl)azetidin-1- yl)pyridin-3-yl)-1H- indazole | LCMS m/z = 555.3 (M + H); 1HNMR (400 MHz, DMSO-d6) δ 13.31 (s, 1H), 8.61 (s, 2H), 8.44 (s, 1H), 7.88-7.85 (m, 1H), 7.24 (d, J = 8.8 Hz, 1H), 7.11 (t, J = 7.2 Hz, 1H), 6.47 (d, J = 8.4 Hz, 1H), 5.98 (q, J = 6.4 Hz, 1H), 3.76 (d, J = 7.6 Hz, 2H), 3.67 (d, J = 8.0 Hz, 2H), 2.61 (s, 2H), 2.46-2.43 (m, 4H), 1.79 (d, J = 6.4 Hz, 3H), 1.69-1.67 (m, 4H), 1.36 (s, 3H) |
| Example 192. | 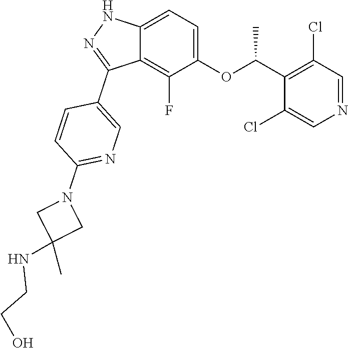 [SMILES: C[C@@H](Oc1ccc2[nH]nc(-c3ccc(N4CC(C)(NCCO)C4)nc3)c2c1F)c1c(Cl)cncc1Cl] | (R)-2-((1-(5-(5-(1- (3,5-dichloro- pyridin-4-yl) ethoxy)-4- fluoro-1H-inda- zol-3- yl)pyridin-2-yl)-3- methylazetidin-3- yl)amino)ethanol | LCMS m/z = 531.2 (M + H); 1HNMR (400 MHz, DMSO-d6) δ 13.31 (brs, 1H), 8.61 (s, 2H), 8.45 (t, J = 2.4 Hz, 1H), 7.87 (dt, J = 8.8 Hz, 2.0 Hz, 1H), 7.24 (d, J = 9.2 Hz, 1H), 7.11 (t, J = 7.6 Hz, 1H), 6.50 (d, J = 8.8 Hz, 1H), 5.98 (q, J = 7.2 Hz, 1H), 4.54 (brs, 1H), 3.82 (d, J = 8.0 Hz, 2H), 3.75 (d, J = 8.0 Hz, 2H), 3.47 (q, J = 5.6 Hz, 2H), 2.62 (t, J = 6.0 Hz 2H), 1.79 (d, J = 6.8 Hz, 3H), 1.42 (s, 3H) |
| Example 193. | 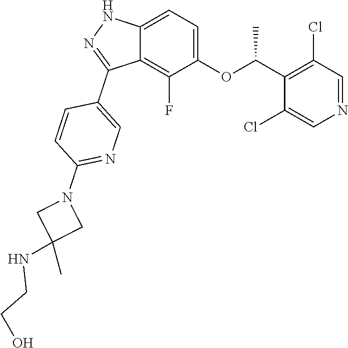 | (R)-((1-(5-(5-(1- (3,5-dichloro-pyridin- 4-yl)ethoxy)-6- fluoro-1H- indazol-3- yl)-3-fluoropyridin- 2-yl)-3- methylazetidin-3- yl)methyl)dimethyl- phosphine oxide | LCMS m/z = 580.2 (M + H); 1HNMR (400 MHz, DMSO-d6) δ 13.18 (s, 1H), 8.60 (s, 2H), 8.34 (s, 1H), 7.68 (dd, J = 13.2 Hz, 1.6 Hz, 1H), 7.45 (d, J = 10.8 Hz, 1H), 7.28 (d, J = 7.6 Hz, 1H), 6.17 (q, J = 6.8 Hz, 1H), 4.12 (d, J = 7.6 Hz, 2H), 3.87 (d, J = 7.6 Hz, 2H), 2.20 (d, J = 11.2 Hz, 2H), 1.80 (d, J = 6.8 Hz, 3H), 1.56 (s, 3H), 1.46 (s, 3H), 1.43 (s, 3H) |
| Example 194. | 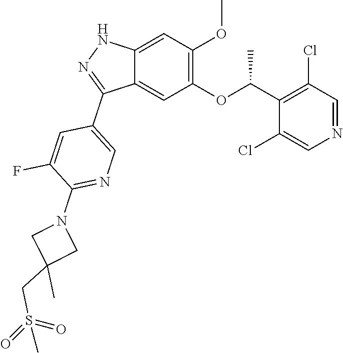 [SMILES: COc1cc2[nH]nc(-c3cnc(N4CC(C)(CS(C)(=O)=O)C4)c(F)c3)c2cc1O[C@H](C)c1c(Cl)cncc1Cl] | (R)-5-(1-(3,5- dichloropyridin-4- yl)ethoxy)-3-(5- fluoro-6-(3-methyl- 3-((methyl- sulfonyl)meth- yl)azetidin-1- yl)pyridin-3-yl)-6- methoxy-1H- indazole | LCMS m/z = 594.2 (M + H); 1HNMR (400 MHz, DMSO-d6) δ 12.93 (brs, 1H), 8.59 (s, 2H), 8.31 (t, J = 1.2 Hz, 1H), 7.66 (dd, J = 13.2 Hz, 1.6 Hz, 1H), 7.08 (s, 1H), 7.00 (s, 1H), 5.99 (q, J = 6.4 Hz, 1H), 4.21 (d, J = 8.0 Hz, 2H), 3.89-3.86 (m, 5H), 3.67 (s, 2H), 3.03 (s, 3H), 1.76 (d, J = 6.4 Hz, 3H), 1.61 (s, 3H) |
| Example 195. | 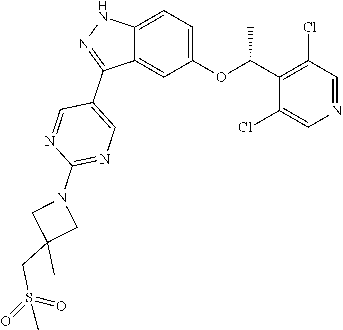 [SMILES: C[C@@H](Oc1ccc2[nH]nc(-c3cnc(N4CC(C)(CS(C)(=O)=O)C4)nc3)c2c1)c1c(Cl)cncc1Cl] | Name (R)-5-(1- (3,5-dichloro- pyridin- 4-yl)ethoxy)-3-(2- (3-methyl-3- ((methylsulfonyl)- methyl)azetidin-1- yl)pyrimidin-5-yl)- 1H-indazole | LCMS m/z = 547.2 (M + H); 1HNMR (400 MHz, DMSO-d6) δ 13.13 (s, 1H), 8.77 (s, 2H), 8.57 (s, 2H), 7.47 (d, J = 8.8 Hz, 1H), 7.25 (d, J = 2.0 Hz, 1H), 7.10 (dd, J = 8.8 Hz, 2.0 Hz, 1H), 6.15 (q, J = 6.4 Hz, 1H), 4.18 (d, J = 8.8 Hz, 2H), 3.85 (d, J = 8.8 Hz, 2H), 3.68 (s, 2H), 3.03 (s, 3H), 1.75 (d, J = 6.4 Hz, 3H), 1.60 (s, 3H) |
| Example 196. | 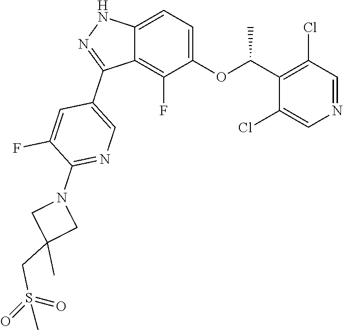 [SMILES: C[C@@H](Oc1ccc2[nH]nc(-c3cnc(N4CC(C)(CS(C)(=O)=O)C4)c(F)c3)c2c1F)c1c(Cl)cncc1Cl] | (R)-5-(1-(3,5- dichloropyridin-4- yl)ethoxy)-4-fluoro- 3-(5-fluoro-6-(3- methyl-3-((methyl- sulfonyl)meth- yl)azetidin-1- yl)pyridin-3-yl)-1H- indazole | LCMS m/z= 581.2 (M + H); 1HNMR (400 MHz, DMSO-d6) δ 13.45 (brs, 1H), 8.61 (s, 2H), 8.33 (d, J = 1.6 Hz, 1H), 7.75 (d, J = 13.2 Hz, 1H), 7.27 (d, J = 8.8 Hz, 1H), 7.13 (t, J = 7.6 Hz, 1H), 5.99 (q, J = 6.8 Hz, 1H), 4.21 (d, J = 8.0 Hz, 2H), 3.87 (d, J = 8.0 Hz, 2H), 3.66 (s, 2H), 3.02 (s, 3H), 1.79 (d, J = 7.2 Hz, 3H), 1.61 (s, 3H) |
| Example 197. | 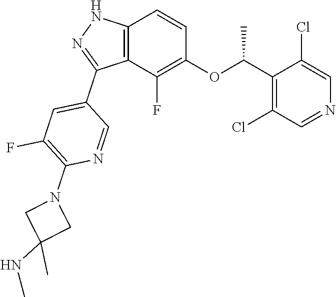 [SMILES: CNC1(C)CN(c2ncc(-c3n[nH]c4ccc(O[C@H](C)c5c(Cl)cncc5Cl)c(F)c34)cc2F)C1] | (R)-1-(5-(5-(1-(3,5- dichloropyridin-4- yl)ethoxy)-4-fluoro- 1H-indazol-3-yl)-3- fluoropyridin-2-yl)- N,3- dimethylazetidin-3- amine | LCMS m/z = 519.1 (M + H); 1HNMR (400 MHz, DMSO-d6) δ 13.44 (brs, 1H), 8.61 (s, 2H), 8.34-8.29 (m, 1H), 7.73 (dt, J = 13.2 Hz, 1.6 Hz, 1H), 7.27 (d, J = 8.8 Hz, 1H), 7.12 (dd, J = 9.2 Hz, 7.6 Hz, 1H), 5.99 (q, J = 6.4 Hz, 1H), 3.97 (d, J = 8.0 Hz, 2H), 3.84 (dd, J = 8.4 Hz, 1.6 Hz, 2H), 2.24 (s, 3H), 1.79 (d, J = 6.8 Hz, 3H), 1.39 (s, 3H) |
| Example 198. | 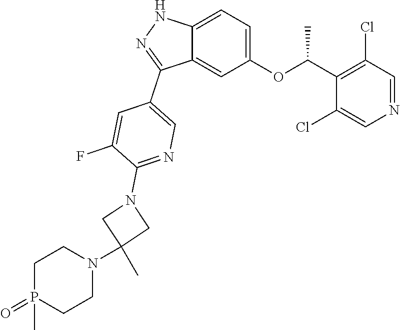 [SMILES: C[C@@H](Oc1ccc2[nH]nc(-c3cnc(N4CC(C)(N5CCP(C)(=O)CC5)C4)c(F)c3)c2c1)c1c(Cl)cncc1Cl] | (R)-1-(1-(5-(5-(1- (3,5-dichloropyridin- 4-yl)ethoxy)-1H- indazol-3-yl)-3- fluoropyridin-2-yl)- 3-methylazetidin-3- yl)-4-methyl-1,4- azaphosphinane 4- oxide | LCMS m/z = 603 (M + H); 1HNMR (400 MHz, DMSO-d6) δ 13.12 (s, 1H), 8.57 (s, 2H), 8.37 (s, 1H), 7.72 (dd, J = 13.2 Hz, 1.6 Hz, 1H), 7.48 (d, J = 9.2 Hz, 1H), 7.20 (d, J = 1.6 Hz, 1H), 7.11 (dd, J = 9.2 Hz, 2.0 Hz, 1H), 6.14 (q, J = 6.4 Hz, 1H), 3.93 (d, J = 7.6 Hz, 2H), 3.84 (d, J = 6.4 Hz, 2H), 2.79- 2.69 (m, 2H), 2.55-2.52 (m, 2H), 1.91-1.75 (m, 7H), 1.42 (d, J = 13.2 Hz, 3H), 1.35 (s, 3H) |
| Example 199. | 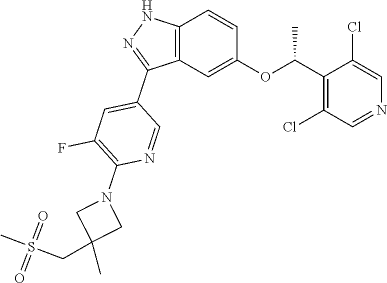 [SMILES: C[C@@H](Oc1ccc2[nH]nc(-c3cnc(N4CC(C)(CS(C)(=O)=O)C4)c(F)c3)c2c1)c1c(Cl)cncc1Cl] | (R)-5-(1-(3,5- dichloropyridin-4- yl)ethoxy)-3-(5- fluoro-6-(3-methyl- 3-((methyl- sulfonyl)meth- yl)azetidin-1- yl)pyridin-3-yl)-1H- indazole | LCMS m/z = 564.3 (M + H); 1HNMR (400 MHz, DMSO-d6) δ 13.14 (s, 1H), 8.58 (s, 2H), 8.38 (t, J = 1.6 Hz, 1H), 7.72 (dd, J = 13.2, 1.6 Hz, 1H), 7.48 (d, J = 9.2 Hz, 1H), 7.20 (d, J = 1.6 Hz, 1H), 7.11 (dd, J = 8.0, 2.0 Hz, 1H), 6.15 (q, J = 7.2 Hz, 1H), 4.22 (d, J = 8.4 Hz, 2H), 3.88 (dd, J = 8.4, 2.0 Hz, 2H), 3.68 (s, 2H), 3.04 (s, 3H), 1.76 (d, J = 6.8 Hz, 3H), 1.62 (s, 3H) |
| Example 200. | 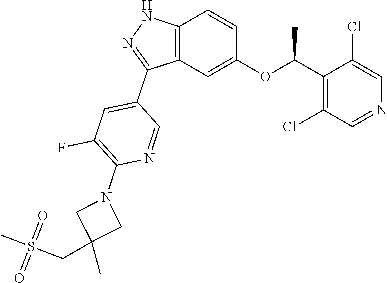 [SMILES: C[C@H](Oc1ccc2[nH]nc(-c3cnc(N4CC(C)(CS(C)(=O)=O)C4)c(F)c3)c2c1)c1c(Cl)cncc1Cl] | (S)-5-(1-(3,5- dichloropyridin-4- yl)ethoxy)-3-(5- fluoro-6-(3-methyl- 3-((methyl- sulfonyl)meth- yl)azetidin-1- yl)pyridin-3-yl)-1H- indazole | LCMS m/z = 564.2 (M + H); 1HNMR (400 MHz, DMSO-d6) δ 13.12 (brs, 1H), 8.57 (s, 2H), 8.38 (t, J = 1.6 Hz, 1H), 7.74 (dd, J = 13.2 Hz, 2.0 Hz, 1H), 7.49 (d, J = 8.8 Hz, 1H), 7.20 (d, J = 2.4 Hz, 1H), 7.12 (dd, J = 8.8 Hz, 2.4 Hz, 1H), 6.17 (q, J = 6.4 Hz, 1H), 4.23 (d, J = 8.0 Hz, 2H), 3.90 (dd, J = 8.0 Hz, 1.6 Hz, 2H), 3.67 (s, 2H), 3.03 (s, 3H), 1.77 (d, J = 6.4 Hz, 3H), 1.62 (s, 3H) |
| Example 201. | 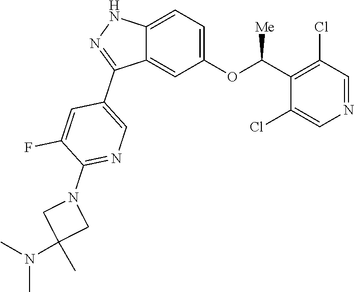 [SMILES: C[C@H](Oc1ccc2[nH]nc(-c3cnc(N4CC(C)(N(C)C)C4)c(F)c3)c2c1)c1c(Cl)cncc1Cl] | 1-[5-[5-[(1S)-1-(3,5- dichloro-4- pyridyl)ethoxy]-1H- indazol-3-yl]-3- fluoro-2-pyridyl]- N,N,3-trimethyl- azetidin-3-amine | LCMS m/z = 515.1 (M+H); 1HNMR (400 MHz, DMSO-d6) δ 11.67 (br s, 1H), 8.57 (s, 2H), 8.41 (t, J = 1.6 Hz, 1H), 7.81 (dd, J = 1.8, 13.1 Hz, 1H), 7.49 (d, J = 9.0 Hz, 1H), 7.21 (d, J = 2.2 Hz, 1H), 7.11 (dd, J = 2.3, 9.0 Hz, 1H), 6.18-6.12 (m, 1H), 4.50 (d, J = 9.2 Hz, 2H), 4.04 (br s, 2H), 2.72 (d, J = 4.8 Hz, 6H), 1.77 (d, J = 6.6 Hz, 3H), 1.69 (s, 3H) |
| Example 202. | 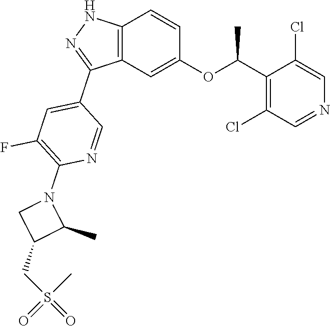 [SMILES: C[C@H](Oc1ccc2[nH]nc(-c3cnc(N4C[C@@H](CS(C)(=O)=O)[C@@H]4C)c(F)c3)c2c1)c1c(Cl)cncc1Cl] | 5-((S)-1-(3,5- dichloropyridin-4- yl)ethoxy)-3-(5- fluoro-6-((2S,3R)-2- methyl-3-((meth- ylsulfonyl)meth- yl)azetidin-1- yl)pyridin-3-yl)-1H- indazole | LCMS m/z = 564.1 (M + H); 1HNMR (400 MHz, DMSO-d6) δ 13.13 (s, 1H), 8.57 (s, 2H), 8.41 (t, J = 4.8 Hz, 1H), 7.74 (dd, J = 13.2 Hz, 1.6 Hz, 1H), 7.49 (d, J = 9.2 Hz, 1H), 7.20 (d, J = 1.2 Hz, 1H), 7.11 (dd, J = 8.8 Hz, 2.4 Hz, 1H), 6.17 (q, J = 6.4 Hz, 1H), 4.38-4.33 (m, 1H), 4.32-4.28 (m, 1H), 3.90- 3.86 (m, 1H), 3.58-3.54 (m, 2H), 3.00 (s, 3H), 2.89-2.85 (m, 1H), 1.77 (d, J = 6.4 Hz, 3H), 1.56 (d, J = 6.0 Hz, 3H) |
| Example 203. | 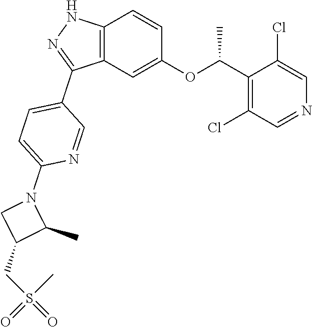 [SMILES: C[C@@H](Oc1ccc2[nH]nc(-c3ccc(N4C[C@@H](CS(C)(=O)=O)[C@@H]4C)nc3)c2c1)c1c(Cl)cncc1Cl] | 5-((R)-1-(3,5- dichloropyridin-4- yl)ethoxy)-3-(6- ((2S,3R)-2-methyl- 3-((methyl- sulfonyl)meth- yl)azetidin-1- yl)pyridin-3-yl)-1H- indazole | LCMS m/z = 546.1 (M+H); 1HNMR (400 MHz, DMSO-d6) δ 13.01 (s, 1H), 8.60 (s, 2H), 8.50 (d, J = 2.0 Hz, 1H), 7.85 (dd, J = 8.4 Hz, 2.4 Hz, 1H), 7.46 (d, J = 8.8 Hz, 1H), 7.16 (d, J = 1.6 Hz, 1H), 7.09 (d, J = 8.8 Hz, 2.4 Hz 1H), 6.56 (d, J = 8.8 Hz, 1H), 6.11 (q, J = 6.8 Hz, 1H), 4.20-4.14 (m, 2H), 3.69 (t, J = 7.2 Hz, 1H), 3.56-3.53 (m, 2H), 3.00 (s, 3H), 2.89-2.80 (m, 1H), 1.76 (d, J = 6.4 Hz, 3H), 1.55 (d, J = 6.4 Hz, 3H) |
| Example 204. | 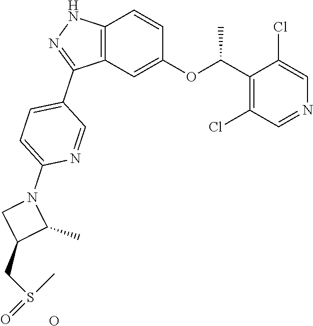 | 5-((R)-1-(3,5- dichloropyridin-4- yl)ethoxy)-3-(6- ((2R,3S)-2-methyl- 3-((methyl- sulfonyl)meth- yl)azetidin-1- yl)pyridin-3-yl)-1H- indazole | LCMS m/z = 546.2 (M + H); 1HNMR (400 MHz, DMSO-d6) δ 13.01 (s, 1H), 8.60 (s, 2H), 8.52 (d, J = 2.0 Hz, 1H), 7.85 (dd, J = 8.8 Hz, 2.4 Hz, 1H), 7.46 (d, J = 8.8 Hz, 1H), 7.16 (d, J = 1.6 Hz, 1H), 7.09 (dd, J = 9.2 Hz, 2.4 Hz, 1H), 6.56 (d, J = 8.8 Hz, 1H), 6.11 (q, J = 6.4 Hz, 1H), 4.20-4.15 (m, 2H), 3.68 (t, J = 7.2 Hz, 1H), 3.56- 3.53 (m, 2H), 3.00 (s, 3H), 2.87-2.82 (m, 1H), 1.76 (d, J = 6.4 Hz, 3H), 1.55 (d, J = 6.0 Hz, 3H) |
| Example 205. | 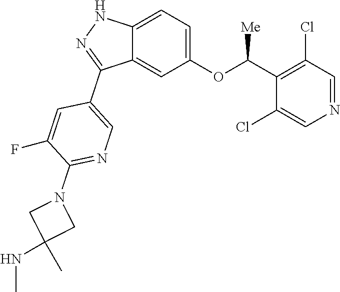 [SMILES: CNC1(C)CN(c2ncc(-c3n[nH]c4ccc(O[C@@H](C)c5c(Cl)cncc5Cl)cc34)cc2F)C1] | 1-[5-[5-[(1S)-1-(3,5- dichloro-4- pyridyl)ethoxy]-1H- indazol-3-yl]-3- fluoro-2-pyridyl]- N,3-dimethyl- azetidin-3-amine | LCMS m/z = 501.1 (M + H); 1HNMR (400 MHz, DMSO-d6) δ 13.10 (br s, 1H), 8.57 (s, 2H), 8.36 (t, J = 1.7 Hz, 1H), 7.69 (dd, J = 1.8, 13.3 Hz, 1H), 7.47 (d, J = 9.2 Hz, 1H), 7.20 (d, J = 2.1 Hz, 1H), 7.10 (dd, J = 2.3, 9.0 Hz, 1H), 6.14 (q, J = 6.7 Hz, 1H), 4.06-3.95 (m, 2H), 3.85 (dd, J = 2.1, 8.4 Hz, 2H), 2.33 (br d, J = 1.7 Hz, 1H), 2.25 (s, 3H), 1.76 (d, J = 6.7 Hz, 3H), 1.40 (s, 3H) |
| Example 206. | 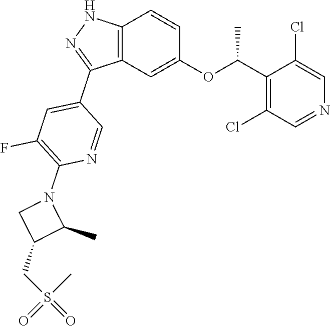 [SMILES: C[C@@H](Oc1ccc2[nH]nc(-c3cnc(N4C[C@@H](CS(C)(=O)=O)[C@@H]4C)c(F)c3)c2c1)c1c(Cl)cncc1Cl] | 5-((R)-1-(3,5- dichloropyridin-4- yl)ethoxy)-3-(5- fluoro-6-((2S,3R)-2- methyl-3-((meth- ylsulfonyl)meth- yl)azetidin-1- yl)pyridin-3-yl)-1H- indazole | LCMS m/z = 564.1 (M + H); 1HNMR (400 MHz, DMSO-d6) δ 13.12 (s, 1H), 8.58 (s, 2H), 8.37 (t, J = 1.6 Hz, 1H), 7.74 (dd, J = 13.2 Hz, 2.0 Hz, 1H), 7.49 (d, J = 9.2 Hz, 1H), 7.19 (d, J = 1.6 Hz, 1H), 7.11 (dd, J = 8.8 Hz, 2.4 Hz, 1H), 6.15 (q, J = 6.8 Hz, 1H), 4.39-4.28 (m, 2H), 3.91-3.86 (m, 1H), 3.61- 3.50 (m, 2H), 3.00 (s, 3H), 2.89-2.83 (m, 1H), 1.77 (d, J = 6.8 Hz, 3H), 1.54 (d, J = 6.0 Hz, 3H) |
| Example 207. | 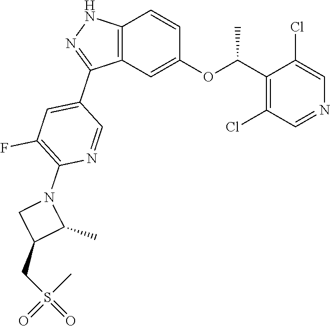 [SMILES: C[C@@H]1[C@@H](CS(C)(=O)=O)CN1c1ncc(-c2n[nH]c3ccc(O[C@H](C)c4c(Cl)cncc4Cl)cc23)cc1F] | 5-((R)-1-(3,5- dichloropyridin-4- yl)ethoxy)-3-(5- fluoro-6-((2R,3S)-2- methyl-3-((meth- ylsulfonyl)meth- yl)azetidin-1- yl)pyridin-3-yl)-1H- indazole | LCMS m/z = 564.2 (M + H); 1HNMR (400 MHz, DMSO-d6) δ 13.12 (brs, 1H), 8.57 (s, 2H), 8.39 (t, J = 1.6 Hz, 1H), 7.72 (dd, J = 13.2 Hz, 1.6 Hz, 1H), 7.48 (d, J = 9.2 Hz, 1H), 7.20 (d, J = 2.0 Hz, 1H), 7.10 (dd, J = 9.2 Hz, 2.8 Hz, 1H), 6.14 (q, J = 6.4 Hz, 1H), 4.38-4.29 (m, 2H), 3.89-3.86 (m, 1H), 3.61- 3.51 (m, 2H), 3.00 (s, 3H), 2.88-2.83 (m, 1H), 1.76 (d, J = 6.4 Hz, 3H), 1.53 (d, J = 6.4 Hz, 3H) |
| Example 208. | 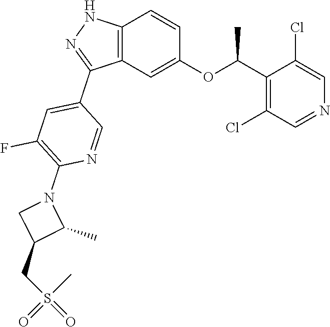 [SMILES: C[C@H](Oc1ccc2[nH]nc(-c3cnc(N4C[C@H](CS(C)(=O)=O)[C@H]4C)c(F)c3)c2c1)c1c(Cl)cncc1Cl] | 5-((S)-1-(3,5- dichloropyridin-4- yl)ethoxy)-3-(5- fluoro-6-((2R,3S)-2- methyl-3-((meth- ylsulfonyl)meth- yl)azetidin-1- yl)pyridin-3-yl)-1H- indazole | LCMS m/z = 564.2 (M + H); 1HNMR (400 MHz, DMSO-d6) δ 13.13 (brs, 1H), 8.57 (s, 2H), 8.37 (t, J = 3.2 Hz, 1H), 7.74 (dd, J = 13.2 Hz, 1.6 Hz, 1H), 7.49 (d, J = 9.2 Hz, 1H), 7.19 (s, 1H), 7.11 (dd, J = 8.8 Hz, 2.4 Hz, 1H), 6.16 (q, J = 6.4 Hz, 1H), 4.40-4.29 (m, 2H), 3.91- 3.86 (m, 1H), 3.56-3.50 (m, 2H), 3.00 (s, 3H), 2.90-2.81 (m, 1H), 1.76 (d, J = 6.4 Hz, 3H), 1.53 (d, J = 6.0 Hz, 3H) |
| Example 209. | 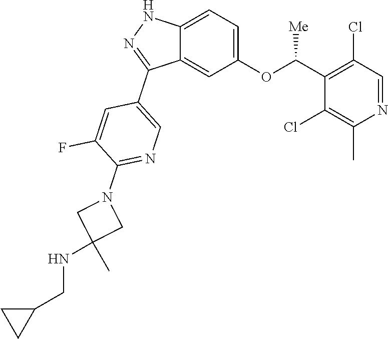 [SMILES: Cc1ncc(Cl)c([C@@H](C)Oc2ccc3[nH]nc(-c4cnc(N5CC(C)(NCC6CC6)C5)c(F)c4)c3c2)c1Cl] | N-(cyclo- propylmethyl)- 1-[5-[5-[(1R)-1- (3,5-dichloro-2- methyl-4- pyridyl)ethoxy]-1H- indazol-3-yl]-3- fluoro-2-pyridyl]-3- methyl-azetidin-3- amine | LCMS m/z = 555.2 (M + H); 1HNMR (400 MHz, DMSO-d6) δ 13.11 (s, 1H), 8.42 (s, 1H), 8.24 (s, 1H), 7.66 (dd, J = 1.8, 13.2 Hz, 1H), 7.47 (d, J = 9.0 Hz, 1H), 7.12-7.04 (m, 2H), 6.11 (q, J = 6.6 Hz, 1H), 3.99 (br d, J = 8.1 Hz, 2H), 3.84 (br d, J = 7.0 Hz, 2H), 2.58 (s, 3H), 2.39 (d, J = 6.7 Hz, 2H), 2.29 (br s, 1H), 1.75 (d, J = 6.6 Hz, 3H), 1.42 (s, 3H), 0.89-0.81 (m, 1H), 0.43-0.38 (m, 2H), 0.15-0.11 (m, 2H) |
| Example 210. | 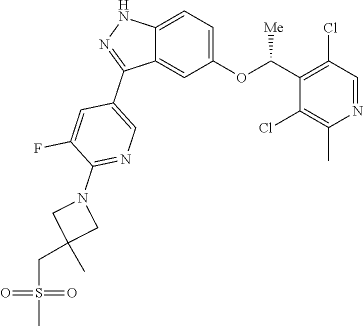 [SMILES: Cc1ncc(Cl)c([C@@H](C)Oc2ccc3[nH]nc(-c4cnc(N5CC(C)(CS(C)(=O)=O)C5)c(F)c4)c3c2)c1Cl] | 5-[(1R)-1-(3,5- dichloro-2-methyl-4-pyridyl)eth oxy]-3-[5-fluoro- 6-[3-methyl-3- (methylsulfonyl- methyl)azetidin- 1-yl]-3- pyridyl]-1H- indazole | LCMS m/z = 578.1 (M + H); 1HNMR (400 MHz, DMSO-d6) δ 13.13 (br s, 1H), 8.42 (s, 1H), 8.26 (s, 1H), 7.69 (dd, J = 1.8, 13.1 Hz, 1H), 7.48 (d, J = 9.0 Hz, 1H), 7.10 (dd, J = 2.3, 9.0 Hz, 1H), 7.05 (d, J = 1.8 Hz, 1H), 6.11 (q, J = 6.7 Hz, 1H), 4.21 (br d, J = 8.3 Hz, 2H), 3.88 (d, J = 7.5 Hz, 2H), 3.67 (s, 2H), 3.03 (s, 3H), 2.58 (s, 3H), 1.75 (d, J = 6.6 Hz, 3H), 1.61 (s, 3H) |
| Example 211. | 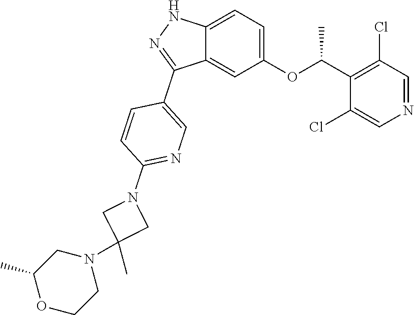 [SMILES: C[C@@H]1CN(C2(C)CN(c3ccc(-c4n[nH]c5ccc(O[C@H](C)c6c(Cl)cncc6Cl)cc45)cn3)C2)CCO1] | (R)-4-(1-(5-(5-((R)- 1-(3,5- dichloropyridin-4- yl)ethoxy)-1H- indazol-3-yl)pyridin- 2-yl)-3- methylazetidin-3- yl)-2- methylmorpholine | LCMS m/z = 555.3 (M + H); 1HNMR (400 MHz, DMSO-d6) δ 12.99 (s, 1H), 8.59 (s, 2H), 8.51 (d, J = 2.0 Hz, 1H), 7.87 (dd, J = 8.4 Hz, 2.0 Hz, 1H), 7.47 (d, J = 8.8 Hz, 1H), 7.17 (d, J = 2.0 Hz, 1H), 7.10 (dd, J = 8.8 Hz, 2.0 Hz, 1H), 6.53 (d, J = 8.8 Hz, 1H), 6.14 (q, J = 6.8 Hz, 1H), 3.83-3.78 (m, 3H), 3.70 (d, J = 8.0 Hz, 2H), 3.54- 3.45 (m, 2H), 2.65-2.63 (m, 1H), 2.55-2.50 (m, 1H), 2.26- 2.20 (m, 1H), 1.94 (t, J = 10.4 Hz, 1H), 1.77 (d, J = 6.8 Hz, 3H), 1.33 (s, 3H), 1.08 (d, J = 6.4 Hz, 3H) |
| Example 212. | 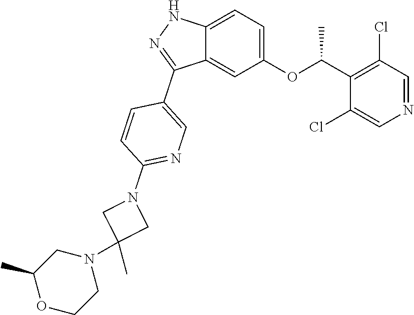 [SMILES: C[C@H]1CN(C2(C)CN(c3ccc(-c4n[nH]c5ccc(O[C@H](C)c6c(Cl)cncc6Cl)cc45)cn3)C2)CCO1] | (S)-4-(1-(5-(5-((R)- 1-(3,5- dichloropyridin-4- yl)ethoxy)-1H- indazol-3-yl)pyridin- 2-yl)-3- methylazetidin-3- yl)-2- methylmorpholine | LCMS m/z = 555.3 (M + H); 1HNMR (400 MHz, DMSO-d6) δ 12.99 (s, 1H), 8.59 (s, 2H), 8.51 (d, J = 2.0 Hz, 1H), 7.85 (dd, J = 8.4 Hz, 2.4 Hz, 1H), 7.46 (d, J = 9.2 Hz, 1H), 7.16 (d, J = 1.6 Hz, 1H), 7.09 (dd, J = 8.8 Hz, 2.4 Hz, 1H), 6.52 (d, J = 8.4 Hz, 1H), 6.10 (q, J = 6.4 Hz, 1H), 3.86-3.75 (m, 3H), 3.69 (d, J = 8.0 Hz, 2H), 3.55- 3.43 (m, 2H), 2.68-2.57 (m, 1H), 2.55-2.52 (m, 1H), 2.28- 2.17 (m, 1H), 1.91 (t, J = 10.4 Hz, 1H), 1.75 (d, J = 6.4 Hz, 3H), 1.32 (s, 3H), 1.07 (d, J = 6.0 Hz, 3H) |
| Example 213. | 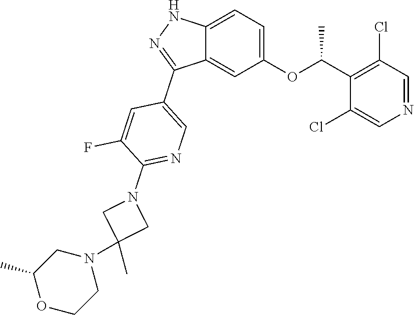 [SMILES: C[C@@H]1CN(C2(C)CN(c3ncc(-c4n[nH]c5ccc(O[C@H](C)c6c(Cl)cncc6Cl)cc45)cc3F)C2)CCO1] | (R)-4-(1-(5-(5-((R)- 1-(3,5- dichloropyridin-4- yl)ethoxy)-1H- indazol-3-yl)-3- fluoropyridin-2-yl)- 3-methylazetidin-3- yl)-2- methylmorpholine | LCMS m/z = 571.2 (M + H); 1HNMR (400 MHz, DMSO-d6) δ 13.11 (s, 1H), 8.57 (s, 2H), 8.36 (s, 1H), 7.73 (dd, J = 12.8 Hz, 1.2 Hz, 1H), 7.48 (d, J = 8.8 Hz, 1H), 7.20 (d, J = 1.6 Hz, 1H), 7.11 (dd, J = 8.8 Hz, 2.0 Hz, 1H), 6.16 (q, J = 6.4 Hz, 1H), 3.99-3.96 (m, 2H), 3.81- 3.77 (m, 3H), 3.52-3.45 (m, 2H), 2.63-2.59 (m, 1H), 2.53- 2.50 (m, 1H), 2.25-2.19 (m, 1H), 1.93 (t, J = 10.4 Hz, 1H), 1.77 (d, J = 6.8 Hz, 3H), 1.33 (s, 3H), 1.08 (d, J = 6.4 Hz, 3H) |
| Example 214. | 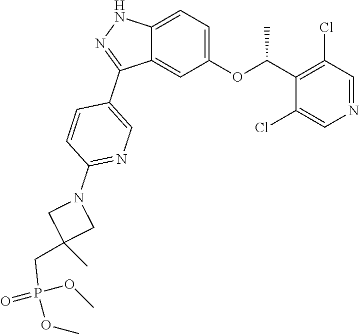 [SMILES: COP(=O)(CC1(C)CN(c2ccc(-c3n[nH]c4ccc(O[C@H](C)c5c(Cl)cncc5Cl)cc34)cn2)C1)OC] | (R)-dimethyl ((1-(5- (5-(1-(3,5- dichloropyridin-4- yl)ethoxy)-1H- indazol-3-yl)pyridin- 2-yl)-3- methylazetidin-3- yl)methyl)phos- phonate | LCMS m/z = 576.2 (M + H); 1HNMR (400 MHz, DMSO-d6) δ 13.00 (s, 1H), 8.60 (s, 2H), 8.50 (d, J = 2.0 Hz, 1H), 7.85 (d, J = 6.4 Hz, 1H), 7.46 (d, J = 9.2 Hz, 1H), 7.16 (s, 1H), 7.09 (dd, J = 9.2 Hz, 2.4 Hz, 1H), 6.54 (d, J = 8.4 Hz, 1H), 6.11 (q, J = 6.8 Hz, 1H), 3.94 (d, J = 8.0 Hz, 2H), 3.73 (d, J = 7.6 Hz, 2H), 3.67 (s, 3H), 3.64 (s, 3H), 2.31-2.26 (d, J = 18.0 Hz, 2 H), 1.76 (d, J = 6.4 Hz, 3H), 1.49 (s, 3H) |
| Example 215. | 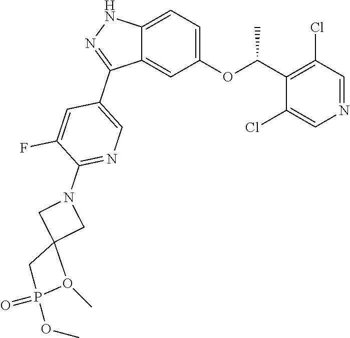 | (R)-dimethyl ((1-(5- (5-(1-(3,5- dichloropyridin-4- yl)ethoxy)-1H- indazol-3-yl)-3- fluoropyridin-2-yl)- 3-methylazetidin-3- yl)methyl)phos- phonate | LCMS m/z = 594.2 (M + H); 1HNMR (400 MHz, DMSO-d6) δ 13.12 (s, 1H), 8.58 (s, 2H), 8.37 (s, 1H), 7.70 (dd, J = 13.2 Hz, 1.6 Hz, 1H), 7.48 (d, J = 9.2 Hz, 1H), 7.20 (d, J = 1.6 Hz, 1H), 7.10 (dd, J = 6.8 Hz, 2.4 Hz, 1H), 6.14 (q, J = 6.4 Hz, 1H), 4.09 (d, J = 7.6 Hz, 2H), 3.83 (d, J = 8.0 Hz, 2H), 3.67 (s, 3H), 3.64 (s, 3H), 2.31-2.27 (d, J = 18.0 Hz, 2H), 1.76 (d, J = 6.4 Hz, 3H), 1.49 (s, 3H) |
| Example 216. | 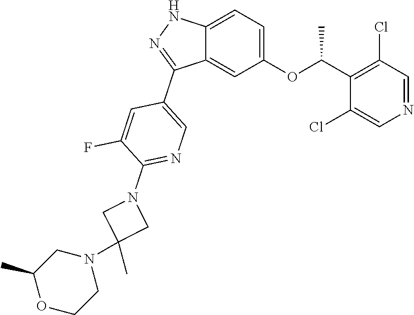 [SMILES: C[C@H]1CN(C2(C)CN(c3ncc(-c4n[nH]c5ccc(O[C@H](C)c6c(Cl)cncc6Cl)cc45)cc3F)C2)CCO1] | (S)-4-(1-(5-(5-((R)- 1-(3,5- dichloropyridin-4- yl)ethoxy)-1H- indazol-3-yl)-3- fluoropyridin-2-yl)- 3-methylazetidin-3- yl)-2- methylmorpholine | LCMS m/z = 571.3 (M + H); 1HNMR (400 MHz, DMSO-d6) δ 13.11 (s, 1H), 8.57 (s, 2H), 8.37-8.37 (d, J = 1.2 Hz, 1H), 7.70 (dd, J = 13.2 Hz, 1.6 Hz, 1H), 7.47 (d, J = 8.8 Hz, 1H), 7.19 (d, J = 2.0 Hz, 1H), 7.10 (dd, J = 9.2 Hz, 2.4 Hz, 1H), 6.14 (q, J = 6.4 Hz, 1H), 3.98 (t, J = 7.2 Hz, 2H), 3.82-3.75 (m, 3H), 3.51-3.44 (m, 2H), 2.63- 2.55 (m, 1H), 2.54-2.51 (m, 1H), 2.22 (td, J = 11.2 Hz, 3.2 Hz, 1H), 1.91 (t, J = 10.0 Hz, 1H), 1.76 (d, J = 6.8 Hz, 3H), 1.34 (s, 3H), 1.07 (d, J = 6.0 Hz, 3H) |
| Example 217. | 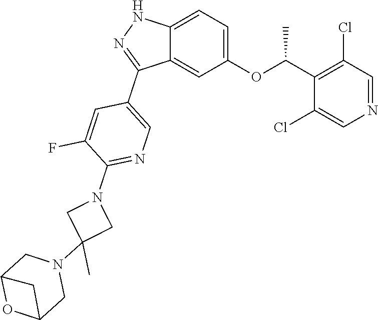 [SMILES: C[C@@H](Oc1ccc2[nH]nc(-c3cnc(N4CC(C)(N5CC6CC(C5)O6)C4)c(F)c3)c2c1)c1c(Cl)cncc1Cl] | 3-(1-(5-(5-((R)-1- (3,5-dichloropyridin- 4-yl)ethoxy)-1H- indazol-3-yl)-3- fluoropyridin-2-yl)- 3-methylazetidin-3- yl)-6-oxa-3-aza- bicyclo[3.1.1]hep- tane | LCMS m/z = 569.3 (M + H); 1HNMR (400 MHz, DMSO-d6) δ 13.12 (s, 1H), 8.57 (s, 2H), 8.38 (s, 1H), 7.72 (dd, J = 13.2 Hz, 1.6 Hz, 1H), 7.47 (d, J = 9.2 Hz, 1H), 7.20 (d, J = 1.6 Hz, 1H), 7.10 (dd, J = 8.8 Hz, 2.0 Hz, 1H), 6.14 (q, J = 6.8 Hz, 1H), 4.49 (d, J = 6.4 Hz, 2H), 4.19 (d, J = 8.4 Hz, 2H), 3.85 (dd, J = 8.4 Hz, 2.0 Hz, 1H), 2.99 (d, J = 10.8 Hz, 2H), 2.88- 2.83 (m, 3H), 2.17 (d, J = 8.0 Hz, 1H), 1.75 (d, J = 6.4 Hz, 3H), 1.51 (s, 3H) |
| Example 218. | 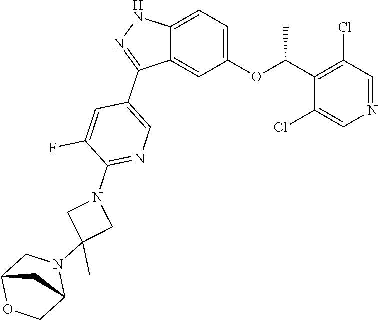 [SMILES: C[C@@H](Oc1ccc2[nH]nc(-c3cnc(N4CC(C)(N5C[C@@H]6C[C@H]5CO6)C4)c(F)c3)c2c1)c1c(Cl)cncc1Cl] | (1S,4S)-5-(1-(5-(5- ((R)-1-(3,5- dichloropyridin-4- yl)ethoxy)-1H- indazol-3-yl)-3- fluoropyridin-2-yl)- 3-methylazetidin-3- yl)-2-oxa-5-aza- bicyclo[2.2.1]hep- tane | LCMS m/z = 569.2 (M + H); 1HNMR (400 MHz, DMSO-d6) δ 13.11 (s, 1H), 8.57 (s, 2H), 8.37 (s, 1H), 7.72 (dd, J = 13.2 Hz, 1.6 Hz, 1H), 7.47 (d, J = 8.8 Hz, 1H), 7.20 (d, J = 2.0 Hz, 1H), 7.11 (dd, J = 8.8 Hz, 2.0 Hz, 1H), 6.15 (q, J = 6.8 Hz, 1H), 4.43 (s, 1H), 4.26 (d, J = 7.6 Hz, 1H), 4.07 (d, J = 8.4 Hz, 1H), 3.93 (dd, J = 8.4 Hz, 1.6 Hz, 1H), 3.87 (dd, J = 8.4 Hz, 2.0 Hz, 1H), 3.74 (s, 1H), 3.59- 3.55 (m, 2H), 2.90 (s, 2H), 1.78-1.73 (m, 4H), 1.64 (d, J = 8.0 Hz, 1H), 1.53 (s, 3H) |
| Example 219. | 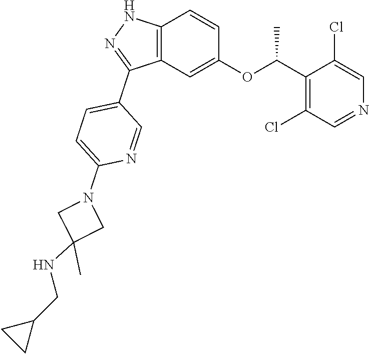 [SMILES: C[C@@H](Oc1ccc2[nH]nc(-c3ccc(N4CC(C)(NCC5CC5)C4)nc3)c2c1)c1c(Cl)cncc1Cl] | (R)-N-(cyclo- propylmethyl)- 1-(5-(5-(1-(3,5- dichloropyridin-4- yl)ethoxy)-1H- indazol-3-yl)pyridin- 2-yl)-3- methylazetidin-3- amine | LCMS m/z = 523.3 (M + H); 1HNMR (400 MHz, DMSO-d6) δ 12.98 (s, 1H), 8.60 (s, 2H), 8.50 (d, J = 2.0 Hz, 1H), 7.84 (dd, J = 8.4 Hz, 2.0 Hz, 1H), 7.46 (d, J = 8.8 Hz, 1H), 7.16 (d, J = 2.0 Hz, 1H), 7.10 (dd, J = 8.8 Hz, 2.0 Hz, 1H), 6.52 (d, J = 8.8 Hz, 1H), 6.13 (q, J = 6.8 Hz, 1H), 3.84 (d, J = 8.0 Hz, 2H), 3.75 (d, J = 8.0 Hz, 2H), 2.41 (d, J = 6.4 Hz, 2H), 1.76 (d, J = 6.4 Hz, 3H), 1.41 (s, 3H), 0.89-0.81 (m, 1H), 0.43- 0.39 (m, 2H), 0.15-0.12 (m, 2H) |
| Example 220. | 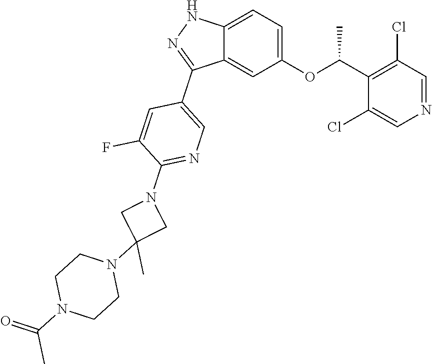 [SMILES: CC(=O)N1CCN(C2(C)CN(c3ncc(-c4n[nH]c5ccc(O[C@H](C)c6c(Cl)cncc6Cl)cc45)cc3F)C2)CC1] | 1-[4-[1-[5-[5-[(1R)- 1-(3,5-dichloro-4- pyridyl)ethoxy]-1H- indazol-3-yl]-3- fluoro-2-pyridyl]-3- methyl-azetidin-3- yl]piperazin-1- yl]ethanone | LCMS m/z = 598 (M + H); 1HNMR (400 MHz, DMSO-d6) δ 13.13 (s, 1H), 8.57 (s, 2H), 8.37 (s, 1H), 7.72 (br d, J = 13.2 Hz, 1H), 7.48 (d, J = 8.9 Hz, 1H), 7.19 (d, J = 2.0 Hz, 1H), 7.10 (dd, J = 2.2, 8.9 Hz, 1H), 6.14 (q, J = 6.6 Hz, 1H), 4.06- 3.96 (m, 3H), 3.83 (br d, J = 7.1 Hz, 2H), 3.44 (br s, 3H), 2.41 (br s, 2H), 2.35 (br s, 2H), 2.00 (s, 3H), 1.76 (d, J = 6.6 Hz, 3H), 1.32 (s, 3H) |
| Example 221. | 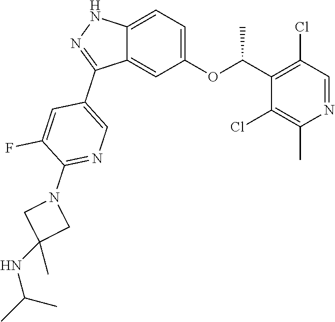 [SMILES: Cc1ncc(Cl)c([C@@H](C)Oc2ccc3[nH]nc(-c4cnc(N5CC(C)(NC(C)C)C5)c(F)c4)c3c2)c1Cl] | 1-[5-[5-[(1R)-1-(3,5- dichloro-2- methyl-4- pyridyl)ethoxy]-1H- indazol-3-yl]-3- fluoro-2-pyridyl]-N- isopropyl-3-methyl- azetidin-3-amine | LCMS m/z= 543.2 (M + H); 1HNMR (400 MHz, DMSO-d6) δ 13.10 (br s, 1H), 8.42 (s, 1H), 8.25 (s, 1H), 7.67 (dd, J = 1.7, 13.2 Hz, 1H), 7.48 (d, J = 8.9 Hz, 1H), 7.10 (dd, J = 2.3, 9.0 Hz, 1H), 7.06 (d, J = 2.0 Hz, 1H), 6.11 (q, J = 6.6 Hz, 1H), 3.97-3.85 (m, 4H), 3.02-2.88 (m, 1H), 2.58 (s, 3H), 1.76 (d, J = 6.7 Hz, 3H), 1.48 (s, 3H), 0.99 (d, J = 6.2 Hz, 6H) |
| Example 222. | 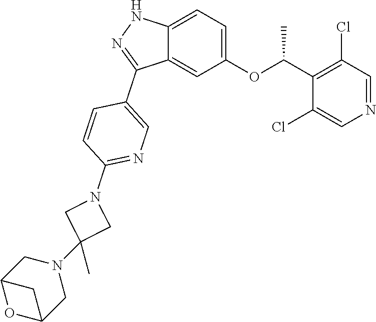 [SMILES: C[C@@H](Oc1ccc2[nH]nc(-c3ccc(N4CC(C)(N5CC6CC(C5)O6)C4)nc3)c2c1)c1c(Cl)cncc1Cl] | 3-(1-(5-(5-((R)-1- (3,5-dichloropyridin- 4-yl)ethoxy)-1H- indazol-3-yl)pyridin- 2-yl)-3- methylazetidin-3- yl)-6-oxa-3-aza- bicyclo[3.1.1]hep- tane | LCMS m/z = 551.3 (M + H); 1HNMR (400 MHz, DMSO-d6) δ 12.99 (s, 1H), 8.59 (s, 2H), 8.52 (d, J = 1.6 Hz, 1H), 7.86 (dd, J = 8.4 Hz, 2.0 Hz, 1H), 7.46 (d, J = 8.8 Hz, 1H), 7.17 (s, 1H), 7.09 (dd, J = 8.8 Hz, 2.0 Hz, 1H), 6.57 (d, J = 8.4 Hz, 1H), 6.11 (q, J = 6.8 Hz, 1H), 4.50 (d, J = 6.0 Hz, 2H), 4.05 (d, J = 8.0 Hz, 2H), 3.73 (d, J = 8.0 Hz, 2H), 3.01 (d, J = 10.8 Hz, 2H), 2.89-2.83 (m, 3H), 2.17 (d, J = 8.0 Hz, 1H), 1.76 (d, J = 6.4 Hz, 3H), 1.50 (s, 3H) |
| Example 223. | 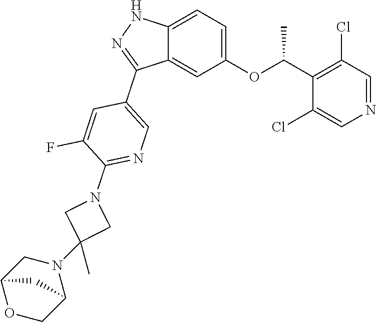 [SMILES: C[C@@H](Oc1ccc2[nH]nc(-c3cnc(N4CC(C)(N5C[C@H]6C[C@@H]5CO6)C4)c(F)c3)c2c1)c1c(Cl)cncc1Cl] | (1R,4R)-5-(1-(5-(5- ((R)-1-(3,5- dichloropyridin-4- yl)ethoxy)-1H- indazol-3-yl)-3- fluoropyridin-2-yl)- 3-methylazetidin-3- yl)-2-oxa-5-aza- bicyclo[2.2.1]hep- tane | LCMS m/z = 569.3 (M + H); 1HNMR (400 MHz, DMSO-d6) δ 13.10 (s, 1H), 8.27 (s, 2H), 8.36 (t, J = 1.6 Hz, 1H), 7.69 (dd, J = 13.2 Hz, 2.0 Hz, 1H), 7.47 (d, J = 9.2 Hz, 1H), 7.20 (d, J = 2.0 Hz, 1H), 7.10 (dd, J = 9.2 Hz, 2.0 Hz, 1H), 6.14 (q, J = 6.8 Hz, 1H), 4.43 (d, J = 1.6 Hz, 1H), 4.27 (d, J = 8.0 Hz, 1H), 4.07 (d, J = 8.8 Hz, 1H), 3.92 (d, J = 8.4 Hz, 1H), 3.86 (dd, J = 8.4 Hz, 1.6 Hz, 1H), 3.75 (s, 1H), 3.59-3.56 (m, 2H), 2.90 (s, 2H), 1.77-1.73 (m, 4H), 1.65 (d, J = 7.2 Hz, 1H), 1.52 (s, 3H) |
| Example 224. | 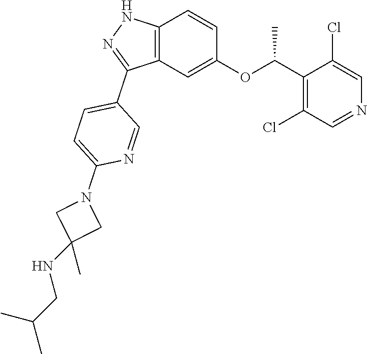 [SMILES: CC(C)CNC1(C)CN(c2ccc(-c3n[nH]c4ccc(O[C@H](C)c5c(Cl)cncc5Cl)cc34)cn2)C1] | (R)-1-(5-(5-(1-(3,5- dichloropyridin-4- yl)ethoxy)-1H- indazol-3-yl)pyridin- 2-yl)-N-isobutyl-3- methylazetidin-3- amine | LCMS m/z = 525.3 (M + H); 1HNMR (400 MHz, DMSO-d6) δ 12.98 (s, 1H), 8.60 (s, 2H), 8.51 (s, 1H), 7.83 (d, J = 8.0 Hz, 1H), 7.46 (d, J = 8.8 Hz, 1H), 7.17 (s, 1H), 7.09 (d, J = 8.8 Hz, 1H), 6.52 (d, J = 8.4 Hz, 1H), 6.12 (q, J = 6.4 Hz, 1H), 3.84 (d, J = 7.6 Hz, 2H), 3.75 (d, J = 8 Hz, 2H), 2.35 (d, J = 5.2 Hz, 2H), 1.76 (d, J = 6.8 Hz, 3H), 1.63-1.60 (m, 1H), 1.43 (s, 3H), 0.91 (d, J = 6.4 Hz, 6H) |
| Example 225. | 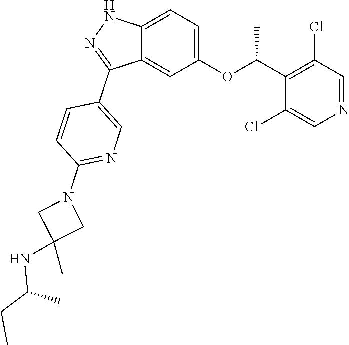 [SMILES: CC[C@@H](C)NC1(C)CN(c2ccc(-c3n[nH]c4ccc(O[C@H](C)c5c(Cl)cncc5Cl)cc34)cn2)C1] | 1-[5-[5-[(1R)-1-(3,5- dichloro-4- pyridyl)ethoxy]-1H- indazol-3-yl]-2- pyridyl]-3-methyl- N-[(1R)-1-meth- ylpropyl]azetidin- 3-amine | LCMS m/z = 525 (M + H); 1HNMR (400 MHz, DMSO-d6) δ 10.20 (br s, 1H), 10.10-10.00 (m, 1H), 8.60 (s, 2H), 8.38 (d, J = 1.7 Hz, 1H), 8.26 (br d, J = 8.9 Hz, 1H), 7.52 (d, J = 9.0 Hz, 1H), 7.20 (d, J = 2.1 Hz, 1H), 7.14-7.11 (m, 1H), 7.02 (br d, J = 9.0 Hz, 1H), 6.14 (q, J = 6.7 Hz, 1H), 4.69-4.57 (m, 2H), 4.28 (br d, J = 9.7 Hz, 2H), 3.30 (br s, 1H), 1.79-1.73 (m, 7H), 1.61-1.50 (m, 1H), 1.24 (d, J = 6.5 Hz, 3H), 0.96 (t, J = 7.5 Hz, 3H) |
| Example 226. | 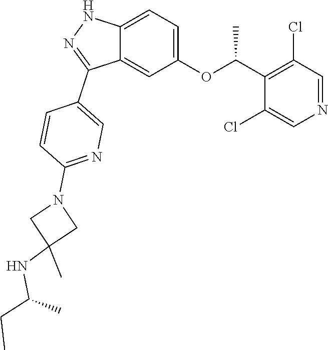 | (R)-1-(1-(5-(5-(1- (3,5-dichloropyridin- 4-yl)ethoxy)-1H- indazol-3-yl)pyridin- 2-yl)-3- methylazetidin-3- yl)-4-methyl-1,4- azaphosphinane 4- oxide | LCMS m/z = 585.3 (M + H); 1HNMR (400 MHz, DMSO-d6) δ 13.00 (s, 1H), 8.60 (s, 2H), 8.51 (d, J = 1.6 Hz, 1H), 7.86 (d, J = 10.4 Hz, 1H), 7.46 (d, J = 9.2 Hz, 1H), 7.16 (s, 1H), 7.09 (d, J = 11.6 Hz, 1H), 6.54 (d, J = 8.4 Hz, 1H), 6.12 (q, J = 6.8 Hz , 1H), 3.77-3.73 (m, 4H), 2.80-2.76 (m, 2H), 2.53-2.47 (m, 2H), 1.93-1.79 (m, 4H), 1.76 (d, J = 6.8 Hz, 3H), 1.42 (d, J = 13.2 Hz, 3H), 1.34 (s, 3H) |
| Example 227. | 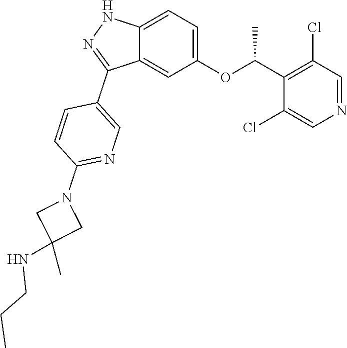 [SMILES: CCCNC1(C)CN(c2ccc(-c3n[nH]c4ccc(O[C@H](C)c5c(Cl)cncc5Cl)cc34)cn2)C1] | 1-[5-[5-[(1R)-1-(3,5- dichloro-4- pyridyl)ethoxy]-1H- indazol-3-yl]-2- pyridyl]-3-methyl- N-propyl-azetidin-3- amine | LCMS m/z = 511 (M + H); 1HNMR (400 MHz, DMSO-d6) δ 9.89 (br s, 2H), 8.59 (s, 2H), 8.44-8.41 (m, 1H), 8.20 (br d, J = 8.3 Hz, 1H), 7.51 (d, J = 9.0 Hz, 1H), 7.20 (d, J = 2.1 Hz, 1H), 7.12 (dd, J = 2.3, 9.0 Hz, 1H), 6.92 (br d, J = 9.2 Hz, 1H), 6.14 (q, J = 6.8 Hz, 1H), 4.49 (br d, J = 9.5 Hz, 2H), 4.18 (br d, J = 9.8 Hz, 2H), 2.97-2.89 (m, 2H), 1.77 (d, J = 6.6 Hz, 3H), 1.74-1.67 (m, 5H), 0.98 (t, J = 7.4 Hz, 3H) |
| Example 228. | 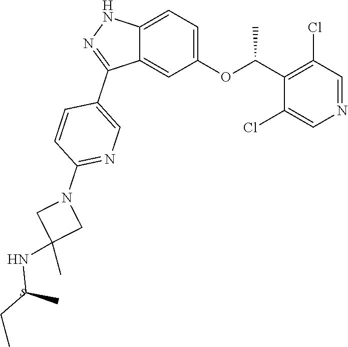 | 1-[5-[5-[(1R)-1-(3,5- dichloro-4- pyridyl)ethoxy]-1H- indazol-3-yl]-2- pyridyl]-3-methyl- N-[(1S)-1-meth- ylpropyl]azetidin- 3-amine | LCMS m/z = 527.2 (M + H); 1HNMR (400 MHz, DMSO-d6) δ 13.65-12.93 (m, 1H), 9.97 (br s, 1H), 9.88 (br s, 1H), 8.60 (s, 2H), 8.42 (d, J = 1.7 Hz, 1H), 8.21 (br d, J = 8.7 Hz, 1H), 7.51 (d, J = 9.0 Hz, 1H), 7.20 (d, J = 2.1 Hz, 1H), 7.12 (dd, J = 2.3, 9.0 Hz, 1H), 6.96 (br d, J = 8.8 Hz, 1H), 6.14 (q, J = 6.6 Hz, 1H), 4.60-4.48 (m, 2H), 4.24 (br d, J = 9.3 Hz, 2H), 3.31 (br s, 1H), 1.78-1.75 (m, 6H), 1.60-1.50 (m, 1H), 1.23 (d, J = 6.4 Hz, 3H), 0.96 (t, J = 7.5 Hz, 3H) |
| Example 229. | 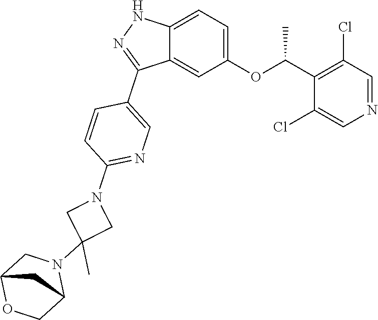 [SMILES: C[C@@H](Oc1ccc2[nH]nc(-c3ccc(N4CC(C)(N5C[C@@H]6C[C@H]5CO6)C4)nc3)c2c1)c1c(Cl)cncc1Cl] | (1S,4S)-5-(1-(5-(5- ((R)-1-(3,5- dichloropyridin-4- yl)ethoxy)-1H- indazol-3-yl)pyridin- 2-yl)-3- methylazetidin-3- yl)-2-oxa-5-aza- bicyclo[2.2.1]hep- tane | LCMS m/z = 551.3 (M + H); 1HNMR (400 MHz, DMSO-d6) δ 12.97 (s, 1H), 8.59 (s, 2H), 8.51 (d, J = 1.6 Hz, 1H), 7.85 (dd, J = 8.4 Hz, 2.4 Hz, 1H), 7.44 (d, J = 9.2 Hz, 1H), 7.17 (s, 1H), 7.10 (dd, J = 8.8 Hz, 1.6 Hz, 1H), 6.55 (d, J = 8.4 Hz, 1H), 6.13 (q, J = 6.8 Hz, 1H), 4.43 (s, 1H), 4.12 (d, J = 8.4 Hz, 1H), 3.95 (d, J = 8.8 Hz, 1H), 3.81 (d, J = 8.0 Hz, 1H), 3.76-3.73 (m, 2H), 3.61-3.55 (m, 2H), 2.91-2.83 (m, 2H), 1.77-1.75 (m, 4H), 1.67 (d, J = 9.6 Hz, 1H), 1.51 (s, 3H) |
| Example 230. | 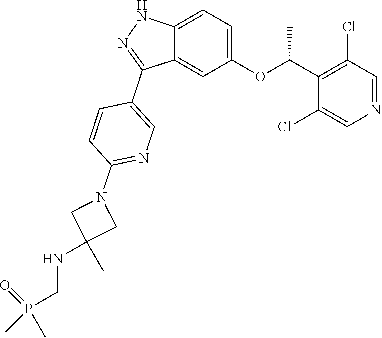 | 1-[5-[5-[(1R)-1-(3,5- dichloro-4- pyridyl)ethoxy]-1H- indazol-3-yl]-2- pyridyl]-N-(di- methylphosphoryl- methyl)-3-methyl- azetidin-3-amine | LCMS m/z = 589 (M + H); 1HNMR (400 MHz, DMSO-d6) δ 13.01 (s, 1H), 8.58 (s, 2H), 8.52 (d, J = 2.1 Hz, 1H), 7.85 (dd, J = 2.3, 8.6 Hz, 1H), 7.46 (d, J = 8.9 Hz, 1H), 7.17 (s, 1H), 7.09 (dd, J = 2.2, 9.0 Hz, 1H), 6.51 (d, J = 8.7 Hz, 1H), 6.09 (q, J = 6.7 Hz, 1H), 3.88 (d, J = 8.1 Hz, 2H), 3.74 (d, J = 8.1 Hz, 2H), 2.84 (br d, J = 7.7 Hz, 2H), 1.75 (d, J = 6.6 Hz, 3H), 1.43 (s, 3H), 1.42 (s, 3H), 1.39 (s, 3H) |
| Example 231. | 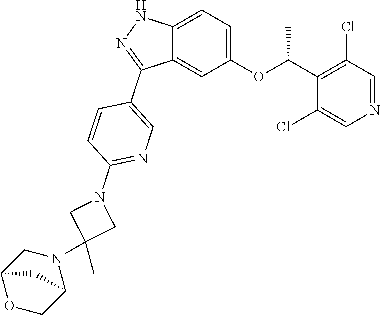 [SMILES: C[C@@H](Oc1ccc2[nH]nc(-c3ccc(N4CC(C)(N5C[C@H]6C[C@@H]5CO6)C4)nc3)c2c1)c1c(Cl)cncc1Cl] | (1R,4R)-5-(1-(5-(5- ((R)-1-(3,5- dichloropyridin-4- yl)ethoxy)-1H- indazol-3-yl)pyridin- 2-yl)-3- methylazetidin-3- yl)-2-oxa-5-aza- bicyclo[2.2.1]hep- tane | LCMS m/z = 551.3 (M + H); 1HNMR (400 MHz, DMSO-d6) δ 12.98 (s, 1H), 8.59 (s, 2H), 8.50 (d, J = 1.6 Hz, 1H), 7.84 (dd, J = 8.4 Hz, 2.0 Hz, 1H), 7.45 (d, J = 9.2 Hz, 1H), 7.17 (s, 1H), 7.09 (d, J = 9.2 Hz, 2.4 Hz, 1H), 6.54 (d, J = 8.4 Hz, 1H), 6.11 (q, J = 6.8 Hz, 1H), 4.43 (s, 1H), 4.11 (d, J = 8.0 Hz, 1H), 3.93 (d, J = 8.4 Hz, 1H), 3.79 (d, J = 8.4 Hz, 1H), 3.75-3.71 (m, 2H), 3.61-3.55 (m, 2H), 2.92-2.84 (m, 2H), 1.77-1.75 (m, 4H), 1.67-1.64 (m, 1H), 1.51 (s, 3H) |
| Example 232. | 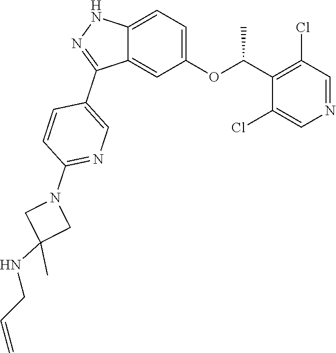 [SMILES: C=CCNC1(C)CN(c2ccc(-c3n[nH]c4ccc(O[C@H](C)c5c(Cl)cncc5Cl)cc34)cn2)C1] | (R)-N-ally1-1-(5-(5- (1-(3,5- dichloropyridin-4- yl)ethoxy)-1H- indazol-3-yl)pyridin- 2-yl)-3- methylazetidin-3- amine | LCMS m/z = 509.2 (M + H); 1HNMR (400 MHz, DMSO-d6) δ 12.97 (s, 1H), 8.59 (s, 2H), 8.50 (d, J = 1.6 Hz, 1H), 7.83 (dd, J = 8.8 Hz, 2.4 Hz, 1H), 7.45 (d, J = 9.2 Hz, 1H), 7.16 (d, J = 1.6 Hz, 1H), 7.09 (dd, J = 9.2 Hz, 2.4 Hz, 1H), 6.52 (d, J = 8.4 Hz, 1H), 6.10 (q, J = 6.8 Hz, 1H), 5.96-5.85 (m, 1H), 5.24-5.19 (m, 1H), 5.06 (dd, J = 10.0 Hz, 1.6 Hz, 1H), 3.85 (d, J = 8.0 Hz, 2H), 3.75 (d, J = 8.0 Hz, 2H), 3.21 (d, J = 6.0 Hz, 2H), 1.76 (d, J = 6.4 Hz, 3H), 1.44 (s, 3H) |
| Example 233. | 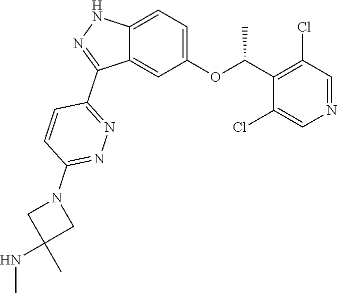 [SMILES: CNC1(C)CN(c2ccc(-c3n[nH]c4ccc(O[C@H](C)c5c(Cl)cncc5Cl)cc34)nn2)C1] | (R)-1-(5-(5-(1-(3,5- dichloropyridin-4- yl)ethoxy)-1H- indazol-3-yl)pyridin- 2-yl)-N,3- dimethylazetidin-3- amine | LCMS m/z = 483.2 (M + H); 1HNMR (400 MHz, DMSO-d6) δ 12.97 (s, 1H), 8.59 (s, 2H), 8.50 (d, J = 2.0 Hz, 1H), 7.83 (dd, J = 8.4 Hz, 2.4 Hz, 1H), 7.45 (d, J = 9.2 Hz, 1H), 7.17 (d, J = 2.0 Hz, 1H), 7.09 (dd, J = 8.8 Hz, 2.0 Hz, 1H), 6.53 (d, J = 8.8 Hz, 1H), 6.11 (q, J = 6.8 Hz, 1H), 3.83 (d, J = 7.6 Hz, 2H), 3.73 (d, J = 8.0 Hz, 2H), 2.25 (s, 3H), 1.76 (d, J = 6.8 Hz, 3H), 1.40 (s, 3H) |
| Example 234. | 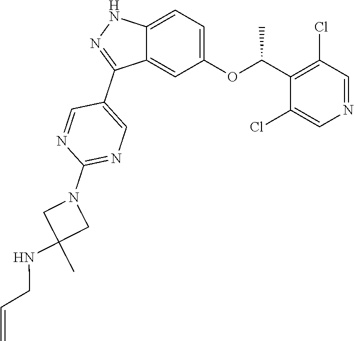 [SMILES: C=CCNC1(C)CN(c2ncc(-c3n[nH]c4ccc(O[C@H](C)c5c(Cl)cncc5Cl)cc34)cn2)C1] | (R)-N-ally1-1-(5-(5- (1-(3,5- dichloropyridin-4- yl)ethoxy)-1H- indazol-3- yl)pyrimidin-2-yl)- 3-methylazetidin-3- amine | LCMS m/z = 510.2 (M + H); 1HNMR (400 MHz, DMSO-d6) δ 13.12 (s, 1H), 8.75 (s, 2H), 8.57 (s, 2H), 7.47 (d, J = 9.2 Hz, 1H), 7.25 (d, J = 1.2 Hz, 1H), 7.10 (dd, J = 2.4 Hz, 9.2 Hz, 1H), 6.15 (q, J = 6.4 Hz, 1H), 5.86-5.60 (m, 1H), 5.21 (dd, J = 17.6 Hz, 1.6 Hz, 1H), 5.04 (d, J = 10.4 Hz, 1H), 3.96 (d, J = 8.8 Hz, 2H), 3.82 (d, J = 8.8 Hz, 2H), 3.20 (d, J = 5.6 Hz, 2H), 1.75 (d, J = 6.8 Hz, 3H), 1.43 (s, 3H) |
| Example 235. | 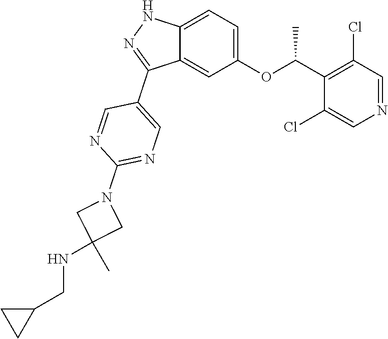 [SMILES: C[C@@H](Oc1ccc2[nH]nc(-c3cnc(N4CC(C)(NCC5CC5)C4)nc3)c2c1)c1c(Cl)cncc1Cl] | (R)-N-(cyclo- propylmethyl)- 1-(5-(5-(1-(3,5- dichloropyridin-4- yl)ethoxy)-1H- indazol-3- yl)pyrimidin-2-yl)- 3-methylazetidin-3- amine | LCMS m/z = 524.3 (M + H); 1HNMR (400 MHz, DMSO-d6) δ 13.11 (brs, 1H), 8.76 (s, 2H), 8.57 (s, 2H), 7.47 (d, J = 8.8 Hz, 1H), 7.25 (d, J = 2.0 Hz, 1H), 7.10 (dd, J = 8.8 Hz, 2.4 Hz, 1H), 6.15 (q, J = 6.8 Hz, 1H), 4.00-3.98 (m, 2H), 3.85- 3.83 (m, 2H), 2.49-2.42 (m, 2H), 1.76 (d, J = 6.4 Hz, 3H), 1.43 (s, 3H), 0.92-0.84 (m, 1H), 0.45-0.42 (m, 2H), 0.18-0.16 (m, 2H) |
| Example 236. | 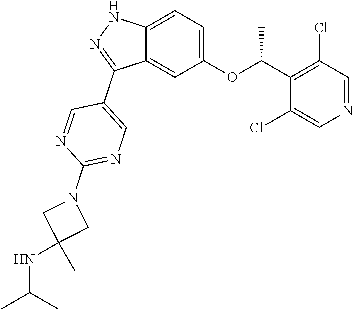 [SMILES: CC(C)NC1(C)CN(c2ncc(-c3n[nH]c4ccc(O[C@H](C)c5c(Cl)cncc5Cl)cc34)cn2)C1] | (R)-1-(5-(5-(1-(3,5- dichloropyridin-4- yl)ethoxy)-1H- indazol-3- yl)pyrimidin-2-yl)- N-isopropyl-3- methylazetidin-3- amine | LCMS m/z= 512.3 (M + H); 1HNMR (400 MHz, DMSO-d6) δ 13.12 (s, 1H), 8.75 (s, 2H), 8.57 (s, 2H), 7.48 (d, J = 8.8 Hz, 1H), 7.25 (s, 1H), 7.11 (dd, J = 9.2 Hz, 1.2 Hz, 1H), 6.16 (q, J = 6.4 Hz, 1H), 3.88 (s, 4H), 3.01-2.87 (m, 1H)), 1.76 (d, J = 6.0 Hz, 3H), 1.45 (s, 3H), 1.00 (d, J = 6.0 Hz, 6H) |
| Example 237. | 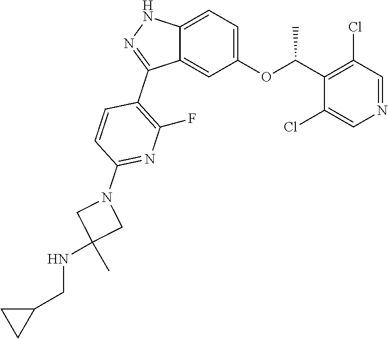 [SMILES: C[C@@H](Oc1ccc2[nH]nc(-c3ccc(N4CC(C)(NCC5CC5)C4)nc3F)c2c1)c1c(Cl)cncc1Cl] | (R)-N-(cyclo- propylmethyl)- 1-(5-(5-(1-(3,5- dichloropyridin-4- yl)ethoxy)-1H- indazol-3-yl)-6- fluoropyridin-2-yl)- 3-methylazetidin-3- amine | LCMS m/z = 541.3 (M + H); 1HNMR (400 MHz, DMSO-d6) δ 13.10 (s, 1H), 8.58 (s, 2H), 7.83 (t, J = 8.4 Hz, 1H), 7.47 (d, J = 9.2 Hz, 1H), 7.11 (dd, J = 8.8 Hz, 2.4 Hz, 1H), 6.91 (s, 1H), 6.39 (dd, J = 8.0 Hz, 1.6 Hz, 1H), 6.03 (q, J = 6.4 Hz, 1H), 3.87 (d, J = 8.0 Hz, 2H), 3.76 (dd, J = 8.0 Hz, 2.0 Hz, 2H), 2.42 (d, J = 6.4 Hz, 2H), 1.74 (d, J = 6.4 Hz, 3H), 1.41 (s, 3H), 0.90-0.83 (m, 1H), 0.44-0.39 (m, 2H), 0.16-0.12 (m, 2H) |
| Example 238. | 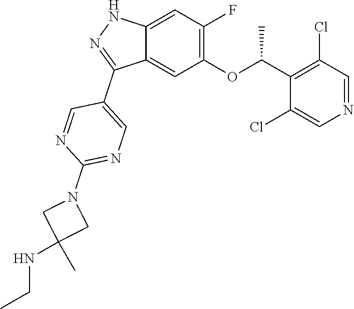 [SMILES: CCNC1(C)CN(c2ncc(-c3n[nH]c4cc(F)c(O[C@H](C)c5c(Cl)cncc5Cl)cc34)cn2)C1] | (R)-1-(5-(5-(1-(3,5- dichloropyridin-4- yl)ethoxy)-1H- indazol-3- yl)pyrimidin-2-yl)- N-ethyl-3- methylazetidin-3- amine | LCMS m/z = 498.2 (M + H); 1HNMR (400 MHz, DMSO-d6) δ 13.13 (s, 1H), 8.75 (s, 2H), 8.57 (s, 2H), 7.47 (d, J = 9.2 Hz, 1H), 7.25 (d, J = 1.6 Hz, 1H), 7.10 (dd, J = 8.8 Hz, 2.0 Hz, 1H), 6.16 (q, J = 6.8 Hz, 1H), 3.95 (d, J = 8.4Hz, 2H), 3.82 (d, J = 8.8 Hz, 2H), 2.58- 2.55 (m, 2H), 1.75 (d, J = 6.8 Hz, 3H), 1.41 (s, 3H), 1.04 (t, J = 7.2 Hz, 3H) |
| Example 239. | 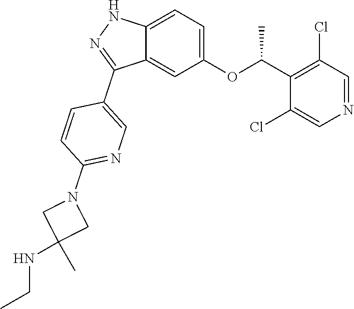 [SMILES: CCNC1(C)CN(c2ccc(-c3n[nH]c4ccc(O[C@H](C)c5c(Cl)cncc5Cl)cc34)cn2)C1] | 1-[5-[5-[(1R)-1-(3,5- dichloro-4- pyridyl)ethoxy]-1H- indazol-3-yl]-2- pyridyl]-N-ethyl-3- methyl-azetidin-3- amine | LCMS m/z = 497.1 (M + H); 1HNMR (400 MHz, DMSO-d6) δ 13.35 (br s, 1H), 10.03 (br s, 2H), 8.60 (s, 2H), 8.40 (br s, 1H), 8.26 (br d, J = 8.1 Hz, 1H), 7.52 (br d, J = 9.3 Hz, 1H), 7.20 (br s, 1H), 7.13 (br d, J = 9.3 Hz, 1H), 6.98 (br d, J = 8.8 Hz, 1H), 6.14 (br d, J = 7.0 Hz, 1H), 4.53 (br d, J = 9.7 Hz, 2H), 4.21 (br d, J = 9.3 Hz, 2H), 3.03 (br s, 2H), 1.77 (br d, J = 6.0 Hz, 3H), 1.70 (s, 3H), 1.28 (br t, J = 6.7 Hz, 3H) |
| Example 240. | 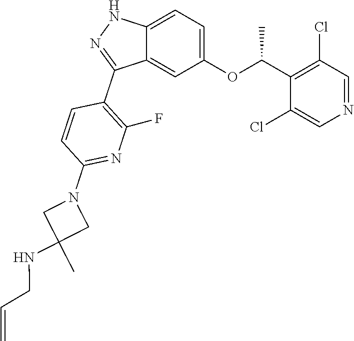 [SMILES: C=CCNC1(C)CN(c2ccc(-c3n[nH]c4ccc(O[C@H](C)c5c(Cl)cncc5Cl)cc34)c(F)n2)C1] | (R)-N-ally1-1-(5-(5- (1-(3,5- dichloropyridin-4- yl)ethoxy)-1H- indazol-3-yl)-6- fluoropyridin-2-yl)- 3-methylazetidin-3- amine | LCMS m/z = 527.2 (M + H); 1HNMR (400 MHz, DMSO-d6) δ ) 8.58 (s, 2H), 7.85-7.80 (m, 1H), 7.47 (d, J = 8.8 Hz, 1H), 7.11 (dd, J = 8.8 Hz, 1.6 Hz, 1H), 6.91 (t, J = 1.6 Hz, 1H), 6.39 (dd, J = 8.0 Hz, 2.4 Hz, 1H), 6.03 (q, J = 6.4 Hz, 1H), 5.94-5.85 (m, 1H), 5.23 (dd, J = 17.2 Hz, 1.6 Hz, 1H), 5.05 (dd, J = 10.0 Hz, 2.4 Hz, 1H), 3.87 (dd, J = 8.0 Hz, 2.4 Hz, 2H), 3.76 (dd, J = 8.0 Hz, 1.2 Hz, 2H), 3.19 (d, J = 5.6 Hz, 2H), 1.74 (d, J = 6.8 Hz, 3H), 1.43 (s, 3H) |
| Example 241. | 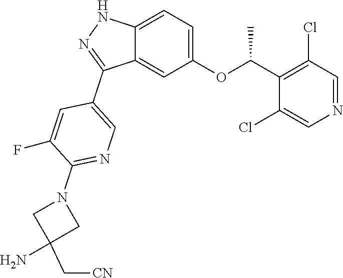 [SMILES: C[C@@H](Oc1ccc2[nH]nc(-c3cnc(N4CC(N)(CC#N)C4)c(F)c3)c2c1)c1c(Cl)cncc1Cl] | (R)-2-(3-amino-1- (5-(5-(1-(3,5- dichloropyridin-4- yl)ethoxy)-1H- indazol-3-yl)-3- fluoropyridin-2- yl)azetidin-3- yl)acetonitrile | LCMS m/z = 512.2 (M + H); 1HNMR (400 MHz, DMSO-d6) δ 13.10 (s, 1H), 8.54 (s, 2H), 8.35 (s, 1H), 7.69 (d, J = 13.2 Hz, 1H), 7.44 (d, J = 8.8 Hz, 1H), 7.17 (s, 1H), 7.06 (d, J = 9.2 Hz, 2.0 Hz, 1H), 6.11 (q, J = 6.4 Hz, 1H), 4.02 (d, J = 7.6 Hz, 2H), 3.92 (d, J = 8.0 Hz, 2H), 2.99 (s, 2H), 1.72 (d, J = 6.8 Hz, 3H) |
| Example 242. | 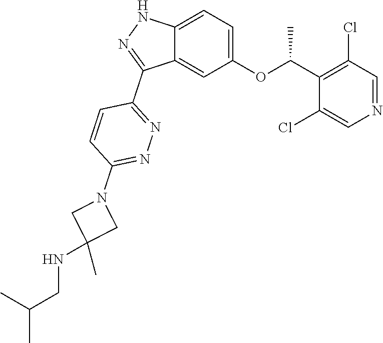 [SMILES: CC(C)CNC1(C)CN(c2ccc(-c3n[nH]c4ccc(O[C@H](C)c5c(Cl)cncc5Cl)cc34)nn2)C1] | (R)-1-(6-(5-(1-(3,5- dichloropyridin-4- yl)ethoxy)-1H- indazol-3- yl)pyridazin-3-yl)- N-isobutyl-3- methylazetidin-3- amine | LCMS m/z = 526.4 (M + H); 1HNMR (400 MHz, DMSO-d6) δ 8.56 (s, 2H), 7.97-7.94 (m, 2H), 7.49 (d, J = 9.2 Hz, 1H), 7.10 (dd, J = 9.2 Hz, 2.4 Hz, 1H), 6.88 (d, J = 9.2 Hz, 1H), 6.10 (q, J = 6.8 Hz, 1H), 3.94 (d, J = 8.0 Hz, 2H), 3.85 (d, J = 8.0 Hz, 2H), 2.35 (d, J = 6.4 Hz, 2H), 1.77 (d, J = 6.8 Hz, 3H), 1.63-1.56 (m, 1H), 1.44 (s, 3H), 0.91 (d, J = 6.4 Hz, 6H) |
| Example 243. | 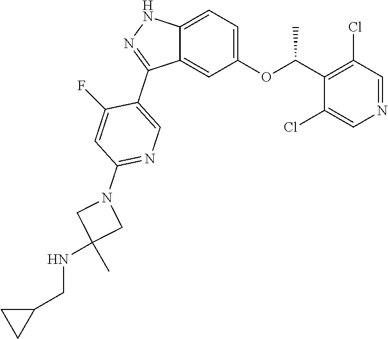 [SMILES: C[C@@H](Oc1ccc2[nH]nc(-c3cnc(N4CC(C)(NCC5CC5)C4)cc3F)c2c1)c1c(Cl)cncc1Cl] | (R)-N-(cyclo- propylmethyl)- 1-(5-(5-(1-(3,5- dichloropyridin-4- yl)ethoxy)-1H- indazol-3-yl)-4- fluoropyridin-2-yl)- 3-methylazetidin-3- amine | LCMS m/z = 541.3 (M + H); 1HNMR (400 MHz, DMSO-d6) δ 13.14 (s, 1H), 8.59 (s, 2H), 8.29 (d, J = 10.8 Hz, 1H), 7.47 (d, J = 8.8 Hz, 1H), 7.10 (dd, J = 8.8 Hz, 2.0 Hz, 1H), 6.79 (s, 1H), 6.38 (d, J = 12.8 Hz, 1H), 5.98 (q, J = 6.4 Hz, 1H), 3.85 (d, J = 8.0 Hz, 2H), 3.75 (dd, J = 8.0 Hz, 1.2 Hz, 2H), 2.40 (d, J = 6.4 Hz, 2H), 1.73 (d, J = 6.4 Hz, 3H), 1.41 (s, 3H), 0.89-0.83 (m, 1H), 0.44-0.39 (m, 2H), 0.16-0.12 (m, 2H) |
| Example 244. | 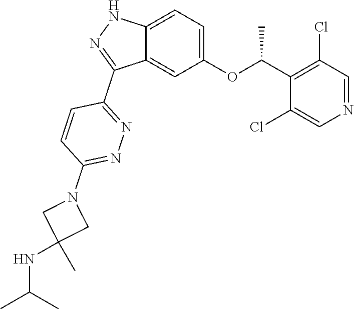 [SMILES: CC(C)NC1(C)CN(c2ccc(-c3n[nH]c4ccc(O[C@H](C)c5c(Cl)cncc5Cl)cc34)nn2)C1] | (R)-1-(6-(5-(1-(3,5- dichloropyridin-4- yl)ethoxy)-1H- indazol-3- yl)pyridazin-3-yl)- N-isopropyl-3- methylazetidin-3- amine | LCMS m/z = 512.3 (M + H); 1HNMR (400 MHz, DMSO-d6) δ 13.19 (s, 1H), 8.56 (s, 2H), 7.97-7.94 (m, 2H), 7.48 (d, J = 8.8 Hz, 1H), 7.10 (dd, J = 9.2 Hz, 2.4 Hz, 1H), 6.89 (d, J = 8.8 Hz, 1H), 6.14 (q, J = 6.4 Hz, 1H), 3.93 (d, J = 8.0 Hz, 2H), 3.88 (d, J = 7.6 Hz, 2H), 2.99- 2.93 (m, 1H), 1.77 (d, J = 6.4 Hz, 3H), 1.49 (s, 3H), 1.01 (d, J = 6.4 Hz, 6H) |
| Example 245. | 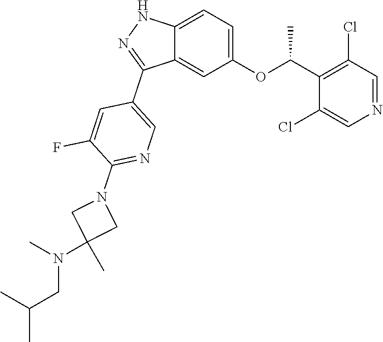 [SMILES: CC(C)CN(C)C1(C)CN(c2ncc(-c3n[nH]c4ccc(O[C@H](C)c5c(Cl)cncc5Cl)cc34)cc2F)C1] | (R)-1-(5-(5-(1-(3,5- dichloropyridin-4- yl)ethoxy)-1H- indazol-3-yl)-3- fluoropyridin-2-yl)- N-isobutyl-N,3- dimethylazetidin-3- amine | LCMS m/z = 557.3 (M + H); 1HNMR (400 MHz, DMSO-d6) δ 13.12 (s, 1H), 8.57 (s, 2H), 8.37 (s, 1H), 7.71 (dd, J = 13.2 Hz, 1.6 Hz, 1H), 7.48 (d, J = 9.2 Hz, 1H), 7.20 (d, J = 1.6 Hz, 1H), 7.10 (dd, J = 9.2 Hz, 2.4 Hz, 1H), 6.14 (q, J = 6.8 Hz, 1H), 3.87-3.80 (m, 4H), 2.07 (s, 3H), 1.99 (d, J = 7.2 Hz, 2H), 1.76 (d, J = 6.4 Hz, 3H), 1.70- 1.64 (m, 1H), 1.32 (s, 3H), 0.87 (d, J = 6.0 Hz, 6H) |
| Example 246. | 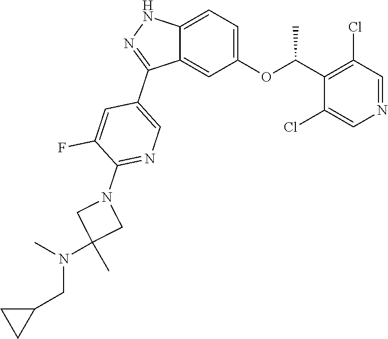 [SMILES: C[C@@H](Oc1ccc2[nH]nc(-c3cnc(N4CC(C)(N(C)CC5CC5)C4)c(F)c3)c2c1)c1c(Cl)cncc1Cl] | (R)-N- (cyclopropyl- methyl)- 1-(5-(5-(1-(3,5- dichloropyridin-4- yl)ethoxy)-1H- indazol-3-yl)-3- fluoropyridin-2-yl)- N,3- dimethylazetidin-3- amine | LCMS m/z = 555.3 (M + H); 1HNMR (400 MHz, DMSO-d6) δ 13.12 (s, 1H), 8.57 (s, 2H), 8.37 (s, 1H), 7.72 (d, J = 13.6 Hz, 1H), 7.49 (d, J = 8.8 Hz, 1H), 7.20 (d, J = 2.0 Hz, 1H), 7.11 (dd, J = 8.8 Hz, 2.0 Hz, 1H), 6.17 (q, J = 7.2 Hz, 1H), 3.92 (d, J = 7.6 Hz, 2H), 3.82 (d, J = 7.2 Hz, 2H), 2.18 (s, 3H), 2.13 (d, J = 6.4 Hz, 2H), 1.77 (d, J = 6.4 Hz, 3H), 1.30 (s, 3H), 0.85-0.78 (m, 1H), 0.48-0.46 (m, 2H), 0.13-0.08 (m, 2H) |
| Example 247. | 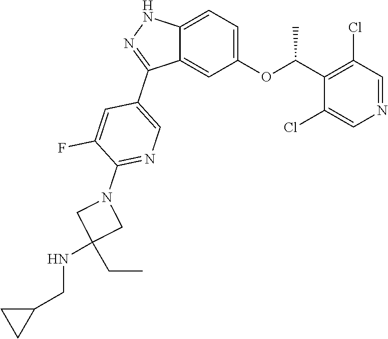 [SMILES: CCC1(NCC2CC2)CN(c2ncc(-c3n[nH]c4ccc(O[C@H](C)c5c(Cl)cncc5Cl)cc34)cc2F)C1] | N- (cyclopropyl- methyl)- 1-[5-[5-[(1R)-1- (3,5-dichloro-4- pyridyl)ethoxy]-1H- indazol-3-yl]-3- fluoro-2-pyridyl]-3- ethyl-azetidin-3- amine | LCMS m/z = 555.2 (M + H); 1HNMR (400 MHz, DMSO-d6) δ 13.20 (br s, 1H), 9.76 (br s, 2H), 8.57 (s, 2H), 8.41 (s, 1H), 7.80 (dd, J = 1.7, 13.0 Hz, 1H), 7.49 (d, J = 9.0 Hz, 1H), 7.20 (d, J = 2.1 Hz, 1H), 7.11 (dd, J = 2.1, 9.0 Hz, 1H), 6.14 (q, J = 6.7 Hz, 1H), 4.41 (br d, J = 9.2 Hz, 2H), 4.15 (br d, J = 8.9 Hz, 2H), 2.91-2.83 (m, 2H), 1.99 (q, J = 7.3 Hz, 2H), 1.76 (d, J = 6.6 Hz, 3H), 1.14-1.04 (m, 4H), 0.64-0.56 (m, 2H), 0.42 (q, J = 4.7 Hz, 2H) |
| Example 248. | 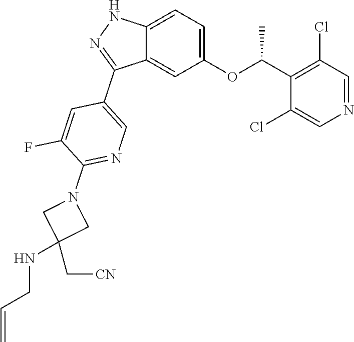 [SMILES: C=CCNC1(CC#N)CN(c2ncc(-c3n[nH]c4ccc(O[C@H](C)c5c(Cl)cncc5Cl)cc34)cc2F)C1] | 2-[3-(allylamino)-1- [5-[5-[(1R)-1-(3,5- dichloro-4- pyridyl)ethoxy]-1H- indazol-3-yl]-3- fluoro-2- pyridyl]azetidin-3- yl]acetonitrile | LCMS m/z = 552 (M + H); 1HNMR (400 MHz, DMSO-d6) δ 13.11 (s, 1H), 8.57 (s, 2H), 8.39 (s, 1H), 7.73 (dd, J = 1.7, 13.1 Hz, 1H), 7.47 (d, J = 8.9 Hz, 1H), 7.25-7.17 (m, 1H), 7.10 (dd, J = 2.1, 9.0 Hz, 1H), 6.14 (q, J = 6.6 Hz, 1H), 5.98- 5.84 (m, 1H), 5.26 (dd, J = 1.7, 17.1 Hz, 1H), 5.07 (dd, J = 1.6, 10.3 Hz, 1H), 4.12-4.04 (m, 2H), 4.03-3.96 (m, 2H), 3.27- 3.19 (m, 2H), 3.14 (s, 2H), 2.77 (br t, J = 6.5 Hz, 1H), 1.76 (d, J = 6.6 Hz, 3H) |
| Example 249. | 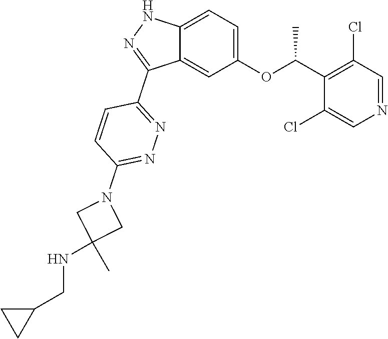 [SMILES: C[C@@H](Oc1ccc2[nH]nc(-c3ccc(N4CC(C)(NCC5CC5)C4)nn3)c2c1)c1c(Cl)cncc1Cl] | (R)-N- (cyclopropyl- methyl)- 1-(6-(5-(1-(3,5- dichloropyridin-4- yl)ethoxy)-1H- indazol-3- yl)pyridazin-3-yl)-3- methylazetidin-3- amine | LCMS m/z = 524.3 (M + H); 1HNMR (400 MHz, DMSO-d6) δ 13.18 (s, 1H), 8.56 (s, 2H), 7.97-7.94 (m, 2H), 7.47 (d, J = 9.2 Hz, 1H), 7.09 (d, J = 8.8 Hz, 1H), 6.87 (d, J = 9.2 Hz, 1H), 6.10 (q, J = 6.4 Hz, 1H), 3.95 (d, J = 8.0 Hz, 2H), 3.84 (d, J = 8.0 Hz, 2H), 2.41 (d, J = 6.0 Hz, 2H), 1.77 (d, J = 6.6 Hz, 3H), 1.44 (s, 3H), 0.88-0.87 (m, 1H), 0.41 (d, J = 7.2 Hz, 2H), 0.14 (d, J = 4.0 Hz, 2H) |
| Example 250. | 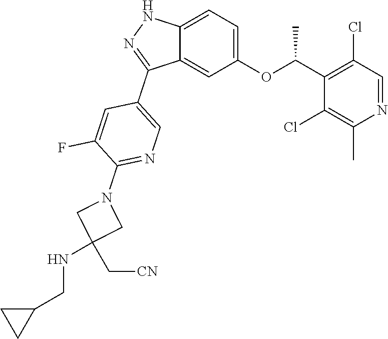 [SMILES: Cc1ncc(Cl)c([C@@H](C)Oc2ccc3[nH]nc(-c4cnc(N5CC(CC#N)(NCC6CC6)C5)c(F)c4)c3c2)c1Cl] | 2-[3- (cyclopropylmethyl- amino)-1-[5-[5- [(1R)-1-(3,5-di- chloro-2-methyl-4- pyridyl)ethoxy]-1H- indazol-3-yl]-3- fluoro-2- pyridyl]azetidin-3- yl]acetonitrile | LCMS m/z = 580 (M + H); 1HNMR (400 MHz, DMSO-d6) δ 13.19 (br s, 1H), 10.30 (br s, 2H), 8.42 (s, 1H), 8.34 (s, 1H), 7.81 (dd, J = 1.5, 13.0 Hz, 1H), 7.49 (d, J = 8.9 Hz, 1H), 7.13- 7.07 (m, 2H), 6.12 (q, J = 6.7 Hz, 1H), 4.57 (br d, J = 9.9 Hz, 2H), 4.33-4.25 (m, 2H), 3.58 (s, 2H), 2.97 (br s, 2H), 2.58 (s, 3H), 1.76 (d, J = 6.6 Hz, 3H), 1.17-1.09 (m, 1H), 0.66-0.56 (m, 2H), 0.50-0.40 (m, 2H) |
| Example 251. | 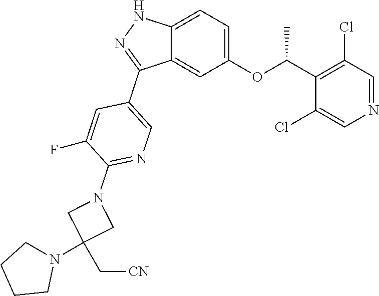 [SMILES: C[C@@H](Oc1ccc2[nH]nc(-c3cnc(N4CC(CC#N)(N5CCCC5)C4)c(F)c3)c2c1)c1c(Cl)cncc1Cl] | (R)-2-(1-(5-(5-(1- (3,5-dichloropyridin- 4-yl)ethoxy)-1H- indazol-3-yl)-3- fluoropyridin-2-yl)- 3-(pyrrolidin-1- yl)azetidin-3- yl)acetonitrile | LCMS m/z = 566.2 (M + H); 1HNMR (400 MHz, DMSO-d6) δ 13.14 (s, 1H), 8.57 (s, 2H), 8.39 (s, 1H), 7.73 (dd, J = 7.6 Hz, 1.6 Hz, 1H), 7.48 (d, J = 9.2 Hz, 1H), 7.20 (d, J = 1.6 Hz, 1H), 7.10 (dd, J = 8.8 Hz, 2.0 Hz, 1H), 6.14 (q, J = 6.8 Hz, 1H), 4.26 (d, J = 8.8 Hz, 2H), 4.00 (d, J = 7.6 Hz, 2H), 3.19 (s, 2H), 2.72-2.65 (m, 4H), 1.77-1.75 (m, 7H) |
| Example 252. | 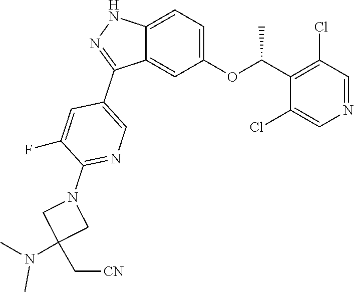 [SMILES: C[C@@H](Oc1ccc2[nH]nc(-c3cnc(N4CC(CC#N)(N(C)C)C4)c(F)c3)c2c1)c1c(Cl)cncc1Cl] | (R)-2-(1-(5-(5-(1- (3,5-dichloropyridin- 4-yl)ethoxy)-1H- indazol-3-yl)-3- fluoropyridin-2-yl)- 3-(dimethyl- amino)azeti- din-3-yl)acetonitrile | LCMS m/z = 540.3 (M + H); 1HNMR (400 MHz, DMSO-d6) δ 13.14 (s, 1H), 8.57 (s, 2H), 8.39 (s, 1H), 7.74 (d, J = 13.2 Hz, 1H), 7.48 (d, J = 8.8 Hz, 1H), 7.20 (s, 1H), 7.10 (d, J = 8.8 Hz, 1H), 6.15 (q, J = 6.8 Hz, 1H), 4.09-4.03 (m, 4H), 3.08 (s, 2H), 2.22 (s, 6H), 1.76 (d, J = 6.4 Hz, 3H) |
| Example 253. | 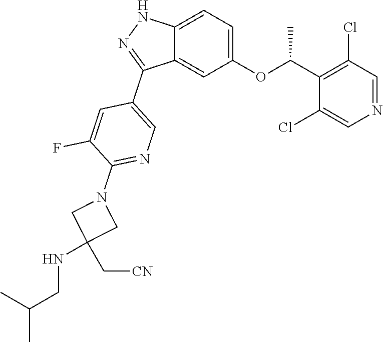 [SMILES: CC(C)CNC1(CC#N)CN(c2ncc(-c3n[nH]c4ccc(O[C@H](C)c5c(Cl)cncc5Cl)cc34)cc2F)C1] | (R)-2-(1-(5-(5-(1- (3,5- Dichloropyridin-4- yl)ethoxy)-1H- indazol-3-yl)-3- fluoropyridin-2-yl)- 3-(iso- butylamino)azeti- din-3-yl)acetonitrile dihydrochloride | LCMS m/z= 568.2 (M + H); 1HNMR (400 MHz, DMSO-d6) δ 13.19 (br s, 1H), 10.01 (br s, 2H), 8.57 (s, 2H), 8.44 (s, 1H), 7.83 (d, J = 13.0 Hz, 1H), 7.49 (d, J = 8.9 Hz, 1H), 7.21 (s, 1H), 7.11 (d, J = 9.1 Hz, 1H), 6.15 (q, J = 6.6 Hz, 1H), 4.56 (br d, J = 10.0 Hz, 2H), 4.28 (br d, J = 9.9 Hz, 2H), 3.59 (s, 2H), 2.89 (br s, 2H), 2.10-1.99 (m, 1H), 1.77 (d, J = 6.6 Hz, 3H), 1.00 (d, J = 6.6 Hz, 6H) |
| Example 254. | 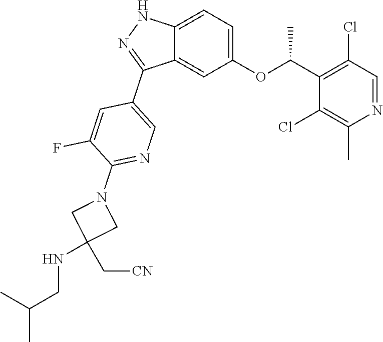 [SMILES: Cc1ncc(Cl)c([C@@H](C)Oc2ccc3[nH]nc(-c4cnc(N5CC(CC#N)(NCC(C)C)C5)c(F)c4)c3c2)c1Cl] | (R)-2-(1-(5-(5-(1- (3,5-Dichloro-2- methylpyridin-4- yl)ethoxy)-1H- indazol-3-yl)-3- fluoropyridin-2-yl)- 3-(iso- butylamino)azeti- din-3-yl)acetonitrile | LCMS m/z = 582.2 (M + H); 1HNMR (400 MHz, DMSO-d6) δ 13.11 (s, 1H), 8.42 (s, 1H), 8.27 (s, 1H), 7.71 (d, J = 13.1 Hz, 1H), 7.48 (d, J = 8.9 Hz, 1H), 7.12-7.05 (m, 2H), 6.11 (q, J = 6.5 Hz, 1H), 4.07-3.98 (m, 5H), 3.11 (s, 2H), 2.59 (s, 3H), 2.34 (br s, 2H), 1.76 (d, J = 6.5 Hz, 3H), 1.61 (quind, J = 6.6, 13.2 Hz, 1H), 0.92 (d, J = 6.5 Hz, 6H) |
| Example 255. | 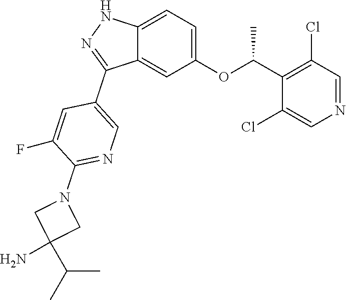 [SMILES: CC(C)C1(N)CN(c2ncc(-c3n[nH]c4ccc(O[C@H](C)c5c(Cl)cncc5Cl)cc34)cc2F)C1] | (R)-1-(5-(5-(1-(3,5- dichloropyridin-4- yl)ethoxy)-1H- indazol-3-yl)-3- fluoropyridin-2-yl)- 3-isopropylazetidin- 3-amine | LCMS m/z = 515.3 (M + H); 1HNMR (400 MHz, DMSO-d6) δ 13.11 (s, 1H), 8.57 (s, 2H), 8.36 (d, J = 2.8 Hz, 1H), 7.68 (dd, J = 13.2 Hz, 2.0 Hz, 1H), 7.47 (d, J = 8.8 Hz, 1H), 7.19 (d, J = 2.0 Hz, 1H), 7.10 (dd, J = 9.2 Hz, 2.0 Hz, 1H), 6.14 (q, J = 6.4 Hz, 1H), 4.02 (dd, J = 8.4 Hz, 1.6 Hz, 2H), 3.81 (d, J = 7.6 Hz, 2H), 1.98-1.91 (m, 1H), 1.76 (d, J = 6.8 Hz, 3H), 0.92 (d, J = 7.2 Hz, 6H) |
| Example 256. | 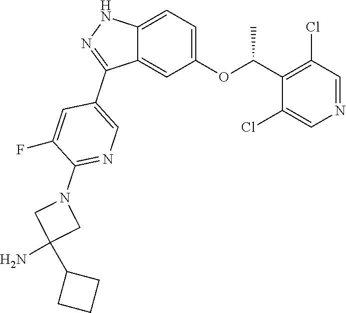 [SMILES: C[C@@H](Oc1ccc2[nH]nc(-c3cnc(N4CC(N)(C5CCC5)C4)c(F)c3)c2c1)c1c(Cl)cncc1Cl] | (R)-3-cyclobutyl-1- (5-(5-(1-(3,5- dichloropyridin-4- yl)ethoxy)-1H- indazol-3-yl)-3- fluoropyridin-2- yl)azetidin-3-amine | LCMS m/z = 527.3 (M + H); 1HNMR (400 MHz, DMSO-d6) δ 13.12 (s, 1H), 8.57 (s, 2H), 8.36 (t, J = 1.2 Hz, 1H)), 7.69 (dd, J = 13.2 Hz, 2.0 Hz, 1H), 7.47 (d, J = 9.2 Hz, 1H), 7.20 (d, J = 2.0 Hz, 1H), 7.10 (dd, J = 8.8 Hz, 2.4 Hz, 1H), 6.15 (q, J = 6.8 Hz, 1H), 4.04-4.02 (m, 2H), 3.85-3.82 (m, 2H), 2.68- 2.66 (m, 1H), 1.97-1.82 (m, 6H), 1.76 (d, J = 6.4 Hz, 3H) |
| Example 257. | 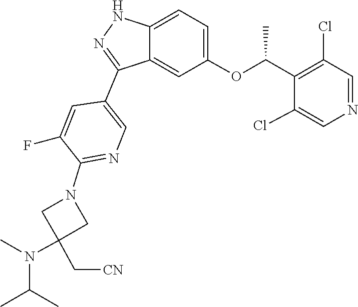 [SMILES: CC(C)N(C)C1(CC#N)CN(c2ncc(-c3n[nH]c4ccc(O[C@H](C)c5c(Cl)cncc5Cl)cc34)cc2F)C1] | (R)-2-(1-(5-(5-(1- (3,5-dichloropyridin- 4-yl)ethoxy)-1H- indazol-3-yl)-3- fluoropyridin- 2-yl)-3- (isopropyl(methyl)a- mino)azetidin-3- yl)acetonitrile | LCMS m/z = 568.3 (M + H); 1HNMR (400 MHz, DMSO-d6) δ 13.14 (s, 1H), 8.57 (s, 2H), 8.39 (s, 1H), 7.76 (dd, J = 13.2 Hz, 1.6 Hz, 1H), 7.49 (d, J = 9.2 Hz, 1H), 7.21 (d, J = 1.2 Hz, 1H), 7.12 (dd, J = 8.8 Hz, 2.0 Hz, 1H), 6.17 (q, J = 6.4 Hz, 1H), 4.08-4.03 (m, 4H), 3.11 (s, 2H), 3.00-2.94 (m, 1H), 2.17 (s, 3H), 1.77 (d, J = 6.8 Hz, 3H), 1.02 (d, J = 6.4 Hz, 6H) |
| Example 258. | 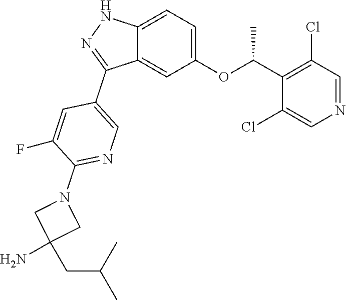 [SMILES: CC(C)CC1(N)CN(c2ncc(-c3n[nH]c4ccc(O[C@H](C)c5c(Cl)cncc5Cl)cc34)cc2F)C1] | (R)-1-(5-(5-(1-(3,5- dichloropyridin-4- yl)ethoxy)-1H- indazol-3-yl)-3- fluoropyridin-2-yl)- 3-isobutylazetidin-3- amine | LCMS m/z = 529.3 (M + H); 1HNMR (400 MHz, DMSO-d6) δ 13.11 (s, 1H), 8.58 (s, 2H), 8.36 (s, 1H), 7.68 (dd, J = 12.8 Hz, 1.2 Hz, 1H), 7.47 (d, J = 9.2 Hz, 1H), 7.19 (s, 1H), 7.10 (dd, J = 8.8 Hz, 2.4 Hz, 1H), 6.14 (q, J = 6.8 Hz, 1H), 4.02 (d, J = 8.4 Hz, 2H), 3.89 (d, J = 8.0 Hz, 2H), 1.93-1.87 (m, 1H), 1.76 (d, J = 6.4 Hz, 3H), 1.65 (d, J = 6.8 Hz, 2H), 0.93 (d, J = 6.8 Hz, 6H). |
| Example 259. | 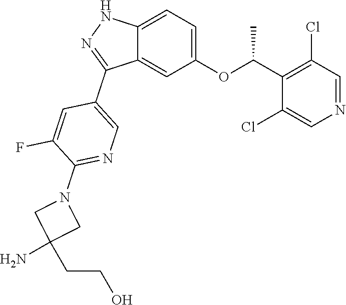 [SMILES: C[C@@H](Oc1ccc2[nH]nc(-c3cnc(N4CC(N)(CCO)C4)c(F)c3)c2c1)c1c(Cl)cncc1Cl] | (R)-2-(3-amino-1- (5-(5-(1-(3,5- dichloropyridin-4- yl)ethoxy)-1H- indazol-3-yl)-3- fluoropyridin-2- yl)azetidin-3- yl)ethanol | LCMS m/z = 517.2 (M + H); 1HNMR (400 MHz, DMSO-d6) δ 13.11 (s, 1H), 8.57 (s, 2H), 8.36 (s, 1H), 7.68 (dd, J = 13.2 Hz, 1.6 Hz, 1H), 7.47 (d, J = 9.2 Hz, 1H), 7.19 (d, J = 2.4 Hz, 1H), 7.10 (dd, J = 8.8 Hz, 2.0 Hz, 1H), 6.14 (q, J = 6.8 Hz, 1H), 4.05 (dd, J = 8.4 Hz, 2.0 Hz, 2H), 3.85 (d, J = 7.6 Hz, 2H), 3.65 (t, J = 6.8 Hz, 2H), 1.90 (t, J = 6.0 Hz, 2H), 1.76 (d, J = 6.4 Hz, 3H) |
| Example 260. | 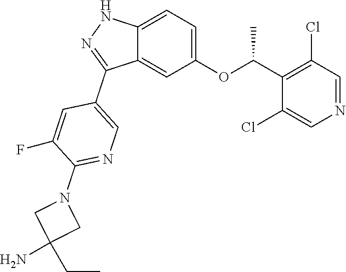 [SMILES: CCC1(N)CN(c2ncc(-c3n[nH]c4ccc(O[C@H](C)c5c(Cl)cncc5Cl)cc34)cc2F)C1] | 1-[5-[5-[(1R)-1-(3,5- dichloro-4- pyridyl)ethoxy]-1H- indazol-3-yl]-3- fluoro-2-pyridyl]-3- ethyl-azetidin-3- amine | LCMS m/z = 501.1 (M+H); 1HNMR (400 MHz, DMSO-d6) δ 8.68 (br s, 3H), 8.57 (s, 2H), 8.41 (t, J = 1.5 Hz, 1H), 7.81 (d, J = 1.7 Hz, 1H), 7.77 (d, J = 1.7 Hz, 1H), 7.49 (d, J = 9.0 Hz, 1H), 7.20 (d, J = 2.2 Hz, 1H), 7.11 (dd, J = 2.3, 9.0 Hz, 1H), 6.14 (q, J = 6.6 Hz, 1H), 4.25 (d, J = 8.4 Hz, 2H), 4.17 (d, J = 8.3 Hz, 2H), 3.44 (q, J = 7.0 Hz, 1H), 2.00-1.90 (m, 2H), 1.76 (d, J = 6.6 Hz, 3H), 1.05 (dt, J = 1.6, 7.2 Hz, 4H) |
| Example 261. | 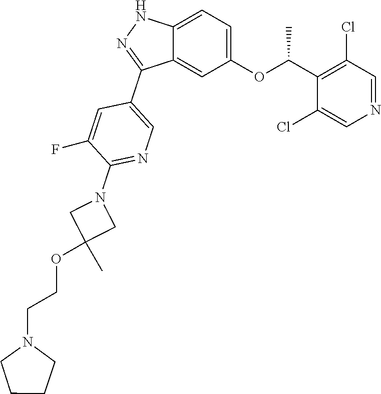 [SMILES: C[C@@H](Oc1ccc2[nH]nc(-c3cnc(N4CC(C)(OCCN5CCCC5)C4)c(F)c3)c2c1)c1c(Cl)cncc1Cl] | (R)-5-(1-(3,5- dichloropyridin-4- yl)ethoxy)-3-(5- fluoro-6-(3-methyl- 3-(2-(pyrrolidin-1- yl)ethoxy)azetidin- 1-yl)pyridin-3-yl)- 1H-indazole | LCMS m/z = 585.3 (M + H); 1HNMR (400 MHz, DMSO-d6) δ 13.17 (s, 1H), 8.57 (s, 2H), 8.38 (s, 1H), 7.73 (dd, J = 13.2 Hz, 1.2 Hz, 1H), 7.48 (d, J = 9.2 Hz, 1H), 7.19 (d, J = 1.6 Hz, 1H), 7.10 (dd, J = 9.2 Hz, 2.4 Hz, 1H), 6.14 (q, J = 6.8 Hz, 1H), 4.15 (d, J = 8.4 Hz, 2H), 4.00 (d, J = 8.0 Hz, 2H), 3.69 (t, J = 5.2 Hz, 2H), 3.28-3.01 (m, 6H), 1.92-1.85 (m, 4H), 1.76 (d, J = 6.8 Hz, 3H), 1.56 (s, 3H) |
| Example 262. | 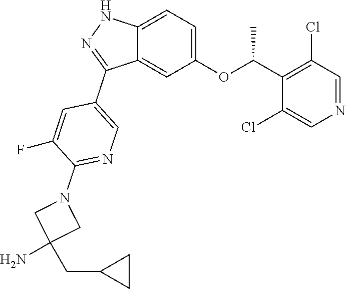 [SMILES: C[C@@H](Oc1ccc2[nH]nc(-c3cnc(N4CC(N)(CC5CC5)C4)c(F)c3)c2c1)c1c(Cl)cncc1Cl] | (R)-3-(cyclo- propylmethyl)- 1-(5-(5-(1-(3,5- dichloropyridin-4- yl)ethoxy)-1H- indazol-3-yl)-3- fluoropyridin-2- yl)azetidin-3-amine | LCMS m/z = 527.3 (M + H); 1HNMR (400 MHz, DMSO-d6) δ 13.10 (brs, 1H), 8.57 (s, 2H), 8.36 (d, J = 1.6 Hz, 1H), 7.67 (dd, J = 13.2 Hz, 2.0 Hz, 1H), 7.47 (d, J = 9.2Hz, 1H), 7.19 (s, 1H), 7.10 (dd, J = 8.8 Hz, 1.6 Hz, 1H), 6.14 (q, J = 6.4 Hz, 1H), 4.07 (d, J = 8.0 Hz, 2H), 3.87 (d, J = 8.0 Hz, 2H), 1.76 (d, J = 6.4 Hz, 3H), 1.63 (d, J = 6.4 Hz, 2H), 0.88-0.84 (m, 1H), 0.47-0.45 (m, 2H), 0.16-0.14 (m, 2H) |
| Example 263. | 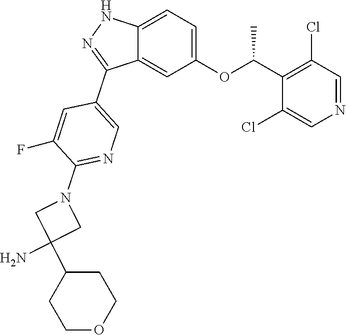 [SMILES: C[C@@H](Oc1ccc2[nH]nc(-c3cnc(N4CC(N)(C5CCOCC5)C4)c(F)c3)c2c1)c1c(Cl)cncc1Cl] | (R)-1-(5-(5-(1-(3,5- dichloropyridin-4- yl)ethoxy)-1H- indazol-3-yl)-3- fluoropyridin-2-yl)- 3-(tetrahydro-2H- pyran-4-yl)azetidin- 3-amine | LCMS m/z = 557.3 (M + H); 1HNMR (400 MHz, DMSO-d6) δ 13.14 (s, 1H), 8.57 (s, 2H), 8.39 (s, 1H), 7.74 (d, J = 13.6 Hz, 1H), 7.48 (d, J = 9.2 Hz, 1H), 7.19 (s, 1H), 7.11 (dd, J = 8.8 Hz, 2.0 Hz, 1H), 6.14 (q, J = 6.6 Hz, 1H), 4.17 (d, J = 8.8 Hz, 2H), 4.02-3.92 (m, 4H), 3.33- 3.26 (m, 2H), 1.98-1.92 (m, 1H), 1.76 (d, J = 6.6 Hz, 3H), 1.66-1.61 (m, 2H), 1.49-1.39 (m, 2H) |
| Example 264. | 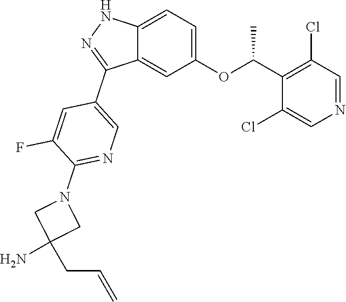 [SMILES: C=CCC1(N)CN(c2ncc(-c3n[nH]c4ccc(O[C@H](C)c5c(Cl)cncc5Cl)cc34)cc2F)C1] | (R)-3-ally1-1-(5-(5- (1-(3,5- dichloropyridin-4- yl)ethoxy)-1H- indazol-3-yl)-3- fluoropyridin-2- yl)azetidin-3-amine | LCMS m/z = 513.3 (M + H); 1HNMR (400 MHz, DMSO-d6) δ 13.12 (s, 1H), 8.58 (s, 2H), 8.37 (s, 1H), 7.70 (dd, J = 13.2 Hz, 1.6 Hz, 1H), 7.47 (d, J = 8.8 Hz, 1H), 7.20 (d, J = 2.0 Hz, 1H), 7.10 (dd, J = 8.8 Hz, 2.0 Hz, 1H), 6.15 (q, J= 6.8 Hz, 1H), 6.01-5.94 (m, 1H), 5.22-5.14 (m, 2H), 4.00 (d, J = 6.8 Hz, 2H), 3.86 (d, J = 8.0 Hz, 2H), 2.47 (d, J = 7.6 Hz, 2H), 1.76 (d, J = 6.8 Hz, 3H) |
| Example 265. | 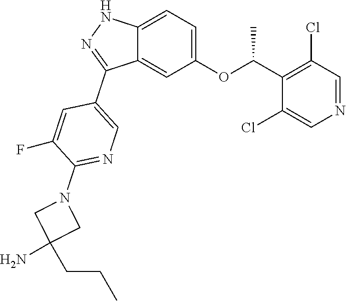 [SMILES: CCCC1(N)CN(c2ncc(-c3n[nH]c4ccc(O[C@H](C)c5c(Cl)cncc5Cl)cc34)cc2F)C1] | (R)-1-(5-(5-(1-(3,5- dichloropyridin-4- yl)ethoxy)-1H- indazol-3-yl)-3- fluoropyridin-2-yl)- 3-propylazetidin-3- amine | LCMS m/z = 515.3 (M +H); 1HNMR (400 MHz, DMSO-d6) δ 13.11 (s, 1H), 8.57 (s, 2H), 8.36 (s, 1H), 7.68 (dd, J = 13.2 Hz, 1.2 Hz, 1H), 7.47 (d, J = 9.2 Hz, 1H), 7.19 (s, 1H), 7.10 (dd, J = 8.8 Hz, 2.0 Hz, 1H), 6.14 (q, J = 6.4 Hz, 1H), 3.96 (d, J = 6.8 Hz, 2H), 3.84 (d, J = 7.6 Hz, 2H), 1.76 (d, J = 6.8 Hz, 3H), 1.69-1.65 (m, 2H), 1.47-1.38 (m, 2H), 0.95 (t, J = 7.2 Hz, 3H) |
| Example 266. | 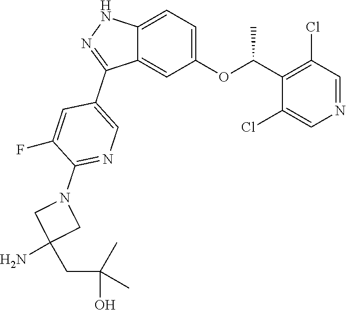 [SMILES: C[C@@H](Oc1ccc2[nH]nc(-c3cnc(N4CC(N)(CC(C)(C)O)C4)c(F)c3)c2c1)c1c(Cl)cncc1Cl] | (R)-1-(3-amino-1- (5-(5-(1-(3,5- dichloropyridin-4- yl)ethoxy)-1H- indazol-3-yl)-3- fluoropyridin-2- yl)azetidin-3-yl)-2- methylpropan-2-ol | LCMS m/z = 545.3 (M + H); 1HNMR (400 MHz, DMSO-d6) δ 13.14 (s, 1H), 8.57 (s, 2H), 8.37 (s, 1H), 7.69 (d, J = 13.2 Hz, 1H), 7.47 (d, J = 9.2 Hz, 1H), 7.19 (s, 1H), 7.05 (dd, J = 9.2 Hz, 1.6 Hz, 1H), 6.14 (q, J = 6.0 Hz, 1H), 4.43 (brs, 1H), 4.16 (d, J = 8.0 Hz, 2H), 3.97 (d, J = 8.0 Hz, 2H), 1.92 (s, 2H), 1.76 (d, J = 6.8 Hz, 3H), 1.18 (s, 6H) |
| Example 267. | 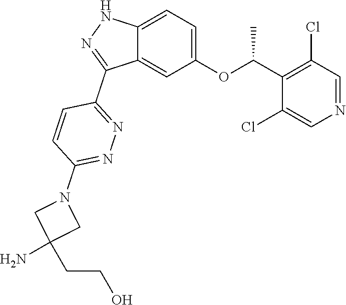 [SMILES: C[C@@H](Oc1ccc2[nH]nc(-c3ccc(N4CC(N)(CCO)C4)nn3)c2c1)c1c(Cl)cncc1Cl] | (R)-2-(3-amino-1- (6-(5-(1-(3,5- dichloropyridin-4- yl)ethoxy)-1H- indazol-3- yl)pyridazin-3- yl)azetidin-3- yl)ethanol | LCMS m/z = 500.2 (M + H); 1HNMR (400 MHz, DMSO-d6) δ 13.18 (s, 1H), 8.56 (s, 2H), 8.01-7.92 (m, 2H), 7.47 (d, J = 9.2 Hz, 1H), 7.09 (dd, J = 8.8 Hz, 2.4 Hz, 1H), 6.88 (d, J = 8.8 Hz, 1H), 6.08 (q, J = 6.8 Hz, 1H), 4.07 (d, J = 8.4 Hz, 2H), 3.84 (d, J = 7.6 Hz, 2H), 3.67 (t, J = 6.4 Hz, 2H), 1.93 (t, J = 6.4 Hz, 2H), 1.77 (d, J = 6.4 Hz, 3H) |
| Example 268. | 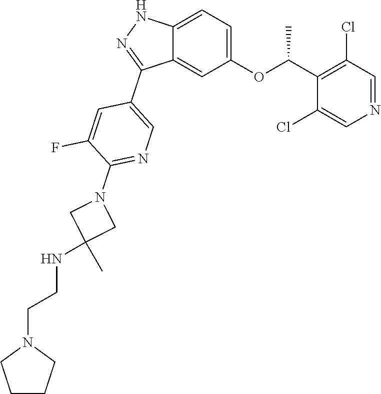 [SMILES: C[C@@H](Oc1ccc2[nH]nc(-c3cnc(N4CC(C)(NCCN5CCCC5)C4)c(F)c3)c2c1)c1c(Cl)cncc1Cl] | (R)-1-(5-(5-(1-(3,5- dichloropyridin-4- yl)ethoxy)-1H- indazol-3-yl)-3- fluoropyridin-2-yl)- 3-methyl-N-(2- (pyrrolidin-1- yl)ethyl)azetidin-3- amine | LCMS m/z = 584.4 (M + H); 1HNMR (400 MHz, DMSO-d6) δ 13.12 (s, 1H), 8.58 (s, 2H), 8.36 (t, J = 1.6 Hz, 1H), 7.69 (dd, J = 13.2 Hz, 1.6 Hz, 1H), 7.47 (d, J = 9.2 Hz, 1H), 7.19 (d, J = 2.0 Hz, 1H), 7.10 (dd, J = 9.2 Hz, 2.4 Hz, 1H), 6.14 (q, J = 6.4 Hz, 1H), 3.97 (d, J = 8.4 Hz, 2H), 3.87 (d, J = 8.4 Hz, 2H), 2.68-2.62 (m, 2H), 2.54- 2.53 (m, 2H), 2.49-2.43 (m, 4H), 1.76 (d, J = 6.4 Hz, 3H), 1.69-1.67 (m, 4H), 1.43 (s, 3H) |
| Example 269. | 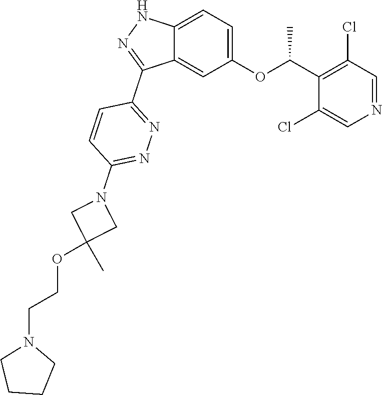 [SMILES: C[C@@H](Oc1ccc2[nH]nc(-c3ccc(N4CC(C)(OCCN5CCCC5)C4)nn3)c2c1)c1c(Cl)cncc1Cl] | (R)-5-(1-(3,5- dichloropyridin-4- yl)ethoxy)-3-(6-(3- methyl-3-(2- (pyrrolidin-1- yl)ethoxy)azetidin- 1-yl)pyridazin-3-yl)- 1H-indazole | LCMS m/z = 568.3 (M + H); 1HNMR (400 MHz, DMSO-d6) δ 13.20 (s, 1H), 8.56 (s, 2H), 7.97 (d, J = 9.2 Hz, 1H), 7.94 (d, J = 2.4 Hz, 1H), 7.47 (d, J = 8.8 Hz, 1H), 7.09 (dd, J = 9.2 Hz, 2.4 Hz, 1H), 6.91 (d, J = 8.8 Hz, 1H), 6.08 (q, J = 6.8 Hz, 1H), 4.06 (d, J = 8.4 Hz, 2H), 3.97 (d, J = 8.8 Hz, 2H), 3.53 (t, J = 6.0 Hz, 2H), 2.61 (t, J = 6.0 Hz, 2H), 2.49-2.45 (m, 4H), 1.77 (d, J = 6.4 Hz, 3H), 1.69- 1.64 (m, 4H), 1.55 (s, 3H) |
| Example 270. | 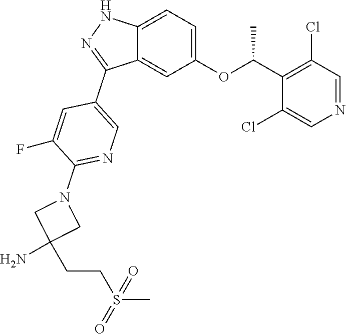 [SMILES: C[C@@H](Oc1ccc2[nH]nc(-c3cnc(N4CC(N)(CCS(C)(=O)=O)C4)c(F)c3)c2c1)c1c(Cl)cncc1Cl] | (R)-1-(5-(5-(1-(3,5- dichloropyridin-4- yl)ethoxy)-1H- indazol-3-yl)-3- fluoropyridin-2-yl)- 3-(2-(methyl- sulfonyl)ethyl) azetidin-3-amine | LCMS m/z = 579.4 (M + H); 1HNMR (400 MHz, DMSO-d6) δ 13.11 (s, 1H), 8.57 (s, 2H), 8.37 (t, J = 1.2 Hz, 1H), 7.70 (dd, J = 13.2 Hz, 2.0 Hz, 1H), 7.47 (d, J = 8.8 Hz, 1H), 7.20 (d, J = 2.0 Hz, 1H), 7.10 (dd, J = 8.8 Hz, 2.4 Hz, 1H), 6.14 (q, J = 6.8 Hz, 1H), 4.04 (dd, J = 8.8 Hz, 1.6 Hz, 2H), 3.86 (d, J = 7.6 Hz, 2H), 3.30-3.25 (m, 2H), 3.02 (s, 3H), 2.25 (brs, 2H), 2.18-2.11 (m, 2H), 1.76 (d, J = 6.8 Hz, 3H) |
| Example 271. | 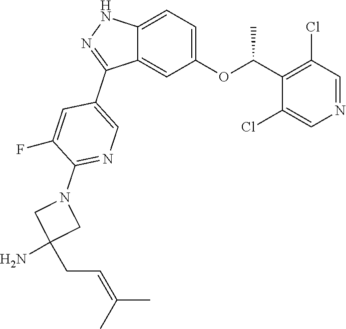 [SMILES: CC(C)=CCC1(N)CN(c2ncc(-c3n[nH]c4ccc(O[C@H](C)c5c(Cl)cncc5Cl)cc34)cc2F)C1] | (R)-1-(5-(5-(1-(3,5- dichloropyridin-4- yl)ethoxy)-1H- indazol-3-yl)-3- fluoropyridin-2-yl)- 3-(3-methylbut-2- en-1-yl)azetidin-3- amine | LCMS m/z = 541.3 (M + H); 1HNMR (400 MHz, DMSO-d6) δ 13.10 (brs, 1H), 8.57 (s, 2H), 8.36 (s, 1H), 7.69 (dd, J = 13.2 Hz, 1.6 Hz, 1H), 7.47 (d, J = 8.8 Hz, 1H), 7.19 (s, 1H), 7.11 (d, J = 2.4 Hz, 1H), 6.14 (q, J = 6.8 Hz, 1H), 5.31 (t, J = 7.6 Hz, 1H), 3.97 (d, J = 7.2 Hz, 2H), 3.86 (d, J = 7.2 Hz, 2H), 2.41 (d, J = 7.6 Hz, 2H), 1.76 (d, J = 6.4 Hz, 3H), 1.72 (s, 3H), 1.66 (s, 3H) |
| Example 272. | 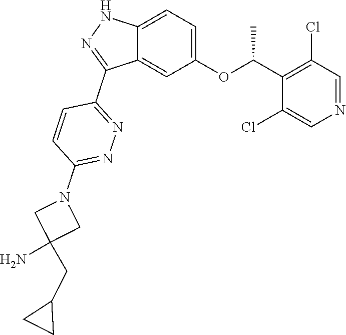 [SMILES: C[C@@H](Oc1ccc2[nH]nc(-c3ccc(N4CC(N)(CC5CC5)C4)nn3)c2c1)c1c(Cl)cncc1Cl] | (R)-3-(cyclo- propylmethyl)- 1-(6-(5-(1-(3,5- dichloropyridin-4- yl)ethoxy)-1H- indazol-3- yl)pyridazin-3- yl)azetidin-3-amine | LCMS m/z = 510.3 (M + H); 1HNMR (400 MHz, DMSO-d6) δ 13.13 (s, 1H), 8.52 (s, 2H), 7.93-7.91 (m, 2H), 7.43 (d, J = 9.2 Hz, 1H), 7.05 (dd, J = 8.8 Hz, 2.4 Hz, 1H), 6.84 (d, J = 9.3 Hz, 1H), 6.04 (q, J = 6.4 Hz, 1H), 4.05 (d, J = 8.0 Hz, 2H), 3.80 (dd, J = 8.4 Hz, 2.4 Hz, 2H), 1.73 (d, J = 6.4 Hz, 3H), 1.61 (d, J = 6.8 Hz, 2H), 0.87- 0.81 (m, 1H), 0.45-0.41 (m, 2H), 0.14-0.10 (m, 2H) |
| Example 273. | 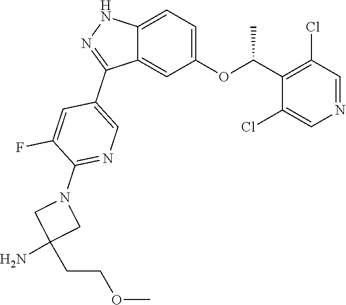 [SMILES: COCCC1(N)CN(c2ncc(-c3n[nH]c4ccc(O[C@H](C)c5c(Cl)cncc5Cl)cc34)cc2F)C1] | (R)-1-(5-(5-(1-(3,5- dichloropyridin-4- yl)ethoxy)-1H- indazol-3-yl)-3- fluoropyridin-2-yl)- 3-(2-methoxy- ethyl)azetidin- 3-amine | LCMS m/z = 531.3 (M + H); 1HNMR (400 MHz, DMSO-d6) δ 13.10 (s, 1H), 8.57 (s, 2H), 8.36 (t, J = 1.6 Hz, 1H), 7.68 (dd, J = 13.2 Hz, 1.6 Hz, 1H), 7.47 (d, J = 8.8 Hz, 1H), 7.19 (d, J = 2.0 Hz, 1H), 7.10 (dd, J = 9.2 Hz, 2.4 Hz, 1H), 6.14 (q, J = 6.4 Hz, 1H), 4.05 (dd, J = 8.8 Hz, 2.0 Hz, 2H), 3.85 (d, J = 7.2 Hz, 2H), 3.55 (t, J = 6.8 Hz, 2H), 3.26 (s, 3H), 1.97 (t, J = 6.4 Hz, 2H), 1.76 (d, J = 6.8 Hz, 3H) |
| Example 274. | 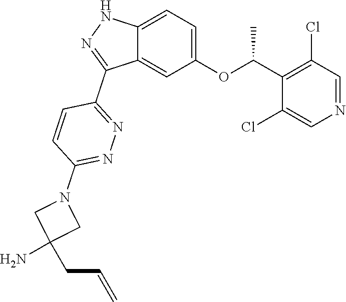 | (R)-3-allyl-1-(6-(5- (1-(3,5- dichloropyridin-4- yl)ethoxy)-1H- indazol-3- yl)pyridazin-3- yl)azetidin-3-amine | LCMS m/z = 496.3 (M + H); 1HNMR (400 MHz, DMSO-d6) δ 13.18 (s, 1H), 8.56 (s, 2H), 7.97 (d, J = 8.8 Hz, 1H), 7.94 (d, J = 2.4 Hz, 1H), 7.47 (d, J = 9.2 Hz, 1H), 7.09 (dd, J = 9.2 Hz, 2.8 Hz, 1H), 6.90 (d, J = 9.6 Hz, 1H), 6.08 (q, J = 6.8 Hz, 1H), 6.03-5.92 (m, 1H), 5.23 (dd, J =17.2 Hz, 2.4 Hz, 1H), 5.17 (dd, J = 10.0 Hz, 2.4 Hz, 1H), 4.03 (d, J = 8.0 Hz, 2H), 3.84 (d, J = 8.0 Hz, 2H), 2.54- 2.52 (m, 2H), 1.77 (d, J = 6.4 Hz, 3H) |
| Example 275. | 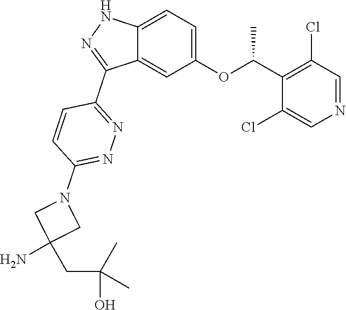 [SMILES: C[C@@H](Oc1ccc2[nH]nc(-c3ccc(N4CC(N)(CC(C)(C)O)C4)nn3)c2c1)c1c(Cl)cncc1Cl] | (R)-1-(3-amino-1- (6-(5-(1-(3,5- dichloropyridin-4- yl)ethoxy)-1H- indazol-3- yl)pyridazin-3- yl)azetidin-3-yl)-2- methylpropan-2-ol | LCMS m/z = 528.3 (M + H); 1HNMR (400 MHz, DMSO-d6) δ 13.17 (brs, 1H), 8.56 (s, 2H), 7.98-7.92 (m, 2H), 7.46 (d, J = 8.8 Hz, 1H), 7.09 (dd, J = 9.2 Hz, 2.4 Hz, 1H), 6.87 (d, J = 8.8 Hz, 1H), 6.08 (q, J = 6.8 Hz, 1H), 4.10 (d, J = 7.6 Hz, 2H), 3.88 (dd, J = 8.4 Hz, 2.8 Hz, 2H), 1.90 (s, 2H), 1.77 (d, J = 6.4 Hz, 3H), 1.19 (s, 6H) |
| Example 276. | 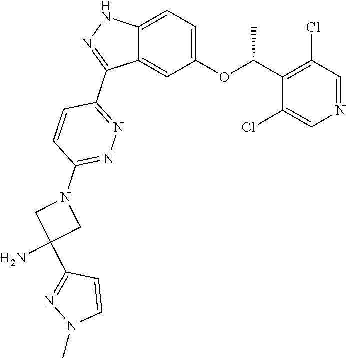 [SMILES: C[C@@H](Oc1ccc2[nH]nc(-c3ccc(N4CC(N)(c5ccn(C)n5)C4)nn3)c2c1)c1c(Cl)cncc1Cl] | (R)-1-(6-(5-(1-(3,5- dichloropyridin-4- yl)ethoxy)-1H- indazol-3- yl)pyridazin-3-yl)-3- (1-methyl-1H- pyrazol-4- yl)azetidin-3-amine | LCMS m/z = 536.3 (M+ H); 1HNMR (400 MHz, DMSO-d6) δ 13.20 (s, 1H), 8.56 (s, 2H), 8.00 (d, J = 8.8 Hz, 1H), 7.95 (s, 1H), 7.79 (s, 1H), 7.54 (s, 1H), 7.48 (d, J = 9.2 Hz, 1H), 7.11 (dd, J = 9.2 Hz, 2.4 Hz, 1H), 6.95 (d, J = 9.2 Hz, 1H), 6.11 (q, J = 6.4 Hz, 1H), 4.25- 4.22 (m, 2H), 4.16-4.12 (m, 2H), 3.82 (s, 3H), 1.78 (d, J = 6.8 Hz, 3H) |
| Example 277. | 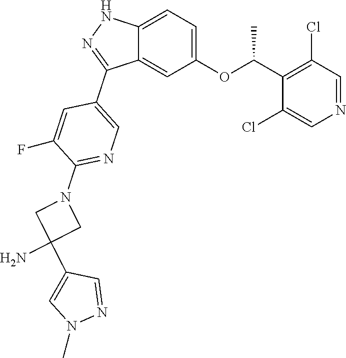 [SMILES: C[C@@H](Oc1ccc2[nH]nc(-c3cnc(N4CC(N)(c5cnn(C)c5)C4)c(F)c3)c2c1)c1c(Cl)cncc1Cl] | (R)-1-(5-(5-(1-(3,5- dichloropyridin-4- yl)ethoxy)-1H- indazol-3-yl)-3- fluoropyridin-2-yl)- 3-(1-methyl-1H- pyrazol-4- yl)azetidin-3-amine | LCMS m/z = 553.2 (M + H); 1HNMR (400 MHz, DMSO-d6) δ 13.12 (s, 1H), 8.58 (s, 2H), 8.40 (t, J = 1.6 Hz, 1H), 7.78 (s, 1H), 7.74 (dd, J = 13.2 Hz, 1.6 Hz, 1H), 7.53 (s, 1H), 7.49 (d, J = 9.2 Hz, 1H), 7.21 (d, J = 2.0 Hz, 1H), 7.11 (dd, J = 8.8 Hz, 2.0 Hz, 1H), 6.11 (q, J = 6.8 Hz, 1H), 4.22 (dd, J = 8.4 Hz, 2.0 Hz, 2H), 4.17 (d, J = 7.6 Hz, 2H), 3.82 (s, 3H), 1.77 (d, J = 6.8 Hz, 3H) |
| Example 278. | 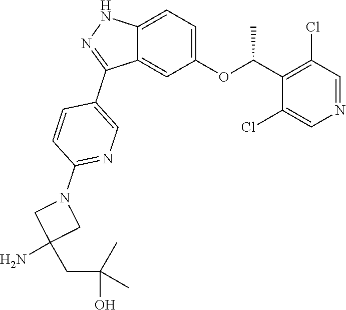 [SMILES: C[C@@H](Oc1ccc2[nH]nc(-c3ccc(N4CC(N)(CC(C)(C)O)C4)nc3)c2c1)c1c(Cl)cncc1Cl] | (R)-1-(3-amino-1- (5-(5-(1-(3,5- dichloropyridin-4- yl)ethoxy)-1H- indazol-3-yl)pyridin- 2-yl)azetidin-3-yl)- 2-methylpropan-2-ol | LCMS m/z = 527.3 (M + H); 1HNMR (400 MHz, DMSO-d6) δ 13.00 (brs, 1H), 8.60 (s, 2H), 8.51 (d, J = 2.4 Hz, 1H), 7.84 (dd, J = 8.4 Hz, 2.0 Hz, 1H), 7.45 (d, J = 9.2 Hz, 1H), 7.15 (d, J = 2.0 Hz, 1H), 7.10 (dd, J = 9.2 Hz, 2.4 Hz, 1H), 6.55 (d, J = 8.8 Hz, 1H), 6.11 (q, J = 6.4 Hz, 1H), 5.00 (brs, 1H), 4.03 (d, J = 8.0 Hz, 2H), 3.88 (d, J = 8.4 Hz, 2H), 1.93 (s, 2H), 1.76 (d, J = 6.8 Hz, 3H), 1.20 (s, 6H) |
| Example 279. | 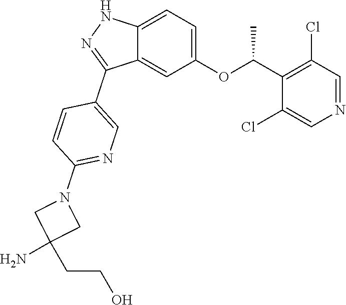 [SMILES: C[C@@H](Oc1ccc2[nH]nc(-c3ccc(N4CC(N)(CCO)C4)nc3)c2c1)c1c(Cl)cncc1Cl] | (R)-2-(3-amino-1- (5-(5-(1-(3,5- dichloropyridin-4- yl)ethoxy)-1H- indazol-3-yl)pyridin- 2-yl)azetidin-3- yl)ethanol | LCMS m/z = 499.2 (M + H); 1HNMR (400 MHz, DMSO-d6) δ 12.99 (brs, 1H), 8.60 (s, 2H), 8.50 (d, J = 2.0 Hz, 1H), 7.83 (dd, J = 8.4 Hz, 2.0 Hz, 1H), 7.45 (d, J = 9.2 Hz, 1H), 7.16 (d, J = 2.0 Hz, 1H), 7.09 (dd, J = 8.8 Hz, 1.6 Hz, 1H), 6.52 (d, J = 8.8 Hz, 1H), 6.11 (q, J = 6.8 Hz, 1H), 3.97 (d, J = 8.0 Hz, 2H), 3.91 (brs, 1H), 3.74 (d, J = 8.4 Hz, 2H), 3.65 (t, J = 6.4 Hz, 2H), 1.91 (t, J = 6.4 Hz, 2H), 1.76 (d, J = 6.4 Hz, 3H) |
| Example 280. | 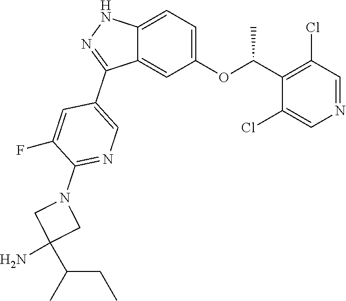 [SMILES: CCC(C)C1(N)CN(c2ncc(-c3n[nH]c4ccc(O[C@H](C)c5c(Cl)cncc5Cl)cc34)cc2F)C1] | 1-[5-[5-[(1R)-1-(3,5- dichloro-4- pyridyl)ethoxy]-1H- indazol-3-yl]-3- fluoro-2-pyridyl]-3- sec-butyl-azetidin-3- amine | LCMS m/z = 529.1 (M + H); 1HNMR (400 MHz, DMSO-d6) δ 13.10 (br s, 1H), 8.57 (s, 2H), 8.35 (s, 1H), 7.67 (dd, J = 1.7, 13.3 Hz, 1H), 7.47 (d, J = 8.9 Hz, 1H), 7.19 (d, J = 2.0 Hz, 1H), 7.10 (dd, J = 2.2, 9.0 Hz, 1H), 6.14 (q, J = 6.6 Hz, 1H), 4.05-3.99 (m, 2H), 3.84-3.76 (m, 2H), 1.96 (br s, 2H), 1.76 (d, J = 6.6 Hz, 3H), 1.68-1.58 (m, 1H), 1.54-1.43 (m, 1H), 1.18-1.08 (m, 1H), 0.94-0.87 (m, 6H) |
| Example 281. | 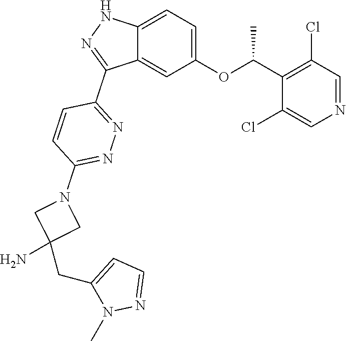 [SMILES: C[C@@H](Oc1ccc2[nH]nc(-c3ccc(N4CC(N)(Cc5ccnn5C)C4)nn3)c2c1)c1c(Cl)cncc1Cl] | (R)-1-(6-(5-(1-(3,5- dichloropyridin-4- yl)ethoxy)-1H- indazol-3- yl)pyridazin-3-yl)-3- ((1-methyl-1H- pyrazol-5- yl)methyl)azetidin- 3-amine | LCMS m/z = 550.3 (M + H); 1HNMR (400 MHz, DMSO-d6) δ 13.17 (s, 1H), 8.56 (s, 2H), 7.97 (d, J = 9.2 Hz, 1H), 7.94 (d, J = 2.0 Hz, 1H), 7.47 (d, J = 8.8 Hz, 1H), 7.33 (d, J = 2.0 Hz, 1H), 7.09 (dd, J = 8.8 Hz, 2.4 Hz, 1H), 6.91 (d, J = 9.2 Hz, 1H), 6.23 (d, J = 2.0 Hz, 1H), 6.08 (q, J = 6.8 Hz, 1H), 4.11 (d, J = 8.0 Hz, 2H), 3.86 (d, J = 8.4 Hz, 2H), 3.82 (s, 3H), 3.13 (s, 2H), 1.77 (d, J = 6.4 Hz, 3H) |
| Example 282. | 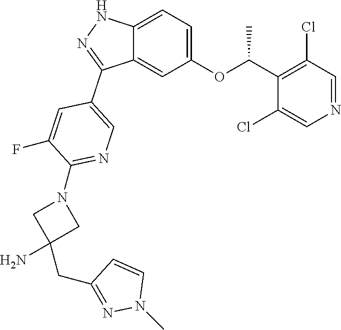 [SMILES: C[C@@H](Oc1ccc2[nH]nc(-c3cnc(N4CC(N)(Cc5ccn(C)n5)C4)c(F)c3)c2c1)c1c(Cl)cncc1Cl] | (R)-1-(5-(5-(1-(3,5- dichloropyridin-4- yl)ethoxy)-1H- indazol-3-yl)-3- fluoropyridin-2-yl)- 3-((1-methyl-1H- pyrazol-3- yl)methyl)azetidin- 3-amine | LCMS m/z = 567.3 (M + H); 1HNMR (400 MHz, DMSO-d6) δ 13.10 (s, 1H), 8.57 (s, 2H), 8.35 (s, 1H), 7.68 (dd, J = 13.2 Hz, 1.6 Hz, 1H), 7.58 (d, J = 2.0 Hz, 1H), 7.47 (d, J = 9.2 Hz, 1H), 7.19 (d, J = 2.0 Hz, 1H), 7.10 (dd, J = 9.2 Hz, 2.0 Hz, 1H), 6.17 (d, J = 2.4 Hz, 1H), 6.13 (q, J = 7.2 Hz, 1H), 4.12 (d, J = 8.8 Hz, 2H), 3.85 (d, J = 7.6 Hz, 2H), 3.79 (s, 3H), 2.96 (s, 2H), 1.76 (d, J = 6.4 Hz, 3H) |
| Example 283. | 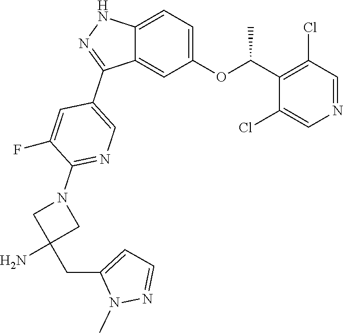 [SMILES: C[C@@H](Oc1ccc2[nH]nc(-c3cnc(N4CC(N)(Cc5ccnn5C)C4)c(F)c3)c2c1)c1c(Cl)cncc1Cl] | (R)-1-(5-(5-(1-(3,5- dichloropyridin-4- yl)ethoxy)-1H- indazol-3-yl)-3- fluoropyridin-2-yl)- 3-((1-methyl-1H- pyrazol-5- yl)methyl)azetidin- 3-amine | LCMS m/z = 567.3 (M + H); 1HNMR (400 MHz, DMSO-d6) δ 13.10 (s, 1H), 8.57 (s, 2H), 8.37 (t, J = 1.6 Hz, 1H), 7.70 (dd, J = 13.2 Hz, 2.0 Hz, 1H), 7.47 (d, J = 8.0 Hz, 1H), 7.33 (d, J = 2.0 Hz, 1H), 7.20 (d, J = 2.0 Hz, 1H), 7.10 (dd, J = 9.2 Hz, 2.4 Hz, 1H), 6.22 (s, 1H), 6.14 (q, J = 6.8 Hz, 1H), 4.12 (d, J = 8.0 Hz, 2H), 3.93 (d, J = 8.0 Hz, 2H), 3.81 (s, 3H), 3.13 (s, 2H), 1.76 (d, J = 6.4 Hz, 3H) |
| Example 284. | 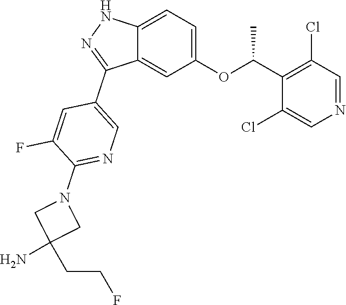 [SMILES: C[C@@H](Oc1ccc2[nH]nc(-c3cnc(N4CC(N)(CCF)C4)c(F)c3)c2c1)c1c(Cl)cncc1Cl] | (R)-1-(5-(5-(1-(3,5- dichloropyridin-4- yl)ethoxy)-1H- indazol-3-yl)-3- fluoropyridin-2-yl)- 3-(2- fluoroethyl)azetidin- 3-amine | LCMS m/z = 519.4 (M + H); 1HNMR (400 MHz, DMSO-d6) δ 13.11 (s, 1H), 8.57 (s, 2H), 8.37 (s, 1H), 7.69 (dd, J = 13.2 Hz, 1.6 Hz, 1H), 7.47 (d, J = 9.2 Hz, 1H), 7.19 (d, J = 1.6 Hz, 1H), 7.10 (dd, J = 9.2 Hz, 2.4 Hz, 1H), 6.14 (q, J = 6.4 Hz, 1H), 4.70 (dt, J = 47.2 Hz, 6.4 Hz, 2H), 4.07 (d, J = 6.4 Hz, 2H), 3.87 (d, J = 7.6 Hz, 2H), 2.34-2.25 (m, 2H), 2.14 (dt, J = 24.4 Hz, 6.4 Hz, 2H), 1.76 (d, J = 6.4 Hz, 3H) |
| Example 285. | 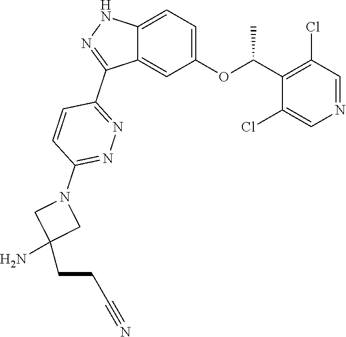 | (R)-3-(3-amino-1- (6-(5-(1-(3,5- dichloropyridin-4- yl)ethoxy)-1H- indazol-3- yl)pyridazin-3- yl)azetidin-3- yl)propanenitrile | LCMS m/z = 509.4 (M + H); 1HNMR (400 MHz, DMSO-d6) δ 13.18 (s, 1H), 8.56 (s, 2H), 7.97 (d, J = 9.2 Hz, 1H), 7.94 (d, J = 2.4 Hz, 1H), 7.47 (d, J = 8.8 Hz, 1H), 7.09 (dd, J = 8.8 Hz, 2.4 Hz, 1H), 6.88 (d, J = 9.2 Hz, 1H), 6.08 (q, J = 6.4 Hz, 1H), 4.06 (d, J =8.4 Hz, 2H), 3.82 (d, J = 8.0 Hz, 2H), 2.64 (t, J = 8.0 Hz, 2H), 2.09 (t, J = 7.2 Hz, 2H), 1.77 (d, J = 6.8 Hz, 3H |
| Example 286. | 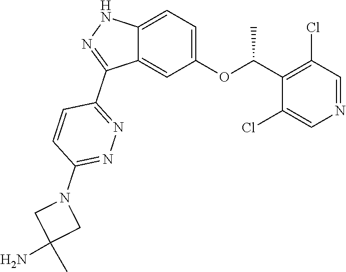 [SMILES: C[C@@H](Oc1ccc2[nH]nc(-c3ccc(N4CC(C)(N)C4)nn3)c2c1)c1c(Cl)cncc1Cl] | R)-1-(6-(5-(1-(3,5- dichloropyridin-4- yl)ethoxy)-1H- indazol-3- yl)pyridazin-3-yl)-3- methylazetidin-3- amine | LCMS m/z = 470.2 (M + H); 1HNMR (400 MHz, DMSO-d6) δ 13.18 (brs, 1H), 8.55 (s, 2H), 7.96-7.94 (m, 2H), 7.46 (d, J = 8.8 Hz, 1H), 7.08 (dd, J = 8.8 Hz, 2.4 Hz, 1H), 6.87 (d, J = 9.2 Hz, 1H), 6.07 (q, J = 6.4 Hz, 1H), 3.93 (d, J = 8.0 Hz, 2H), 3.86 (d, J = 7.2 Hz, 2H), 1.76 (d, J = 6.4 Hz, 3H), 1.45 (s, 3H) |
| Example 287. | 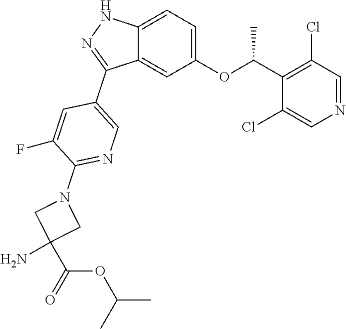 [SMILES: CC(C)OC(=O)C1(N)CN(c2ncc(-c3n[nH]c4ccc(O[C@H](C)c5c(Cl)cncc5Cl)cc34)cc2F)C1] | (R)-isopropyl 3- amino-1-(5-(5-(1- (3,5-dichloropyridin- 4-yl)ethoxy)-1H- indazol-3-yl)-3- fluoropyridin-2- yl)azetidine-3- carboxylate | LCMS m/z = 559.5 (M + H); 1HNMR (400 MHz, DMSO-d6) δ 13.13 (s, 1H), 8.58 (s, 2H), 8.39-8.39 (m, 1H), 7.74 (dd, J = 12.8 Hz, 1.6 Hz, 1H), 7.48 (d, J = 8.8 Hz, 1H), 7.21 (d, J = 1.6 Hz, 1H), 7.10 (dd, J = 9.2 Hz, 2.4 Hz, 1H), 6.15 (q, J = 6.0 Hz, 1H), 5.03-4.97 (m, 1H), 4.40 (d, J = 8.0 Hz, 2H), 4.02 (d, J = 8.0 Hz, 2H), 1.76 (d, J = 6.8 Hz, 3H), 1.25 (d, J = 6.0 Hz, 6H) |
| Example 288. | 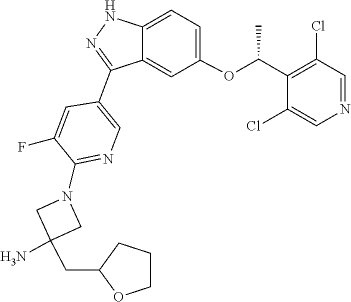 | 1-(5-(5-((R)-1-(3,5- dichloropyridin-4- yl)ethoxy)-1H- indazol-3-yl)-3- fluoropyridin-2-yl)- 3-((tetrahydrofuran- 2- yl)methyl)azetidin- 3-amine | LCMS m/z = 557.2 (M + H); 1HNMR (400 MHz, DMSO-d6) δ 13.11 (s, 1H), 8.57 (s, 2H), 8.35 (s, 1H), 7.70 (d, J = 13.6 Hz, 1H), 7.48 (d, J = 9.2 Hz, 1H), 7.18 (s, 1H), 7.11 (dd, J = 8.8 Hz, 1.6 Hz, 1H), 6.16 (q, J = 6.4 Hz, 1H), 4.13-3.99 (m, 3H), 3.90-3.76 (m, 3H), 3.65-3.55 (m, 1H), 2.07-1.78 (m, 5H), 1.77 (d, J = 6.4 Hz, 3H), 1.51- 1.42 (m,1H) |
| Example 289. | 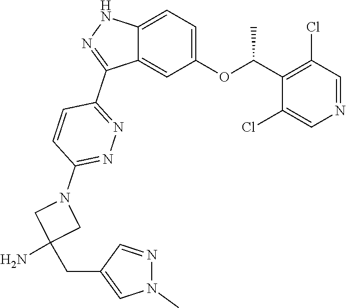 [SMILES: C[C@@H](Oc1ccc2[nH]nc(-c3ccc(N4CC(N)(Cc5cnn(C)c5)C4)nn3)c2c1)c1c(Cl)cncc1Cl] | (R)-1-(6-(5-(1-(3,5- dichloropyridin-4- yl)ethoxy)-1H- indazol-3- yl)pyridazin-3-yl)-3- ((1-methyl-1H- pyrazol-4- yl)methyl)azetidin- 3-amine | LCMS m/z = 550.3 (M + H); 1HNMR (400 MHz, DMSO-d6) δ 13.19 (s, 1H), 8.56 (s, 2H), 7.97-7.92 (m, 2H), 7.64 (s, 1H), 7.47 (d, J = 9.2 Hz, 1H), 7.39 (s, 1H), 7.09 (dd, J = 8.8 Hz, 2.4 Hz, 1H), 6.87 (d, J = 9.2 Hz, 1H), 6.08 (q, J = 6.8 Hz, 1H), 4.05 (d, J = 8.4 Hz, 2H), 3.85- 3.77 (m, 5H), 2.85 (s, 2H), 1.77 (d, J = 6.8 Hz, 3H) |
| Example 290. | 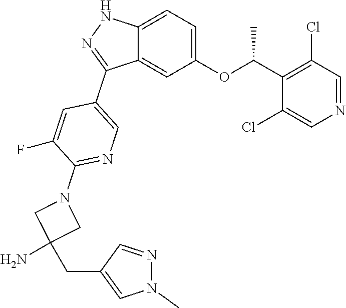 [SMILES: C[C@@H](Oc1ccc2[nH]nc(-c3cnc(N4CC(N)(Cc5cnn(C)c5)C4)c(F)c3)c2c1)c1c(Cl)cncc1Cl] | (R)-1-(5-(5-(1-(3,5- dichloropyridin-4- yl)ethoxy)-1H- indazol-3-yl)-3- fluoropyridin-2-yl)- 3-((1-methyl-1H- pyrazol-4- yl)methyl)azetidin- 3-amine | LCMS m/z = 567.3 (M + H); 1HNMR (400 MHz, DMSO-d6) δ 13.11 (s, 1H), 8.57 (s, 2H), 8.35 (s, 1H), 7.68 (dd, J = 13.2 Hz, 1.6 Hz, 1H), 7.63 (s, 1H), 7.47 (d, J = 9.2 Hz, 1H), 7.37 (s, 1H), 7.19 (s, 1H), 7.10 (dd, J = 9.2 Hz, 2.4 Hz, 1H), 6.14 (q, J = 6.4 Hz, 1H), 4.04 (d, J = 7.6 Hz, 2H), 3.84 (d, J = 8.4 Hz, 2H), 3.79 (s, 3H), 2.83 (s, 2H), 1.76 (d, J = 6.4 Hz, 3H) |
| Example 291. | 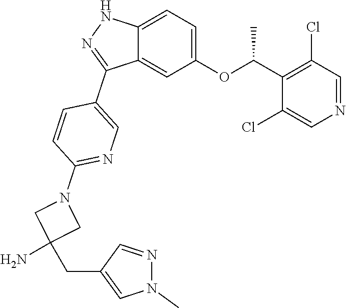 [SMILES: C[C@@H](Oc1ccc2[nH]nc(-c3ccc(N4CC(N)(Cc5cnn(C)c5)C4)nc3)c2c1)c1c(Cl)cncc1Cl] | (R)-1-(5-(5-(1-(3,5- dichloropyridin-4- yl)ethoxy)-1H- indazol-3-yl)pyridin- 2-yl)-3-((1-methyl- 1H-pyrazol-3- yl)methyl)azetidin- 3-amine | LCMS m/z = 549.6 (M + H); 1HNMR (400 MHz, DMSO-d6) δ 13.00 (s, 1H), 8.60 (s, 2H), 8.50 (d, J = 2.0 Hz, 1H), 7.84 (dd, J = 8.4 Hz, 2.0 Hz, 1H), 7.62 (d, J= 2.0 Hz, 1H), 7.46 (d, J = 9.2 Hz, 1H), 7.15 (d, J= 1.6 Hz, 1H), 7.10 (dd, J = 6.8 Hz, 2.0 Hz, 1H), 6.56 (d, J = 8.4 Hz, 1H), 6.21 (d, J = 2.0 Hz, 1H), 6.11 (q, J = 6.8 Hz, 1H), 4.07 (d, J = 8.4 Hz, 2H), 3.84 (d, J = 8.4 Hz, 2H), 3.80 (s, 3H), 3.04 (s, 2H), 1.76 (d, J = 6.4 Hz, 3H) |
| Example 292. | 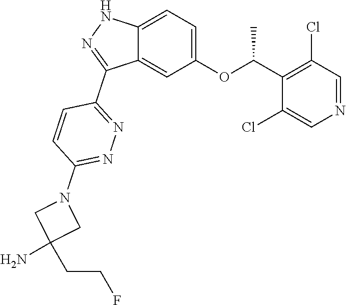 [SMILES: C[C@@H](Oc1ccc2[nH]nc(-c3ccc(N4CC(N)(CCF)C4)nn3)c2c1)c1c(Cl)cncc1Cl] | (R)-1-(6-(5-(1-(3,5- dichloropyridin-4- yl)ethoxy)-1H- indazol-3- yl)pyridazin-3-yl)-3- (2- fluoroethyl)azetidin- 3-amine | LCMS m/z = 502.5 (M + H); 1HNMR (400 MHz, DMSO-d6) δ 13.18 (s, 1H), 8.56 (s, 2H), 7.98-7.95 (m, 2H), 7.46 (d, J = 9.2 Hz, 1H), 7.09 (dd, J = 8.8 Hz, 2.4 Hz, 1H), 6.90 (d, J = 9.2 Hz, 1H), 6.08 (q, J = 6.4 Hz, 1H), 4.70 (dt, J = 47.2 Hz, 6.4 Hz, 2H), 4.07 (d, J = 8.0 Hz, 2H), 3.85 (d, J = 8.0 Hz, 2H), 3.02-2.73 (m, 2H), 2.14 (dt, J = 24.4 Hz, 6.0 Hz, 2H), 1.77 (d, J = 6.4 Hz, 3H) |
| Example 293. | 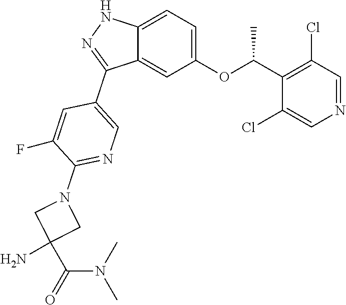 [SMILES: C[C@@H](Oc1ccc2[nH]nc(-c3cnc(N4CC(N)(C(=O)N(C)C)C4)c(F)c3)c2c1)c1c(Cl)cncc1Cl] | (R)-3-amino-1-(5- (5-(1-(3,5- dichloropyridin-4- yl)ethoxy)-1H- indazol-3-yl)-3- fluoropyridin-2-yl)- N,N- dimethylazetidine-3- carboxamide | LCMS m/z = 544.2 (M + H); 1HNMR (400 MHz, DMSO-d6) δ 13.12 (s, 1H), 8.57 (s, 2H), 8.37 (t, J = 1.6 Hz, 1H), 7.71 (dd, J = 13.2 Hz, 2.0 Hz, 1H), 7.47 (d, J = 9.2 Hz, 1H), 7.19 (d, J = 1.6 Hz, 1H), 7.10 (dd, J = 8.8 Hz, 2.0 Hz, 1H), 6.14 (q, J = 6.4 Hz, 1H), 4.55 (d, J = 8.4 Hz, 1H), 3.98 (d, J = 8.4 Hz, 1H), 3.04 (s, 3H), 2.86 (s, 3H), 1.76 (d, J = 6.8 Hz, 3H) |
| Example 294. | 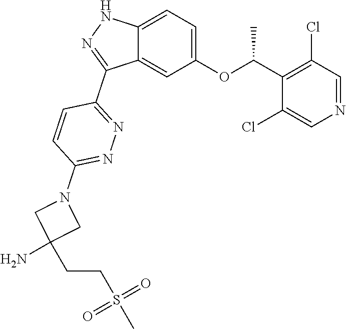 [SMILES: C[C@@H](Oc1ccc2[nH]nc(-c3ccc(N4CC(N)(CCS(C)(=O)=O)C4)nn3)c2c1)c1c(Cl)cncc1Cl] | (R)-1-(6-(5-(1-(3,5- dichloropyridin-4- yl)ethoxy)-1H- indazol-3- yl)pyridazin-3-yl)-3- (2-(methyl- sulfonyl)ethyl) azetidin-3-amine | LCMS m/z = 562.4 (M + H); 1HNMR (400 MHz, DMSO-d6) δ 13.19 (s, 1H), 8.56 (s, 2H), 7.97 (d, J = 9.2 Hz, 1H), 7.94 (d, J = 2.4 Hz, 1H), 7.47 (d, J = 9.2 Hz, 1H), 7.09 (dd, J = 8.8 Hz, 2.4 Hz, 1H), 6.88 (d, J = 9.6 Hz, 1H), 6.08 (q, J = 6.8 Hz, 1H), 4.03 (d, J = 8.4 Hz, 2H), 3.81 (d, J = 8.0 Hz, 2H), 3.32- 3.25 (m, 2H), 3.03 (s, 3H), 2.32 (brs, 2H), 2.20-2.12 (m, 2H), 1.77 (d, J = 6.8 Hz, 3H) |
| Example 295. | 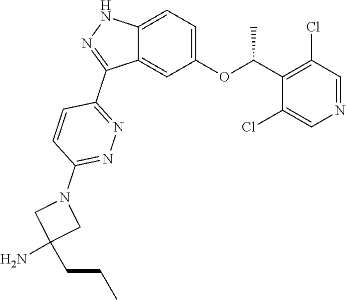 | (R)-1-(6-(5-(1-(3,5- dichloropyridin-4- yl)ethoxy)-1H- indazol-3- yl)pyridazin-3-yl)-3- propylazetidin-3- amine | LCMS m/z = 498.2 (M + H); 1HNMR (400 MHz, DMSO-d6) δ 13.18 (brs, 1H), 8.55 (s, 2H), 7.97-7.94 (m, 2H), 7.46 (d, J = 9.2 Hz, 1H), 7.09 (dd, J = 8.8 Hz, 2.0 Hz, 1H), 6.87 (d, J = 9.2 Hz, 1H), 6.08 (q, J = 6.4 Hz, 1H), 3.98 (d, J = 8.0 Hz, 2H), 3.81(d, J = 8.0 Hz, 2H), 1.77 (d, J = 6.0 Hz, 3H), 1.71-1.67 (m, 2H), 1.47-1.41 (m, 2H), 0.95 (t, J = 7.2 Hz, 3H) |
| Example 296. | 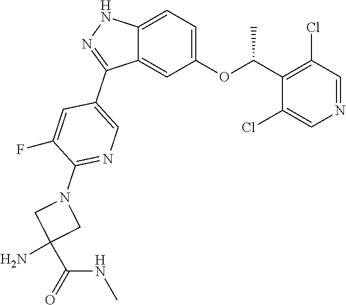 [SMILES: CNC(=O)C1(N)CN(c2ncc(-c3n[nH]c4ccc(O[C@H](C)c5c(Cl)cncc5Cl)cc34)cc2F)C1] | (R)-3-amino-1-(5- (5-(1-(3,5- dichloropyridin-4- yl)ethoxy)-1H- indazol-3-yl)-3- fluoropyridin-2-yl)- N-methylazetidine- 3-carboxamide | LCMS m/z = 530.3 (M + H); 1HNMR (400 MHz, DMSO-d6) δ 13.11 (s, 1H), 8.57 (s, 2H), 8.38 (s, 1H), 7.97-7.95 (m, 1H), 7.71 (dd, J = 13.2 Hz, 1.6 Hz, 1H), 7.47 (d, J = 9.2 Hz, 1H), 7.21 (s, 1H), 7.10 (dd, J = 9.2 Hz, 2.4 Hz, 1H), 6.15 (q, J = 6.4 Hz, 1H), 4.39 (d, J = 7.2 Hz, 2H), 3.92 (d, J = 6.8 Hz, 2H), 2.90 (brs, 2H), 2.67 (d, J = 4.8 Hz, 3H), 1.76 (d, J = 6.4 Hz, 3H) |
| Example 297. | 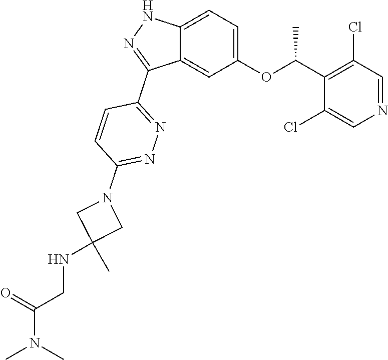 [SMILES: C[C@@H](Oc1ccc2[nH]nc(-c3ccc(N4CC(C)(NCC(=O)N(C)C)C4)nn3)c2c1)c1c(Cl)cncc1Cl] | R)-N-(1-(6-(5-(1- (3,5-dichloropyridin- 4-yl)ethoxy)-1H- indazol-3- yl)pyridazin-3-yl)-3- methylazetidin-3- yl)-2-(dimethyl- amino)acet- amide | LCMS m/z = 555.3 (M + H); 1HNMR (400 MHz, DMSO-d6) δ 13.26 (s, 1H), 9.00 (brs, 1H), 8.55 (s, 2H), 7.98 (d, J = 9.2 Hz, 1H), 7.93 (d, J = 2.0 Hz, 1H), 7.47 (d, J = 8.8 Hz, 1H), 7.09 (dd, J = 9.2 Hz, 2.8 Hz, 1H), 6.94 (d, J = 9.6 Hz, 1H), 6.07 (q, J = 6.8 Hz, 1H), 4.25 (d, J = 8.4 Hz, 2H), 4.05 (d, J = 8.4 Hz, 2H), 3.53 (s, 2H), 2.59 (s, 6H), 1.76 (d, J = 6.8 Hz, 3H), 1.63 (s, 3H) |
| Example 298. | 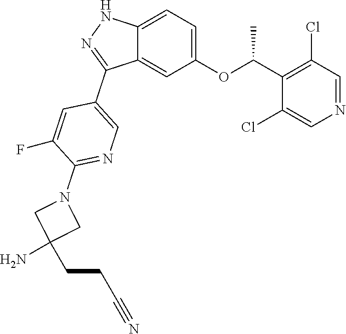 | (R)-3-(3-amino-1- (5-(5-(1-(3,5- dichloropyridin-4- yl)ethoxy)-1H- indazol-3-yl)-3- fluoropyridin-2- yl)azetidin-3- yl)propanenitrile | LCMS m/z = 526.3 (M + H); 1HNMR (400 MHz, DMSO-d6) δ 13.11 (s, 1H), 8.57 (s, 2H), 8.37 (t, J = 1.2 Hz, 1H), 7.70 (dd, J = 13.2 Hz, 2.0 Hz, 1H), 7.47 (d, J = 9.2 Hz, 1H), 7.20 (d, J = 1.6 Hz, 1H), 7.10 (dd, J = 9.2 Hz, 2.4 Hz, 1H), 6.14 (q, J = 6.4 Hz, 1H), 4.05 (d, J = 6.8 Hz, 2H), 3.85 (d, J = 7.6 Hz, 2H), 2.63 (t, J = 7.6 Hz, 2H), 2.06 (t, J = 7.6 Hz, 2H), 1.76 (d, J = 6.4 Hz, 3H) |
| Example 299. | 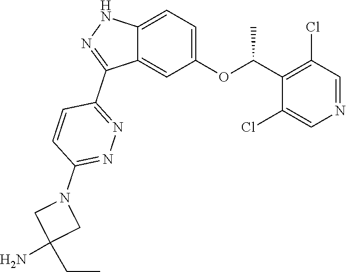 [SMILES: CCC1(N)CN(c2ccc(-c3n[nH]c4ccc(O[C@H](C)c5c(Cl)cncc5Cl)cc34)nn2)C1] | (R)-1-(6-(5-(1-(3,5- dichloropyridin-4- yl)ethoxy)-1H- indazol-3- yl)pyridazin-3-yl)-3- ethylazetidin-3- amine | LCMS m/z = 484.2 (M + H); 1HNMR (400 MHz, DMSO-d6) δ 13.16 (brs, 1H), 8.56 (s, 2H), 7.97-7.94 (m, 2H), 7.47 (d, J = 8.8 Hz, 1H), 7.09 (dd, J = 9.2 Hz, 2.8 Hz, 1H), 6.87 (d, J = 9.2 Hz, 1H), 6.08 (q, J = 6.8 Hz, 1H), 3.95 (d, J = 8.0 Hz, 2H), 3.78 (d, J = 8.0 Hz, 2H), 2.20 (brs, 2H), 1.78-1.70 (m, 5H), 0.97 (t, J = 7.6 Hz, 3H) |
| Example 300. | 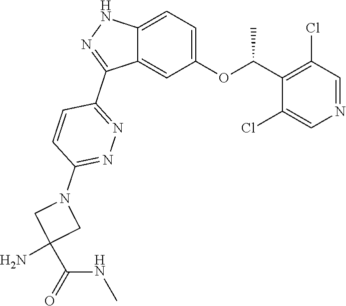 [SMILES: CNC(=O)C1(N)CN(c2ccc(-c3n[nH]c4ccc(O[C@H](C)c5c(Cl)cncc5Cl)cc34)nn2)C1] | (R)-3-amino-1-(6- (5-(1-(3,5- dichloropyridin-4- yl)ethoxy)-1H- indazol-3- yl)pyridazin-3-yl)- N-methylazetidine- 3-carboxamide | LCMS m/z = 513.2 (M + H); 1HNMR (400 MHz, DMSO-d6) δ 13.18 (s, 1H), 8.56 (s, 2H), 7.99-7.95 (m, 3H), 7.47 (d, J = 9.2 Hz, 1H), 7.09 (dd, J = 9.2 Hz, 2.4 Hz, 1H), 6.91 (d, J = 9.2 Hz, 1H), 6.08 (q, J = 6.8 Hz, 1H), 4.37 (d, J = 8.0 Hz, 2H), 3.89 (d, J = 8.0 Hz, 2H), 2.78 (brs, 2H), 2.67 (d, J = 4.8 Hz, 3H), 1.77 (d, J = 6.4 Hz, 3H) |
| Example 301. | 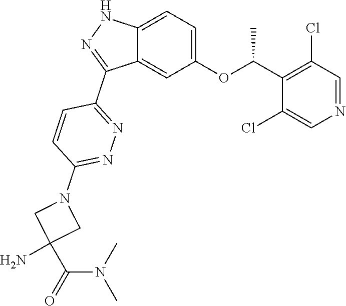 [SMILES: C[C@@H](Oc1ccc2[nH]nc(-c3ccc(N4CC(N)(C(=O)N(C)C)C4)nn3)c2c1)c1c(Cl)cncc1Cl] | (R)-3-amino-1-(6- (5-(1-(3,5- dichloropyridin-4- yl)ethoxy)-1H- indazol-3- yl)pyridazin-3-yl)- N,N- dimethylazetidine-3- carboxamide | LCMS m/z = 527.2 (M + H); 1HNMR (400 MHz, DMSO-d6) δ 13.19 (s, 1H), 8.60 (s, 2H), 7.97 (d, J = 9.2 Hz, 1H), 7.94 (d, J = 2.4 Hz, 1H), 7.47 (d, J = 9.2 Hz, 1H), 7.09 (dd, J = 9.2 Hz, 2.4 Hz, 1H), 6.93 (d, J = 9.6 Hz, 1H), 6.08 (q, J = 6.8 Hz, 1H), 4.54 (d, J = 8.4 Hz, 2H), 3.95 (dd, J = 8.4 Hz, 3.2 Hz, 2H), 3.06 (s, 3H), 2.87 (s, 3H), 1.77 (d, J = 6.4 Hz, 3H) |
| Example 302. | 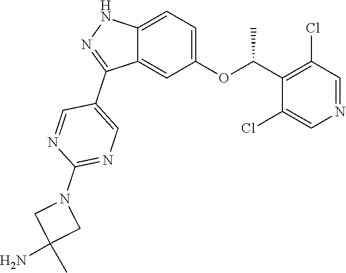 | (R)-1-(5-(5-(1-(3,5- dichloropyridin-4- yl)ethoxy)-6- methoxy-1H- indazol-3- yl)pyrimidin-2-yl)- 3-methylazetidin-3- amine | LCMS m/z = 500.3 (M + H); 1HNMR (400 MHz, DMSO-d6) δ 12.94 (brs, 1H), 8.69 (s, 2H), 8.59 (s, 2H), 7.14 (s, 1H), 7.00 (s, 1H), 6.01 (q, J = 6.8 Hz, 1H), 3.91-3.83 (m, 7H), 1.76 (d, J = 6.8 Hz, 3H), 1.40 (s, 3H) |
| Example 303. | 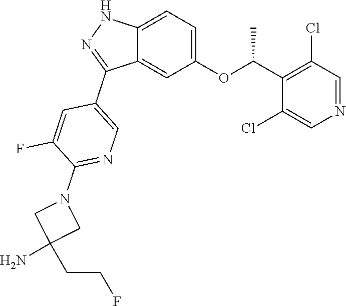 | (R)-1-(5-(5-(1-(3,5- dichloropyridin-4- yl)ethoxy)-1H- indazol-3-y1)-3- fluoropyridin-2-yl)- N-(2-fluoroethyl)-3- methylazetidin-3- amine | LCMS m/z = 533.2 (M + H); 1HNMR (400 MHz, DMSO-d6) δ 13.10 (s, 1H), 8.57 (s, 2H), 8.37 (s, 1H), 7.69 (dd, J = 13.2 Hz, 1.6 Hz, 1H), 7.47 (d, J = 9.2 Hz, 1H), 7.20 (d, J = 1.6 Hz, 1H), 7.10 (J = 8.8 Hz, 2.0 Hz, 1H), 6.14 (q, J = 6.4 Hz, 1H), 4.47 (dt, J = 47.2 Hz, 5.6 Hz, 2H), 4.01 (d, J = 8.4 Hz, 2H), 3.90 (d, J = 8.8 Hz, 2H), 2.85 (dt, J = 26.4 Hz, 5.2 Hz, 2H), 1.76 (d, J = 6.8 Hz, 3H), 1.45 (s, 3H) |
| Example 304. | 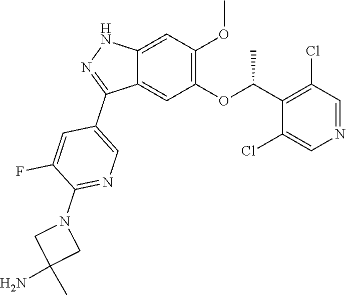 [SMILES: COc1cc2[nH]nc(-c3cnc(N4CC(C)(N)C4)c(F)c3)c2cc1O[C@H](C)c1c(Cl)cncc1Cl] | (R)-1-(5-(5-(1-(3,5- dichloropyridin-4- yl)ethoxy)-6- methoxy-1H- indazol-3-yl)-3- fluoropyridin-2-yl)- 3-methylazetidin-3- amine | LCMS m/z = 517.3 (M + H); 1HNMR (400 MHz, DMSO-d6) δ 12.98 (s, 1H), 8.58 (s, 2H), 8.30 (t, J = 1.6 Hz, 1H), 7.64 (dd, J = 13.2 Hz, 1.6 Hz, 1H), 7.08 (s, 1H), 7.00 (s, 1H), 5.98 (q, J = 6.8 Hz, 1H), 3.97-3.93 (m, 4H), 3.85 (s, 3H), 1.75 (d, J = 6.4 Hz, 3H), 1.46 (s, 3H) |
| Example 305. | 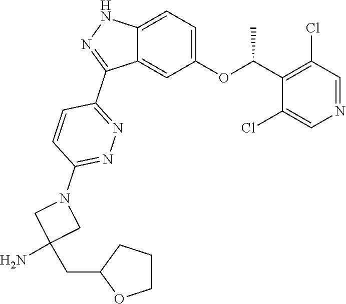 [SMILES: C[C@@H](Oc1ccc2[nH]nc(-c3ccc(N4CC(N)(CC5CCCO5)C4)nn3)c2c1)c1c(Cl)cncc1Cl] | 1-(6-(5-((R)-1-(3,5- dichloropyridin-4- yl)ethoxy)-1H- indazol-3- yl)pyridazin-3-yl)-3- ((tetrahydrofuran-2- yl)methyl)azetidin- 3-amine | LCMS m/z = 540.3 (M + H); 1HNMR (400 MHz, DMSO-d6) δ 13.19 (s, 1H), 8.56 (s, 2H), 7.98-7.93 (m, 2H), 7.48 (d, J = 9.2 Hz, 1H), 7.11 (dd, J = 9.2 Hz, 2.4 Hz 1H), 6.92-6.90 (m, 1H), 6.10 (q, J = 6.8 Hz, 1H), 4.16-4.12 (m, 2H), 4.11-4.01 (m, 1H), 3.93-3.88 (m, 2H), 3.82 (q, J = 7.6 Hz, 1H), 3.67- 3.62 (m, 1H), 2.08-1.97 (m, 3H), 1.91-1.76 (m, 5H), 1.53- 1.44 (m, 1H) |
| Example 306. | 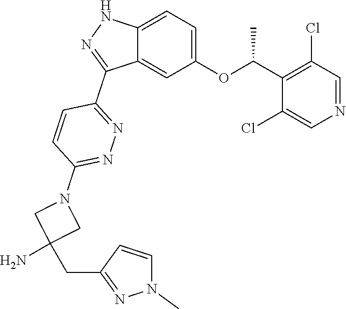 [SMILES: C[C@@H](Oc1ccc2[nH]nc(-c3ccc(N4CC(N)(Cc5ccn(C)n5)C4)nn3)c2c1)c1c(Cl)cncc1Cl] | (R)-1-(6-(5-(1-(3,5- dichloropyridin-4- yl)ethoxy)-1H- indazol-3- yl)pyridazin-3-yl)-3- ((1-methyl-1H- pyrazol-3- yl)methyl)azetidin- 3-amine | LCMS m/z = 550.2 (M + H); 1HNMR (400 MHz, DMSO-d6) δ 13.18 (s, 1H), 8.56 (s, 2H), 7.96-7.94 (m, 2H), 7.59 (d, J = 2.0 Hz, 1H), 7.47 (d, J = 9.2 Hz, 1H), 7.09 (dd, J = 8.8 Hz, 2.4 Hz, 1H), 6.88 (d, J = 9.2 Hz, 1H), 6.19 (d, J = 2.0 Hz, 1H), 6.08 (q, J = 6.4 Hz, 1H), 4.12 (d, J = 8.0 Hz, 2H), 3.82 (d, J = 6.8 Hz, 2H), 3.80 (s, 3H), 2.98 (s, 2H), 1.77 (d, J = 6.4 Hz, 3H) |
| Example 307. | 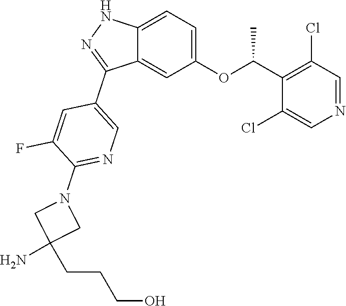 [SMILES: C[C@@H](Oc1ccc2[nH]nc(-c3cnc(N4CC(N)(CCCO)C4)c(F)c3)c2c1)c1c(Cl)cncc1Cl] | (R)-3-(3-amino-1- (5-(5-(1-(3,5- dichloropyridin-4- yl)ethoxy)-1H- indazol-3-yl)-3- fluoropyridin-2- yl)azetidin-3- yl)propan-1-ol | LCMS m/z = 531.2 (M + H); 1HNMR (400 MHz, DMSO-d6) δ 13.10 (s, 1H), 8.57 (s, 2H), 8.36 (d, J = 1.6 Hz, 1H), 7.68 (dd, J = 13.2 Hz, 1.6 Hz, 1H), 7.47 (d, J = 8.8 Hz, 1H), 7.19 (d, J = 1.6 Hz, 1H), 7.10 (dd, J = 8.8 Hz, 2.0 Hz, 1H), 6.14 (q, J = 6.8 Hz, 1H), 3.96 (d, J = 6.8 Hz, 2H), 3.85 (d, J = 7.6 Hz, 2H), 3.45 (t, J = 6.4 Hz, 2H), 1.76 (d, J = 6.8 Hz, 3H), 1.73- 1.72 (m, 2H), 1.60-1.53 (m, 2H) |
| Example 308. | 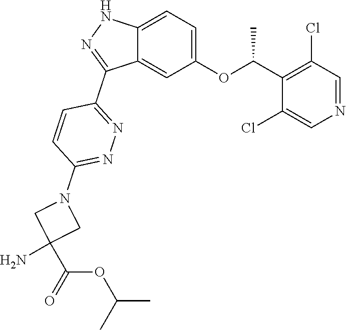 [SMILES: CC(C)OC(=O)C1(N)CN(c2ccc(-c3n[nH]c4ccc(O[C@H](C)c5c(Cl)cncc5Cl)cc34)nn2)C1] | (R)-isopropyl 3- amino-1-(6-(5-(1- (3,5-dichloropyridin- 4-yl)ethoxy)-1H- indazol-3- yl)pyridazin-3- yl)azetidine-3- carboxylate | LCMS m/z = 542.3 (M + H); 1HNMR (400 MHz, DMSO-d6) δ 13.22 (s, 1H), 8.56 (d, J = 5.2 Hz, 2H), 8.01 (d, J = 9.2 Hz, 1H), 7.94 (d, J = 2.0 Hz, 1H), 7.48 (d, J = 9.2 Hz, 1H), 7.10 (dd, J = 9.2 Hz, 2.4 Hz, 1H), 6.99 (d, J = 9.2 Hz, 1H), 6.08 (q, J =6.4 Hz, 1H), 5.06-5.00 (m, 1H), 4.43 (d, J = 8.8 Hz, 2H), 4.06 (d, J = 8.8 Hz, 2H), 1.77 (d, J = 6.4 Hz, 3H), 1.25 (d, J = 6.4 Hz, 6H) |
| Example 309. | 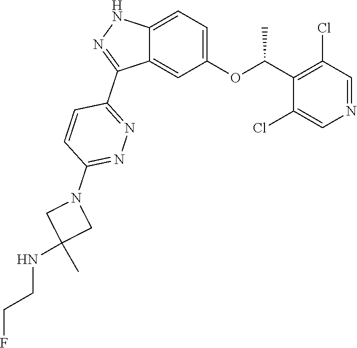 [SMILES: C[C@@H](Oc1ccc2[nH]nc(-c3ccc(N4CC(C)(NCCF)C4)nn3)c2c1)c1c(Cl)cncc1Cl] | (R)-1-(6-(5-(1-(3,5- dichloropyridin-4- yl)ethoxy)-1H- indazol-3- yl)pyridazin-3-yl)- N-(2-fluoroethyl)-3- methylazetidin-3- amine | LCMS m/z = 516.2 (M + H); 1HNMR (400 MHz, DMSO-d6) δ 13.21 (s, 1H), 8.56 (s, 2H), 8.00 (d, J = 9.6 Hz, 1H), 7.94 (d, J = 2.0 Hz, 1H), 7.47 (d, J = 9.2 Hz, 1H), 7.10 (J = 8.8 Hz, 2.0 Hz, 1H), 6.93 (d, J = 9.2 Hz, 1H), 6.08 (q, J = 6.4 Hz, 1H), 4.73-4.47 (m, 2H), 4.15-4.09 (m, 2H), 3.91-3.87 (m, 2H), 3.17-3.03 (m, 2H), 1.77 (d, J = 6.8 Hz, 3H), 1.55 (s, 3H) |
| Example 310. | 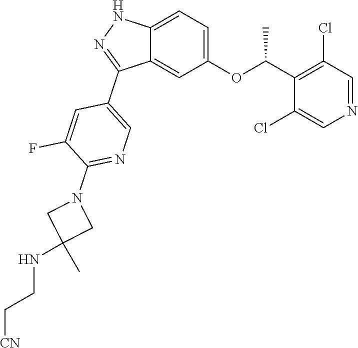 [SMILES: C[C@@H](Oc1ccc2[nH]nc(-c3cnc(N4CC(C)(NCCC#N)C4)c(F)c3)c2c1)c1c(Cl)cncc1Cl] | (R)-3-((1-(5-(5-(1- (3,5-dichloropyridin- 4-yl)ethoxy)-1H- indazol-3-yl)-3- fluoropyridin-2-yl)- 3-methylazetidin-3- yl)amino)propane- nitrile | LCMS m/z = 540.2 (M + H); 1HNMR (400 MHz, DMSO-d6) δ 13.11 (s, 1H), 8.57 (s, 2H), 8.37 (s, 1H), 7.72 (dd, J = 13.2 Hz, 1.2 Hz, 1H), 7.48 (d, J = 9.2 Hz, 1H), 7.20 (s, 1H), 7.11 (dd, J = 9.2 Hz, 2.0 Hz, 1H), 6.17 (q, J = 6.4 Hz, 1H), 4.00 (d, J = 8.0 Hz, 2H), 3.90 (d, J = 6.8 Hz, 2H), 2.81 (t, J = 6.0 Hz, 2H), 2.60 (t, J = 6.0 Hz, 2H), 1.77 (d, J = 6.4 Hz, 3H), 1.45 (s, 3H) |
| Example 311. | 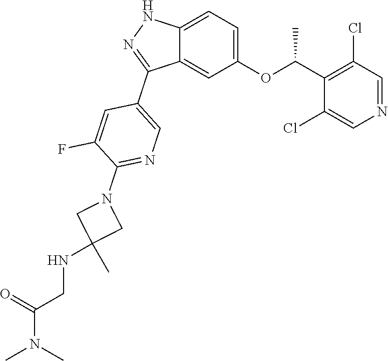 [SMILES: C[C@@H](Oc1ccc2[nH]nc(-c3cnc(N4CC(C)(NCC(=O)N(C)C)C4)c(F)c3)c2c1)c1c(Cl)cncc1Cl] | (R)-2-((1-(5-(5-(1- (3,5-dichloropyridin- 4-yl)ethoxy)-1H- indazol-3-yl)-3- fluoropyridin-2-yl)- 3-methylazetidin-3- yl)amino)-N,N- dimethylacetamide | LCMS m/z = 572.3 (M + H); 1HNMR (400 MHz, DMSO-d6) δ 13.13 (s, 1H), 8.57 (s, 2H), 8.36 (t, J = 1.6 Hz, 1H), 7.69 (dd, J = 13.2 Hz, 2.0 Hz, 1H), 7.47 (d, J = 8.8 Hz, 1H), 7.19 (d, J = 1.6 Hz, 1H), 7.06 (dd, J = 9.2 Hz, 2.0 Hz, 1H), 6.13 (q, J = 6.8 Hz, 1H), 3.99 (d, J = 8.0 Hz, 2H), 3.87 (dd, J = 8.4 Hz, 2.0 Hz, 2H), 3.44 (s, 2H), 2.95 (s, 3H), 2.86 (s, 3H), 1.75 (d, J = 6.8 Hz, 3H), 1.45 (s, 3H) |
| Example 312. | 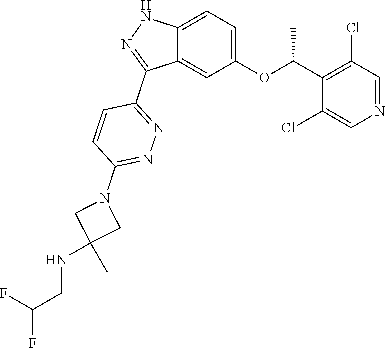 [SMILES: C[C@@H](Oc1ccc2[nH]nc(-c3ccc(N4CC(C)(NCC(F)F)C4)nn3)c2c1)c1c(Cl)cncc1Cl] | (R)-1-(6-(5-(1-(3,5- dichloropyridin-4- yl)ethoxy)-1H- indazol-3- yl)pyridazin-3-yl)- N-(2,2- difluoroethyl)-3- methylazetidin-3- amine | LCMS m/z = 534.2 (M + H); 1HNMR (400 MHz, DMSO-d6) δ 13.17 (s, 1H), 8.56 (s, 2H), 7.98-7.95 (m, 2H), 7.47 (d, J = 9.2 Hz, 1H), 7.10 (dd, J = 8.8 Hz, 2.4 Hz, 1H), 6.88 (d, J = 9.2 Hz, 1H), 6.15-5.85 (m, 2H), 3.96 (d, J = 8.0 Hz, 2H), 3.87 (d, J = 8.4 Hz, 2H), 2.97 (t, J = 15.6 Hz, 2H), 2.81 (brs, 1H), 1.77 (d, J = 6.8 Hz, 3H), 1.46 (s, 3H) |
| Example 313. | 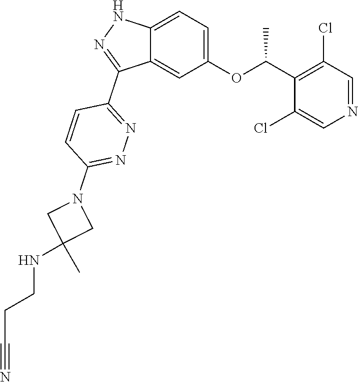 [SMILES: C[C@@H](Oc1ccc2[nH]nc(-c3ccc(N4CC(C)(NCCC#N)C4)nn3)c2c1)c1c(Cl)cncc1Cl] | (R)-3-((1-(6-(5-(1- (3,5-dichloropyridin- 4-yl)ethoxy)-1H- indazol-3- yl)pyridazin-3-yl)-3- methylazetidin-3- yl)amino)propane- nitrile | LCMS m/z = 523.3 (M + H); 1HNMR (400 MHz, DMSO-d6) δ 13.18 (s, 1H), 8.56 (s, 2H), 7.98 (d, J = 9.2 Hz, 1H), 7.94 (d, J = 2.0 Hz, 1H), 7.47 (d, J = 9.2 Hz, 1H), 7.09 (dd, J = 8.8 Hz, 2.0 Hz, 1H), 6.90 (d, J = 9.6 Hz, 1H), 6.08 (q, J = 6.4 Hz, 1H), 3.99 (d, J = 8.0 Hz, 2H), 3.90 (d, J = 8.0 Hz, 2H), 2.95- 2.78 (m, 2H), 2.68-2.60 (m, 2H), 1.77 (d, J = 6.8 Hz, 3H), 1.48 (s, 3H) |
| Example 314. | 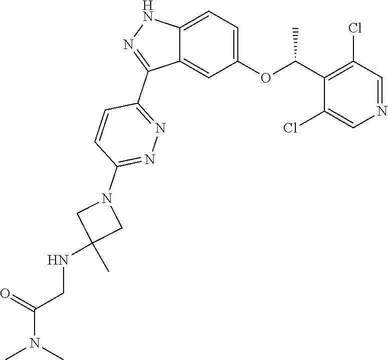 [SMILES: C[C@@H](Oc1ccc2[nH]nc(-c3ccc(N4CC(C)(NCC(=O)N(C)C)C4)nn3)c2c1)c1c(Cl)cncc1Cl] | (R)-2-((1-(6-(5-(1- (3,5-dichloropyridin- 4-yl)ethoxy)-1H- indazol-3- yl)pyridazin-3-yl)-3- methylazetidin-3- yl)amino)-N,N- dimethylacetamide | LCMS m/z = 555.3 (M + H); 1HNMR (400 MHz, DMSO-d6) δ 13.17 (s, 1H), 8.56 (s, 2H), 7.98-7.94 (m, 2H), 7.47 (d, J = 8.8 Hz, 1H), 7.09 (dd, J = 9.2 Hz, 2.4 Hz, 1H), 6.87 (d, J = 9.2 Hz, 1H), 6.08 (q, J = 6.4 Hz, 1H), 3.96 (d, J = 8.4 Hz, 2H), 3.87 (d, J = 8.4 Hz, 2H), 3.46 (s, 2H), 2.96 (s, 3H), 2.86 (s, 3H), 1.76 (d, J = 6.8 Hz, 3H), 1.47 (s, 3H) |
| Example 315. | 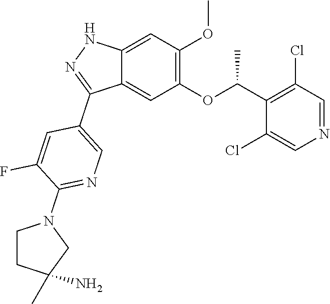 [SMILES: COc1cc2[nH]nc(-c3cnc(N4CC[C@@](C)(N)C4)c(F)c3)c2cc1O[C@H](C)c1c(Cl)cncc1Cl] | (R)-1-(5-(5-((R)-1- (3,5-dichloropyridin- 4-yl)ethoxy)-6- methoxy-1H- indazol-3-yl)-3- fluoropyridin-2-yl)- 3-methylpyrrolidin- 3-amine | LCMS m/z = 531.2 (M + H); 1HNMR (400 MHz, DMSO-d6) δ 12.87 (brs, 1H), 8.59 (s, 2H), 8.27 (s, 1H), 7.60 (dd, J = 14.8 Hz, 1.6 Hz, 1H), 7.08 (s, 1H), 6.99 (s, 1H), 5.98 (q, J = 6.8 Hz, 1H), 3.86 (s, 3H), 3.82-3.68 (m, 2H), 3.49-3.46 (m, 2H), 1.84-1.80 (m, 2H), 1.76 (d, J = 6.8 Hz, 3H), 1.28 (s, 3H) |
| Example 316. | 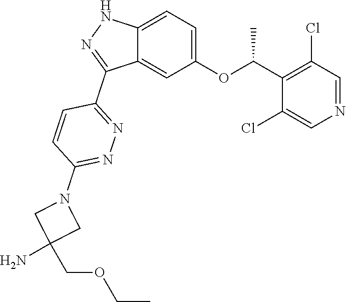 [SMILES: CCOCC1(N)CN(c2ccc(-c3n[nH]c4ccc(O[C@H](C)c5c(Cl)cncc5Cl)cc34)nn2)C1] | (R)-1-(6-(5-(1-(3,5- dichloropyridin-4- yl)ethoxy)-1H- indazol-3- yl)pyridazin-3-yl)-3- (ethoxymethyl)azeti- din-3-amine | LCMS m/z = 514.2 (M + H); 1HNMR (400 MHz, DMSO-d6) δ 13.18 (s, 1H), 8.56 (s, 2H), 7.97 (d, J = 9.2 Hz, 1H), 7.94 (d, J = 2.4 Hz, 1H), 7.47 (d, J = 8.8 Hz, 1H), 7.09 (dd, J = 9.2 Hz, 2.8 Hz, 1H), 6.91 (d, J = 9.6 Hz, 1H), 6.08 (q, J = 6.4 Hz, 1H), 4.07 (d, J = 8.4 Hz, 2H), 3.87 (d, J = 8.8 Hz, 2H), 3.61- 3.52 (m, 4H), 1.77 (d, J = 6.8 Hz, 3H), 1.16 (t, J = 7.2 Hz, 3H) |
| Example 317. | 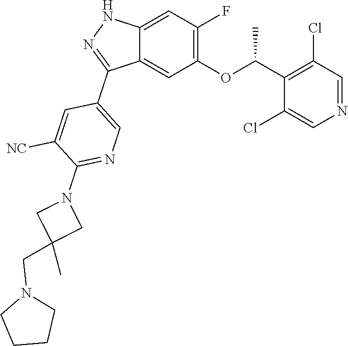 [SMILES: C[C@@H](Oc1cc2c(-c3cnc(N4CC(C)(CN5CCCC5)C4)c(C#N)c3)n[nH]c2cc1F)c1c(Cl)cncc1Cl] | (R)-5-(5-(1-(3,5- dichloropyridin-4- yl)ethoxy)-6-fluoro- 1H-indazol-3-yl)-2- (3-methyl-3- (pyrrolidin-1- ylmethyl)azetidin-1- yl)nicotinonitrile | LCMS m/z= 580.3 (M + H); 1HNMR (400 MHz, DMSO-d6) δ 13.24 (brs, 1H), 8.73 (d, J = 2.8 Hz, 1H), 8.60 (s, 2H), 8.12 (d, J = 2.8 Hz, 1H), 7.47 (d, J = 10.8 Hz, 1H), 7.23 (d, J = 8.0 Hz, 1H), 6.17 (q, J = 6.4 Hz, 1H), 4.10 (d, J = 8.4 Hz, 2H), 3.98 (d, J = 8.8 Hz, 2H), 2.78- 2.52 (m, 6H), 1.82 (d, J = 6.8 Hz, 3H), 1.77-1.64 (m, 4H), 1.39 (s, 3H) |
| Example 318. | 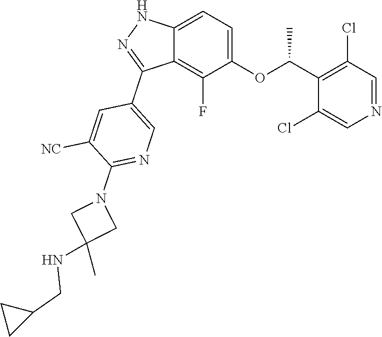 [SMILES: C[C@@H](Oc1ccc2[nH]nc(-c3cnc(N4CC(C)(NCC5CC5)C4)c(C#N)c3)c2c1F)c1c(Cl)cncc1Cl] | (R)-2-(3-((cyclo- propylmethyl) amino)-3- methylazetidin-1- yl)-5-(5-(1-(3,5- dichloropyridin-4- yl)ethoxy)-4-fluoro- 1H-indazol-3- yl)nicotinonitrile | LCMS m/z = 566.2 (M + H); 1HNMR (400 MHz, DMSO-d6) δ 13.50 (s, 1H), 8.69 (s, 1H), 8.61 (s, 2H), 8.21 (s, 1H), 7.28 (d, J = 9.2 Hz, 1H), 7.15-7.11 (m, 1H), 5.99 (q, J = 6.8 Hz, 1H), 4.20-4.14 (m, 2H), 4.05- 4.00 (m, 2H), 2.47-2.39 (m, 2H), 1.79 (d, J = 6.4 Hz, 3H), 1.43 (s, 3H), 0.92-0.81 (m, 1H), 0.46-0.42 (m, 2H), 0.20-0.13 (m, 2H) |
| Example 319. | 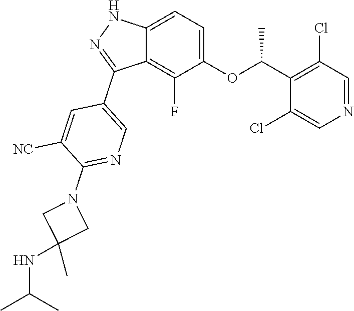 [SMILES: CC(C)NC1(C)CN(c2ncc(-c3n[nH]c4ccc(O[C@H](C)c5c(Cl)cncc5Cl)c(F)c34)cc2C#N)C1] | (R)-5-(5-(1-(3,5- dichloropyridin-4- yl)ethoxy)-4-fluoro- 1H-indazol-3-yl)-2- (3-(isopropylamino)- 3-methylazetidin-1- yl)nicotinonitrile | LCMS m/z = 554.3 (M + H); 1HNMR (400 MHz, DMSO-d6) δ 13.50 (s, 1H), 8.69 (s, 1H), 8.61 (s, 2H), 8.22 (s, 1H), 7.28 (d, J = 8.8 Hz, 1H), 7.15-7.11 (m, 1H), 6.00 (q, J = 6.8 Hz, 1H), 4.19-4.02 (m, 4H), 3.10- 2.90 (m, 1H), 1.79 (d, J = 6.8 Hz, 3H), 1.48 (s, 3H), 1.11-0.93 (m, 6H) |
| Example 320. | 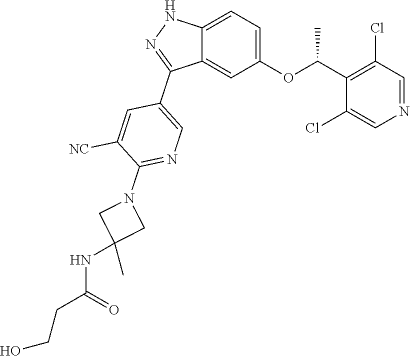 [SMILES: C[C@@H](Oc1ccc2[nH]nc(-c3cnc(N4CC(C)(NC(=O)CCO)C4)c(C#N)c3)c2c1)c1c(Cl)cncc1Cl] | (R)-1-(3-cyano-5-(5- (1-(3,5- dichloropyridin-4- yl)ethoxy)-1H- indazol-3-yl)pyridin- 2-yl)-N-(2- hydroxyethyl)-3- methylazetidine-3- carboxamide | LCMS m/z = 566.3 (M + H); 1HNMR (400 MHz, DMSO-d6) δ 13.18 (s, 1H), 8.77 (d, J = 2.4 Hz, 1H), 8.57 (s, 2H), 8.15 (d, J = 2.4 Hz, 1H), 8.04 (t, J = 6.0 Hz, 1H), 7.50 (d, J = 9.2 Hz, 1H), 7.17 (d, J = 1.6 Hz, 1H), 7.12 (dd, J = 8.8 Hz, 2.0 Hz, 1H), 6.17 (q, J = 6.8 Hz, 1H), 4.70 (t, J = 5.2 Hz, 1H), 4.52 (d, J = 8.8 Hz, 2H), 4.06 (d, J = 8.8 Hz, 2H), 3.47 (q, J = 6.4 Hz, 2H), 3.20 (q, J = 5.6 Hz, 2H), 1.77 (d, J = 6.8 Hz, 3H), 1.56 (s, 3H) |
| Example 321. | 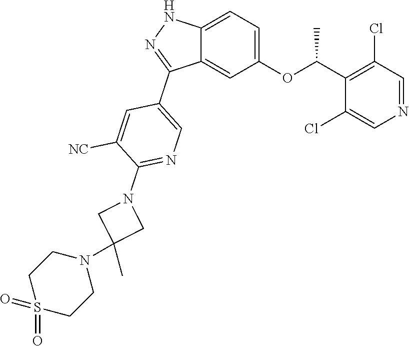 [SMILES: C[C@@H](Oc1ccc2[nH]nc(-c3cnc(N4CC(C)(N5CCS(=O)(=O)CC5)C4)c(C#N)c3)c2c1)c1c(Cl)cncc1Cl] | (R)-5-(5-(1-(3,5- dichloropyridin-4- yl)ethoxy)-1H- indazol-3-yl)-2-(3- (1,1- dioxidothio- morpholino)-3- methylazetidin-1- yl)nicotinonitrile | LCMS m/z = 612.1 (M + H); 1HNMR (400 MHz, DMSO-d6) δ 13.18 (s, 1H), 8.77 (d, J = 1.2 Hz, 1H), 8.58 (s, 2H), 8.17 (d, J = 1.6 Hz, 1H), 7.50 (d, J = 9.2 Hz, 1H), 7.16 (s, 1H), 7.12 (d, J = 10.4 Hz, 1H), 6.17 (q, J = 6.8 Hz, 1H), 4.15 (d, J = 8.8 Hz, 2H), 4.05 (d, J = 8.4 Hz, 2H), 3.18-3.10 (m, 4H), 2.90-2.85 (m, 4H), 1.77 (d, J = 6.8 Hz, 3H), 1.38 (s, 3H) |
| Example 322. | 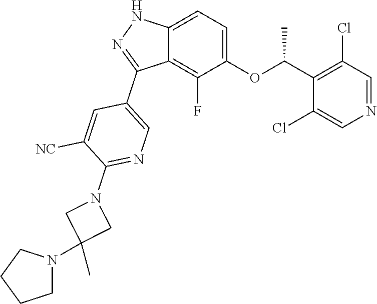 [SMILES: C[C@@H](Oc1ccc2[nH]nc(-c3cnc(N4CC(C)(N5CCCC5)C4)c(C#N)c3)c2c1F)c1c(Cl)cncc1Cl] | (R)-5-(5-(1-(3,5- dichloropyridin-4- yl)ethoxy)-4-fluoro- 1H-indazol-3-yl)-2- (3-methyl-3- (pyrrolidin-1- yl)azetidin-1- yl)nicotinonitrile | LCMS m/z = 566.2 (M + H); 1HNMR (400 MHz, DMSO-d6) δ 13.51 (s, 1H), 8.69 (t, J = 1.6 Hz, 1H), 8.61 (s, 2H), 8.22 (s, 1H), 7.28 (d, J = 8.8 Hz, 1H), 7.13 (t, J = 8.4 Hz, 1H), 6.00 (q, J = 6.8 Hz, 1H), 4.31 (d, J = 8.8 Hz, 2H), 3.99 (d, J = 8.8 Hz, 2H), 2.67-2.65 (m, 4H), 1.79 (d, J = 6.8 Hz, 3H), 1.77-1.68 (m, 4H), 1.45 (s, 3H) |
| Example 323. | 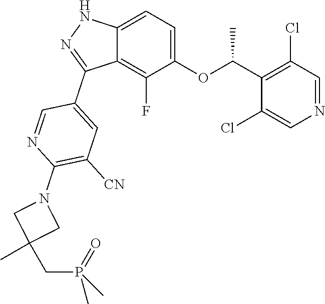 [SMILES: C[C@@H](Oc1ccc2[nH]nc(-c3cnc(N4CC(C)(CP(C)(C)=O)C4)c(C#N)c3)c2c1F)c1c(Cl)cncc1Cl] | (R)-5-(5-(1-(3,5- dichloropyridin-4- yl)ethoxy)-4-fluoro- 1H-indazol-3-yl)-2- (3-((dimethyl- phosphoryl) methyl)-3- methylazetidin-1- yl)nicotinonitrile | LCMS m/z = 587.2 (M + H); 1HNMR (400 MHz, DMSO-d6) δ 13.49 (brs, 1H), 8.69 (t, J = 2.0 Hz, 1H), 8.61 (s, 2H), 8.21 (t, J = 2.0 Hz, 1H), 7.29 (d, J = 8.8 Hz, 1H), 7.14 (t, J = 7.6 Hz, 1H), 6.00 (q, J = 6.4 Hz, 1H), 4.28 (d, J = 8.8 Hz, 2H), 4.04 (d, J = 8.8 Hz, 2H), 2.24 (d, J = 11.2 Hz, 2H), 1.80 (d, J = 6.4 Hz, 3H), 1.55 (s, 3H), 1.46 (s, 3H), 1.43 (s, 3H) |
| Example 324. | 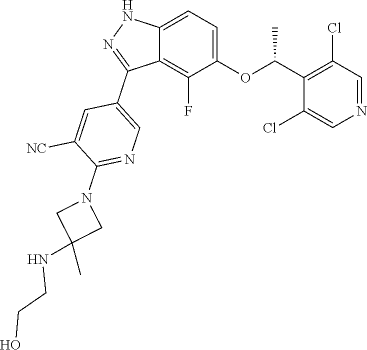 [SMILES: C[C@@H](Oc1ccc2[nH]nc(-c3cnc(N4CC(C)(NCCO)C4)c(C#N)c3)c2c1F)c1c(Cl)cncc1Cl] | (R)-5-(5-(1-(3,5- dichloropyridin-4- yl)ethoxy)-4-fluoro- 1H-indazol-3-yl)-2- (3-((2-hydroxy- ethyl)amino)- 3-methylazetidin- 1-yl)nicotinonitrile | LCMS m/z = 556.2 (M + H); 1HNMR (400 MHz, DMSO-d6) δ 13.50 (brs, 1H), 8.69 (s, 1H), 8.61 (s, 2H), 8.21 (s, 1H), 7.28 (d, J = 9.2 Hz, 1H), 7.12 (t, J = 8.0 Hz, 1H), 6.00 (q, J = 6.8 Hz, 1H), 4.52 (t, J = 5.6 Hz, 1H), 4.11 (d, J = 8.8 Hz, 2H), 4.04 (d, J = 8.8 Hz, 2H), 3.46 (q, J = 6.0 Hz, 2H), 2.61 (t, J = 6.0 Hz, 2H), 1.79 (d, J = 6.4 Hz, 3H), 1.42 (s, 3H) |
| Example 325. | 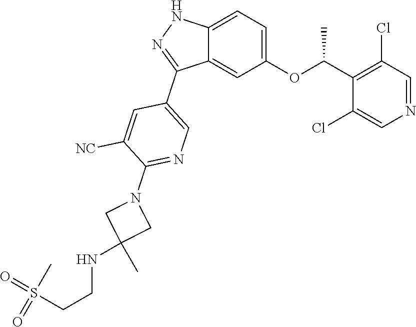 [SMILES: C[C@@H](Oc1ccc2[nH]nc(-c3cnc(N4CC(C)(NCCS(C)(=O)=O)C4)c(C#N)c3)c2c1)c1c(Cl)cncc1Cl] | 5-[5-[(1R)-1-(3,5- dichloro-4- pyridyl)ethoxy]-1H- indazol-3-yl]-2-[3- methyl-3-(2- methylsulfonylethyl- amino)azetidin-1- yl]pyridine-3- carbonitrile | LCMS m/z = 600.0 (M + H); 1HNMR (400 MHz, DMSO-d6) δ 13.16 (s, 1H), 8.78 (d, J = 2.3 Hz, 1H), 8.58 (s, 2H), 8.15 (d, J = 2.3 Hz, 1H), 7.49 (d, J = 9.0 Hz, 1H), 7.19 - 7.15 (m, 1H), 7.11 (dd, J = 2.3, 9.0 Hz, 1H), 6.15 (q, J = 6.6 Hz, 1H), 4.15 (d, J = 8.7 Hz, 2H), 4.08 (d, J = 8.7 Hz, 2H), 3.23 (t, J = 6.7 Hz, 2H), 3.05 (s, 3H), 2.97 (br s, 2H), 2.76 - 2.60 (m, 1H), 1.77 (d, J = 6.7 Hz, 3H), 1.46 (s, 3H) |
| Example 326. | 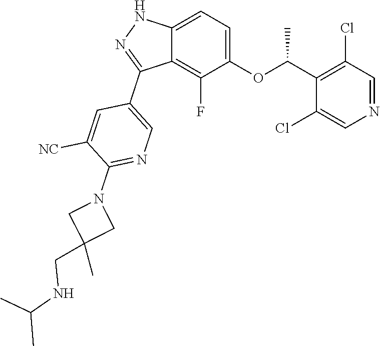 [SMILES: CC(C)NCC1(C)CN(c2ncc(-c3n[nH]c4ccc(O[C@H](C)c5c(Cl)cncc5Cl)c(F)c34)cc2C#N)C1] | (R)-5-(5-(1-(3,5- dichloropyridin-4- yl)ethoxy)-4-fluoro- 1H-indazol-3-yl)-2- (3- ((isopropylamino) methyl)-3- methylazetidin-1- yl)nicotinonitrile | LCMS m/z = 568.2 (M + H); 1HNMR (400 MHz, DMSO-d6) δ 13.49 (brs, 1H), 8.67 (t, J = 2.0 Hz, 1H), 8.61 (s, 2H), 8.19 (t, J = 2.0 Hz, 1H), 7.29 (d, J = 9.2 Hz, 1H), 7.14-7.09 (m, 1H), 6.00 (q, J = 6.8 Hz, 1H), 4.11 (d, J = 8.8 Hz, 2H), 3.87 (d, J = 8.8 Hz, 2H), 2.74-2.66 (m, 3H), 1.80 (d, J = 6.8 Hz, 3H), 1.30 (s, 3H), 1.02 (d, J = 6.0 Hz, 6H) |
| Example 327. | 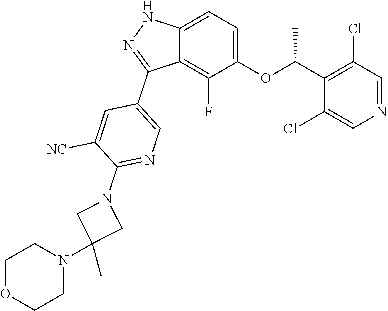 [SMILES: C[C@@H](Oc1ccc2[nH]nc(-c3cnc(N4CC(C)(N5CCOCC5)C4)c(C#N)c3)c2c1F)c1c(Cl)cncc1Cl] | (R)-5-(5-(1-(3,5- dichloropyridin-4- yl)ethoxy)-4-fluoro- 1H-indazol-3-yl)-2- (3-methyl-3- morpholinoazetidin- 1-yl)nicotinonitrile | LCMS m/z = 582.2 (M + H); 1HNMR (400 MHz, DMSO-d6) δ 13.50 (s, 1H), 8.69 (t, J = 2.0 Hz, 1 H), 8.60 (s, 2H), 8.23- 8.22 (m, 1H), 7.28 (d, J = 9.2 Hz, 1H), 7.14-7.10 (m, 1H), 5.99 (q, J = 6.8 Hz, 1H), 4.14- 4.10 (m, 2H), 4.00-3.96 (m, 2H), 3.60 (t, J = 4.0 Hz, 4H), 2.49 (t, J = 2.0 Hz, 4H), 1.79 (d, J = 6.8 Hz, 3H), 1.34 (s, 3H) |
| Example 328. | 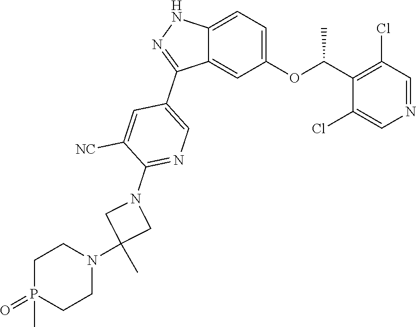 [SMILES: C[C@@H](Oc1ccc2[nH]nc(-c3cnc(N4CC(C)(N5CCP(C)(=O)CC5)C4)c(C#N)c3)c2c1)c1c(Cl)cncc1Cl] | (R)-5-(5-(1-(3,5- dichloropyridin-4- yl)ethoxy)-1H- indazol-3-yl)-2-(3- methyl-3-(4-methyl- 4-oxido-1,4- azaphosphinan-1- yl)azetidin-1 -yl)nicotinonitrile | LCMS m/z = 610.2 (M + H); 1HNMR (400 MHz, DMSO-d6) δ 13.18 (s, 1H), 8.77 (d, J = 2.0 Hz, 1H), 8.58 (s, 2H), 8.16 (d, J = 2.0 Hz, 1H), 7.50 (d, J = 8.8 Hz, 1H), 7.16 (s, 1H), 7.12 (dd, J = 9.2 Hz, 2.0 Hz, 1H), 6.15 (q, J = 6.4 Hz, 1H), 4.08 (d, J = 8.0 Hz, 2H), 4.03 (d, J = 8.0 Hz, 2H), 2.81-2.72 (m, 2H), 2.61- 2.54 (m, 2H), 1.88-1.75 (m, 7H), 1.45 (d, J = 12.8 Hz, 3H), 1.35 (s, 3H) |
| Example 329. | 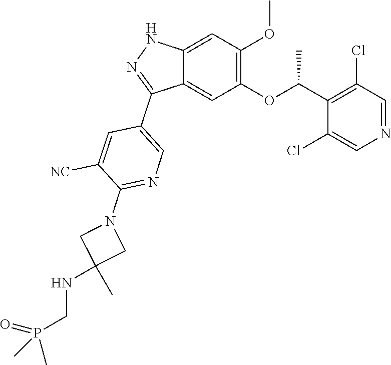 [SMILES: COc1cc2[nH]nc(-c3cnc(N4CC(C)(NCP(C)(C)=O)C4)c(C#N)c3)c2cc1O[C@H](C)c1c(Cl)cncc1Cl] | 5-[5-[(1R)-1-(3,5- dichloro-4- pyridyl)ethoxy]-6- methoxy-1H- indazol-3-yl]-2-[3- (dimethyl- phosphoryl- methylamino)-3- methyl-azetidin-1- yl]pyridine-3- carbonitrile | LCMS m/z = 614.2 (M + H); 1HNMR (400 MHz, DMSO-d6) δ 12.96 (br s, 1H), 8.72 (d, J = 2.3 Hz, 1H), 8.59 (s, 2H), 8.06 (d, J = 2.3 Hz, 1H), 7.04 (s, 1H), 7.01 (s, 1H), 6.03-5.96 (m, 1H), 4.17 (d, J = 8.9 Hz, 2H), 4.03 (d, J = 8.7 Hz, 2H), 3.86 (s, 3H), 2.85 (t, J = 8.7 Hz, 2H), 2.68-2.52 (m, 1H), 1.76 (d, J = 6.6 Hz, 3H), 1.45 (s, 3H), 1.42 (s, 3H), 1.39 (s, 3H) |
| Example 330. | 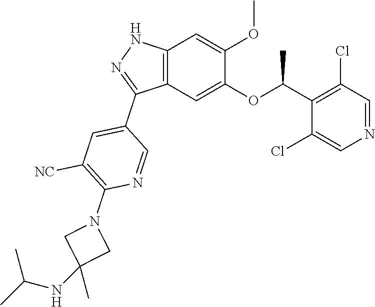 [SMILES: COc1cc2[nH]nc(-c3cnc(N4CC(C)(NC(C)C)C4)c(C#N)c3)c2cc1O[C@@H](C)c1c(Cl)cncc1Cl] | (S)-5-(5-(1-(3,5- dichloropyridin-4- yl)ethoxy)-6- methoxy-1H- indazol-3-yl)-2-(3- (isopropylamino)-3- methylazetidin-1- yl)nicotinonitrile | LCMS m/z = 566.3 (M + H); 1HNMR (400 MHz, DMSO-d6) δ 12.96 (s, 1H), 8.72 (d, J = 2.4 Hz, 1H), 8.59 (s, 2H), 8.06 (d, J = 2.4 Hz, 1H), 7.04 (s, 1H), 7.01 (s, 1H), 6.00 (q, J = 7.2 Hz, 1H), 4.15-4.09 (m, 4H), 3.86 (s, 3H), 3.01-2.93 (m, 1H), 1.76 (d, J = 6.8 Hz, 3H), 1.47 (s, 3H), 1.00 (d, J = 6.0 Hz, 6H) |
| Example 331. | 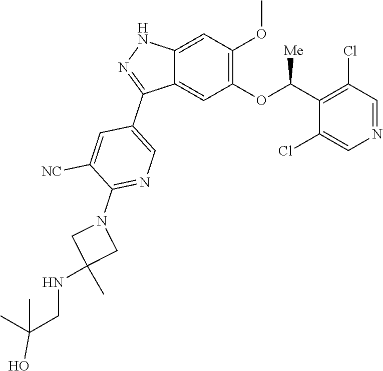 [SMILES: COc1cc2[nH]nc(-c3cnc(N4CC(C)(NCC(C)(C)O)C4)c(C#N)c3)c2cc1O[C@@H](C)c1c(Cl)cncc1Cl] | (S)-5-(5-(1-(3,5- dichloropyridin-4- yl)ethoxy)-6- methoxy-1H- indazol-3-yl)-2-(3- ((2-hydroxy-2- methylpro- pyl)amino)- 3-methylazetidin- 1-yl)nicotinonitrile | LCMS m/z = 596.3 (M + H); 1HNMR (400 MHz, DMSO-d6) δ 12.97 (s, 1H), 8.72 (d, J = 2.4 Hz, 1H), 8.59 (s, 2H), 8.06 (d, J = 2.0 Hz, 1H), 7.03 (s, 1H), 7.01 (s, 1H), 6.00 (q, J = 6.8 Hz, 1H), 4.24 (s, 1H), 4.13 (d, J = 8.4 Hz, 2H), 4.04 (d, J = 8.8 Hz, 2H), 3.86 (s, 3H), 2.43 (s, 2H), 1.76 (d, J = 6.8 Hz, 3H), 1.43 (s, 3H), 1.12 (s, 6H) |
| Example 332. | 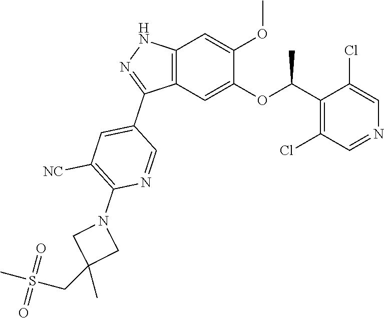 [SMILES: COc1cc2[nH]nc(-c3cnc(N4CC(C)(CS(C)(=O)=O)C4)c(C#N)c3)c2cc1O[C@@H](C)c1c(Cl)cncc1Cl] | (S)-5-(5-(1-(3,5- dichloropyridin-4- yl)ethoxy)-6- methoxy-1H- indazol-3-yl)-2-(3- methyl-3-((methyl- sulfonyl)meth- yl)azetidin-1- yl)nicotinonitrile | LCMS m/z = 601.2 (M + H); 1HNMR (400 MHz, DMSO-d6) δ 12.98 (s, 1H), 8.73 (d, J = 2.0 Hz, 1H), 8.59 (s, 2H), 8.09 (d, J = 2.4 Hz, 1H), 7.04 (s, 1H), 7.01 (s, 1H), 6.02 (q, J = 6.4 Hz, 1H), 4.38 (d, J = 8.8 Hz, 2H), 4.06 (d, J = 8.4 Hz, 2H), 3.86 (s, 3H), 3.71 (s, 2H), 3.04 (s, 3H), 1.77 (d, J = 6.4 Hz, 3H), 1.61 (s, 3H) |
| Example 333. | 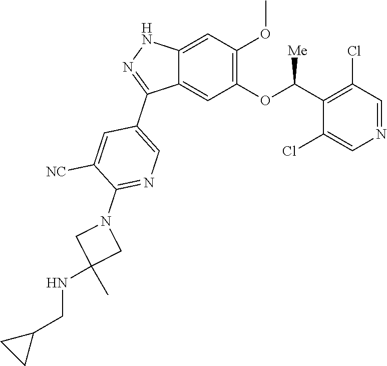 [SMILES: COc1cc2[nH]nc(-c3cnc(N4CC(C)(NCC5CC5)C4)c(C#N)c3)c2cc1O[C@@H](C)c1c(Cl)cncc1Cl] | (S)-2-(3- ((cyclopropyl- methyl) amino)-3- methylazetidin-1- yl)-5-(5-(1-(3,5- dichloropyridin-4- yl)ethoxy)-6- methoxy-1H- indazol-3- yl)nicotinonitrile | LCMS m/z = 578.2 (M + H); 1HNMR (400 MHz, DMSO-d6) δ 12.96 (s, 1H), 8.72 (d, J = 2.4 Hz, 1H), 8.59 (s, 2H), 8.06 (d, J = 2.4 Hz, 1H), 7.03 (s, 1H), 7.01 (s, 1H), 6.00 (q, J = 7.2 Hz, 1H), 4.14 (d, J = 8.8 Hz, 2H), 4.03 (d, J = 8.4 Hz, 2H), 3.86 (s, 3H), 2.42 (d, J = 6.8 Hz, 2H), 1.76 (d, J = 6.8 Hz, 3H), 1.43 (s, 3H), 0.89-0.85 (m, 1H), 0.45-0.40 (m, 2H), 0.17- 0.15 (m, 2H) |
| Example 334. | 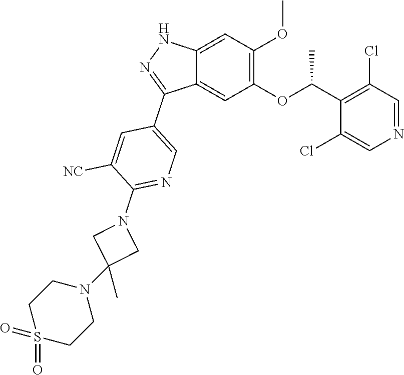 [SMILES: COc1cc2[nH]nc(-c3cnc(N4CC(C)(N5CCS(=O)(=O)CC5)C4)c(C#N)c3)c2cc1O[C@H](C)c1c(Cl)cncc1Cl] | (R)-5-(5-(1-(3,5- dichloropyridin-4- yl)ethoxy)-6- methoxy-1H- indazol-3-yl)-2-(3- (1,1-dioxidothio- morpholino)-3- methylazetidin-1- yl)nicotinonitrile | LCMS m/z = 642.2 (M + H); 1HNMR (400 MHz, DMSO-d6) δ 12.98 (brs, 1H), 8.73 (d, J = 2.4 Hz, 1H), 8.59 (s, 2H), 8.10 (d, J = 2.4 Hz, 1H), 7.04 (s, 1H), 7.01 (s, 1H), 6.03 (q, J = 6.8 Hz, 1H), 4.14 (d, J = 8.4 Hz, 2H), 4.05 (d, J = 8.4 Hz, 2H), 3.86 (s, 3H), 3.17-3.10 (m, 4H), 2.89-2.79 (m, 4H), 1.77 (d, J = 6.8 Hz, 3H), 1.38 (s, 3H) |
| Example 335. | 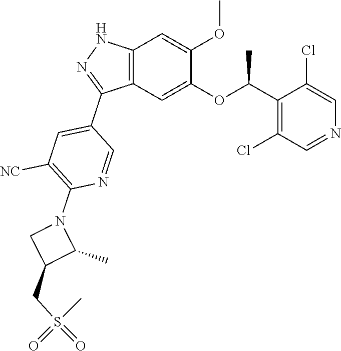 [SMILES: COc1cc2[nH]nc(-c3cnc(N4C[C@H](CS(C)(=O)=O)[C@H]4C)c(C#N)c3)c2cc1O[C@@H](C)c1c(Cl)cncc1Cl] | 5-(5-((S)-1-(3,5- dichloropyridin-4- yl)ethoxy)-6- methoxy-1H- indazol-3-yl)-2- ((2R,3S)-2-methyl- 3-((meth- ylsulfonyl)meth- yl)azetidin-1- yl)nicotinonitrile | LCMS m/z = 601.5 (M + H); 1HNMR (400 MHz, DMSO-d6) δ 12.99 (s, 1H), 8.71 (d, J = 2.0 Hz, 1H), 8.59 (s, 2H), 8.10 (d, J = 2.0 Hz, 1H), 7.02 (s, 1H), 7.01 (s, 1H), 6.02 (q, J = 6.4 Hz, 1H), 4.61 (t, J = 8.0 Hz, 1H), 4.51-4.45(m, 1H), 4.08- 4.04 (m, 1H), 3.86 (s, 3H), 3.65-3.54 (m, 2H), 3.01 (s, 3H), 2.91-2.84 (m, 1H), 1.77 (d, J = 6.4 Hz, 3H), 1.56 (d, J = 6.0 Hz, 3H) |
| Example 336. | 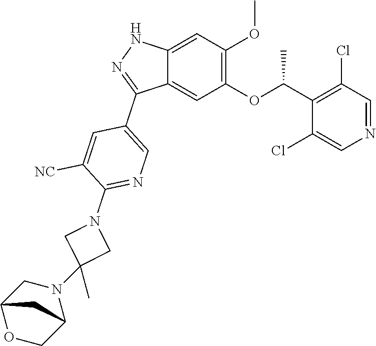 [SMILES: COc1cc2[nH]nc(-c3cnc(N4CC(C)(N5C[C@@H]6C[C@H]5CO6)C4)c(C#N)c3)c2cc1O[C@H](C)c1c(Cl)cncc1Cl] | -(3-((1S,4S)-2-oxa- 5-aza- bicyclo[2.2.1]hep- tan-5-y1)-3- methylazetidin-1- yl)-5-(5-((R)-1-(3,5- dichloropyridin-4- yl)ethoxy)-6- methoxy-1H- indazol-3- yl)nicotinonitrile | LCMS m/z = 606.3 (M + H); 1HNMR (400 MHz, DMSO-d6) δ 12.96 (s, 1H), 8.71 (d, J = 2.0 Hz, 1H), 8.59 (s, 2H), 8.07 (d, J = 2.4 Hz, 1H), 7.05 (s, 1H), 7.01 (s, 1H), 6.00 (q, J = 6.8 Hz, 1H), 4.44 (s, 1H), 4.38 (d, J = 8.8 Hz, 1H), 4.18 (d, J = 8.8 Hz, 1H), 4.10 (d, J = 8.8 Hz, 1H), 4.02 (d, J = 9.2 Hz, 1H), 3.86 (s, 3H), 3.75 (s, 1H), 3.59- 3.55 (m, 2H), 2.92-2.90 (m, 2H), 1.78-1.73 (m, 4H), 1.66 (d, J = 10.8Hz, 1H), 1.52 (s, 3H) |
| Example 337. | 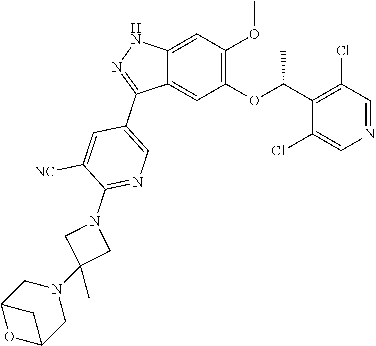 [SMILES: COc1cc2[nH]nc(-c3cnc(N4CC(C)(N5CC6CC(C5)O6)C4)c(C#N)c3)c2cc1O[C@H](C)c1c(Cl)cncc1Cl] | 2-(3-(6-oxa-3-aza- bicyclo[3.1.1]hep- tan-3-yl)-3- methylazetidin-1- yl)-5-(5-((R)-1-(3,5- dichloropyridin-4- yl)ethoxy)-6- methoxy-1H- indazol-3- yl)nicotinonitrile | LCMS m/z= 606.3 (M + H); 1HNMR (400 MHz, DMSO-d6) δ 12.98 (s, 1H), 8.72 (d, J = 2.4 Hz, 1H), 8.59 (s, 2H), 8.08 (d, J = 2.8 Hz, 1H), 7.04 (s, 1H), 7.00 (s, 1H), 5.99 (q, J = 6.8 Hz, 1H), 4.50 (d, J = 6.0 Hz, 2H), 4.33 (d, J = 8.8 Hz, 2H), 4.02 (d, J = 8.8 Hz, 2H), 3.85 (s, 3H), 3.02 (d, J = 11.2 Hz, 2H), 2.87-2.84 (m, 3H), 2.17 (d, J = 8.0 Hz, 1H), 1.75 (d, J = 6.8 Hz, 3H), 1.50 (s, 3H) |
| Example 338. | 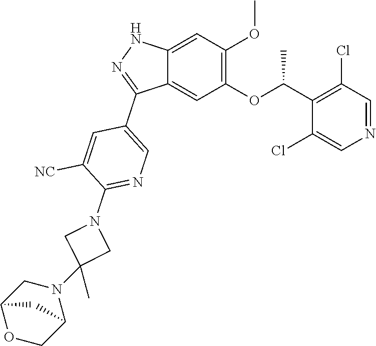 [SMILES: COc1cc2[nH]nc(-c3cnc(N4CC(C)(N5C[C@H]6C[C@@H]5CO6)C4)c(C#N)c3)c2cc1O[C@H](C)c1c(Cl)cncc1Cl] | 2-(3-((1R,4R)-2- oxa-5-aza- bicyclo[2.2.1]hep- tan-5-yl)-3- methylazetidin-1- yl)-5-(5-((R)-1-(3,5- dichloropyridin-4- yl)ethoxy)-6- methoxy-1H- indazol-3- yl)nicotinonitrile | LCMS m/z = 606.3 (M + H); 1HNMR (400 MHz, DMSO-d6) δ 12.95 (s, 1H), 8.71 (d, J = 2.0 Hz, 1H), 8.58 (s, 2H), 8.06 (d, J = 2.8 Hz, 1H), 7.04 (s, 1H), 7.01 (s, 1H), 5.99 (q, J = 6.8 Hz, 1H), 4.43 (s, 1H), 4.38 (d, J = 8.8 Hz, 1H), 4.18 (d, J = 8.8 Hz, 1H), 4.11-4.00 (m, 2H), 3.86 (s, 3H), 3.75 (s, 1H), 3.59- 3.55 (m, 2H), 2.91 (s, 2H), 1.77-1.74 (m, 4H), 1.66 (d, J = 9.6 Hz, 1H), 1.52 (s, 3H) |
| Example 339. | 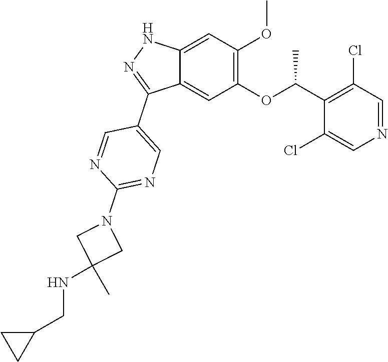 | (R)-N-(cyclopropyl- methyl)-1-(5-(5-(1- (3,5-dichloropyridin- 4-yl)ethoxy)-6- methoxy-1H- indazol-3- yl)pyrimidin-2-yl)- 3-methylazetidin-3- amine | LCMS m/z = 540.3 (M + H); 1HNMR (400 MHz, DMSO-d6) δ 12.91 (s, 1H), 8.69 (s, 2H), 8.59 (s, 2H), 7.14 (s, 1H), 7.00 (s, 1H), 6.01 (q, J = 6.4 Hz, 1H), 3.94 (d, J = 8.4 Hz, 2H), 3.85 (s, 3H), 3.80 (d, J = 8.8 Hz, 2H), 2.40 (d, J = 6.8 Hz, 2H), 1.75 (d, J = 7.2 Hz, 3H), 1.40 (s, 3H), 0.86-0.84 (m, 1H), 0.43-0.39 (m, 2H), 0.16-0.12 (m, 2H) |
| Example 340. | 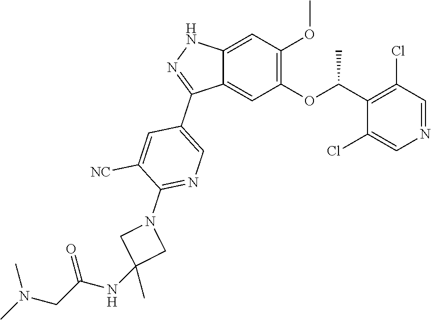 [SMILES: COc1cc2[nH]nc(-c3cnc(N4CC(C)(NC(=O)CN(C)C)C4)c(C#N)c3)c2cc1O[C@H](C)c1c(Cl)cncc1Cl] | (R)-N-(1-(3-cyano- 5-(5-(1-(3,5- dichloropyridin-4- yl)ethoxy)-6- methoxy-1H- indazol-3-yl)pyridin- 2-yl)-3- methylazetidin-3- yl)-2- (dimethylamino)- acetamide | LCMS m/z = 609.3 (M + H); 1HNMR (400 MHz, DMSO-d6) δ 12.96 (s, 1H), 8.72 (d, J = 2.4 Hz, 1H), 8.58 (s, 2H), 8.33 (s, 1H), 8.08 (d, J = 2.0 Hz, 1H), 7.05 (s, 1H), 7.00 (s, 1H), 6.01 (q, J = 3.6 Hz, 1H), 4.42 (d, J = 9.2 Hz, 2H), 4.18 (d, J = 9.2 Hz, 2H), 3.86 (s, 3H), 2.90 (s, 2H), 2.23 (s, 6H), 1.77 (d, J = 6.4 Hz, 3H), 1.58 (s, 3H) |
| Example 341. | 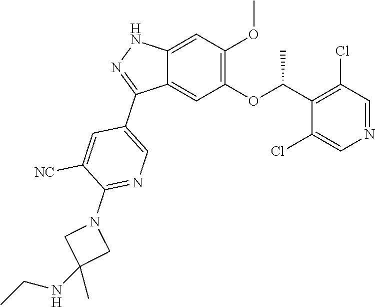 [SMILES: CCNC1(C)CN(c2ncc(-c3n[nH]c4cc(OC)c(O[C@H](C)c5c(Cl)cncc5Cl)cc34)cc2C#N)C1] | (R)-5-(5-(1-(3,5- Dichloropyridin-4- yl)ethoxy)-6- methoxy-1H- indazol-3-yl)-2-(3- (ethylamino)-3- methylazetidin-1- yl)nicotinonitrile HCl | LCMS m/z = 552.2 (M + H); 1HNMR (400 MHz, DMSO-d6) δ 13.07 (s, 1H), 9.75 (br s, 2H), 8.77 (s, 1H), 8.59 (s, 2H), 8.16 (s, 1H), 7.04 (s, 1H), 7.02 (s, 1H), 6.00 (q, J = 6.4 Hz, 1H), 4.55 (br d, J = 9.8 Hz, 2H), 4.25 (br d, J = 9.8 Hz, 2H), 3.86 (s, 3H), 3.03 (br s, 2H), 1.76 (br d, J = 6.4 Hz, 3H), 1.69 (s, 3H), 1.27 (br t, J = 6.9 Hz, 3H |
| Example 342. | 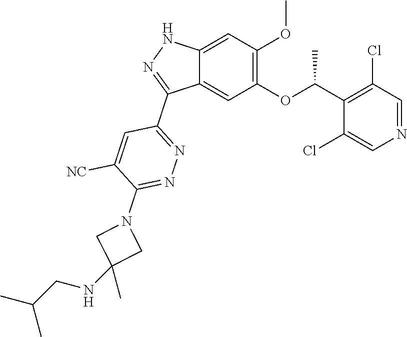 [SMILES: COc1cc2[nH]nc(-c3cc(C#N)c(N4CC(C)(NCC(C)C)C4)nn3)c2cc1O[C@H](C)c1c(Cl)cncc1Cl] | (R)-6-(5-(1-(3,5- dichloropyridin-4- yl)ethoxy)-6- methoxy-1H- indazol-3-yl)-3-(3- (isobutylamino)-3- methylazetidin-1- yl)pyridazine-4- carbonitrile | LCMS m/z = 581.3 (M + H); 1HNMR (400 MHz, DMSO-d6) δ 13.18 (s, 1H), 8.57 (s, 2H), 8.26 (s, 1H), 7.69 (s, 1H), 7.03 (s, 1H), 5.91 (q, J = 6.4 Hz, 1H), 4.23 (d, J = 8.8 Hz, 2H), 4.13 (d, J = 8.8 Hz, 2H), 3.87 (s, 3H), 2.36 (d, J = 6.0 Hz, 2H), 1.75 (d, J = 6.8 Hz, 3H), 1.63-1.60 (m, 1H), 1.45 (s, 3H), 0.92 (d, J = 6.4 Hz, 6H) |
| Example 343. | 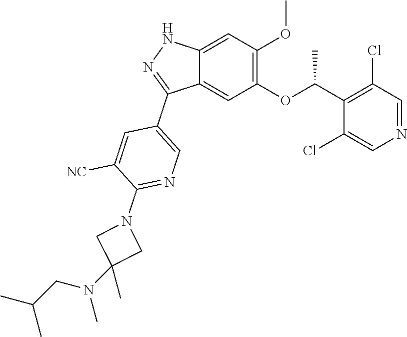 [SMILES: COc1cc2[nH]nc(-c3cnc(N4CC(C)(N(C)CC(C)C)C4)c(C#N)c3)c2cc1O[C@H](C)c1c(Cl)cncc1Cl] | (R)-5-(5-(1-(3,5- dichloropyridin-4- yl)ethoxy)-6- methoxy-1H- indazol-3-yl)-2-(3- (isobutyl(meth- yl)amino)-3- methylazetidin-1- yl)nicotinonitrile | LCMS m/z = 594.4 (M + H); 1HNMR (400 MHz, DMSO-d6) δ 12.98 (s, 1H), 8.72 (d, J = 2.4 Hz, 1H), 8.59 (s, 2H), 8.08 (d, J = 2.0 Hz, 1H), 7.04 (s, 1H), 7.01 (s, 1H), 6.00 (q, J = 6.8 Hz, 1H), 3.99 (s, 4H), 3.86 (s, 3H), 2.09 (s, 3H), 2.01 (d, J = 7.2 Hz, 2H), 1.76 (d, J = 6.8 Hz, 3H), 1.72-1.65 (m, 1H), 1.32 (s, 3H), 0.88 (d, J = 6.4 Hz, 6H) |
| Example 344. | 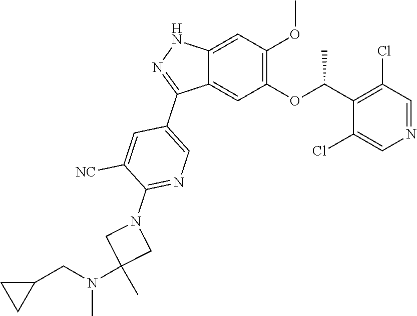 [SMILES: COc1cc2[nH]nc(-c3cnc(N4CC(C)(N(C)CC5CC5)C4)c(C#N)c3)c2cc1O[C@H](C)c1c(Cl)cncc1Cl] | (R)-2-(3- ((cyclopropyl- methyl) (methyl)amino)-3- methylazetidin-1- yl)-5-(5-(1-(3,5- dichloropyridin-4- yl)ethoxy)-6- methoxy-1H- indazol-3- yl)nicotinonitrile | LCMS m/z = 592.3 (M + H); 1HNMR (400 MHz, DMSO-d6) δ 12.99 (s, 1H), 8.72 (d, J = 1.6 Hz, 1H), 8.59(s, 2H), 8.08 (d, J = 2.4 Hz, 1H), 7.04 (s, 1H), 7.01 (s, 1H), 6.03 (q, J = 6.8 Hz, 1H), 4.05 (d, J = 8.0 Hz, 2H), 3.98 (d, J = 8.4 Hz, 2H), 3.86 (s, 3H), 2.20 (s, 3H), 2.15 (d, J = 6.4 Hz, 2H), 1.76 (d, J = 6.8 Hz, 3H), 1.30 (s, 3H), 0.87- 0.78 (m, 1H), 0.51-0.43 (m, 2H), 0.16-0.08 (m, 2H) |
| Example 345. | 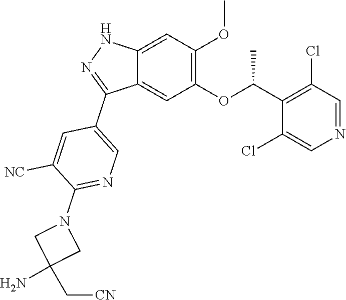 [SMILES: COc1cc2[nH]nc(-c3cnc(N4CC(N)(CC#N)C4)c(C#N)c3)c2cc1O[C@H](C)c1c(Cl)cncc1Cl] | 2-[3-amino-3- (cyanomethyl)azeti- din-1-yl]-5-[5-[(1R)- 1-(3,5-dichloro-4- pyridyl)ethoxy]-6- methoxy-1H- indazol-3- yl]pyridine-3- carbonitrile | LCMS m/z = 549.1 (M + H); 1HNMR (400 MHz, DMSO-d6) δ 13.05 (br s, 1H), 9.22 (br s, 3H), 8.79 (d, J = 2.3 Hz, 1H), 8.58 (s, 2H), 8.20 (d, J = 2.3 Hz, 1H), 7.07 (s, 1H), 7.02 (s, 1H), 6.00 (q, J = 6.7 Hz, 1H), 4.56 (d, J = 10.1 Hz, 2H), 4.44 (d, J = 10.1 Hz, 2H), 3.86 (s, 3H), 3.48 (s, 2H), 1.76 (d, J = 6.7 Hz, 3H) |
| Example 346. | 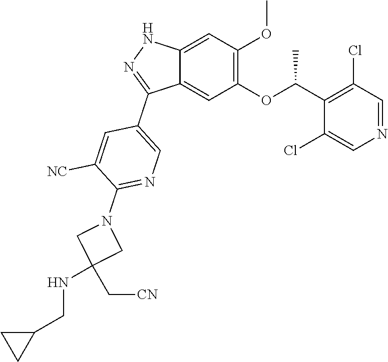 [SMILES: COc1cc2[nH]nc(-c3cnc(N4CC(CC#N)(NCC5CC5)C4)c(C#N)c3)c2cc1O[C@H](C)c1c(Cl)cncc1Cl] | (R)-2-(3- (cyanomethyl)-3- ((cyclo- propylmethyl)- amino)azetidin-1- yl)-5-(5-(1-(3,5- dichloropyridin-4- yl)ethoxy)-6- methoxy-1H- indazol-3- yl)nicotinonitrile | LCMS m/z = 603.4 (M + H); 1HNMR (400 MHz, DMSO-d6) δ 9.94 (brs, 1H), 8.76 (d, J = 2.2 Hz, 1H), 8.49 (s, 2H), 8.02 (d, J = 2.2 Hz, 1H), 7.02 (s, 1H), 6.90 (s, 1H), 5.99 (q, J = 6.8 Hz, 1H), 4.34-4.32 (m, 4H), 3.95 (s, 3H), 3.00 (s, 2H), 2.57 (d, J = 6.0 Hz, 2H), 1.86 (d, J = 7.2 Hz, 3H), 1.03-0.95 (m, 1H), 0.61-0.58 (m, 2H), 0.24-0.22 (m, 2H) |
| Example 347. | 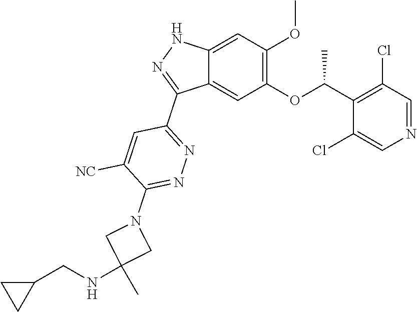 [SMILES: COc1cc2[nH]nc(-c3cc(C#N)c(N4CC(C)(NCC5CC5)C4)nn3)c2cc1O[C@H](C)c1c(Cl)cncc1Cl] | (R)-3-(3- ((cyclopropyl- methyl)amino)-3- methylazetidin-1- yl)-6-(5-(1-(3,5- dichloropyridin-4- yl)ethoxy)-6- methoxy-1H- indazol-3- yl)pyridazine-4- carbonitrile | LCMS m/z = 579.3 (M + H); 1HNMR (400 MHz, DMSO-d6) δ 13.18 (s, 1H), 8.59 (s, 2H), 8.26 (s, 1H), 7.70 (s, 1H), 7.03 (s, 1H), 5.91 (q, J = 6.8 Hz, 1H), 4.24 (d, J = 8.4 Hz, 2H), 4.12 (d, J = 8.4 Hz, 2H), 3.87 (s, 3H), 2.43 (d, J = 6.8 Hz, 2H), 1.75 (d, J = 6.4 Hz, 3H), 1.45 (s, 3H), 0.93-0.83 (m, 1H), 0.45-0.40 (m, 2H), 0.17-0.14 (m, 2H) |
| Example 348. | 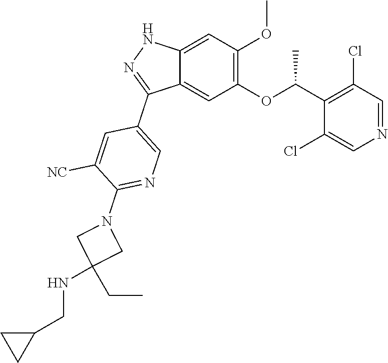 [SMILES: CCC1(NCC2CC2)CN(c2ncc(-c3n[nH]c4cc(OC)c(O[C@H](C)c5c(Cl)cncc5Cl)cc34)cc2C#N)C1] | 2-[3-(cyclo- propylmethyla- mino)-3-ethyl- azetidin-1-yl]-5-[5- [(1R)-1-(3,5- dichloro-4- pyridyl)ethoxy]-6- methoxy-1H- indazol-3- yl]pyridine-3- carbonitrile HCl | LCMS m/z = 592.2 (M + H); 1HNMR (400 MHz, DMSO-d6) δ 13.08 (br s, 1H), 9.84 (br s, 2H), 8.77 (d, J = 2.3 Hz, 1H), 8.59 (s, 2H), 8.17 (d, J = 2.3 Hz, 1H), 7.06 (s, 1H), 7.02 (s, 1H), 6.00 (q, J = 6.6 Hz, 1H), 4.54 (d, J = 10.0 Hz, 2H), 4.29 (d, J = 10.0 Hz, 2H), 3.86 (s, 3H), 2.95-2.81 (m, 2H), 2.00 (q, J = 7.3 Hz, 2H), 1.76 (d, J = 6.6 Hz, 3H), 1.18-1.10 (m, 1H), 1.06 (t, J = 7.4 Hz, 3H), 0.65-0.55 (m, 2H), 0.47-0.39 (m, 2H) |
| Example 349. | 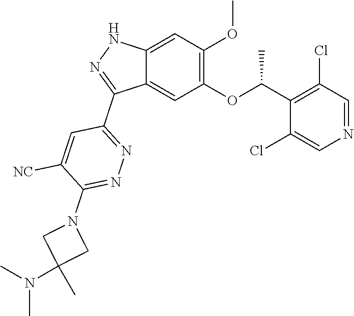 [SMILES: COc1cc2[nH]nc(-c3cc(C#N)c(N4CC(C)(N(C)C)C4)nn3)c2cc1O[C@H](C)c1c(Cl)cncc1Cl] | (R)-6-(5-(1-(3,5- dichloropyridin-4- yl)ethoxy)-6- methoxy-1H- indazol-3-yl)-3-(3- (dimethylamino)-3- methylazetidin-1- yl)pyridazine-4- carbonitrile | LCMS m/z = 553.3 (M + H); 1HNMR (400 MHz, DMSO-d6) δ 8.58 (s, 2H), 8.27 (s, 1H), 7.70 (s, 1H), 7.05 (s, 1H), 5.92 (q, J = 6.8 Hz, 1H), 4.20 (d, J = 8.8 Hz, 2H), 4.07 (d, J = 8.4 Hz, 2H), 3.87 (s, 3H), 2.16 (s, 6H), 1.75 (d, J = 6.8 Hz, 3H), 1.35 (s, 3H) |
| Example 350. | 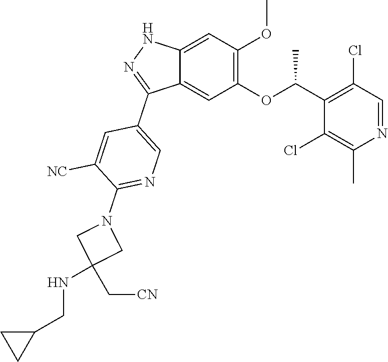 | (R)-2-(3- (Cyanomethyl)-3- ((cyclopropyl- methyl)ami- no)azetidin- 1-yl)-5-(5-(1-(3,5- dichloro-2- methylpyridin-4- yl)ethoxy)-6- methoxy-1H- indazol-3- yl)nicotinonitrile | LCMS m/z = 717.2 (M + H); 1HNMR (400 MHz, DMSO-d6) δ 12.96 (br s, 1H), 8.64 (s, 1H), 8.43 (s, 1H), 8.13 (s, 1H), 7.00 (s, 1H), 6.96 (s, 1H), 6.00 (q, J = 6.6 Hz, 1H), 4.18 (q, J = 9.0 Hz, 4H), 3.87 (s, 3H), 3.14 (s, 2H), 2.69 (br t, J = 7.0 Hz, 1H), 2.58 (s, 3H), 2.45-2.41 (m, 2H), 1.75 (d, J = 6.6 Hz, 3H), 0.93-0.83 (m, 1H), 0.46-0.40 (m, 2H), 0.20-0.14 (m, 2H) |
| Example 351. | 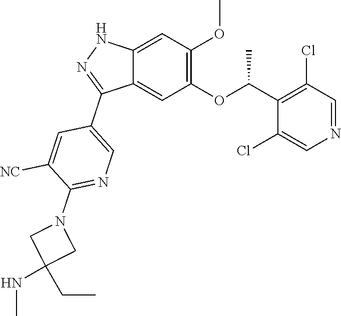 [SMILES: CCC1(NC)CN(c2ncc(-c3n[nH]c4cc(OC)c(O[C@H](C)c5c(Cl)cncc5Cl)cc34)cc2C#N)C1] | (R)-5-(5-(1-(3,5- Dichloropyridin-4- yl)ethoxy)-6- methoxy-1H- indazol-3-yl)-2-(3- ethyl-3-(methyl- amino)azeti- din-1- yl)nicotinonitrile diHCl | LCMS m/z = 552 (M + H); 1HNMR (400 MHz, DMSO-d6) δ 13.05 (br s, 1H), 9.72 (br d, J = 4.9 Hz, 2H), 8.77 (s, 1H), 8.59 (s, 2H), 8.18-8.16 (m, 1H), 7.06 (s, 1H), 7.02 (s, 1H), 6.00 (q, J = 6.6 Hz, 1H), 4.45 (d, J = 10.0 Hz, 2H), 4.31 (d, J = 10.0 Hz, 2H), 3.86 (s, 3H), 2.60 (t, J = 5.1 Hz, 3H), 2.00 (q, J = 7.3 Hz, 2H), 1.76 (d, J = 6.6 Hz, 3H), 1.04 (t, J = 7.3 Hz, 3H) |
| Example 352. | 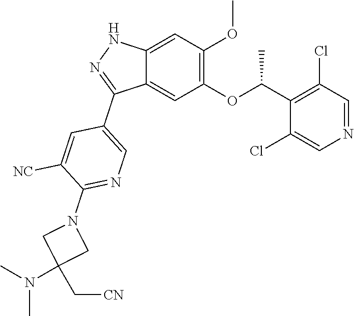 [SMILES: COc1cc2[nH]nc(-c3cnc(N4CC(CC#N)(N(C)C)C4)c(C#N)c3)c2cc1O[C@H](C)c1c(Cl)cncc1Cl] | (R)-2-(3- (cyanomethyl)-3- (dimethylamino)- azetidin-1-yl)-5-(5- (1-(3,5- dichloropyridin-4- yl)ethoxy)-6- methoxy-1H- indazol-3- yl)nicotinonitrile | LCMS m/z = 577.3 (M + H); 1HNMR (400 MHz, DMSO-d6) δ 13.00 (s, 1H), 8.75 (d, J = 2.4 Hz, 1H), 8.59 (s, 2H), 8.13 (d, J = 2.4 Hz, 1H), 7.06 (s, 1H), 7.01 (s, 1H), 6.01 (q, J = 6.4 Hz, 1H), 4.22 (s, 4H), 3.86 (s, 3H), 3.11 (s, 2H), 2.44 (s, 6H), 1.76 (d, J = 6.8 Hz, 3H) |
| Example 353. | 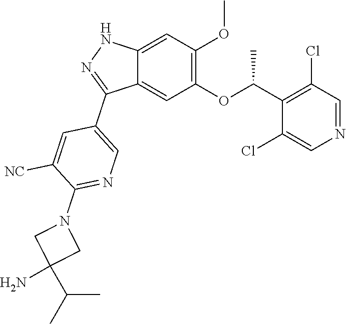 [SMILES: COc1cc2[nH]nc(-c3cnc(N4CC(N)(C(C)C)C4)c(C#N)c3)c2cc1O[C@H](C)c1c(Cl)cncc1Cl] | (R)-2-(3-amino-3- isopropylazetidin-1- yl)-5-(5-(1-(3,5- dichloropyridin-4- yl)ethoxy)-6- methoxy-1H- indazol-3- yl)nicotinonitrile | LCMS m/z = 552.3 (M + H); 1HNMR (400 MHz, DMSO-d6) δ 12.96 (s, 1H), 8.70 (d, J = 2.0 Hz, 1H), 8.59 (s, 2H), 8.05 (d, J = 2.4 Hz, 1H), 7.03 (s, 1H), 7.00 (s, 1H), 6.00 (q, J = 6.4 Hz, 1H), 4.16 (d, J = 8.8 Hz, 2H), 3.94 (d, J = 8.8 Hz, 2H), 3.86 (s, 3H), 1.97-1.90 (m, 1H), 1.76 (d, J = 6.4 Hz, 3H), 0.91 (d, J = 6.8 Hz, 6H) |
| Example 354. | 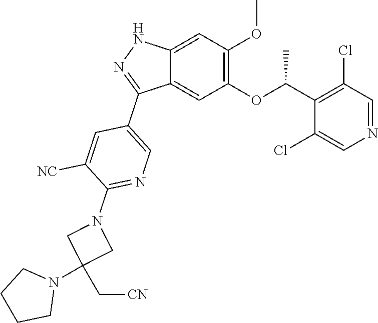 [SMILES: COc1cc2[nH]nc(-c3cnc(N4CC(CC#N)(N5CCCC5)C4)c(C#N)c3)c2cc1O[C@H](C)c1c(Cl)cncc1Cl] | (R)-2-(3- (cyanomethyl)-3- (pyrrolidin-1- yl)azetidin-1-yl)-5- (5-(1-(3,5- dichloropyridin-4- yl)ethoxy)-6- methoxy-1H- indazol-3- yl)nicotinonitrile | LCMS m/z = 603.4 (M + H); 1HNMR (400 MHz, DMSO-d6) δ 12.99 (s, 1H), 8.74 (d, J = 1.6 Hz, 1H), 8.59 (s, 2H), 8.12 (d, J = 1.6 Hz, 1H), 7.04 (s, 1H), 7.01 (s, 1H), 6.00 (q, J =6.4 Hz, 1H), 4.39 (d, J = 9.6 Hz, 2H), 4.17 (d, J = 9.2 Hz, 2H), 3.86 (s, 3H), 3.22 (s, 2H), 2.79-2.69 (m, 4H), 1.77-1.75 (m, 7H) |
| Example 355. | 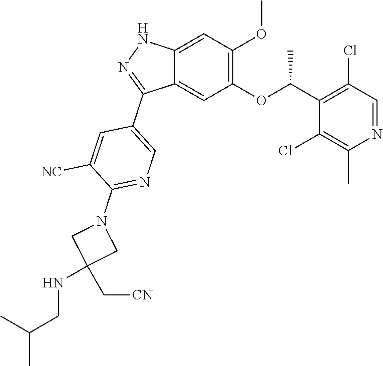 | 5-[5-[(1R)-1-(3,5- dichloro-2-methyl- 4- pyridyl)ethoxy]-6- methoxy-1H- indazol-3-yl]-2-[3- (isobutylamino)-3- methyl-azetidin-1- yl]pyridine-3- carbonitrile | LCMS m/z = 594.2 (M +H); 1HNMR (400 MHz, DMSO-d6) δ 12.87 (br s, 1H), 8.54 (s, 1H), 8.36 (s, 1H), 8.01 (s, 1H), 6.92 (s, 1H), 6.87 (s, 1H), 5.92 (q, J = 6.6 Hz, 1H), 4.03 (br d, J = 8.6 Hz, 2H), 3.94 (d, J = 8.4 Hz, 2H), 3.79 (s, 3H), 2.50 (s, 3H), 2.25 (br d, J = 6.2 Hz, 2H), 2.15 (br s, 1H), 1.67 (d, J = 6.6 Hz, 3H), 1.52 (quind, J = 6.5, 13.0 Hz, 1H), 1.34 (s, 3H), 0.83 (d, J = 6.5 Hz, 6H) |
| Example 356. | 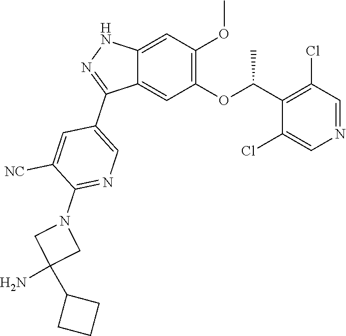 [SMILES: COc1cc2[nH]nc(-c3cnc(N4CC(N)(C5CCC5)C4)c(C#N)c3)c2cc1O[C@H](C)c1c(Cl)cncc1Cl] | (R)-2-(3-amino-3- cyclobutylazetidin- 1-yl)-5-(5-(1-(3,5- dichloropyridin-4- yl)ethoxy)-6- methoxy-1H- indazol-3- yl)nicotinonitrile | LCMS m/z = 564.3 (M + H); 1HNMR (400 MHz, DMSO-d6) δ 12.97 (s, 1H), 8.71 (d, J = 2.4 Hz, 1H), 8.59 (s, 2H), 8.06 (d, J = 2.4 Hz, 1H), 7.03 (s, 1H), 7.00 (s, 1H), 5.99 (q, J = 6.4 Hz, 1H), 4.20-4.15 (m, 2H), 3.99-3.94 (m, 2H), 3.86 (s, 3H), 2.67-2.63 (m, 1H), 1.98-1.86 (m, 6H), 1.76 (d, J = 6.4 Hz, 3H) |
| Example 357. | 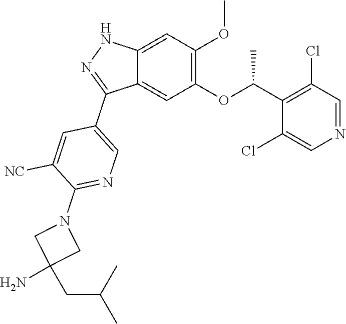 [SMILES: COc1cc2[nH]nc(-c3cnc(N4CC(N)(CC(C)C)C4)c(C#N)c3)c2cc1O[C@H](C)c1c(Cl)cncc1Cl] | (R)-2-(3-amino-3- isobutylazetidin-1- yl)-5-(5-(1-(3,5- dichloropyridin-4- yl)ethoxy)-6- methoxy-1H- indazol-3- yl)nicotinonitrile | LCMS m/z = 566.3 (M + H); 1HNMR (400 MHz, DMSO-d6) δ 12.96 (s, 1H), 8.70 (s, 1H), 8.59 (s, 2H), 8.05 (s, 1H), 7.03 (s, 1H), 7.00 (s, 1H), 6.00 (q, J = 6.4 Hz, 1H), 4.16 (d, J = 8.8 Hz, 2H), 4.03 (d, J = 8.4 Hz, 2H), 3.86 (s, 3H), 1.93-1.86 (m, 1H), 1.76 (d, J = 6.4 Hz, 3H), 1.64 (d, J = 6.4 Hz, 2H), 0.93 (d, J = 6.8 Hz, 6H) |
| Example 358. | 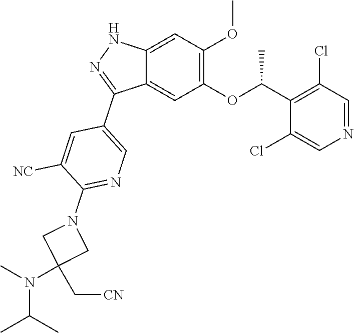 [SMILES: COc1cc2[nH]nc(-c3cnc(N4CC(CC#N)(N(C)C(C)C)C4)c(C#N)c3)c2cc1O[C@H](C)c1c(Cl)cncc1Cl] | (R)-2-(3- (cyanomethyl)-3- (isopropyl(methyl)- amino)azetidin-1- yl)-5-(5-(1-(3,5- dichloropyridin-4- yl)ethoxy)-6- methoxy-1H- indazol-3- yl)nicotinonitrile | LCMS m/z = 605.3 (M + H); 1HNMR (400 MHz, DMSO-d6) δ 13.00 (s, 1H), 8.74 (d, J = 2.4 Hz, 1H), 8.59 (s, 2H), 8.12 (d, J = 2.4 Hz, 1H), 7.05 (s, 1H), 7.01 (s, 1H), 6.03 (q, J = 6.4 Hz, 1H), 4.27-4.19 (m, 4H), 3.86 (s, 3H), 3.13 (s, 2H), 3.03- 2.96 (m, 1H), 2.18 (s, 3H), 1.77 (d, J = 6.4 Hz, 3H), 1.02 (d, J = 6.4 Hz, 6H) |
| Example 359. | 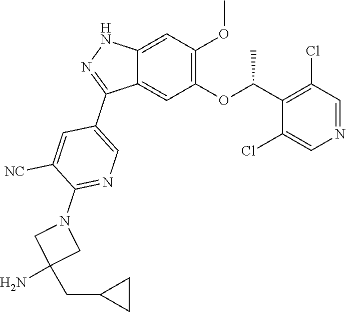 [SMILES: COc1cc2[nH]nc(-c3cnc(N4CC(N)(CC5CC5)C4)c(C#N)c3)c2cc1O[C@H](C)c1c(Cl)cncc1Cl] | R)-2-(3-amino-3- (cyclopropylmethyl) azetidin-1-yl)-5-(5- (1-(3,5- dichloropyridin-4- yl)ethoxy)-6- methoxy-1H- indazol-3- yl)nicotinonitrile | LCMS m/z = 564.3 (M + H); 1HNMR (400 MHz, DMSO-d6) δ 12.98 (brs, 1H), 8.72 (d, J = 2.4 Hz, 1H), 8.59 (s, 2H), 8.07 (d, J = 2.0 Hz, 1H), 7.03 (s, 1H), 7.00 (s, 1H), 5.99 (q, J = 6.8 Hz, 1H), 4.29 (d, J = 8.8 Hz, 2H), 4.08 (d, J = 9.2 Hz, 2H), 3.86 (s, 3H), 1.76 (d, J = 6.8 Hz, 3H), 1.66 (d, J = 5.1 Hz, 2H), 0.88-0.82 (m, 1H), 0.50- 0.45 (m, 2H), 0.18-0.14 (m, 2H) |
| Example 360. | 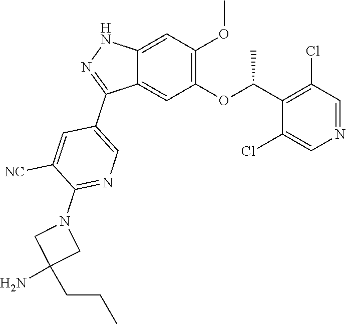 [SMILES: CCCC1(N)CN(c2ncc(-c3n[nH]c4cc(OC)c(O[C@H](C)c5c(Cl)cncc5Cl)cc34)cc2C#N)C1] | (R)-2-(3-amino-3- propylazetidin-1-yl)- 5-(5-(1-(3,5- dichloropyridin-4- yl)ethoxy)-6- methoxy-1H- indazol-3- yl)nicotinonitrile | LCMS m/z = 552.3 (M + H); 1HNMR (400 MHz, DMSO-d6) δ 12.96 (s, 1H), 8.71 (d, J = 2.4 Hz, 1H), 8.59 (s, 2H), 8.05 (d, J = 2.0 Hz, 1H), 7.03 (s, 1H), 7.00 (s, 1H), 5.99 (q, J = 6.8 Hz, 1H), 4.12 (d, J = 8.8 Hz, 2H), 3.97 (d, J = 8.8 Hz, 2H), 3.86 (s, 3H), 1.76 (d, J = 6.4 Hz, 3H), 1.68-1.64 (m, 2H), 1.44-1.38 (m, 2H), 0.94 (t, J = 7.2 Hz, 3H) |
| Example 361. | 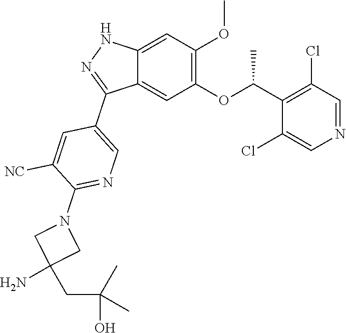 [SMILES: COc1cc2[nH]nc(-c3cnc(N4CC(N)(CC(C)(C)O)C4)c(C#N)c3)c2cc1O[C@H](C)c1c(Cl)cncc1Cl] | (R)-2-(3-amino-3- (2-hydroxy-2- methylpropyl) azetidin- 1-yl)-5-(5-(1-(3,5- dichloropyridin-4- yl)ethoxy)-6- methoxy-1H- indazol-3- yl)nicotinonitrile | LCMS m/z = 582.3 (M + H); 1HNMR (400 MHz, DMSO-d6) δ 12.99 (brs, 1H), 8.71 (d, J = 2.0 Hz, 1H), 8.58 (s, 2H), 8.05 (d, J = 2.4 Hz, 1H), 7.02-7.00 (m, 2H), 5.99 (q, J = 6.4 Hz, 1H), 4.29 (d, J = 9.2 Hz, 2H), 4.09 (d, J = 8.8 Hz, 2H), 3.86 (s, 3H), 1.90 (s, 2H), 1.76 (d, J = 6.4 Hz, 3H), 1.18 (s, 6H) |
| Example 362. | 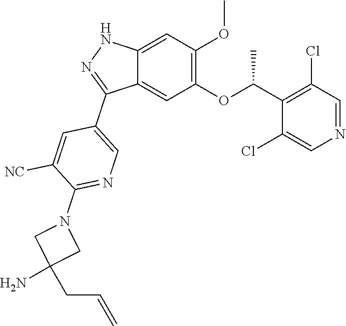 [SMILES: C=CCC1(N)CN(c2ncc(-c3n[nH]c4cc(OC)c(O[C@H](C)c5c(Cl)cncc5Cl)cc34)cc2C#N)C1] | (R)-2-(3-ally1-3- aminoazetidin-1-yl)- 5-(5-(1-(3,5- dichloropyridin-4- yl)ethoxy)-6- methoxy-1H- indazol-3- yl)nicotinonitrile | LCMS m/z = 550.3 (M + H); 1HNMR (400 MHz, DMSO-d6) δ 12.96 (s, 1H), 8.71 (d, J = 2.4 Hz, 1H), 8.60 (s, 2H), 8.05 (d, J = 2.0 Hz, 1H), 7.03 (s, 1H), 7.00 (s, 1H), 6.02-5.92 (m, 2H), 5.22-5.14 (m, 2H), 4.17 (d, J = 8.8 Hz, 2H), 3.99 (d, J = 8.8 Hz, 2H), 3.86 (s, 3H), 2.45 (d, J = 7.2 Hz, 2H), 1.76 (d, J = 6.4 Hz, 3H) |
| Example 363. | 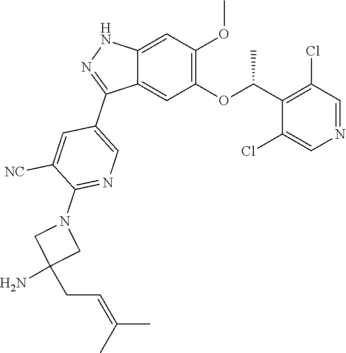 [SMILES: COc1cc2[nH]nc(-c3cnc(N4CC(N)(CC=C(C)C)C4)c(C#N)c3)c2cc1O[C@H](C)c1c(Cl)cncc1Cl] | (R)-2-(3-amino-3- (3-methylbut-2-en- 1-yl)azetidin-1-yl)- 5-(5-(1-(3,5- dichloropyridin-4- yl)ethoxy)-6- methoxy-1H- indazol-3- yl)nicotinonitrile | LCMS m/z = 578.4 (M + H); 1HNMR (400 MHz, DMSO-d6) δ 12.96 (brs, 1H), 8.71 (d, J = 2.4 Hz, 1H), 8.58 (s, 2H), 8.06 (d, J = 2.8 Hz, 1H), 7.03 (s, 1H), 7.00 (s, 1H), 6.00 (q, J = 6.4 Hz, 1H), 5.29 (t, J = 7.6 Hz, 1H), 4.14 (d, J = 8.8 Hz, 2H), 4.01 (d, J = 8.4 Hz, 2H), 3.86 (s, 3H), 2.41 (d, J = 7.2 Hz, 2H), 1.76 (d, J = 6.4 Hz, 3H), 1.71 (s, 3H), 1.66 (s, 3H) |
| Example 364. | 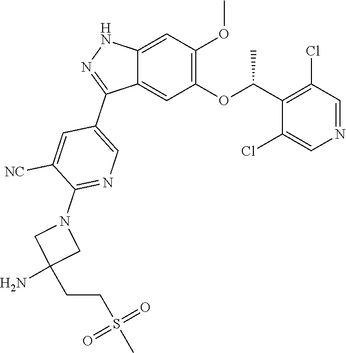 [SMILES: COc1cc2[nH]nc(-c3cnc(N4CC(N)(CCS(C)(=O)=O)C4)c(C#N)c3)c2cc1O[C@H](C)c1c(Cl)cncc1Cl] | (R)-2-(3-amino-3- (2-(methyl- sulfonyl)ethyl) azetidin-1-yl)-5-(5- (1-(3,5- dichloropyridin-4- yl)ethoxy)-6- methoxy-1H- indazol-3- yl)nicotinonitrile | LCMS m/z = 616.3 (M + H); 1HNMR (400 MHz, DMSO-d6) δ 12.98 (brs, 1H), 8.72 (d, J = 2.4 Hz, 1H), 8.59 (s, 2H), 8.06 (d, J = 2.0 Hz, 1H), 7.03 (s, 1H), 7.01 (s, 1H), 5.99 (q, J = 6.4 Hz, 1H), 4.20 (d, J = 8.8 Hz, 2H), 4.00 (d, J = 8.8 Hz, 2H), 3.86 (s, 3H), 3.31-3.23 (m, 2H), 3.02 (s, 3H), 2.42 (brs, 2H), 2.18-2.10 (m, 2H), 1.76 (d, J = 6.8 Hz, 3H) |
| Example 365. | 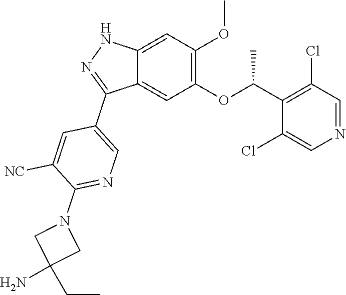 [SMILES: CCC1(N)CN(c2ncc(-c3n[nH]c4cc(OC)c(O[C@H](C)c5c(Cl)cncc5Cl)cc34)cc2C#N)C1] | 2-(3-amino-3-ethyl- azetidin-1-yl)-5-[5- [(1R)-1-(3,5- dichloro-4- pyridyl)ethoxy]-6- methoxy-1H- indazol-3- yl]pyridine-3- carbonitrile | LCMS m/z = 538 (M + H); 1HNMR (400 MHz, DMSO-d6) δ 12.96 (br s, 1H), 8.71 (d, J = 2.0 Hz, 1H), 8.59 (s, 2H), 8.05 (d, J = 2.0 Hz, 1H), 7.03 (s, 1H), 7.00 (s, 1H), 5.99 (q, J = 6.4 Hz, 1H), 4.11 (br d, J = 8.6 Hz, 2H), 3.97 (br d, J = 8.6 Hz, 2H), 3.86 (s, 3H), 2.21 (br s, 2H), 1.76 (br d, J = 6.6 Hz, 3H), 1.70 (q, J = 7.1 Hz, 2H), 0.94 (br t, J = 7.3 Hz, 3H) |
| Example 366. | 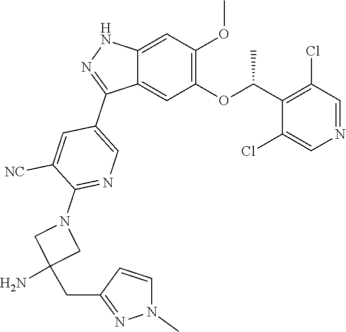 [SMILES: COc1cc2[nH]nc(-c3cnc(N4CC(N)(Cc5ccn(C)n5)C4)c(C#N)c3)c2cc1O[C@H](C)c1c(Cl)cncc1Cl] | (R)-2-(3-amino-3- ((1-methyl-1H- pyrazol-3- yl)methyl)azetidin- 1-yl)-5-(5-(1-(3,5- dichloropyridin-4- yl)ethoxy)-6- methoxy-1H- indazol-3- yl)nicotinonitrile | LCMS m/z = 604.3 (M + H); 1HNMR (400 MHz, DMSO-d6) δ (s, 1H), 8.69 (s, 1H), 8.59 (s, 2H)), 8.04 (s, 1H), 7.57 (s, 1H), 7.03-7.00 (m, 2H), 6.17 (s, 1H), 5.99 (q, J = 6.0 Hz, 1H), 4.31 (d, J = 8.8 Hz, 2H), 4.01 (d, J = 8.8 Hz, 2H), 3.86 (s, 3H), 3.78 (s, 3H), 2.95 (s, 2H) , 1.76 (d, J = 6.8 Hz, 3H) |
| Example 367. | 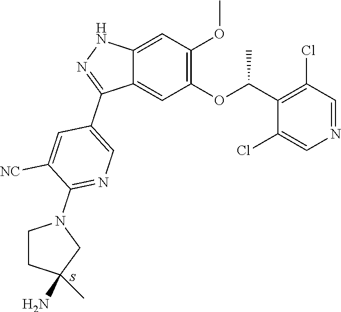 | 2-((S)-3-amino-3- methylpyrrolidin-1- yl)-5-(5-((R)-1-(3,5- dichloropyridin-4- yl)ethoxy)-6- methoxy-1H- indazol-3- yl)nicotinonitrile | LCMS m/z = 538.2 (M + H); 1HNMR (400 MHz, DMSO-d6) δ 13.01 (brs, 1H), 8.74 (d, J = 2.0 Hz, 1H), 8.59 (s, 2H), 8.08 (d, J = 2.0 Hz, 1H), 7.03 (s, 1H), 7.01 (s, 1H), 6.03 (q, J = 6.4 Hz, 1H), 4.05-3.99 (m, 1H), 3.94-3.89 (m, 2H), 3.86 (s, 3H), 3.68 (d, J = 11.6 Hz, 1H), 2.18- 2.03 (m, 2H), 1.77 (d, J = 6.8 Hz, 3H), 1.43 (s, 3H) |
| Example 368. | 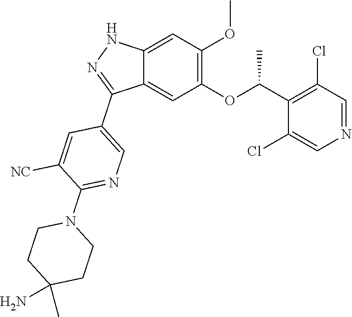 [SMILES: COc1cc2[nH]nc(-c3cnc(N4CCC(C)(N)CC4)c(C#N)c3)c2cc1O[C@H](C)c1c(Cl)cncc1Cl] | (R)-2-(4-amino-4- methylpiperidin-1- yl)-5-(5-(1-(3,5- dichloropyridin-4- yl)ethoxy)-6- methoxy-1H- indazol-3- yl)nicotinonitrile | LCMS m/z = 552.3 (M + H); 1HNMR (400 MHz, DMSO-d6) δ 13.02 (s, 1H), 8.76 (d, J = 2.4 Hz, 1H), 8.59 (s, 2H), 8.14 (d, J = 2.4 Hz, 1H), 7.06 (s, 1H), 7.01 (s, 1H), 6.00 (q, J = 6.8 Hz, 1H), 3.86 (s, 3H), 3.84-3.80 (m, 2H), 3.76-3.69 (m, 2H), 1.76 (d, J = 6.4 Hz, 3H), 1.64- 1.52 (m, 4H), 1.16 (s, 3H) |
| Example 369. | 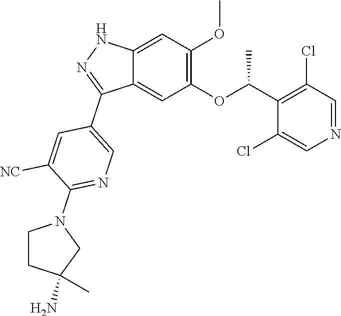 [SMILES: COc1cc2[nH]nc(-c3cnc(N4CC[C@@](C)(N)C4)c(C#N)c3)c2cc1O[C@H](C)c1c(Cl)cncc1Cl] | 2-((R)-3-amino-3- methylpyrrolidin-1- yl)-5-(5-((R)-1-(3,5- dichloropyridin-4- yl)ethoxy)-6- methoxy-1H- indazol-3- yl)nicotinonitrile | LCMS m/z = 538.2 (M + H); 1HNMR (400 MHz, DMSO-d6) δ 12.95 (brs, 1H), 8.71 (d, J = 2.4 Hz, 1H), 8.59 (s, 2H), 8.03 (d, J = 2.4 Hz, 1H), 7.02 (s, 1H), 7.00 (s, 1H), 6.00 (q, J = 6.4 Hz, 1H), 3.92-3.89 (m, 1H), 3.86 (s, 3H), 3.84-3.79 (m, 1H), 3.64-3.54 (m, 2H), 1.88-1.85 (m, 2H), 1.76 (d, J = 6.4 Hz, 3H), 1.29 (s, 3H) |
| Example 370. | 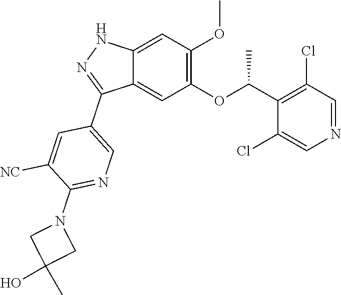 | 5-[5-[(1R)-1-(3,5- dichloro-4- pyridyl)ethoxy]-6- methoxy-1H- indazol-3-yl]-2-(3- hydroxy-3-methyl- azetidin-1- yl)pyridine-3- carbonitrile | LCMS m/z = 538.2 (M + H); 1HNMR (400 MHz, DMSO-d6) δ |
| Example 371. | 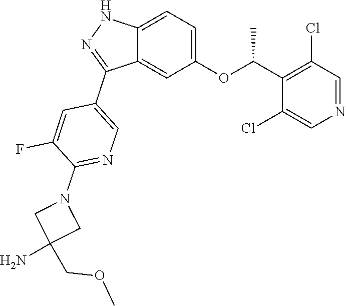 [SMILES: COCC1(N)CN(c2ncc(-c3n[nH]c4ccc(O[C@H](C)c5c(Cl)cncc5Cl)cc34)cc2F)C1] | 1-[5-[5-[(1R)-1-(3,5- dichloro-4- pyridyl)ethoxy]-1H- indazol-3-yl]-3- fluoro-2-pyridyl]-3- (methoxy- methyl)aze- tidin-3-amine | LCMS m/z = 517 (M + H); 1HNMR (400 MHz, DMSO-d6) δ 8.89 (br s, 3H), 8.57 (s, 2H), 8.41 (s, 1H), 7.80 (dd, J = 1.5, 13.1 Hz, 1H), 7.49 (d, J = 9.0 Hz, 1H), 7.20 (d, J = 2.1 Hz, 1H), 7.11 (dd, J = 2.1, 9.0 Hz, 1H), 6.14 (q, J = 6.6 Hz, 1H), 4.29-4.27 (m, 2H), 4.22-4.20 (m, 2H), 3.82 (s, 2H), 3.42 (s, 3H), 1.76 (d, J = 6.6 Hz, 3H) |
| Example 372. | 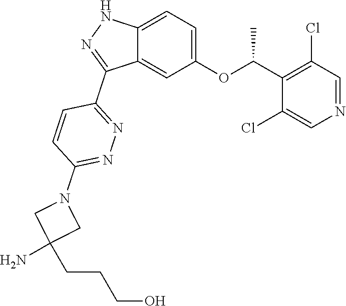 [SMILES: C[C@@H](Oc1ccc2[nH]nc(-c3ccc(N4CC(N)(CCCO)C4)nn3)c2c1)c1c(Cl)cncc1Cl] | (R)-3-(3-amino-1- (6-(5-(1-(3,5- dichloropyridin-4- yl)ethoxy)-1H- indazol-3- yl)pyridazin-3- yl)azetidin-3- yl)propan-1-ol | LCMS m/z = 514.2 (M + H); 1HNMR (400 MHz, DMSO-d6) δ 13.18 (s, 1H), 8.56 (s, 2H), 7.97 (s, 1H), 7.95 (d, J = 4.0 Hz, 1H), 7.46 (d, J = 9.2 Hz, 1H), 7.09 (dd, J = 8.8 Hz, 2.4 Hz, 1H), 6.88 (d, J = 9.2 Hz, 1H), 6.08 (q, J = 7.2 Hz, 1H), 3.98 (d, J = 8.4 Hz, 2H), 3.82 (d, J = 7.6 Hz, 2H), 3.46 (t, J = 6.4 Hz, 2H), 1.78-1.74 (m, 5H), 1.62-1.55 (m, 2H) |
| Example 373. | 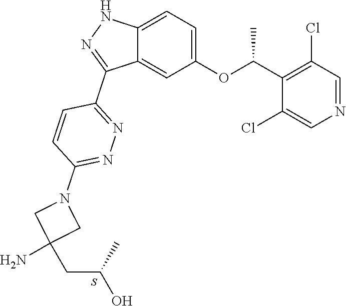 | (S)-1-(3-amino-1-(6- (5-((R)-1-(3,5- dichloropyridin-4- yl)ethoxy)-1H- indazol-3- yl)pyridazin-3- yl)azetidin-3- yl)propan-2-ol | LCMS m/z = 514.2 (M + H); 1HNMR (400 MHz, DMSO-d6) δ 13.17 (brs, 1H), 8.56 (s, 2H), 7.97-7.94 (m, 2H), 7.46 (d, J = 8.8 Hz, 1H), 7.09 (dd, J = 8.8 Hz, 2.4 Hz, 1H), 6.86 (d, J = 9.2 Hz, 1H), 6.08 (q, J = 6.8 Hz, 1H), 4.07 (d, J = 8.4 Hz, 2H), 4.01-3.96 (m, 1H), 3.87 (d, J = 8.0 Hz, 1H), 3.80 (d, J = 8.0 Hz, 1H), 1.79-1.76 (m, 5H), 1.12 (d, J = 6.4 Hz, 3H) |
| Example 374. | 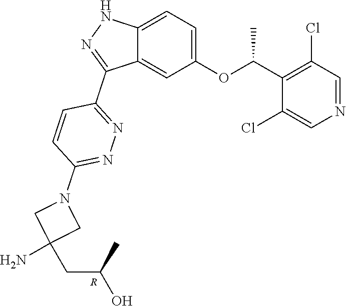 | (R)-1-(3-amino-1- (6-(5-((R)-1-(3,5- dichloropyridin-4- yl)ethoxy)-1H- indazol-3- yl)pyridazin-3- yl)azetidin-3- yl)propan-2-ol | LCMS m/z = 514.3 (M + H); 1HNMR (400 MHz, DMSO-d6) δ 13.19 (brs, 1H), 8.56 (s, 2H), 8.01-7.93 (m, 2H), 7.47 (d, J = 8.8 Hz, 1H), 7.10 (dd, J = 8.8 Hz, 2.0 Hz, 1H), 6.91 (d, J = 9.6 Hz, 1H), 6.08 (q, J = 6.8 Hz, 1H), 4.87 (brs, 2H), 4.18-4.10 (m, 2H), 4.02-3.88 (m, 3H), 1.86-1.76 (m, 5H), 1.14 (d, J = 7.6 Hz, 3H) |
| Example 375. | 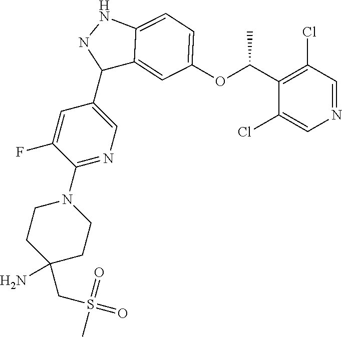 [SMILES: C[C@@H](Oc1ccc2c(c1)C(c1cnc(N3CCC(N)(CS(C)(=O)=O)CC3)c(F)c1)NN2)c1c(Cl)cncc1Cl] | (R)-1-(5-(5-(1-(3,5- dichloropyridin-4- yl)ethoxy)-1H- indazol-3-yl)-3- fluoropyridin-2-yl)- 4- ((methylsulfonyl) methyl)piperidin-4- amine | LCMS m/z = 593.3 (M + H); 1HNMR (400 MHz, DMSO-d6) δ 13.17 (s, 1H), 8.58 (s, 2H), 8.44 (s, 1H), 7.75 (dd, J = 14.4 Hz, 1.6 Hz, 1H), 7.49 (d, J = 9.2 Hz, 1H), 7.21 (d, J = 2.0 Hz, 1H), 7.11 (dd, J = 8.8 Hz, 16 Hz, 1H), 6.14 (q, J = 6.8 Hz, 1H), 3.75-3.69 (m, 2H), 3.53- 3.46 (m, 2H), 3.36 (s, 2H), 3.08 (s, 3H), 1.91-1.85 (m, 2H), 1.76 (d, J = 6.4 Hz, 3H), 1.73-1.70 (m, 2H) |
| Example 376. | 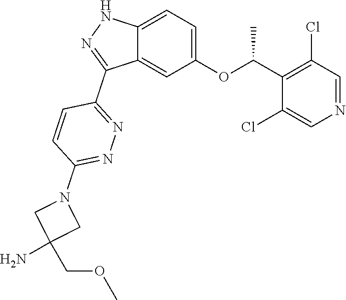 [SMILES: COCC1(N)CN(c2ccc(-c3n[nH]c4ccc(O[C@H](C)c5c(Cl)cncc5Cl)cc34)nn2)C1] | (R)-1-(6-(5-(1-(3,5- dichloropyridin-4- yl)ethoxy)-1H- indazol-3- yl)pyridazin-3-yl)-3- (methoxy- methyl)aze- tidin-3-amine | LCMS m/z = 500.2 (M + H); 1HNMR (400 MHz, DMSO-d6) δ 13.18 (s, 1H), 8.56 (s, 2H), 7.97- 7.94 (m, 2H), 7.46 (d, J = 9.2 Hz, 1H), 7.10 (dd, J = 8.8 Hz, 2.4 Hz, 1H), 6.89 (d, J = 9.2 Hz, 1H), 6.08 (q, J = 6.8 Hz, 1H), 4.05 (d, J = 8.0 Hz, 2H), 3.82 (d, J = 8.0 Hz, 2H), 3.49 (s, 2H), 3.37 (s, 3H), 3.01 (brs, 2H), 1.77 (d, J = 6.4 Hz, 3H) |
| Example 377. | 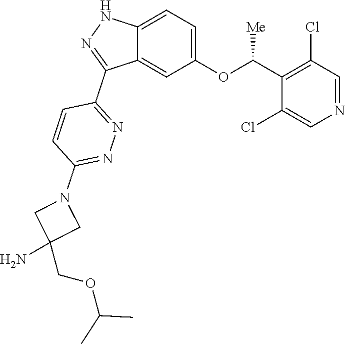 [SMILES: CC(C)OCC1(N)CN(c2ccc(-c3n[nH]c4ccc(O[C@H](C)c5c(Cl)cncc5Cl)cc34)nn2)C1] | (R)-1-(6-(5-(1-(3,5- dichloropyridin-4- yl)ethoxy)-1H- indazol-3- yl)pyridazin-3-yl)-3- (isopropoxy- methyl)a- zetidin-3-amine | LCMS m/z = 528.5 (M + H); 1HNMR (400 MHz, DMSO-d6) δ 13.18 (s, 1H), 8.55 (s, 2H), 7.98 (d, J = 9.2 Hz, 1H), 7.94 (d, J = 2.0 Hz, 1H), 7.47 (d, J = 8.8 Hz, 1H), 7.09 (dd, J = 9.2 Hz, 2.0 Hz, 1H), 6.92 (dd, J = 9.2 Hz, 3.6 Hz, 1H), 6.08 (q, J = 6.4 Hz, 1H), 4.15-4.01 (m, 2H), 3.94-3.82 (m, 2H), 3.69-3.62 (m, 1H), 3.58 (d, J = 5.2 Hz, 2H), 1.77 (d, J = 6.4 Hz, 3H), 1.14 (d, J = 6.4 Hz, 6H) |
| Example 378. | 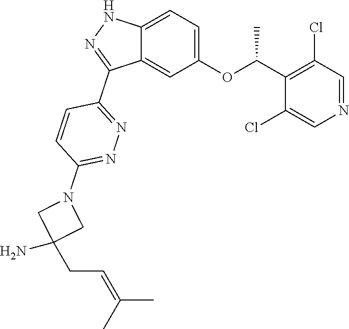 [SMILES: CC(C)=CCC1(N)CN(c2ccc(-c3n[nH]c4ccc(O[C@H](C)c5c(Cl)cncc5Cl)cc34)nn2)C1] | (R)-1-(6-(5-(1-(3,5- dichloropyridin-4- yl)ethoxy)-1H- indazol-3- yl)pyridazin-3-yl)-3- (3-methylbut-2-en- 1-yl)azetidin-3- amine | LCMS m/z = 524.3 (M + H); 1HNMR (400 MHz, DMSO-d6) δ 13.17 (s, 1H), 8.56 (s, 2H), 7.96-7.94 (m, 2H), 7.47 (d, J = 8.8 Hz, 1H), 7.10 (dd, J = 9.2 Hz, 2.4 Hz, 1H), 6.88 (d, J = 8.8 Hz, 1H), 6.09 (q, J = 6.8 Hz, 1H), 5.32 (t, J = 7.0 Hz, 1H), 3.97 (d, J = 8.0 Hz, 2H), 3.80 (d, J = 8.0 Hz, 2H), 2.42 (d, J = 7.2 Hz, 2H), 1.78 (d, J = 14.8 Hz, 3H), 1.71 (s, 3H), 1.66 (s, 3H) |
| Example 379. | 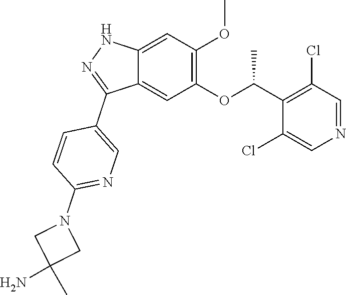 [SMILES: COc1cc2[nH]nc(-c3ccc(N4CC(C)(N)C4)nc3)c2cc1O[C@H](C)c1c(Cl)cncc1Cl] | (R)-1-(5-(5-(1-(3,5- dichloropyridin-4- yl)ethoxy)-6- methoxy-1H- indazol-3-yl)pyridin- 2-yl)-3- methylazetidin-3- amine | LCMS m/z = 499.3 (M + H); 1HNMR (400 MHz, DMSO-d6) δ 12.79 (brs, 1H), 8.61 (s, 2H), 8.44 (d, J = 2.0 Hz, 1H), 7.77 (dd, J = 8.8 Hz, 2.4 Hz, 1H), 7.06 (s, 1H), 6.99 (s, 1H), 6.49 (d, J = 8.4 Hz, 1H), 5.96 (q, J = 6.8 Hz, 1H), 3.86 (s, 3H), 3.82 (d, J = 8.0 Hz, 2H), 3.72 (d, J = 7.6 Hz, 2H), 1.76 (d, J = 6.8 Hz, 3H), 1.42 (s, 3H) |
| Example 380. | 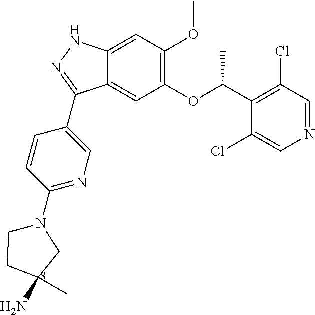 | (R)-1-(5-(5-((R)-1- (3,5-dichloropyridin- 4-yl)ethoxy)-6- methoxy-1H- indazol-3-yl)pyridin- 2-yl)-3- methylpyrrolidin-3- amine | LCMS m/z = 513.4 (M + H); 1HNMR (400 MHz, DMSO-d6) δ 12.75 (brs, 1H), 8.61 (s, 2H), 8.43 (d, J = 2.4 Hz, 1H), 7.75 (dd, J = 8.8 Hz, 2.4 Hz, 1H), 7.06 (s, 1H), 6.98 (s, 1H), 6.51 (d, J = 8.8 Hz, 1H), 5.96 (q, J = 6.8 Hz, 1H), 3.86 (s, 3H), 3.60- 3.47 (m, 4H), 1.88-1.85 (m, 2H), 1.76 (d, J = 6.8 Hz, 3H), 1.28 (s, 3H) |
| Example 381. | 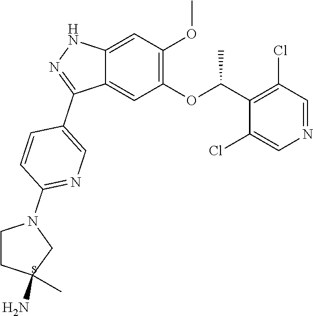 | (S)-1-(5-(5-((R)-1- (3,5-dichloropyridin- 4-yl)ethoxy)-6- methoxy-1H- indazol-3-yl)pyridin- 2-yl)-3-methyl- pyrrolidin-3-amine | LCMS m/z = 513.4 (M + H); 1HNMR (400 MHz, DMSO-d6) δ 12.74 (brs, 1H), 8.62 (s, 2H), 8.43 (d, J = 2.4 Hz, 1H), 7.76 (dd, J = 8.8 Hz, 2.4 Hz, 1H), 7.05 (s, 1H), 6.98 (s, 1H), 6.52 (d, J = 8.8 Hz, 1H), 5.98 (q, J = 6.4 Hz, 1H), 3.86 (s, 3H), 3.60- 3.55 (m, 1H), 3.53-3.50 (m, 1H), 3.29-3.27 (m, 2H), 1.88 (t, J = 6.8 Hz, 2H), 1.76 (d, J = 6.8 Hz, 3H), 1.28 (s, 3H) |
| Example 382. | 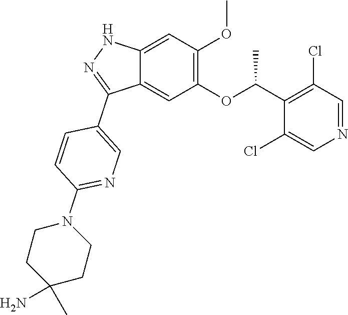 [SMILES: COc1cc2[nH]nc(-c3ccc(N4CCC(C)(N)CC4)nc3)c2cc1O[C@H](C)c1c(Cl)cncc1Cl] | (R)-1-(5-(5-(1-(3,5- dichloropyridin-4- yl)ethoxy)-6- methoxy-1H- indazol-3-yl)pyridin- 2-yl)-4- methylpiperidin-4- amine | LCMS m/z = 527.3 (M + H); 1HNMR (400 MHz, DMSO-d6) δ 12.79 (s, 1H), 8.62 (s, 2H), 8.45 (d, J = 2.4 Hz, 1H), 7.75 (dd, J = 8.8 Hz, 2.4 Hz, 1H), 7.04 (s, 1H), 6.98 (s, 1H), 6.93 (d, J = 9.2 Hz, 1H), 5.96 (q, J = 6.8 Hz, 1H), 3.86 (s, 3H), 3.77- 3.68 (m, 2H), 3.58-3.51 (m, 2H), 1.76 (d, J = 6.4 Hz, 3H), 1.58-1.41 (m, 4H), 1.09 (s, 3H) |
| Example 383. | 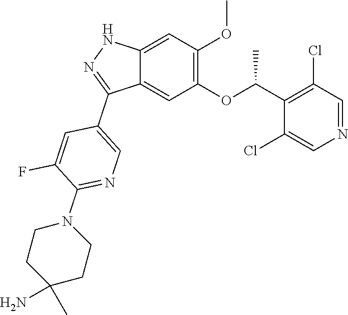 [SMILES: COc1cc2[nH]nc(-c3cnc(N4CCC(C)(N)CC4)c(F)c3)c2cc1O[C@H](C)c1c(Cl)cncc1Cl] | (R)-1-(5-(5-(1-(3,5- dichloropyridin-4- yl)ethoxy)-6- methoxy-1H- indazol-3-yl)-3- fluoropyridin-2-yl)- 4-methylpiperidin-4- amine | LCMS m/z = 545.3 (M + H); 1HNMR (400 MHz, DMSO-d6) δ 12.97 (brs, 1H), 8.59 (s, 2H), 8.36 (s, 1H), 7.66 (dd, J = 14.8 Hz, 1.6 Hz, 1H), 7.08 (s, 1H), 7.00 (s, 1H), 5.98 (q, J = 6.8 Hz, 1H), 3.86 (s, 3H), 3.61-3.46 (m, 4H), 1.76 (d, J = 6.8 Hz, 3H), 1.61-1.44 (m, 4H), 1.10 (s, 3H) |
| Example 384. | 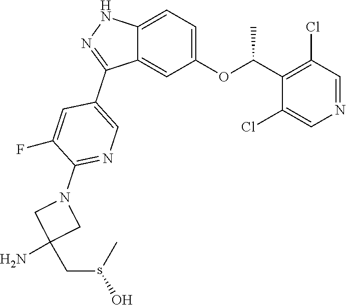 | (S)-1-(3-amino-1-(5- (5-((R)-1-(3,5- dichloropyridin-4- yl)ethoxy)-1H- indazol-3-yl)-3- fluoropyridin-2- yl)azetidin-3- yl)propan-2-ol | LCMS m/z = 531.4 (M + H); 1HNMR (400 MHz, DMSO-d6) δ 13.11 (brs, 1H), 8.57 (s, 2H), 8.36 (t, J = 1.6 Hz, 1H), 7.70 (dd, J = 13.2 Hz, 1.6 Hz, 1H), 7.47 (d, J = 8.8 Hz, 1H), 7.19 (d, J = 2.4 Hz, 1H), 7.09 (dd, J = 8.8 Hz, 2.0 Hz, 1H), 6.14 (q, J = 6.8 Hz, 1H), 4.11-4.07 (m, 2H), 3.98-3.92 (m, 1H), 3.92 (d, J = 8.4 Hz, 1H), 3.85 (d, J = 7.2 Hz, 1H), 1.79-1.75 (m, 5H), 1.12 (d, J = 6.4 Hz, 3H) |
| Example 385. | 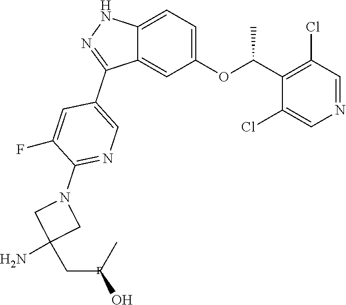 | (R)-1-(3-amino-1- (5-(5-((R)-1-(3,5- dichloropyridin-4- yl)ethoxy)-1H- indazol-3-yl)-3- fluoropyridin-2- yl)azetidin-3- yl)propan-2-ol | LCMS m/z = 531.3 (M + H); 1HNMR (400 MHz, DMSO-d6) δ 13.11 (brs, 1H), 8.57 (s, 2H), 8.36 (s, 1H), 7.70 (d, J = 13.2 Hz, 1H), 7.47 (d, J = 8.8 Hz, 1H), 7.19 (s, 1H), 7.09 (dd, J = 9.2 Hz, 1.6 Hz, 1H), 6.14 (q, J = 6.4 Hz, 1H), 4.10-4.04 (m, 2H), 4.01-3.95 (m, 1H), 3.92 (d, J = 8.4 Hz, 1H), 3.85 (d, J = 8.4 Hz, 1H), 1.79-1.75 (m, 5H), 1.12 (d, J = 6.0 Hz, 3H) |
| Example 386. | 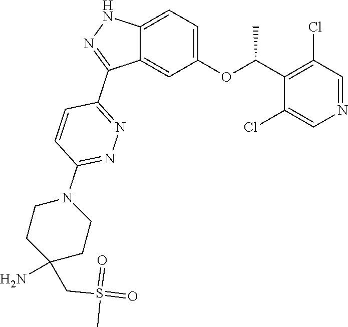 [SMILES: C[C@@H](Oc1ccc2[nH]nc(-c3ccc(N4CCC(N)(CS(C)(=O)=O)CC4)nn3)c2c1)c1c(Cl)cncc1Cl] | (R)-1-(6-(5-(1-(3,5- dichloropyridin-4- yl)ethoxy)-1H- indazol-3- yl)pyridazin-3-yl)-4- ((methylsulfonyl) methyl)piperidin-4- amine | LCMS m/z = 576.3 (M + H); 1HNMR (400 MHz, DMSO-d6) δ 13.16 (s, 1H), 8.56 (s, 2H), 7.95-7.92 (m, 2H), 7.48 (d, J = 8.8 Hz, 1H), 7.37 (d, J = 9.6 Hz, 1H), 7.10 (dd, J = 8.8 Hz, 2.8 Hz, 1H), 6.08 (q, J = 6.4 Hz, 1H), 4.03-3.98 (m, 2H), 3.59 (t, J = 10.4 Hz, 2H), 3.35 (s, 2H), 3.07 (s, 3H), 1.98 (brs, 2H), 1.86-1.78 (m, 2H), 1.76 (d, J = 6.8 Hz, 3H), 1.72-1.68 (m, 2H). |
| Example 387. | 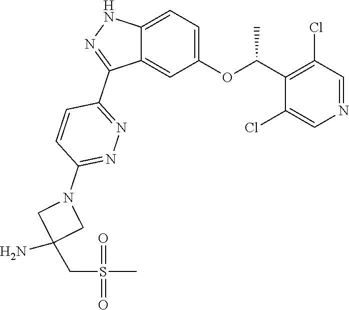 [SMILES: C[C@@H](Oc1ccc2[nH]nc(-c3ccc(N4CC(N)(CS(C)(=O)=O)C4)nn3)c2c1)c1c(Cl)cncc1Cl] | (R)-1-(6-(5-(1-(3,5- dichloropyridin-4- yl)ethoxy)-1H- indazol-3- yl)pyridazin-3-yl)-3- ((methylsulfonyl)meth- yl)azetidin-3- amine | LCMS m/z = 548.2 (M + H); 1HNMR (400 MHz, DMSO-d6) δ 13.19 (s, 1H), 8.56 (s, 2H), 7.97 (d, J = 9.2 Hz, 1H), 7.94 (d, J = 2.4 Hz, 1H), 7.47 (d, J = 9.2 Hz, 1H), 7.09 (dd, J = 8.8 Hz, 2.0 Hz, 1H), 6.92 (d, J = 8.8 Hz, 1H), 6.08 (q, J = 6.4 Hz, 1H), 4.31 (d, J = 8.4 Hz, 2H), 3.91 (d, J = 7.6 Hz, 2H), 3.70 (s, 2H), 3.13 (s, 3 H), 2.82 (brs, 2H), 1.76 (d, J = 6.4 Hz, 3H) |
| Example 388. | 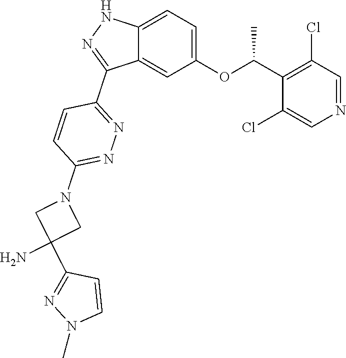 [SMILES: C[C@@H](Oc1ccc2[nH]nc(-c3ccc(N4CC(N)(c5ccn(C)n5)C4)nn3)c2c1)c1c(Cl)cncc1Cl] | (R)-1-(6-(5-(1-(3,5- dichloropyridin-4- yl)ethoxy)-1H- indazol-3- yl)pyridazin-3-yl)-3- (1-methyl-1H- pyrazol-3- yl)azetidin-3-amine | LCMS m/z = 536.2 (M + H); ¹HNMR (400 MHz, DMSO-d6) δ 13.18 (s, 1H), 8.56 (s, 2H), 7.98 (d, J = 9.6 Hz, 1H), 7.96 (d, J = 2.4 Hz, 1H), 7.65 (d, J = 1.6 Hz, 1H), 7.47 (J = 9.2 Hz, 1H), 7.09 (dd, J = 8.8 Hz, 2.4 Hz, 1H), 6.93 (d, J = 9.2 Hz, 1H), 6.36 (d, J = 2.0 Hz, 1H), 6.08 (q, J = 6.8 Hz, 1H), 4.41 (d, J = 8.0 Hz, 2H), 4.11 (d, J = 8.0 Hz, 2H), 3.83 (s, 3H), 1.77 (d, J = 6.8 Hz, 3H) |
| Example 389. | 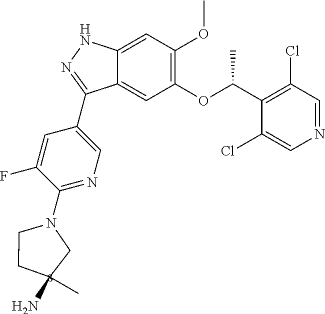 | (S)-1-(5-(5-((R)-1- (3,5-dichloropyridin- 4-yl)ethoxy)-6- methoxy-1H- indazol-3-yl)-3- fluoropyridin-2-yl)- 3-methylpyrrolidin- 3-amine | LCMS m/z = 531.3 (M + H); 1HNMR (400 MHz, DMSO-d6) δ 12.87 (s, 1H), 8.59 (s, 2H), 8.27 (t, J = 2.0 Hz, 1H), 7.61 (dd, J = 14.8 Hz, 2.0 Hz, 1H), 7.07 (s, 1H), 6.99 (s, 1H), 6.01 (q, J = 6.4 Hz, 1H), 3.86 (s, 3H), 3.82-3.76 (m, 1H), 3.72- 3.67 (m, 1H), 3.48-3.43 (m, 2H), 1.87 (brs, 2H), 1.81 (t, J = 6.8 Hz, 2H), 1.77 (d, J = 6.4 Hz, 3H), 1.27 (s, 3H) |
| Example 390. | 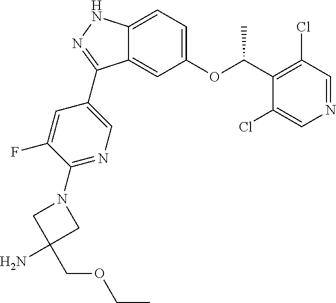 [SMILES: CCOCC1(N)CN(c2ncc(-c3n[nH]c4ccc(O[C@H](C)c5c(Cl)cncc5Cl)cc34)cc2F)C1] | 1-[5-[5-[(1R)-1-(3,5- dichloro-4- pyridyl)ethoxy]-1H- indazol-3-yl]-3- fluoro-2-pyridyl]-3- (ethoxymethyl)azeti- din-3-amine | LCMS m/z = 531.1 (M + H); 1HNMR (400 MHz, DMSO-d6) δ 8.96 (br s, 3H), 8.57 (s, 2H), 8.39 (s, 1H), 7.82 (dd, J = 1.4, 13.0 Hz, 1H), 7.49 (d, J = 9.0 Hz, 1H), 7.20 (d, J = 1.8 Hz, 1H), 7.10 (dd, J = 2.1, 9.0 Hz, 1H), 6.13 (q, J = 6.6 Hz, 1H), 4.32 (br d, J = 9.2 Hz, 2H), 4.22 (br d, J = 8.9 Hz, 2H), 3.85 (s, 2H), 3.61 (q, J = 7.0 Hz, 2H), 1.76 (d, J = 6.6 Hz, 3H), 1.19 (t, J = 7.0 Hz, 3H) |
| Example 391. | 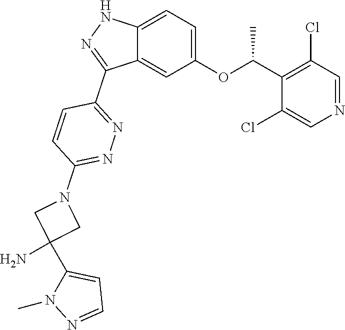 [SMILES: C[C@@H](Oc1ccc2[nH]nc(-c3ccc(N4CC(N)(c5ccnn5C)C4)nn3)c2c1)c1c(Cl)cncc1Cl] | (R)-1-(6-(5-(1-(3,5- dichloropyridin-4- yl)ethoxy)-1H- indazol-3- yl)pyridazin-3-yl)-3- (1-methyl-1H- pyrazol-5- yl)azetidin-3-amine | LCMS m/z = 536.2 (M + H); 1HNMR (400 MHz, DMSO-d6) δ 13.21 (s, 1H), 8.56 (s, 2H), 8.00 (d, J = 9.2 Hz, 1H), 7.94 (d, J = 2.0 Hz, 1H), 7.47 (d, J = 8.8 Hz, 1H), 7.36 (d, J = 2.0 Hz, 1H), 7.10 (dd, J = 9.2 Hz, 2.8 Hz, 1H), 6.95 (d, J = 9.6 Hz, 1H), 6.41 (d, J = 1.6 Hz, 1H), 6.08 (q, J = 6.8 Hz, 1H), 4.55 (dd, J = 8.4 Hz, 2.0 Hz, 2H), 4.25 (dd, J = 8.8 Hz, 2.0 Hz, 2H), 3.91 (s, 3H), 1.77 (d, J = 6.8 Hz, 3H) |
| Example 392. | 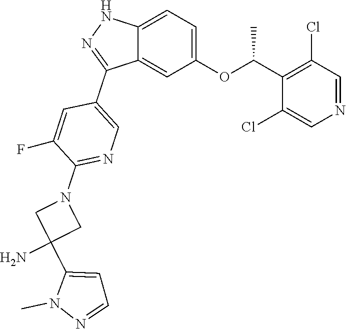 [SMILES: C[C@@H](Oc1ccc2[nH]nc(-c3cnc(N4CC(N)(c5ccnn5C)C4)c(F)c3)c2c1)c1c(Cl)cncc1Cl] | (R)-1-(5-(5-(1-(3,5- dichloropyridin-4- yl)ethoxy)-1H- indazol-3-yl)-3- fluoropyridin-2-yl)- 3-(1-methyl-1H- pyrazol-5- yl)azetidin-3-amine | LCMS m/z = 553.3 (M + H); 1HNMR (400 MHz, DMSO-d6) δ 13.13 (s, 1H), 8.57 (s, 2H), 8.39 (s, 1H), 7.73 (dd, J = 13.2 Hz, 1.6 Hz, 1H), 7.48 (d, J = 9.2 Hz, 1H), 7.34 (d, J = 1.6 Hz, 1H), 7.19 (s, 1H), 7.10 (dd, J = 9.2 Hz, 2.4 Hz, 1H), 6.41 (d, J = 2.0 Hz, 1H), 6.14 (q, J = 6.8 Hz, 1H), 4.52 (d, J = 7.2 Hz, 2H), 4.26 (d, J = 8.0 Hz, 2H), 3.90 (s, 3H), 2.74 (brs, 2H), 1.76 (d, J = 6.4 Hz, 3H) |
| Example 393. | 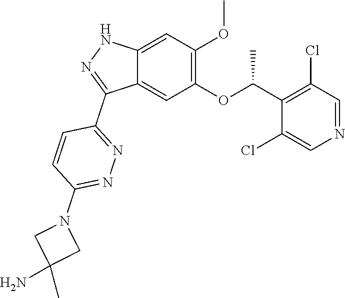 [SMILES: COc1cc2[nH]nc(-c3ccc(N4CC(C)(N)C4)nn3)c2cc1O[C@H](C)c1c(Cl)cncc1Cl] | (R)-1-(6-(5-(1-(3,5- dichloropyridin-4- yl)ethoxy)-6- methoxy-1H- indazol-3- yl)pyridazin-3-yl)-3- methylazetidin-3- amine | LCMS m/z = 500.2 (M + H); 1HNMR (400 MHz, DMSO-d6) δ 13.01 (s, 1H), 8.58 (s, 2H), 7.94 (d, J = 9.2 Hz, 1H), 7.83 (s, 1H), 7.01 (s, 1H), 6.87 (d, J = 9.2 Hz, 1H), 5.91 (q, J = 6.8 Hz, 1H), 3.98-3.93 (m, 2H), 3.89-3.84 (m, 5H), 1.75 (d, J = 6.8 Hz, 3H), 1.46 (s, 3H) |
| Example 394. | 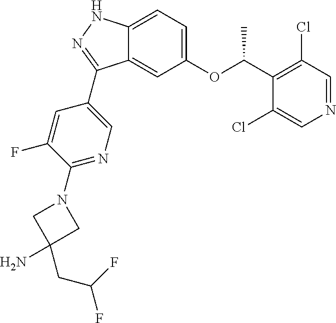 [SMILES: C[C@@H](Oc1ccc2[nH]nc(-c3cnc(N4CC(N)(CC(F)F)C4)c(F)c3)c2c1)c1c(Cl)cncc1Cl] | (R)-1-(5-(5-(1-(3,5- dichloropyridin-4- yl)ethoxy)-1H- indazol-3-yl)-3- fluoropyridin-2-yl)- 3-(2,2-difluoro- ethyl)azetidin- 3-amine | LCMS m/z = 537.2 (M + H); 1HNMR (400 MHz, DMSO-d6) δ 13.12 (s, 1H), 8.58 (s, 2H), 8.38 (t, J = 1.6 Hz, 1H), 7.71 (dd, J = 12.8 Hz, 1.4 Hz, 1H), 7.48 (d, J = 9.2 Hz, 1H), 7.20 (d, J = 2.0 Hz, 1H), 7.11 (dd, J = 9.2 Hz, 2.2 Hz, 1H), 6.44- 6.12 (m, 2H), 4.12 (d, J = 8.2 Hz, 2H), 3.92 (d, J = 8.0 Hz, 2H), 2.41-2.32 (m, 2H), 1.77 (d, J = 6.8 Hz, 3H) |
| Example 395. | 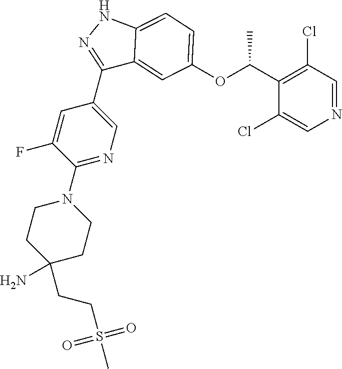 [SMILES: C[C@@H](Oc1ccc2[nH]nc(-c3cnc(N4CCC(N)(CCS(C)(=O)=O)CC4)c(F)c3)c2c1)c1c(Cl)cncc1Cl] | (R)-1-(5-(5-(1-(3,5- dichloropyridin-4- yl)ethoxy)-1H- indazol-3-yl)-3- fluoropyridin-2-yl)- 4-(2- (methylsulfonyl)ethyl) piperidin-4-amine | LCMS m/z = 607.3 (M + H); 1HNMR (400 MHz, DMSO-d6) δ 13.23 (s, 1H), 8.64 (s, 2H), 8.50 (d, J = 1.6 Hz, 1H), 7.81 (dd, J = 14.4 Hz, 1.6 Hz, 1H), 7.55 (d, J = 8.8 Hz, 1H), 7.26 (d, J = 2.4 Hz, 1H), 7.17 (dd, J = 9.2 Hz, 2.0 Hz, 1H), 6.08 (q, J = 6.8 Hz, 1H), 3.79-3.76 (m, 2H), 3.55-3.49 (m, 2H), 3.31- 3.27 (m, 2H), 3.05 (s, 3H), 1.88-1.81 (m, 5H), 1.72-1.65 (m, 2H), 1.62-1.56 (m, 2H) |
| Example 396. | 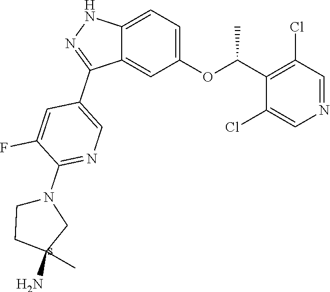 | (S)-1-(5-(5-((R)-1- (3,5-dichloropyridin- 4-yl)ethoxy)-1H- indazol-3-yl)-3- fluoropyridin-2-yl)- 3-methylpyrrolidin- 3-amine | LCMS m/z = 501.2 (M + H); 1HNMR (400 MHz, DMSO-d6) δ 13.07 (brs, 1H), 8.57 (s, 2H), 8.35 (s, 1H), 7.68 (dd, J = 14.4 Hz, 1.6 Hz, 1H), 7.47 (d, J = 8.8 Hz, 1H), 7.19 (d, J = 2.0 Hz, 1H), 7.11 (dd, J = 8.8 Hz, 2.0 Hz, 1H), 6.16 (q, J = 6.4 Hz, 1H), 3.84-3.77 (m, 1H), 3.74- 3.67 (m, 1H), 3.54-3.45 (m, 2H), 1.85 (t, J = 7.2 Hz, 2H), 1.77 (d, J = 6.4 Hz, 3H), 1.29 (s, 3H) |
| Example 397. | 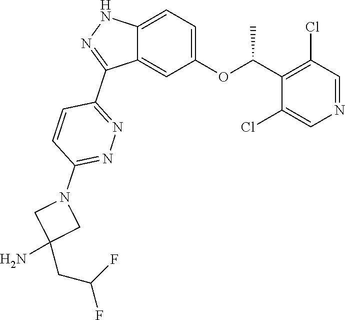 [SMILES: C[C@@H](Oc1ccc2[nH]nc(-c3ccc(N4CC(N)(CC(F)F)C4)nn3)c2c1)c1c(Cl)cncc1Cl] | (R)-1-(6-(5-(1-(3,5- dichloropyridin-4- yl)ethoxy)-1H- indazol-3- yl)pyridazin-3-yl)-3- (2,2-difluoro- ethyl)azetidin- 3-amine | LCMS m/z = 520.4 (M + H); 1HNMR (400 MHz, DMSO-d6) δ 13.17 (s, 1H), 8.55 (s, 2H), 7.98-7.94 (m, 2H), 7.47 (d, J = 8.8 Hz, 1H), 7.09 (dd, J = 9.2 Hz, 2.4 Hz, 1H), 6.90 (d, J = 9.2 Hz, 1H), 6.29 (tt, J = 56.8 Hz, 4.6 Hz, 1H), 6.08 (q, J = 6.6 Hz, 1H), 4.10 (d, J = 8.2 Hz, 2H), 3.85 (d, J = 8.0 Hz, 2H), 2.43- 2.30 (m, 2H), 1.77 (d, J = 6.6 Hz, 3H) |
| Example 398. | 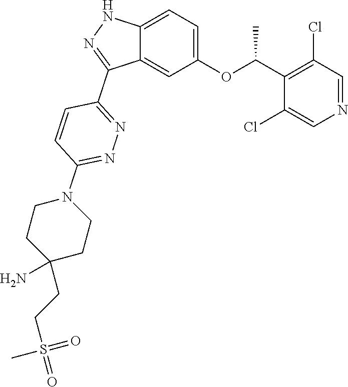 [SMILES: C[C@@H](Oc1ccc2[nH]nc(-c3ccc(N4CCC(N)(CCS(C)(=O)=O)CC4)nn3)c2c1)c1c(Cl)cncc1Cl] | (R)-1-(6-(5-(1-(3,5- dichloropyridin-4- yl)ethoxy)-1H- indazol-3- yl)pyridazin-3-yl)-4- (2- (methylsulfonyl)ethyl) piperidin-4-amine | LCMS m/z = 590.3 (M + H); 1HNMR (400 MHz, DMSO-d6) δ 13.17 (brs, 1H), 8.55 (s, 2H), 7.94-7.92 (m, 2H), 7.47 (d, J = 9.2 Hz, 1H), 7.35 (d, J = 9.2 Hz, 1H), 7.08 (dd, J = 9.2 Hz, 2.0 Hz, 1H), 6.06 (q, J = 6.4 Hz, 1H), 4.04-4.00 (m, 2H), 3.57- 3.51 (m, 2H), 3.25-3.20 (m, 2H), 2.97 (s, 3H), 1.80-1.76 (m, 5H), 1.60-1.49 (m, 4H) |
| Example 399. | 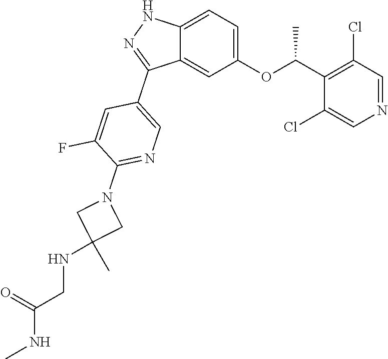 [SMILES: CNC(=O)CNC1(C)CN(c2ncc(-c3n[nH]c4ccc(O[C@H](C)c5c(Cl)cncc5Cl)cc34)cc2F)C1] | (R)-2-((1-(5-(5-(1- (3,5-dichloropyridin- 4-yl)ethoxy)-1H- indazol-3-yl)-3- fluoropyridin-2-yl)- 3-methylazetidin-3- yl)amino)-N- methylacetamide | LCMS m/z = 558.3 (M + H); 1HNMR (400 MHz, DMSO-d6) δ 13.11 (s, 1H), 8.57 (s, 2H), 8.37 (s, 1H), 7.83-7.81 (m, 1H), 7.71 (dd, J = 13.2 Hz, 1.6 Hz, 1H), 7.48 (d, J = 9.2 Hz, 1H), 7.20 (s, 1H), 7.11 (dd, J = 8.8 Hz, 2.0 Hz, 1H), 6.16 (q, J = 6.4 Hz, 1H), 3.40 (d, J = 8.0 Hz, 2H), 3.89 (d, J = 8.4 Hz, 2H), 3.16 (s, 2H), 2.64 (d, J = 4.4 Hz, 3H), 1.77 (d, J = 6.4 Hz, 3H), 1.43 (s, 3H) |
| Example 400. | 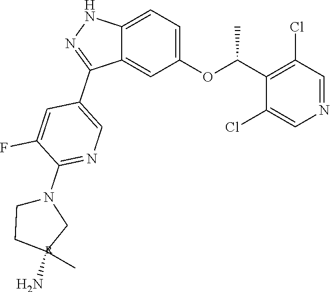 | (R)-1-(5-(5-((R)-1- (3,5-dichloropyridin- 4-yl)ethoxy)-1H- indazol-3-yl)-3- fluoropyridin-2-yl)- 3-methylpyrrolidin- 3-amine | LCMS m/z = 501.2 (M + H); 1HNMR (400 MHz, DMSO-d6) δ 13.08 (brs, 1H), 8.57 (s, 2H), 8.35 ( s, 1H), 7.67 (dd, J = 14.4 Hz, 1.6 Hz, 1H), 7.46 (d, J = 9.2 Hz, 1H), 7.19 (d, J = 2.0 Hz, 1H), 7.09 (dd, J = 9.2 Hz, 2.4 Hz, 1H), 6.14 (q, J = 6.8 Hz, 1H), 3.82-3.80 (m, 1H), 3.72- 3.71 (m, 1H), 3.58-3.55 (m, 1H), 3.49-3.47 (m, 1H), 1.89- 1.87 (m, 2H), 1.76 (d, J = 6.4 Hz, 3H), 1.31 (s, 3H) |
| Example 401. | 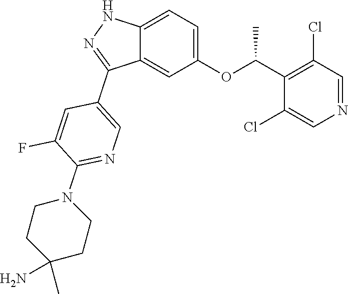 [SMILES: C[C@@H](Oc1ccc2[nH]nc(-c3cnc(N4CCC(C)(N)CC4)c(F)c3)c2c1)c1c(Cl)cncc1Cl] | fluoropyridin-2-yl)- 4-methylpiperidin-4- amine | LCMS m/z = 515.2 (M + H); 1HNMR (400 MHz, DMSO-d6) δ 13.17 (brs, 1H), 8.57 (s, 2H), 8.43 (s, 1H), 7.73 (dd, J = 14.4 Hz, 1.6 Hz, 1H), 7.48 (d, J = 9.2 Hz, 1H), 7.20 (d, J = 1.6 Hz, 1H), 7.10 (J = 8.8 Hz, 2.0 Hz, 1H), 6.14 (q, J = 6.8 Hz, 1H), 3.62-3.47 (m, 4H), 1.76 (d, J = 6.8 Hz, 3H), 1.60-1.54 (m, 2H), 1.52-1.46 (m, 2H), 1.12 (s, 3H) |
| Example 402. | 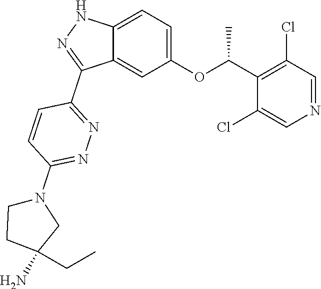 [SMILES: CC[C@@]1(N)CCN(c2ccc(-c3n[nH]c4ccc(O[C@H](C)c5c(Cl)cncc5Cl)cc34)nn2)C1] | (R)-1-(6-(5-((R)-1- (3,5-dichloropyridin- 4-yl)ethoxy)-1H- indazol-3- yl)pyridazin-3-yl)-3- ethylpyrrolidin-3- amine | LCMS m/z = 498.2 (M + H); 1HNMR (400 MHz, DMSO-d6) δ 13.14 (brs, 1H), 8.56 (d, J = 5.2 Hz, 2H), 7.96 (d, J = 1.6 Hz, 1H), 7.95 (d, J = 5.2 Hz, 1H), 7.46 (d, J = 8.8 Hz, 1H), 7.09 (dd, J = 9.2 Hz, 2.4 Hz, 1H), 6.96 (d, J = 9.2 Hz, 1H), 6.08 (q, J = 6.4 Hz, 1H), 3.70-3.66 (m, 2H), 3.57-3.55 (m, 1H), 3.46-3.43 (m, 1H), 2.02-1.94 (m, 2H), 1.77 (d, J = 6.8 Hz, 3H), 1.72-1.66 (m, 2H), 1.01 (t, J = 7.2 Hz, 3H) |
| Example 403. | 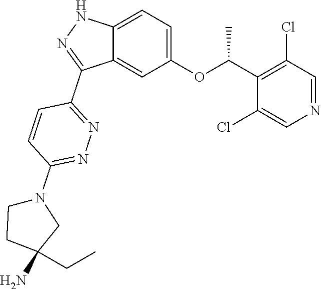 [SMILES: CC[C@]1(N)CCN(c2ccc(-c3n[nH]c4ccc(O[C@H](C)c5c(Cl)cncc5Cl)cc34)nn2)C1] | (S)-1-(6-(5-((R)-1- (3,5-dichloropyridin- 4-yl)ethoxy)-1H- indazol-3- yl)pyridazin-3-yl)-3- ethylpyrrolidin-3- amine | LCMS m/z = 498.2 (M + H); 1HNMR (400 MHz, DMSO-d6) δ 13.13 (brs, 1H), 8.56 d, J = 5.2 Hz, 2H), 7.96 (d, J = 2.8 Hz, 1H), 7.94 (d, J = 9.2 Hz, 1H), 7.46 (d, J = 8.8 Hz, 1H), 7.08 (dd, J = 8.8 Hz, 2.4 Hz, 1H), 6.94 (d, J = 9.6 Hz, 1H), 6.08 (q, J = 6.8 Hz, 1H), 3.69-3.65 (m, 2H), 3.48-3.46 (m, 1H), 3.41-3.38 (m, 1H), 1.98-1.89 (m, 2H), 1.77 (d, J = 7.2 Hz, 3H), 1.67-1.61 (m, 2H), 1.00 (t, J = 7.2 Hz, 3H) |
| Example 404. | 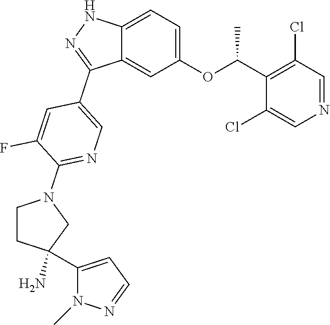 [SMILES: C[C@@H](Oc1ccc2[nH]nc(-c3cnc(N4CC[C@](N)(c5ccnn5C)C4)c(F)c3)c2c1)c1c(Cl)cncc1Cl] | (R)-1-(5-(5-((R)-1- (3,5-dichloropyridin- 4-yl)ethoxy)-1H- indazol-3-yl)-3- fluoropyridin-2-yl)- 3-(1-methyl-1H- pyrazol-5- yl)pyrrolidin-3- amine | LCMS m/z = 567.3 (M + H); 1HNMR (400 MHz, DMSO-d6) δ 13.10 (s, 1H), 8.58 (s, 2H), 8.40 (s, 1H), 7.75 (dd, J = 14.4 Hz, 1.2 Hz, 1H), 7.49 (d, J = 9.2 Hz, 1H), 7.35 (d, J = 1.2 Hz, 1H), 7.22 (s, 1H), 7.12 (dd, J = 9.2 Hz, 2.0 Hz, 1H), 6.26 (s, 1H), 6.16 (q, J = 6.4 Hz, 1H), 4.16-4.05 (m, 4H), 3.98-3.92 (m, 2H), 3.81-3.77 (m, 1H), 2.45-2.42 (m, 2H), 1.77 (d, J = 6.8 Hz, 3H) |
| Example 405. | 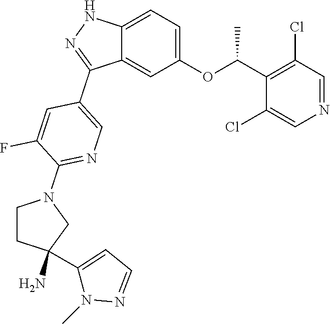 [SMILES: C[C@@H](Oc1ccc2[nH]nc(-c3cnc(N4CC[C@@](N)(c5ccnn5C)C4)c(F)c3)c2c1)c1c(Cl)cncc1Cl] | (S)-1-(5-(5-((R)-1- (3,5-dichloropyridin- 4-yl)ethoxy)-1H- indazol-3-yl)-3- fluoropyridin-2-yl)- 3-(1-methyl-1H- pyrazol-5- yl)pyrrolidin-3- amine | LCMS m/z = 567.3 (M + H); 1HNMR (400 MHz, DMSO-d6) δ 13.08 (s, 1H), 8.58 (s, 2H), 8.39 (s, 1H), 7.72 (dd, J = 14.4 Hz, 2.0 Hz, 1H), 7.48 (d, J = 8.8 Hz, 1H), 7.30 (d, J = 1.6 Hz, 1H), 7.22-7.21 (m, 1H), 7.11 (dd, J = 9.2 Hz, 2.0 Hz, 1H), 6.18-6.12 (m, 2H), 4.05 (s, 3H), 3.99-3.87 (m, 3H), 3.77-3.71 (m, 1H), 2.40-2.25 (m, 2H), 1.77 (d, J = 6.4 Hz, 3H) |
| Example 406. | 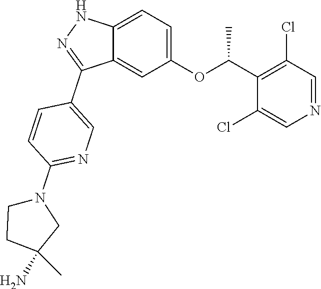 [SMILES: C[C@@H](Oc1ccc2[nH]nc(-c3ccc(N4CC[C@@](C)(N)C4)nc3)c2c1)c1c(Cl)cncc1Cl] | (R)-1-(6-(5-((R)-1- (3,5-dichloropyridin- 4-yl)ethoxy)-1H- indazol-3- yl)pyridazin-3-yl)-3- methylpyrrolidin-3- amine | LCMS m/z = 484.2 (M + H); 1HNMR (400 MHz, DMSO-d6) δ 13.13 (s, 1H), 8.56 (s, 2H), 7.96-7.93 (m, 2H), 7.46 (d, J = 8.8 Hz, 1H), 7.08 (dd, J = 9.2 Hz, 2.4Hz, 1H), 6.92 (d, J = 9.6 Hz, 1H), 6.08 (q, J = 6.4 Hz, 1H), 3.70-3.60 (m, 2H), 3.50- 3.39 (m, 2H), 1.97-1.93 (m, 2H), 1.77 (d, J = 6.4 Hz, 3H), 1.34 (s, 3H) |
| Example 407. | 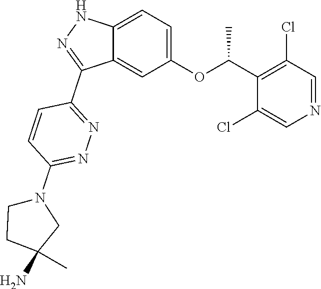 [SMILES: C[C@@H](Oc1ccc2[nH]nc(-c3ccc(N4CC[C@](C)(N)C4)nn3)c2c1)c1c(Cl)cncc1Cl] | (S)-1-(6-(5-((R)-1- (3,5-dichloropyridin- 4-yl)ethoxy)-1H- indazol-3- yl)pyridazin-3-yl)-3- methylpyrrolidin-3- amine | LCMS m/z = 484.2 (M + H); 1HNMR (400 MHz, DMSO-d6) δ 13.11 (brs, 1H), 8.56 (s, 2H), 7.96 (d, J = 2.4 Hz, 1H), 7.94 (d, J = 9.6 Hz, 1H), 7.47 (d, J = 8.8 Hz, 1H), 7.10 (dd, J = 8.8 Hz, 2.4 Hz, 1H), 6.92 (d, J = 9.2 Hz, 1H), 6.10 (q, J = 6.8 Hz, 1H), 3.72-3.67 (m, 1H), 3.63- 3.59 (m, 1H), 3.42-3.36 (m, 2H), 2.21 (brs, 2H), 1.92 (t, J = 6.8 Hz, 2H), 1.78 (d, J = 6.8 Hz, 3H), 1.31 (s, 3H) |
| Example 408. | 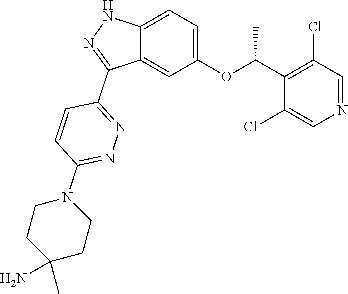 | (R)-1-(5-(5-((R)-1- (3,5-dichloropyridin- 4-yl)ethoxy)-1H- indazol-3-yl)-3- fluoropyridin-2-yl)- 3-methylpyrrolidin- 3-amine | LCMS m/z = 501.2 (M + H); 1HNMR (400 MHz, DMSO-d6) δ 13.08 (brs, 1H), 8.57 (s, 2H), 8.35 ( s, 1H), 7.67 (dd, J = 14.4 Hz, 1.6 Hz, 1H), 7.46 (d, J = 9.2 Hz, 1H), 7.19 (d, J = 2.0 Hz, 1H), 7.09 (dd, J = 9.2 Hz, 2.4 Hz, 1H), 6.14 (q, J = 6.8 Hz, 1H), 3.82-3.80 (m, 1H), 3.72- 3.71 (m, 1H), 3.58-3.55 (m, 1H), 3.49-3.47 (m, 1H), 1.89- 1.87 (m, 2H), 1.76 (d, J = 6.4 Hz, 3H), 1.31 (s, 3H) |
| Example 409. | 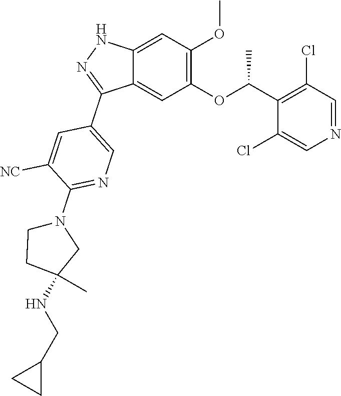 [SMILES: COc1cc2[nH]nc(-c3cnc(N4CC[C@@](C)(NCC5CC5)C4)c(C#N)c3)c2cc1O[C@H](C)c1c(Cl)cncc1Cl] | 2-((R)-3-((cyclo- propylmethyl)- amino)-3- methylpyrrolidin-1- yl)-5-(5-((R)-1-(3,5- dichloropyridin-4- yl)ethoxy)-6- methoxy-1H- indazol-3- yl)nicotinonitrile | LCMS m/z = 592.3 (M + H); 1HNMR (400 MHz, DMSO-d6) δ 12.95 (s, 1H), 8.72 (d, J = 2.4 Hz, 1H), 8.59 (s, 2H), 8.04 (d, J = 2.4 Hz, 1H), 7.03-7.00 (m, 2H), 6.00 (q, J = 6.8 Hz, 1H), 3.91-3.73 (m, 6H), 3.56 (d, J = 10.8 Hz, 1H), 2.48-2.45 (m, 2H), 2.08-2.03 (m, 1H), 1.87- 1.82 (m, 1H), 1.76 (d, J = 6.8 Hz, 3H), 1.26 (s, 3H), 0.90-0.85 (m, 1H), 0.43-0.41 (m, 2H), 0.15-0.14 (m, 2H) |
| Example 410. | 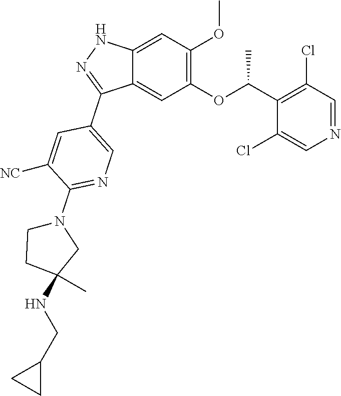 [SMILES: COc1cc2[nH]nc(-c3cnc(N4CC[C@](C)(NCC5CC5)C4)c(C#N)c3)c2cc1O[C@H](C)c1c(Cl)cncc1Cl] | 2-((S)-3- ((cyclopropyl- methyl)- amino)-3- methylpyrrolidin-1- yl)-5-(5-((R)-1-(3,5- dichloropyridin-4- yl)ethoxy)-6- methoxy-1H- indazol-3- yl)nicotinonitrile | LCMS m/z = 592.3 (M + H); 1HNMR (400 MHz, DMSO-d6) δ 12.95 (s, 1H), 8.72 (d, J = 2.4 Hz, 1H), 8.60 (s, 2H), 8.04 (d, J = 2.0 Hz, 1H), 7.03 (s, 1H), 7.01 (s, 1H), 6.00 (q, J = 6.8 Hz, 1H), 3.94-3.90 (m, 1H), 3.87 (s, 3H), 3.84-3.72 (m, 2H), 3.61-3.54 (m, 1H), 2.47-2.40 (m, 2H), 2.10-1.98 (m, 1H), 1.90-1.84 (m, 1H), 1.76 (d, J = 6.4 Hz, 3H), 1.26 (s, 3H), 0.89- 0.86 (m, 1H), 0.43-0.41 (m, 2H), 0.20-0.10 (m, 2H) |
| Example 411. | 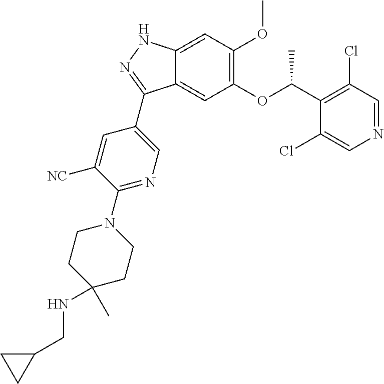 [SMILES: COc1cc2[nH]nc(-c3cnc(N4CCC(C)(NCC5CC5)CC4)c(C#N)c3)c2cc1O[C@H](C)c1c(Cl)cncc1Cl] | (R)-2-(4-((cyclo- propylmethyl) amino)-4- methylpiperidin-1- yl)-5-(5-(1-(3,5- dichloropyridin-4- yl)ethoxy)-6- methoxy-1H- indazol-3- yl)nicotinonitrile | LCMS m/z = 606.5 (M + H); 1HNMR (400 MHz, DMSO-d6) δ 13.01 (brs, 1H), 8.76 (d, J = 2.4 Hz, 1H), 8.59 (s, 2H), 8.14 (d, J = 2.0 Hz, 1H), 7.06 (s, 1H), 7.01 (s, 1H), 6.01 (q, J = 6.8 Hz, 1H), 3.86 (s, 3H), 3.76- 3.68 (m, 4H), 2.46-2.37 (m, 2H), 1.76 (d, J = 6.4 Hz, 3H), 1.75-1.67 (m, 2H), 1.57-1.55 (m, 2H), 1.11 (s, 3H), 0.94-0.83 (m, 1H), 0.44-0.41 (m, 2H), 0.19-0.11 (m, 2H) |
| Example 412. | 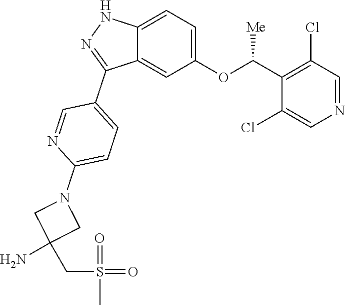 | 1-[5-[5-[(1R)-1-(3,5- dichloro-4- pyridyl)ethoxy]-1H- indazol-3-yl]-3- fluoro-2-pyridyl]-3- (methylsulfonyl- methyl)azetidin-3- amine | LCMS m/z = 565 (M + H); 1HNMR (400 MHz, DMSO-d6) δ 9.09 (br s, 3H), 8.57 (s, 2H), 8.43 (s, 1H), 7.82 (dd, J = 1.7, 13.0 Hz, 1H), 7.49 (d, J = 9.0 Hz, 1H), 7.20 (d, J = 2.1 Hz, 1H), 7.11 (dd, J = 2.2, 9.0 Hz, 1H), 6.14 (q, J = 6.6 Hz, 1H), 4.52 (br d, J = 9.3 Hz, 2H), 4.37 (br d, J = 9.2 Hz, 2H), 4.12 (s, 2H), 3.23 (s, 3H), 1.76 (d, J = 6.6 Hz, 3H) |
| Example 413. | 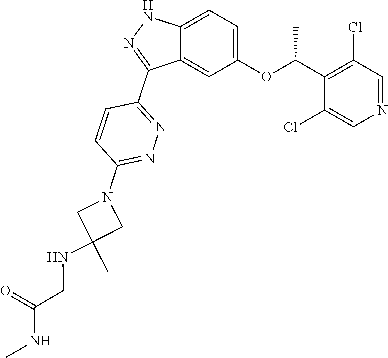 [SMILES: CNC(=O)CNC1(C)CN(c2ccc(-c3n[nH]c4ccc(O[C@H](C)c5c(Cl)cncc5Cl)cc34)nn2)C1] | (R)-2-((1-(6-(5-(1- (3,5-dichloropyridin- 4-yl)ethoxy)-1H- indazol-3- yl)pyridazin-3-yl)-3- methylazetidin-3- yl)amino)-N- methylacetamide | LCMS m/z = 541.2 (M + H); 1HNMR (400 MHz, DMSO-d6) δ 13.23 (s, 1H), 8.56 (s, 2H), 8.17 (brs, 1H), 8.02 (d, J = 9.2 Hz, 1H), 7.94 (d, J = 2.0 Hz, 1H), 7.49 (d, J = 9.2 Hz, 1H), 7.12 (dd, J = 9.2 Hz, 2.4 Hz, 1H), 6.96 (d, J = 9.2 Hz, 1H), 6.11 (q, J = 6.4 Hz, 1H), 4.18 (d, J = 8.0 Hz, 2H), 4.00 (d, J = 8.8 Hz, 2H), 3.54 (s, 2H), 2.68 (d, J = 4.8 Hz, 3H), 1.78 (d, J = 6.8 Hz, 3H), 1.58 (s, 3H) |
| Example 414. | 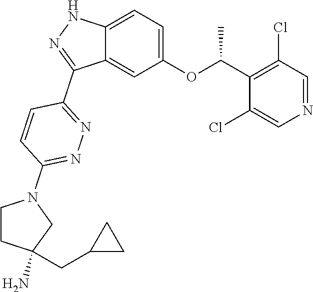 [SMILES: C[C@@H](Oc1ccc2[nH]nc(-c3ccc(N4CC[C@](N)(CC5CC5)C4)nn3)c2c1)c1c(Cl)cncc1Cl] | (R)-3-(cyclopropyl- methyl)-1-(6-(5- ((R)-1-(3,5- dichloropyridin-4- yl)ethoxy)-1H- indazol-3- yl)pyridazin-3- yl)pyrrolidin-3- amine | LCMS m/z = 524.3 (M + H); 1HNMR (400 MHz, DMSO-d6) δ 13.14 (brs, 1H), 8.56 (s, 2H), 7.98-7.93 (m, 2H), 7.47 (d, J = 8.8 Hz, 1H), 7.09 (dd, J = 8.8 Hz, 2.4 Hz, 1H), 6.98 (d, J = 9.6 Hz, 1H), 6.08 (q, J = 6.4 Hz, 1H), 3.68-3.54 (m, 4H), 2.14- 2.02 (m, 2H), 1.78 (d, J = 6.8 Hz, 3H), 1.66-1.56 (m, 2H), 0.93-0.83 (m, 1H), 0.52-0.50 (m, 2H), 0.17-0.15 (m, 2H) |
| Example 415. | 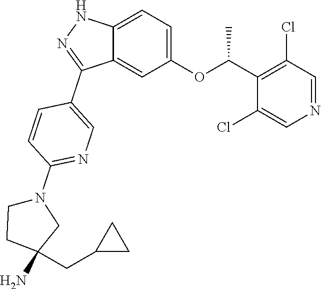 [SMILES: C[C@@H](Oc1ccc2[nH]nc(-c3ccc(N4CC[C@@](N)(CC5CC5)C4)nc3)c2c1)c1c(Cl)cncc1Cl] | (S)-3-(cyclopropyl- methyl)-1-(6-(5- ((R)-1-(3,5- dichloropyridin-4- yl)ethoxy)-1H- indazol-3- yl)pyridazin-3- yl)pyrrolidin-3- amine | LCMS m/z = 524.2 (M + H); 1HNMR (400 MHz, DMSO-d6) δ 13.12 (brs, 1H), 8.56 (s, 2H), 7.97-7.93 (m, 2H), 7.46 (d, J = 9.2 Hz, 1H), 7.08 (dd, J = 8.8 Hz, 2.4 Hz, 1H), 6.94 (d, J = 9.2 Hz, 1H), 6.08 (q, J = 6.4 Hz, 1H), 3.69-3.65 (m, 2H), 3.65- 3.50 (m, 2H), 2.08-1.94 (m, 2H), 1.78 (d, J = 6.8 Hz, 3H), 1.59-1.50 (m, 2H), 0.92-0.85 (m, 1H), 0.52-0.48 (m, 2H), 0.15-0.12 (m, 2H) |
| Example 416. | 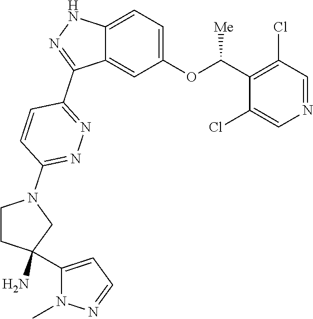 [SMILES: C[C@@H](Oc1ccc2[nH]nc(-c3ccc(N4CC[C@@](N)(c5ccnn5C)C4)nn3)c2c1)c1c(Cl)cncc1Cl] | (R)-1-(6-(5-((R)-1- (3,5-dichloropyridin- 4-yl)ethoxy)-1H- indazol-3- yl)pyridazin-3-yl)-3- (1-methyl-1H- pyrazol-5- yl)pyrrolidin-3- amine | LCMS m/z = 550.3 (M + H); 1HNMR (400 MHz, DMSO-d6) δ 13.15 (s, 1H), 8.56 (s, 2H), 8.00-7.98 (m, 2H), 7.48 (d, J = 9.2 Hz, 1H), 7.34 (d, J = 1.6 Hz, 1H), 7.10-7.05 (m, 2H), 6.27 (s, 1H), 6.09 (q, J = 6.4 Hz, 1H), 4.08-4.04 (m, 4H), 3.93 (d, J = 11.2 Hz, 1H), 3.80-3.70 (m, 2H), 2.49-2.44 (m, 2H), 1.78 (d, J = 6.8 Hz, 3H) |
| Example 417. | 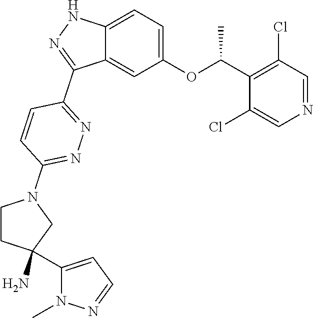 [SMILES: C[C@@H](Oc1ccc2[nH]nc(-c3ccc(N4CC[C@@](N)(c5ccnn5C)C4)nn3)c2c1)c1c(Cl)cncc1Cl] | (S)-1-(6-(5-((R)-1- (3,5-dichloropyridin- 4-yl)ethoxy)-1H- indazol-3- yl)pyridazin-3-yl)-3- (1-methyl-1H- pyrazol-5- yl)pyrrolidin-3- amine | LCMS m/z = 550.2 (M + H); 1HNMR (400 MHz, DMSO-d6) δ 13.15 (s, 1H), 8.56 (s, 2H), 8.00-7.98 (m, 2H), 7.48 (d, J = 9.2 Hz, 1H), 7.35 (d, J = 1.6 Hz, 1H), 7.10-7.05 (m, 2H), 6.27 (s, 1H), 6.09 (q, J = 6.8 Hz, 1H), 4.08-4.04 (m, 4H), 3.93 (d, J = 11.2 Hz, 1H), 3.80-3.70 (m, 2H), 2.56-2.52 (m, 2H), 1.78 (d, J = 6.4 Hz, 3H) |
| Example 418. | 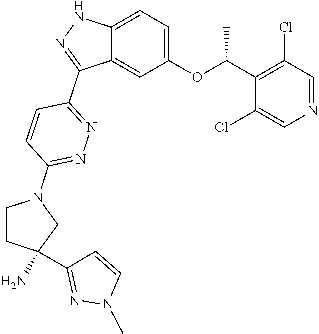 [SMILES: C[C@@H](Oc1ccc2[nH]nc(-c3ccc(N4CC[C@](N)(c5ccn(C)n5)C4)nn3)c2c1)c1c(Cl)cncc1Cl] | (R)-1-(6-(5-((R)-1- (3,5-dichloropyridin- 4-yl)ethoxy)-1H- indazol-3- yl)pyridazin-3-yl)-3- (1-methyl-1H- pyrazol-3- yl)pyrrolidin-3- amine | LCMS m/z = 550.3 (M + H); 1HNMR (400 MHz, DMSO-d6) δ 13.16 (s, 1H), 8.55 (s, 2H), 8.00 (d, J = 9.6 Hz, 1H), 7.95 (d, J = 2.4 Hz, 1H), 7.76 (d, J = 2.0 Hz, 1H), 7.48 (d, J = 9.2 Hz, 1H), 7.11 (dd, J = 8.8 Hz, 2.4 Hz, 1H), 7.05 (d, J = 9.6 Hz, 1H), 6.48 (d, J = 2.4 Hz, 1H), 6.11 (q, J = 6.8 Hz, 1H), 4.03- 3.95 (m, 2H), 3.87 (s, 3H), 3.83-3.73 (m, 2H), 2.63-2.55 (m, 1H), 2.40-2.38 (m, 1H), 1.78 (d, J = 6.8 Hz, 3H) |
| Example 419. | 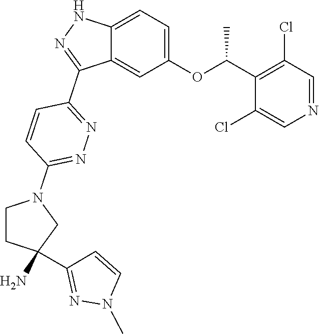 [SMILES: C[C@@H](Oc1ccc2[nH]nc(-c3ccc(N4CC[C@@](N)(c5ccn(C)n5)C4)nn3)c2c1)c1c(Cl)cncc1Cl] | (S)-1-(6-(5-((R)-1- (3,5-dichloropyridin- 4-yl)ethoxy)-1H- indazol-3- yl)pyridazin-3-yl)-3- (1-methyl-1H- pyrazol-3- yl)pyrrolidin-3- amine | LCMS m/z = 550.3 (M + H); 1HNMR (400 MHz, DMSO-d6) δ 13.13 (s, 1H), 8.56 (s, 2H), 7.97-7.94 (m, 2H), 7.67 (d, J = 2.4 Hz, 1H), 7.47 (d, J = 8.8 Hz, 1H), 7.10 (dd, J = 9.2 Hz, 2.4 Hz, 1H), 6.99 (d, J = 9.6 Hz, 1H), 6.39 (d, J = 2.4 Hz, 1H), 6.11 (q, J = 6.8 Hz, 1H), 3.91- 3.88 (m, 1H), 3.83 (s, 3H), 3.79-3.71 (m, 3H), 2.47-2.43 (m, 1H), 2.21-2.19 (m, 1H), 1.78 (d, J = 6.4 Hz, 3H) |
| Example 420. | 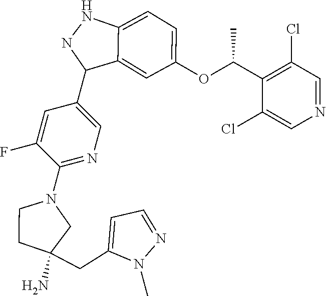 [SMILES: C[C@@H](Oc1ccc2c(c1)C(c1cnc(N3CC[C@](N)(Cc4ccnn4C)C3)c(F)c1)NN2)c1c(Cl)cncc1Cl] | (S)-1-(5-(5-((R)-1- (3,5-dichloropyridin- 4-yl)ethoxy)-1H- indazol-3-yl)-3- fluoropyridin-2-yl)- 3-((1-methyl-1H- pyrazol-5-yl) methyl)pyrrolidin- 3-amine | LCMS m/z = 581.3 (M + H); 1HNMR (400 MHz, DMSO-d6) δ 13.07 (s, 1H), 8.58 (d, J = 8.0 Hz, 2H), 8.36 (t, J = 1.6 Hz, 1H), 7.70 (d, J = 1.6 Hz, 1H), 7.47 (d, J = 9.2 Hz, 1H), 7.36 (d, J = 0.8 Hz, 1H), 7.20 (d, J = 2.0 Hz, 1H), 7.09 (d, J = 9.2 Hz, 1H), 6.19 (d, J = 2.0 Hz, 1H), 6.14 (q, J = 6.4 Hz, 1H), 3.82 (s, 3H), 3.79-3.71 (m, 2H), 3.63-3.52 (m, 2H), 3.00 (d, J = 2.4 Hz, 2H), 2.01-1.94 (m, 1H), 1.87-1.83 (m, 1H), 1.76 (d, J = 6.8 Hz, 3H) |
| Example 421. | 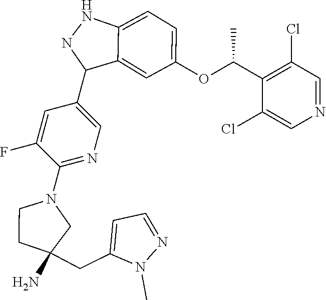 [SMILES: C[C@@H](Oc1ccc2c(c1)C(c1cnc(N3CC[C@@](N)(Cc4ccnn4C)C3)c(F)c1)NN2)c1c(Cl)cncc1Cl] | (R)-1-(5-(5-((R)-1- (3,5-dichloropyridin- 4-yl)ethoxy)-1H- indazol-3-yl)-3- fluoropyridin-2-yl)- 3-((1-methyl-1H- pyrazol-5-yl) methyl)pyrrolidin- 3-amine | LCMS m/z = 581.3 (M + H); 1HNMR (400 MHz, DMSO-d6) δ 13.08 (s, 1H), 8.57 (d, J = 4.4 Hz, 2H), 8.37 (d, J = 6.0 Hz, 1H), 7.69 (dd, J = 14.0 Hz, 1.6 Hz, 1H), 7.47 (d, J = 8.8 Hz, 1H), 7.40-7.38 (m, 1H), 7.21- 7.19 (m, 1H), 7.10 (dd, J = 9.2 Hz, 2.0 Hz, 1H), 6.23 (d, J = 6.8 Hz, 1H), 6.14 (q, J = 6.0 Hz, 1H), 3.82 (s, 3H), 3.80-3.76 (m, 2H), 3.66-3.64 (m, 2H), 3.07 (s, 2H), 2.05-2.02 (m, 1H), 1.95- 1.90 (m, 1H), 1.76 (d, J = 6.4 Hz, 3H) |
| Example 422. | 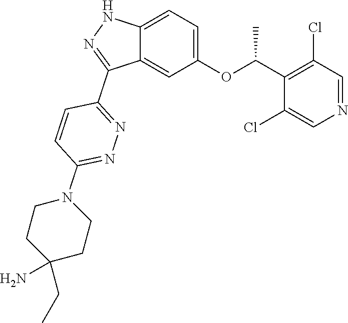 [SMILES: CCC1(N)CCN(c2ccc(-c3n[nH]c4ccc(O[C@H](C)c5c(Cl)cncc5Cl)cc34)nn2)CC1] | (R)-1-(6-(5-(1-(3,5- dichloropyridin-4- yl)ethoxy)-1H- indazol-3- yl)pyridazin-3-yl)-4- ethylpiperidin-4- amine | LCMS m/z = 512.3 (M + H); 1HNMR (400 MHz, DMSO-d6) δ 13.17 (brs, 1H), 8.56 (s, 2H), 7.95-7.92 (m, 2H), 7.47 (d, J = 8.8 Hz, 1H), 7.35 (d, J = 9.6 Hz, 1H), 7.09 (dd, J = 8.8 Hz, 2.0 Hz, 1H), 6.07 (q, J = 7.2 Hz, 1H), 3.97-3.93 (m, 2H), 3.63- 3.56 (m, 2H), 1.77 (d, J = 6.8 Hz, 3H), 1.59-1.42 (m, 6H), 0.88 (t, J = 7.2 Hz, 3H) |
| Example 423. | 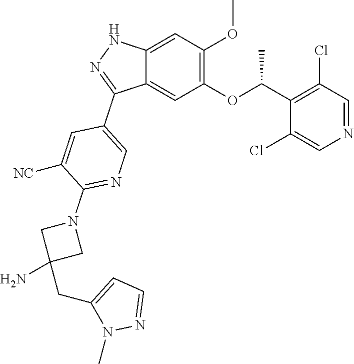 [SMILES: COc1cc2[nH]nc(-c3cnc(N4CC(N)(Cc5ccnn5C)C4)c(C#N)c3)c2cc1O[C@H](C)c1c(Cl)cncc1Cl] | (R)-2-(3-amino-3- ((1-methyl-1H- pyrazol-5- yl)methyl)azetidin- 1-yl)-5-(5-(1-(3,5- dichloropyridin-4- yl)ethoxy)-6- methoxy-1H- indazol-3- yl)nicotinonitrile | LCMS m/z = 604.3 (M + H); 1HNMR (400 MHz, DMSO-d6) δ 1H NMR (400 MHZ, DMSO- d6) 8 12.97 (s, 1H), 8.72 (d, J = 2.8 Hz, 1H), 8.58 (s, 2H), 8.08 (d, J = 1.6 Hz, 1H), 7.34 (d, J = 1.6 Hz, 1H), 7.03 (s, 1H), 7.01 (s, 1H), 6.23 (d, J = 1.6 Hz, 1H), 5.99 (q, J = 6.8 Hz, 1H), 4.30 (d, J = 8.8 Hz, 2H), 4.12 (d, J = 9.2 Hz, 2H), 3.86 (s, 3H), 3.81 (s, 3H), 3.17 (s, 2H), 1.76 (d, J = 6.8 Hz, 3H) |
| Example 424. | 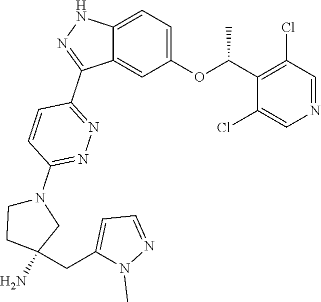 [SMILES: C[C@@H](Oc1ccc2[nH]nc(-c3ccc(N4CC[C@](N)(Cc5ccnn5C)C4)nn3)c2c1)c1c(Cl)cncc1Cl] | (S)-1-(6-(5-((R)-1- (3,5-dichloropyridin- 4-yl)ethoxy)-1H- indazol-3- yl)pyridazin-3-yl)-3- ((1-methyl-1H- pyrazol-5-yl) methyl)pyrrolidin- 3-amine | LCMS m/z = 564.3 (M + H); 1HNMR (400 MHz, DMSO-d6) δ 1H NMR (400 MHz, DMSO- d6) δ 13.11 (s, 1H), 8.56 (s, 2H), 7.96 (d, J = 2.0 Hz, 1H), 7.93 (d, J = 9.2 Hz, 1H), 7.45 (d, J = 9.2 Hz, 1H), 7.36 (d, J = 2.0 Hz, 1H), 7.08 (dd, J = 9.2 Hz, 2.4 Hz, 1H), 6.93 (d, J = 9.2 Hz, 1H), 6.21 (d, J = 1.6 Hz, 1H), 6.08 (q, J = 6.4 Hz, 1H), 3.83 (s, 3H), 3.71-3.65 (m, 2H), 3.55-3.52 (m, 1H), 3.46-3.40 (m, 1H), 3.00 (s, 2H), 2.08-2.00 (m, 1H), 1.92-1.87 (m, 1H), 1.77 (d, J = 6.8 Hz, 3H) |
| Example 425. | 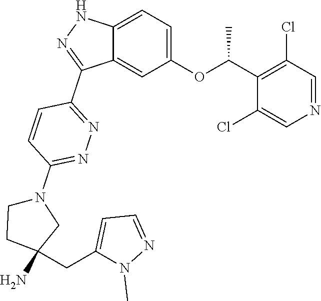 [SMILES: C[C@@H](Oc1ccc2[nH]nc(-c3ccc(N4CC[C@@](N)(Cc5ccnn5C)C4)nn3)c2c1)c1c(Cl)cncc1Cl] | (R)-1-(6-(5-((R)-1- (3,5-dichloropyridin- 4-yl)ethoxy)-1H- indazol-3- yl)pyridazin-3-yl)-3- ((1-methyl-1H- pyrazol-5-yl) methyl)pyrrolidin- 3-amine | LCMS m/z = 564.3 (M + H); 1HNMR (400 MHz, DMSO-d6) δ 1H NMR (400 MHz, DMSO- d6) δ 13.11 (s, 1H), 8.55 (s, 2H), 7.96 (d, J = 2.0 Hz, 1H), 7.93 (d, J = 9.2 Hz, 1H), 7.45 (d, J = 9.2 Hz, 1H), 7.36 (d, J = 2.0 Hz, 1H), 7.08 (dd, J = 8.8 Hz, 2.4 Hz, 1H), 6.93 (d, J = 9.6 Hz, 1H), 6.21 (d, J = 1.6 Hz, 1H), 6.08 (q, J = 6.8 Hz, 1H), 3.83 (s, 3H), 3.71-3.65 (m, 2H), 3.54-3.51 (m, 1H), 3.45-3.42 (m, 1H), 3.00 (s, 2H), 2.07-2.00 (m, 1H), 1.91-1.86 (m, 1H), 1.77 (d, J = 6.8 Hz, 3H) |
| Example 426. | 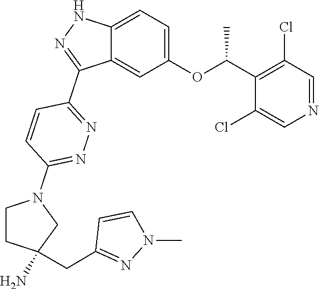 [SMILES: C[C@@H](Oc1ccc2[nH]nc(-c3ccc(N4CC[C@](N)(Cc5ccn(C)n5)C4)nn3)c2c1)c1c(Cl)cncc1Cl] | (S)-1-(6-(5-((R)-1- (3,5-dichloropyridin- 4-yl)ethoxy)-1H- indazol-3- yl)pyridazin-3-yl)-3- ((1-methyl-1H- pyrazol-3-yl) methyl)pyrrolidin- 3-amine | LCMS m/z = 564.3 (M + H); 1HNMR (400 MHz, DMSO-d6) δ 1H NMR (400 MHz, DMSO- d6) δ 13.11 (s, 1H), 8.56 (d, J = 5.2 Hz, 2H), 7.96 (d, J = 2.0 Hz, 1H), 7.94 (d, J = 9.6 Hz, 1H), 7.62 (d, J = 1.2 Hz, 1H), 7.46 (d, J = 8.8 Hz, 1H), 7.08 (dd, J = 8.8 Hz, 2.4 Hz, 1H), 6.91 (d, J = 9.6 Hz, 1H), 6.16 (d, J =2.0 Hz, 1H), 6.08 (q, J = 7.2 Hz, 1H), 3.82 (s, 3H), 3.69-3.57 (m, 4H), 2.89 (s, 2H), 2.11-2.05 (m, 1H), 1.94-1.87 (m, 1H), 1.77 (d, J = 6.4 Hz, 3H) |
| Example 427. | 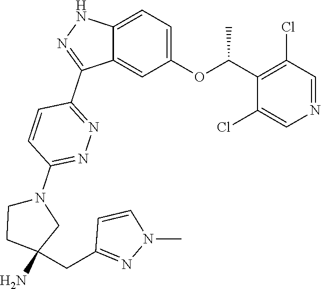 | (R)-1-(6-(5-((R)-1- (3,5-dichloropyridin- 4-yl)ethoxy)-1H- indazol-3- yl)pyridazin-3-yl)-3- ((1-methyl-1H- pyrazol-3-yl) methyl)pyrrolidin- 3-amine | LCMS m/z = 5641.4 (M + H); 1HNMR (400 MHz, DMSO-d6) δ 1H NMR (400 MHz, DMSO- d6) δ 13.12 (s, 1H), 8.56 (d, J = 5.2 Hz, 2H), 7.96 (d, J = 2.0 Hz, 1H), 7.93 (d, J = 9.6 Hz, 1H), 7.61 (d, J = 1.6 Hz, 1H), 7.45 (d, J = 9.2 Hz, 1H), 7.08 (dd, J = 9.2 Hz, 2.4 Hz, 1H), 6.90 (d, J = 9.2 Hz, 1H), 6.15 (d, J = 2.0 Hz, 1H), 6.08 (q, J = 6.4 Hz, 1H), 3.81 (s, 3H), 3.66-3.54 (m, 4H), 2.86 (s, 2H), 2.08-2.01 (m, 1H), 1.89-1.83 (m, 1H), 1.77 (d, J = 6.8 Hz, 3H) |
| Example 428. | 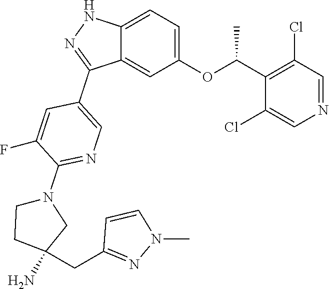 [SMILES: C[C@@H](Oc1ccc2[nH]nc(-c3cnc(N4CC[C@](N)(Cc5ccn(C)n5)C4)c(F)c3)c2c1)c1c(Cl)cncc1Cl] | (S)-1-(5-(5-((R)-1- (3,5-dichloropyridin- 4-yl)ethoxy)-1H- indazol-3-yl)-3- fluoropyridin-2-yl)- 3-((1-methyl-1H- pyrazol-3-yl) methyl)pyrrolidin- 3-amine | LCMS m/z = 581.4 (M + H); 1HNMR (400 MHz, DMSO-d6) δ 1H NMR (400 MHz, DMSO- d6) δ 13.07 (s, 1H), 8.58 (d, J = 5.6 Hz, 2H), 8.36 (d, J = 6.4 Hz, 1H), 7.67 (dd, J = 14.8 Hz, 1.6 Hz, 1H), 7.62 (d, J = 2.0 Hz, 1H), 7.46 (d, J = 9.2 Hz, 1H), 7.20 (d, J = 2.0 Hz, 1H), 7.09 (dd, J = 9.2 Hz, 2.0 Hz, 1H), 6.16-6.11 (m, 2H), 3.81 (s, 3H), 3.77-3.52 (m, 4H), 2.88 (s, 2H), 2.06-1.98 (m, 1H), 1.87-1.82 (m, 1H), 1.76 (d, J = 6.5 Hz, 3H) |
| Example 429. | 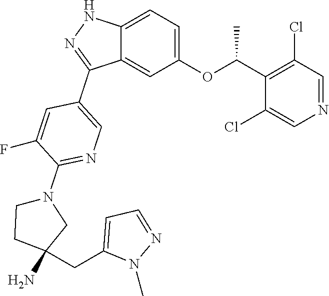 [SMILES: C[C@@H](Oc1ccc2[nH]nc(-c3cnc(N4CC[C@@](N)(Cc5ccnn5C)C4)c(F)c3)c2c1)c1c(Cl)cncc1Cl] | (R)-1-(5-(5-((R)-1- (3,5-dichloropyridin- 4-yl)ethoxy)-1H- indazol-3-yl)-3- fluoropyridin-2-yl)- 3-((1-methyl-1H- pyrazol-3-yl) methyl)pyrrolidin- 3-amine | LCMS m/z = 581.3 (M + H); 1HNMR (400 MHz, DMSO-d6) δ 1H NMR (400 MHz, DMSO- d6) δ 13.08 (s, 1H), 8.58 (d, J = 4.8 Hz, 2H), 8.37 (d, J = 6.8 Hz, 1H), 7.68 (dd, J = 14.4 Hz, 1.6 Hz, 1H), 7.63 (d, J = 2.0 Hz, 1H), 7.47 (d, J = 8.8 Hz, 1H), 7.20 (d, J = 2.0 Hz, 1H), 7.10 (dd, J =8.8 Hz, 2.0 Hz, 1H), 6.16-6.12 (m, 2H), 3.81 (s, 3H), 3.75-3.56 (m, 4H), 2.91 (s, 2H), 2.09-2.01 (m, 1H), 1.92-1.86 (m, 1H), 1.76 (d, J = 6.8 Hz, 3H) |
| Example 430. | 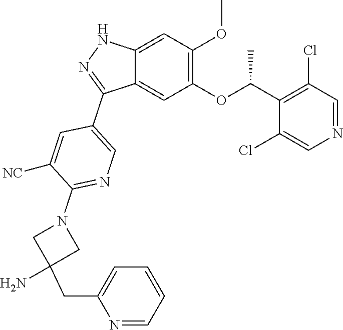 [SMILES: COc1cc2[nH]nc(-c3cnc(N4CC(N)(Cc5ccccn5)C4)c(C#N)c3)c2cc1O[C@H](C)c1c(Cl)cncc1Cl] | (R)-2-(3-amino-3- (pyridin-2- ylmethyl)azetidin-1- yl)-5-(5-(1-(3,5- dichloropyridin-4- yl)ethoxy)-6- methoxy-1H- indazol-3- yl)nicotinonitrile | LCMS m/z = 601.3 (M + H); 1HNMR (400 MHz, DMSO-d6) δ 12.96 (s, 1H), 8.69 (d, J = 2.4 Hz, 1H), 8.59 (s, 2H), 8.54-8.50 (m, 1H), 8.04 (d, J = 2.4 Hz, 1H), 7.74 (td, J = 7.6 Hz, 2.0 Hz, 1H), 7.40 (d, J = 7.6 Hz, 1H), 7.27-7.22 (m, 1H), 7.02- 7.00 (m, 2H), 5.99 (q, J = 6.4 Hz, 1H), 4.41 (d, J = 8.8 Hz, 2H), 4.02 (d, J = 8.8 Hz, 2H), 3.86 (s, 3H), 3.18 (s, 2H), 2.86 (brs, 2H), 1.76 (d, J = 6.4 Hz, 3H) |
| Example 431. | 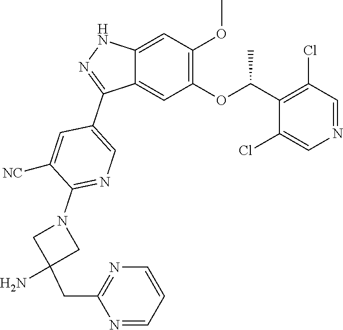 [SMILES: COc1cc2[nH]nc(-c3cnc(N4CC(N)(Cc5ncccn5)C4)c(C#N)c3)c2cc1O[C@H](C)c1c(Cl)cncc1Cl] | (R)-2-(3-amino-3- (pyrimidin-2- ylmethyl)azetidin-1- yl)-5-(5-(1-(3,5- dichloropyridin-4- yl)ethoxy)-6- methoxy-1H- indazol-3- yl)nicotinonitrile | LCMS m/z = 602.3 (M + H); 1HNMR (400 MHz, DMSO-d6) δ 12.97 (s, 1H), 8.78 (d, J = 4.8 Hz, 2H), 8.69 (d, J = 2.4 Hz, 1H), 8.59 (s, 2H), 8.04 (d, J = 2.8 Hz, 1H), 7.38 (t, J = 5.2 Hz, 1H), 7.03-7.01 (m, 2H), 6.00 (q, J = 6.8 Hz, 1H), 4.46 (d, J = 8.8 Hz, 2H), 4.08 (d, J = 9.2 Hz, 2H), 3.86 (s, 3H), 2.67 (brs, 2H), 1.76 (d, J = 6.8 Hz, 3H) |
| Example 432. | 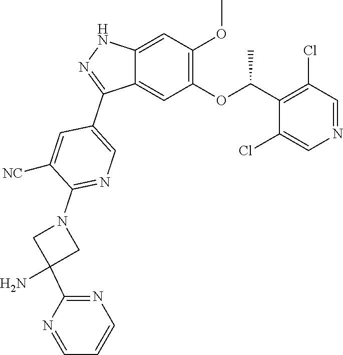 [SMILES: COc1cc2[nH]nc(-c3cnc(N4CC(N)(c5ncccn5)C4)c(C#N)c3)c2cc1O[C@H](C)c1c(Cl)cncc1Cl] | (R)-2-(3-amino-3- (pyrimidin-2- yl)azetidin-1-yl)-5- (5-(1-(3,5- dichloropyridin-4- yl)ethoxy)-6- methoxy-1H- indazol-3- yl)nicotinonitrile | LCMS m/z = 588.4 (M + H); 1HNMR (400 MHz, DMSO-d6) δ 12.98 (s, 1H), 8.90 (d, J = 4.8 Hz, 2H), 8.75 (d, J = 2.4 Hz, 1H), 8.59 (s, 2H), 8.10 (d, J = 2.0 Hz, 1H), 7.48 (t, J = 5.2 Hz, 1H), 7.05 (s, 1H), 7.00 (s, 1H), 6.00 (q, J = 6.4 Hz, 1H), 4.77 (d, J = 8.8 Hz, 2H), 4.37 (d, J = 8.8 Hz, 2H), 3.86 (s, 3H), 1.76 (d, J = 6.8 Hz, 3H) |
| Example 433. | 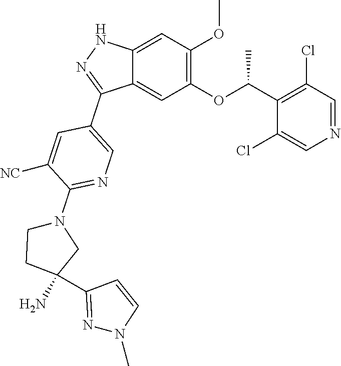 [SMILES: COc1cc2[nH]nc(-c3cnc(N4CC[C@](N)(c5ccn(C)n5)C4)c(C#N)c3)c2cc1O[C@H](C)c1c(Cl)cncc1Cl] | 2-((R)-3-amino-3- (1-methyl-1H- pyrazol-3- yl)pyrrolidin-1-yl)- 5-(5-((R)-1-(3,5- dichloropyridin-4- yl)ethoxy)-6- methoxy-1H- indazol-3- yl)nicotinonitrile | LCMS m/z = 604.3 (M + H); 1HNMR (400 MHz, DMSO-d6) δ 12.95 (s, 1H), 8.72 (d, J = 2.4 Hz, 1H), 8.59 (s, 2H), 8.06 (d, J = 2.0 Hz, 1H), 7.63 (d, J = 2.0 Hz, 1H), 7.04 (s, 1H), 7.01 (s, 1H), 6.35 (d, J = 2.0 Hz, 1H), 6.01 (q, J = 6.4 Hz, 1H), 4.06- 4.00 (m, 2H), 3.91-3.85 (m, 5H), 3.82 (s, 3H), 2.40-2.34 (m, 1H), 2.11-2.08 (m, 1H), 1.76 (d, J = 6.8 Hz, 3H) |
| Example 434. | 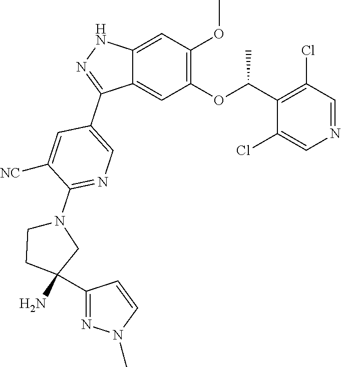 [SMILES: COc1cc2[nH]nc(-c3cnc(N4CC[C@@](N)(c5ccn(C)n5)C4)c(C#N)c3)c2cc1O[C@H](C)c1c(Cl)cncc1Cl] | 2-((S)-3-amino-3-(1- methyl-1H-pyrazol- 3-yl)pyrrolidin-1- yl)-5-(5-((R)-1-(3,5- dichloropyridin-4- yl)ethoxy)-6- methoxy-1H- indazol-3- yl)nicotinonitrile | LCMS m/z = 604.3 (M + H); 1HNMR (400 MHz, DMSO-d6) δ 12.97 (s, 1H), 8.72 (d, J = 2.4 Hz, 1H), 8.59 (s, 2H), 8.04 (d, J = 2.4 Hz, 1H), 7.63 (d, J = 2.0 Hz, 1H), 7.03 (s, 1H), 7.00 (s, 1H), 6.35 (d, J = 2.0 Hz, 1H), 6.00 (q, J = 6.8 Hz, 1H), 4.06- 4.03 (m, 2H), 3.90-3.85 (m, 5H), 3.82 (s, 3H), 2.40-2.33 (m, 1H), 2.11-2.07 (m, 1H), 1.76 (d, J = 6.8 Hz, 3H) |
| Example 435. | 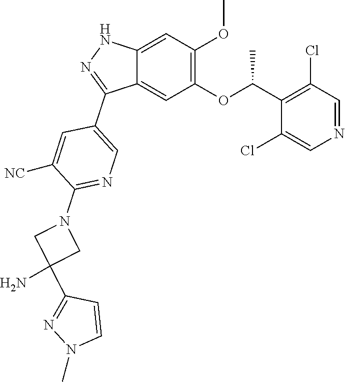 [SMILES: COc1cc2[nH]nc(-c3cnc(N4CC(N)(c5ccn(C)n5)C4)c(C#N)c3)c2cc1O[C@H](C)c1c(Cl)cncc1Cl] | (R)-2-(3-amino-3- (1-methyl-1H- pyrazol-3- yl)azetidin-1-yl)-5- (5-(1-(3,5- dichloropyridin-4- yl)ethoxy)-6- methoxy-1H- indazol-3- yl)nicotinonitrile | LCMS m/z = 590.3 (M + H); 1HNMR (400 MHz, DMSO-d6) δ 12.97 (s, 1H), 8.74 (d, J = 2.0 Hz, 1H), 8.59 (s, 2H), 8.09 (d, J = 2.0 Hz, 1H), 7.66 (d, J = 2.0 Hz, 1H), 7.05 (s, 1H), 7.01 (s, 1H), 6.37 (d, J = 1.6 Hz, 1H), 6.00 (q, J = 6.8 Hz, 1H), 4.58 (d, J = 8.8 Hz, 2H), 4.31 (d, J = 8.4 Hz, 2H), 3.86 (s, 3H), 3.83 (s, 3H), 1.76 (d, J = 6.8 Hz, 3H) |
| Example 436. | 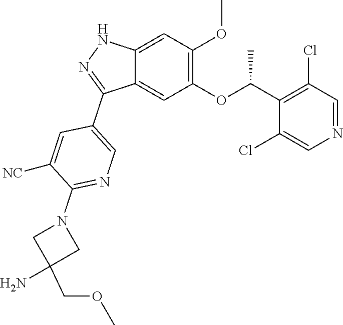 [SMILES: COCC1(N)CN(c2ncc(-c3n[nH]c4cc(OC)c(O[C@H](C)c5c(Cl)cncc5Cl)cc34)cc2C#N)C1] | 2-[3-amino-3- (methoxy- methyl)aze- tidin-1-yl]-5-[5- [(1R)-1-(3,5- dichloro-4- pyridyl)ethoxy]-6- methoxy-1H- indazol-3- yl]pyridine-3- carbonitrile | LCMS m/z = 554.4 (M + H); 1HNMR (400 MHz, DMSO-d6) δ 8.93 (br s, 2H), 8.76 (d, J = 2.2 Hz, 1H), 8.58 (s, 2H), 8.16 (d, J = 2.3 Hz, 1H), 7.05 (s, 1H), 7.02 (s, 1H), 5.99 (q, J = 6.7 Hz, 1H), 4.45-4.33 (m, 4H), 3.85 (s, 3H), 3.81 (s, 2H), 3.42 (s, 3H), 1.76 (d, J = 6.7 Hz, 3H) |
| Example 437. | 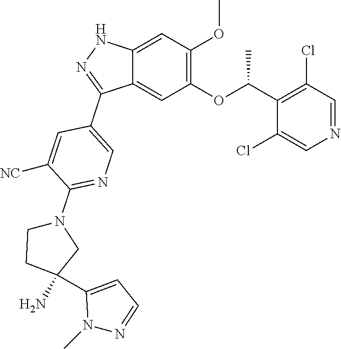 [SMILES: COc1cc2[nH]nc(-c3cnc(N4CC[C@](N)(c5ccnn5C)C4)c(C#N)c3)c2cc1O[C@H](C)c1c(Cl)cncc1Cl] | 2-((R)-3-amino-3- (1-methyl-1H- pyrazol-5- yl)pyrrolidin-1-yl)- 5-(5-((R)-1-(3,5- dichloropyridin-4- yl)ethoxy)-6- methoxy-1H- indazol-3- yl)nicotinonitrile | LCMS m/z = 604.3 (M + H); 1HNMR (400 MHz, DMSO-d6) δ 12.96 (s, 1H), 8.75 (d, J = 2.0 Hz, 1H), 8.59 (s, 2H), 8.08 (d, J = 2.4 Hz, 1H), 7.31 (d, J = 2.0 Hz, 1H), 7.05 (s, 1H), 7.01 (s, 1H), 6.16 (d, J = 1.6 Hz, 1H), 6.01 (q, J = 6.8 Hz, 1H), 4.13- 4.00 (m, 6H), 3.90-3.86 (m, 4H), 2.43-2.32 (m, 2H), 1.78 (d, J = 6.8 Hz, 3H) |
| Example 438. | 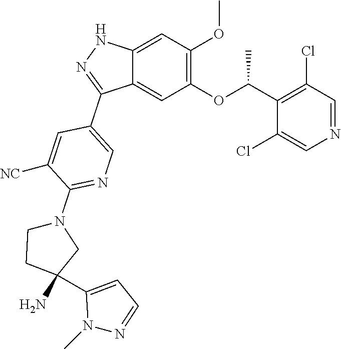 [SMILES: COc1cc2[nH]nc(-c3cnc(N4CC[C@@](N)(c5ccnn5C)C4)c(C#N)c3)c2cc1O[C@H](C)c1c(Cl)cncc1Cl] | 2-((S)-3-amino-3-(1- methyl-1H-pyrazol- 5-yl)pyrrolidin-1- yl)-5-(5-((R)-1-(3,5- dichloropyridin-4- yl)ethoxy)-6- methoxy-1H- indazol-3- yl)nicotinonitrile | LCMS m/z = 604.3 (M + H); 1HNMR (400 MHz, DMSO-d6) δ 12.96 (s, 1H), 8.76 (d, J = 2.8 Hz, 1H), 8.60 (s, 2H), 8.08 (d, J = 2.4 Hz, 1H), 7.31 (d, J = 1.6 Hz, 1H), 7.04 (s, 1H), 7.01 (s, 1H), 6.17 (d, J = 1.6 Hz, 1H), 6.01 (q, J = 6.8 Hz, 1H), 4.13- 4.01 (m, 6H), 3.90-3.84 (m, 4H), 2.45-2.31 (m, 2H), 1.77 (d, J = 6.8 Hz, 3H) |
| Example 439. | 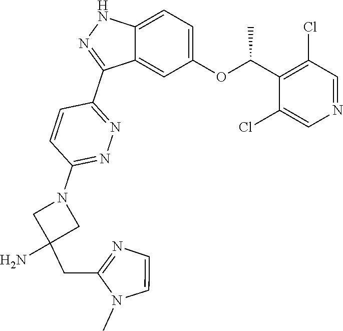 [SMILES: C[C@@H](Oc1ccc2[nH]nc(-c3ccc(N4CC(N)(Cc5nccn5C)C4)nn3)c2c1)c1c(Cl)cncc1Cl] | (R)-1-(6-(5-(1-(3,5- dichloropyridin-4- yl)ethoxy)-1H- indazol-3- yl)pyridazin-3-yl)-3- ((1-methyl-1H- imidazol-2- yl)methyl)azetidin- 3-amine | LCMS m/z = 550.3 (M + H); 1HNMR (400 MHz, DMSO-d6) δ 13.16 (s, 1H), 8.56 (s, 2H), 7.97-7.94 (m, 2H), 7.47 (d, J = 9.2 Hz, 1H), 7.11-7.07 (m, 2H), 6.90 (d, J = 9.2 Hz, 1H), 6.82 (s, 1H), 6.08 (q, J = 6.6 Hz, 1H), 4.16 (d, J = 8.2 Hz, 2H), 3.89 (d, J = 8.2 Hz, 2H), 3.62 (s, 3H), 3.11 (s, 2H), 2.77 (brs, 2H), 1.77 (d, J = 6.8 Hz, 3H) |
| Example 440. | 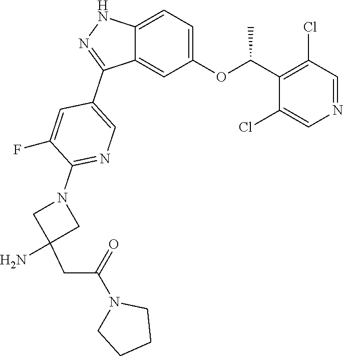 [SMILES: C[C@@H](Oc1ccc2[nH]nc(-c3cnc(N4CC(N)(CC(=O)N5CCCC5)C4)c(F)c3)c2c1)c1c(Cl)cncc1Cl] | (R)-2-(3-amino-1- (5-(5-(1-(3,5- dichloropyridin-4- yl)ethoxy)-1H- indazol-3-yl)-3- fluoropyridin-2-yl) azetidin-3-yl)-1- (pyrrolidin-1-yl) ethanone | LCMS m/z = 584.3 (M + H); 1HNMR (400 MHz, DMSO-d6) δ 13.14 (s, 1H), 8.57 (s, 2H), 8.34 (d, J = 1.6 Hz, 1H), 7.69 (dd, J = 13.2 Hz, 1.6 Hz, 1H), 7.47 (d, J = 9.2 Hz, 1H), 7.19 (d, J = 2.0 Hz, 1H), 7.10 (dd, J = 9.2 Hz, 2.4 Hz, 1H), 6.14 (q, J = 6.8 Hz, 1H), 4.08 (dd, J = 8.4 Hz, 1.6 Hz, 2H), 3.95 (d, J = 8.4 Hz, 1H), 3.44 (t, J = 6.8 Hz, 4H), 2.83 (s, 2H), 1.92-1.85 (m, 2H), 1.82-1.78 (m, 2H), 1.76 (d, J = 6.4 Hz, 3H) |
| Example 441. | 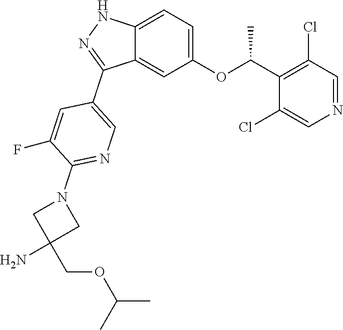 [SMILES: CC(C)OCC1(N)CN(c2ncc(-c3n[nH]c4ccc(O[C@H](C)c5c(Cl)cncc5Cl)cc34)cc2F)C1] | 1-[5-[5-[(1R)-1-(3,5- dichloro-4- pyridyl)ethoxy]-1H- indazol-3-yl]-3- fluoro-2-pyridyl]-3- (isopropoxy- methyl)a- zetidin-3-amine | LCMS m/z = 545.2 (M + H); 1HNMR (400 MHz, DMSO-d6) δ 13.09 (br s, 1H), 8.57 (s, 2H), 8.36 (t, J = 1.6 Hz, 1H), 7.68 (dd, J = 1.8, 13.2 Hz, 1H), 7.47 (d, J = 8.9 Hz, 1H), 7.20 (d, J = 1.8 Hz, 1H), 7.10 (dd, J = 2.2, 9.0 Hz, 1H), 6.14 (q, J = 6.6 Hz, 1H), 4.02 (dd, J = 1.8, 8.2 Hz, 2H), 3.84 (br d, J = 7.5 Hz, 2H), 3.67-3.60 (m, 1H), 3.51-3.49 (m, 2H), 1.76 (d, J = 6.6 Hz, 3H), 1.13 (d, J = 6.1 Hz, 6H) |
| Example 442. | 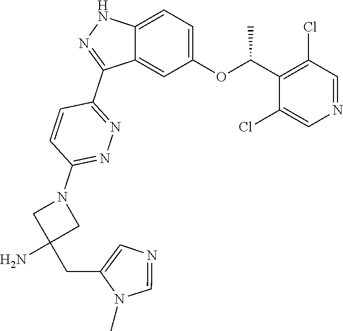 [SMILES: C[C@@H](Oc1ccc2[nH]nc(-c3ccc(N4CC(N)(Cc5cncn5C)C4)nn3)c2c1)c1c(Cl)cncc1Cl] | (R)-1-(6-(5-(1-(3,5- dichloropyridin-4- yl)ethoxy)-1H- indazol-3- yl)pyridazin-3-yl)-3- ((1-methyl-1H- imidazol-5- yl)methyl)azetidin- 3-amine | LCMS m/z = 550.3 (M + H); 1HNMR (400 MHz, DMSO-d6) δ 13.19 (s, 1H), 8.56 (s, 2H), 7.98 (d, J = 9.2 Hz, 1H), 7.95 (d, J = 2.0 Hz, 1H), 7.56 (s, 1H), 7.48 (d, J = 8.8 Hz, 1H), 7.11 (dd, J = 8.8 Hz, 2.4 Hz, 1H), 6.93 (d, J = 9.6 Hz, 1H), 6.86 (s, 1H), 6.10 (q, J = 6.8 Hz, 1H), 4.10 (d, J = 8.0 Hz, 2H), 3.86 (d, J = 8.4 Hz, 2H), 3.62 (s, 3H), 3.03 (s, 2H), 1.78 (d, J = 6.4 Hz, 3H). |
| Example 443. | 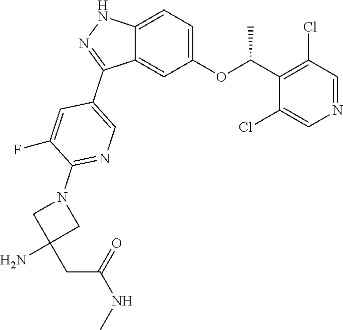 [SMILES: CNC(=O)CC1(N)CN(c2ncc(-c3n[nH]c4ccc(O[C@H](C)c5c(Cl)cncc5Cl)cc34)cc2F)C1] | (R)-2-(3-amino-1- (5-(5-(1-(3,5- dichloropyridin-4- yl)ethoxy)-1H- indazol-3-yl)-3- fluoropyridin-2- yl)azetidin-3-yl)-N- methylacetamide | LCMS m/z = 544.4 (M + H); 1HNMR (400 MHz, DMSO-d6) δ 13.11 (s, 1H), 8.59 (s, 2H), 8.36 (s, 1H), 8.03 (d, J = 4.8 Hz, 1H), 7.69 (d, J = 13.2 Hz, 1H), 7.47 (d, J = 8.4 Hz, 1H), 7.19 (s, 1H), 7.10 (d, J = 9.2 Hz, 1H), 6.14 (q, J = 6.4 Hz, 1H), 4.06 (d, J = 8.0 Hz, 2H), 3.88 (d, J = 7.6 Hz, 2H), 2.61- 2.58 (m, 5H), 1.76 (d, J = 7.6 Hz, 3H) |
| Example 444. | 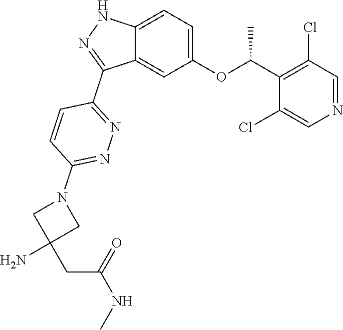 [SMILES: CNC(=O)CC1(N)CN(c2ccc(-c3n[nH]c4ccc(O[C@H](C)c5c(Cl)cncc5Cl)cc34)nn2)C1] | (R)-2-(3-amino-1- (6-(5-(1-(3,5- dichloropyridin-4- yl)ethoxy)-1H- indazol-3- yl)pyridazin-3- yl)azetidin-3-yl)-N- methylacetamide | LCMS m/z = 527.2 (M+H); 1HNMR (400 MHz, DMSO-d6) δ 13.18 (s, 1H), 8.59 (s, 2H), 8.04 (q, J = 4.8 Hz, 1H), 7.97- 7.94 (m, 2H), 7.47 (d, J = 8.8 Hz, 1H), 7.09 (dd, J = 9.2 Hz, 2.8 Hz, 1H), 6.89 (d, J = 9.6 Hz, 1H), 6.08 (q, J = 6.4 Hz, 1H), 4.07 (d, J = 8.4 Hz, 2H), 3.85 (d, J = 8.4 Hz, 2H), 2.87 (brs, 2H), 2.62-2.60 (m, 5H), 1.77 (d, J = 6.8 Hz, 3H) |
| Example 445. | 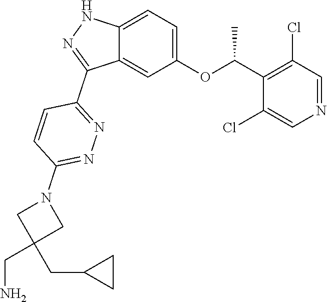 [SMILES: C[C@@H](Oc1ccc2[nH]nc(-c3ccc(N4CC(CN)(CC5CC5)C4)nn3)c2c1)c1c(Cl)cncc1Cl] | (R)-(3-(cyclo- propylmethyl)- 1-(6-(5-(1-(3,5- dichloropyridin-4- yl)ethoxy)-1H- indazol-3- yl)pyridazin-3- yl)azetidin-3- yl)methanamine | LCMS m/z = 524.2 (M + H); 1HNMR (400 MHz, DMSO-d6) δ 13.18 (brs, 1H), 8.57 (s, 2H), 7.97-7.94 (m, 2H), 7.46 (d, J = 8.8 Hz, 1H), 7.09 (dd, J = 8.8 Hz, 2.4 Hz, 1H), 6.85 (d, J = 9.2 Hz, 1H), 6.08 (q, J =6.4 Hz, 1H), 3.92 (d, J = 8.4 Hz, 2H), 3.85 (d, J = 8.0 Hz, 2H), 2.89 (s, 2H), 1.77 (d, J = 6.4 Hz, 3H), 1.61(d, J = 6.8 Hz, 2H), 0.75-0.71 (m, 1H), 0.47-0.43 (m, 2H), 0.16-0.12 (m, 2H) |
| Example 446. | 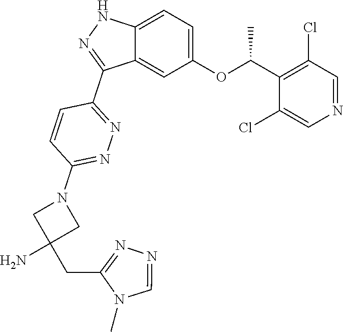 [SMILES: C[C@@H](Oc1ccc2[nH]nc(-c3ccc(N4CC(N)(Cc5nncn5C)C4)nn3)c2c1)c1c(Cl)cncc1Cl] | (R)-1-(6-(5-(1-(3,5- dichloropyridin-4- yl)ethoxy)-1H- indazol-3- yl)pyridazin-3-yl)-3- ((4-methyl-4H- 1,2,4-triazol-3- yl)methyl)azetidin- 3-amine | LCMS m/z = 551.5 (M + H); 1HNMR (400 MHz, DMSO-d6) δ 13.17 (s, 1H), 8.60 (s, 2H), 8.40 (s, 1H), 7.98-7.95 (m, 2H), 7.47 (d, J = 9.2 Hz, 1H), 7.09 (dd, J = 8.8 Hz, 2.4 Hz, 1H), 6.92 (d, J = 9.6 Hz, 1H), 6.08 (q, J = 6.4 Hz, 1H), 4.20 (d, J = 8.0 Hz, 2H), 3.89 (d, J = 8.4 Hz, 2H), 3.65 (s, 3H) ), 3.20 (s, 2H), 1.77 (d, J = 6.4 Hz, 3H) |
| Example 447. | 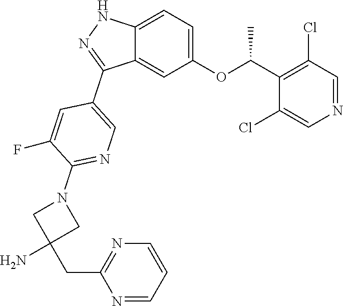 [SMILES: C[C@@H](Oc1ccc2[nH]nc(-c3cnc(N4CC(N)(Cc5ncccn5)C4)c(F)c3)c2c1)c1c(Cl)cncc1Cl] | (R)-1-(5-(5-(1-(3,5- dichloropyridin-4- yl)ethoxy)-1H- indazol-3-yl)-3- fluoropyridin-2-yl)- 3-(pyrimidin-2- ylmethyl)azetidin-3- amine | LCMS m/z = 564.4 (M + H); 1HNMR (400 MHz, DMSO-d6) δ 13.14 (s, 1H), 8.80 (d, J = 4.8 Hz, 2H), 8.57 (s, 2H), 8.37 (t, J = 1.6 Hz, 1H), 7.72 (dd, J = 13.2 Hz, 2.0 Hz, 1H), 7.48 (d, J = 9.2 Hz, 1H), 7.43 (t, J = 4.8 Hz, 1H), 7.19 (d, J = 2.0 Hz, 1H), 7.11 (dd, J = 9.2 Hz, 2.4 Hz, 1H), 6.32-6.05 (m, 3H), 4.34 (dd, J = 9.2 Hz, 1.6 Hz, 2H), 4.14 (d, J = 8.8 Hz, 2H), 3.51 (s, 2H), 1.76 (d, J = 6.4 Hz, 3H) |
| Example 448. | 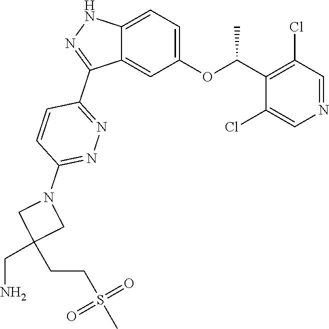 [SMILES: C[C@@H](Oc1ccc2[nH]nc(-c3ccc(N4CC(CN)(CCS(C)(=O)=O)C4)nn3)c2c1)c1c(Cl)cncc1Cl] | (R)-(1-(6-(5-(1-(3,5- dichloropyridin-4- yl)ethoxy)-1H- indazol-3- yl)pyridazin-3-yl)-3- (2-(methyl- sulfonyl)ethyl) azetidin-3- yl)methanamine | LCMS m/z = 576.4 (M + H); 1HNMR (400 MHz, DMSO-d6) δ 13.22 (s, 1H), 8.55 (s, 2H), 8.02 (d, J = 9.6 Hz, 1H), 7.93 (d, J = 2.0 Hz, 1H), 7.49 (d, J = 9.2 Hz, 1H), 7.11 (dd, J = 8.8 Hz, 2.4 Hz, 1H), 6.87 (d, J = 9.2 Hz, 1H), 6.10 (q, J = 6.4 Hz, 1H), 4.03 (d, J = 8.4 Hz, 2H), 3.94 (d, J = 8.0 Hz, 2H), 3.38- 3.35 (m, 2H), 3.27-3.19 (m, 2H), 3.04 (s, 3H), 2.21-2.17 (m, 2H), 1.77 (d, J = 6.8 Hz, 3H) |
| Example 449. | 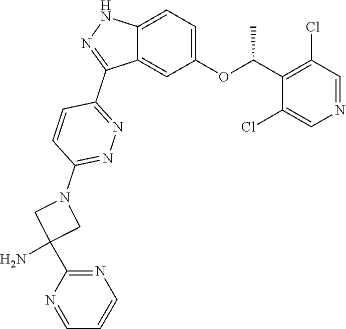 [SMILES: C[C@@H](Oc1ccc2[nH]nc(-c3ccc(N4CC(N)(c5ncccn5)C4)nn3)c2c1)c1c(Cl)cncc1Cl] | (R)-1-(6-(5-(1-(3,5- dichloropyridin-4- yl)ethoxy)-1H- indazol-3- yl)pyridazin-3-yl)-3- (pyrimidin-2- yl)azetidin-3-amine | LCMS m/z = 534.4 (M + H); 1HNMR (400 MHz, DMSO-d6) δ 13.20 (s, 1H), 8.92 (d, J = 4.8 Hz, 2H), 8.56 (s, 2H), 8.01 (d, J = 9.2 Hz, 1H), 7.96 (d, J = 2.4 Hz, 1H), 7.51 (d, J = 4.4 Hz, 1H), 7.48 (d, J = 8.8 Hz, 1H), 7.10 (dd, J = 8.8 Hz, 2.4 Hz, 1H), 7.00 (d, J = 9.2 Hz, 1H), 6.08 (q, J = 6.8 Hz, 1H), 4.63 (d, J = 8.4 Hz, 2H), 4.46 (brs, 2H), 4.25 (d, J = 8.4 Hz, 2H), 1.77 (d, J = 6.8 Hz, 3H) |

### Kinase Assays

Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. Streptavidin-coated magnetic beads were treated with biotinylated small molecule ligands for 30 minutes at room temperature to generate affinity resins for kinase assays. The liganded beads were blocked with excess biotin and washed with blocking buffer SeaBlock (Pierce), 1% BSA, 0.05% Tween 20, 1 mM DTT to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT).

Test compounds were prepared as 111× stocks in 100% DMSO. Kds were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 ml. The assay plates were incubated at room temperature with shaking for 1 hour and the affinity beads were washed with wash buffer (lx PBS, 0.05% Tween 20). The beads were then re-suspended in elution buffer (lx PBS, 0.05% Tween 20, 0.5 M non-biotinylated affinity ligand) and incubated at room temperature with shaking for 30 minutes. The kinase concentration in the eluates was measured by qPCR.

### Binding Constants (Kds)

Binding constants were calculated with a standard dose-response curve using the Hill equation:

$$Response = Background + \frac{Signal - Background}{1 + ( \frac{K {d}^{Hill Slope}}{D o s {e}^{Hill Slope}} )}$$

The Hill Slope was set to −1. Curves were fitted using a non-linear least square fit with the Levenberg-Marquardt algorithm.

TABLE 4
A = 1-50 nM
B = >50 to 300 nM
C = >300 to 1000 nM
D => 1000 nM

| | | | | | FGFR3 |
|---|---|---|---|---|---|
| | FGFR1 | FGFR2 | FGFR3 | FGFR4 | (V555M) |
| Example | (K_{d} nM) | (K_{d} nM) | (K_{d} nM) | (K_{d} nM) | (K_{d} nM) |
| 1. | A | A | A | A | A |
| 2. | B | B | A | A | A |
| 3. | B | C | A | A | A |
| 4. | B | B | A | A | A |
| 5. | D | D | B | A | B |
| 6. | B | A | A | A | A |
| 7. | B | B | A | A | A |
| 8. | A | A | A | A | A |
| 9. | A | A | A | A | A |
| 10. | A | A | A | A | A |
| 11. | B | A | A | A | A |
| 12. | A | A | A | A | A |
| 13. | D | D | B | A | B |
| 14. | A | A | A | A | A |
| 15. | A | A | A | A | A |
| 16. | A | A | A | A | A |
| 17. | A | A | A | A | A |
| 18. | B | B | A | A | A |
| 19. | B | C | A | A | A |
| 20. | A | A | A | A | A |
| 21. | B | B | A | A | A |
| 22. | B | B | A | A | A |
| 23. | A | A | A | A | A |
| 24. | A | A | A | A | A |
| 25. | A | A | A | A | A |
| 26. | A | A | A | A | A |
| 27. | A | B | A | A | A |
| 28. | A | A | A | A | A |
| 29. | B | B | A | A | A |
| 30. | A | A | A | A | A |
| 31. | A | B | A | A | A |
| 32. | A | B | A | A | A |
| 33. | B | A | A | A | A |
| 34. | B | B | A | A | A |
| 35. | A | A | A | A | A |
| 36. | B | B | A | A | A |
| 37. | A | A | A | A | A |
| 38. | C | B | A | A | A |
| 39. | B | B | A | A | A |
| 40. | B | B | A | A | A |
| 41. | A | A | A | A | A |
| 42. | A | A | A | A | A |
| 43. | A | A | A | A | A |
| 44. | A | A | A | A | A |
| 45. | B | B | A | A | A |
| 46. | D | D | C | A | B |
| 47. | A | A | A | A | A |
| 48. | A | A | A | A | A |
| 49. | A | A | A | A | A |
| 50. | A | A | A | A | A |
| 51. | A | A | A | A | A |
| 52. | A | A | A | A | A |
| 53. | A | A | A | A | A |
| 54. | A | A | A | A | A |
| 55. | A | A | A | A | A |
| 56. | A | A | A | A | A |
| 57. | A | A | A | A | A |
| 58. | B | A | A | A | A |
| 59. | A | A | A | A | A |
| 60. | B | C | A | A | A |
| 61. | A | A | A | A | A |
| 62. | A | A | A | A | A |
| 63. | A | A | A | A | A |
| 64. | A | A | A | A | A |
| 65. | B | A | A | A | A |
| 66. | A | A | A | A | A |
| 67. | A | A | A | A | A |
| 68. | A | A | A | A | A |
| 69. | A | A | A | A | A |
| 70. | A | A | A | A | A |
| 71. | A | A | A | A | A |
| 72. | A | A | A | A | A |
| 73. | A | A | A | A | A |
| 74. | A | A | A | A | A |
| 75. | C | B | A | B | A |
| 76. | A | A | A | A | A |
| 77. | A | A | A | A | A |
| 78. | A | A | A | A | A |
| 79. | A | A | A | A | A |
| 80. | A | A | A | A | A |
| 81. | A | A | A | A | A |
| 82. | A | A | A | A | A |
| 83. | A | A | A | A | A |
| 84. | A | A | A | A | A |
| 85. | A | A | A | A | A |
| 86. | A | A | A | A | A |
| 87. | A | B | A | A | A |
| 88. | A | B | A | A | A |
| 89. | A | A | A | A | A |
| 90. | A | A | A | A | A |
| 91. | A | A | A | A | A |
| 92. | A | A | A | A | A |
| 93 | A | A | A | A | A |
| 94 | A | A | A | A | A |
| 95. | A | A | A | A | A |
| 96. | A | A | A | A | A |
| 97. | A | A | A | A | A |
| 98. | B | B | A | A | A |
| 99. | A | A | A | A | A |
| 100. | A | A | A | A | A |
| 101. | A | A | A | A | A |
| 102. | A | A | A | A | A |
| 103. | A | A | A | A | A |
| 104. | A | A | A | A | A |
| 105. | A | A | A | A | A |
| 106. | A | A | A | A | A |
| 107. | A | A | A | A | A |
| 108. | A | A | A | A | A |
| 109. | A | A | A | A | A |
| 110. | A | A | A | A | A |
| 111. | A | A | A | A | A |
| 112. | A | A | A | A | A |
| 113. | A | A | A | A | A |
| 114. | A | A | A | A | A |
| 115. | A | A | A | A | A |
| 116. | A | A | A | A | A |
| 117. | A | A | A | A | A |
| 118. | A | A | A | A | A |
| 119. | A | A | A | A | A |
| 120. | A | B | A | A | A |
| 121. | A | A | A | A | A |
| 122. | B | B | A | A | A |
| 123. | B | A | A | A | A |
| 124. | B | B | A | A | A |
| 125. | A | A | A | A | A |
| 126. | A | A | A | A | A |
| 127. | A | A | A | A | A |
| 128. | A | A | A | A | A |
| 130. | A | A | A | A | A |
| 132. | A | A | A | A | A |
| 133. | A | B | A | A | A |
| 134. | B | B | A | A | A |
| 135. | B | A | A | A | A |
| 136. | A | A | A | A | A |
| 137. | B | B | A | A | A |
| 138. | A | A | A | A | A |
| 139. | A | A | A | A | A |
| 140. | A | A | A | A | A |
| 141. | A | A | A | A | A |
| 142. | A | A | A | A | A |
| 143. | A | A | A | A | A |
| 144. | A | A | A | A | A |
| 145. | A | A | A | A | A |
| 146. | A | A | A | A | A |
| 147. | A | A | A | A | A |
| 148. | A | A | A | A | A |
| 149. | A | A | A | A | A |
| 150. | A | | A | | |
| 151. | A | | A | | |
| 152. | A | A | A | A | |
| 153. | A | A | A | A | |
| 154. | A | A | A | A | A |
| 155. | A | A | A | A | A |
| 156. | A | A | A | A | A |
| 157. | A | A | A | A | A |
| 158. | A | A | A | A | A |
| 159. | A | A | A | A | A |
| 160. | A | A | A | A | A |
| 161. | A | A | A | A | A |
| 162. | A | A | A | A | A |
| 163. | A | A | A | A | A |
| 164. | A | A | A | A | A |
| 165. | A | A | A | A | A |
| 166. | A | A | A | A | A |
| 167. | A | A | A | A | A |
| 168. | B | B | A | B | A |
| 169. | C | D | B | B | A |
| 170. | D | D | B | C | B |
| 171. | A | A | A | A | A |
| 172. | C | B | B | B | A |
| 173. | C | C | B | C | A |
| 174. | C | D | B | B | B |
| 175. | D | D | B | B | B |
| 176. | C | D | B | B | B |
| 177. | B | C | A | B | A |
| 178. | A | A | A | A | A |
| 179. | A | A | A | A | A |
| 180. | A | A | A | A | A |
| 181. | A | A | A | A | A |
| 182. | A | A | A | A | A |
| 183. | C | D | B | B | B |
| 184. | C | C | A | B | A |
| 185. | C | C | B | B | B |
| 186. | A | A | A | A | A |
| 187. | D | C | B | C | B |
| 188. | A | A | A | A | A |
| 189. | A | A | A | A | A |
| 190. | C | C | A | C | A |
| 191. | C | C | B | C | A |
| 192. | C | C | B | C | A |
| 193. | A | A | A | A | A |
| 194. | A | A | A | A | A |
| 195. | A | A | A | A | A |
| 196. | D | D | C | C | B |
| 197. | C | D | B | B | A |
| 198. | A | A | A | A | A |
| 199. | A | A | A | A | A |
| 200. | C | D | B | B | A |
| 201. | C | C | B | C | C |
| 202. | C | D | B | B | A |
| 203. | A | A | A | A | A |
| 204. | A | A | A | A | A |
| 205. | B | C | B | B | A |
| 206. | A | A | A | A | A |
| 207. | A | A | A | A | A |
| 208. | D | D | B | C | B |
| 209. | A | A | A | A | A |
| 210. | B | A | A | A | A |
| 211. | A | A | A | A | A |
| 212. | A | A | A | A | A |
| 213. | A | A | A | A | A |
| 214. | A | A | A | A | A |
| 215. | A | A | A | A | A |
| 216. | A | A | A | A | A |
| 217. | A | A | A | A | A |
| 218. | A | A | A | A | A |
| 219. | A | A | A | A | A |
| 220. | A | A | A | A | A |
| 221. | A | A | A | A | A |
| 222. | A | A | A | A | A |
| 223. | A | A | A | A | A |
| 224. | A | A | A | A | A |
| 225. | A | A | A | A | A |
| 226. | A | A | A | A | A |
| 227. | A | A | A | A | A |
| 228. | A | A | A | A | A |
| 229. | A | A | A | A | A |
| 230. | A | A | A | A | A |
| 231. | A | A | A | A | A |
| 232. | A | A | A | A | A |
| 233. | A | A | A | A | A |
| 234. | A | A | A | A | A |
| 235. | A | A | A | A | A |
| 236. | A | A | A | A | A |
| 237. | A | A | A | A | A |
| 238. | A | | A | | |
| 239. | A | A | A | A | A |
| 240. | A | | A | | |
| 241. | A | | A | | |
| 242. | A | | A | | |
| 243. | A | | A | | |
| 244. | A | | A | | |
| 245. | B | | A | | |
| 246. | A | | A | | |
| 247. | A | | A | | |
| 248. | A | | A | | |
| 249. | A | | A | | |
| 250. | B | | A | | |
| 251. | A | | A | | |
| 252. | A | | A | | |
| 253. | B | | A | | |
| 254. | B | | A | | |
| 255. | A | | A | | |
| 256. | A | | A | | |
| 257. | B | | A | | |
| 258. | A | | A | | |
| 259. | A | | A | | |
| 260. | A | | A | | |
| 261. | A | | A | | |
| 262. | A | | A | | |
| 263. | A | | A | | |
| 264. | A | | A | | |
| 265. | A | | A | | |
| 266. | A | | A | | |
| 267. | A | | A | | |
| 268. | A | | A | | |
| 269. | A | | A | | |
| 270. | A | | A | | |
| 271. | A | | A | | |
| 272. | A | | A | | |
| 273. | A | | A | | |
| 274. | A | | A | | |
| 275. | A | | A | | |
| 276. | A | | A | | |
| 277. | A | | A | | |
| 278. | A | | A | | |
| 279. | A | | A | | |
| 280. | A | | A | | |
| 281. | A | | A | | |
| 282. | A | | A | | |
| 283. | A | | A | | |
| 284. | A | | A | | |
| 285. | A | | A | | |
| 286. | A | | A | | |
| 287. | A | | A | | |
| 288. | A | | A | | |
| 289. | A | | A | | |
| 290. | A | | A | | |
| 291. | A | | A | | |
| 292. | A | | A | | |
| 293. | A | | A | | |
| 294. | A | | A | | |
| 295. | A | | A | | |
| 296. | A | | A | | |
| 297. | A | | A | | |
| 298. | A | | A | | |
| 299. | A | | A | | |
| 300. | A | | A | | |
| 301. | A | | A | | |
| 302. | A | | A | | |
| 303. | A | | A | | |
| 304. | A | | A | | |
| 305. | A | | A | | |
| 306. | A | | A | | |
| 307. | A | | A | | |
| 308. | A | | A | | |
| 309. | A | | A | | |
| 310. | A | | A | | |
| 311. | A | | A | | |
| 312. | A | | A | | |
| 313. | A | | A | | |
| 314. | A | | A | | |
| 315. | A | | A | | |
| 316. | A | | A | | |
| 317. | B | B | A | A | A |
| 318. | D | D | D | B | D |
| 319. | D | D | D | B | C |
| 320. | B | B | A | A | A |
| 321. | A | A | A | A | A |
| 322. | D | D | D | B | C |
| 323. | D | D | D | A | C |
| 324. | D | D | D | A | C |
| 325. | A | A | A | A | A |
| 326. | D | D | D | A | D |
| 327. | D | D | D | B | D |
| 328. | A | A | A | A | A |
| 329. | A | A | A | A | A |
| 330. | C | C | B | A | B |
| 331. | C | C | B | A | B |
| 332. | D | D | C | A | C |
| 333. | C | D | B | A | B |
| 334. | A | A | A | A | A |
| 335. | D | D | C | A | C |
| 336. | A | A | A | A | A |
| 337. | A | A | A | A | A |
| 338. | A | A | A | A | A |
| 339. | A | A | A | A | A |
| 340. | A | A | A | A | A |
| 341. | A | A | A | A | A |
| 342. | A | A | A | A | |
| 343. | B | B | A | A | |
| 344. | A | A | A | A | |
| 345. | A | A | A | A | |
| 346. | A | A | A | A | |
| 347. | A | A | A | A | |
| 348. | A | A | A | A | |
| 349. | A | A | A | A | |
| 350. | B | A | A | A | |
| 351. | A | A | A | A | |
| 352. | A | A | A | A | |
| 353. | A | A | A | A | |
| 354. | A | A | A | A | |
| 355. | A | B | A | A | |
| 356. | A | A | A | A | |
| 357. | A | A | A | A | |
| 358. | A | B | A | A | |
| 359. | A | A | A | A | |
| 360. | A | A | A | A | |
| 361. | A | A | A | A | |
| 362. | A | A | A | A | |
| 363. | A | A | A | A | |
| 364. | A | A | A | A | |
| 365. | A | A | A | A | |
| 366. | A | A | A | A | |
| 367. | A | A | A | A | |
| 368. | A | A | A | A | |
| 369. | A | A | A | A | |
| 370. | A | A | A | A | |
| 371. | A | | A | | |
| 372. | A | | A | | |
| 373. | A | | A | | |
| 374. | A | | A | | |
| 375. | A | | A | | |
| 376. | A | | A | | |
| 377. | A | | A | | |
| 378. | A | | A | | |
| 379. | A | | A | | |
| 380. | A | | A | | |
| 381. | A | | A | | |
| 382. | A | | A | | |
| 383. | A | | A | | |
| 384. | A | | A | | |
| 385. | A | | A | | |
| 386. | A | | A | | |
| 387. | A | | A | | |
| 388. | A | | A | | |
| 389. | A | | A | | |
| 390. | A | | A | | |
| 391. | A | | A | | |
| 392. | A | | A | | |
| 393. | A | | A | | |
| 394. | A | | A | | |
| 395. | A | | A | | |
| 396. | A | | A | | |
| 397. | A | | A | | |
| 398. | A | | A | | |
| 399. | A | | A | | |
| 400. | A | | A | | |
| 401. | A | | A | | |
| 402. | A | | A | | |
| 403. | A | | A | | |
| 404. | A | | A | | |
| 405. | A | | A | | |
| 406. | A | | A | | |
| 407. | A | | A | | |
| 408. | A | | A | | |
| 409. | A | A | A | A | |
| 410. | A | A | A | A | |
| 411. | | | | | |
| 412. | A | | A | | |
| 413. | | | | | |
| 414. | | | | | |
| 415. | | | | | |
| 416. | A | | A | | |
| 417. | A | | A | | |
| 418. | | | | | |
| 419. | | | | | |
| 420. | | | | | |
| 421. | | | | | |
| 422. | | | | | |
| 423. | | | | | |
| 424. | A | | A | | |
| 425. | A | | A | | |
| 426. | A | | A | | |
| 427. | A | | A | | |
| 428. | A | | A | | |
| 429. | A | | A | | |

### Cell Viability Assays

Cell lines used for Cell Viability Assays

| Cell line | Mutation or Fusion | Source |
|---|---|---|
| KG-1 | FGFR 1-fusion | American Type Culture |
| | | Collection (Manassas, VA) |
| KATO-III | FGFR2 amplification | American Type Culture |
| | | Collection (Manassas, VA) |
| RT112/84 | FGFR3-fusion | Millipore Sigma (St. Louis, |
| | | MO) |
| HuH7 | FGF19 amplification | Sekisui Xenotech (Kansas |
| | | City, KS) |
| MDA-MB-453 | FGFR4-Y367C | American Type Culture |
| | | Collection (Manassas, VA) |

### Cancer Cell Line Cell Viability Assays

Experimental Purpose: To detect the change of intracellular ATP by Cell Titer-Glo® and to evaluate the inhibitory effect of the compounds on cancer cell lines by determining the in vitro IC₅₀ value of the compounds.

Cell Titer-Glo® 2.0 Luminescent cell viability assay reagent was purchased from Promega (Madison, WI). KG-1, KATO-III, and MDA-MB-453 cell lines were purchased from American Type Culture Collection (Manassas, VA). RT112/84 cell line was purchased from Millipore-Sigma (St. Louis, MO). HuH7 cells were purchased from Seikisui Xenotech (Kansas City, KS). RT112/84 and MDA-MB-453 cells were cultured in RPMI1640 media supplemented with 10% fetal bovine serum. KG-1 and KATO-III cell lines were cultured in IMDM media supplemented with 20% FBS. HuH7 cells were cultured in IMDM media supplemented with 10% FBS. Cultures were maintained at 37° C. in a humidified atmosphere of 5% CO₂ and 95% air.

Cell Viability Assay Procedure: Cells were plated in 96-well clear bottom/white plates (Corning #3903) at a range of densities depending on the optimal assay window (5,000-20,000 cells/well in 100p of media), incubated overnight. The next day, test compound DMSO stock solutions were made at 10 mM and 2 μM final concentration. Compounds were then added to cells in a 9-dose, 4-fold dilution series starting at 3 μM with an HP 300e Digital Dispenser (each dose was applied in triplicate). DMSO was backfilled to each well up to 301 nL total volume of test compound+DMSO, and a total of 301 nL DMSO was added to a control/no test compound well in triplicate. The cells in cell culture plates were incubated with the compounds at 37° C. and 5% CO₂ for 72 hours-120 hours depending on the cell line. Then 50 μl of Cell Titer Glo 2.0 reagent was added to each well of the cell culture plates. The contents were covered from light and mixed on an orbital shaker at room temperature for minimum of 10 min. Luminescence was recorded by a Clariostar Plus Microplate Reader (BMG Labtech, Cary, NC). Cells were assessed as a percentage of DMSO only treated control cells. Curves were plotted and IC₅₀ values were calculated using the GraphPad Prism 9 program based on a sigmoidal dose-response equation (log (inhibitor) vs. response—Variable slope, 4-parameter).

Cell Viability Assay Protocol Details

| | | | | | Complete | Cell | Passage |
|---|---|---|---|---|---|---|---|
| Cell Line | Phenotype | Vendor | Format | Duration | Media | #/well | # |
| KG1 | suspension | ATCC | 96-Well | 72 hr | IMDM | 20,000 | 5 to 25 |
| | | | 384-Well | | (+20% FBS) | 5,000 | |
| Kato III | adherent | ATCC | 96-Well | 72 hr | IMDM | 5,000 | 5 to 30 |
| | | | | | (+20% FBS) | | |
| RT112/84 | adherent | Millipore- | 96-Well | 72 hr | RPMI1640 | 10,000 | 15 to 45 |
| | | Sigma | 384-Well | | (+10% FBS) | 5,000 | |
| MDA-MB- | adherent | ATCC | 96-Well | 120 hr | RPMI1640 | 5,000 | 10 to 45 |
| 453 | | | 384-Well | | (+10% FBS) | 2,500 | |
| HuH7 | adherent | Sekisui | 96-Well | 120 hr | IMDM | 2,500 | 5 to 30 |
| | | Xenotech | | | (+10% FBS) | | |

### FGFR Cell Data:

A = 0.1-50 nM
B = >50-200 nM
C = >200-1000 nM
D = >1000 nM

| | KG-1 | Kato III | RT112/84 | HuH7 | MDA- |
|---|---|---|---|---|---|
| Example | IC50 nM | IC50 nM | IC50 nM | IC50 nM | MB-453 |
| 1 | | | | A | |
| 4 | C | C | B | A | |
| 6 | D | B | B | A | A |
| 7 | C | B | A | A | |
| 8 | B | B | A | A | A |
| 9 | A | B | A | A | A |
| 10 | B | B | A | A | |
| 11 | | | | A | |
| 12 | C | C | B | A | |
| 13 | | | | C | |
| 14 | B | B | A | A | A |
| 15 | B | B | A | A | |
| 16 | B | B | A | A | A |
| 17 | C | C | B | A | |
| 18 | | | | A | |
| 19 | C | D | C | A | |
| 20 | C | B | B | A | |
| 21 | C | C | B | B | |
| 22 | C | C | B | A | |
| 23 | C | C | B | A | |
| 24 | B | B | A | A | |
| 25 | B | B | A | A | |
| 26 | A | B | A | A | |
| 27 | C | C | B | B | |
| 28 | B | B | A | A | |
| 29 | | | | B | |
| 30 | | | | A | |
| 31 | | | | B | |
| 32 | | | | B | |
| 33 | C | B | B | A | |
| 34 | C | C | B | A | |
| 35 | | | | A | |
| 36 | | | | C | |
| 37 | B | B | A | A | |
| 38 | | | | B | |
| 39 | | | | B | |
| 41 | B | B | A | A | |
| 42 | B | B | A | A | |
| 43 | B | B | A | A | |
| 44 | B | B | A | A | |
| 45 | | | | B | |
| 46 | | | | D | |
| 47 | C | B | B | A | |
| 48 | C | C | B | A | |
| 49 | A | A | A | A | |
| 50 | A | A | A | A | |
| 51 | A | A | A | A | |
| 52 | C | B | B | A | |
| 53 | C | B | B | A | A |
| 54 | C | C | B | A | |
| 55 | B | B | A | A | A |
| 56 | C | B | A | A | |
| 57 | C | B | A | A | |
| 59 | C | C | A | | |
| 60 | D | D | B | | |
| 61 | B | B | A | A | |
| 62 | | | | A | |
| 63 | B | A | A | | |
| 64 | A | A | A | | |
| 65 | C | D | A | | |
| 66 | A | A | A | | |
| 67 | C | A | A | | |
| 70 | B | A | A | | |
| 71 | C | B | A | | |
| 72 | C | B | A | | |
| 73 | A | A | A | | |
| 74 | B | A | A | | |
| 76 | B | B | A | | C |
| 77 | C | B | A | | C |
| 78 | C | B | A | | C |
| 80 | B | A | A | A | A |
| 81 | B | B | A | A | A |
| 82 | A | A | A | A | |
| 83 | B | A | A | | A |
| 87 | C | C | B | A | |
| 88 | C | B | B | A | |
| 89 | A | A | A | | |
| 90 | A | A | A | | |
| 91 | A | A | A | | |
| 93 | B | A | A | | |
| 95 | A | A | A | | |
| 96 | A | A | A | | |
| 97 | B | A | A | | |
| 98 | C | C | B | B | |
| 99 | A | A | A | A | |
| 100 | B | B | A | A | |
| 101 | B | A | A | A | |
| 102 | C | C | B | A | |
| 103 | A | A | A | | |
| 105 | B | B | A | A | |
| 106 | B | B | A | | B |
| 108 | A | A | A | | |
| 109 | B | B | A | | |
| 110 | A | A | A | | |
| 111 | B | A | A | | |
| 112 | A | A | A | | |
| 113 | B | A | A | | |
| 114 | A | A | A | | |
| 115 | A | A | A | | |
| 116 | A | A | A | | |
| 117 | B | B | A | | |
| 118 | A | A | A | | |
| 119 | A | A | A | | |
| 12 | B | A | A | | |
| 122 | C | C | B | B | |
| 123 | C | C | B | A | |
| 124 | | | | B | |
| 126 | A | A | A | | |
| 127 | B | B | A | | |
| 128 | A | A | A | | |
| 129 | C | C | B | B | |
| 132 | B | B | A | | |
| 133 | B | C | B | C | |
| 134 | C | C | B | B | |
| 135 | C | C | B | B | |
| 136 | | | | B | |
| 137 | | | | B | |
| 138 | B | A | A | | B |
| 139 | B | B | A | A | |
| 140 | B | B | A | A | B |
| 141 | B | B | A | A | B |
| 142 | B | B | A | A | B |
| 143 | B | B | A | A | |
| 144 | B | B | A | A | |
| 145 | B | B | A | A | |
| 146 | B | B | A | A | |
| 147 | B | B | A | A | |
| 148 | B | B | A | A | B |
| 149 | A | A | A | | |
| 150 | B | A | A | A | B |
| 151 | B | B | A | A | B |
| 152 | C | B | A | A | |
| 153 | B | A | A | A | |
| 154 | B | B | A | | |
| 155 | B | A | A | | B |
| 156 | B | A | A | | B |
| 158 | B | A | A | | |
| 159 | A | A | A | | |
| 160 | A | A | A | | |
| 161 | A | A | A | | |
| 166 | B | A | A | | A |
| 167 | B | A | A | | A |
| 170 | D | D | D | | |
| 171 | A | A | A | | |
| 181 | B | A | A | | B |
| 182 | C | A | A | | A |
| 198 | C | A | A | A | A |
| 199 | B | B | A | A | A |
| 200 | D | D | D | | D |
| 203 | B | A | A | A | B |
| 204 | B | A | A | A | B |
| 205 | D | C | D | | D |
| 206 | B | A | A | | B |
| 207 | B | B | A | | B |
| 209 | B | B | B | | |
| 210 | C | B | A | | |
| 211 | B | A | A | | |
| 212 | B | A | A | | |
| 213 | C | B | A | | |
| 214 | B | A | A | | |
| 215 | B | B | A | | |
| 216 | B | B | A | | |
| 217 | B | A | A | | |
| 218 | A | A | A | | |
| 219 | A | A | A | | B |
| 220 | B | A | A | | B |
| 222 | A | A | A | | |
| 223 | B | A | A | | |
| 224 | B | A | A | | B |
| 225 | B | A | A | | B |
| 227 | B | A | A | | B |
| 228 | B | A | A | | B |
| 229 | B | A | A | | B |
| 232 | B | A | A | | |
| 237 | A | A | A | | B |
| 241 | B | A | A | | |
| 242 | B | B | A | | C |
| 243 | C | C | A | | B |
| 244 | B | B | A | | C |
| 245 | D | D | B | | D |
| 246 | C | B | A | | C |
| 247 | B | B | A | | B |
| 248 | C | B | A | | |
| 249 | B | A | A | | |
| 250 | C | B | A | | C |
| 251 | B | A | A | | B |
| 255 | B | A | A | | B |
| 256 | B | B | A | | B |
| 258 | C | B | A | | C |
| 259 | B | A | A | | A |
| 260 | B | B | A | | A |
| 261 | B | A | A | | B |
| 262 | B | B | A | | C |
| 264 | B | B | A | | B |
| 266 | C | B | A | | D |
| 267 | D | B | A | | C |
| 269 | B | A | A | | C |
| 270 | B | A | A | | A |
| 271 | C | B | A | | A |
| 272 | B | B | A | | C |
| 273 | A | A | A | | A |
| 274 | B | A | A | | C |
| 276 | B | A | A | | C |
| 277 | B | A | A | | A |
| 281 | B | A | A | | C |
| 282 | B | B | A | | B |
| 283 | A | A | A | | A |
| 284 | B | A | A | | A |
| 285 | A | A | A | | C |
| 289 | C | B | A | | C |
| 290 | B | B | A | | B |
| 294 | D | B | A | | |
| 295 | | B | A | | |
| 297 | A | A | A | | |
| 298 | B | A | A | | |
| 299 | B | B | A | | |
| 300 | D | B | A | | |
| 301 | B | A | A | | |
| 303 | B | A | A | | |
| 304 | B | A | A | | |
| 306 | C | B | A | | |
| 308 | C | B | A | | |
| 309 | B | | A | | |
| 310 | B | | A | | |
| 314 | C | B | A | | |
| 316 | B | B | A | | |
| 321 | C | C | B | A | |
| 325 | C | B | B | A | |
| 328 | D | C | B | B | |
| 329 | D | C | B | A | |
| 330 | | | | C | |
| 331 | | | | C | |
| 332 | | | | C | |
| 333 | | | | C | |
| 334 | C | B | A | A | |
| 335 | | | | C | |
| 336 | | | | A | |
| 337 | B | B | A | A | |
| 338 | B | B | A | A | |
| 339 | A | A | A | A | |
| 340 | B | B | A | A | |
| 341 | A | B | A | | |
| 342 | | | | B | |
| 343 | D | C | B | C | |
| 344 | C | B | A | A | |
| 345 | B | A | A | A | |
| 346 | B | A | A | A | |
| 347 | | | | A | |
| 348 | | | | A | |
| 349 | | | | A | |
| 350 | | | | A | |
| 351 | A | A | A | A | |
| 352 | A | A | A | A | |
| 353 | B | B | A | A | |
| 354 | B | A | A | A | |
| 355 | | | | B | |
| 356 | B | C | A | A | |
| 357 | B | B | B | A | |
| 358 | | | | A | |
| 359 | B | B | B | A | |
| 360 | B | B | A | A | |
| 361 | C | B | B | B | |
| 362 | B | B | A | A | |
| 363 | C | B | A | A | |
| 364 | D | B | B | A | |
| 365 | A | B | A | A | |
| 366 | | | | A | |
| 367 | B | A | A | A | A |
| 368 | | | | A | |
| 369 | B | A | A | A | A |
| 370 | | | | A | |
| 376 | C | B | A | | |
| 377 | C | B | A | | |
| 378 | D | B | A | | |
| 384 | B | B | A | | |
| 386 | B | A | A | | |
| 387 | C | B | A | | |
| 388 | C | B | A | | |
| 390 | B | A | A | | |
| 391 | B | A | A | | |
| 392 | B | A | A | | |
| 409 | | | | A | |
| 410 | | | | A | |
| 411 | | | | A | |

1. A compound of formula (I): 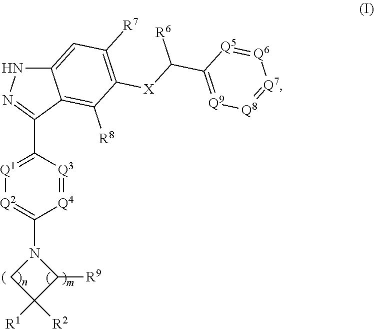
or a pharmaceutically acceptable salt thereof, wherein
X═O, S, or NR;
R is H or C₁-C₃alkyl;
n=1 or 2;
m=1 or 2;
R¹ is H, optionally substituted C₁-C₆alkyl, optionally substituted C₂-C₆alkenyl, C₃-C₆cycloalkyl, —C(O)NR³R⁴, —C(O)OR³, optionally substituted heterocycloalkyl, or optionally substituted heteroaryl;
R⁹ is H or C₁-C₃alkyl;
R² is H, optionally substituted C₁-C₆alkyl, —NR³R⁴, —OR^{4a}, —P(O)R^{4b}R^{4c}, —SO₂R³, or —C(O)NR³R⁴;
R³ is H or C₁-C₆alkyl;
R⁴ is H, optionally substituted C₁-C₆alkyl, optionally substituted C₂-C₆alkenyl, C₃-C₅cycloalkyl, 3- to 6-membered heterocycloalkyl, C(O)(CH₂)₂₋₃OH, or C(O)(CH₂)₀₋₃NR^{4d}R^{4e};
or R³ and R⁴, together with the N atom to which they are both attached, form a 3- to 6-membered heterocycloalkyl optionally substituted with one or more substituents that are each C₁-C₆alkyl, C₁-C₆alkoxyl, F, or OH;
or R³ and R⁴, together with the N atom to which they are both attached, form a 6- to 8-membered bridged heterocycloalkyl ring system;
R^{4a} is H, optionally substituted C₁-C₆alkyl or C₃-C₅cycloalkyl;
R^{4b} and R^{4c} are each independently C₁-C₆alkyl or —OC₁-C₆alkyl; or R^{4b} and R^{4c} together with the phosphorus atom to which they are both attached, form a 4-to 6-membered heterocycloalkyl ring;
R^{4d} and R^{4c} are each independently H or C₁-C₆alkyl, or R^{4d} and R^{4e}, together with the nitrogen atom to which they are both attached, form a 4- to 6-membered heterocycloalkyl ring;
or R¹ and R², together with the carbon atom to which they are both attached, form a 3-5 membered cycloalkyl ring;
one or two of Q¹, Q², Q³, Q⁴ is N and the others are each independently CR^{5a};
R^{5a} is H, halogen, —CN, —S(O)₂C₁-C₆alkyl, OCF₃, OC₁-C₃alkyl, or C₁-C₃alkyl;
Q⁵, Q⁶, Q⁷, Q⁸, and Q⁹ are each independently N or CR⁵, wherein one or two of the Q⁵, Q⁶, Q⁷, Q⁸, and Q⁹ is N and the remainder are CR⁵;
R⁵ is H, halogen, C₁-C₃alkyl, C₁-C₃alkoxyl, or cycloalkyl;
R⁶ is C₁-C₆alkyl;
R⁷ is H, halogen, —C₁-C₆alkyl, —C₁-C₆ alkoxyl, or -cycloalkyl; and
R⁸ is H, halogen, —C₁-C₆alkyl, —C₁-C₆ alkoxyl, or -cycloalkyl.

2. The compound of claim 1, wherein X is O.

3. The compound of claim 1, wherein R⁶ is CH₃.

4. The compound of claim 1, wherein R⁸ is H or F.

5. The compound of claim 1, a wherein the compound of formula (I) is a compound of formula (IA): 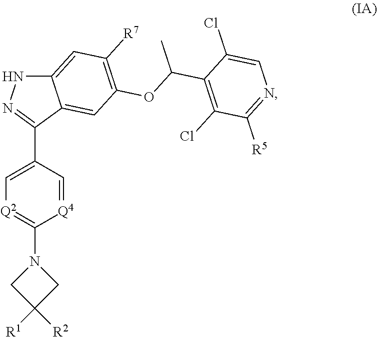
or a pharmaceutically acceptable salt thereof, wherein
Q² and Q⁴ are each N, or one of Q² or Q⁴ is N and the other is CR^{5a};
R^{5a} is H, F, —SO₂CH₃, or —CN;
R⁵ is H or CH₃; and
R⁷ is H, F, or OCH₃.

6. The compound of claim 5, wherein the compound of formula (IA) is a compound of formula (IA-1-1), (IA-2), (IA-3-1), (IA-4-1), or (IA-6): 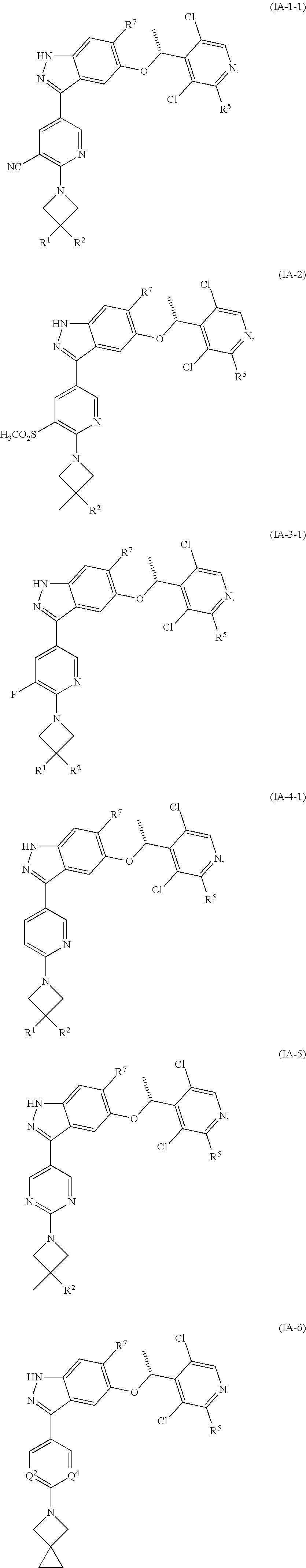
or a pharmaceutically acceptable salt thereof.

7.-11. (canceled)

12. The compound of claim 1, wherein the compound of formula (I) is a compound of formula (IB): 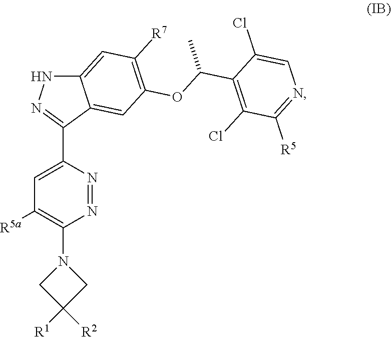
or a pharmaceutically acceptable salt thereof, wherein
R^{5a} is H, F, or —CN.

13. The compound of claim 5, wherein R⁵ is H.

14. The compound of claim 5, wherein R⁵ is CH₃.

15. The compound of claim 1, wherein R⁷ is H, F, or OCH₃.

16.-17. (canceled)

18. The compound of claim 1, wherein
Q¹ and Q³ are each CR^{5a} wherein R^{5a} is H; Q² and Q⁴ are each N; or Q³ and Q⁴ are each N.

19.-20. (canceled)

21. The compound of claim 1, wherein Q² is N and Q⁴ is CR^{5a} wherein R^{5a} is H or F.

22. The compound of claim 21, wherein the R^{5a} is H.

23. The compound of claim 21, wherein the R^{5a} is F.

24. The compound of claim 1, wherein Q² is N and Q⁴ is CR^{5a} wherein R^{5a} is CN, S(O)₂C₁-C₆alkyl, OC₁-C₃alkyl, or C₁-C₃alkyl.

25. The compound of claim 24, wherein the R^{5a} is CN or SO₂CH₃.

26.-27. (canceled)

28. The compound of claim 1, wherein Q⁴ is CR^{5a} wherein R^{5a} is OCF₃.

29. The compound of claim 1, wherein Q⁵ and Q⁹ are each CR⁵ wherein each R⁵ is Cl; Q⁷ is N; Q⁶ is CR⁵ wherein R⁵ is H; and Q⁸ is CR⁵ wherein R⁵ is H or CH₃.

30. The compound of claim 29, Q⁸ is CR⁵ wherein R⁵ is H.

31. The compound of claim 1, wherein n is 1 and m is 1; n is 2 and m is 1; or n is 2 and m is 2.

32.-33. (canceled)

34. The compound of claim 1, wherein R¹ is H.

35. The compound of claim 1, wherein R¹ is optionally substituted C₁-C₆alkyl, such as methyl, ethyl, n-propyl, isopropyl, n-butyl, isosbutyl, sec-butyl, —CH₂CH₂OH, —CH₂OH, —CH₂CH₂OCH₃, —CH₂CH₂F, —CH₂CHF₂, —CH₂CF₃, —CH₂CN, —CH₂CH₂CN, —CH₂CH₂CH₂OH, —CH₂OCH₃, —CH₂OCH(CH₃)₂, —CH₂OCH₂CH₃,

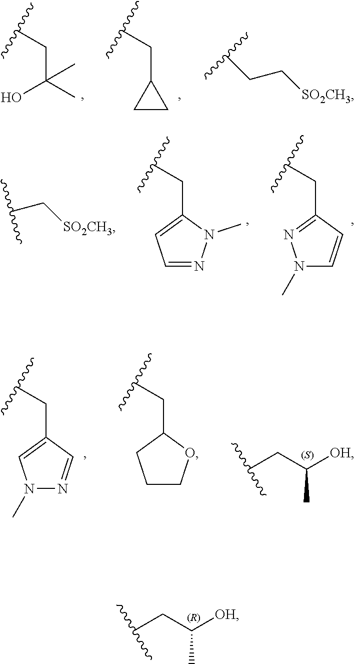

36. The compound of claim 1, wherein R¹ is optionally substituted C₂-C₆alkenyl such as

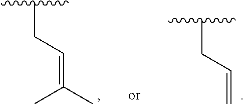

37. The compound of claim 1, wherein R¹ is C₃-C₆ cycloalkyl, such as cyclobutyl.

38. The compound of claim 1, wherein R¹ is an optionally substituted heterocycloalkyl, such as

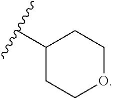 [SMILES: *C1CCOCC1]

39. The compound of claim 1, wherein R¹ is optionally substituted heteroaryl, such as

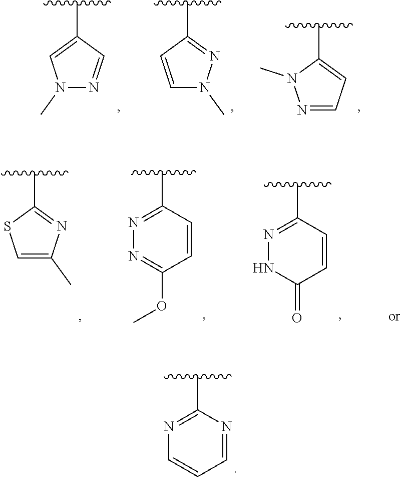

40. The compound of claim 1, wherein R1 is —C(O)OR₃, such as

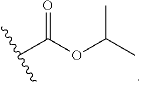 [SMILES: *C(=O)OC(C)C]

41. The compound of claim 1, wherein R¹ is —C(O)NR³R⁴, such as

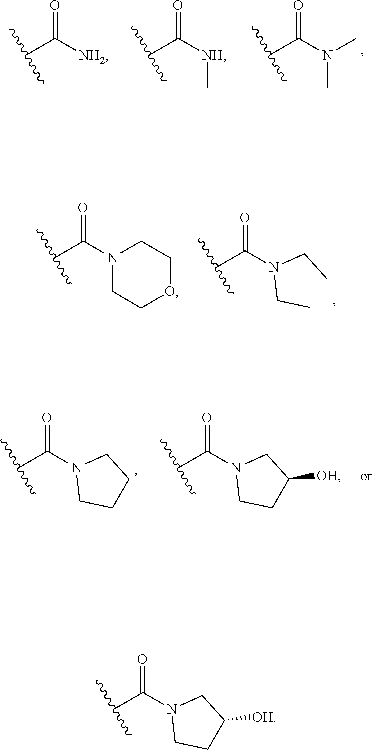

42. The compound of claim 1, wherein R² is H.

43. The compound of claim 1, wherein R² is optionally substituted C₁-C₆alkyl, such as:
C₁-C₆alkyl substituted with —NHSO₂(C₁-C₆alkyl), —N(C₁-C₆alkyl)SO₂(C₁-C₆alkyl), 5- to 6-membered heterocycloakyl, —NH(C₁-C₆alkyl), —N(C₁-C₆alkyl)₂, or —P(O)(C₁-C₆alkyl)₂;
C₁-C₆alkyl substituted with —SO₂(C₁-C₆alkyl);
C₁-C₆alkyl substituted with —NHSO₂(C₁-C₆alkyl), NHSO₂(CH₃), NHSO₂(CH₂CH₃), NHSO₂(CH₂CH₂CH₃), or NHSO₂(CH₂CH₂CH₂CH₃);
C₁-C₆alkyl substituted with —N(C₁-C₆alkyl)SO₂(C₁-C₆alkyl), —N(CH₃)SO₂(CH₃), —N(CH₃)SO₂(CH₂CH₃), —N(CH₃)SO₂(CH₂ CH₂CH₃), —N(CH₂CH₃)SO₂(CH₃), or —N(CH₂CH₃)SO₂(CH₂CH₃);
C₁-C₆alkyl substituted with 5- to 6-membered heterocycloakyl, pyrrolyl, furanyl, piperidinyl, piperazinyl, or morpholinyl;
C₁-C₆alkyl substituted with —NH(C₁-C₆alkyl), NH(CH₃), or NH(CH₂CH₃);
C₁-C₆alkyl substituted with N(C₁-C₆alkyl)₂, —N(CH₃)₂, —N(CH₂CH₃)₂, or —N(CH₃)(CH₂CH₃);
C₁-C₆alkyl substituted with NH₂, —CH₂NH₂;
C₁-C₆alkyl substituted with —P(O)R^{4b}R^{4c}, wherein R^{4b} and R^{4c} are independently C₁-C₆alkyl or —OC₁-C₆alkyl; or R^{4b} and R^{4c} together with the phosphorus atom to which they are both attached, form a 4- to 6-membered heterocycloalkyl ring;
C₁-C₆alkyl substituted with —P(O)(CH₃)₂, —P(O)(CH₂CH₃)₂, —P(O)(CH₃)(CH₂CH₃);
C₁-C₆alkyl substituted with P(O)(OCH₃)₂, —P(O)(OCH₂CH₃)₂, —P(O)(OCH₃)(OCH₂CH₃); and
C₁-C₆alkyl substituted with

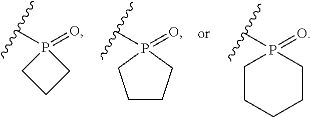

44. The compound of claim 43, wherein R² is CH₃; 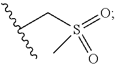 [SMILES: *CS(C)(=O)=O]
—CH₂—NHSO₂(CH₃);
—CH₂—N(CH₃)SO₂(CH₃); 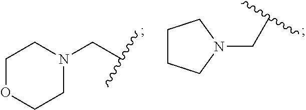
—CH₂—NH(CH₃); —CH₂—NH(CH(CH₃)₂); —CH₂—NH(CH₂CH₂OH); —CH₂—N(CH₃)₂; —CH₂—P(O)(CH₃)₂; or —CH₂—P(O)(OCH₃)₂.

45. The compound of claim 1, wherein R² is —OR^{4a} wherein R^{4a} is H, optionally substituted C₁-C₆alkyl, or C₃-C₅cycloalkyl.

46. The compound of claim 45, wherein R^{4a} is H.

47. The compound of claim 45, wherein R^{4a} is optionally substituted C₁-C₆alkyl, —CH(CH₃), —CH₂CH₂OH, or —CH₂C(CH₃)₂OH.

48. The compound of claim 45, wherein R^{4a} is C₃-C₅cycloalkyl, cyclopropyl, cyclobutyl, or cyclopentyl.

49. The compound of claim 45, wherein R² is —OH or

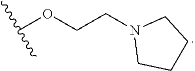 [SMILES: *OCCN1CCCC1]

50. The compound of claim 1, wherein R² is —P(O)R^{4b}R^{4c}, wherein R^{4b} and R^{4c} are independently C₁-C₆alkyl; or R^{4b} and R^{4c} together with the phosphorus atom to which they are both attached, form a 4- to 6-membered heterocycloalkyl ring.

51. The compound of claim 50, wherein R^{4b} is C₁-C₆alkyl, or CH₃.

52. The compound of claim 50, wherein R^{4c} is C₁-C₆alkyl, or CH₃.

53. The compound of claim 50, wherein R² is —P(O)(CH₃)₂.

54. The compound of claim 50, wherein R^{4b} and R^{4c} together with the phosphorus atom to which they are both attached, form a 4- to 6-membered heterocycloalkyl ring, a 4-membered heterocycloalkyl, a 5-membered heterocycloalkyl, or a 6-membered heterocycloalkyl.

55. The compound of claim 50, wherein R² is: —P(O)(CH₃)₂,

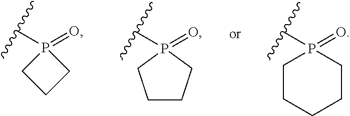 [SMILES: *P1(=O)CCC1 ; *P1(=O)CCCC1 ; *P1(=O)CCCCC1]

56. The compound of claim 1, wherein R² is —NR³R⁴.

57. The compound of wherein R² is —SO₂R³, or —SO₂CH₃.

58. The compound of claim 1, wherein R² is —C(O)NR³R⁴, —C(O)NH₂, —C(O)NHCH₃, —C(O)N(CH₃)₂, —C(O)NHCH₂CH₃, —C(O)N(CH₂CH₃)₂, or —C(O)NHCH₂CH₂OH.

59. The compound of claim 56, wherein R³ is H.

60. The compound of claim 56, wherein R³ is C₁-C₆alkyl, or CH₃.

61. The compound of claim 56, wherein R⁴ is H.

62. The compound of claim 56, wherein R⁴ is optionally substituted C₁-C₆alkyl, —CH₃, —CH₂CH₃, —CH(CH₃)₂, —CH₂CH(CH₃)₂, —CH₂CH₂CH₃, —CH(CH₃)CH₂CH₃, 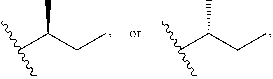
—CH₂-cyclopropyl, —CH₂CH₂SO₂CH₃, —CH₂CH₂CHF₂, —CH₂CHF₂, —CH₂CH₂OH, —CH₂CH₂OCH₃, —CH₂CH₂O-iso-Pr, —CH₂C(CH₃)₂OH, —CH₂CHCH₃OH, —CH₂CHOCH₃, 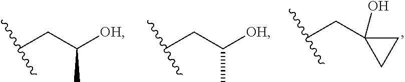
—CH₂CH₂CN, 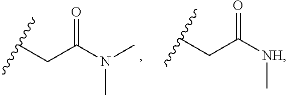
—CH₂P(O)(CH₃)₂, —CH₂CH₂P(O)(CH₂CH₃)₂, —CH₂CH₂P(O)(CH₂CH₂CH₃)₂, —CH₂CH₂P(O)(CH₃)(CH₂CH₃), —CH₂P(O), —CH₂CH₂P(O), —CH₂CH₂P(O), —CH₂CH₂P(O), or

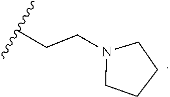 [SMILES: *CCN1CCCC1]

63. The compound of claim 56, wherein R⁴ is optionally substituted C₂-C₆alkenyl, or —CH₂CH═CH₂.

64. The compound of claim 56, wherein R⁴ is C₃-C₅cycloalkyl, or cyclobutyl.

65. The compound of claim 56, wherein R⁴ is a 3- to 6-membered heterocycloalkyl, oxetan-3-yl, thietane-3-yl-1,1-dioxide, tetrahydrofuran-3-yl

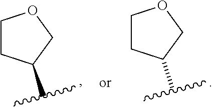 [SMILES: *[C@H]1CCOC1 ; *[C@@H]1CCOC1]

66. The compound of claim 56, wherein R⁴ is C(O)(CH₂)₀₋₃NR^{4d}R^{4e}, wherein R^{4d} and R^{4e} are each independently H or C₁-C₆alkyl, such as C(O)CH₂N(CH₃)₂, or R^{4d} and R^{4e}, together with the nitrogen atom to which they are both attached, form a 4- to 6-membered heterocycloalkyl ring, pyrrolidinyl, piperidinyl, or morpholinyl.

67. The compound of claim 56, wherein R⁴ is C(O)(CH₂)₂₋₃OH, or C(O)CH₂CH₂OH.

68. The compound of claim 56, wherein R³ and R⁴, together with the N atom to which they are both attached, form an unsubstituted 3- to 6-membered heterocycloalkyl, pyrrolidine-1-yl, azetidine-1-yl, or morpholin-4-yl.

69. The compound of claim 58, wherein R³ and R⁴, together with the N atom to which they are both attached, form a substituted 3- to 6-membered heterocycloalkyl, 3,3-dimethyl-azetidin-1-yl, 3-hydroxy-3-methylazetidin-1-yl, 1-methyl-4-azaphosphinan-4-yl 1-oxide, 3-methoxy-3-methylazetidin-1-yl,

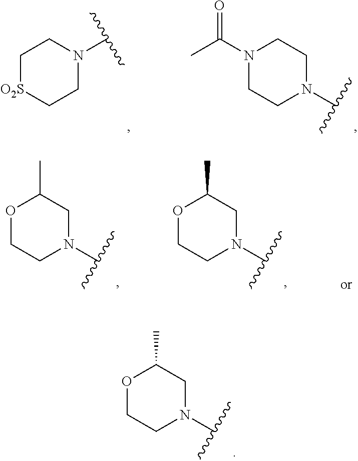

70. The compound of claim 58, wherein R³ and R⁴, together with the N atom to which they are both attached, form a 6- to 8-membered bridged heterocycloalkyl ring system,

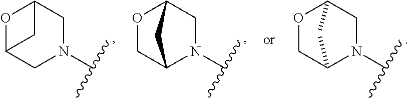

71. The compound of claim 1, wherein R¹ and R², together with the carbon atom to which they are both attached, form a 3-5 membered cycloalkyl ring, or a cyclopropyl ring.

72. A pharmaceutical composition comprising the compound of claim 1, or a pharmaceutically acceptable salt thereof, and a pharmaceutically acceptable excipient.

73. A method of treating a disease or disorder in a subject in need thereof comprising administering to the subject the compound of claim 1, or a pharmaceutically acceptable salt thereof.

74. The method of claim 73, wherein the disease or disorder is cancer.

75. The method of claim 74, wherein the cancer is urothelial carcinoma, hepatocellular carcinoma, breast carcinoma, endometrial adenocarcinoma, ovarian carcinoma, primary glioma, cholangiocarcinoma, gastric adenocarcinoma, non-small cell lung carcinoma, pancreatic exocrine carcinoma, oral cancer, prostate cancer, bladder cancer, colorectal carcinoma, renal cell carcinoma, neuroendocrine carcinoma, myeloproliferative neoplasms, head and neck (squamous), melanoma, leiomyosarcoma, and/or sarcomas.

76. The method of claim 75, wherein the cancer is bladder cancer.

77. The method of claim 75, wherein the cancer is urothelial carcinoma.

78. The method of claim 75, wherein the cancer is hepatocellular carcinoma.

79. The method of claim 74, wherein the cancer is an FGFR-mutant cancer.

80. The method of claim 73, wherein the disease or disorder is a developmental disorder.

81. The method of claim 80, wherein the developmental disorder is Achondroplasia (Ach) and related chondrodysplasia syndromes, including Hypochondroplasia (Hch), severe achondroplasia with developmental delay and Acanthosis Nigricans (SADDAN), and Thanatophoric dysplasia (TD).

82. A method of inhibiting FGFR in a cell comprising contacting the cell with the compound of claim 1.
